SEQUENCE LISTING (12 sequences)

SEQ ID NO: 1 - protein, 9 aa, Artificial Sequence
  CDRH1
FTFSSYSMN

SEQ ID NO: 2 - protein, 17 aa, Artificial Sequence
  CDRH2
YISSSSSTIYYADSVKG

SEQ ID NO: 3 - protein, 8 aa, Artificial Sequence
  CDRH3
GGHGYFDL

SEQ ID NO: 4 - protein, 12 aa, Artificial Sequence
  CDRL1
RASQSVSSSYLA

SEQ ID NO: 5 - protein, 7 aa, Artificial Sequence
  CDRL2
GASSRAT

SEQ ID NO: 6 - protein, 9 aa, Artificial Sequence
  CDRL3
QQYHHSPLT

SEQ ID NO: 7 - protein, 11 aa, Artificial Sequence
  CDRL1
SASQDIGNFLN

SEQ ID NO: 8 - protein, 7 aa, Artificial Sequence
  CDRL2
YTSSLYS

SEQ ID NO: 9 - protein, 9 aa, Artificial Sequence
  CDRL3
QQYSKLPLT

SEQ ID NO: 10 - protein, 10 aa, Artificial Sequence
  CDRH1
GYTFTGYNIH

SEQ ID NO: 11 - protein, 17 aa, Artificial Sequence
  CDRH2
AIYPGNGDTSYKQKFRG

SEQ ID NO: 12 - protein, 10 aa, Artificial Sequence
  CDRH3
GETARATFAY

(19) United States
(12) Patent Application Publication
(10) Pub. No.: US 2022/0305127 A1
(43) Pub. Date: Sep. 29, 2022

(54) PYRROLOBENZODIAZEPINE ANTIBODY CONJUGATES

(71) Applicant: Mersana Therapeutics, Inc., Cambridge, MA (US)

(72) Inventors: Joshua D. THOMAS, Natick, MA (US); Brian D. JONES, Allston, MA (US); Eugene W. KELLEHER, Wellesley, MA (US); Timothy B. LOWINGER, Carlisle, MA (US); Liping YANG, Arlington, MA (US); Mao YIN, Needham, MA (US); Aleksandr V. YURKOVETSKIY, Littleton, MA (US)

(21) Appl. No.: 16/955,346

(22) PCT Filed: Dec. 21, 2018
(86) PCT No.: PCT/US2018/067179

§ 371 Date: Jun. 18, 2020

Related U.S. Application Data
(60) Provisional application No. 62/751,941, filed on Oct. 29, 2018.
(60) Provisional application No. 62/697,640, filed on Jul. 13, 2018.
(60) Provisional application No. 62/645,512, filed on Mar. 20, 2018.
(60) Provisional application No. 62/608,778, filed on Dec. 21, 2017.

(51) Int. Cl. A61K 47/50; C07G 99/00

(52) U.S. Cl. CPC A61K 47/50; C07G 17/00

(57) ABSTRACT

The present disclosure relates generally to antibody-drug conjugates comprising pyrrolo[2, 1-c][1, 4]benzodiazepine (PBD) drug moieties. The present disclosure also relates to methods of using these conjugates, e.g., as therapeutics and/or diagnostics.

### RELATED APPLICATIONS

This application claims priority to, and the benefit of, U.S. Provisional Application Nos. 62/608,778, filed Dec. 21, 2017, 62/645,512, filed Mar. 20, 2018, 62/697,640, filed Jul. 13, 2018, and 62/751,941, filed Oct. 29, 2018, under 35 U.S.C. § 119(e). The content of these applications are hereby incorporated by reference in their entirety.

### SEQUENCE LISTING

The instant application contains a Sequence Listing which has been submitted in ASCII format via EFS-Web and is hereby incorporated by reference in its entirety. Said ASCII copy, created on Dec. 19, 2018, is named “MRSN-024_001WO_ST25.txt” and is 2,851 bytes in size.

### BACKGROUND

The pyrrolo[2, 1-c][1, 4]benzodiazepines (PBDs) are a family of naturally occurring, monofunctional DNA alkylating antitumor antibiotics, which includes anthramycin, DC-81, tomaymycin, and sibiromycin. These compounds bind exclusively to the exocyclic N2 of guanine in the minor groove and span 3 base pairs in a sequence specific manner (5′PuGPu). The first PBD antitumor antibiotic, anthramycin, was discovered in 1965 (Leimgruber et al., 1965 J. Am. Chem. Soc., 87, 5793-5795; and Leimgruber et al., 1965 J. Am. Chem. Soc., 87, 5791-5793). Since then, a number of naturally occurring PBDs and variety of analogues have been reported.

PBDs have the general structure:

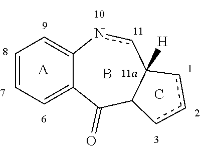

The PBDs differ in the number, type and position of substituents, in both their aromatic A rings and pyrrolo C rings, and in the degree of saturation of the C ring. In the B-ring there is either an imine (N═C), a carbinolamine (NH—CH(OH)) or a carbinolamine methyl ether (NH—CH(OMe)) at the N10-C11 position which is the electrophilic center responsible for alkylating DNA. All of the known natural products have an (S)-configuration at the chiral C11a position which provides them with a right-handed twist when viewed from the C ring towards the A ring. This gives them the appropriate three-dimensional shape for isohelicity with the minor groove of B-form DNA, leading to a snug fit at the binding site (Kohn, 1975 In Antibiotics III. Springer-Verlag, New York, pp. 3-11; and Hurley and Needham-VanDevanter, 1986 Acc. Chem. Res., 19, 230-237). Their ability to form an adduct in the minor groove enables them to interfere with DNA processing, hence their use as antitumor agents.

The first PBD to enter the clinic, SJG-136 (NSC 694501) is a potent cytotoxic agent that causes DNA inter-strand crosslinks (S. G Gregson et al., 2001, J. Med. Chem., 44: 737-748; M. C. Alley et al., 2004, Cancer Res., 64: 6700-6706; JA. Hartley et al., 2004, Cancer Res., 64: 6693-6699; C. Martin et al., 2005, Biochemistry., 44: 4135-4147; S. Arnould et al., 2006, Mol. Cancer Ther., 5: 1602-1509). Results from a Phase I clinical evaluation of SJG-136 revealed that this drug was toxic at extremely low doses (maximum tolerated dose of 45 μg/m², and several adverse effects were noted, including vascular leak syndrome, peripheral edema, liver toxicity and fatigue. DNA damage was noted at all doses in circulating lymphocytes.

Accordingly, there exists a need for more selective and efficacious drugs that can deliver critical DNA damage with minimal side effects continues.

### SUMMARY

The present disclosure provides, inter alia, an antibody-drug conjugate (ADC) of Formula (I):

PBRM-[L^{C}-D]_{d}_{₁₅} (I)

or a pharmaceutically acceptable salt or solvate thereof, wherein:

PBRM denotes a protein based recognition-molecule;

L^{C} is a linker unit connecting the PBRM to D;

D is a PBD drug moiety; and

d₁₅ is an integer from about 1 to about 20.

In some embodiments, the conjugate is of Formula (II):

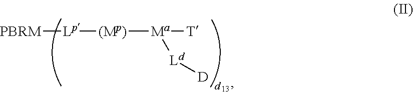

or a pharmaceutically acceptable salt or solvate thereof, wherein:

PBRM denotes a protein based recognition-molecule;

each occurrence of D is independently a PBD drug moiety;

L^{P′} is a divalent linker moiety connecting the PBRM to M^{P}; of which the corresponding monovalent moiety L^{P} contains a functional group W^{P} that is capable of forming a covalent bond with a functional group of the PBRM;

M^{P} is a Stretcher unit;

a₁ is an integer from 0 to 1;

M^{A} comprises a peptide moiety that contains at least two amino acids;

T′ is a hydrophilic group and the

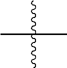

between T′ and M^{A} denotes direct or indirect attachment of T′ and M^{A};

each occurrence of L^{D} is independently a divalent linker moiety connecting D to M^{A} and comprises at least one cleavable bond such that when the bond is broken, D is released in an active form for its intended therapeutic effect; and

d₁₃ is an integer from 1 to 14.

In some embodiments, d₁₃ is an integer from 2 to 14, from 2 to 12, from 2 to 10, from 2 to 8, from 2 to 6, from 2 to 4, from 4 to 10, from 4 to 8, from 4 to 6, from 6 to 14, from 6 to 12, from 6 to 10, from 6 to 8, from 8 to 14, from 8 to 12, or from 8 to 10.

In some embodiments, d₁₃ is 3 to 5.

In some embodiments, d₁₃ is 4 or 5.

In some embodiments, L^{P}, when not connected to PBRM, comprises a terminal group W^{P}, in which each W^{P} independently is:

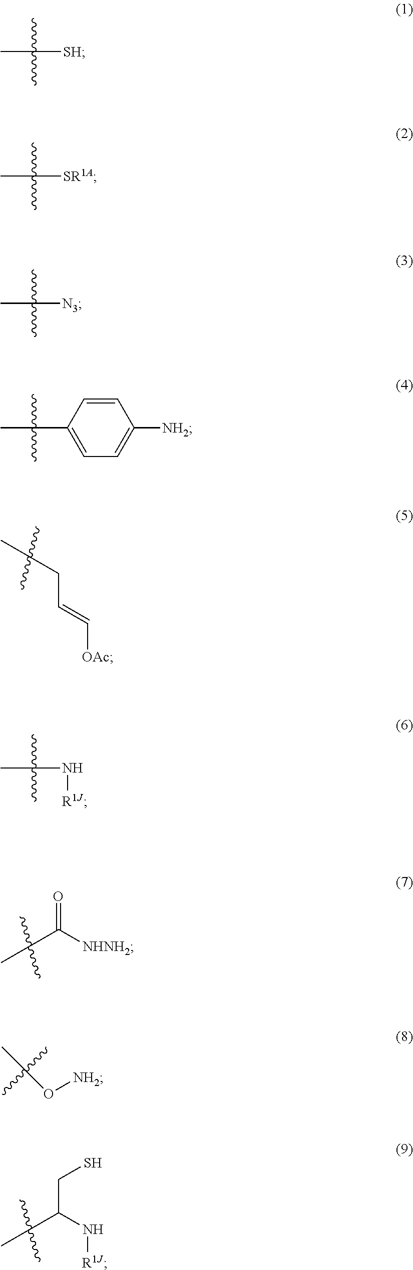

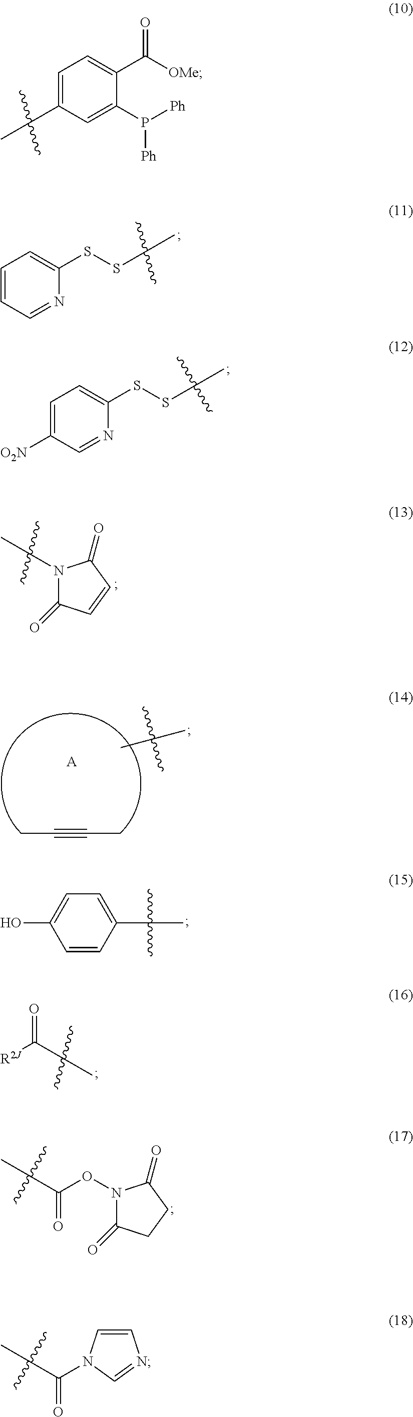

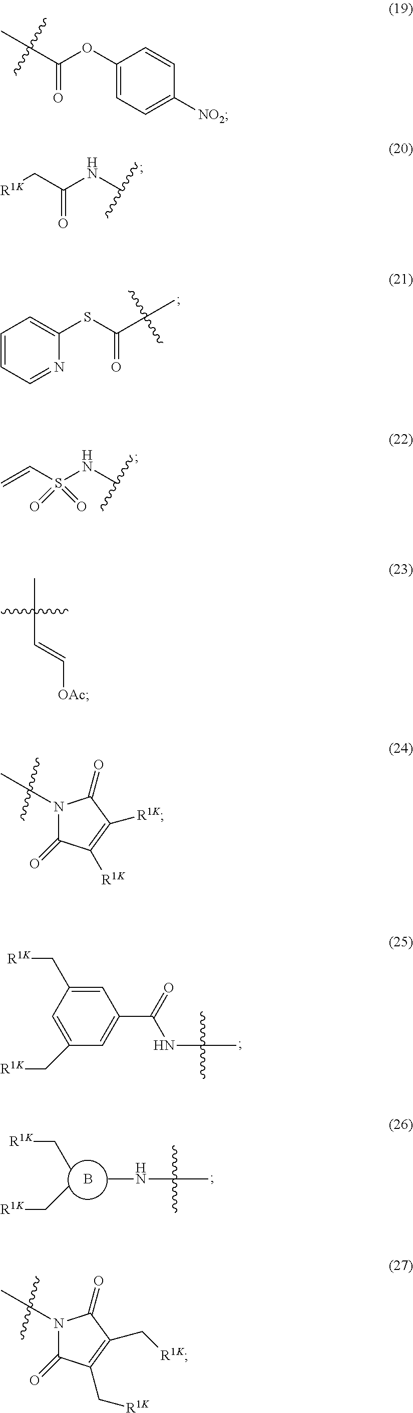

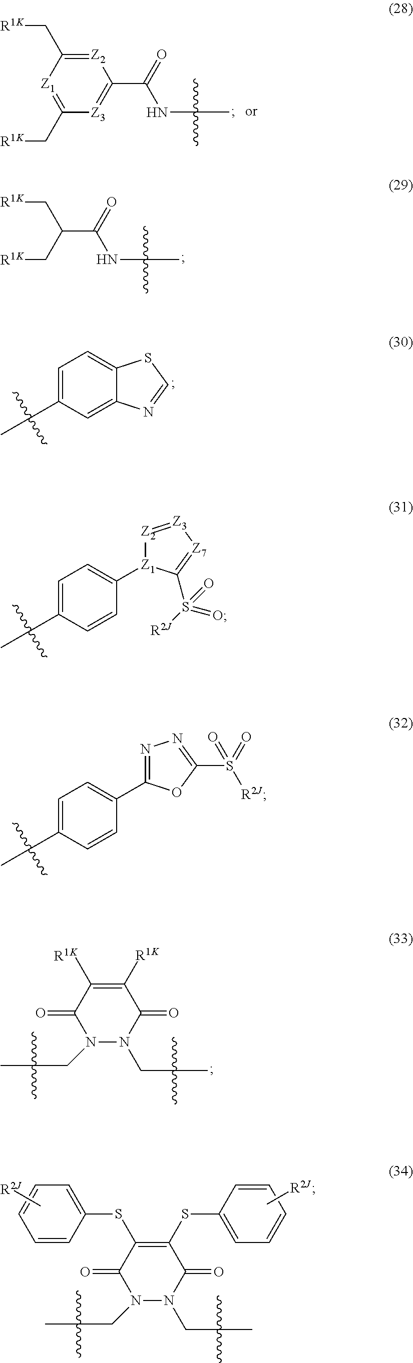

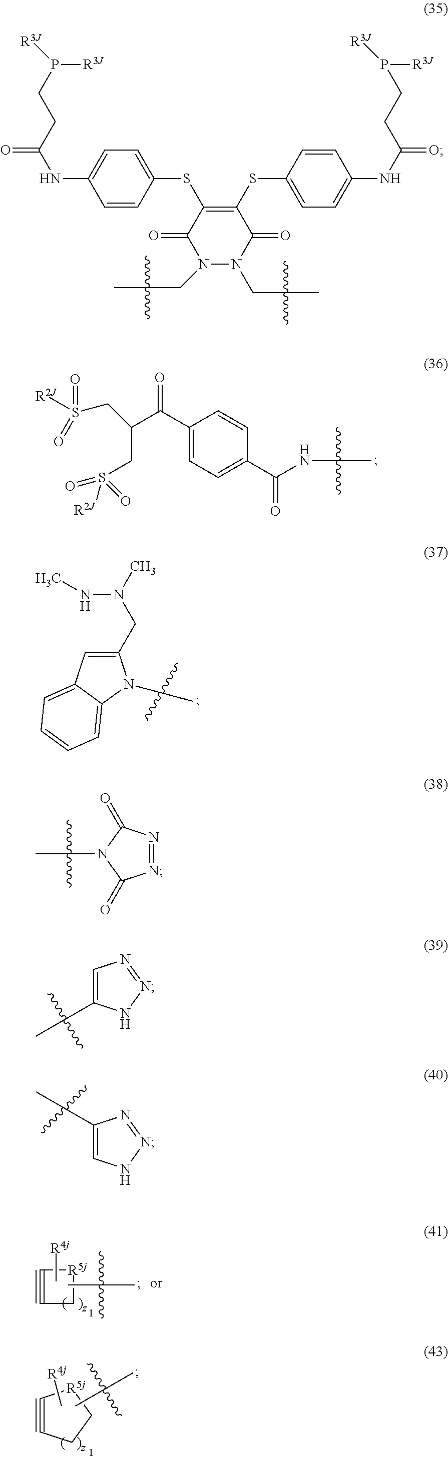

wherein

R^{1K} is a leaving group;

R^{1A} is a sulfur protecting group;

ring A is cycloalkyl or heterocycloalkyl;

ring B is cycloalkyl, heterocycloalkyl, aryl, or heteroaryl;

R^{1J} is hydrogen or an aliphatic, heteroaliphatic, carbocyclic, or heterocycloalkyl moiety;

R^{2J} is hydrogen or an aliphatic, aryl, heteroaliphatic, or carbocyclic moiety;

R^{3J} is C₁₋₆ alkyl;

Z₁, Z₂, Z₃ and Z₇ are each independently a carbon or nitrogen atom;

R^{4j} is hydrogen, halogen, OR, —NO₂, —CN, —S(O)₂R, C₁₋₂₄ alkyl (e.g., C₁₋₆ alkyl), or 6-24 membered aryl or heteroaryl, wherein the C₁₋₂₄ alkyl (e.g., C₁₋₆ alkyl), or 6-24 membered aryl or heteroaryl, is optionally substituted with one or more aryl or heteroaryl; or two R⁴ together form an annelated cycloalkyl, heterocycloalkyl, aryl, or heteroaryl;

R is hydrogen or an aliphatic, heteroaliphatic, carbocyclic, or heterocycloalkyl moiety;

R^{5j} is C(R⁴)₂, O, S or NR; and

z₁ is an integer 1, 2, 3, 4, 5, 6, 7, 8, 9 or 10.

In some embodiments, each R^{1K} is halo or RC(O)O— in which R is hydrogen or an aliphatic, heteroaliphatic, carbocyclic, or heterocycloalkyl moiety.

In some embodiments, each R^{1A} independently is

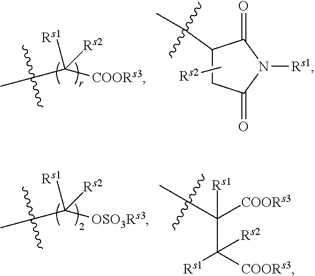

in which r is 1 or 2 and each of R^{s1}, R^{s2}, and R^{s3} is hydrogen or an aliphatic, heteroaliphatic, carbocyclic, or heterocycloalkyl moiety.

In some embodiments, L^{P}, when not connected to PBRM is

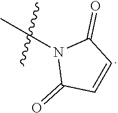 [SMILES: *N1C(=O)C=CC1=O]

In some embodiments, M^{P}, when present, is —(Z₄)—[(Z₅)—(Z₆)]_{z}—, with Z connected to L^{P′} or L^{P} and Z₆ connected to L^{M}; in which

z is 1, 2, or 3;

Z₄ is:

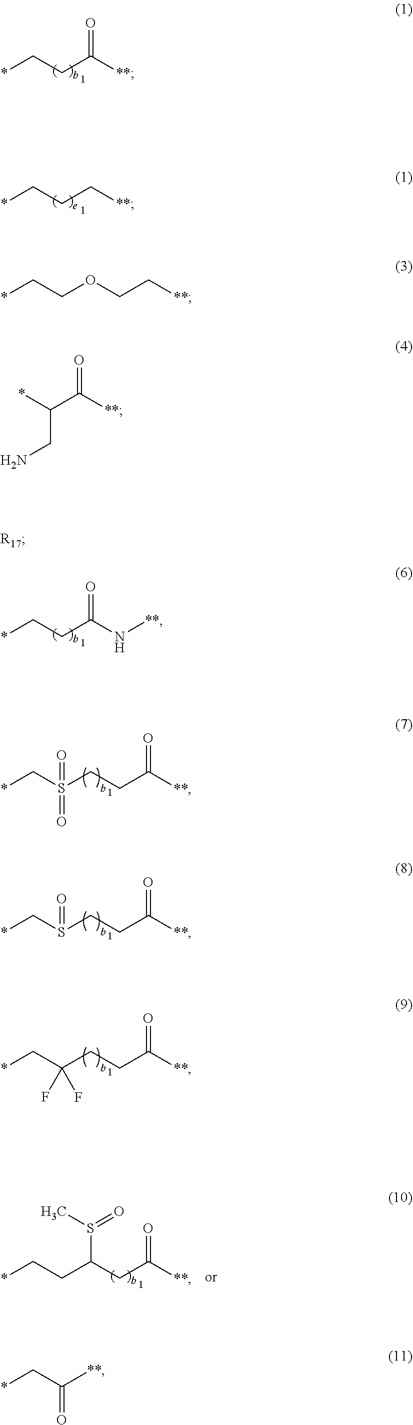

wherein * denotes attachment to L^{P′} or L^{P} and ** denotes attachment to Z₅ or Z₆ when present or to M^{A} when Z₅ and Z₆ are both absent;

b₁ is an integer from 0 to 6;

e₁ is an integer from 0 to 8,

R₁₇ is C₁₋₁₀ alkylene, C₁₋₁₀ heteroalkylene, C₃₋₈ cycloalkylene, O—(C₁₋₈ alkylene, arylene, —C₁₋₁₀ alkylene-arylene-, -arylene-C₁₋₁₀ alkylene-, —C₁₋₁₀ alkylene-(C₃₋₈ cycloalkylene)-, —(C₃₋₈ cycloalkylene —C₁₋₁₀ alkylene-, 4 to 14-membered heterocycloalkylene, —C₁₋₁₀ alkylene-(4 to 14-membered heterocycloalkylene)-, -(4 to 14-membered heterocycloalkylene)-C₁₋₁₀ alkylene-, —C₁₋₁₀ alkylene-C(═O)—, —C₁₋₁₀ heteroalkylene-C(═O)—, —C₃₋₈ cycloalkylene-C(═O)—, —O—(C₃₋₈ alkyl)-C(═O)—, -arylene-C(═O)—, —C₁₋₁₀ alkylene-arylene-C(═O)—, -arylene —C₁₋₁₀ alkylene-C(═O)—, —C₁₋₁₀ alkylene-(C₃₋₈ cycloalkylene)-C(═O)—, —(C₃₋₈ cycloalkylene)-C₁₋₁₀ alkylene-C(═O)—, -4 to 14-membered heterocycloalkylene-C(═O)—, —C₁₋₁₀ alkylene-(4 to 14-membered heterocycloalkylene)-C(═O)—, -(4 to 14-membered heterocycloalkylene)-C₁₋₁₀ alkylene-C(═O)—, —C₁₋₁₀ alkylene-NH—, —C₁₋₁₀ heteroalkylene-NH—, —C₃₋₈ cycloalkylene-NH—, —O—(C₁₋₈ alkyl)-NH—, -arylene-NH—, —C₁₋₁₀ alkylene-arylene-NH—, -arylene-C₁₋₁₀ alkylene-NH—, —C₁₋₁₀ alkylene-(C₃₋₈-cycloalkylene)-NH—, —(C₃₋₈ cycloalkylene)-C₁₋₁₀ alkylene-NH—, -4 to 14-membered heterocycloalkylene-NH—, —C₁₋₁₀ alkylene-(4 to 14-membered heterocycloalkylene)-NH—, -(4 to 14-membered heterocycloalkylene)-C₁₋₁₀ alkylene-NH—, —C₁₋₁₀ alkylene-S—, —C₁₋₁₀ heteroalkylene-S—, —C₃₋₈ cycloalkylene-S—, —O—C₁₋₈ alkyl)-S—, -arylene-S—, —C₁₋₁₀ alkylene-arylene-S—, -arylene-C₁₋₁₀ alkylene-S—, —C₁₋₁₀ alkylene-(C₃₋₈ cycloalkylene)-S—, —(C₃₋₈-cycloalkylene)-C₁₋₁₀ alkylene-S—, -4 to 14-membered heterocycloalkylene-S—, —C₁₋₁₀ alkylene-(4 to 14-membered heterocycloalkylene)-S—, or -(4 to 14-membered heterocycloalkylene)-C₁-C₁₀ alkylene-S—;
each Z₅ independently is absent, R₅₇-R₁₇ or a polyether unit;
each R₅₇ independently is a bond, NR₂₃, S or O;
each R₂₃ independently is hydrogen, C₁₋₆ alkyl, C₆₋₁₀ aryl, C₃₋₈ cycloalkyl, —COOH, or —COO—C₁₋₆ alkyl; and

each Z₆ independently is absent, —C₁₋₁₀ alkyl-R₃—, —C₁₋₁₀ alkyl-NR₅—, —C₁₋₁₀ alkyl-C(O)—, —C₁₋₁₀ alkyl-O—, —C₁₋₁₀ alkyl-S— or —(C₁₋₁₀ alkyl-R₃)_{g1}—C₁₋₁₀ alkyl-C(O)—;

each R₃ independently is —C(O)—NR₅— or —NR₅—C(O)—;
each R₅ independently is hydrogen, C₁₋₆ alkyl, C₆₋₁₀ aryl, C₃₋₈ cycloalkyl, COOH or COO—C₁₋₆ alkyl; and

g₁ is an integer from 1 to 4.

In some embodiments, Z₄ is

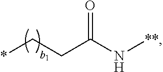

in which b₁ is 1 or 4.

In some embodiments, Z₄ is

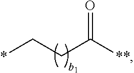

in which b₁ is 1.

In some embodiments, Z₄ is

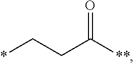

in which b₁ is 1.

In some embodiments, Z₄ is

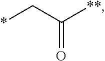

in which b₁ is 0.

In some embodiments, each Z₅ independently is a polyalkylene glycol (PAO).

In some embodiments, M^{P}, when resent, is

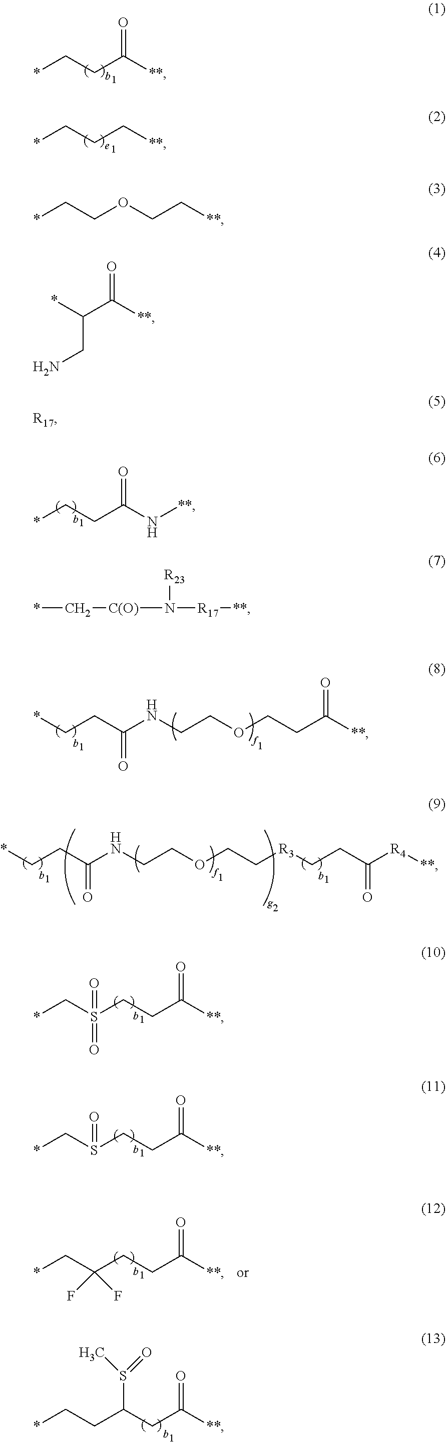

wherein * denotes attachment to L^{P′} or L^{P} and ** denotes attachment to L^{M};

R₃, R₅, R₁₇, and R₂ are as defined herein:

R₄ is a bond or —NR—(CR₂₀R₂₁)—C(O)—;

each R₂₀ and R₂₁ independently is hydrogen, C₁₋₆ alkyl, C₆₋₁₀ aryl, hydroxylated C₆₋₁₀ aryl, polyhydroxylated C₆₋₁₀ aryl, 5 to 12-membered heterocycle, C₃₋₈ cycloalkyl, hydroxylated C₃₋₈ cycloalkyl, polyhydroxylated C₃₋₈ cycloalkyl or a side chain of a natural or unnatural amino acid;

each b₁ independently is an integer from 0 to 6;

e₁ is an integer from 0 to 8,

each f₁ independently is an integer from 1 to 6; and

g₂ is an integer from 1 to 4.

In some embodiments, M^{P}, when present, is:

wherein * denotes attachment to L^{P′} or L^{P} and ** denotes attachment to L^{M}.

In some embodiments, M^{P}, when present, is:

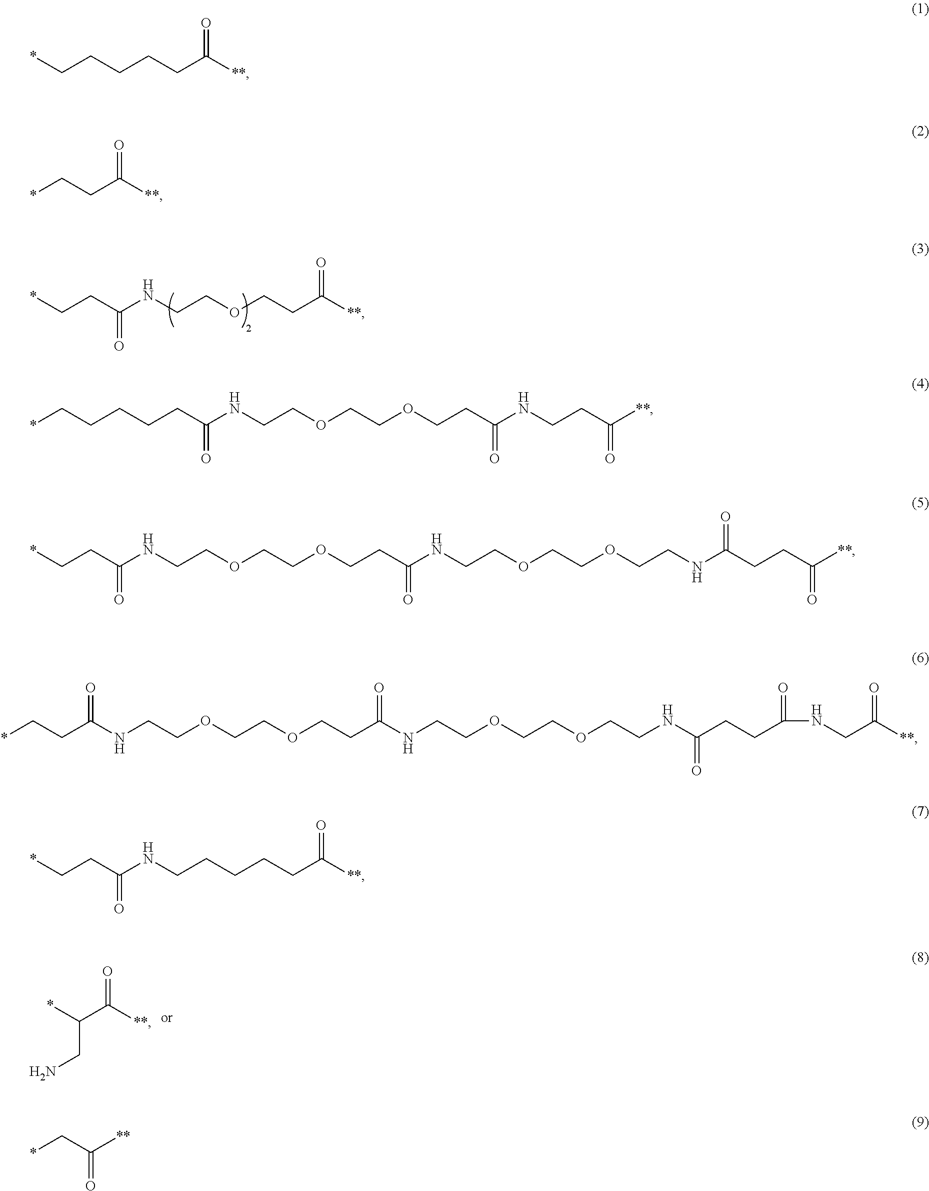

wherein * denotes attachment to L^{P′} or L^{P} and ** denotes attachment to M^{A}.

In some embodiments, M^{P}, when present, is:

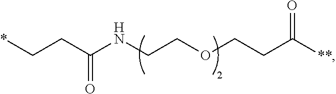

wherein * denotes attachment to L^{P′} or L^{P} and ** denotes attachment to M^{A}.

In some embodiments, M^{A} comprises a peptide moiety of at least two amino acid (AA) units.

In some embodiments, L^{D} comprises a peptide of 1 to 12 amino acids, wherein each amino acid is independently selected from alanine, β-alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, selenocysteine, ornithine, penicillamine, aminoalkanoic acid, aminoalkynoic acid, aminoalkanedioic acid, aminobenzoic acid, amino-heterocyclo-alkanoic acid, heterocyclo-carboxylic acid, citrulline, statine, diaminoalkanoic acid, and derivatives thereof.

In some embodiments, L^{D} comprises β-alanine.

In some embodiments, L^{D} comprises (β-alanine)-(alanine)-(alanine) or (β-alanine)-(valine)-(alanine).

In some embodiments, T′ comprises a polyalcohol or a derivative thereof, a polyether or a derivative thereof, or a combination thereof.

In some embodiments, T′ comprises an amino polyalcohol.

In some embodiments, T′ comprises one or more of the following fragments of the formula:

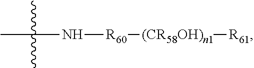

n₁ is an integer from 0 to about 6;

each R₅₈ independently hydrogen or C₁₋₈ alkyl;

R₆₀ is a bond, a C₁₋₆ alkyl linker, or —CHR₅₉— in which R₅₉ is H, alkyl, cycloalkyl, or arylalkyl;

R₆₁ is CH₂OR₆₂, COOR₆₂, —(CH₂)n2COOR₆₂,or a heterocycloalkyl substituted with one more hydroxyl;

R₆₂ is H or C₁₋₈ alkyl, and

n₂ is an integer from 1 to about 5.

In some embodiments, T′ comprises glucamine.

In some embodiments, T′ comprises:

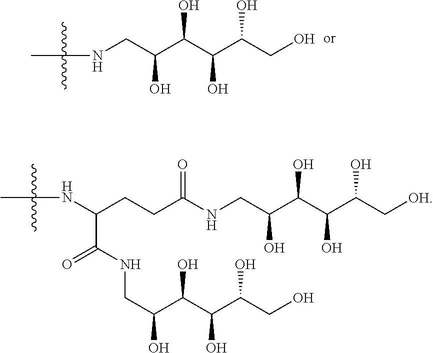 [SMILES: *NC[C@H](O)[C@@H](O)[C@H](O)[C@H](O)CO ; *NC(CCC(=O)NC[C@H](O)[C@@H](O)[C@H](O)[C@H](O)CO)C(=O)NC[C@H](O)[C@@H](O)[C@H](O)[C@H](O)CO]

In some embodiments, T′ comprises:

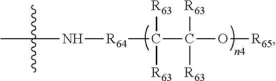

in which

n₄ is an integer from 1 to about 25;

each R₆₃ is independently hydrogen or C₁₋₈ alkyl;

R₆₄ is a bond or a C₁₋₈ alkyl linker; R₆₅ is H, C₁₋₈ alkyl, —(CH₂)ₙ₂COOR₆₂, or —(CH₂)ₙ₂COR₆₆; R₆₂ is H, or C₁₋₈ alkyl;

R₆₆ is

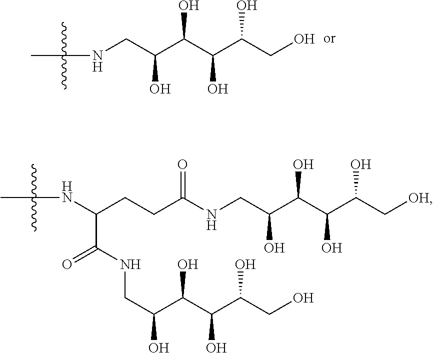 [SMILES: *NC[C@H](O)[C@@H](O)[C@H](O)[C@H](O)CO ; *NC(CCC(=O)NC[C@H](O)[C@@H](O)[C@H](O)[C@H](O)CO)C(=O)NC[C@H](O)[C@@H](O)[C@H](O)[C@H](O)CO]

and

n₂ is an integer from 1 to about 5.

In some embodiments, T′ comprises polyethylene glycol, e.g., polyethylene glycol with from about 6 to about 24 PEG subunits, preferably from about 6 to about 12 PEG subunits, or from about 8 to about 12 PEG subunits.

In some embodiments T′ comprises:

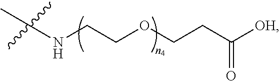

in which n₄ is an integer from about 2 to about 20, from about 4 to about 16, from about 6 to about 12, from about 8 to about 12.

In some embodiments, n₄ is 6, 7, 8, 9, 10, 11, or 12.

In some embodiments, n₄ is 8 or 12.

In some embodiments, T′ comprises:

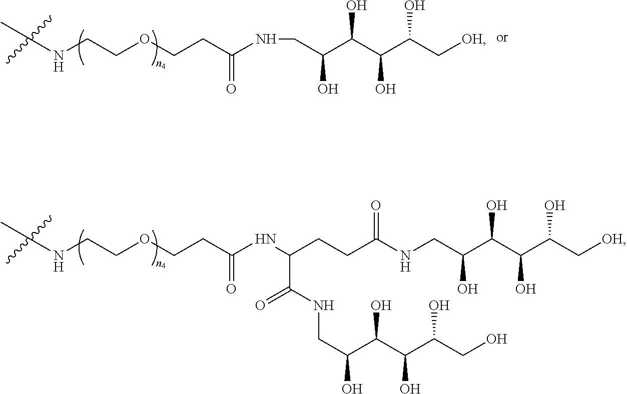

in which n₄ is an integer from about 2 to about 20, from about 4 to about 16, from about 6 to about 12, or from about 8 to about 12.

In some embodiments, n₄ is 6, 7, 8, 9, 10, 11, or 12.

In some embodiments, n₄ is 8 or 12.

In some embodiments, the conjugate is of Formula (III):

PBRM-(A¹ₐ₆-L¹ₛ₂-L²_{y1}-D)_{d13} (III)

or pharmaceutically acceptable salt or solvate thereof, wherein:

PBRM denotes a protein based recognition-molecule;

each occurrence of D is independently a PBD drug moiety;

A¹ is a stretcher unit;

a₆ is an integer 1 or 2;

L¹ is a specificity unit;

s₂ is an integer from about 0 to about 12;

L² is a spacer unit;

y1 is an integer from 0 to 2; and

d₁₃ is an integer from about 1 to about 14.

In some embodiments, the conjugate is of any one of Formulae (IIIa) to (IIIf):

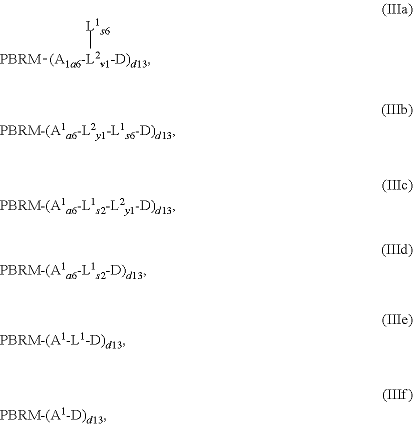

or a pharmaceutically acceptable salt or solvate thereof, wherein:

PBRM denotes a protein based recognition-molecule;

each occurrence of D is independently a PBD drug moiety;

A¹ is a stretcher unit linked to the spacer unit L²;

a₆ is an integer 1 or 2:

L¹ is a specificity unit linked to the spacer unit L²;

s₂ is an integer from about 0 to about 12;

s₆ is an integer from about 0 to about 12;

L² is a spacer unit;

y₁ is an integer 0, 1 or 2; and

d₁₃ is an integer from about 1 to about 14.

In some embodiments, the PBD drug moiety (D) is of Formula (IV):

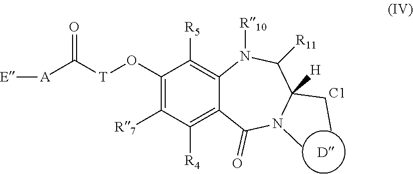

a tautomer thereof; a pharmaceutically acceptable salt or solvate thereof; or a pharmaceutically acceptable salt or solvate of the tautomer, wherein:

E″ is a direct or indirect linkage to the PBRM (e.g., antibody or antibody fragment), E, or

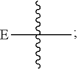

in which

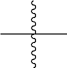

denotes direct or indirect linkage to the PBRM (e.g., antibody or antibody fragment) via a functional group of E;

D″ is D′ or

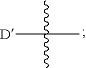

in which

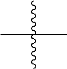

denotes direct or indirect linkage to the PBRM (e.g., antibody or antibody fragment) via a functional group of D′;

R″₇ is a direct or indirect linkage to the PBRM (e.g., antibody or antibody fragment), R₇, or

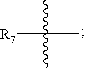

in which

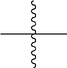

denotes direct or indirect linkage to the PBRM (e.g., antibody or antibody fragment) via a functional group of R₇;

R″₁₀ is a direct or indirect linkage to the PBRM (e.g., antibody or antibody fragment), R₁₀, or

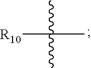

in which

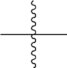

denotes direct or indirect linkage to the PBRM (e.g., antibody or antibody fragment) via a functional group of R₁₀; and

wherein the PBD drug moiety (D) is directly or indirectly linked to the PBRM (e.g., antibody or antibody fragment) via a functional group of one of E″, D″, R″₇, and R″₁₀.

In some embodiments, E″ is a direct or indirect linkage to L^{C}, E, or

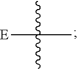

in which

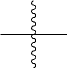

denotes direct or indirect linkage to L^{C} via a functional group of E.

In some embodiments, E″ is a direct or indirect linkage to L^{D}, E, or

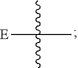

in which

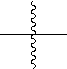

denotes direct or indirect linkage to L^{D} via a functional group of E.

In some embodiments, D″ is D′ or

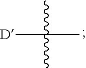

in which

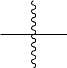

denotes direct or indirect linkage to L^{C} via a functional group of D′.

In some embodiments, D″ is D′ or

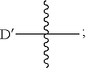

in which

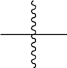

denotes direct or indirect linkage to L^{D} via a functional group of D′.

In some embodiments, R″₇ is a direct or indirect linkage to L^{C}, R₇ or

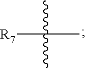

in which

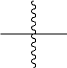

denotes direct or indirect linkage to L^{C} via a functional group of R₇.

In some embodiments, R″₇ is a direct or indirect linkage to L^{D}, R₇ or

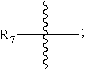

in which

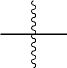

denotes direct or indirect linkage to L^{D} via a functional group of R₇.

In some embodiments, R″₁₀ is a director indirect linkage to L^{C}, R₁₀, or

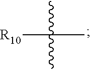

in which

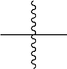

denotes direct or indirect linkage L^{C} via a functional group of R₁₀.

In some embodiments, R″₁₀ is a direct or indirect linkage to L^{D}, R₁₀, or

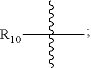

in which

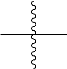

denotes direct or indirect linkage L^{C} via a functional group of R₁₀.

In some embodiments, E″ is a direct or indirect linkage to the PBRM; D″ is D′; R″₇ is R₇ and R″₁₀ is R₁₀.

In some embodiments, E″ is a director indirect linkage to L^{C}; D″ is D′; R″₇ is R₇ and R″₁₀ is R₁₀.

In some embodiments, E″ is a direct or indirect linkage to L^{D}; D″ is D′; R″₇ is R, and R″₁₀ is R₁₀.

In some embodiments, E″ is

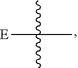

in which

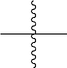

denotes direct or indirect linkage to the PBRM via a functional group of E; D″ is D′; R″₇ is R₇; and R″₁₀ is R₁₀.

In some embodiments, E″ is

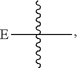

in which

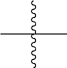

denotes direct or indirect linkage to L^{C} via a functional group of E; D″ is D′; R″₇ is R₇; and R″₁₀ is R₁₀.

In some embodiments, E″ is

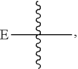

in which

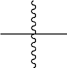

denotes direct or indirect linkage to L^{D} via a functional group of E; D″ is D′; R″₇ is R₇; and R″₁₀ is R₁₀.

In some embodiments, D″ is

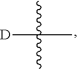 [SMILES: *[2H]]

in which

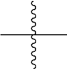

denotes director indirect linkage to the PBRM via a functional group of D; E″ is E; R″₇ is R₇; and R″₁₀ is R₁₀.

In some embodiments, D″ is

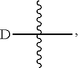 [SMILES: *[2H]]

in which

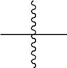

denotes direct or indirect linkage to L^{C} via a functional group of D; E″ is E; R″₇ is R₇; and R″₁₀ is R₁₀.

In some embodiments, D″ is

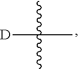 [SMILES: *[2H]]

in which

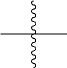

denotes direct or indirect linkage to L^{D} via a functional group of D; E″ is E; R″₇ is R₇; and R″₁₀ is R₁₀.

In some embodiments, R″₇ is a direct or indirect linkage to the PBRM; E″ is E; D″ is D′; and R″₁₀ is R₁₀.

In some embodiments, R″₇ is a direct or indirect linkage to L; E″ is E; D″ is D′; and R″₁₀ is R₁₀.

In some embodiments R″₇ is a direct or indirect linkage to L^{D}; E″ is E; D″ is D′; and R″₁₀ is R₁₀.

In some embodiments, R″₇ is

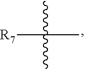

in which

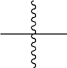

denotes direct or indirect linkage to the PBRM via a functional group of R₇; E″ is E; D″ is D′; and R″₁₀ is R₁₀.

In some embodiments, R″₇ is

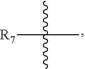

in which

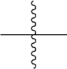

denotes direct or indirect linkage to L^{C} via a functional group of R₇; E″ is E; D″ is D′; and R″₁₀ is R₁₀.

In some embodiments, R″₇ is

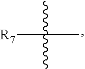

in which

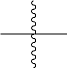

denotes direct or indirect linkage to L^{D} via a functional group of R₇; E″ is E; D″ is D′; and R″₁₀ is R₁₀.

In some embodiments, R″₁₀ is a director indirect linkage to the PBRM; E″ is E; D″ is D′; and R″₇ is R₇.

In some embodiments, R″₁₀ is a director indirect linkage to L^{C}; E″ is E, D″ is D′; and R″₇ is R₇.

In some embodiments, R″₁₀ is a direct or indirect linkage to L^{D}; E″ is E; D″ is D′; and R″₇ is R₇.

In some embodiments, R″₁₀ is

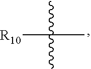

in which

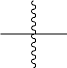

denotes direct or indirect linkage to the PBRM via a functional group of R₁₀; E″ is E; D″ is D′; and R″₇ is R₇.

In some embodiments, R″₁₀ is

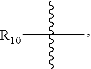

in which

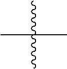

denotes direct or indirect linkage to L^{C} via a functional group of R₁₀; E″ is E; D″ is D′; and R″₇ is R₇.

In some embodiments, R″₁₀ is

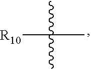

in which

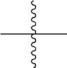

denotes direct or indirect linkage to L^{D} via a functional group of R₁₀, E″ is E; D″ is D′; and R″₇ is R₇.

In some embodiments, D′ is D1, D2, D3, or D4:

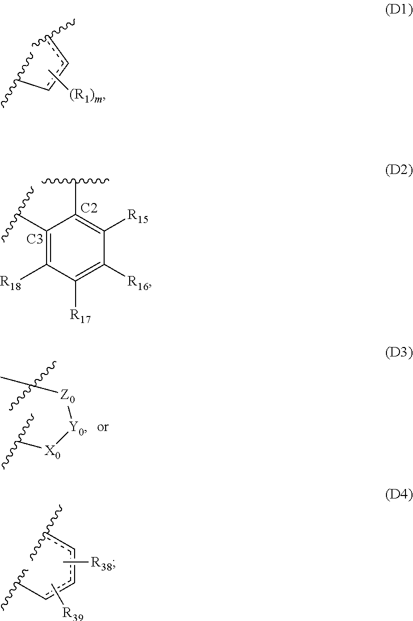

wherein the dotted line between C2 and C3 or between C2 and C1 in D1 or the dotted line in D4 indicates the presence of a single or double bond; and

m is 0, 1 or 2;

when D′ is D1, the dotted line between C2 and C3 is a double bond, and m is 1, then R₁ is:

(i) C₆₋₁₀ aryl group, optionally substituted by one or more substituents selected from —OH, halo, —NO₂, —CN, —N₃, —OR₂, —COOH, —COOR₂, —COR₂, —OCONR₁₃R₁₄. C₁₋₁₀ alkyl, C₃₋₁₀ cycloalkyl, C₂₋₁₀ alkenyl, C₂₋₁₀ alkynyl, a polyethylene glycol unit —(OCH₂CH₂)ᵣ—ORₐ, 3- to 14-membered heterocycloalkyl, 5- to 12-membered heteroaryl, bis-oxy-C₁₋₃ alkylene, —NR₁₃R₁₄, —S(═O)₂R₁₂, —S(═O)₂NR₁₃R₁₄, —SR₁₂, —SOₓM, —OSOₓM, —NR₉COR₁₉, —NH(C═NH)NH₂;

(ii) C₁₋₅ alkyl;

(iii) C₃₋₆ cycloalkyl;

(iv)

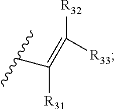

(vi)

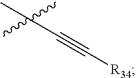

(vii)

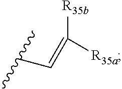

(viii)

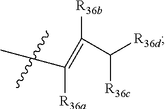

or

(viii) halo;

when D′ is D1, the dotted line between C₂ and C₃ is a single bond, and m is 1, then R₁ is:

(i) —OH, ═O, ═CH₂, —CN, —R₂, —OR₂, halo, ═CH—R₆, ═C(R₆)₂, —O—SO₂R₂, —CO₂R₂, —COR₂, —CHO, or —COOH; or (ii)

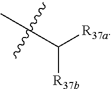

when D′ is D1 and m is 2, then each R₁ independently is halo and either both R₁ are attached to the same carbon atom or one is attached to C₂ and the other is attached to C₃;

T is C₁₋₁₀ alkylene linker;

A is

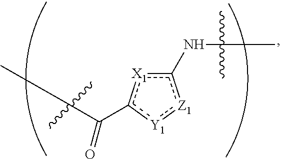

wherein the —NH group of A is connected to the —C(O)-T- moiety of Formula (IV) and the C═O moiety of A is connected to E; and each

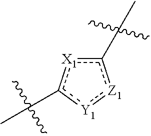

independently is

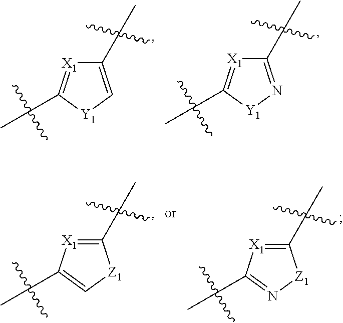

E is E1, E2, E3, E4, E5, or E6:

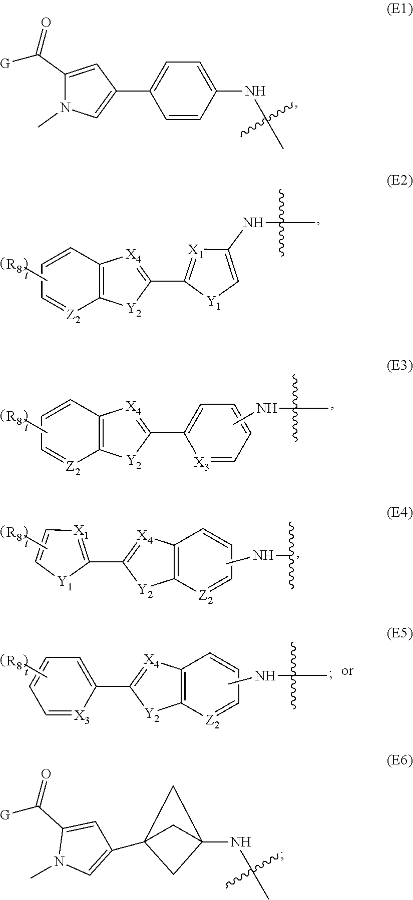

G is G1, G2, G3, G4, —OH, —NH—(C₁₋₆ alkylene)-R₁₃, —NR₁₃R₁₄, O—(CH₂)₃—NH₂, —O—CH(CH₃)—(CH₂)₂—NH₂ or —NH—(CH₂)₃—O—C(═O)—CH(CH₃)—NH₂:

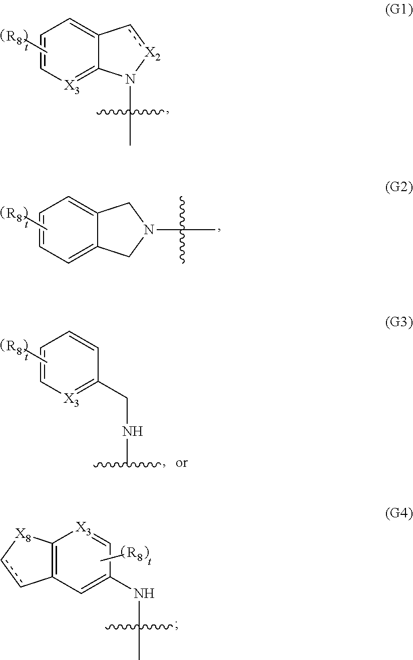

wherein the dotted line in G1 or G4 indicates the presence of a single or double bond;

each occurrence of R₂ and R₃ independently is an optionally substituted C₁₋₈ alkyl, optionally substituted C₂₋₈ alkenyl, optionally substituted C₂₋₈ alkynyl, optionally substituted C₃₋₈ cycloalkyl, optionally substituted 3- to 20-membered heterocycloalkyl, optionally substituted C₆₋₂₀ aryl or optionally substituted 5- to 20-membered heteroaryl, and, optionally in relation to the group NR₂R₃, R₂ and R₃ together with the nitrogen atom to which they are attached form an optionally substituted 4-, 5-, 6- or 7-membered heterocycloalkyl or an optionally substituted 5- or 6-membered heteroaryl;

R₄, R₅ and R₇ are each independently —H, —R₂, —OH, —OR₂, —SH, —SR₂, —NH₂, —NHR₂, —NR₂R₃, —NO₂, —SnMe₃, halo or a polyethylene glycol unit —(OCH₂CH₂)ᵣ—ORₐ; or R₄ and R₇ together form bis-oxy-C₁₋₃ alkylene;

each R₆ independently is —H, —R₂, —CO₂R₂, —COR₂, —CHO, —CO₂H, or halo;

each R₅ independently is —OH, halo, —NO₂, —CN, —N₃, —OR₂, —COH, —COOR₂, —COR₂, —OCONR₁₃R₁₄, —CONR₁₃R₁₄, —CO—NH—(C₁₋₆ alkylene)-R₁₃ₐ, C₁₋₁₀ alkyl, C₃₋₁₀ cycloalkyl, C₂₋₁₀ alkenyl, C₂₋₁₀ alkynyl, a polyethylene glycol unit —(OCH₂CH₂)ᵣ—ORₐ, 3- to 14-membered heterocycloalkyl, 5- to 12-membered heteroaryl, —S(═O)₂R₁₂, —S(═O)₂NR₁₃R₁₄, —SR₁₂, —SOₓM, —OSOₓM, —NR₉COR₁₉, —NH(C═NH)NH₂, —R₂₀-R₂₁—NR₁₃R₁₄, —R₂₀-R₂₁—NH—P(O)(OH)—(OCH₂CH₂)ₙ₉—OCH₃, or —O—P(O)(OH)—(OCH₂CH₂)ₙ₉—OCH₃;

each R₉ independently is C₁₋₁₀ alkyl, C₃₋₁₀ cycloalkyl, C₂₋₁₀ alkenyl or C₂₋₁₀ alkynyl;

R¹⁰ is —H or a nitrogen protecting group;

R¹¹ is -QR^{Q} or —SOₓM;

or R¹⁰ and R¹¹ taken together with the nitrogen atom and carbon atom to which they are respectively attached, form a N═C double bond;

each R₁₂ independently is C₁₋₇ alkyl, 3- to 20-membered heterocycloalkyl, 5- to 20-membered heteroaryl, or C₆₋₂₀ aryl;

each occurrence of R₁₃ and R₁₄ are each independently H, C₁₋₁₀ alkyl, 3- to 20-membered heterocycloalkyl, 5- to 20-membered heteroaryl, or C₆₋₂₀ aryl;

each R₁₃ₐ independently is —OH or —NR₁₃R₁₄;

R₁₅, R₁₆, R₁₇ and R₁₈ are each independently —H, —O, halo, —NO₂, —CN, —N₃, —OR₂, —COOH, —COOR₂, —COR₂, —OCONR₁₃R₁₄, C₁₋₁₀ alkyl, C₃₋₁₀ cycloalkyl, C₂₋₁₀ alkenyl, C₂₋₁₀ alkynyl, a polyethylene glycol unit —(OCH₂CH₂)ᵣ—ORₐ, 3-14 membered heterocycloalkyl, 5- to 12-membered heteroaryl, —NR₁₃R₁₄, —S(═O)₂R₁₂, —S(═O)₂NR₁₃R₁₄, —SR₁₂, —SOₓM, —OSOₓM, —NR₉COR₁₉ or —NH(C═NH)NH₂;

each R₁₉ independently is C₁₋₁₀ alkyl, C₃₋₁₀ cycloalkyl, C₂₋₁₀ alkenyl or C₂₋₁₀ alkynyl;

each R₂₀ independently is a bond, C₆₋₁₀ arylene, 3-14 membered heterocycloalkylene or 5- to 12-membered heteroarylene;

each R₂₁ independently is a bond or C₁₋₁₀ alkylene;

R₃₁, R₃₂ and R₃₃ are each independently —H, C₁₋₃ alkyl, C₂₋₃ alkenyl, C₂₋₃ alkynyl or cyclopropyl, wherein the total number of carbon atoms in the R₁ group is no more than 5;

R₃₄ is —H, C₁₋₃ alkyl, C₂₋₃ alkenyl, C₂₋₃ alkynyl, cyclopropyl, or phenyl wherein the phenyl is optionally substituted by one or more of halo, methyl, methoxy, pyridyl or thiophenyl;

one of R₃₅ₐ and R_{35b} is —H and the other is a phenyl group optionally substituted with one or more of halo, methyl, methoxy, pyridyl or thiophenyl;

R₃₆ₐ, R_{36b}, R_{36c} are each independently —H or C₁₋₂ alkyl;

R₃₆ₐ is —OH, —SH, —COOH, —C(O)H, —N═C═, —NHNH₂, —CONHNH₂,

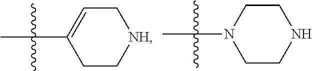 [SMILES: *C1=CCNCC1 ; *N1CCNCC1]

or NHR^{N}, wherein R^{N} is —H or C₁₋₄ alkyl;

R₃₇ₐ and R_{37b} are each independently is —H, —F, C₁₋₄ alkyl, C₂₋₃ alkenyl, wherein the alkyl and alkenyl groups are optionally substituted by C₁₋₄ alkyl amido or C₁₋₄ alkyl ester; or when one of R₃₇ₐ and R_{37b} is —H, the other is —CN or a C₁₋₄ alkyl ester;

R₃₈ and R₃₉ are each independently H, R₁₃, ═CH₂, ═CH—(CH₂)ₛ₁—CH₃, ═O, (CH₂)ₛ₁—OR₃, (CH₂)ₛ₁—CO₂R₁₃, (CH₂)ₛ₁—NR₁₃R₁₄, O—(CH₂)₂—NR₁₃R₁₄, NH—C(O)—R₃, O—(CH₂)ₛ₁—NH—C(O)—R₃, O—(CH₂)s-C(O)NHR₁₃, (CH₂)ₛ₁0S(═O)₂R₃, O—SO₂R₁₃, (CH₂)ₛ₁—C(O)R₁₃ and (CH₂)ₛ₁—C(O)NR₁₃R₁₄;

X₀ is CH₂, NR₆, C═O, BH, SO or SO₂;

Y₀ is O, CH₂, NR₆ or S;

Z₀ is absent or (CH₂)ₙ;

each X₁ independently is CR_{b}, or N;

each Y₁ independently is CH, NRₐ, O or S;

each Z₁ independently is CH, NRₐ, O or S;

each Rₐ independently is H or C₁₋₄ alkyl;

each R_{b} independently is H, OH, C₁₋₄ alkyl, or C₁₋₄ alkoxyl;

X₂ is CH, CH₂ or N;

X₃ is CH or N;

X₄ is NH, O or S;

X₈ is NH, O or S;

Q is O, S or NH;

when Q is S or NH, then R^{Q} is —H or optionally substituted C₁₋₂ alkyl; or

when Q is O, then R^{Q} is —H or optionally substituted C₁₋₂ alkyl, —SOₓM, —PO₃M, —(CH₂—CH₂—O)ₙ₉CH₃, —(CH₂—CH₂O)ₙ₉—(CH₂)₂—R₄₀, —C(O)—(CH₂—CH₂—O)ₙ₉CH₃, —C(O)O—(CH₂—CH₂—O)ₙ₉CH₃, —C(O)NH—(CH₂—CH₂—O)ₙ₉CH₃, —(CH₂)ₙ—NH—C(O)—CH₂—O—CH₂—C(O)—NH—(CH₂—CH₂—O)ₙ₉CH₃, —(CH₂)ₙ—NH—C(O)—(CH₂)ₙ—(CH₂—CH₂—O)ₙ₉CH₃, a sugar moiety,

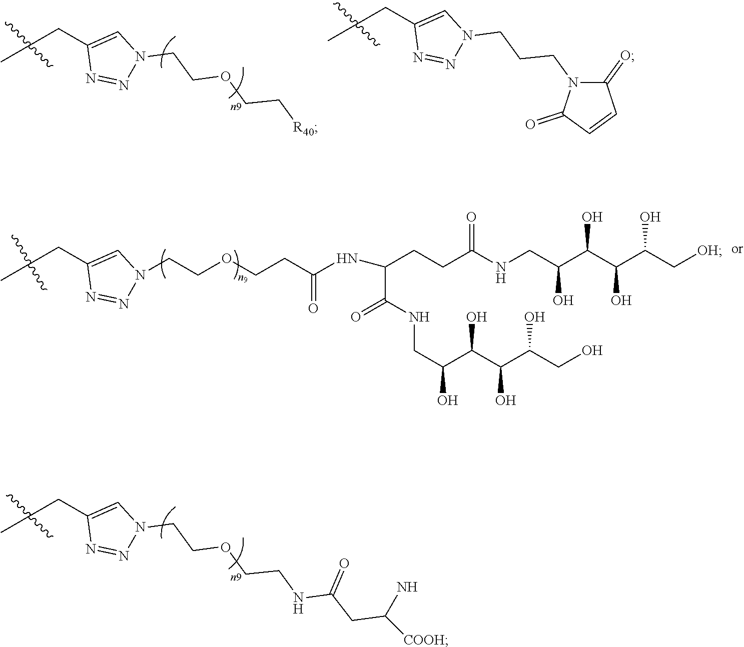

each M independently is H or a monovalent pharmaceutically acceptable cation;

n is 1, 2 or 3;

n₉ is 1, 2, 3, 4, 5, 6, 8, 12 or 24:

each r independently is an integer from 1 to 200;

s is 1, 2, 3, 4, 5 or 6;

s₁ is 0, 1, 2, 3, 4, 5 or 6;

t is 0, 1, or 2;

R₄₀ is —SO₃H, —COOH, —C(O)NH(CH₂)₂SO₃H, or —C(O)NH(CH₂)₂COOH; and

each x independently is 2 or 3.

In some embodiments, when D is

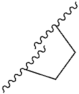 [SMILES: *CC*]

and s is 0, and T is —(CH₂)_{3 or 4}—, then E is not E3 wherein X₄ is N, Y₂ is O or S, Z₂ is CH, t is 0, 1, or 2, and R₈ is fluoro.

In some embodiments, when s is 1 and E is E3, then t is not 0, and R₈ is not C₁₋₄ alkyl, —C(O)—O—C₁₋₄alkyl, 3- to 14-membered heterocycloalkyl, or —O—(CH₂)₁₋₄-(3- to 14-membered heterocycloalkyl).

In some embodiments, when s is 1 and E is E4 or E5 wherein X₄ is CH, Y₂ is O or S, and Z₂ is CH, then t is not 0, and R₅ is not C₁₋₄ alkyl, —C(O)—O—C₄ alkyl, 3- to 14-membered heterocycloalkyl, or —O—(CH₂)₁₄-(3- to 14-membered heterocycloalkyl).

In some embodiments, when s is 0, E is E1, and G is —NR₁₃R₁₄ wherein one of R₁ and R₁₄ is H, then the other is not a 5- to 9-membered heteroaryl or phenyl.

In some embodiments, when G is G4, in which the dotted line indicates the presence of a double bond, X₃ is CH, and X₈ is O or S, then s is 2, 3, 4, 5 or 6. In some embodiment, s is 2. In some embodiments, s is 3. In some embodiments, s is 4. In some embodiments, s is 5. In some embodiments, s is 6.

In some embodiments, when X₈ is O or S, then s is 2, 3, 4, 5 or 6. In some embodiment, s is 2. In some embodiments, s is 3. In some embodiments, s is 4. In some embodiments, s is 5. In some embodiments, s is 6.

In some embodiments, the PBD drug moiety (D) is of Formula (IV-a),

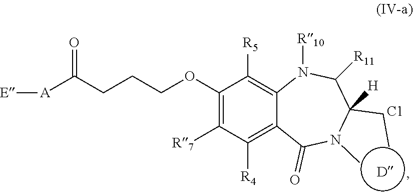

a tautomer thereof, a pharmaceutically acceptable salt or solvate thereof, or a pharmaceutically acceptable salt or solvate of the tautomer.

In some embodiments, D′ is D1.

In some embodiments, the PBD drug moiety (D) is of any one of formulae (V-1), (V-2), and (V-3):

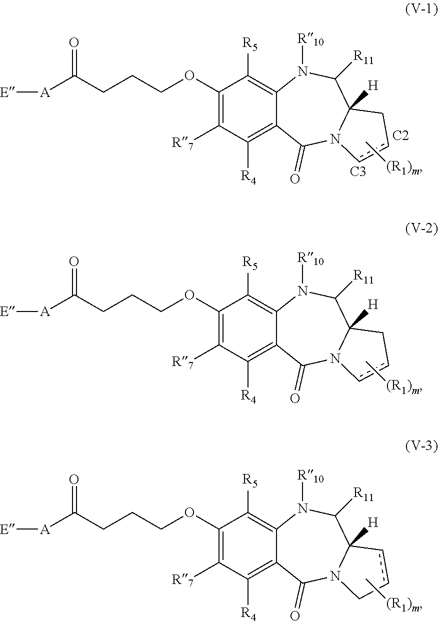

a tautomer thereof, a pharmaceutically acceptable salt or solvate thereof, or a pharmaceutically acceptable salt or solvate of the tautomer.

In some embodiments, D′ is D2.

In some embodiments, the PBD drug moiety (D) is of Formula (VI-1):

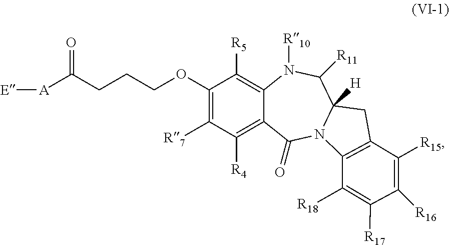

a tautomer thereof, a pharmaceutically acceptable salt or solvate thereof, or a pharmaceutically acceptable salt or solvate of the tautomer.

In some embodiments, D′ is D3 or D4.

In some embodiments, the PBD drug moiety (D) is of Formula (VII), (VII-1), (VII-2) or (VII-3):

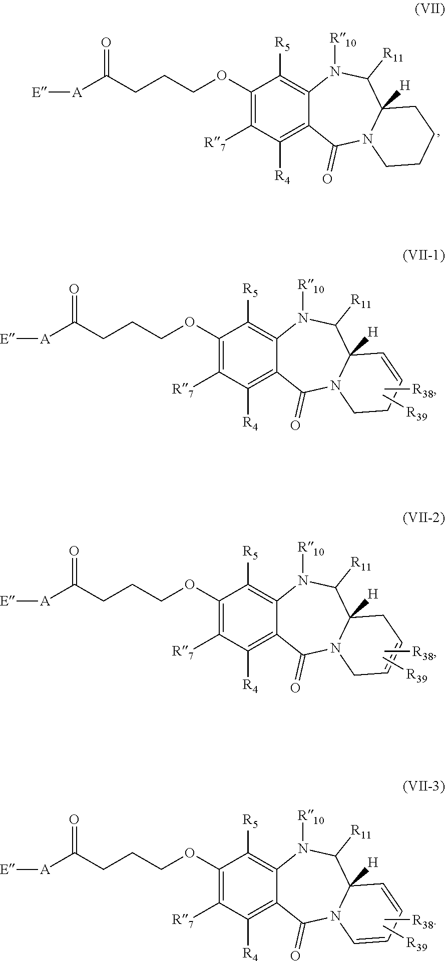

a tautomer thereof, a pharmaceutically acceptable salt or solvate thereof, or a pharmaceutically acceptable salt or solvate of the tautomer.

In some embodiments, the PBD drug moiety (D) is of Formula (VIII):

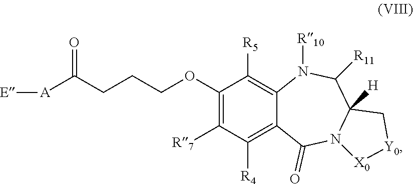

a tautomer thereof, a pharmaceutically acceptable salt or solvate thereof, or a pharmaceutically acceptable salt or solvate of the tautomer.

In some embodiments, T is C₂₋₄ alkylene linker.

In some embodiments, A is

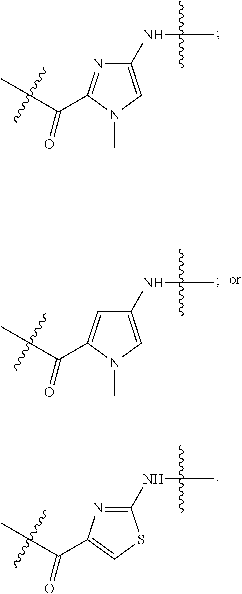 [SMILES: *Nc1cn(C)c(C(*)=O)n1 ; *Nc1cc(C(*)=O)n(C)c1 ; *Nc1nc(C(*)=O)cs1]

In some embodiments, A is

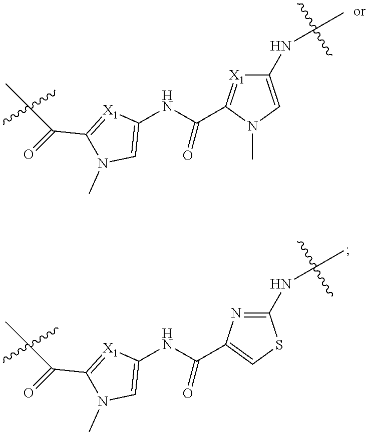

wherein each X₁ independently is CH or N.

In some embodiments, A is

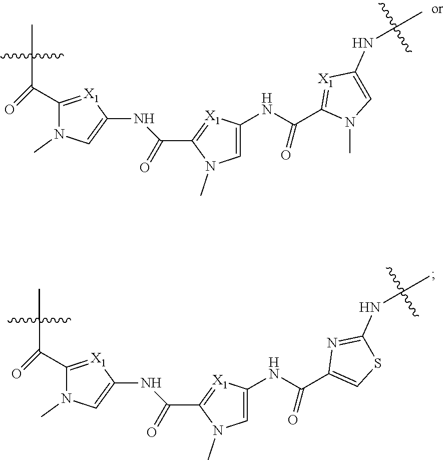

wherein each X₁ independently is CH or N.

In some embodiments, A is:

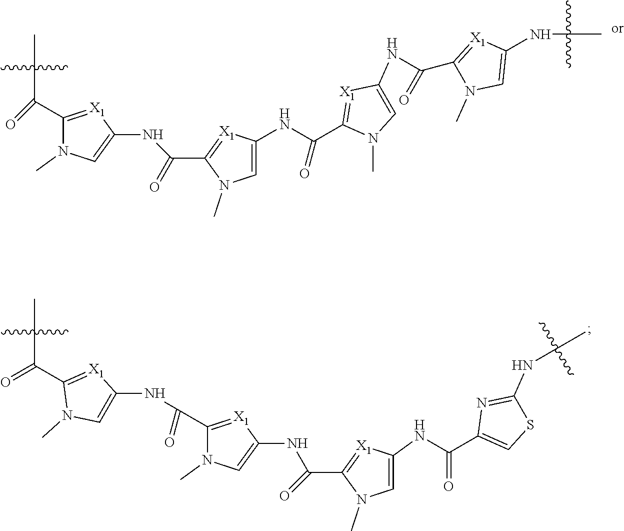

wherein each X₁ independently is CH or N.

In some embodiments, E is

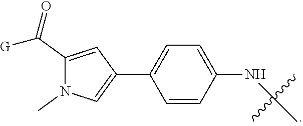

In some embodiments, E is

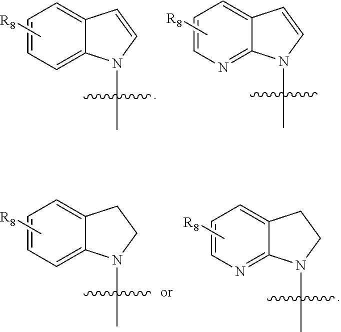

In some embodiments, the PBD drug moiety (D) is of Formulae (IX-a) to (IX-r).

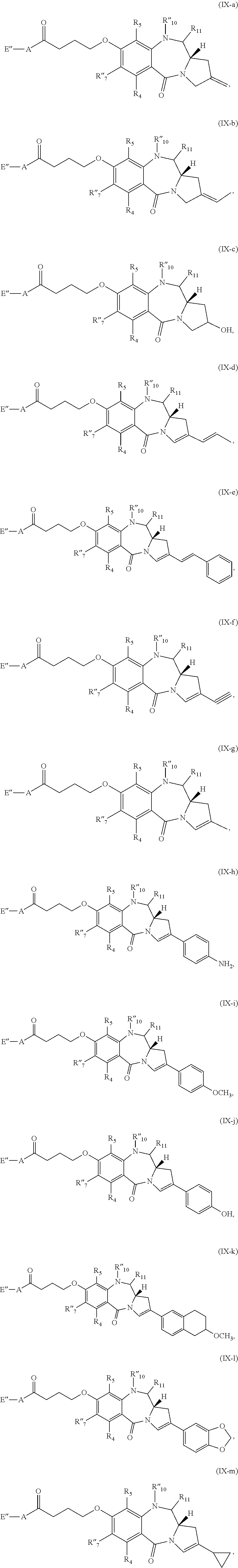

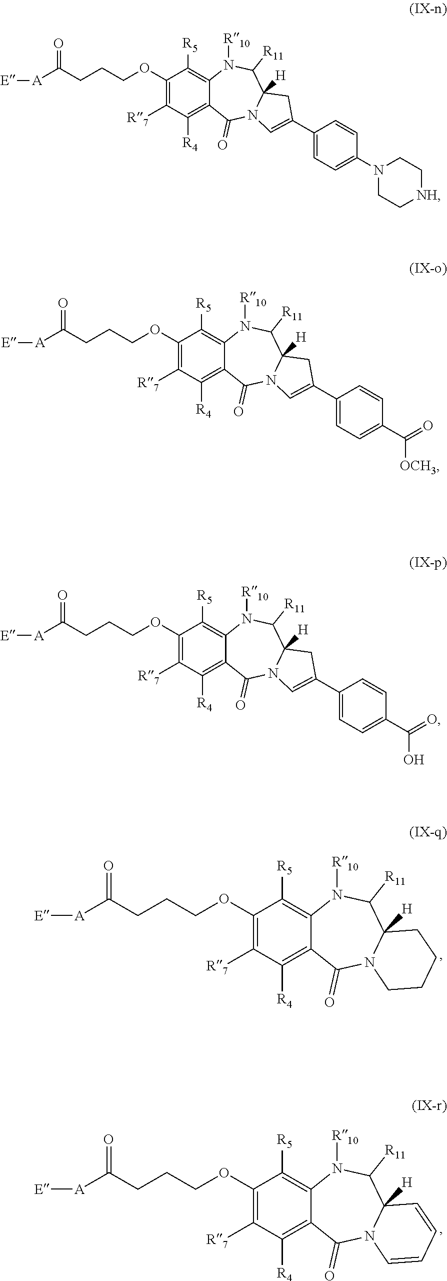

a tautomer thereof, a pharmaceutically acceptable salt or solvate thereof, or a pharmaceutically acceptable salt or solvate of the tautomer. In some embodiments, the PBD drug moiety (D), prior to being connected to another portion of the conjugate, corresponds to a compound selected from the compounds listed in Table 1, tautomers thereof, pharmaceutically acceptable salts or solvates thereof, or pharmaceutically acceptable salts or solvates of the tautomers.

In some embodiments, the PBD drug moiety (D), prior to being connected to another portion of the conjugate, corresponds to a compound of any one of Formula (XIIIa) to (XIIIm):

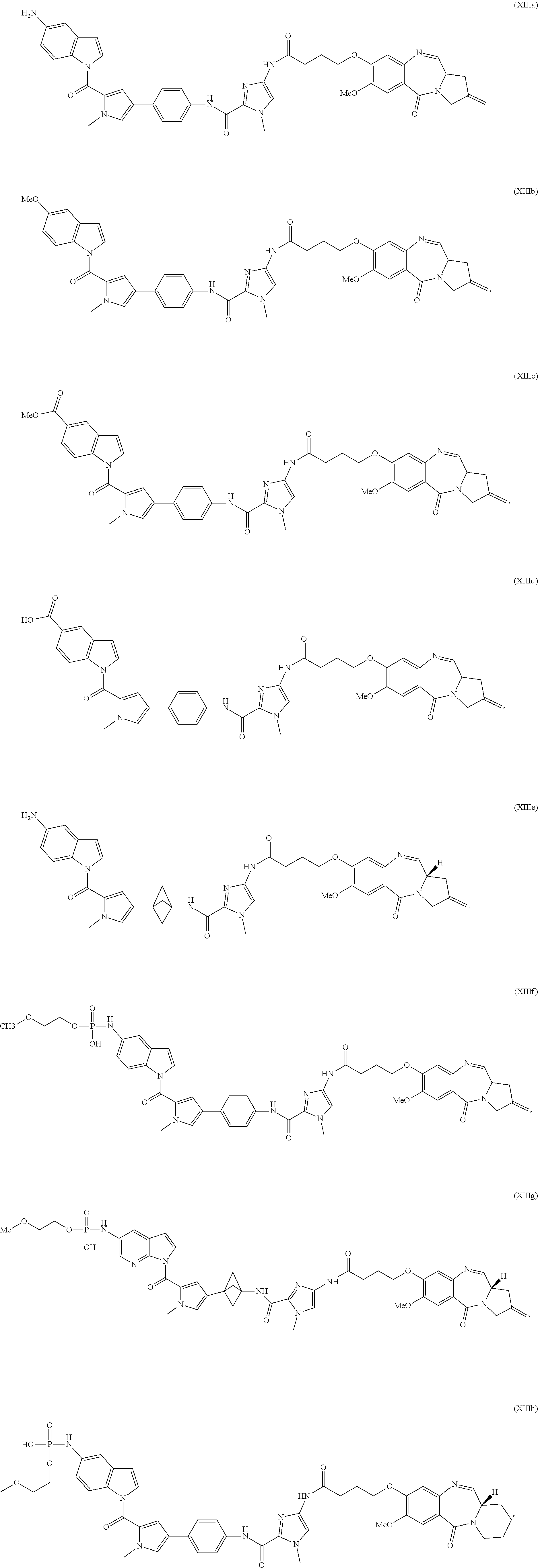

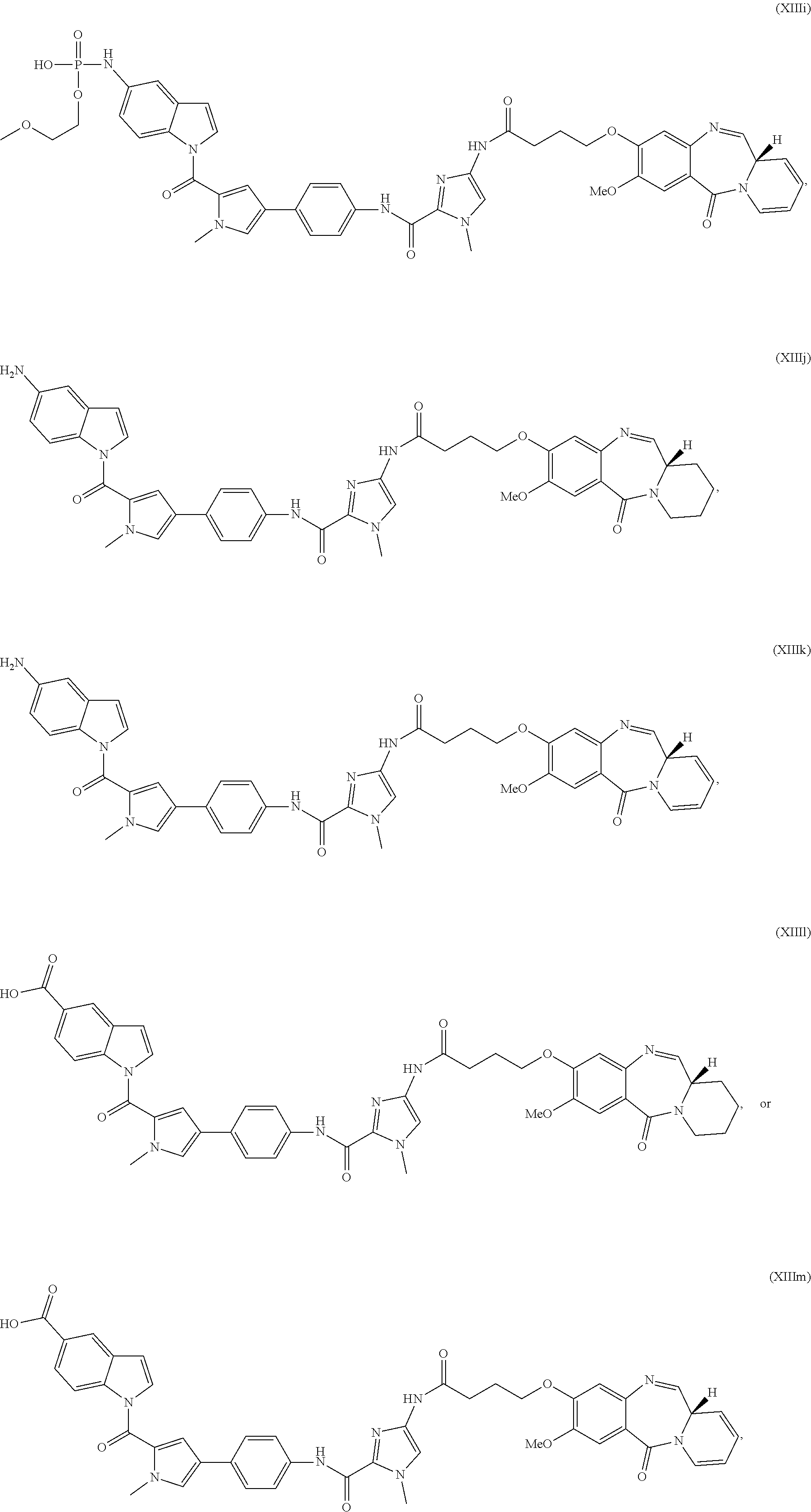

a tautomer thereof, a pharmaceutically acceptable salt or solvate thereof, or a pharmaceutically acceptable salt or solvate of the tautomer.

In some embodiments, the PBD drug moiety (D) is selected from the conjugates listed in Table 1A, tautomers thereof, pharmaceutically acceptable salts or solvates thereof, and pharmaceutically acceptable salts or solvates of the tautomers.

In some embodiments, the conjugate is selected from the conjugates listed in Table 2, tautomers thereof, pharmaceutically acceptable salts or solvates thereof, and pharmaceutically acceptable salts or solvates of the tautomers.

In some aspects, the present disclosure provides a pharmaceutical composition comprising the conjugate of any one of the preceding claims and a pharmaceutically acceptable carrier.

In some aspects, the present disclosure provides a method of treating or preventing a disease or disorder, comprising administering to a subject in need thereof a pharmaceutically effective amount of the conjugate of any one of the preceding claims.

In some embodiments, the disease or disorder is cancer.

In some aspects, the present disclosure provides a conjugate disclosed herein for use in treating or preventing a disease or disorder.

In some aspects, the present disclosure provides use of a conjugate disclosed herein in treating or preventing a disease or disorder.

In some aspects, the present disclosure provides use of a conjugate disclosed herein in the manufacture of a medicament for treating or preventing a disease or disorder.

Unless otherwise defined, all technical and scientific terms used herein have the same meaning as commonly understood by one of ordinary skill in the art to which this invention belongs. In the specification, the singular forms also include the plural unless the context clearly dictates otherwise. Although methods and materials similar or equivalent to those described herein can be used in the practice or testing of the present invention, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting.

Other features and advantages of the disclosure will be apparent from the following detailed description and claims.

### BRIEF DESCRIPTION OF THE FIGURES

FIG. 1 illustrates the anti-tumor efficacy of the Conjugate 10 at 1 mg/kg or at 3 mg/kg; as measured in a Calu-3 mouse tumor xenograft model.

FIG. 2 illustrates the anti-tumor efficacy of the Conjugate 10, Conjugate 26 and Conjugate 36 each at 1 mg/kg and at 3 mg/kg, and Conjugate 31, Conjugate 38 and Conjugate 46 at each 1 mg/kg; as measured in an Calu-3 mouse tumor xenograft model.

FIG. 3 illustrates the anti-tumor efficacy of Conjugate 61 and Conjugate 63 each at 1 mg/kg or at 3 mg/kg, and Conjugate 62 and Conjugate 64 each at 3 mg/kg; as measured in an DLD1 mouse tumor xenograft model.

FIG. 4 illustrates the anti-tumor efficacy of the Conjugate 135 at 1 mg/kg and at 3 mg/kg, Conjugate 135A at 2.2 mg/kg, Conjugate 136 at 2.2 mg/kg and 4.4 mg/kg, and Conjugate 136A at 3 mg/kg; as measured in an OVCAR-3 mouse tumor xenograft model.

FIG. 5 illustrates the anti-tumor efficacy of the Conjugate 10A at 3 mg/kg. as measured in HT-29 mouse tumor xenograft model

### DETAILED DESCRIPTION

In some aspects, the present disclosure provides, inter alia, a conjugate (e.g., an antibody-drug conjugate (ADC)) of Formula (I):

PBRM-[L^{C}-D]_{d15} (I)

or a pharmaceutically acceptable salt or solvate thereof, wherein:

PBRM denotes a protein based recognition-molecule;

L^{C} is a linker unit connecting the PBRM to D;

D is a PBD drug moiety; and

d₁₅ is an integer from about 1 to about 20.

In some embodiments, the conjugates of Formula (I) include those where each of the moieties defined for one of PBRM, L^{C}, D, and d₁₅ can be combined with any of the moieties defined for the others of PBRM, L^{C}, D, and d₁₅.

In some embodiments, the PBRM is a targeting agent that binds to a target moiety. In some embodiments, the PBRM is a cell binding agent specifically binding to a cell component. In some embodiments, the PBRM specifically binds to a target molecule of interest.

In some embodiments, the conjugate allows for delivery of the PBD drug moiety (D) to a preferred site in a subject (e.g., a human). In some embodiments, the conjugate allows for the release of the PBD drug moiety (D) in an active form for its intended therapeutic effect.

In some embodiments, the conjugate comprises the PBD drug moiety (D) being covalently attached to a cell binding agent via the linker unit (L).

In some embodiments, the linker unit is a bifunctional or multifunctional moiety which being capable of linking one or more PBD drug moiety (D) and an antibody unit (Ab) to form an antibody-drug conjugate (ADC). The linker unit may be stable outside a cell (i.e., extracellularly), or it may be cleavable by enzymatic activity, hydrolysis, or other metabolic conditions.

In some embodiments, the linker unit of the ADC prevents aggregation of the ADC and/or keep the ADC freely soluble in aqueous media and in a monomeric state.

In some embodiments, the linker unit of the ADC is stable extracellularly. In some embodiments, before transport or delivery into a cell, the ADC is preferably stable and remains intact (i.e., the antibody remains linked to the drug moiety). In some embodiments, the linker unit is stable outside the target cell and may be cleaved at an efficacious rate inside the cell. For example, the linker unit may (i) maintain the specific binding properties of the antibody; (ii) allow for intracellular delivery of the conjugate or therapeutic agent; (iii) remain stable and intact (i.e., not cleaved) until the conjugate has been delivered or transported to its targeted site; and/or (iv) maintain a cytotoxic, cell-killing effect or a cytostatic effect of the PBD drug moiety. Stability of the ADC may be measured by standard analytical techniques such as mass spectroscopy, HPLC, and the separation/analysis technique LC/MS.

Covalent attachment of the antibody and the PBD drug moiety requires the linker unit to have two reactive functional groups (i.e., bivalency in a reactive sense). Useful bivalent linker units for attaching two or more functional or biologically active moieties include, but are not limited to, peptides, nucleic acids, drugs, toxins, antibodies, haptens, and reporter groups. Some known bivalent linker units and their resulting conjugates have been described (Hermanson, G. T. (1996) Bioconjugate Techniques; Academic Press: New York, p 234-242).

In some embodiments, the linker unit may be substituted with one or more groups which modulate aggregation, solubility, and/or reactivity. In some embodiments, a sulfonate substituent may increase water solubility of the reagent and facilitate the coupling reaction of the linker reagent with the antibody or the PBD drug moiety, or facilitate the coupling reaction of an antibody-linker reagent (Ab-L) with a PBD drug moiety (D), or a PBD drug-linker reagent (D-L) with an antibody unit (Ab), depending on the synthetic route employed to prepare the ADC.

In some aspects, the present disclosure provides a method of preparing a conjugate (e.g., an antibody-drug conjugate (ADC)) of the present disclosure. Antibody-drug conjugates (ADC) can be conveniently prepared using a linker unit having reactive functionality for binding to the PBD drug moiety (D) and to the antibody unit (Ab). In some embodiments, a cysteine thiol, or an amine (e.g. N-terminus or amino acid side chain such as lysine) of the antibody (Ab) can form a bond with a functional group of a linker or spacer reagent, a PBD drug moiety (D), or a PBD drug-linker reagent (D-RL).

### Antibody-Drug Conjugate (ADC) Type I:

In some embodiments, the conjugate (e.g., the antibody-drug conjugate (ADC)) of the present disclosure is of Formula (II):

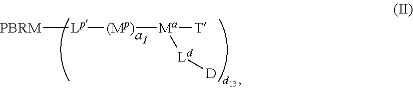

or a pharmaceutically acceptable salt or solvate thereof,
wherein:

PBRM denotes a protein based recognition-molecule;

each occurrence of D is independently a PBD drug moiety;

L^{P′} is a divalent linker moiety connecting the PBRM to M^{P}; of which the corresponding monovalent moiety L^{P} contains a functional group W^{P} that is capable of forming a covalent bond with a functional group of the PBRM;

M^{P} is a Stretcher unit;

a₁ is an integer from 0 to 1;

M^{A} comprises a peptide moiety that contains at least two amino acids;

T′ is a hydrophilic group and the

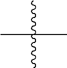

between T′ and M^{A} denotes direct or indirect attachment of T′ and M^{A};

each occurrence of L^{D} is independently a divalent linker moiety connecting D to M^{A} and comprises at least one cleavable bond such that when the bond is broken, D is released in an active form for its intended therapeutic effect; and

d₁₃ is an integer from 1 to 14.

In some embodiments, the conjugates of Formula (II) include those where each of the moieties defined for one of PBRM, D, L^{P′}, L^{P}, W^{P}, M^{P}, a₁, M^{A}, T′, L^{D}, and d₁₃ can be combined with any of the moieties defined for the others of PBRM, D, L^{P′}, L^{P}, W^{P}, M^{P}, a₁, M^{A}, T′, L^{D}, and d₁₃.

In some aspects, the present disclosure provides a scaffold of any one of Formulae (Ha) to (IIe):

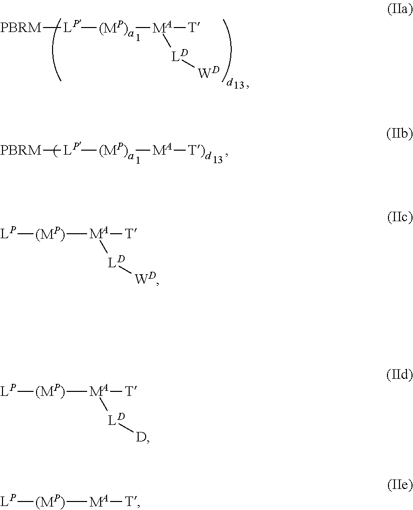

or a pharmaceutically acceptable salt or solvate thereof, wherein

PBRM denotes a protein based recognition-molecule;

each occurrence of D is independently a PBD drug moiety;

L^{P′} is a divalent linker moiety connecting the PBRM to M^{P}; of which the corresponding monovalent moiety L^{P} contains a functional group W^{P} that is capable of forming a covalent bond with a functional group of the PBRM:

M^{P} is a Stretcher unit;

a₁ is an integer from 0 to 1; M^{A} comprises a peptide moiety that contains at least two amino acids;

T′ is a hydrophilic group and the

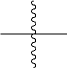

between T′ and M^{A} denotes direct or indirect attachment of T′ and M^{A};

each occurrence of W^{D} is independently a functional group that is capable of forming a covalent bond with a functional group of D; each occurrence of L^{D} is independently a divalent linker moiety connecting W^{D} or D to M^{A} and L^{D} comprises at least one cleavable bond such that when the bond is broken, D is released in an active form for its intended therapeutic effect; and

d₁₃ is an integer from 1 to 10.

In some embodiments, the conjugates of any one of Formulae (IIa)-(IIe) include those where each of the moieties defined for one of PBRM, D, L^{P′}, L^{P}, W^{P}, M^{P}, a₁, M^{A}, T′, L^{D}, W^{D}, and d₁₃ can be combined with any of the moieties defined for the others of PBRM, D, L^{P′}, L^{P}, W^{P}, M^{P}, a₁, M^{A}, T′, L^{D}, W^{D}, and d₁₃.

The conjugates and scaffolds of the disclosure can include one or more of the following features when applicable.

In some embodiments, d₁₃ is an integer from 2 to 14, from 2 to 12, from 2 to 10, from 2 to 8, from 2 to 6, from 2 to 4, from 4 to 10, from 4 to 8, from 4 to 6, from 6 to 14, from 6 to 12, from 6 to 10, from 6 to 8, from 8 to 14, from 8 to 12, or from 8 to 10.

In some embodiments, d₁₃ is an integer from 2 to 6 (e.g., d₁₃ is 2, 3, 4, 5 or 6).

In some embodiments, d₁₃ is an integer from 2 to 4 (e.g., d₁₃ is 2, 3, or 4).

In some embodiments, d₁₃ is an integer from 4 to 6 (e.g., d₁₃ is 4, 5, or 6).

In some embodiments, do is an integer from 6 to 8 (e.g., d₃ is 6, 7, or 8).

In some embodiments, d₁₃ is an integer from 6 to 10 (e.g., d₁₃ is 6, 7, 8, 9, or 10).

In some embodiments, d₁₃ is 3 to 5.

In some embodiments, d₁₃ is 4 or 5.

L^{P} and L^{P′}

In some embodiments, L^{P′} is a divalent linker moiety connecting the PBRM to M^{P}; of which the corresponding monovalent moiety is L^{P}.

In some embodiments, L^{P}, when not connected to PBRM, comprises a terminal group W^{P} in which each W^{P} independently is:

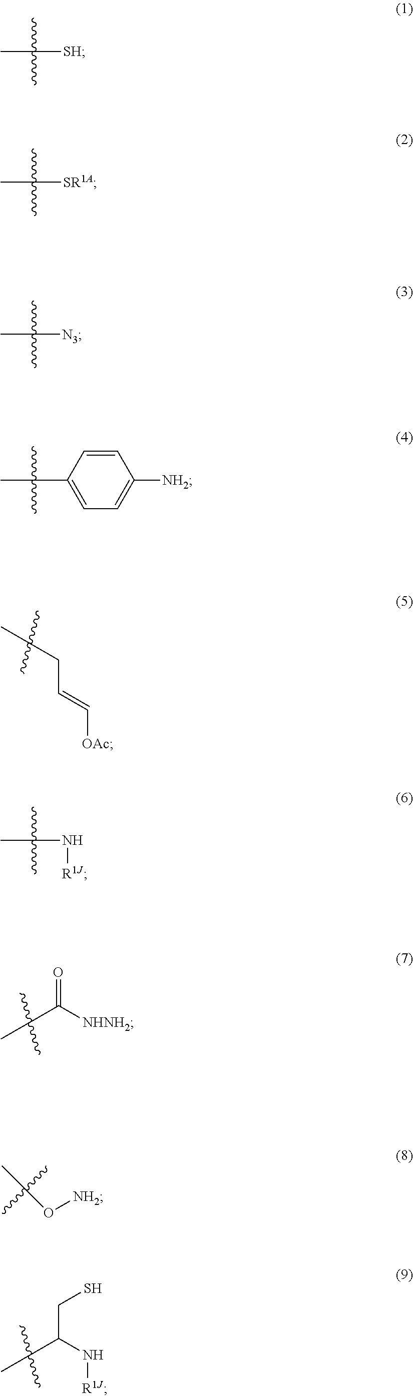

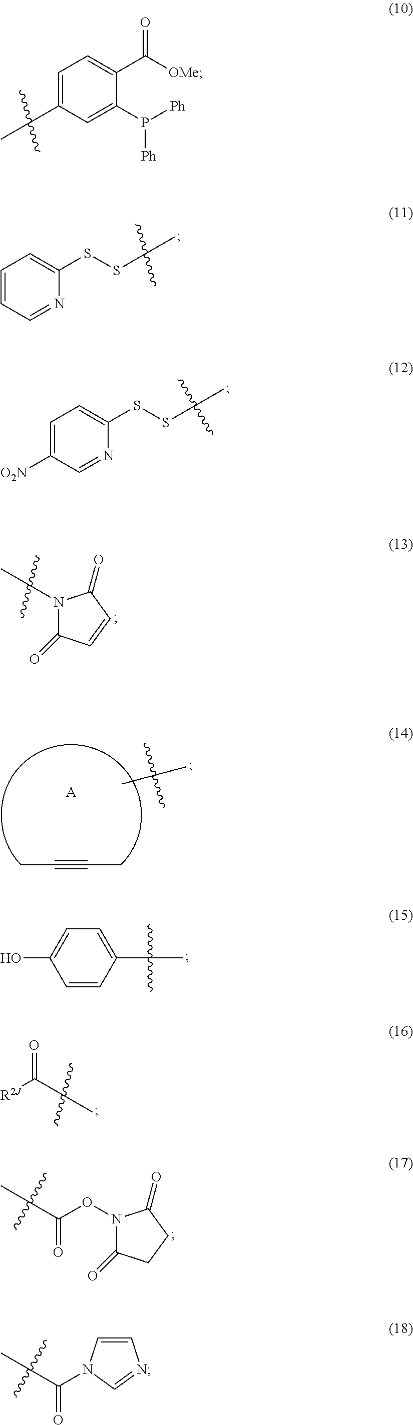

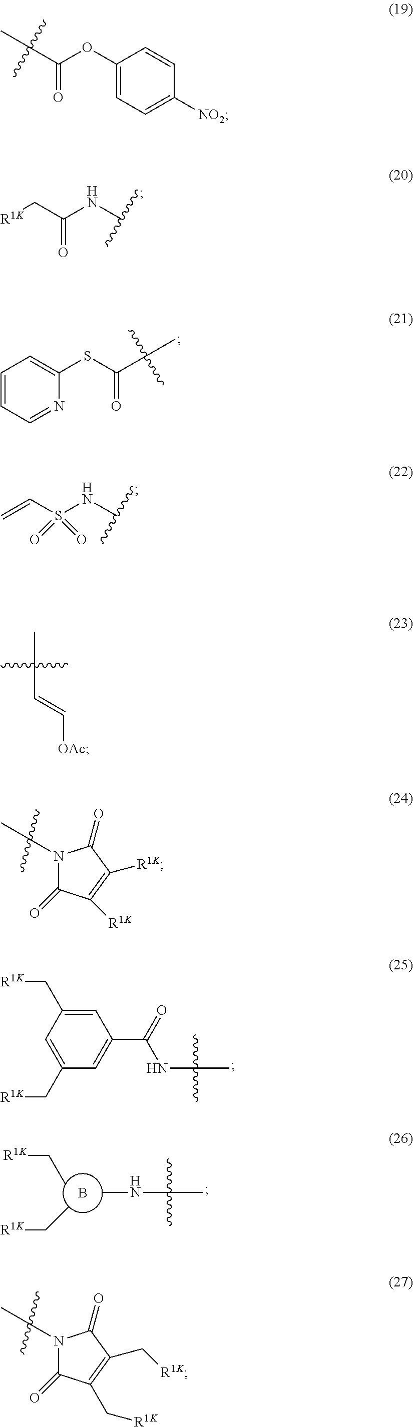

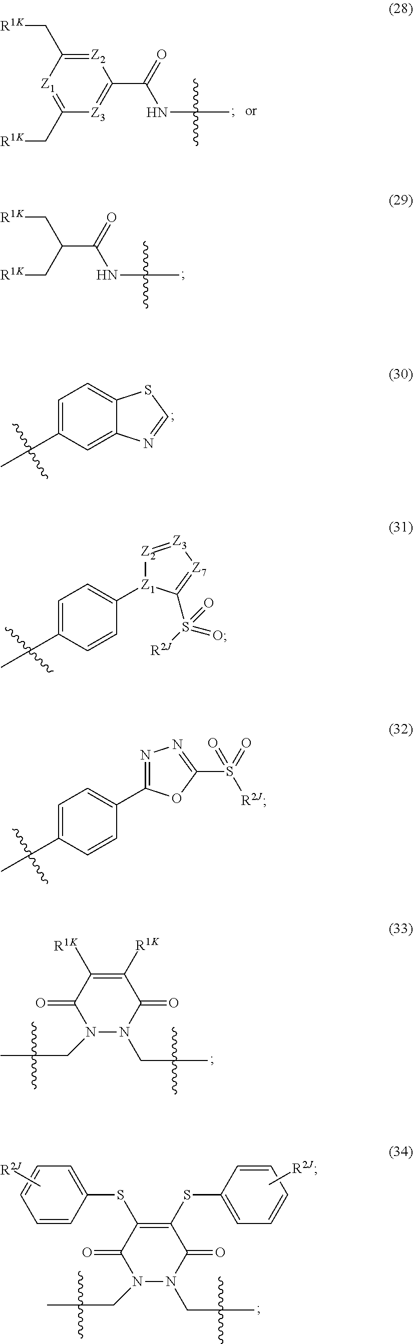

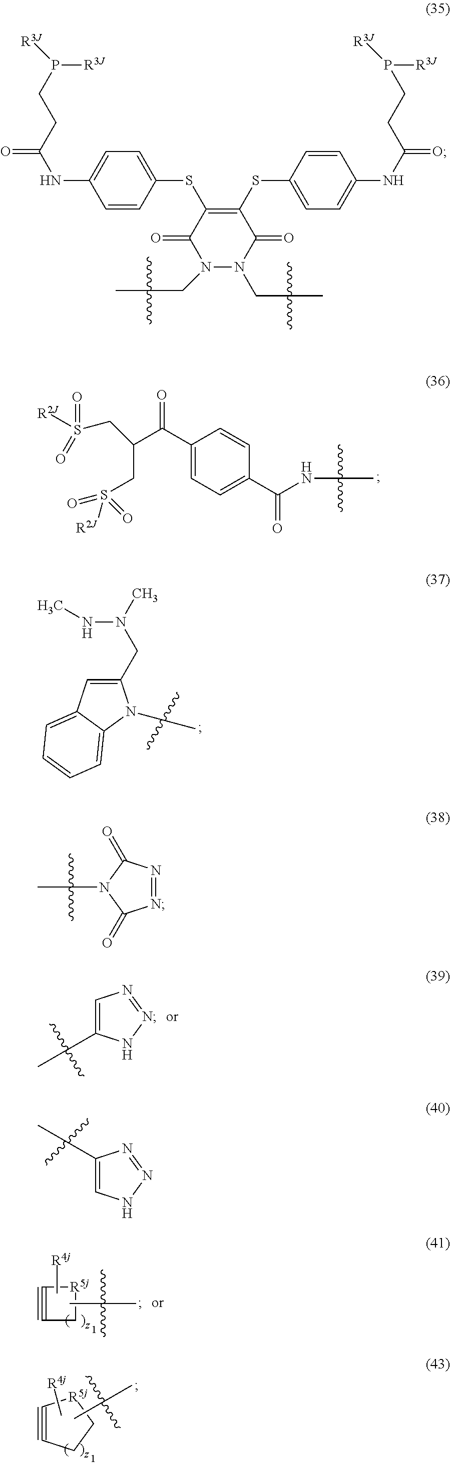

in which

R^{1K} is a leaving group (e.g., halide or RC(O)O— in which R is hydrogen or an aliphatic, heteroaliphatic, carbocyclic, or heterocycloalkyl moiety);

R^{1A} is a sulfur protecting group;

ring A is cycloalkyl or heterocycloalkyl;

ring B is cycloalkyl, heterocycloalkyl, aryl, or heteroaryl;

R^{1J} is hydrogen or an aliphatic, heteroaliphatic, carbocyclic, or heterocycloalkyl moiety;

R^{2J} is hydrogen or an aliphatic, aryl, heteroaliphatic, or carbocyclic moiety;

R^{3J} is C₁₋₆ alkyl; Z₁, Z₂, Z₃ and Z₇ are each independently a carbon or nitrogen atom;

R^{4j} is hydrogen, halogen, OR, —NO₂, —CN, —S(O)₂R, C₁₋₂₄ alkyl (e.g., C₁₋₆alkyl), or 6-24 membered aryl or heteroaryl, wherein the C₁₋₂₄ alkyl (e.g., C₁₋₆alkyl), or 6-24 membered aryl or heteroaryl, is optionally substituted with one or more aryl or heteroaryl; or two R^{4j} together form an annelated cycloalkyl, heterocycloalkyl, aryl, or heteroaryl; R is hydrogen or an alkyl, heteroalkyl, cycloalkyl, or heterocycloalkyl moiety;

R^{5j} is C(R^{4j})₂, O, S or NR; and

z₁ is an integer 1, 2, 3, 4, 5, 6, 7, 8, 9 or 10.

In some embodiments, each R^{1K} is halo or RC(O)O— in which R is hydrogen or an aliphatic, heteroaliphatic, carbocyclic, or heterocycloalkyl moiety.

In some embodiments, each R^{1A} independently is

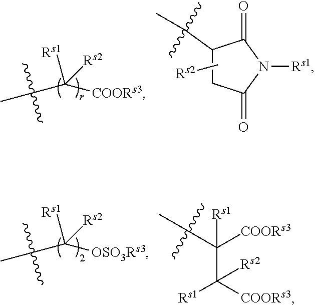

in which r is 1 or 2 and each of R^{s1}, R^{s2}, and R^{s3} is hydrogen, or an aliphatic, heteroaliphatic, carbocyclic, or heterocycloalkyl moiety.

In some embodiments, ring A is C₃₋₈ cycloalkyl or 5-19 membered heterocycloalkyl.

In some embodiments, ring A is

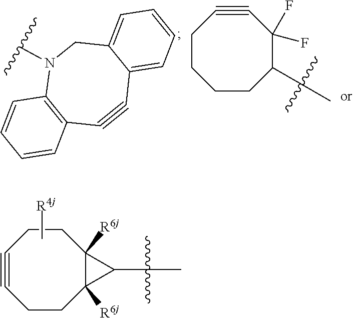

wherein R^{6j} is hydrogen, halogen, C₁₋₂₄ alkyl (e.g., C₁₋₆ alkyl), or 6-24 membered aryl or heteroaryl, wherein the C₁₋₂₄ alkyl (e.g., C₁₋₆ alkyl), or 6-24 membered aryl or heteroaryl, is optionally substituted with one or more aryl or heteroaryl.

In some embodiments, ring A is

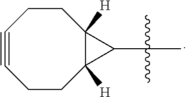 [SMILES: *C1[C@H]2CCC#CCC[C@@H]12]

In some embodiments, ring A or B is C₃₋₈ cycloalkyl or 3-12 membered heterocycloalkyl.

In some embodiments, ring A or B is piperazinyl or piperidinyl.

In some embodiments, each of R^{s1}, R^{s2}, and R^{s3} is hydrogen or C₁₋₆ alkyl.

In some embodiments, W^{P} is

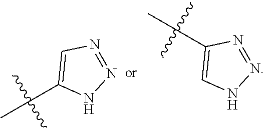 [SMILES: *c1cnn[nH]1 ; *c1c[nH]nn1]

In some embodiments, W^{P} is

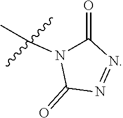 [SMILES: *N1C(=O)N=NC1=O]

In some embodiments, when W^{P} is

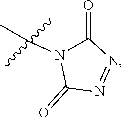 [SMILES: *N1C(=O)N=NC1=O]

L^{P′} comprises

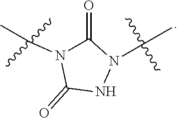 [SMILES: *n1[nH]c(=O)n(*)c1=O]

In some embodiments, W^{P} is

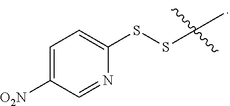 [SMILES: *SSc1ccc([N+](=O)[O-])cn1]

In some embodiments. W^{P} is

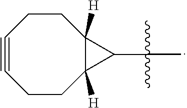 [SMILES: *C1[C@H]2CCC#CCC[C@@H]12]

In some embodiments, W^{P} is

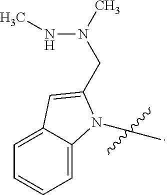 [SMILES: *n1c(CN(C)NC)cc2ccccc21]

In some embodiments. W^{P} is

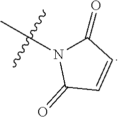 [SMILES: *N1C(=O)C=CC1=O]

In some embodiments, when W^{P} is

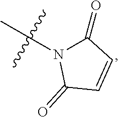 [SMILES: *N1C(=O)C=CC1=O]

L^{P′} comprises

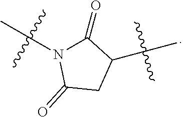 [SMILES: *C1CC(=O)N(*)C1=O]

In some embodiments, W^{P} is

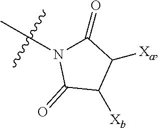

wherein one of Xₐ and X_{b} is H and the other is a maleimido blocking moiety. In some embodiments, a maleimido blocking compound (i.e., a compound that can react with maleimide to convert it to succinimide) may be used to quench the reaction between, e.g., the Linker-Drug moiety and PBRM, and a maleimido blocking moiety refers to the chemical moiety attached to the succinimide upon conversion. In some embodiments, the maleimido blocking moieties are moieties that can be covalently attached to one of the two olefin carbon atoms upon reaction of the maleimido group with a thiol-containing compound of Formula (II′):

R₉₀—(CH₂)_{d}—SH (II′)

wherein:

R₉₀ is NHR₉₁, OH, COOR₉₃, CH(NHR₉₁)COOR₉₃ or a substituted phenyl group;

R₉₃ is hydrogen or C₁₋₄ alkyl;

R₉₁ is hydrogen, CH₃ or CH₃CO and

d is an integer from 1 to 3.

In some embodiments, the maleimido blocking compound is cysteine, N-acetyl cysteine, cysteine methyl ester, N-methyl cysteine, 2-mercaptoethanol, 3-mercaptopropanoic acid, 2-mercaptoacetic acid, mercaptomethanol (i.e., HOCH₂SH), benzyl thiol in which phenyl is substituted with one or more hydrophilic substituents, or 3-aminopropane-1-thiol. The one or more hydrophilic substituents on phenyl comprise OH, SH, methoxy, ethoxy, COOH CHO, COC₁₋₄ alkyl, NH₂, F, cyano, SO₃H, PO₃H, and the like.

In some embodiments, the maleimido blocking group is —S—(CH₂)_{d}—R₉₀, wherein:

R₉₀ is OH, COOH, or CH(NHR₉₁)COOR₉₃;

R₉₃ is hydrogen or CH₃;

R₉₁ is hydrogen or CH₃CO; and

d is 1 or 2.

In some embodiments, the maleimido blocking group is —S—CH₂—CH(NH₂)COOH.

### Stretcher Unit M^{P}

In some embodiments, M^{P}, when present, is —(Z₄)—[(Z₅)—(Z₆)]_{z}—, with Z₄ connected to L^{P′} or L^{P} and Z₆ connected to M^{A}; in which

z is 1, 2, or 3;

Z₄ is:

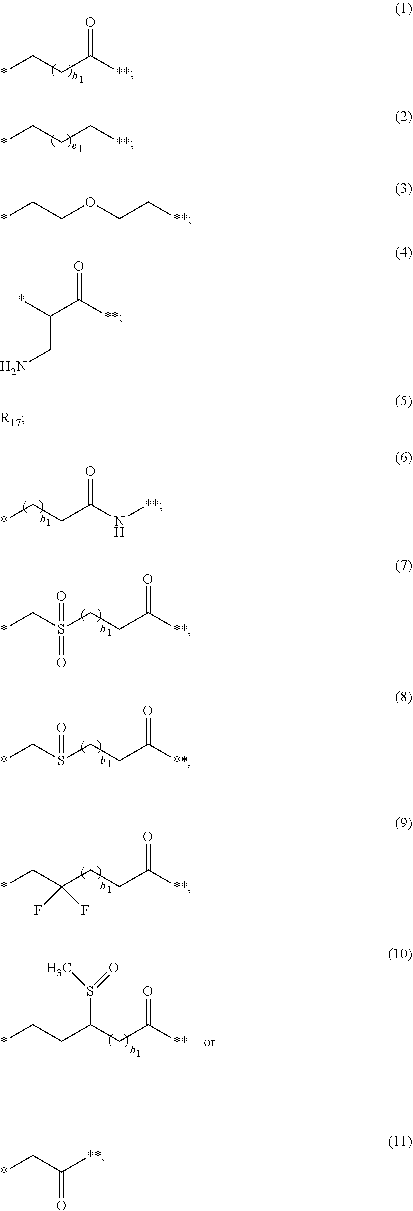

wherein * denotes attachment to L^{P′} or L^{P} and ** denotes attachment to Z₅ or Z₆ when present or to M^{A} when Z₅ and Z₆ are both absent;
b₁ is an integer from 0 to 6;
e₁ is an integer from 0 to 8,

R₁₇ is C₁₋₁₀ alkylene, C₁₋₁₀ heteroalkylene, C₃₋₈ cycloalkylene, O—(C₁₋₈ alkylene, arylene, —C₁₋₁₀ alkylene-arylene-, -arylene-C₁₋₁₀ alkylene-, —C₁₋₁₀ alkylene-(C₃₋₈ cycloalkylene)-, —(C₃₋₈ cycloalkylene —C₁₋₁₀ alkylene-, 4 to 14-membered heterocycloalkylene, —C₁₋₁₀ alkylene-(4 to 14-membered heterocycloalkylene)-, -(4 to 14-membered heterocycloalkylene)-C₁₋₁₀ alkylene-, —C₁₋₁₀ alkylene-C(═O)—, —C₁₋₁₀ heteroalkylene-C(═O)—, —C₃₋₈ cycloalkylene-C(═O)—, —O—(C₁₋₈ alkyl)-C(═O)—, -arylene-C(═O)—, —C₁₋₁₀ alkylene-arylene-C(═O)—, -arylene —C₁₋₁₀ alkylene-C(═O)—, —C₁₋₁₀ alkylene-(C₃₋₈ cycloalkylene)-C(═O)—, —(C₃₋₈ cycloalkylene)-C₁₋₁₀ alkylene-C(═O)—, -4 to 14-membered heterocycloalkylene-C(═O)—, —C₁₋₁₀ alkylene-(4 to 14-membered heterocycloalkylene)-C(═O)—, -(4 to 14-membered heterocycloalkylene)-C₁₋₁₀ alkylene-C(═O)—, —C₁₋₁₀ alkylene-NH—, —C₁₋₁₀ heteroalkylene-NH—, —C₃₋₈ cycloalkylene-NH—, —O—(C₁₋₈ alkyl)-NH—, -arylene-NH—, —C₁₋₁₀ alkylene-arylene-NH—, -arylene-C₁₋₁₀ alkylene-NH—, —C₁₋₁₀ alkylene-(C₃₋₈ cycloalkylene)-NH—, —(C₃₋₈ cycloalkylene)-C₁₋₁₀ alkylene-NH—, -4 to 14-membered heterocycloalkylene-NH—, —C₁₋₁₀ alkylene-(4 to 14-membered heterocycloalkylene)-NH—, -(4 to 14-membered heterocycloalkylene)-C₁₋₁₀ alkylene-NH—, —C₁₋₁₀ alkylene-S—, —C₁₋₁₀ heteroalkylene-S—, —C₃₋₈ cycloalkylene-S—, —O—C₁₋₈ alkyl)-S—, -arylene-S—, —C₁₋₁₀ alkylene-arylene-S—, -arylene-C₁₋₁₀ alkylene-S—, —C₁₋₁₀ alkylene-(C₃₋₈ cycloalkylene)-S—, —(C₃₋₈ cycloalkylene)-C₁₋₁₀ alkylene-S—, -4 to 14-membered heterocycloalkylene-S—, —C₁₋₁₀ alkylene-(4 to 14-membered heterocycloalkylene)-S—, or -(4 to 14-membered heterocycloalkylene)-C₁-C₁₀ alkylene-S—;

each Z₅ independently is absent, R₅?-R₁₇ or a polyether unit:

each R₅₇ independently is a bond, NR₂₃, S or O;

each R₂₃ independently is hydrogen. C₁₋₆ alkyl, C₆₋₁₀ aryl, C₃₋₈ cycloalkyl, —COOH, or —COO—C₁₋₆ alkyl; and

each Z₆ independently is absent, —C₁₋₁₀ alkyl-R₃—, —C₁₋₁₀ alkyl-NR₅—, —C₁₋₁₀ alkyl-C(O)—, —C₁₋₁₀ alkyl-O—, —C₁₋₁₀ alkyl-S— or —(C₁₋₁₀ alkyl-R₃)_{g1}—C₁₋₁₀ alkyl-C(O)—;

each R₃ independently is —C(O)—NR₅— or —NR₅—C(O)—;

each R₅ independently is hydrogen, C₁₋₆ alkyl, C₆₋₁₀ aryl, C₃₋₈ cycloalkyl, COOH, or COO—C₁₋₆ alkyl; and

g₁ is an integer from 1 to 4.

In some embodiments, Z₄ is

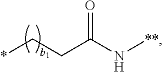

e.g., wherein b₁ is 0, 1 or 4.

In some embodiments, Z₄ is

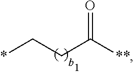

e.g., wherein b₁ is 1 or 4.

In some embodiments, Z₄ is

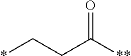

e.g., wherein b₁ is 1.

In some embodiments, Z₄ is

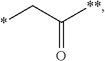

e.g., wherein b₁ is 0.

In some embodiments, each Z₅ independently is a polyalkylene glycol (PAO), including but are not limited to, polymers of lower alkylene oxides, in particular polymers of ethylene oxide, such as, for example, propylene oxide, polypropylene glycols, polyethylene glycol (PEG), polyoxyethylenated polyols, copolymers thereof and block copolymers thereof. In some embodiments, the polyalkylene glycol is a polyethylene glycol (PEG) including, but not limited to, polydisperse PEG, monodisperse PEG and discrete PEG. Polydisperse PEGs are a heterogeneous mixture of sizes and molecular weights whereas monodisperse PEGs are typically purified from heterogeneous mixtures and are therefore provide a single chain length and molecular weight. In some embodiments, the PEG units are discrete PEGs provide a single molecule with defined and specified chain length. In some embodiments, the polyethylene glycol is mPEG.

As used herein a subunit when referring to the PEG unit refers to a polyethylene glycol subunit having the formula

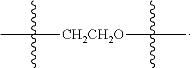

In some embodiments, the PEG unit comprises multiple PEG subunits.

In some embodiments, when z is 2 or 3, at least one Z₅ is a polyalkylene glycol (PAO), e.g., a PEG unit.

In some embodiments, the PEG unit comprises 1 to 6 subunits.

In some embodiments, the PEG unit comprises 1 to 4 subunits.

In some embodiments, the PEG unit comprises 1 to 3 subunits.

In some embodiments, the PEG unit comprises 2 subunits.

In some embodiments, the PEG unit comprises 1 subunit.

In some embodiments, the PEG unit comprises one or multiple PEG subunits linked together by a PEG linking unit. The PEG linking unit that connects one or more chains of repeating CH₂CH₂O— subunits can be Z₆. In some embodiments, Z₆ is —C₁₋₁₀ alkyl-R₃—, —C₂₋₁₀ alkyl-NH—, —C₂₋₁₀ alkyl-C(O)—, —C₂₋₁₀ alkyl-O— or —C₁₋₁₀ alkyl-S, wherein R₃ is —C(O)—NR₅— or —NR₅—C(O)—.

In some embodiments, the PEG linking unit is —C₁₋₁₀ alkyl-C(O)—NH— or —C₁₋₁₀ alkyl-NH—C(O)—. In one embodiment, the PEG linking unit is —(CH₂)₂—C(O)—NH—.

In some embodiments, each Z₅ is absent.

In some embodiments, when z is 2 or 3, at least one Z₅ is absent.

In some embodiments, each Z₅ is —(CH₂—CH₂—O—)₂-.

In some embodiments, when z is 2 or 3, at least one Z₅ is —(CH₂—CH₂—O—)₂—.

In some embodiments, each Z₅ independently is R₇—R17. In some embodiments, each Z₅ independently is R₁₇, NHR₁₇, OR₁₇, or SR₁₇.

In some embodiments, when z is 2 or 3, at least one Z₅ is R₅₇—R₁₇, e.g., R₁₇, NHR₁₇, OR₁₇, or SR₁₇.

In some embodiments, each Z₅ is absent.

In some embodiments, when z is 2 or 3, at least one Z₆ is absent.

In some embodiments, at least one of Z₅ and Z₆ is not absent.

In some embodiments, each Z₆ independently is —C₁₋₁₀ alkyl-R₃—, —C₁₋₁₀ alkyl-NH—, —C₁₋₁₀ alkyl-C(O)—, —C₁₋₁₀ alkyl-O—, —C₁₋₁₀ alkyl-S— or —(C₁₋₁₀ alkyl-R₃)_{g1}—C₁₋₁₀ alkyl-C(O)—. In some embodiments, g₁ is an integer from 1 to 4.

In some embodiments, when z is 2 or 3, at least one Z₆ is —C₁₋₁₀ alkyl-R₃—, —C₁₋₁₀ alkyl-NH—, —C₁₋₁₀ alkyl-C(O)—, —C₁₋₁₀ alkyl-O—, —C₁₋₁₀ alkyl-S— or —(C₁₋₁₀ alkyl-R₃)_{g1}—C₁₋₁₀ alkyl-C(O)—. In some embodiments, g₁ is an integer from 1 to 4.

In some embodiments, each Z₆ independently or at least one Z₆ is —C₂₋₁₀ alkyl-C(O)—, e.g., —(CH₂)₂—C(O)—.

In some embodiments, each Z₆ independently or at least one Z is —C₂₋₁₀ alkyl-R₃)_{g1}—C₂₋₁₀ alkyl-C(O)—, e.g., —(CH₂)₂—C(O)NH—(CH₂)₂—C(O)—.

In some embodiments, each Z₆ independently or at least one Z₆ is —(C₂₋₁₀ alkyl-R₃)_{g1}—C₂₋₁₀ alkyl-C(O)—, e.g., —(CH₂)₂—C(O)NH—(CH₂)₂—NHC(O)—(CH₂)—C(O)—.

In some embodiments, —[(Z₅)—(Z₆)]_{z}— is not absent.

In some embodiments, —[(Z₅)—(Z₆)]_{z}— is a bond.

In some embodiments, —[(Z₅)—(Z₆)]_{z}— is —(CH₂CH₂O)₂—(CH₂)₂—C(O)—NH—(CH₂CH₂O)₂—.

In some embodiments, M^{P}, when present, is

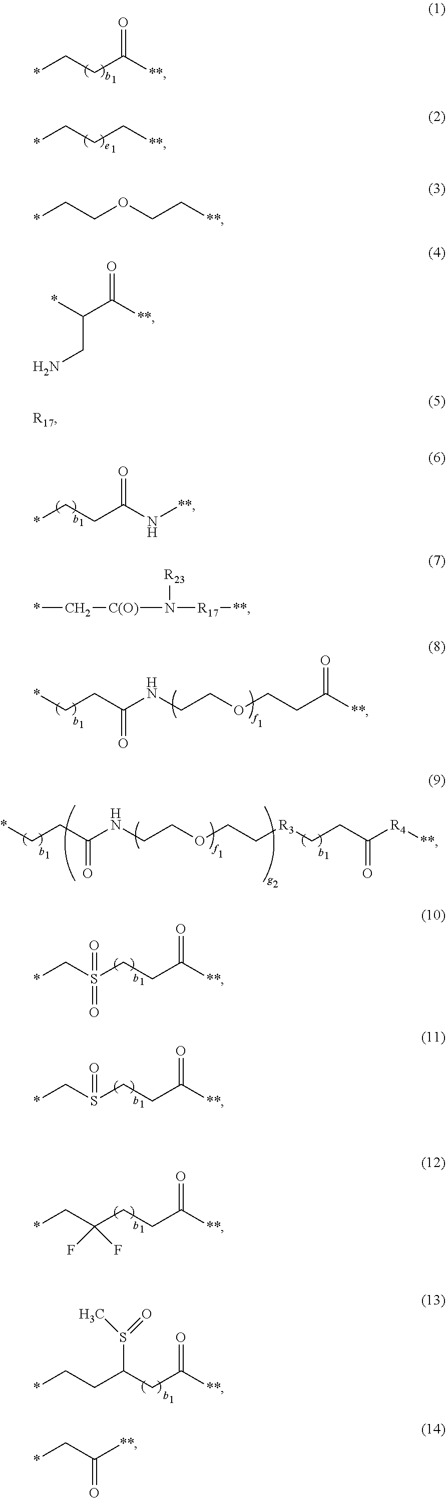

wherein * denotes attachment to L^{P′} or L^{P} and ** denotes attachment to L^{M};

R₃, R₅, R₁₇, and R₂₃ are as defined herein;

R₄ is a bond or —NR₅—(CR₂₀R₂₁)—C(O)—;

each R₂₀ and R₂₁ independently is hydrogen, C₁₋₆ alkyl, C₆₋₁₀ aryl, hydroxylated C₆₋₁₀ aryl, polyhydroxylated C₆₋₁₀ aryl, 5 to 12-membered heterocycle, C₃₋₈ cycloalkyl, hydroxylated C₃₋₈ cycloalkyl, polyhydroxylated C₃₋₈ cycloalkyl or aside chain of a natural or unnatural amino acid;

each b₁ independently is an integer from 0 to 6;

e₁ is an integer from 0 to 8,

each f₁ independently is an integer from 1 to 6; and

g₂ is an integer from 1 to 4.

In some embodiments, b₁ is 1.

In some embodiments, b₁ is 0

In some embodiments, each f₁ independently is 1 or 2.

In some embodiments, f₁ is 2.

In some embodiments, g₂ is 1 or 2.

In some embodiments, g₂ is 2.

In some embodiments, R₁₇ is unsubstituted.

In some embodiments, R₁₇ is optionally substituted.

In some embodiments, R₁₇, is optionally substituted by a basic unit, e.g., —(CH₂)ₓNH₂, —(CH₂)ₓNHR^{a}, and —(CH₂)ₓN(R^{a})₂, wherein x is an integer from 1 to 4 and each R^{a} is independently selected from C₁₋₆ alkyl and C₁₋₆ haloalkyl, or two R^{a} groups are combined with the nitrogen to which they are attached to form an azetidinyl, pyrrolidinyl or piperidinyl group.

In some embodiments, R₁₇ is —C₂₋₅ alkylene-C(═O)— wherein the alkylene is optionally substituted by a basic unit, e.g., —(CH₂)ₓNH₂, —(CH₂)ₓNHR^{a}, and —(CH₂)ₓN(R^{a})₂, wherein x and Rₐ are as defined herein.

In some embodiments, wherein M^{P}, when present, is:

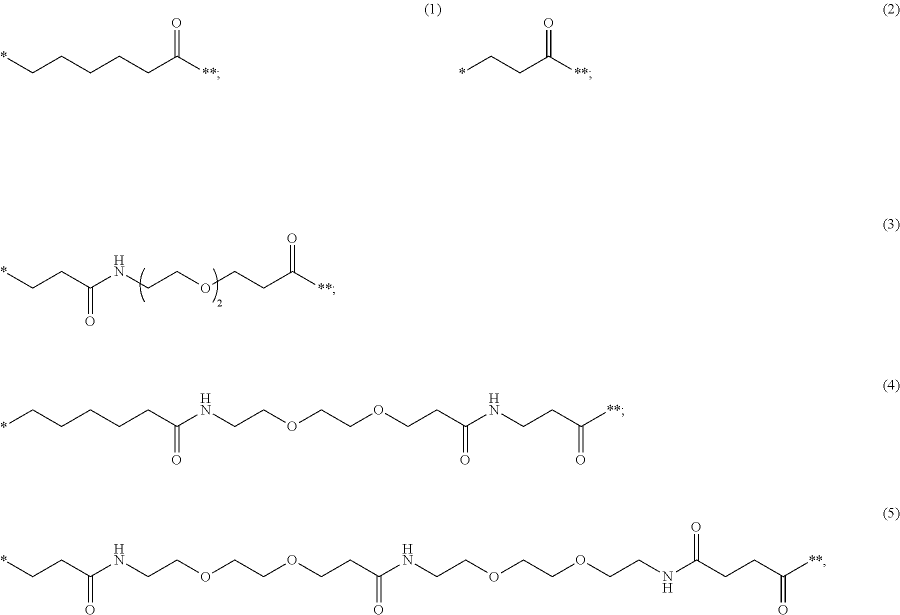

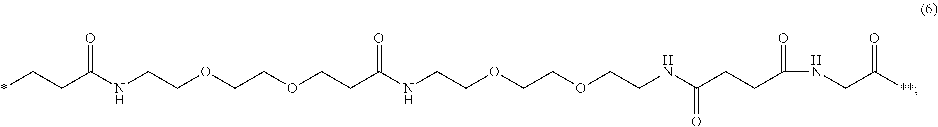

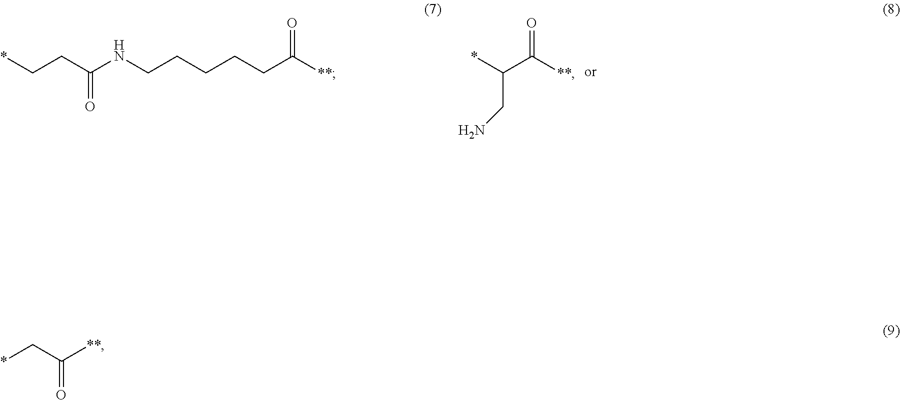

wherein * denotes attachment to L^{P′} or L^{P} and ** denotes attachment to M^{A}.

In some embodiments, wherein M^{P}, when present, is:

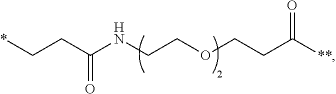

wherein * denotes attachment to L^{P′} or L^{P} and ** denotes attachment to M^{A}.

In some embodiments, wherein M^{P}, when present, is:

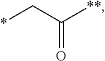

wherein * denotes attachment to L^{P′} or L^{P} and ** denotes attachment to M^{A}.

### M^{A}

In some embodiments, M^{A} is a linker moiety that is capable of connecting one or more drugs and one or more hydrophilic groups to L^{P} or L^{P′}. In some embodiments, M^{A} comprises a peptide moiety of at least two amino acid (AA) units.

The peptide moiety is a moiety that is capable of forming a covalent bond with a -L^{D}-D unit and allows for the attachment of multiple drugs. In some embodiments, peptide moiety comprises a single AA unit or has two or more AA units (e.g., 2 to 10, preferably from 2 to 6, e.g., 2, 3, 4, 5 or 6) wherein the AA units are each independently a natural or non-natural amino acid, an amino alcohol, an amino aldehyde, a diamine, or a polyamine or combinations thereof. If necessary in order to have the requisite number of attachments, at least one of AA units will have a functionalized side chain to provide for attachment of the -L^{D}-D unit. Exemplary functionalized AA units (e.g., amino acids, amino alcohols, or amino aldehydes) include, for example, azido or alkyne functionalized AA units (e.g., amino acid, amino alcohol, or amino aldehyde modified to have an azide group or alkyne group for attachment using click chemistry).

In some embodiments, the peptide moiety has 2 to 12 AA units.

In some embodiments, the peptide moiety has 2 to 10 AA units.

In some embodiments, the peptide moiety has 2 to 6 AA units.

In some embodiments, the peptide moiety has 2, 3, 4, 5 or 6 AA units.

In some embodiments, an AA unit has three attachment sites, (e.g., for attachment to L^{M}, the hydrophilic group (T′) or another AA unit, and to the -L^{D}-D unit). In some embodiments, the AA unit has the formula:

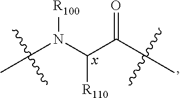

wherein the wavy line indicates attachment sites within the conjugate (e.g., the antibody-drug conjugate (ADC)) of the disclosure or intermediates thereof; and R₁₀₀ and R₁₁₀ are as defined herein.

In some embodiments, an AA unit has two attachment sites (i.e., a terminal unit) and one of the attachment sites shown above can replaced, for example, by H, OH, or an unsubstituted C₁₋₃ alkyl group.

In some embodiments, the peptide moiety comprises at least two AA units of the following formula:

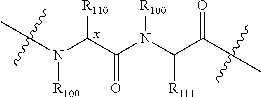

wherein:

each R₁₁₁ independently is H, p-hydroxybenzyl, methyl, isopropyl, isobutyl, sec-butyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl,

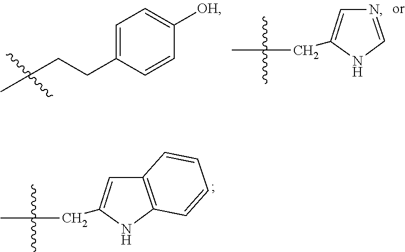 [SMILES: *CCc1ccc(O)cc1 ; *Cc1cnc[nH]1 ; *Cc1cc2ccccc2[nH]1]

the wavy line indicates the attachment sites within the conjugate or intermediates thereof; and

R₁₀₀ and R₁₁₀ are as defined herein.

In some embodiments, the peptide moiety comprises at least two AA units, e.g., cysteine-alanine is:

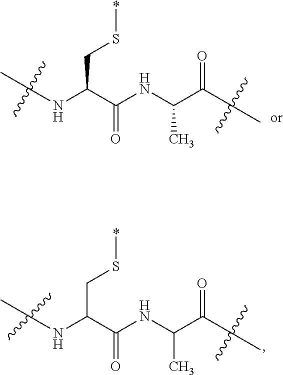 [SMILES: *N[C@@H](CS*)C(=O)N[C@@H](C)C(*)=O ; *NC(CS*)C(=O)NC(C)C(*)=O]

wherein the wavy lines and asterisk indicates attachment sites within the conjugate or intermediates thereof. For example, asterisk indicates attachment site of -L^{D}-D unit or a hydrophilic group. For example, the wavy line next to the carbonyl group indicates attachment site of -L^{D}-D unit or a hydrophilic group. For example, the wavy line next to the amine group indicates attachment site of -L^{D}-D unit or a hydrophilic group. For example, one or two of the wavy lines and asterisk indicate attachment site(s) of one or more -L^{D}-D units or one or more hydrophilic groups.

In some embodiments, the peptide moiety comprises at least two AA units, which provide two attachment sites, e.g., cysteine-alanine is:

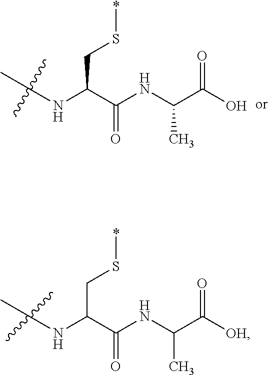 [SMILES: *N[C@@H](CS*)C(=O)N[C@@H](C)C(=O)O ; *NC(CS*)C(=O)NC(C)C(=O)O]

wherein the wavy line and asterisk indicates attachment sites within the conjugate or intermediates thereof. In some embodiments, asterisk indicates attachment site of -L^{D}-D unit or a hydrophilic group. In some embodiments, the wavy line indicates attachment site of -L^{D}-D unit or a hydrophilic group.

One or more AA units (e.g., an amino acid, amino alcohol, amino aldehyde or polyamine) of the peptide moiety can be replaced by an optionally substituted C₁₋₂₀ heteroalkylene (e.g., optionally substituted C₁₋₁₂ heteroalkylene), optionally substituted C₃₋₈ heterocyclo, optionally substituted C₆₋₁₄ arylene, or optionally substituted C₃₋₈ carbocyclo as described herein. The optionally substituted heteroalkylene, heterocycle, arylene or carbocyclo may have one or more functional groups for attachment within a conjugate or intermediates thereof. Suitable substituents include, but are not limited to (═O), —R^{1C}, —R^{1B}, —OR^{1B}, —SR^{1B}, —N(R^{1B})₂, —N(R^{1B})₃, ═NR^{1B}, C(R^{1C})₃, CN, OCN, SCN, N═C═O, NCS, NO, NO₂, ═N₂, N₃, NR^{1B}C(═O)R^{1B}, —C(═O)R^{1B}, —C(═O)N(R^{1B})₂, SO₃⁻, SO₃H, S(═O)₂R^{1B}, —OS(═O)₂OR^{1B}, —S(═O)₂NR^{1B}, —S(═O)R^{1B}, —OP(═O)(OR^{1B}), —P(═O)(OR^{1B})₂, PO₃⁻, PO₃H₂, AsO₂H₂, C(═O)R^{B1}, C(═O)R^{1C}, C(═S)R^{1B}, CO₂R^{1B}, CO₂—, C(═S)OR^{1B}, C(═O)SR^{1B}, C(═S)SR^{1B}, C(═O)N(R^{1B})₂, C(═S)N(R^{B1})₂, and C(═NR^{B1})N(R^{B1})₂, wherein each R^{1C} is independently a halogen (e.g., —F, —CI, —Br, or —I), and each R^{1B} is independently —H, —C₁₋₂₀ alkyl, —C₆₋₂₀ aryl, —C₃₋₁₄ heterocycle, a protecting group or a prodrug moiety.

In some embodiments, the one or more substituents for the heteroalkylene, heterocycle, arylene or carbocyclo are selected from (═O), R^{1C}, R^{1B}, OR^{1B}, SR^{1B}, and N(R^{1B})₂.

In some embodiments, the peptide moiety can be a straight chain or branched moiety of having the Formula:

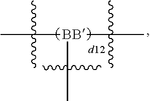

wherein:

each BB′ is independently an amino acid, optionally substituted C₁₋₂₀ heteroalkylene (e.g., optionally substituted C₁₋₁₂ heteroalkylene), optionally substituted C₃₋₈ heterocyclo, optionally substituted C₆₋₁₄ arylene, or optionally substituted C₃-C₈ carbocyclo;

d₁₂ is an integer from 1 to 10; and

the wavy line indicates the covalent attachment sites within the conjugate or intermediate thereof.

In some embodiments, d₁₂ is an integer from 2 to 10.

In some embodiments, d₁₂ is an integer from 2 to 6.

In some embodiments, d₁₂ is an integer from 4, 5 or 6.

In some embodiments, d₁₂ is an integer from 5 or 6.

In some embodiments, the optionally substituted heteroalkylene, heterocycle, arylene or carbocyclo have functional groups for attachments between the BB′ subunits and/or for attachments within a conjugate or intermediates thereof disclosed herein.

In some embodiments, the peptide moiety comprises no more than 2 optionally substituted C₁₋₂₀ heteroalkylenes, optionally substituted C₃₋₁₈ is heterocyclos, optionally substituted C₆₋₁₄ arylenes, or optionally substituted C₃₋₈ carbocyclos.

In other embodiments, the peptide moiety comprises no more than 1 optionally substituted C₁₋₂₀ heteroalkylenes, optionally substituted C₃₋₁₈ heterocyclos, optionally substituted C₆₋₁₄ arylenes, or optionally substituted C₃₋₈ carbocyclos. The optionally substituted heteroalkylene, heterocycle, arylene or carbocyclo will have functional groups for attachment between the BB′ subunits and/or for attachments within a conjugate or intermediates thereof disclosed herein.

In some embodiments, at least one BB′ is an amino acid. In some embodiments, the amino acid can be an alpha, beta, or gamma amino acid, which can be natural or non-natural. The amino acid can be a D or L isomer.

In some embodiments, attachment within the peptide moiety or with the other components of the conjugate (or intermediate thereof, or scaffold) can be, for example, via amino, carboxy, or other functionalities.

In some embodiments, each amino acid of the peptide moiety can be independently D or L isomer of a thiol containing amino acid. The thiol containing amino acid can be, for example, cysteine, homocysteine, or penicillamine.

In some embodiments, each amino acid that comprises the peptide moiety can be independently the L- or D-isomers of the following amino acids: alanine (including β-alanine), arginine, aspartic acid, asparagine, cysteine, histidine, glycine, glutamic acid, glutamine, phenylalanine, lysine, leucine, methionine, serine, tyrosine, threonine, tryptophan, proline, ornithine, penicillamine, aminoalkynoic acid, aminoalkanedioic acid, heterocyclo-carboxylic acid, citrulline, statine, diaminoalkanoic acid, stereoisomers thereof (e.g., isoaspartic acid and isoglutamic acid), and derivatives thereof.

In some embodiments, each amino acid that comprises the peptide moiety is independently cysteine, homocysteine, penicillamine, ornithine, lysine, serine, threonine, glycine, glutamine, alanine, aspartic acid, glutamic acid, selenocysteine, proline, glycine, isoleucine, leucine, methionine, valine, alanine, or a stereoisomers thereof (e.g., isoaspartic acid and isoglutamic acid).

In some embodiments, the peptide moiety comprises a monopeptide, a dipeptide, tripeptide, tetrapeptide, or pentapeptide.

In some embodiments, the peptide moiety contains at least about five amino acids (e.g., 5, 6, 7, 8, 9, or 10 amino acids).

In some embodiments, the peptide moiety contains at most about ten amino acids.

In some embodiments, the peptide moiety comprises a pentapeptide.

In some embodiments, each amino acid that comprises the peptide moiety is independently glycine, serine, glutamic acid, lysine, aspartic acid and cysteine.

In some embodiments, the peptide moiety comprises at least four glycines and at least one serine, e.g., (glycine)₄ and serine wherein the serine is at any position along the peptide chain, such as, for example, (serine)-(glycine)₄; (glycine)-(serine)-(glycine)₃; (glycine)₂-(serine)-(glycine)₂; (glycine)₃-(serine)-(glycine); or (glycine)₄-(serine).

In some embodiments, the peptide moiety comprises (glycine)₄-(serine) or (serine)-(glycine)₄.

In some embodiments, the peptide moiety comprises at least four glycines and at least one glutamic acid e.g., (glycine)₄ and glutamic acid wherein the glutamic acid is at any position along the peptide chain, such as, for example, (glutamic acid)-(glycine)₄; (glycine)-(glutamic acid)-(glycine)₃; (glycine)₂-(glutamic acid)-(glycine)₂; (glycine)₃-(glutamic acid)-(glycine); or (glycine)₄-(glutamic acid).

In some embodiments, the peptide moiety comprises (glutamic acid)-(glycine)₄; or (glycine)₄-(glutamic acid).

In some embodiments, the peptide moiety comprises (β-alanine)-(glycine)-(serine) wherein the serine is at any position along the peptide chain, such as, for example, (β-alanine)-(serine)-(glycine)₄; (s-alanine)-(glycine)-(serine)-(glycine)₃; (β-alanine)-(glycine)₂-(serine)-(glycine)₂; (β-alanine)-(glycine)₃-(serine)-(glycine); or (β-alanine)-(glycine)-(serine).

In some embodiments, the peptide moiety comprises (glycine)₄-(serine)-(glutamic acid) wherein the serine is at any position along the peptide chain, such as, for example, (serine)-(glycine)₄-(glutamic acid); (glycine)-(serine)-(glycine)₃-(glutamic acid); (glycine)₂-(serine)-(glycine)₂-(glutamic acid); (glycine)₃-(serine)-(glycine)-(glutamic acid); or (glycine)₄-(serine)-(glutamic acid). In another embodiment, the peptide moiety comprises (β-alanine)-(glycine)₄-(serine)-(glutamic acid) wherein the serine is at any position along the peptide chain, such as, for example, (β-alanine)-(serine)-(glycine)₄-(glutamic acid); (β-alanine)-(glycine)-(serine)-(glycine)₃-(glutamic acid); (β-alanine)-(glycine)₂-(serine)-(glycine)₂-(glutamic acid); (β-alanine)-(glycine)₃-(serine)-(glycine)-(glutamic acid); or (β-alanine)-(glycine)₄-(serine)-(glutamic acid).

In some embodiments, when at least one of hydrophilic groups (T′) is a polyalcohol or derivative thereof (e.g., an amino polyalcohol) or a glucosyl-amine or a di-glucosyl-amine or a tri-glucosyl-amine, M^{A} does not have to comprise a peptide moiety. In some embodiments, M^{A} comprises one or more of the following:

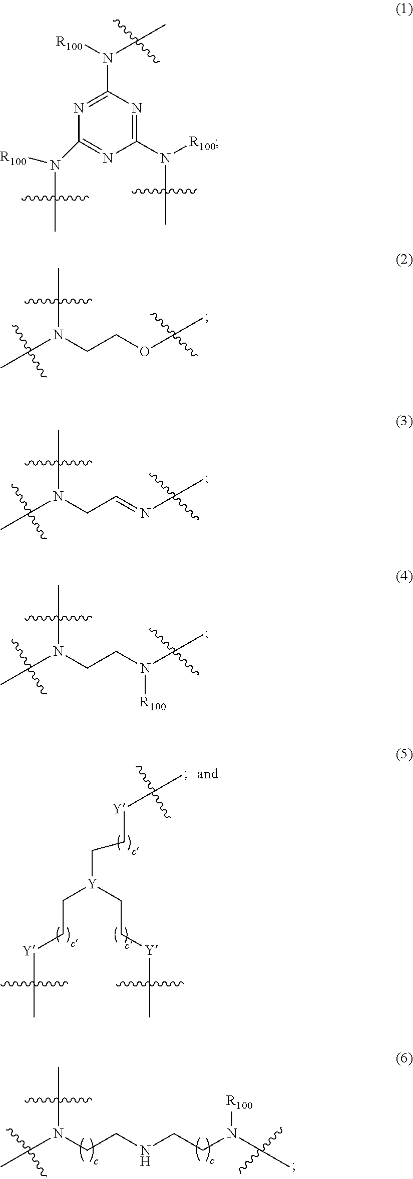

wherein

the wavy line indicates attachment sites within the conjugate (e.g., the antibody-drug conjugate (ADQ)) of the disclosure or intermediates thereof; and R₁₀₀ and R₁₁₀ are as defined herein.

In some embodiments, R₁₁₀ is:

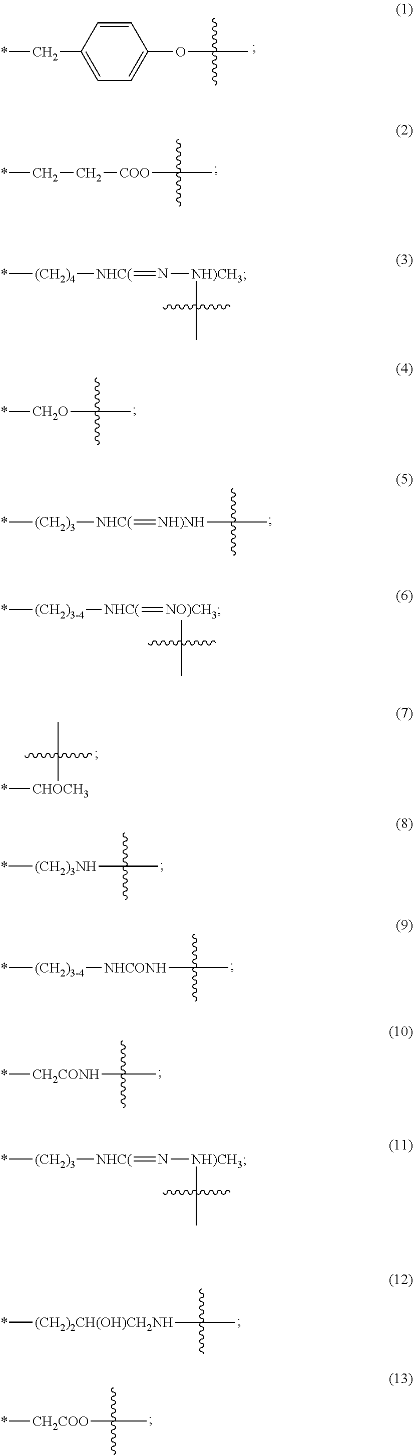

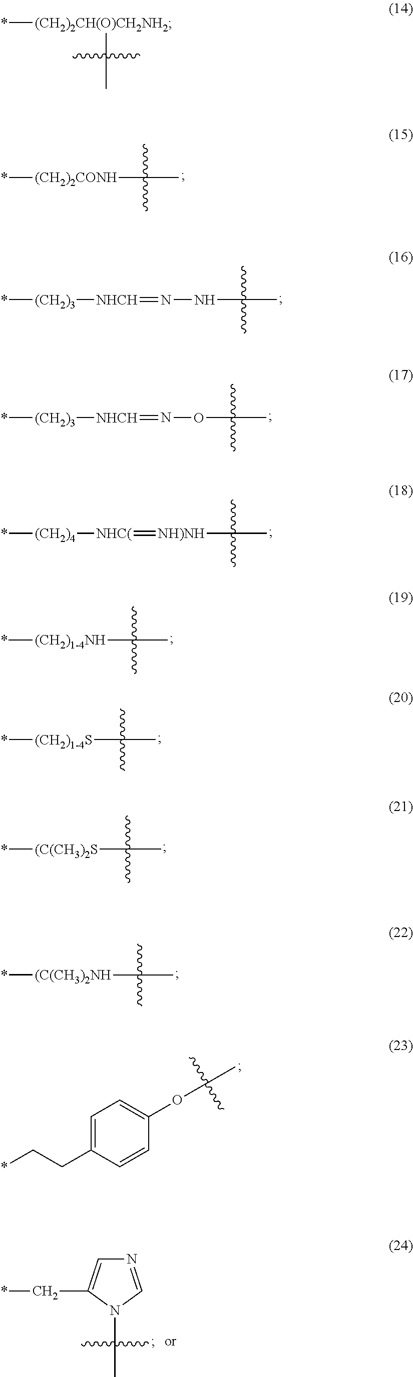

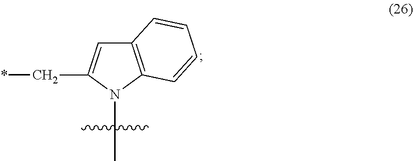 [SMILES: *Cc1cc2ccccc2n1*]

wherein the asterisk indicates attachment to the carbon labeled x and the wavy line indicates one of the three attachment sites.

In some embodiments, R₁₀₀ is independently selected from hydrogen and CH₃.

In some embodiments, Y is N.

In some embodiments, Y is CH.

In some embodiments, R₁₀₀ is H or CH₃.

In some embodiments, each c′ is independently an integer from 1 to 3.

In some embodiments, R₁₁₀ is not

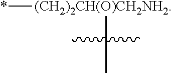

L^{D} and W^{D}

In some embodiments, each occurrence of L^{D} is independently a divalent linker moiety connecting D to M^{A} and comprises at least one cleavable bond such that when the bond is broken, D is released in an active form for its intended therapeutic effect.

In some embodiments, L^{D} is a component of the Releasable Assembly Unit. In other embodiments, L^{D} is the Releasable Assembly Unit.

In some embodiments, L^{D} comprises one cleavable bond.

In some embodiments, L^{D} comprises multiple cleavage sites or bonds.

Functional groups for forming a cleavable bond can include, for example, sulfhydryl groups to form disulfide bonds, aldehyde, ketone, or hydrazine groups to form hydrazone bonds, hydroxylamine groups to form oxime bonds, carboxylic or amino groups to form peptide bonds, carboxylic or hydroxy groups to form ester bonds, and sugars to form glycosidic bonds. In some embodiments, L^{D} comprises a disulfide bond that is cleavable through disulfide exchange, an acid-labile bond that is cleavable at acidic pH, and/or bonds that are cleavable by hydrolases (e.g., peptidases, esterases, and glucuronidases). In some embodiments, L^{D} comprises a carbamate bond (i.e., —O—C(O)—NR—, in which R is H or alkyl or the like).

The structure and sequence of the cleavable bond(s) in L^{D} can be such that the bond(s) is cleaved by the action of enzymes present at the target site. In other embodiments, the cleavable bond(s) can be cleavable by other mechanisms.

In some embodiments, the cleavable bond(s) can be enzymatically cleaved by one or more enzymes, including a tumor-associated protease, to liberate the Drug moiety or D, which in one embodiment is protonated in vivo upon release to provide a Drug moiety or D.

In certain embodiments, L^{D} can comprise one or more amino acids. In some embodiments, each amino acid in L^{D} can be natural or unnatural and/or a D- or L-isomer provided that there is a cleavable bond. In some embodiments, L^{D} comprising an alpha, beta, or gamma amino acid that can be natural or non-natural. In some embodiments, L^{D} comprises 1 to 12 (e.g., 1 to 6, or 1 to 4, or 1 to 3, or 1, 2, 3, 4, 5, 6, 7, 8, 9, 10, 11, or 12) amino acids in contiguous sequence. In certain embodiments, L^{D} can comprise only natural amino acids. In other embodiments, L^{D} can comprise only non-natural amino acids. In some embodiments, L^{D} can comprise a natural amino acid linked to a non-natural amino acid. In some embodiments, L^{D} can comprise a natural amino acid linked to a D-isomer of a natural amino acid. An exemplary L^{D} comprises a dipeptide such as -Val-Cit-, -Phe-Lys-, -Ala-Ala- or -Val-Ala-.

In some embodiments, L^{D} comprises, a monopeptide, a dipeptide, a tripeptide, a tetrapeptide, a pentapeptide, a hexapeptide, a heptapeptide, an octapeptide, a nonapeptide, a decapeptide, an undecapeptide or a dodecapeptide unit.

In some embodiments, L^{D} comprises a peptide (e.g., of 1 to 12 amino acids), which is conjugated directly to the drug moiety. In some such embodiments, the peptide is a single amino acid or a dipeptide.

In some embodiments, each amino acid in L^{D} is independently selected from alanine, -alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, selenocysteine, ornithine, penicillamine, aminoalkanoic acid, aminoalkynoic acid, aminoalkanedioic acid, aminobenzoic acid, amino-heterocyclo-alkanoic acid, heterocyclo-carboxylic acid, citrulline, statine, diaminoalkanoic acid, and derivatives thereof.

In some embodiments, each amino acid is independently selected from alanine, β-alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, citrulline and selenocysteine.

In some embodiments, each amino acid is independently selected from the group consisting of alanine, β-alanine, arginine, aspartic acid, asparagine, histidine, glycine, glutamic acid, glutamine, phenylalanine, lysine, leucine, serine, tyrosine, threonine, isoleucine, proline, tryptophan, valine, citrulline and derivatives thereof.

In some embodiments, each amino acid is selected from the proteinogenic or the non-proteinogenic amino acids.

In some embodiments, each amino acid in L^{D} can be independently selected from L- or D-isomers of the following amino acids: alanine, β-alanine, arginine, aspartic acid, asparagine, cysteine, histidine, glycine, glutamic acid, glutamine, phenylalanine, lysine, leucine, methionine, serine, tyrosine, threonine, tryptophan, proline, ornithine, penicillamine, aminoalkynoic acid, aminoalkanedioic acid, heterocyclo-carboxylic acid, citrulline, statine, diaminoalkanoic acid, valine, citrulline or derivatives thereof.

In some embodiments, each amino acid in L^{D} is independently cysteine, homocysteine, penicillamine, ornithine, lysine, serine, threonine, glycine, glutamine, alanine, aspartic acid, glutamic acid, selenocysteine, proline, glycine, isoleucine, leucine, methionine, valine, citrulline or alanine.

In some embodiments, each amino acid in L^{D} is independently selected from L-isomers of the following amino acids: alanine, β-alanine, arginine, aspartic acid, asparagine, histidine, glycine, glutamic acid, glutamine, phenylalanine, lysine, leucine, serine, tyrosine, threonine, isoleucine, tryptophan, citrulline or valine.

In some embodiments, each amino acid in L^{D} is independently selected from D-isomers of the following amino acids: alanine, β-alanine, arginine, aspartic acid, asparagine, histidine, glycine, glutamic acid, glutamine, phenylalanine, lysine, leucine, serine, tyrosine, threonine, isoleucine, tryptophan, citrulline or valine.

In some embodiments, each amino acid in L^{D} is alanine, 3-alanine, glycine, glutamic acid, isoglutamic acid, isoaspartic acid, valine, citrulline or aspartic acid.

In one embodiment, L^{D} comprises β-alanine.

In another embodiment, L^{D} comprises (β-alanine)-(alanine).

In another embodiment, L^{D} comprises (β-alanine)-(glutamic acid).

In another embodiment, L^{D} comprises (β-alanine)-(isoglutamic acid).

In another embodiment, L^{D} comprises (β-alanine)-(aspartic acid).

In another embodiment, L^{D} comprises (β-alanine)-(isoaspartic acid).

In another embodiment, L^{D} comprises (β-alanine)-(valine).

In another embodiment, L^{D} comprises (β-alanine)-(valine)-(alanine).

In another embodiment, L^{D} comprises (β-alanine)-(alanine)-(alanine).

In another embodiment, L^{D} comprises (β-alanine)-(valine)-(citruline).

In another embodiment, L^{D} comprises (β-alanine)-(valine)-(lys).

In another embodiment, L^{D} comprises (β-alanine)-(lys).

In another embodiment, L^{D} comprises (β-alanine)-(gly)-(gly)-(gly).

In some embodiments, L^{D} comprises:
(i) (β-alanine)-(alanine)-(alanine); or
(ii) (β-alanine)-(valine)-(alanine).

In some embodiments, L^{D} comprises a carbamate bond in addition to one or more amino acids.

In some embodiments, L^{D} can be designed and optimized in their selectivity for enzymatic cleavage by a particular enzyme, e.g., a tumor-associated protease.

In some embodiments, L^{D} comprises a bond whose cleavage is catalyzed by cathepsin B, C and D, or a plasmin protease.

In some embodiments, L^{D} comprises a sugar cleavage site. In some such embodiments, L^{D} comprises a sugar moiety (Su) linked via an oxygen glycosidic bond to a self-immolative group. A “self-immolative group” can be a tri-functional chemical moiety that is capable of covalently linking together three spaced chemical moieties (i.e., the sugar moiety (via a glycosidic bond), a drug moiety (directly or indirectly), and M^{A} (directly or indirectly). The glycosidic bond will be one that can be cleaved at the target site to initiate a self-immolative reaction sequence that leads to a release of the drug.

In some embodiments, L^{D} comprises a sugar moiety (Su) linked via a glycoside bond (—O′—) to a self-immolative group (K) of the formula:

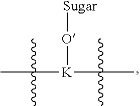

wherein the self-immolative group (K) forms a covalent bond with the drug moiety (directly or indirectly) and also forms a covalent bond with M^{A} (directly or indirectly). Examples of self-immolative groups are described in, e.g., WO 2015/057699, the contents of which are hereby incorporated by reference in its entirety.

In some embodiments, when not connected to or prior to connecting to the PBD drug moiety, L^{D} comprises a functional a functional group W^{D}. Each W^{D} independently can be a functional group as listed for W^{P}. In some embodiments, each W independently is

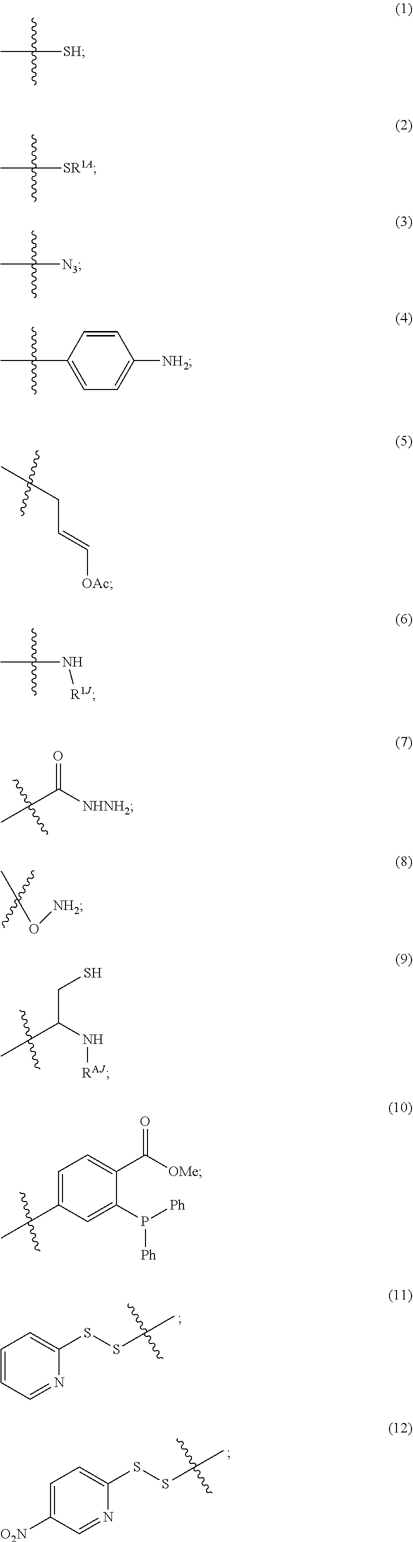

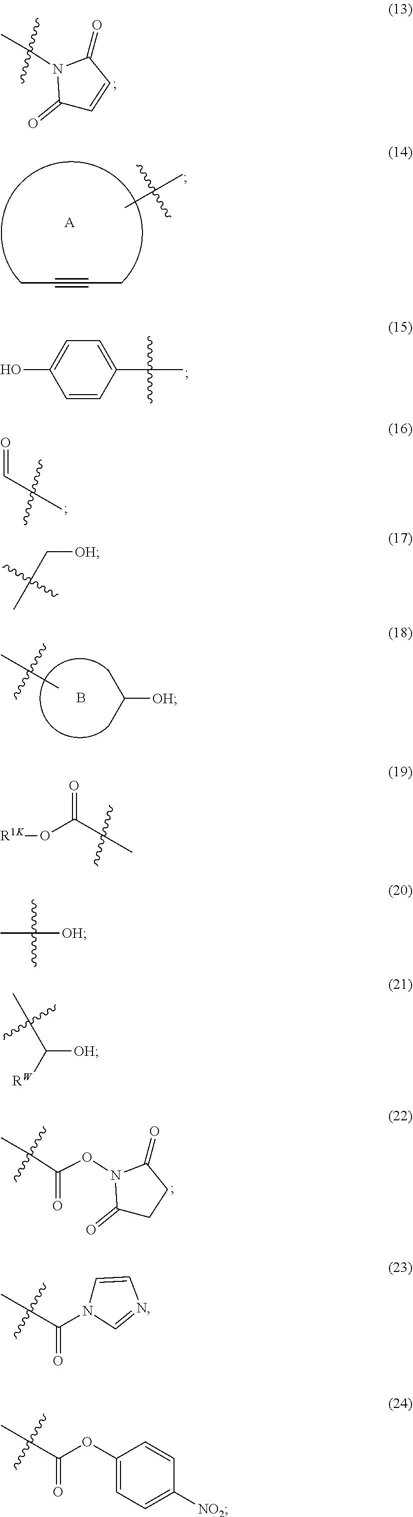

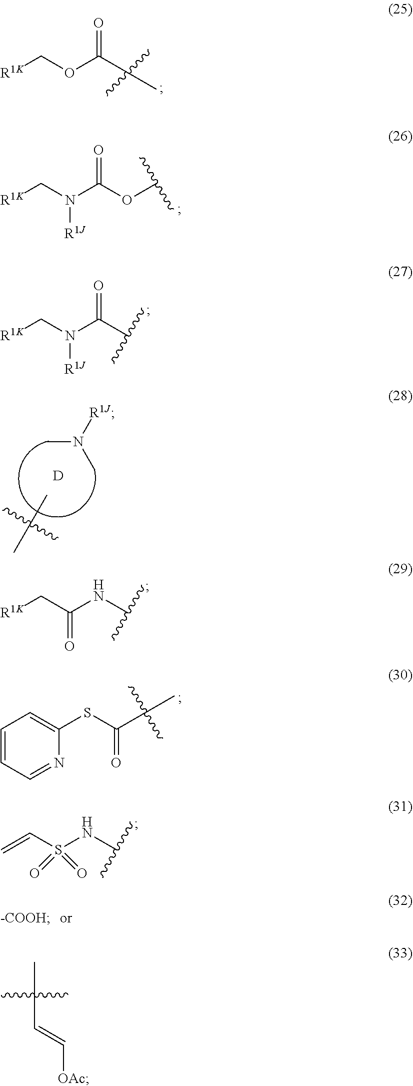

in which R^{1A} is a sulfur protecting group, each of ring A and B, independently, is cycloalkyl or heterocycloalkyl, R^{W} is an aliphatic, heteroaliphatic, carbocyclic or heterocycloalkyl moiety; ring D is heterocycloalkyl; R^{1J} is hydrogen or an aliphatic, heteroaliphatic, carbocyclic, or heterocycloalkyl moiety; and R^{1K} is a leaving group (e.g., halide or RC(O)O— in which R is hydrogen or an aliphatic, heteroaliphatic, carbocyclic, or heterocycloalkyl moiety).

In some embodiments, W^{D} is

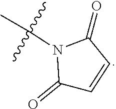 [SMILES: *N1C(=O)C=CC1=O]

In some embodiments, W^{D} is

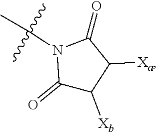

wherein one of Xₐ and X_{b} is H and the other is a maleimido blocking moiety.

In some embodiments, W^{D} is

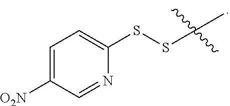 [SMILES: *SSc1ccc([N+](=O)[O-])cn1]

### T′

In some embodiments, the hydrophilic group (T′) included in the conjugates or scaffolds of the disclosure is a water-soluble and substantially non-antigenic polymer. Examples of the hydrophilic group, include, but are not limited to, polyalcohols, polyethers, polyanions, polycations, polyphosphoric acids, polyamines, polysaccharides, polyhydroxy compounds, polylysines, and derivatives thereof. One end of the hydrophilic group (T′) can be functionalized so that it can be covalently attached to the Multifunctional Linker or M^{A} linker (e.g., to an amino acid in the M^{A} linker) by means of a non-cleavable linkage or via a cleavable linkage. Functionalization can be, for example, via an amine, thiol, NHS ester, maleimide, alkyne, azide, carbonyl, or other functional group. The other terminus (or termini) of the hydrophilic group (T′) will be free and untethered. By “untethered”, it is meant that the hydrophilic group (T′) will not be attached to another moiety, such as D or a Drug Moiety, Releasable Assembly Unit, or other components of the conjugates or scaffolds of the disclosure. The free and untethered end of the hydrophilic group (T′) may include a methoxy, carboxylic acid, alcohol or other suitable functional group. The methoxy, carboxylic acid, alcohol or other suitable functional group acts as a cap for the terminus or termini of the hydrophilic group.

A cleavable linkage refers to a linkage that is not substantially sensitive to cleavage while circulating in the plasma but is sensitive to cleavage in an intracellular or intratumoral environment. A non-cleavable linkage is one that is not substantially sensitive to cleavage in any biological environment. Chemical hydrolysis of a hydrazone, reduction of a disulfide, and enzymatic cleavage of a peptide bond or glycosidic linkage are examples of cleavable linkages. Exemplary attachments of the hydrophilic group (T′) are via amide linkages, ether linkages, ester linkages, hydrazone linkages, oxime linkages, disulfide linkages, peptide linkages or triazole linkages. In some embodiments, the attachment of the hydrophilic group (T′) to the Multifunctional Linker or M^{A} linker (e.g., to an amino acid in the M^{A} linker) is via an amide linkage.

For those embodiments wherein the conjugate or scaffold of the disclosure comprises more than one hydrophilic groups, the multiple hydrophilic groups may be the same or different chemical moieties (e.g., hydrophilic groups of different molecular weight, number of subunits, or chemical structure). The multiple hydrophilic groups can be attached to the Multifunctional Linker or M^{A} linker at a single attachment site or different sites.

The addition of the hydrophilic group (T′) may have two potential impacts upon the pharmacokinetics of the resulting conjugate. The desired impact is the decrease in clearance (and consequent in increase in exposure) that arises from the reduction in non-specific interactions induced by the exposed hydrophobic elements of the drug or drug-linker. The second impact is undesired impact and is the decrease in volume and rate of distribution that may arise from the increase in the molecular weight of the conjugate. Increasing the molecular weight of the hydrophilic group (T′) increases the hydrodynamic radius of a conjugate, resulting in decreased diffusivity that may diminish the ability of the conjugate to penetrate into a tumor. Because of these two competing pharmacokinetic effects, it is desirable to use a hydrophilic group (T′) that is sufficiently large to decrease the conjugate clearance thus increasing plasma exposure, but not so large as to greatly diminish its diffusivity, which may reduce the ability of the conjugate to reach the intended target cell population.

In some embodiments, the hydrophilic group, includes, but is not limited to, a sugar alcohol (also known as polyalcohol, polyhydric alcohol, alditol or glycitol, such as inositol, glycerol, erythritol, threitol, arabitol, xylitol, ribitol, galactitol, mannitol, sorbitol, and the like) or a derivative thereof (e.g., amino polyalcohol), carbohydrate (e.g., a saccharide), a polyvinyl alcohol, a carbohydrate-based polymer (e.g., dextrans), a hydroxypropylmethacrylamide (HPMA), a polyalkylene oxide, and/or a copolymer thereof.

In some embodiments, the hydrophilic group (T′) comprises a plurality of hydroxyl (“—OH”) groups, such as moieties that incorporate monosaccharides, oligosaccharides, polysaccharides, and the like. In yet another embodiment the hydrophilic group (T′) comprises a plurality of —(CR₅₈OH)— groups, wherein R₅₈ is hydrogen or C₁₋₈ alkyl.

In some embodiments, the hydrophilic group (T′) comprises one or more of the following fragments of the formula:

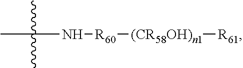

in which

n₁ is an integer from 0 to about 6;

each R₅₈ is independently hydrogen or C₁₋₈ alkyl;

R₆₀ is a bond, a C₁₋₆ alkyl linker, or —CHR₅₉— in which R₅₉ is H, alkyl, cycloalkyl, or arylalkyl;

R₆₁ is CH₂OR₆₂, COOR₆₂, —(CH₂)ₙ₂COOR₆₂, or a heterocycloalkyl substituted with one or more hydroxyl;

R₆₂ is H or C₁₋₈ alkyl; and

n₂ is an integer from 1 to about 5.

In some embodiments, R₅₈ is hydrogen, R₆₀ is a bond or a C₁₋₆ alkyl linker, n₁ is an integer from 1 to about 6, and R₆₁ is CH₂OH or COOH. In some embodiments, R₅₈ is hydrogen, R₆₀ is —CHR₅₉—, n₁ is 0, and R₆₁ is a heterocycloalkyl substituted with one or more hydroxyl, e.g., a monosaccharide.

In some embodiments, the hydrophilic group (T′) comprises a glucosyl-amine, a diamine or a tri-amine.

In some embodiments, the hydrophilic group (T′) comprises one or more of the following fragments or a stereoisomer thereof:

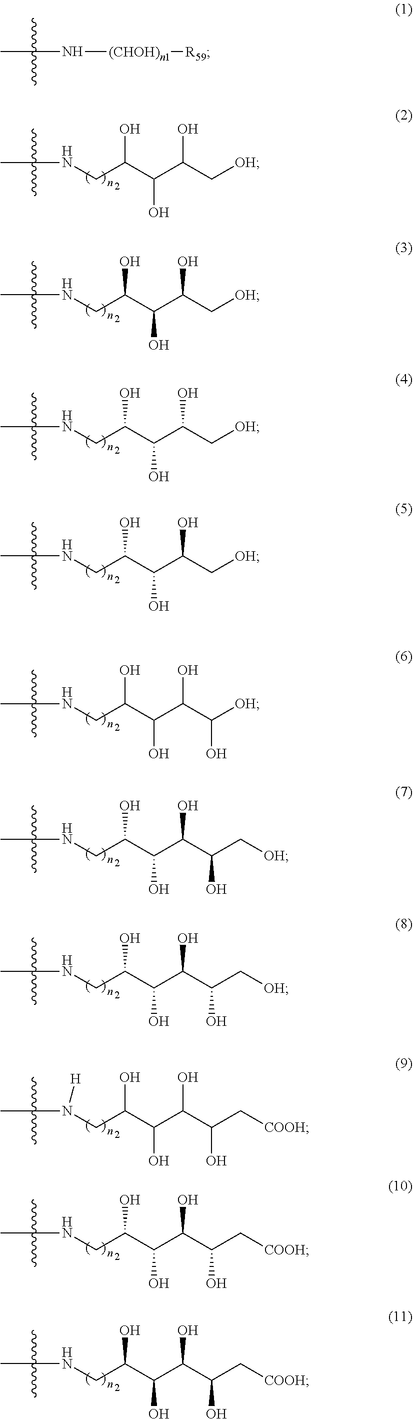

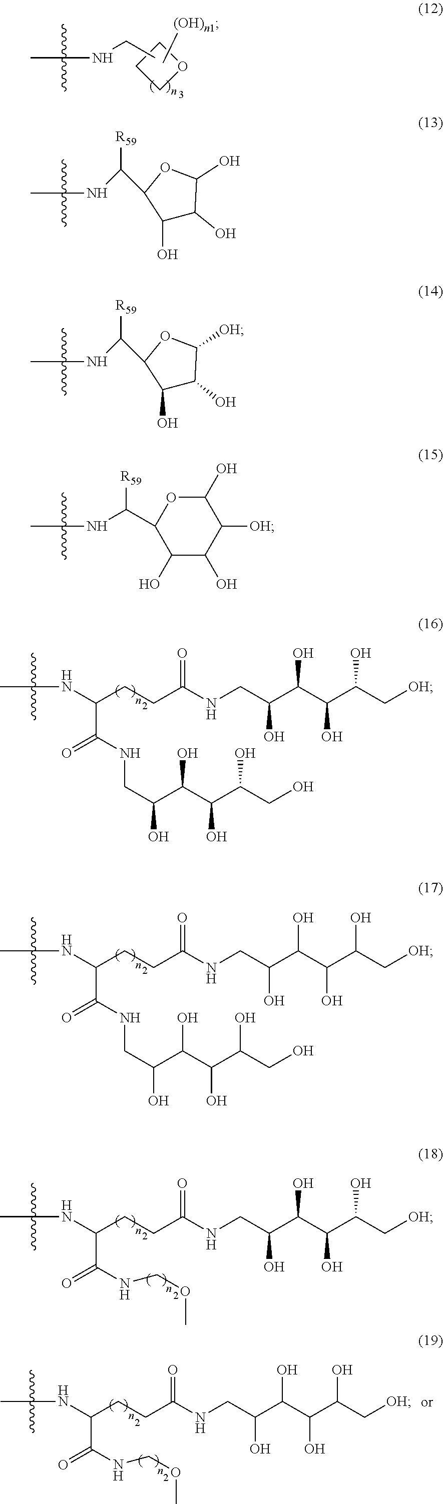

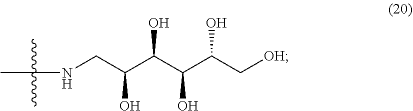

wherein:

R₅₉ is H, alkyl, cycloalkyl, or arylalkyl;

n₁ is an integer from 1 to about 6;

n₂ is an integer from 1 to about 5; and

n₃ is an integer from about 1 to about 3.

It is understood that all stereochemical forms of the hydrophilic groups are contemplated herein. In some embodiments, in the above formula, the hydrophilic group (T′) may be derived from ribose, xylose, glucose, mannose, galactose, or other sugar and retain the stereochemical arrangements of pendant hydroxyl and alkyl groups present on those molecules. In addition, it is to be understood that in the foregoing formulae, various deoxy compounds are also contemplated. Illustratively, one or more of the following features are contemplated for the hydrophilic groups when applicable:

In some embodiments, n₃ is 2 or 3.

In some embodiments, n₁ is 1, 2, or 3.

In some embodiments, n₂ is 1.

In some embodiments, R₅₉ is hydrogen.

In some embodiments, the hydrophilic group (T′) comprises:

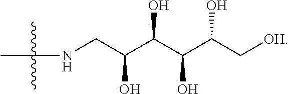 [SMILES: *NC[C@H](O)[C@@H](O)[C@H](O)[C@H](O)CO]

In some embodiments, the hydrophilic group (T′) comprises:

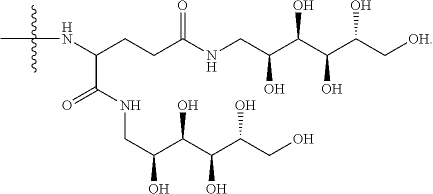 [SMILES: *NC(CCC(=O)NC[C@H](O)[C@@H](O)[C@H](O)[C@H](O)CO)C(=O)NC[C@H](O)[C@@H](O)[C@H](O)[C@H](O)CO]

In some embodiments, the hydrophilic group (T′) comprises:

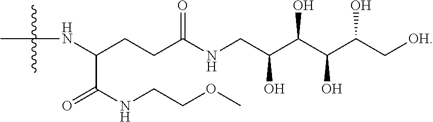 [SMILES: *NC(CCC(=O)NC[C@H](O)[C@@H](O)[C@H](O)[C@H](O)CO)C(=O)NCCOC]

In some embodiments the hydrophilic group (T′) comprises

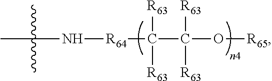

in which

n₄ is an integer from 1 to about 25;

each R₆₃ is independently hydrogen or C₁₋₈ alkyl;

R₆₄ is a bond or a C₁₋₈ alkyl linker;

R₆₅ is H, C₁₋₈ alkyl, —(CH₂)ₙ₂COOR₆₂, or —(CH₂)ₙ₂COR₆₆;

R₆₂ is H or C₁₋₈ alkyl;

R₆₆ is

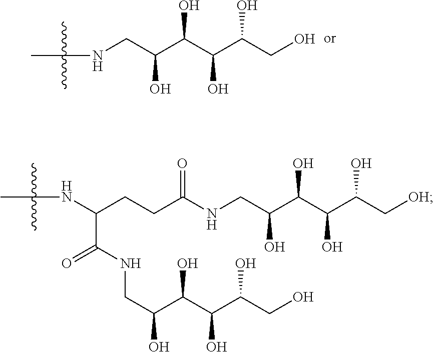 [SMILES: *NC[C@H](O)[C@@H](O)[C@H](O)[C@H](O)CO ; *NC(CCC(=O)NC[C@H](O)[C@@H](O)[C@H](O)[C@H](O)CO)C(=O)NC[C@H](O)[C@@H](O)[C@H](O)[C@H](O)CO]

and

n₂ is an integer from 1 to about 5.

In some embodiments, the hydrophilic group (T′) comprises:

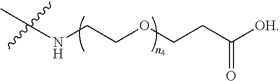

In some embodiments, n₄ is an integer from about 2 to about 20, from about 4 to about 16, from about 6 to about 12, from about 8 to about 12.

In some embodiments, n₄ is 6, 7, 8, 9, 10, 11, or 12.

In some embodiments, n₄ is 8 or 12.

In some embodiments, the hydrophilic group (T′) comprises:

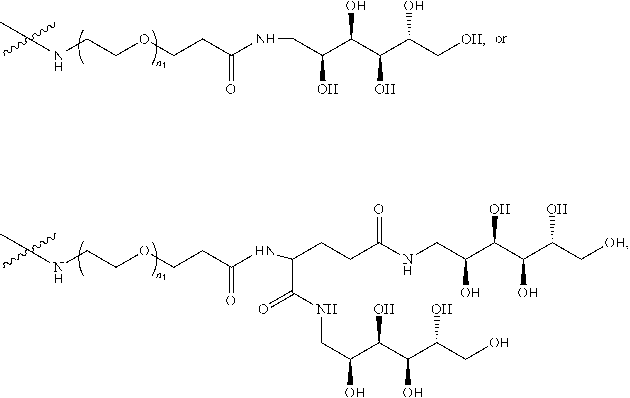

in which n₄ is an integer from about 2 to about 20, from about 4 to about 16, from about 6 to about 12, from about 8 to about 12.

In some embodiments, n₄ is 6, 7, 8, 9, 10, 11, or 12.

In some embodiments, n₄ is 8 or 12.

In some embodiments, the hydrophilic group (T′) comprises a polyether, e.g., a polyalkylene glycol (PAO). PAO includes but is not limited to, polymers of lower alkylene oxides, in particular polymers of ethylene oxide, such as, for example, propylene oxide, polypropylene glycols, polyethylene glycol (PEG), polyoxyethylenated polyols, copolymers thereof and block copolymers thereof. In other embodiments the polyalkylene glycol is a polyethylene glycol (PEG) including, but not limited to, polydisperse PEG, monodisperse PEG and discrete PEG. Polydisperse PEGs are a heterogeneous mixture of sizes and molecular weights whereas monodisperse PEGs are typically purified from heterogeneous mixtures and are therefore provide a single chain length and molecular weight. In another embodiment, the PEG units are discrete PEGs provide a single molecule with defined and specified chain length. In some embodiments, the polyethylene glycol is mPEG.

In some embodiments, the hydrophilic group (T′) comprises a PEG unit which comprises one or multiple polyethylene glycol chains. The polyethylene glycol chains can be linked together, for example, in a linear, branched or star shaped configuration. The PEG unit, in addition to comprising repeating polyethylene glycol subunits, may also contain non-PEG material (e.g., to facilitate coupling of multiple PEG chains to each other or to facilitate coupling to the amino acid). Non-PEG material refers to the atoms in the PEG chain that are not part of the repeating —CH₂CH₂O— subunits. In one embodiment, the PEG chain can comprise two monomeric PEG chains linked to each other via non-PEG elements. In another embodiment, the PEG Unit can comprise two linear PEG chains attached to a central core that is attached to the amino acid (i.e., the PEG unit itself is branched).

The PEG unit may be covalently bound to the Multifunctional Linker or M^{A} linker (e.g., to an amino acid in the M^{A} linker) via a reactive group. Reactive groups are those to which an activated PEG molecule may be bound (e.g., a free amino or carboxyl group). In some embodiments, N-terminal amino acids and lysines (K) have a free amino group; and C-terminal amino acid residues have a free carboxyl group. Sulfhydryl groups (e.g., as found on cysteine residues) may also be used as a reactive group for attaching PEG.

In some embodiments, the PEG unit may be attached to the Multifunctional Linker or M^{A} linker (e.g., to an amino acid in the M^{A} linker) by using methoxylated PEG (“mPEG”) having different reactive moieties, including, but not limited to, succinimidyl succinate (SS), succinimidyl carbonate (SC), mPEG-imidate, para-nitrophenylcarbonate (NPC), succinimidyl propionate (SPA), and cyanuric chloride. Examples of mPEGs include, but are not limited to, mPEG-succinimidyl succinate (mPEG-SS), mPEG₂-succinimidyl succinate (mPEG₂-SS), mPEG-succinimidyl carbonate (mPEG-SC), mPEG₂-succinimidyl carbonate (mPEG₂-SC), mPEG-imidate, mPEG-para-nitrophenylcarbonate (mPEG-NPC), mPEG-imidate, mPEG₂-para-nitrophenylcarbonate (mPEG₂-NPC), mPEG-succinimidyl propionate (mPEG-SPA), mPEG₂-succinimidyl propionate (mPEG₂-SPA), mPEG-N-hydroxy-succinimide (mPEG-NHS), mPEG₂-N-hydroxy-succinimide (mPEG₂-NHS), mPEG-cyanuric chloride, mPEG₂-cyanuric chloride, mPEG₂-Lysinol-NPC, and mPEG₂-Lys-NHS. A wide variety of PEG species can be used, and substantially any suitable reactive PEG reagent can be used. In some embodiments, the reactive PEG reagent will result in formation of a carbamate or amide bond upon attachment to the Multifunctional Linker or M^{A} linker (e.g., to an amino acid in the M^{A} linker). The reactive PEG reagents include, but are not limited to, mPEG₂-N-hydroxy-succinimide (mPEG₂-NHS), bifunctional PEG propionaldehyde (mPEG₂-ALD), multi-Arm PEG, maleimide-containing PEG (mPEG(MAL)₂, mPEG₂(MAL)), mPEG-NH₂, mPEG-succinimidyl propionate (mPEG-SPA), succinimide of mPEG butanoate acid (mPEG-SBA), mPEG-thioesters, mPEG-Double Esters, mPEG-BTC, mPEG-ButyrALD, mPEG-acetaldehyde diethyl acetal (mPEG-ACET), heterofunctional PEGs (e.g., NH₂-PEG-COOH, Boc-PEG-NHS, Fmoc-PEG-NHS, NHS-PEG-vinylsulfone (NHS-PEG-VS), or NHS-PEG-MAL), PEG acrylates (ACRL-PEG-NHS), PEG-phospholipids (e.g., mPEG-DSPE), multi-armed PEGs of the SUNBRITE™ series including the glycerine-based PEGs activated by a chemistry chosen by those skilled in the art, any SUNBRITE activated PEGs (including but not limited to carboxyl-PEGs, p-NP-PEGs, Tresyl-PEGs, aldehyde PEGs, acetal-PEGs, amino-PEGs, thiol-PEGs, maleimido-PEGs, hydroxyl-PEG-amine, amino-PEG-COOK hydroxyl-PEG-aldehyde, carboxylic anhydride type-PEG, functionalized PEG-phospholipid, and other similar and/or suitable reactive PEGs.

In some embodiments, the PEG unit comprises at least 6 subunits, at least 7 subunits, at least 8 subunits, at least 9 subunits, at least 10 subunits, at least 11 subunits, at least 12 subunits, at least 13 subunits, at least 14 subunits, at least 15 subunits, at least 16 subunits, at least 17 subunits, at least 18 subunits, at least 19 subunits, at least 20 subunits, at least 21 subunits, at least 22 subunits, at least 23 subunits, or at least 24 subunits. In some such embodiments, the PEG unit comprises no more than about 72 subunits.

In some embodiments, the PEG unit comprises at least 6 subunits, at least 7 subunits, at least 8 subunits, at least 9 subunits, at least 10 subunits, at least 11 subunits, at least 12 subunits, at least 13 subunits, at least 14 subunits, at least 15 subunits, at least 16 subunits, at least 17 subunits, at least 18 subunits, at least 19 subunits, at least 20 subunits, at least 21 subunits, at least 22 subunits, at least 23 subunits, or at least 24 subunits.

In some embodiments, the PEG unit comprises at least 6 subunits, at least 7 subunits, at least 8 subunits, at least 9 subunits, at least 10 subunits, at least 11 subunits, at least 12 subunits, at least 13 subunits, at least 14 subunits, at least 15 subunits, at least 16 subunits, at least 17 subunits, or at least 18 subunits.

In some embodiments, the PEG unit comprises at least 6 subunits, at least 7 subunits, at least 8 subunits, at least 9 subunits, at least 10 subunits, at least 11 subunits, or at least 12 subunits.

In some embodiments, the PEG unit comprises at least 8 subunits, at least 9 subunits, at least 10 subunits, at least 11 subunits, or at least 12 subunits.

In some embodiments, the PEG unit comprises at least 6 subunits, at least 7 subunits, or at least 8 subunits.

In some embodiments, the PEG unit comprises one or more linear PEG chains each having at least 2 subunits, at least 3 subunits, at least 4 subunits, at least 5 subunits, at least 6 subunits, at least 7 subunits, at least 8 subunits, at least 9 subunits, at least 10 subunits, at least 11 subunits, at least 12 subunits, at least 13 subunits, at least 14 subunits, at least 15 subunits, at least 16 subunits, at least 17 subunits, at least 18 subunits, at least 19 subunits, at least 20 subunits, at least 21 subunits, at least 22 subunits, at least 23 subunits, or at least 24 subunits. In another embodiment, the PEG unit comprises a combined total of at least 6 subunits, at least 8, at least 10 subunits, or at least 12 subunits. In some such embodiments, the PEG unit comprises no more than a combined total of about 72 subunits, preferably no more than a combined total of about 36 subunits.

In some embodiments, the PEG unit comprises a combined total of from 4 to 72, 4 to 60, 4 to 48, 4 to 36 or 4 to 24 subunits, from 5 to 72, 5 to 60, 5 to 48, 5 to 36 or 5 to 24 subunits, from 6 to 72, 6 to 60, 6 to 48, 6 to 36 or from 6 to 24 subunits, from 7 to 72, 7 to 60, 7 to 48, 7 to 36 or 7 to 24 subunits, from 8 to 72, 8 to 60, 8 to 48, 8 to 36 or 8 to 24 subunits, from 9 to 72, 9 to 60, 9 to 48, 9 to 36 or 9 to 24 subunits, from 10 to 72, 10 to 60, 10 to 48, 10 to 36 or 10 to 24 subunits, from 11 to 72, 11 to 60, 11 to 48, 11 to 36 or 11 to 24 subunits, from 12 to 72, 12 to 60, 12 to 48, 12 to 36 or 12 to 24 subunits, from 13 to 72, 13 to 60, 13 to 48, 13 to 36 or 13 to 24 subunits, from 14 to 72, 14 to 60, 14 to 48, 14 to 36 or 14 to 24 subunits, from 15 to 72, 15 to 60, 15 to 48, 15 to 36 or 15 to 24 subunits, from 16 to 72, 16 to 60, 16 to 48, 16 to 36 or 16 to 24 subunits, from 17 to 72, 17 to 60, 17 to 48, 17 to 36 or 17 to 24 subunits, from 18 to 72, 18 to 60, 18 to 48, 18 to 36 or 18 to 24 subunits, from 19 to 72, 19 to 60, 19 to 48, 19 to 36 or 19 to 24 subunits, from 20 to 72, 20 to 60, 20 to 48, 20 to 36 or 20 to 24 subunits, from 21 to 72, 21 to 60, 21 to 48, 21 to 36 or 21 to 24 subunits, from 22 to 72, 22 to 60, 22 to 48, 22 to 36 or 22 to 24 subunits, from 23 to 72, 23 to 60, 23 to 48, 23 to 36 or 23 to 24 subunits, or from 24 to 72, 24 to 60, 24 to 48, 24 to 36 or 24 subunits.

In some embodiments, the PEG unit comprises one or more linear PEG chains having a combined total of from 4 to 72, 4 to 60, 4 to 48, 4 to 36 or 4 to 24 subunits, from 5 to 72, 5 to 60, 5 to 48, 5 to 36 or 5 to 24 subunits, from 6 to 72, 6 to 60, 6 to 48, 6 to 36 or 6 to 24 subunits, from 7 to 72, 7 to 60, 7 to 48, 7 to 36 or 7 to 24 subunits, from 8 to 72, 8 to 60, 8 to 48, 8 to 36 or 8 to 24 subunits, from 9 to 72, 9 to 60, 9 to 48, 9 to 36 or 9 to 24 subunits, from 10 to 72, 10 to 60, 10 to 48, 10 to 36 or 10 to 24 subunits, from 11 to 72, 11 to 60, 11 to 48, 11 to 36 or 11 to 24 subunits, from 12 to 72, 12 to 60, 12 to 48, 12 to 36 or 12 to 24 subunits, from 13 to 72, 13 to 60, 13 to 48, 13 to 36 or 13 to 24 subunits, from 14 to 72, 14 to 60, 14 to 48, 14 to 36 or 14 to 24 subunits, from 15 to 72, 15 to 60, 15 to 48, 15 to 36 or 15 to 24 subunits, from 16 to 72, 16 to 60, 16 to 48, 16 to 36 or 16 to 24 subunits, from 17 to 72, 17 to 60, 17 to 48, 17 to 36 or 17 to 24 subunits, from 18 to 72, 18 to 60, 18 to 48, 18 to 36 or 18 to 24 subunits, from 19 to 72, 19 to 60, 19 to 48, 19 to 36 or 19 to 24 subunits, from 20 to 72, 20 to 60, 20 to 48, 20 to 36 or 20 to 24 subunits, from 21 to 72, 21 to 60, 21 to 48, 21 to 36 or 21 to 24 subunits, from 22 to 72, 22 to 60, 22 to 48, 22 to 36 or 22 to 24 subunits, from 23 to 72, 23 to 60, 23 to 48, 23 to 36 or 23 to 24 subunits, or from 24 to 72, 24 to 60, 24 to 48, 24 to 36 or 24 subunits.

In some embodiments, the PEG unit is a derivatized linear single PEG chain having at least 2 subunits, at least 3 subunits, at least 4 subunits, at least 5 subunits, at least 6 subunits, at least 7 subunits, at least 8 subunits, at least 9 subunits, at least 10 subunits, at least 11 subunits, at least 12 subunits, at least 13 subunits, at least 14 subunits, at least 15 subunits, at least 16 subunits, at least 17 subunits, at least 18 subunits, at least 19 subunits, at least 20 subunits, at least 21 subunits, at least 22 subunits, at least 23 subunits, or at least 24 subunits.

In some embodiments, the PEG unit is a derivatized linear single PEG chain having from 6 to 72, 6 to 60, 6 to 48, 6 to 36 or 6 to 24 subunits, from 7 to 72, 7 to 60, 7 to 48, 7 to 36 or 7 to 24 subunits, from 8 to 72, 8 to 60, 8 to 48, 8 to 36 or 8 to 24 subunits, from 9 to 72, 9 to 60, 9 to 48, 9 to 36 or 9 to 24 subunits, from 10 to 72, 10 to 60, 10 to 48, 10 to 36 or 10 to 24 subunits, from 11 to 72, 11 to 60, 11 to 48, 11 to 36 or 11 to 24 subunits, from 12 to 72, 12 to 60, 12 to 48, 12 to 36 or 12 to 24 subunits, from 13 to 72, 13 to 60, 13 to 48, 13 to 36 or 13 to 24 subunits, from 14 to 72, 14 to 60, 14 to 48, 14 to 36 or 14 to 24 subunits, from 15 to 72, 15 to 60, 15 to 48, 15 to 36 or 15 to 24 subunits, from 16 to 72, 16 to 60, 16 to 48, 16 to 36 or 16 to 24 subunits, from 17 to 72, 17 to 60, 17 to 48, 17 to 36 or 17 to 24 subunits, from 18 to 72, 18 to 60, 18 to 48, 18 to 36 or 18 to 24 subunits, from 19 to 72, 19 to 60, 19 to 48, 19 to 36 or 19 to 24 subunits, from 20 to 72, 20 to 60, 20 to 48, 20 to 36 or 20 to 24 subunits, from 21 to 72, 21 to 60, 21 to 48, 21 to 36 or 21 to 24 subunits, from 22 to 72, 22 to 60, 22 to 48, 22 to 36 or 22 to 24 subunits, from 23 to 72, 23 to 60, 23 to 48, 23 to 36 or 23 to 24 subunits, or from 24 to 72, 24 to 60, 24 to 48, 24 to 36 or 24 subunits.

In some embodiments, the PEG unit is a derivatized linear single PEG chain having from 2 to 72, 2 to 60, 2 to 48, 2 to 36 or 2 to 24 subunits, from 2 to 72, 2 to 60, 2 to 48, 2 to 36 or 2 to 24 subunits, from 3 to 72, 3 to 60, 3 to 48, 3 to 36 or 3 to 24 subunits, from 3 to 72, 3 to 60, 3 to 48, 3 to 36 or 3 to 24 subunits, from 4 to 72, 4 to 60, 4 to 48, 4 to 36 or 4 to 24 subunits, from 5 to 72, 5 to 60, 5 to 48, 5 to 36 or 5 to 24 subunits.

In some embodiments, a linear PEG unit is:

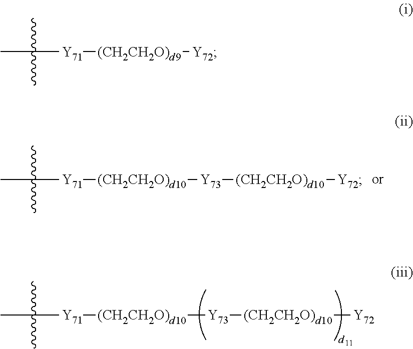

wherein;

the wavy line indicates site of attachment to the Multifunctional Linker or M^{A} linker (e.g., to an amino acid in the M^{A} linker);

Y₇₁ is a PEG attachment unit;

Y₇₂ is a PEG capping unit;

Y₇₃ is an PEG coupling unit (i.e., for coupling multiple PEG subunit chains together);

d₉ is an integer from 2 to 72, preferably from 4 to 72, more preferably from 6 to 72, from 8 to 72, from 10 to 72, from 12 to 72 or from 6 to 24;

each d₁₀ is independently an integer from 1 to 72.

d₁₁ is an integer from 2 to 5.

In some embodiments, there are at least 6, preferably at least 8, at least 10, or at least 12 PEG subunits in the PEG unit. In some embodiments, there are no more than 72 or 36 PEG subunits in the PEG unit.

In some embodiments, d₉ is 8 or about 8, 12 or about 12, 24 or about 24.

In some embodiments, each Y₇₂ is independently —C₁₋₁₀ alkyl, —C₂₋₁₀ alkyl-CO₂H, —C₂₋₁₀ alkyl-OH, —C₂₋₁₀ alkyl-NH₂, —C₂₋₁₀ alkyl-NH(C₁₋₃ alkyl), or C₂₋₁₀ alkyl-N(C₁₋₃ alkyl)₂.

In some embodiments, Y₇₂ is —C₁₋₁₀ alkyl, —C₂₋₁₀ alkyl-CO₂H, —C₂₋₁₀ alkyl-OH, or —C₂₋₁₀ alkyl-NH₂.

The PEG coupling unit is part of the PEG unit and is non-PEG material that acts to connect two or more chains of repeating CH₂CH₂O— subunits. In some embodiments, the PEG coupling unit Y₇₃ is —C₂₋₁₀ alkyl-C(O)—NH—, —C₂₋₁₀ alkyl-NH—C(O)—, —C₂₋₁₀ alkyl-NH—, —C₂₋₁₀ alkyl-C(O)—, —C₂₋₁₀ alkyl-O— or —C₂₋₄ alkyl-S—.

In some embodiments, each Y₇₃ is independently —C₁₋₁₀ alkyl-C(O)—NH—, —C₁₋₁₀ alkyl-NH—C(O)—, —C₂₋₁₀ alkyl-NH—, —C₂₋₁₀ alkyl-O—, —C₁₋₁₀ alkyl-S—, or —C₁₋₁₀ alkyl-NH—.

The PEG attachment unit is part of the PEG unit and acts to link the PEG unit to the Multifunctional Linker or M^{A} linker (e.g., to an amino acid in the M^{A} linker). In some embodiments, the amino acid has a functional group that forms a bond with the PEG Unit. Functional groups for attachment of the PEG unit to the amino acid include sulfhydryl groups to form disulfide bonds or thioether bonds, aldehyde, ketone, or hydrazine groups to form hydrazone bonds, hydroxylamine to form oxime bonds, carboxylic or amino groups to form peptide bonds, carboxylic or hydroxy groups to form ester bonds, sulfonic acids to form sulfonamide bonds, alcohols to form carbamate bonds, and amines to form sulfonamide bonds or carbamate bonds or amide bonds. Accordingly, the PEG unit can be attached to the amino acid, for example, via a disulfide, thioether, hydrazone, oxime, peptide, ester, sulfonamide, carbamate, or amide bond. Typically, the reaction for attaching the PEG unit can be a cycloaddition, addition, addition/elimination or substitution reaction, or a combination thereof when applicable.

In some embodiments, the PEG attachment unit Y₇₁ is a bond, —C(O)—, —O—, —S—, —S(O)—, —S(O)₂—, —NR₅—, —C(O)O—, —C(O)—C₁₋₁₀ alkyl, —C(O)—C₁₋₁₀ alkyl-O—, —C(O)—C₁₋₁₀ alkyl-CO₂—, —C(O)—C₁₋₁₀ alkyl-NR₅—, —C(O)—C₁₋₁₀ alkyl-S—, —C(O)—C₁₋₁₀ alkyl-C(O)—NR₅—, —C(O)—C₁₋₁₀ alkyl-NR₅—C(O)—, —C₁₋₁₀ alkyl, —C₁₋₁₀ alkyl-O—, —C₁₋₁₀ alkyl-CO₂—, —C₁₋₁₀ alkyl-NR₅—, —C₁₋₁₀ alkyl-S—, —C₁₋₁₀ alkyl-C(O)—NR₅—, —C₁₋₁₀ alkyl-NR₅—C(O)—, —CH₂CH₂SO₂—C₁₋₁₀ alkyl-, —CH₂C(O)—C₁₋₁₀ alkyl-, ═N—(O or N)—C₁₋₁₀ alkyl-O—, ═N—(O or N)—C₁₋₁₀ alkyl-NR₅—, ═N—(O or N)—C₁₋₁₀ alkyl-CO₂—, ═N—(O or N)—C₁₋₁₀-alkyl-S—,

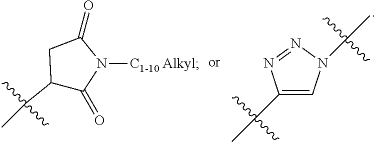

In some embodiments, Y₇₁ is —NH—, —C(O)—, a triazole group, —S—, or a maleimido-group such as

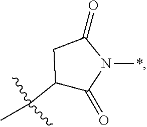

wherein the wavy line indicates attachment to the Multifunctional Linker or M^{A} linker (e.g., to an amino acid in the M^{A} linker) and the asterisk indicates the site of attachment within the PEG Unit.

Examples of linear PEG units include, but are not limited to:

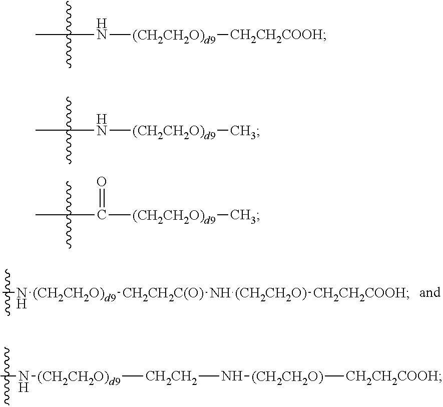

wherein the wavy line indicates site of attachment to the M^{A} linker (e.g., to an amino acid in the M^{A} linker), and each d₉ is independently an integer from 4 to 24, 6 to 24, 8 to 24, 10 to 24, 12 to 24, 14 to 24, or 16 to 24.

In some embodiments, d₉ is about 8, about 12, or about 24.

In some embodiments, the PEG unit is from about 300 daltons to about 5 kilodaltons; from about 300 daltons, to about 4 kilodaltons; from about 300 daltons, to about 3 kilodaltons; from about 300 daltons, to about 2 kilodaltons; or from about 300 daltons, to about 1 kilodalton. In some such aspects, the PEG unit has at least 6 subunits or at least 8, 10 or 12 subunits. In some embodiments, the PEG unit has at least 6 subunits or at least 8, 10 or 12 subunits but no more than 72 subunits, preferably no more than 36 subunits.

Suitable polyethylene glycols may have a free hydroxy group at each end of the polymer molecule, or may have one hydroxy group etherified with a lower alkyl, e.g., a methyl group. Also suitable for the practice of the disclosure are derivatives of polyethylene glycols having esterifiable carboxy groups. Polyethylene glycols are commercially available under the trade name PEG, usually as mixtures of polymers characterized by an average molecular weight. Polyethylene glycols having an average molecular weight from about 300 to about 5000 are preferred, those having an average molecular weight from about 600 to about 1000 being particularly preferred.

Other examples of hydrophilic groups that are suitable for the conjugates, scaffolds, and methods disclosed herein can be found in e.g., U.S. Pat. No. 8,367,065 column 13; U.S. Pat. No. 8,524,696 column 6; WO2015/057699 and WO 2014/062697, the contents of each of which are hereby incorporated by reference in their entireties.

### Antibody-Drug Conjugate (ADC) Type II

In some embodiments, the conjugate (e.g., the antibody-drug conjugate (ADC)) of the present disclosure is of Formula (III):

PBRM-(A¹ₐ₆-L¹ₛ₂-L²_{y1}-D)_{d13} (III)

or pharmaceutically acceptable salt or solvate thereof, wherein:

PBRM denotes a protein based recognition-molecule;

each occurrence of D is independently a PBD drug moiety;

A¹ is a stretcher unit;

a₆ is an integer 1 or 2;

L^{P} is a specificity unit;

s₂ is an integer from about 0 to about 12;

L² is a spacer unit;

y₁ is an integer from 0 to 2; and

d₁₃ is an integer from about 1 to about 14.

In some embodiments, the conjugates of Formula (III) include those where each of the moieties defined for one of PBRM, D, A¹, a₆, L¹, s₂, L², y₁, and d₁₃ can be combined with any of the moieties defined for the others of PBRM, D, A¹, a₆, L¹, s₂, L², y₁, and d₁₃.

In some embodiments, the conjugate (e.g., the antibody-drug conjugate (ADC)) of the present disclosure is of Formula (IIIa) or (IIIb):

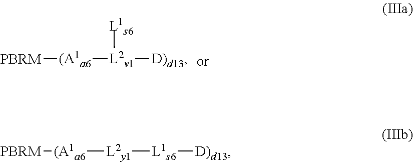

or a pharmaceutically acceptable salt or solvate thereof,
wherein:

PBRM denotes a protein based recognition-molecule;

each occurrence of D is independently a PBD drug moiety;

A¹ is a stretcher unit linked to the spacer unit L²;

a₆ is an integer 1 or 2;

L^{P} is a specificity unit linked to the spacer unit L²;

s₆ is an integer from about 0 to about 12.

L² is a spacer unit;

y₁ is an integer 0, 1 or 2; and

d₁₃ is an integer from about 1 to about 14.

In some embodiments, the conjugates of any one of Formulae (IIIa)-(IIIb) include those where each of the moieties defined for one of PBRM, D, A¹, a₆, L¹, s₆, L², y₁, and d₁₃ can be combined with any of the moieties defined for the others of PBRM, D, A¹, a₆, L¹, s₆, L², y₁, and d₁₃.

In some embodiments, the conjugate (e.g., the antibody-drug conjugate (ADC)) of the present disclosure is of any one of Formulae (IIIc) to (IIIf):

PBRM-(A¹ₐ₆-L¹ₛ₂-L²_{y1}-D)_{d13}, (IIIc)

PBRM-(A¹ₐ₆-L¹ₛ₂-D)_{d13}, (IIId)

PBRM-(A¹-L¹-D)_{d13}, (IIIe)

PBRM-(A¹-D)_{d13}, or (IIIf)

or a pharmaceutically acceptable salt or solvate thereof, wherein PBRM, A¹, a₆, L¹ s₂, L², y₁, D, and d₁₃ are as defined herein.

In some embodiments, the conjugates of any one of Formulae (IIIc)-(IIIf) include those where each of the moieties defined for one of PBRM, A¹, a₆, L¹ s₂, L², y₁, D, and d₁₃ can be combined with any of the moieties defined for the others of PBRM, A¹, a₆, L¹ s₂, L², y₁, D, and d₁₃.

In some embodiments, the PBRM specifically binds to a target molecule on the surface of a target cell. An exemplary formula is:

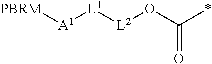

wherein the asterisk indicates the point of attachment to the Drug moiety (D), PRBM is targeting moiety, L¹ is a Specificity unit, A¹ is a Stretcher unit connecting L¹ to the PBRM, L² is a Spacer unit, which is a covalent bond, a self-immolative group or together with —OC(═O)— forms a self-immolative group, and L² is optional. —OC(═O)— may be considered as being part of L¹ or L², as appropriate.

In some embodiments, the PBRM specifically binds to a target molecule on the surface of a target cell. An exemplary formula is:

PBRM-A¹ₐ₆-L¹ₛ₆-L²_{y1}-*

wherein the asterisk indicates the point of attachment to the Drug moiety (D), PBRM is the targeting moiety, L¹ is a Specificity unit, A¹ is a Stretcher unit connecting L¹ to the PBRM, L² is a Spacer unit which is a covalent bond or a self-immolative group, and a₆ is an integer 1 or 2, s₆ is an integer 0, 1 or 2, and y₁ is an integer 0, 1 or 2.

In the embodiments above, L¹ can be a cleavable Specificity unit, and may be referred to as a “trigger” that when cleaved activates a self-immolative group (or self-immolative groups) L², when a self-immolative group(s) is present. When the Specificity unit L¹ is cleaved, or the linkage (i.e., the covalent bond) between L¹ and L² is cleaved, the self-immolative group releases the PBD Drug moiety (D).

In some embodiments, the PBRM specifically binds to a target molecule on the surface of a target cell. An exemplary formula is:

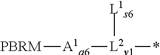

wherein the asterisk indicates the point of attachment to the PBD Drug moiety (D), PBRM is the targeting moiety, L¹ is a Specificity unit connected to L², A¹ is a Stretcher unit connecting L² to the PBRM, L² is a self-immolative group, and a₆ is an integer 1 or 2, s₆ is an integer 0, 1 or 2, and y₁ is an integer 0, 1 or 2.

In the various embodiments discussed herein, the nature of L¹ and L² can vary widely. These groups are chosen on the basis of their characteristics, which may be dictated in part, by the conditions at the site to which the conjugate is delivered. Where the Specificity unit L¹ is cleavable, the structure and/or sequence of L¹ is selected such that it is cleaved by the action of enzymes present at the target site (e.g., the target cell). L¹ units that are cleavable by changes in pH (e.g. acid or base labile), temperature or upon irradiation (e.g. photolabile) may also be used. L¹ units that are cleavable under reducing or oxidizing conditions may also find use in the conjugates of the present disclosure.

In some embodiments, L¹ may comprise one amino acid or a contiguous sequence of amino acids. The amino acid sequence may be the target substrate for an enzyme.

In some embodiments, L¹ is cleavable by the action of an enzyme. In one embodiment, the enzyme is an esterase or a peptidase. In some embodiments, L¹ may be cleaved by a lysosomal protease, such as, for example, a cathepsin.

In some embodiments, L² is present and together with —C(═O)O— forms a self-immolative group or self-immolative groups. In some embodiments, —C(═O)O— also is a self-immolative group.

In some embodiments, where L¹ is cleavable by the action of an enzyme and L² is present, the enzyme cleaves the bond between L¹ and L², whereby the self-immolative group(s) release the Drug moiety.

In some embodiments, L¹ and L², where present, may be connected by a bond selected from: (i) —C(═O)NH; (ii) —C(═O)O—; (iii) —NHC(═O)—; (iv) —OC(═O)—; (v) —OC(═O)O—; (vi) —NHC(═O)O—; (vii) —OC(═O)NH—; (viii) —NHC(═O)NH—; and (ix) —O— (a glycosidic bond).

In some embodiments, an amino group of L¹ that connects to L² may be the N-terminus of an amino acid or may be derived from an amino group of an amino acid side chain, for example a lysine amino acid side chain.

In some embodiments, a carboxyl group of L¹ that connects to L² may be the C-terminus of an amino acid or may be derived from a carboxyl group of an amino acid side chain, for example a glutamic acid amino acid side chain.

In some embodiments, a hydroxy group of L¹ that connects to L² may be derived from a hydroxy group of an amino acid side chain, such as, for example, a serine amino acid side chain.

In some embodiments, —C(═O)O— and L² together form the group:

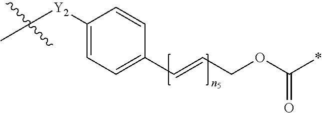

wherein the asterisk indicates the point of attachment to the Drug moiety, the wavy line indicates the point of attachment to the L¹, Y₂ is —N(H)—, —O—, —C(═O)N(H)— or —C(═O)O—, and n₅ is an integer from 0 to 3. The phenylene ring is optionally substituted with one, two or three substituents as described herein.

In some embodiments, Y₂ is NH.

In some embodiments, n₅ is 0 or 1. Preferably, n₅ is 0.

In some embodiments, when Y₂ is NH and n₅ is 0, the self-immolative group may be referred to as a p-aminobenzylcarbonyl linker (PABC). The self-immolative group will allow for release of the Drug moiety (i.e., the PBD) when a remote site in the linker is activated, proceeding along the lines as shown below (for n₅=0):

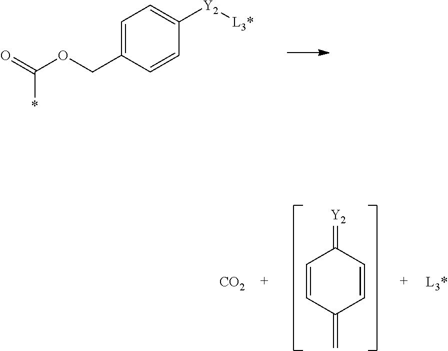

wherein the asterisk indicates the attachment to the Drug, U is the activated form of the remaining portion of the linker and the released Drug moiety is not shown. These groups have the advantage of separating the site of activation from the Drug.

In some embodiments, —C(═O)O— and L together form a group selected from:

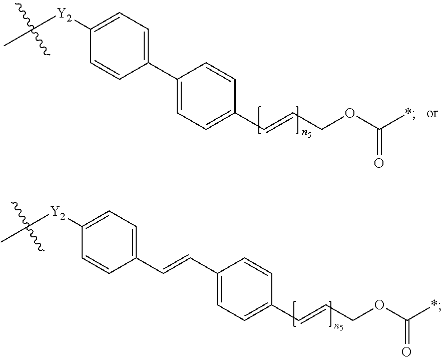

wherein the asterisk, the wavy line, Y₂, and n₅ are as defined above. Each phenylene ring is optionally substituted with one, two or three substituents as described herein. In one embodiment, the phenylene ring having the Y₁ substituent is optionally substituted and the phenylene ring not having the Y₁ substituent is unsubstituted.

In some embodiments, —C(═O)O— and L² together form a group selected from:

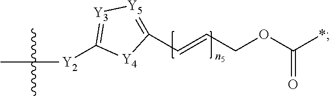

wherein the asterisk, the wavy line, Y₂, and n₅ are as defined herein, Y₄ is 0, S or NR, Y₃ is N, CH, or CR, and Y₅ is N, CH, or CR.

In some embodiments, Y₃ is N.

In some embodiments, Y₃ is CH.

In some embodiments, Y₄ is O or S.

In some embodiments, Y₅ is CH.

In some embodiments, the covalent bond between L and L² is a cathepsin labile (e.g., cleavable) bond.

In some embodiments, L¹ comprises a dipeptide. The amino acids in the dipeptide may be any combination of natural amino acids and non-natural amino acids. In some embodiments, the dipeptide comprises natural amino acids. When the linker is a cathepsin labile linker, the dipeptide is the site of action for cathepsin-mediated cleavage. The dipeptide then is a recognition site for cathepsin.

In some embodiments, the group —X₅—X₆— in dipeptide, —NH—X₅—X₆—CO—, is selected from: (i) -Phe-Lys-; (ii) -Val-Ala; (iii) -Val-Lys-; (iv) -Ala-Lys; (v) -Ala-Ala; (vi) -Val-Cit; (vii) -Phe-Cit; (viii) -Leu-Cit; (ix) -Ile-Cit-Phe-Arg-, and (x) -Trp-Cit-; wherein Cit is citrulline. In such a dipeptide, —NH— is the amino group of X₅, and CO is the carbonyl group of X₆.

In some embodiments, the group —X₅—X₆— in dipeptide, is selected from: (i) -Phe-Lys-, (ii) -Val-Ala-, (iii) -Ala-Ala-, (iv) -Val-Lys-, (v) -Ala-Lys-, and (vi) -Val-Cit-.

In some embodiments, the group —X₅—X₆— in dipeptide, is -Phe-Lys-, Val-Cit, -Ala-Ala- or -Val-Ala-.

Other dipeptide combinations of interest include: (i) -Gly-Gly-, (ii) -Pro-Pro-, and (iii) -Val-Glu-.

Other dipeptide combinations may be used, including those described by Dubowchik et al., which is incorporated herein by reference.

In some embodiments, the amino acid side chain is chemically protected, where appropriate. The side chain protecting group may be a group as discussed below. Protected amino acid sequences are cleavable by enzymes. In some embodiments, a dipeptide sequence comprising a Boc side chain-protected Lys residue is cleavable by cathepsin.

Protecting groups for the side chains of amino acids are well known in the art and are described in the Novabiochem Catalog. Additional protecting group strategies are set out in Protective groups in Organic Synthesis, Greene and Wuts.

Possible side chain protecting groups are amino acids having reactive side chain functionality, such as, for example:

(i) Arg: Z, Mtr, Tos;

(ii) Asn: Trt, Xan;

(iii) Asp: Bzl, t-Bu;

(iv) Cys: Acm, Bzl, Bzl-OMe, Bzl-Me, Trt;

(v) Glu: Bzl, t-Bu; Gin: Trt, Xan;

(vi) His: Boc, Dnp, Tos, Trt;

(vii) Lys: Boc, Z—CI, Fmoc, Z;

(viii) Ser: Bzl, TBDMS, TBDPS;

(ix) Thr: Bz;

(x) Trp: Boc; or

(xi) Tyr: Bzl, Z, Z—Br.

In some embodiments, —X₆— is connected indirectly to the Drug moiety. In such an embodiment, the Spacer unit L₂ is present.

In some embodiments, the dipeptide is used in combination with a self-immolative group(s) (the Spacer unit). The self-immolative group(s) may be connected to —X₆—.

When a self-immolative group is present, —X₆— is connected directly to the self-immolative group. In one embodiment, —X₆— is connected to the group Y₂ of the self-immolative group. Preferably the group —X₆—CO— is connected to Y₂, wherein Y₂ is NH.

In some embodiments, —X₅ is connected directly to A¹. Preferably the group NH—X₅— (the amino terminus of X₈) is connected to A. A¹ may comprise the functionality —CO— thereby to form an amide link with —X₅.

In some embodiments, L¹ and L² together with —OC(═O)— comprise the group —X₅— X₆-PABC-. The PABC group is connected directly to the Drug moiety. In one example, the self-immolative group and the dipeptide together form the group -Phe-Lys-PABC-, is:

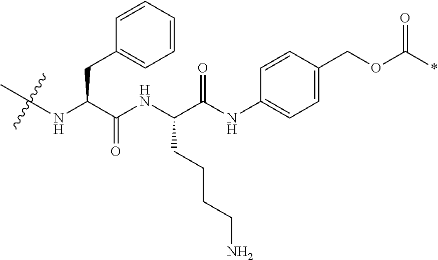 [SMILES: *N[C@@H](Cc1ccccc1)C(=O)N[C@@H](CCCCN)C(=O)Nc1ccc(COC(*)=O)cc1]

wherein the asterisk indicates the point of attachment to the Drug moiety, and the wavy line indicates the point of attachment to the remaining portion of L¹ or the point of attachment to A¹. In some embodiments, the wavy line indicates the point of attachment to A¹.

In some embodiments, the self-immolative group and the dipeptide together form the group -Val-Ala-PABC- or -Ala-Ala-PABC are:

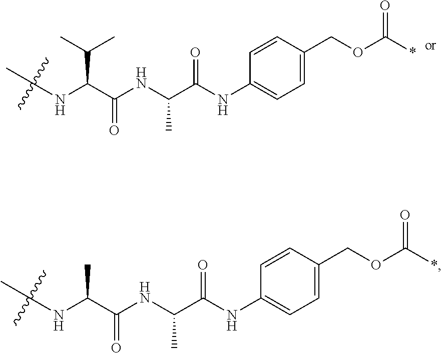

wherein the asterisk and the wavy line are as defined above.

In some embodiments, L¹ and L² together with —OC(═O)— are:

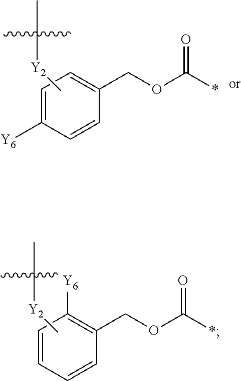

wherein the asterisk indicates the point of attachment to the Drug moiety, the wavy line indicates the point of attachment to A¹, Y₂ is a covalent bond or a functional group, and Y₆ is a group that is susceptible to cleavage thereby to activate a self-immolative group.

In some embodiments, Y₆ is selected such that the group is susceptible to cleavage, e.g., by light or by the action of an enzyme. In some embodiments, Y₆ may be —NO₂ or glucuronic acid (e.g., β-glucuronic acid). The former may be susceptible to the action of a nitroreductase, the latter to the action of a β-glucuronidase.

In some embodiments, the group Y₂ may be a covalent bond.

In some embodiments, the group Y₂ may be a functional group selected from (i) —C(═O)—; (ii) —NH—; (iii) —O—; (iv) —C(═O)NH—; (v) —C(═O)O—; (vi) —NHC(═O)—; (vii) —OC(═O)—; (viii) —OC(═O)O—; (ix) —NHC(═O)O—; (x) —OC(═O)NH—; (xi) —NHC(═O)NH—; (xii) —NHC(═O)NH; (xiii) —C(═O)NHC(═O)—; (xiv) SO₂; and (v) —S—.

In some embodiments, the group Y₂ is preferably —NH—, —CH₂—, —O—, and —S—.

In some embodiments, L¹ and L² together with —OC(═O)— is:

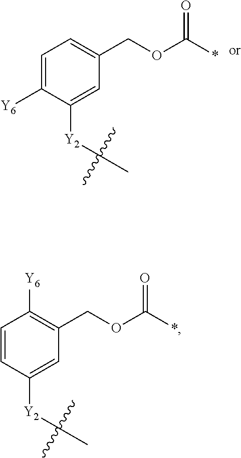

wherein the asterisk indicates the point of attachment to the Drug moiety, the wavy line indicates the point of attachment to A¹, Y₂ is a covalent bond or a functional group and Y₆ is glucuronic acid (e.g., β-glucuronic acid). Y₂ is preferably a functional group selected from —NH—.

In some embodiments, L¹ and L² together are:

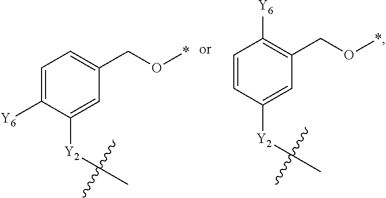

wherein the asterisk indicates the point of attachment to the remainder of L² or the Drug moiety, the wavy line indicates the point of attachment to A¹, Y₂ is a covalent bond or a functional group and Y₆ is glucuronic acid (e.g., β-glucuronic acid). Y₂ is preferably a functional group selected from —NH—, —CH₂—, —O—, and —S—.

In some embodiments, Y₂ is a functional group as set forth above, the functional group is linked to an amino acid, and the amino acid is linked to the Stretcher unit A¹. In some embodiments, amino acid is β-alanine. In such an embodiment, the amino acid is equivalently considered part of the Stretcher unit.

In some embodiments, the Specificity unit L¹ and the PBRM are indirectly connected via the Stretcher unit.

In some embodiments, L¹ and A¹ may be connected by a bond selected from: (i) —C(═O)NH—; (ii) —C(═O)O—; (iii) —NHC(═O)—; (iv) —OC(═O)—; (v) —OC(═O)O—; (vi) —NHC(═O)O—; (vii) —OC(═O)NH—; and (viii) —NHC(═O)NH—.

In some embodiments, the group A¹ is:

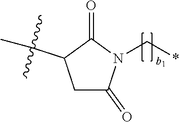

wherein the asterisk indicates the point of attachment to L¹, the wavy line indicates the point of attachment to the PRBM moiety, and b₁ is an integer from 0 to 6. In one embodiment, b₁ is 5.

In some embodiments, the group A¹ is:

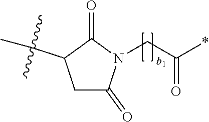

wherein the asterisk indicates the point of attachment to L¹, the wavy line indicates the point of attachment to the PRBM moiety, and b₁ is an integer from 0 to 6. In one embodiment, b₁ is 5.

In some embodiments the group A is:

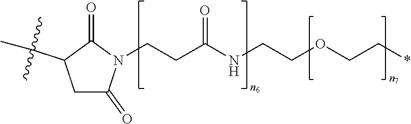

wherein the asterisk indicates the point of attachment to L¹, the wavy line indicates the point of attachment to the PBRM moiety, n₆ is an integer 0 or 1, and n₇ is an integer from 0 to 30. In a preferred embodiment, n₆ is 1 and n₇ is 0 to 10, 1 to 8, preferably 4 to 8, most preferably 4 or 8.

In some embodiments, the group A¹ is:

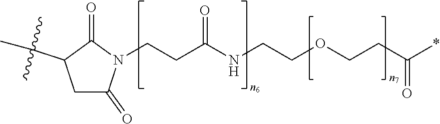

wherein the asterisk indicates the point of attachment to L¹, the wavy line indicates the point of attachment to the PBRM moiety, n₆ is an integer 0 or 1, and n₇ is an integer from 0 to 30. In a preferred embodiment, n₆ is 1 and n₇ is 0 to 10, 1 to 8, preferably 4 to 8, most preferably 4 or 8.

In some embodiments, the group A¹ is:

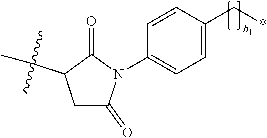

wherein the asterisk indicates the point of attachment to L¹, the wavy line indicates the point of attachment to the PBRM moiety, and b₁ is an integer from 0 to 6. In one embodiment, b₁ is 5.

In some embodiments, the group A¹ is:

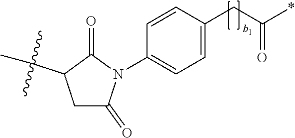

wherein the asterisk indicates the point of attachment to L, the wavy line indicates the point of attachment to the PBRM moiety, and b₁ is an integer from 0 to 6. In one embodiment, b₁ is 5.

In some embodiments, the group A¹ is:

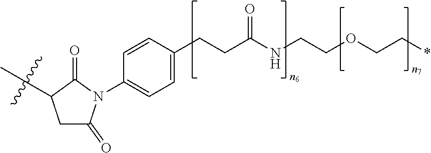

wherein the asterisk indicates the point of attachment to L¹, the wavy line indicates the point of attachment to the PBRM moiety, n₆ is an integer 0 or 1, and n₇ is an integer from 0 to 30. In a preferred embodiment, n₆ is 1 and n₇ is 0 to 10, 1 to 8, preferably 4 to 8, most preferably 4 or 8.

In some embodiments, the group A¹ is:

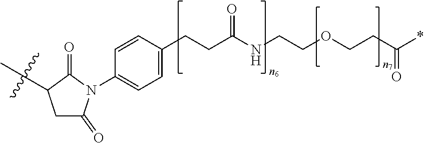

wherein the asterisk indicates the point of attachment to L¹, the wavy line indicates the point of attachment to the PBRM moiety, n₆ is an integer 0 or 1, and n₇ is an integer from 0 to 30. In a preferred embodiment, n₆ is 1 and n₇ is 0 to 10, 1 to 8, preferably 4 to 8, most preferably 4 or 8.

In some embodiments, the connection between the PBRM moiety and A¹ is through a thiol residue of the PBRM moiety and a maleimide group of A¹.

In some embodiments, the connection between the PBRM moiety and A¹ is:

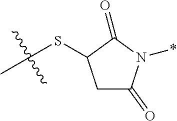 [SMILES: *SC1CC(=O)N(*)C1=O]

wherein the asterisk indicates the point of attachment to the remaining portion of A¹, L¹, L² or D, and the wavy line indicates the point of attachment to the remaining portion of the PBRM moiety. In this embodiment, the S atom is typically derived from the PBRM moiety.

In each of the embodiments above, an alternative functionality may be used in place of the malemide-derived group is:

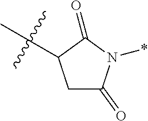 [SMILES: *C1CC(=O)N(*)C1=O]

wherein the wavy line indicates the point of attachment to the PBRM moiety as before, and the asterisk indicates the bond to the remaining portion of the A¹ group, or to L¹, L² or D.

In some embodiments, the maleimide-derived group is replaced with the group:

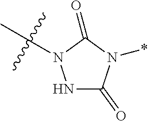 [SMILES: *n1[nH]c(=O)n(*)c1=O]

wherein the wavy line indicates point of attachment to the PBRM moiety, and the asterisk indicates the bond to the remaining portion of the A¹ group, or to L¹, L² or D.

In some embodiments, the maleimide-derived group is replaced with a group, which optionally together with a PBRM moiety (e.g., a PBRM), is selected from: (i) —C(═O)NH—; (ii) —C(═O)O—; (iii) —NHC(═O)—; (iv) —OC(═O)—; (v) —OC(═O)O—; (vi) —NHC(═O)O—; (vii) —OC(═O)NH—; (viii) —NHC(═O)NH—; (ix) —NHC(═O)NH; (x) —C(═O)NHC(═O)—; (xi) —S—; (xii) —S—S—; (xiii) —CH₂C(═O)—; (xiv) —C(═O)CH₂—; (xv)=N—NH—; and (xvi) —NH—N═. Of these —C(═O)CH₂— may be preferred especially when the carbonyl group is bound to —NH—.

In some embodiments, the maleimide-derived group is replaced with a group, which optionally together with the PBRM moiety, is selected from:

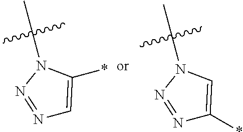 [SMILES: *c1cnnn1* ; *c1cn(*)nn1]

wherein the wavy line indicates either the point of attachment to the PBRM moiety or the bond to the remaining portion of the A¹ group, and the asterisk indicates the other of the point of attachment to the PBRM moiety or the bond to the remaining portion of the A¹ group.

Other groups suitable for connecting L¹ to the PBRM are described in WO 2005/082023.

In some embodiments, the Stretcher unit A¹ is present, the Specificity unit L is present and Spacer unit L² is absent. Thus, L¹ and the Drug moiety are directly connected via a bond. Equivalently in this embodiment, L₂ is a bond.

In some embodiments, L¹ and D may be connected by a bond selected from: (i) —C(═O)N<; (ii) —C(═O)O—; (iii) —NHC(═O)—; (iv) —OC(═O)—; (v) —OC(═O)O—; (vi) —NHC(═O)O; (vii) —OC(═O)N<; and (viii) —NHC(═O)N<; wherein N< or O— are part of D.

In some embodiments, L¹ and D are preferably connected by a bond selected from: —C(═O)N<, and —NHC(═O)—.

In some embodiments, L¹ comprises a dipeptide and one end of the dipeptide is linked to D. As described above, the amino acids in the dipeptide may be any combination of natural amino acids and non-natural amino acids. In some embodiments, the dipeptide comprises natural amino acids. Where the linker is a cathepsin labile linker, the dipeptide is the site of action for cathepsin-mediated cleavage. The dipeptide then is a recognition site for cathepsin.

In some embodiments, the group —X₅—X₆— in dipeptide, —NH—X₅—X₆—CO—, is selected from: (i) -Phe-Lys-; (ii) -Val-Ala-; (iii) -Ala-Ala-; (iv) -Val-Lys-; (v) -Ala-Lys-; (vi) -Val-Cit-; (vii) -Phe-Cit-; (viii) -Leu-Cit-; (ix) -Ile-Cit-; (x) -Phe-Arg-; and (xi) -Trp-Cit-; wherein Cit is citrulline. In such a dipeptide, —NH— is the amino group of X₈, and CO is the carbonyl group of X₆.

In some embodiments, the group —X₅—X₆— in dipeptide, —NH—X₅—X₆—CO—, is selected from: (i) -Phe-Lys-; (ii) -Val-Ala-; (iii) -Ala-Ala-; (iv) -Val-Lys-; (v) -Ala-Lys-; and (vi) -Val-Cit-.

In some embodiments, the group —X X₂— in dipeptide, is -Phe-Lys-, -Ala-Ala- or -Val-Ala-.

Other dipeptide combinations of interest include: (i) -Gly-Gly-; (ii) -Pro-Pro-; and (iii) -Val-Glu-.

Other dipeptide combinations may be used, including those described above.

In some embodiments, L¹-D is:

—NH—X₅—X₆—CO—N<*

wherein —NH—X₅—X₆—CO— is the dipeptide, —N< is part of the Drug moiety, the asterisk indicates the points of attachment to the remainder of the Drug moiety, and the wavy line indicates the point of attachment to the remaining portion of L¹ or the point of attachment to A. Preferably, the wavy line indicates the point of attachment to A¹.

In some embodiments, the dipeptide is valine-alanine and L¹-D is:

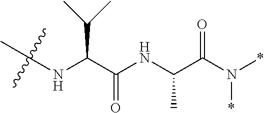 [SMILES: *N[C@H](C(=O)N[C@@H](C)C(=O)N(*)*)C(C)C]

wherein the asterisks, —N< and the wavy line are as defined above.

In some embodiments, the dipeptide is alanine-alanine and L¹-D is:

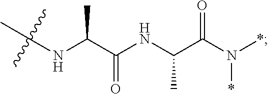

wherein the asterisks, —N< and the wavy line are as defined above.

In some embodiments, the dipeptide is phenylalnine-lysine and L¹-D is:

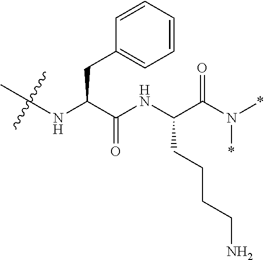 [SMILES: *N[C@@H](Cc1ccccc1)C(=O)N[C@@H](CCCCN)C(=O)N(*)*]

wherein the asterisks, —N< and the wavy line are as defined above.

In some embodiments, the dipeptide is valine-citrulline.

In some embodiments, the groups A¹-L¹ are:

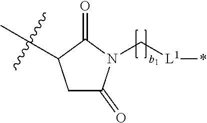

wherein the asterisk indicates the point of attachment to L² or D, the wavy line indicates the point of attachment to the PBRM moiety, and b₁ is an integer from 0 to 6. In some embodiments, b₁ is 5.

In some embodiments, the groups A¹-L¹ are:

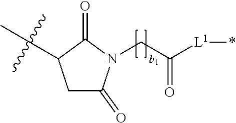

wherein the asterisk indicates the point of attachment to L² or D, the wavy line indicates the point of attachment to the PBRM moiety, and b₁ is an integer from 0 to 6. In some embodiments, b₁ is 5.

In some embodiments, the groups A¹-L¹ are:

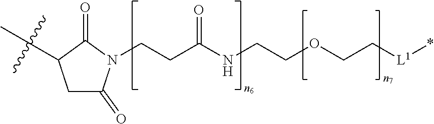

wherein the asterisk indicates the point of attachment to L² or D, the wavy line indicates the point of attachment to the PBRM moiety, n₆ is an integer 0 or 1, and n₇ is an integer from 0 to 30. In a preferred embodiment, n₆ is 1 and n₇ is 0 to 10, 1 to 8, preferably 4 to 8, most preferably 4 or 8.

In some embodiments, the groups A¹-L¹ are:

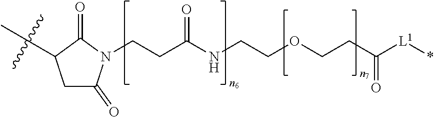

wherein the asterisk indicates the point of attachment to L² or D, the wavy line indicates the point of attachment to the PBRM moiety, n₆ is an integer 0 or 1, and n₇ is an integer from 0 to 30. In a preferred embodiment, n₆ is 1 and n₇ is 0 to 10, 1 to 7, preferably 3 to 7, most preferably 3 or 7. one embodiment, the groups A1

In some embodiments, the groups A¹-L¹ are:

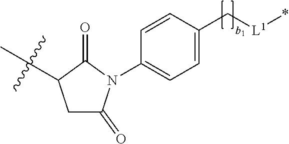

wherein the asterisk indicates the point of attachment to L² or D, the wavy line indicates the point of attachment to the PBRM moiety, and b₁ is an integer from 0 to 6. In some embodiments, b₁ is 5.

In some embodiments, the groups A¹-L¹ are:

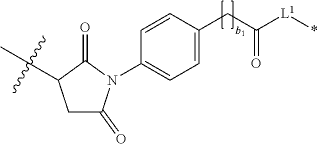

wherein the asterisk indicates the point of attachment to L² or D, the wavy line indicates the point of attachment to the PBRM moiety, and b₁ is an integer from 0 to 6. In some embodiments, b₁ is 5.

In some embodiments, the groups A¹-L¹ are:

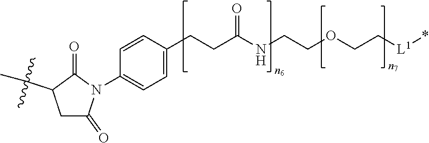

wherein the asterisk indicates the point of attachment to L² or D, the wavy line indicates the point of attachment to the PBRM moiety, n₆ is an integer 0 or 1, and n₇ is an integer from 0 to 30. In a preferred embodiment, n₆ is 1 and n₇ is 0 to 10, 1 to 8, preferably 4 to 8, most preferably 4 or 8.

In some embodiments, the groups A¹-L¹ are:

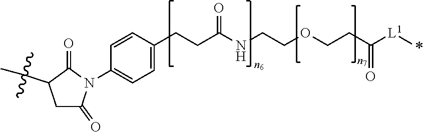

wherein the asterisk indicates the point of attachment to L² or D, the wavy line indicates the point of attachment to the PBRM moiety, n₆ is an integer 0 or 1, and n₇ is an integer from 0 to 30. In a preferred embodiment, n₆ is 1 and n₇ is 0 to 10, 1 to 8, preferably 4 to 8, most preferably 4 or 8.

In some embodiments, the groups PBRM-A¹-L¹ are:

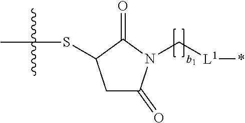

wherein the asterisk indicates the point of attachment to L² or D, S is a sulfur group of the PBRM moiety, the wavy line indicates the point of attachment to the rest of the PBRM moiety, and b₁ is an integer from 0 to 6. In some embodiments, b₁ is 5.

In some embodiments, the group PBRM-A¹-L¹ are:

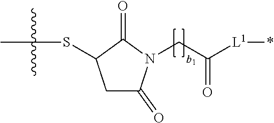

wherein the asterisk indicates the point of attachment to L₂ or D, S is a sulfur group of the PBRM moiety, the wavy line indicates the point of attachment to the remainder of the PBRM moiety, and b₁ is an integer from 0 to 6. In some embodiments, b₁ is 5.

In some embodiments, the groups PBRM-A¹-L¹ are:

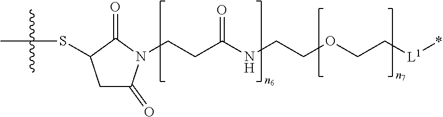

wherein the asterisk indicates the point of attachment to L² or D, S is a sulfur group of the PBRM moiety, the wavy line indicates the point of attachment to the remainder of the PBRM moiety, n₆ is an integer 0 or 1, and n₇ is an integer from 0 to 30. In a preferred embodiment, n₆ is 1 and n₇ is 0 to 10, 1 to 8, preferably 4 to 8, most preferably 4 or 8.

In some embodiments, the groups PBRM-A¹-L¹ are:

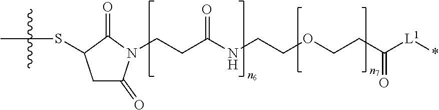

wherein the asterisk indicates the point of attachment to L² or D, S is a sulfur group of the PBRM moiety, the wavy line indicates the point of attachment to the remainder of the PBRM moiety, n₆ is an integer 0 or 1, and n₇ is an integer from 0 to 30. In a preferred embodiment, n₆ is 1 and n₇ is 0 to 10, 1 to 8, preferably 4 to 8, most preferably 4 or 8.

In some embodiments, the groups PBRM-A¹-L¹ are:

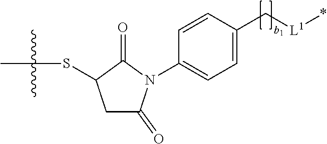

wherein the asterisk indicates the point of attachment to L. or D, S is a sulfur group of the PBRM moiety, the wavy line indicates the point of attachment to the remainder of the PBRM moiety, b₁ is an integer from 0 to 6. In some embodiments, b₁ is 5.

In some embodiments, the groups PBRM-A¹-L¹ are:

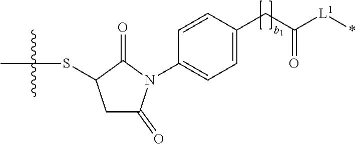

wherein the asterisk indicates the point of attachment to L₂ or D, S is a sulfur group of the PBRM moiety, the wavy line indicates the point of attachment to the remainder of the PBRM moiety, b₁ is an integer from 0 to 6. In some embodiments, b₁ is 5.

In some embodiments, the groups PBRM-A¹-L¹ are:

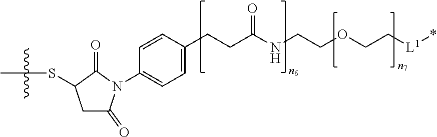

wherein the asterisk indicates the point of attachment to L² or D, S is a sulfur group of the PBRM moiety, the wavy line indicates the point of attachment to the remainder of the PBRM moiety, b₁ is an integer from 0 to 6. In some embodiments, b₁ is 5.

In some embodiments, the groups PBRM-A¹-L¹ are:

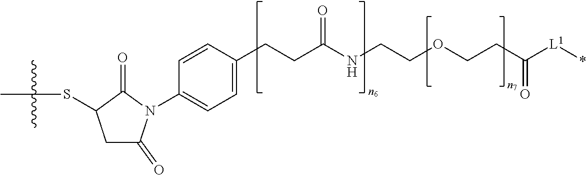

wherein the asterisk indicates the point of attachment to L² or D, S is a sulfur group of the PBRM moiety, the wavy line indicates the point of attachment to the remainder of the PBRM moiety, b₁ is an integer from 0 to 6. In some embodiments, b₁ is 5.

In some embodiments, the Stretcher unit is an acetamide unit, having the formula:

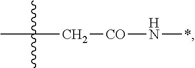

wherein the asterisk indicates the point of attachment to the remainder of the Stretcher unit, L¹ or D, and the wavy line indicates the point of attachment to the PBRM moiety.

### Linker-Drugs

In other embodiments, Linker-Drug compounds are provided for conjugation to a PBRM moiety. In some embodiments, the Linker-Drug compounds are designed for connection to a PBRM.

In some embodiments, the Drug Linker is

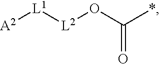

wherein the asterisk indicates the point of attachment to the Drug moiety (D, as defined above), A² is a Stretcher group (A′) to form a connection to a PBRM moiety, L¹ is a Specificity unit, L² (a Spacer unit) is a covalent bond or together with —OC(═O)— forms a self-immolative group(s).

In another embodiment, the Drug Linker compound is

A²-L¹-L²-

wherein the asterisk indicates the point of attachment to the Drug moiety (D), A² is a Stretcher unit (A1) to form a connection to a PBRM moiety, L¹ is a Specificity unit, L² (a Spacer unit) is a covalent bond or a self-immolative group(s). L¹ and L² are as defined above. References to connection to A¹ can be construed here as referring to a connection to A².

In some embodiments, where L¹ comprises an amino acid, the side chain of that amino acid may be protected. Any suitable protecting group may be used. In some embodiments, the side chain protecting groups are removable with other protecting groups in the compound, where present. In other embodiments, the protecting groups may be orthogonal to other protecting groups in the molecule, where present.

Suitable protecting groups for amino acid side chains include those groups described in the Novabiochem Catalog 2006/2007. Protecting groups for use in a cathepsin labile linker are also discussed in Dubowchik et al.

In certain embodiments, the group L¹ includes a Lys amino acid residue. The side chain of this amino acid may be protected with a Boc or Alloc protected group. A Boc protecting group is most preferred.

The functional group A² forms a connecting group upon reaction with a PBRM moiety.

In some embodiments, the functional group A² is or comprises an amino, carboxylic acid, hydroxy, thiol, or maleimide group for reaction with an appropriate group on the PBRM moiety. In a preferred embodiment, A² comprises a maleimide group.

In some embodiments, the group A² is an alkyl maleimide group. This group is suitable for reaction with thiol groups, particularly cysteine thiol groups, present in the PBRM, for example present in an antibody.

In some embodiments, the group A² is:

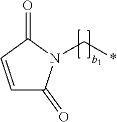

wherein the asterisk indicates the point of attachment to L¹, L² or D, and b₁ is an integer from 0 to 6. In some embodiments, b₁ is 5.

In some embodiments, the group A² is:

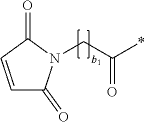

wherein the asterisk indicates the point of attachment to L¹, L² or D, and b₁ is an integer from 0 to 6. In some embodiments, b₁ is 5.

In some embodiments, the group A² is:

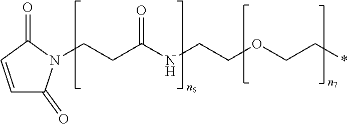

wherein the asterisk indicates the point of attachment to L, n₆ is an integer 0 or 1, and n₇ is an integer from 0 to 30. In a preferred embodiment, n₆ is 1 and n₇ is 0 to 10, 1 to 2, preferably 4 to 8, and most preferably 4 or 8.

In some embodiments, the group A² is:

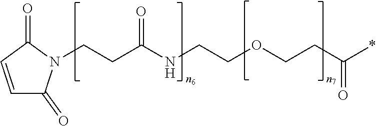

wherein the asterisk indicates the point of attachment to L, n₆ is an integer 0 or 1, and n₇ is an integer from 0 to 30. In a preferred embodiment, n₆ is 1 and n is 0 to 10, 1 to 8, preferably 4 to 8, and most preferably 4 or 8.

In some embodiments, the group A²:

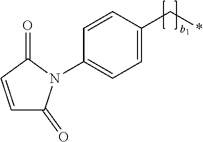

wherein the asterisk indicates the point of attachment to L¹, L² or D, and b₁ is an integer from 0 to 6. In some embodiments, b₁ is 5.

In some embodiments, the group A² is:

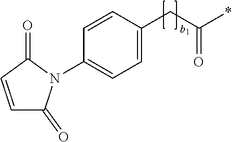

wherein the asterisk indicates the point of attachment to L₁, L² or D, and b₁ is an integer from 0 to 6. In some embodiments, b₁ is 5.

In some embodiments, the group A² is:

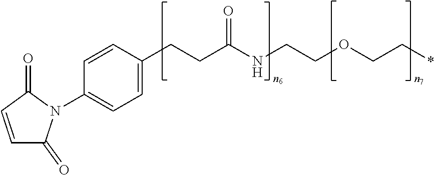

wherein the asterisk indicates the point of attachment to L¹, n₆ is an integer 0 or 1, and n₇ is an integer from 0 to 30. In a preferred embodiment, n₆ is 1 and n₇ is 0 to 10, 1 to 2, preferably 4 to 8, and most preferably 4 or 8.

In some embodiments, the group A² is:

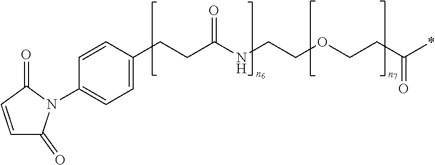

wherein the asterisk indicates the point of attachment to L₁, n₆ is an integer 0 or 1, and n₇ is an integer from 0 to 30. In a preferred embodiment, n₆ is 1 and n₇ is 0 to 10, 1 to 8, preferably 4 to 8, and most preferably 4 or 8.

In each of the embodiments above, an alternative functionality may be used in place of the malemide group shown below:

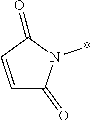 [SMILES: *N1C(=O)C=CC1=O]

wherein the asterisk indicates the bond to the remaining portion of the A² group.

In some embodiments, the maleimide-derived group is replaced with the group:

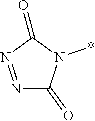 [SMILES: *N1C(=O)N=NC1=O]

wherein the asterisk indicates the bond to the remaining portion of the A² group.

In some embodiments, the maleimide group is replaced with a group selected from: (i) —C(═O)OH; (ii) —OH; (iii) —NH₂; (iv) —SH; (v) —C(═O)CH₂X₇; wherein X₇ is CI, Br or I; (vi) —CHO; (vii) —C═CH; and (viii) —N₃ (azide). Of these, —C(═O)CH₂X₇ may be preferred, especially when the carbonyl group is bound to —NH—.

In some embodiments, L¹ is present, and A² is —NH₂, —NHMe, —COOH, —OH or —SH.

In some embodiments, where L¹ is present, A² is —NH₂ or —NHMe. Either group may be the N-terminal of an L¹ amino acid sequence.

In some embodiments, L¹ is present and A² is —NH₂, and L¹ is an amino acid sequence —X₅—X₆—, as defined above.

In some embodiments, L¹ is present and A² is COOH. This group may be the C-terminal of an L¹ amino acid sequence.

In some embodiments, L¹ is present and A² is OH.

In some embodiments, L¹ is present and A² is SH.

The group A² may be convertible from one functional group to another. In one embodiment, L¹ is present and A² is —NH. This group is convertible to another group A² comprising a maleimide group. In some embodiments, the group —NH₂ may be reacted with an acids or an activated acid (e.g., N-succinimide forms) of those A² groups comprising maleimide shown above.

The group A² may therefore be converted to a functional group that is more appropriate for reaction with a PBRM moiety.

As noted above, In some embodiments, L¹ is present and A² is —NH₂, —NHMe, —COOH, —OH or —SH. In a further embodiment, these groups are provided in a chemically protected form. The chemically protected form is therefore a precursor to the linker that is provided with a functional group.

In some embodiments, A² is —NH₂ in a chemically protected form. The group may be protected with a carbamate protecting group. The carbamate protecting group may be selected from the group consisting of: Alloc, Fmoc, Boc, Troc, Teoc, Cbz and PNZ.

Preferably, where A² is —NH₂, it is protected with an Alloc or Fmoc group.

In some embodiments, where A² is —NH₂, it is protected with an Fmoc group.

In some embodiments, the protecting group is the same as the carbamate protecting group of the capping group.

In some embodiments, the protecting group is not the same as the carbamate protecting group of the capping group. In this embodiment, it is preferred that the protecting group is removable under conditions that do not remove the carbamate protecting group of the capping group.

The chemical protecting group may be removed to provide a functional group to form a connection to a PBRM moiety. Optionally, this functional group may then be converted to another functional group as described above.

In some embodiments, the active group is an amine. This amine is preferably the N-terminal amine of a peptide, and may be the N-terminal amine of the preferred dipeptides of the present disclosure. The active group may be reacted to yield the functional group that is intended to form a connection to a PBRM moiety.

In other embodiments, the Linker unit is a precursor to the Linker unit having an active group. In this embodiment, the Linker unit comprises the active group, which is protected by way of a protecting group. The protecting group may be removed to provide the Linker unit having an active group.

Where the active group is an amine, the protecting group may be an amine protecting group, such as those described in Green and Wuts. The protecting group is preferably orthogonal to other protecting groups, where present, the Linker unit.

In some embodiments, the protecting group is orthogonal to the capping group. Thus, the active group protecting group is removable whilst retaining the capping group. In other embodiments, the protecting group and the capping group is removable under the same conditions as those used to remove the capping group.

In some embodiments, the Linker unit is:

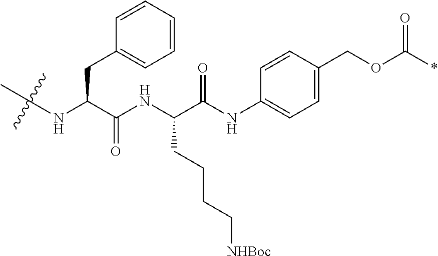 [SMILES: *N[C@@H](Cc1ccccc1)C(=O)N[C@@H](CCCCNC(=O)OC(C)(C)C)C(=O)Nc1ccc(COC(*)=O)cc1]

wherein the asterisk indicates the point of attachment to the Drug moiety, and the wavy line indicates the point of attachment to the remaining portion of the Linker unit, as applicable or the point of attachment to A². Preferably, the wavy line indicates the point of attachment to A².

In some embodiments, the Linker unit is:

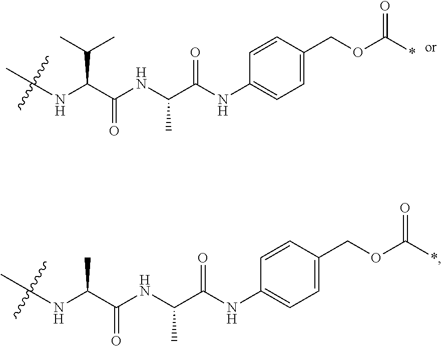

wherein the asterisk and the wavy line are as defined above.

Other functional groups suitable for use in forming a connection between L and the PBRM are described in WO 2005/082023.

### Protein-Based Recognition Molecules (PBRMs)

The protein-based recognition molecule directs the conjugates comprising a peptide linker to specific tissues, cells, or locations in a cell. The protein-based recognition molecule can direct the conjugate in culture or in a whole organism, or both. In each case, the protein-based recognition molecule has a ligand that is present on the cell surface of the targeted cell(s) to which it binds with an effective specificity, affinity and avidity. In some embodiments, the protein-based recognition molecule targets the conjugate to tissues other than the liver. In other embodiments the protein-based recognition molecule targets the conjugate to a specific tissue such as the liver, kidney, lung or pancreas. The protein-based recognition molecule can target the conjugate to a target cell such as a cancer cell, such as a receptor expressed on a cell such as a cancer cell, a matrix tissue, or a protein associated with cancer such as tumor antigen. Alternatively, cells comprising the tumor vasculature may be targeted. Protein-based recognition molecules can direct the conjugate to specific types of cells such as specific targeting to hepatocytes in the liver as opposed to Kupffer cells. In other cases, protein-based recognition molecules can direct the conjugate to cells of the reticular endothelial or lymphatic system, or to professional phagocytic cells such as macrophages or eosinophils. (In such cases the conjugate itself might also be an effective delivery system, without the need for specific targeting).

In still other embodiments, the protein based recognition molecule can target the conjugate to a location within the cell, such as the nucleus, the cytoplasm, or the endosome, for example. In specific embodiments, the protein based recognition molecule can enhance cellular binding to receptors, or cytoplasmic transport to the nucleus and nuclear entry or release from endosomes or other intracellular vesicles.

In specific embodiments the protein based recognition molecules include antibodies, proteins and peptides or peptide mimics.

In a preferred embodiment, the protein based recognition molecule comprises a sulfhydryl group and the protein based recognition molecule is conjugated to the Linker-Drug moiety by forming a covalent bond via the sulfhydryl group and a functional group of the Linker-Drug moiety.

Exemplary antibodies or antibodies derived from Fab, Fab2, scFv or camel antibody heavy-chain fragments specific to the cell surface markers, include, but are not limited to, 5T4, AOC3, ALK, AXL, C242, C4.4a, CA-125, CCL11, CCR 5, CD2, CD3, CD4, CD5, CD15, CA15-3, CD18, CD19, CA19-9, CDH6, CD20, CD22, CD23, CD25, CD28, CD30, CD31, CD33, CD37, CD38, CD40, CD41, CD44, CD44 v6, CD51, CD52, CD54, CD56, CD62E, CD62P, CD62L, CD70, CD74, CD79-B, CD80, CD125, CD138, CD141, CD147, CD152, CD 154, CD326, CEA, CEACAM-5, clumping factor, CTLA-4, CXCR2, EGFR (HER1), ErbB2, ErbB3, EpCAM, EPHA2, EPHB2, EPHB4, FGFR (i.e. FGFR1, FGFR2, FGFR3, FGFR4), FLT3, folate receptor, FAP, GD2, GD3, GPNMB, GCC (GUCY2C), HGF, HER2, HER3, HMI.24, ICAM, ICOS-L, IGF-1 receptor, VEGFR1, EphA2, TRPV1, CFTR, gpNMB, CA9, Cripto, c-KIT, c-MET, ACE, APP, adrenergic receptor-beta2, Claudine 3, LIV, LY6E, Mesothelin, MUC1, MUC13, NaPi2b, NOTCH1, NOTCH2, NOTCH3, NOTCH4, RON, ROR1, PD-L1, PD-L2, PTK7, B7-H3, B7-B4, IL-2 receptor, IL-4 receptor, IL-13 receptor, TROP-2, frizzled-7, integrins (including α₄, αᵥβ₃, αᵥβ₅, αᵥβ₆, αᵥβ₄, α₄β₁, α₄β₇, α₅β₁, α₅β₁, α₆β₄, α_{IIb}β₃ intergins), IFN-α, IFN-γ, IgE, IgE, IGF-1 receptor, IL-1, IL-12, IL-23, IL-13, IL-22, IL-4, IL-5, IL-6, interferon receptor, ITGB2 (CD18), LFA-1 (CD11a), L-selectin (CD62L), mucin, myostatin, NCA-90, NGF, PDGFRα, phosphatidylserine, prostatic carcinoma cell, Pseudomonas aeruginosa, rabies, RANKL, respiratory syncytial virus, Rhesus factor, SLAMF7, sphingosine-1-phosphate, TAG-72, T-cell receptor, tenascin C, TGF-1, TGF-β2, TGF-β, TNF-α, TRAIL-R1, TRAIL-R2, tumor antigen CTAA16.88, VEGF-A, VEGFR2, vimentin, and the like.

In some embodiments, the antibodies or antibody derived from Fab, Fab2, scFv or camel antibody heavy-chain fragments specific to the cell surface markers include CA-125, C242, CD3, CD19, CD22, CD25, CD30, CD31, CD33, CD37, CD40, CD44, CD51, CD54, CD56, CD62E, CD62P, CD62L, CD70, CD138, CD141, CD326, CEA, CTLA-4, EGFR (HER1), ErbB2, ErbB3, FAP, folate receptor, IGF-1 receptor, GD3, GPNMB, HGF, HER2, VEGF-A, VEGFR2, VEGFR1, EphA2, EpCAM, 5T4, TAG-72, tenascin C, TRPV1, CFTR, gpNMB, CA9, Cripto, ACE, APP, PDGFR α, phosphatidylserine, prostatic carcinoma cells, adrenergic receptor-beta2, Claudine 3, mucin, MUC1, NaPi2b, B7H3, B7H4, C4.4a, CEACAM-5, MUC13, TROP-2, frizzled-7, Mesothelin, IL-2 receptor, IL-4 receptor, IL-13 receptor and integrins (including αᵥβ₃, αᵥβ₅, αᵥβ₆, α₁β₄, α₄β₁, α₅β₁, α₆β₄ intergins), tenascin C, TRAIL-R₂ and vimentin.

Exemplary antibodies include 3F8, abagovomab, abciximab (REOPRO), adalimumab (HUMIRA), adecatumumab, afelimomab, afutuzumab, alacizumab, ALD 518, alemtuzumab (CAMPATH), altumomab, amatuximab, anatumomab, anrukinzumab, apolizumab, arcitumomab (CEA-SCAN), aselizumab, atlizumab (tocilizumab, Actemra, RoActemra), atorolimumab, bapineuzumab, basiliximab (Simulect), bavituximab, bectumomab (LYMPHOSCAN), belimumab (BENLYSTA), benralizumab, bertilimumab, besilesomab (SCINITIMUN), bevacizumab (AVASTIN), biciromab (FIBRISCINT), bivatuzumab, blinatumomab, brentuximab, briakinumab, canakinumab (ILARIS), cantuzumab, capromab, catumaxomab (REMOVAB), CC49, cedelizumab, certolizumab, cetuximab (ERBITUX), citatuzumab, cixutumumab, clenoliximab, clivatuzumab, conatumumab, CR6261, dacetuzumab, daclizumab (ZENAPAX), daratumumab, denosumab (PROLIA), detumomab, dorlimomab, dorlixizumab, ecromeximab, eculizumab (SOLRIS), edobacomab, edrecolomab (PANOREX), efalizumab (RAPTIVA), efungumab (MYCOGRAB), elotuzumab, elsilimomab, enlimomab, epitumomab, epratuzumab, erlizumab, ertumaxomab (REXOMUN), etaracizumab (ABEGRIN), exbivirumab, fanolesomab (NEUTROSPEC), faralimomab, farletuzumab, felvizumab, fezakinumab, figitumumab, fontolizumab (HuZAF), foravirumab, fresolimumab, galiximab, gantenerumab, gavilimomab, gemtuzumab, girentuximab, glembatumumab, goimumab (SIMPONI), gomiliximab, ibalizumab, ibritumomab, igovomab (INDIMACIS-125), imciromab (MYOSCINT), infliximab (REMICADE), intetumumab, inolimomab, inotuzumab, ipilimumab, iratumumab, keliximab, labetuzumab (CEA-CIDE), lebrikizumab, lemalesomab, lerdelimumab, lexatumumab, libivirumab, lintuzumab, lucatumumab, lumiliximab, mapatumumab, maslimomab, matuzumab, mepolizumab (BOSATRIA), metelimumab, milatuzumab, minretumomab, mitumomab, morolimumab, motavizumab (NUMAX), muromonab-CD3 (ORTHOCLONE OKT3), nacolomab, naptumomab, natalizumab (TYSABRI), nebacumab, necitumumab, nerelimomab, nimotuzumab (THERACIM), nofetumomab, ocrelizumab, odulimomab, ofatumumab (ARZERRA), olaratumab, omalizumab (XOLAIR), ontecizumab, oportuzumab, oregovomab (OVAREX), otelixizumab, pagibaximab, palivizumab (SYNAGIS), panitumumab (VECTIBIX), panobacumab, pascolizumab, pemtumomab (THERAGYN), pertuzumab (OMNITARG), pexelizumab, pintumomab, priliximab, pritumumab, PRO 140, rafivirumab, ramucirumab, ranibizumab (LUCENTIS), raxibacumab, regavirumab, reslizumab, rilotumumab, rituximab (RITUXAN), robatumumab, rontalizumab, rovelizumab (LEUKARREST), ruplizumab (ANTOVA), satumomab pendetide, sevirumab, sibrotuzumab, sifalimumab, siltuximab, siplizumab, solanezumab, sonepcizumab, sontuzumab, stamulumab, sulesomab (LEUKOSCAN), tacatuzumab (AFP-CIDE), tetraxetan, tadocizumab, talizumab, tanezumab, taplitumomab paptox, tefibazumab (AUREXIS), telimomab, tenatumomab, teneliximab, teplizumab, TGN1412, ticilimumab (tremelimumab), tigatuzumab, TNX-650, tocilizumab (atlizumab, ACTEMRA), toralizumab, tositumomab (BEXXAR), trastuzumab (HERCEPTIN), tremelimumab, tucotuzumab, tuvirumab, urtoxazumab, ustekinumab (STELERA), vapaliximab, vedolizumab, veltuzumab, vepalimomab, visilizumab (NUVION), volociximab (HUMASPECT), votumumab, zalutumumab (HuMEX-EGFr), zanolimumab (HuMAX-CD4), ziralimumab and zolimomab.

In some embodiments, the antibodies are directed to cell surface markers for 5T4, CA-125, CEA, CDH6, CD3, CD19, CD20, CD22, CD30, CD33, CD40, CD44, CD51, CTLA-4, CEACAM5, EpCAM, HER2, EGFR (HER1), FAP, folate receptor, GCC (GUCY2C), HGF, integrin αᵥβ₃, integrin α₅β₁, IGF-1 receptor, GD3, GPNMB, mucin, LIV1, LY6E, mesothelin, MUC1, MUC13, PTK7, phosphatidylserine, prostatic carcinoma cells, PDGFRα, TAG-72, tenascin C, TRAIL-R2, VEGF-A and VEGFR2. In this embodiment the antibodies are abagovomab, adecatumumab, alacizumab, altumomab, anatumomab, arcitumomab, bavituximab, bevacizumab (AVASTIN), bivatuzumab, blinatumomab, brentuximab, cantuzumab, catumaxomab, capromab, cetuximab, citatuzumab, clivatuzumab, conatumumab, dacetuzumab, edrecolomab, epratuzumab, ertumaxomab, etaracizumab, farletuzumab, figitumumab, gemtuzumab, glembatumumab, ibritumomab, igovomab, intetumumab, inotuzumab, labetuzumab, lexatumumab, lintuzumab, lucatumumab, matuzumab, mitumomab, naptumomab estafenatox, necitumumab, oportuzumab, oregovomab, panitumumab, pemtumomab, pertuzumab, pritumumab, rituximab (RITUXAN), rilotumumab, robatumumab, satumomab, sibrotuzumab, taplitumomab, tenatumomab, tenatumomab, ticilimumab (tremelimumab), tigatuzumab, trastuzumab (HERCEPTIN), tositumomab, tremelimumab, tucotuzumab celmoleukin, volociximab and zalutumumab.

In specific embodiments the antibodies directed to cell surface markers for HER2 are pertuzumab or trastuzumab and for EGFR (HER1) the antibody is cetuximab or panitumumab; and for CD20 the antibody is rituximab and for VEGF-A is bevacizumab and for CD-22 the antibody is epratuzumab or veltuzumab and for CEA the antibody is labetuzumab.

Exemplary peptides or peptide mimics include integrin targeting peptides (RGD peptides), LHRH receptor targeting peptides, ErbB2 (HER2) receptor targeting peptides, prostate specific membrane bound antigen (PSMA) targeting peptides, lipoprotein receptor LRP1 targeting, ApoE protein derived peptides, ApoA protein peptides, somatostatin receptor targeting peptides, chlorotoxin derived peptides, and bombesin.

In specific embodiments the peptides or peptide mimics are LHRH receptor targeting peptides and ErbB2 (HER2) receptor targeting peptides.

Exemplary proteins comprise insulin, transferrin, fibrinogen-gamma fragment, thrombospondin, claudin, apolipoprotein E, Affibody molecules such as, for example, ABY-025, Ankyrin repeat proteins, ankyrin-like repeats proteins and synthetic peptides.

In some embodiments, the protein-drug conjugates comprise broad spectrum cytotoxins in combination with cell surface markers for HER2 such as pertuzumab or trastuzumab; for EGFR such as cetuximab and panitumumab; for CEA such as labetuzumab; for CD20 such as rituximab; for VEGF-A such as bevacizumab; or for CD-22 such as epratuzumab or veltuzumab.

In other embodiments, the protein-drug conjugates or protein conjugates used in the disclosure comprise combinations of two or more protein based recognition molecules, such as, for example, combination of bispecific antibodies directed to the EGF receptor (EGFR) on tumor cells and to CD3 and CD28 on T cells; combination of antibodies or antibody derived from Fab, Fab2, scFv or camel antibody heavy-chain fragments and peptides or peptide mimetics; combination of antibodies or antibody derived from Fab, Fab2, scFv or camel antibody heavy-chain fragments and proteins; combination of two bispecific antibodies such as CD3×CD19 plus CD28×CD22 bispecific antibodies.

In other embodiments, the protein-drug conjugates or protein conjugates used in the disclosure comprise protein based recognition molecules are antibodies against antigens, such as, for example, Trastuzumab, Cetuximab, Rituximab, Bevacizumab, Epratuzumab, Veltuzumab, Labetuzumab, B7-H4, B7-H3, CA125, CDH6, CD33, CXCR2, CEACAM5, EGFR, FGFR1, FGFR2, FGFR3, FGFR4, GCC (GUCY2C), HER2, LIV1, LY6E, NaPi2b, c-Met, mesothelin, NOTCH1, NOTCH2, NOTCH3, NOTCH4, PD-L1, PTK7, c-Kit, MUC1, MUC13. and 5T4.

In a specific embodiment, the protein-drug conjugates or protein conjugates of the disclosure comprise protein based recognition molecules which are antibodies against 5T4, such as, for example a humanized anti-5T4 scFvFc antibody.

Examples of suitable 5T4 targeting ligands or immunoglobulins include those which are commercially available, or have been described in the patent or non-patent literature, e.g., U.S. Pat. Nos. 8,044,178, 8,309,094, 7,514,546, EP1036091 (commercially available as TroVax™, Oxford Biomedica), EP2368914A1, WO 2013041687 A1 (Amgen), US 2010/0173382, and P. Sapra, et al., Mol. Cancer Ther. 2013, 12:38-47. An anti-5T4 antibody is disclosed in U.S. Provisional Application No. 61/877,439, filed Sep. 13, 2013 and U.S. Provisional Application No. 61/835,858, filed Jun. 17, 2013. The contents of each of the patent documents and scientific publications are herein incorporated by reference in their entireties.

As used herein, the term “5T4 antigen-binding portion” refers to a polypeptide sequence capable of selectively binding to a 5T4 antigen. In exemplary conjugates, the 5T4 antigen-binding portion generally comprises a single chain scFv-Fc form engineered from an anti-5T4 antibody. A single-chain variable fragment (scFv-Fc) is a fusion protein of the variable regions of the heavy (VH) and light chains (VL) of an immunoglobulin, connected with a linker peptide, and further connected to an Fc region comprising a hinge region and CH2 and CH3 regions of an antibody (any such combinations of antibody portions with each other or with other peptide sequences is sometimes referred to herein as an “immunofusion” molecule). Within such a scFvFc molecule, the scFv section may be C-terminally linked to the N-terminus of the Fc section by a linker peptide.

In other specific embodiments, the protein-drug conjugates or protein conjugates of the disclosure comprise protein based recognition molecules which are Her-2 or NaPi2b antibodies.

In some embodiments, the Her-2 antibody suitable for the conjugate or scaffold of the disclosure comprises a variable heavy chain complementarity determining region 1 (CDRH1) comprising the amino acid sequence FTFSSYSMN (SEQ ID NO: 1); a variable heavy chain complementarity determining region 2 (CDRH2) comprising the amino acid sequence YISSSSSTIYYADSVKG (SEQ ID NO: 2); a variable heavy chain complementarity determining region 3 (CDRH3) comprising the amino acid sequence GGHGYFDL (SEQ ID NO: 3); a variable light chain complementarity determining region 1 (CDRL1) comprising the amino acid sequence RASQSVSSSYLA (SEQ ID NO: 4); a variable light chain complementarity determining region 2 (CDRL2) comprising the amino acid sequence GASSRAT (SEQ ID NO: 5); and a variable light chain complementarity determining region 3 (CDRL3) comprising the amino acid sequence QQYHHSPLT (SEQ ID NO: 6) (see, e.g., US20150366987(A1) published Dec. 24 2015).

In some embodiments, the NaPi2b antibody suitable for the conjugate or scaffold of the disclosure comprises a variable light chain complementarity determining region 1 (CDRL1) comprising the amino acid sequence SASQDIGNFLN (SEQ ID NO: 7); a variable light chain complementarity determining region 2 (CDRL2) comprising the amino acid sequence YTSSLYS (SEQ ID NO: 8); a variable light chain complementarity determining region 3 (CDRL3) comprising the amino acid sequence QQYSKLPLT (SEQ ID NO: 9); a variable heavy chain complementarity determining region 1 (CDRH1) comprising the amino acid sequence GYTFTGYNIH (SEQ ID NO: 10); a variable heavy chain complementarity determining region 2 (CDRH2) comprising the amino acid sequence AIYPGNGDTSYKQKFRG (SEQ ID NO: 11); and a variable heavy chain complementarity determining region 3 (CDRH3) comprising the amino acid sequence GETARATFAY (SEQ ID NO: 12) (see, e.g., co-pending application U.S. Ser. No. 15/457,574 filed Mar. 13, 2017).

### PBD Drug Moiety (D)

In some embodiments, the PBD drug moiety (D) is of Formula (IV),

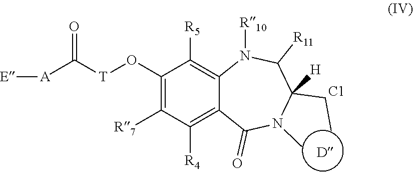

a tautomer thereof; a pharmaceutically acceptable salt or solvate thereof, or a pharmaceutically acceptable salt or solvate of the tautomer, wherein:

E″ is a direct or indirect linkage to the PBRM (e.g., antibody or antibody fragment), or

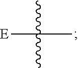

in which

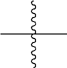

denotes direct or indirect linkage to the PBRM (e.g., antibody or antibody fragment) via a functional group of E;

D″ is D′ or

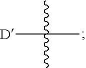

in which

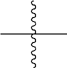

denotes direct or indirect linkage to the PBRM (e.g., antibody or antibody fragment) via a functional group of D′;

R″₇ is a direct or indirect linkage to the PBRM (e.g., antibody or antibody fragment), R₇, or

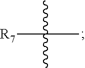

in which

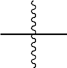

denotes direct or indirect linkage to the PBRM (e.g., antibody or antibody fragment) via a functional group of R₇;

R″₁₀ is a direct or indirect linkage to the PBRM (e.g., antibody or antibody fragment), R₁₀, or

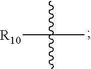

in which

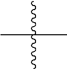

denotes direct or indirect linkage to the PBRM (e.g., antibody or antibody fragment) via a functional group of R₁₀; and

wherein the PBD drug moiety (D) is directly or indirectly linked to the PBRM (e.g., antibody or antibody fragment) via a functional group of one of E″, D″, R″₇, and R″₁₀.

In some embodiments, E″ is a direct or indirect linkage to L^{C}, E, or

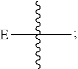

in which

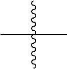

denotes direct or indirect linkage to L^{C} via a functional group of E.

In some embodiments, E″ is a direct or indirect linkage to L^{D}, E, or

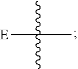

in which

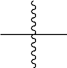

denotes direct or indirect linkage to L^{D} via a functional group of E.

In some embodiments, D″ is D′ or

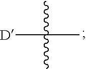

in which

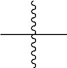

denotes direct or indirect linkage to L^{C} via a functional group of D′.

In some embodiments, D″ is D′ or

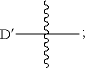

in which

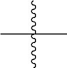

denotes direct or indirect linkage to L^{D} via a functional group of D′.

In some embodiments, R″₇ is a direct or indirect linkage to L^{C}, R₇ or

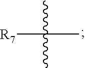

in which

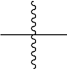

denotes direct or indirect linkage to L^{C} via a functional group of R₇.

In some embodiments, R″₇ is a direct or indirect linkage to L^{D}, R₇ or

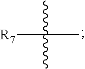

in which

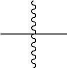

denotes direct or indirect linkage to L^{D} via a functional group of R₇.

In some embodiments, R″₁₀ is a direct or indirect linkage to L^{C}, R₁₀, or

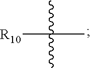

in which

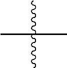

denotes direct or indirect linkage L^{C} via a functional group of R₁₀.

In some embodiments, R″₁₀ is a direct or indirect linkage to L^{D}, R₁₀, or

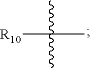

in which

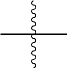

denotes direct or indirect linkage L^{C} via a functional group of R₁₀.

In some embodiments, E″ is a direct or indirect linkage to the PBRM; D″ is D′; R″₇ is R₇ and R″₁₀ is R₁₀.

In some embodiments, E″ is a direct or indirect linkage to L^{C}; D″ is D′; R″₇ is R₇ and R″₁₀ is R₁₀.

In some embodiments; E″ is a direct or indirect linkage to L^{D}; D″ is D′; R″₇ is R₇ and

In some embodiments, E″ is

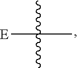

in which

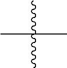

denotes direct or indirect linkage to the PBRM via a functional group of E; D″ is D′; R″₇ is R₇; and R″₁₀ is R₁₀.

In some embodiments, E″ is

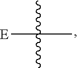

in which

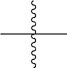

denotes direct or indirect linkage to L^{C} via a functional group of E; D″ is D′; R″₇ is R₇; and R″₁₀ is R₁₀.

In some embodiments; E″ is

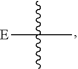

in which

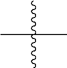

denotes direct or indirect linkage to L^{D} via a functional group of E; D″ is D′; R″₇ is R₇; and R″₁₀ is R₁₀.

In some embodiments, D″ is

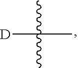 [SMILES: *[2H]]

in which

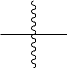

denotes direct or indirect linkage to the PBRM via a functional group of D; E″ is E; R″₇ is R₇; and R″₁₀ is R₁₀.

In some embodiments, D″ is

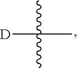 [SMILES: *[2H]]

in which

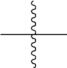

denotes direct or indirect linkage to L^{C} via a functional group of D; E″ is E; R″₇ is R₇; and R″₁₀ is R₁₀.

In some embodiments, D″ is

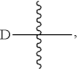 [SMILES: *[2H]]

in which

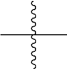

denotes direct or indirect linkage to L^{D} via a functional group of D; E″ is E; R″₇ is R₇; and R″₁₀ is R₁₀.

In some embodiments, R″₇ is a direct or indirect linkage to the PBRM; E″ is E; D″ is D′; and R″₁₀ is R₁₀.

In some embodiments, R″₇ is a direct or indirect linkage to L^{C}; E″ is E; D″ is D′; and R″₁₀ is R₁₀.

In some embodiments, R″₇ is a direct or indirect linkage to L^{u}; E″ is E; D″ is D′; and R″₁₀ is R₁₀.

In some embodiments, R″₇ is

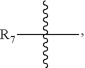

in which

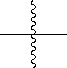

denotes direct or indirect linkage to the PBRM via a functional group of R₇; E″ is E; D″ is D′; and R″₁₀ is R₁₀.

In some embodiments, R″₇ is

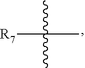

in which

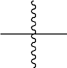

denotes direct or indirect linkage to L^{C} via a functional group of R₇; E″ is E; D″ is D′; and R″₁₀ is R₁₀.

In some embodiments; R″₇ is

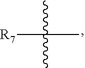

in which

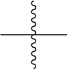

denotes direct or indirect linkage to L^{D} via a functional group of R₇; E″ is E; D″ is D′; and R″₁₀ is R₁₀.

In some embodiments, R″₁₀ is a direct or indirect linkage to the PBRM; E″ is E; D″ is D′; and R″₇ is R₇.

In some embodiments, R″₁₀ is a direct or indirect linkage to L^{C}; E″ is E; D″ is D′; and R″₇ is R₇.

In some embodiments, R″₁₀ is a direct or indirect linkage to L^{D}; E″ is F; D″ is D′; and R″₇ is R₇.

In some embodiments, R″₁₀ is

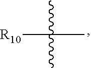

in which

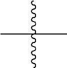

denotes direct or indirect linkage to the PBRM via a functional group of R₁₀; E″ is E; D″ is D′; and R″₇ is R₇.

In some embodiments, R″₁₀ is

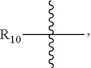

in which

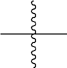

denotes direct or indirect linkage to L^{C} via a functional group of R₁₀; E″ is E; D″ is D′; and R″₇ is R₇.

In some embodiments, R″₁₀ is

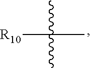

in which

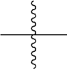

denotes direct or indirect linkage to L^{D} via a functional group of R₁₀; E″ is E; D″ is D′; and R″₇ is R₇.

In some embodiments, the conjugates of Formula (IV) include those where each of the moieties defined for one of E″, D″, R″₇, R″₁₀, D′, T, E, A, R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₃₁, R₃₂, R₃₃, R₃₄, R₃₅ₐ, B_{35b}, R₃₆ₐ, R_{36b}, R_{36c}, R_{36d}, R₃₇ₐ, R_{37b}, Rₐ, R^{b}, R^{N}, R^{Q}, X₀, Y₀, Z₀, X₁, Y₁, Z₁, X₂, X₃, X₄, X₈, M, Q, m, n, r, s, t, and x, can be combined with any of the moieties defined for the others of E″, D″, R″₇, R″₁₀, D′, T, E, A, R₂, R₃, R₄, R₅, R₆, R₇, R₈, R₉, R₁₀, R₁₁, R₁₂, R₁₃, R₁₄, R₁₅, R₁₆, R₁₉, R₂₀, R₂₁, R₃₁, R₃₂, R₃₃, R₃₄, R₃₅ₐ, R_{35b}, R₃₆ₐ, R_{36b}, R_{36c}, R_{36d}, R₃₇ₐ, R_{37b}, R₄₀, Rₐ, R^{b}, R^{N}, R^{Q}, X₀, Y₀, Z₀, X₁, Y₁, Z₁, X₂, X₃, X₄, X₈, M, Q, m, n, r, s, t, and x.

In some embodiments, D′ is D1, D2, D3, or D4:

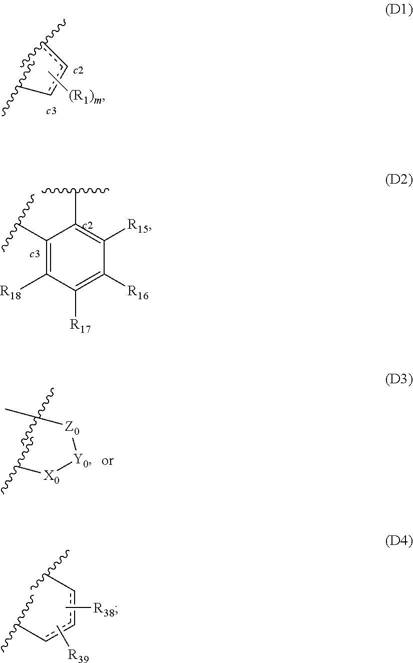

wherein the dotted line between C2 and C3 or between C2 and C1 in D1 or the dotted line in D4 indicates the presence of a single or double bond; and

m is 0, 1 or 2;

when D′ is D1, the dotted line between C2 and C3 is a double bond, and m is 1, then R₁ is:

(i) C6-10 aryl group, optionally substituted by one or more substituents selected from —OH, halo, —NO₂, —CN, —N₃, —OR₂, —COOH, —COOR₂, —COR, —OOONR₁₃R₁₄, alkyl, C3-10 cycloalkyl, C2-10 alkenyl, C2-10 alkynyl, a polyethylene glycol unit —(OCH₂CH₂)ᵣ—ORₐ, 3- to 14-membered heterocycloalkyl, 5- to 12-membered heteroaryl, bis-oxy-C₁₋₃ alkylene, —NR₁₃R₁₄, —S(═O)₂R₁₂, —S(═O)₂NR₁₃R₁₄, —SR₁₂, —SOₓM, —OSOₓM, —NR₉COR₁₉, —NH(C═NH)NH₂:

(ii) C₁₋₅ alkyl;

(iii) C₃₋₆ cycloalkyl;

(iv)

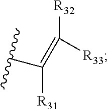

(vi)

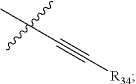

(vii)

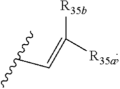

(viii)

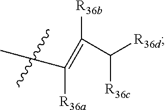

or

(viii) halo;

when D′ is D1, the dotted line between C₂ and C₃ is a single bond, and m is 1, then R₁ is:

(i) —OH, ═O, ═CH₂, —CN, —R₂, —OR₂, halo, ═CH—R₆, ═C(R₆)₂, —O—SO₂R₂, —CO₂R₂, —COR₂, —CHO, or —COOH; or

(ii)

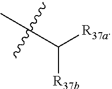

when D′ is D1 and m is 2, then each R₁ independently is halo and either both R₁ are attached to the same carbon atom or one is attached to C₂ and the other is attached to C₃;

T is C₁₋₁₀ alkylene linker;

A is

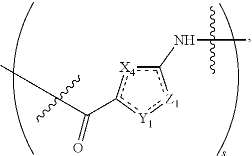

wherein the —NH group of A is connected to the —C(O)-T- moiety of Formula (IV) and the C═O moiety of A is connected to E; and each

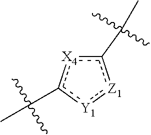

independently is

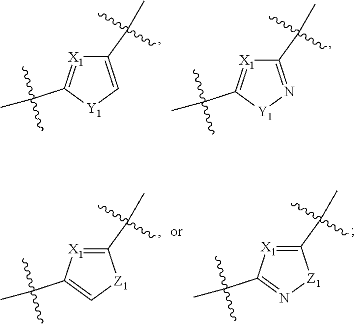

E is E1, E2, E3, E4, E5 or E6:

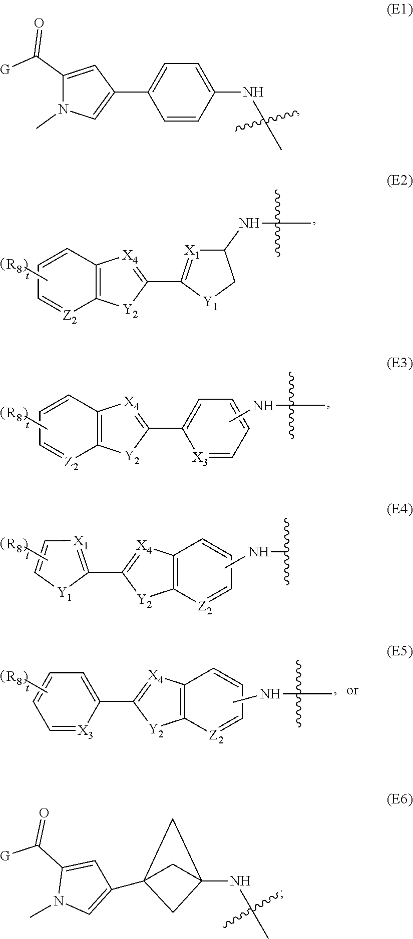

G is G1, G2, G3, G4, —OH, —NH—(C₁₋₆ alkylene)-R₁₃ₐ, —NR₁₃R₁₄, O—(CH₂)₃—NH₂, —O—CH(CH₃)—(CH₂)₂—NH₂, or —NH—(CH₂)₃—O—C(═O)—CH(CH₃)—NH₂:

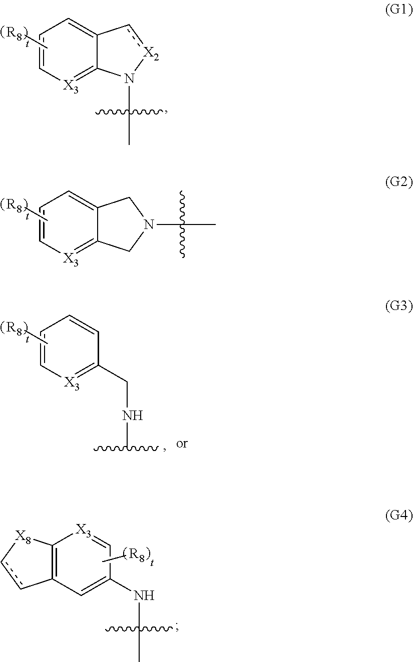

wherein the dotted line in G1 or G4 indicates the presence of a single or double bond;

each occurrence of R₂ and R₃ independently is an optionally substituted C₁₋₈ alkyl, optionally substituted C₂₋₈ alkenyl, optionally substituted C₂₋₈ alkynyl, optionally substituted C₃₋₈ cycloalkyl, optionally substituted 3- to 20-membered heterocycloalkyl, optionally substituted C₆₋₂₀ aryl or optionally substituted 5- to 20-membered heteroaryl, and, optionally in relation to the group NR₂R₃, R₂ and R₃ together with the nitrogen atom to which they are attached form an optionally substituted 4-, 5-, 6- or 7-membered heterocycloalkyl or an optionally substituted 5- or 6-membered heteroaryl;

R₄, R₅ and R₇ are each independently —H, —R₂, —OH, —OR₂, —SH, —SR₂, —NH₂, —NHR₂, —NR₂R₃, —NO₂, —SnMe₃, halo or a polyethylene glycol unit —(OCH₂CH₂)ᵣ—ORₐ; or R₄ and R₇ together form bis-oxy-C₁₋₃ alkylene;

each R₆ independently is —H, —R₂, —CO₂R₂, —COR₂, —CHO, —CO₂H, or halo;

each R₅ independently is —OH, halo, —NO₂, —CN, —N₃, —OR₂, —COOH, —COOR₂, —COR₂, —OCONR₁₃R₁₄, —CONR₁₃R₁₄, —CO—NH—(C₁₋₆ alkylene)-R₁₃ₐ, C₁₋₁₀ alkyl, C₃₋₁₀ cycloalkyl, C₂₋₁₀ alkenyl, C₂₋₁₀ alkynyl, a polyethylene glycol unit —(OCH₂CH₂)ᵣ—ORₐ, 3- to 14-membered heterocycloalkyl, 5- to 12-membered heteroaryl, —S(═O)₂R₁₂, —S(═O)₂NR₁₃R₁₄, —SR₁₂, —SOₓM, —OSOₓM, —NR₉COR₁₉, —NH(C═NH)NH₂, —R₂(—R₂₁—NR₁₃R₁₄, —R₂₀-R₂₁—NH—P(O)(OH)—(OCH₂CH₂)ₙ₉—OCH₃, or —O—P(O)(OH)—(OCH₂CH₂)ₙ₉—OCH₃;

each R₉ independently is C₁₋₁₀ alkyl, C₃₋₁₀ cycloalkyl, C₂₋₁₀ alkenyl or C₂₋₁₀ alkynyl;

R¹⁰ is —H or a nitrogen protecting group;

R¹¹ is -QR^{Q} or —SOₓM;

or R and R¹¹ taken together with the nitrogen atom and carbon atom to which they are respectively attached, form a N═C double bond;

each R₁₂ independently is C₁₋₇ alkyl, 3- to 20-membered heterocycloalkyl, 5- to 20-membered heteroaryl, or C₆₋₂₀ aryl;

each occurrence of R₁ and R₁₄ are each independently H, C₁₋₁₀ alkyl, 3- to 20-membered heterocycloalkyl, 5- to 20-membered heteroaryl, or C₆₋₂₀ aryl;

each R₁₃ₐ independently is —OH or —NR₁₃R₁₄;

R₁₅, R₁₆, R₁₇ and R₁₈ are each independently —H, —OH, halo, —NO₂, —CN, —N₃, —OR₂, —COOH, —COOR₂, —COR₂, —OCONR₁₃R₁₄, C₁₋₁₀ alkyl, C₃₋₁₀ cycloalkyl, C₂₋₁₀ alkenyl, C₂₋₁₀ alkynyl, a polyethylene glycol unit —(OCH₂CH₂)ᵣ—ORₐ, 3-14 membered heterocycloalkyl, 5- to 12-membered heteroaryl, —NR₁₃R₁₄, —S(═O)₂R₂, —S(═O)₂NR₁₃R₁₄, —SR₁₂, —SOₓM, —OSOₓM, —NR₉COR₁₉ or —NH(C═NH)NH₂;

each R₁₈ independently is C₁₋₁₀ alkyl, C₃₋₁₀ cycloalkyl, C₂₋₁₀ alkenyl or C₂₋₁₀ alkynyl;

each R₂₀ independently is a bond, C₆₋₁₀ arylene, 3-14 membered heterocycloalkylene or 5- to 12-membered heteroarylene;

each R₂₁ independently is a bond or C₁₋₁₀ alkylene;

R₃₁, R₃₂ and R₃₃ are each independently —H, C₁₋₃ alkyl, C₂₋₃ alkenyl, C₂₋₃ alkynyl or cyclopropyl, wherein the total number of carbon atoms in the R₁ group is no more than 5;

R₃₄ is —H, C₁₋₃ alkyl, C₂₋₃ alkenyl, C₂₋₃ alkynyl, cyclopropyl, or phenyl wherein the phenyl is optionally substituted by one or more of halo, methyl, methoxy, pyridyl or thiophenyl;

one of R₃₅ₐ and R_{35b} is —H and the other is a phenyl group optionally substituted with one or more of halo, methyl, methoxy, pyridyl or thiophenyl;

R₃₆ₐ, R_{36b}, R_{36c} are each independently —H or C₁₋₂ alkyl;

R_{36d} is —OH, —SH, —COOH, —C(O)H, —N═C═O, —NHNH₂, —CONHNH₂,

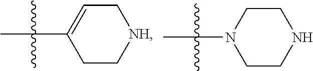 [SMILES: *C1=CCNCC1 ; *N1CCNCC1]

or NHR^{N}, wherein R^{N} is —H or C₁₋₄ alkyl;

R₃₇ₐ and R_{37b} are each independently is —H, —F, C₁₋₄ alkyl, C₂₋₃ alkenyl, wherein the alkyl and alkenyl groups are optionally substituted by C₁₋₄ alkyl amido or C₁₋₄ alkyl ester; or when one of R₃₇ₐ and R_{37b} is —H, the other is —CN or a C₁₋₄ alkyl ester;

R₃₈ and R₃₉ are each independently H, R₃, ═CH₂, ═CH—(CH₂)ₛ₁—CH₃, ═O, (CH₂)ₛ₁—OR₁₃, (CH₂)ₛ₁—CO₂R₁₃, (CH₂)ₛ₁—NR₁₃R₁₄, O—(CH₂)₂—NR₁₃R₁₄, NH—C(O)—R, O—(CH₂)ₛ₁—NH—C(O)—R₁₃, O—(CH₂)s-C(O)NHR₁₃, (CH₂)ₛ₁0S(═O)₂R₁₃, O—SO₂R₁₃, (CH₂)ₛ₁—C(O)R₁₃ and (CH₂)ₛ₁—C(O)NR₁₃R₁₄;

X₀ is CH₂, NR₆, C═O, BH, SO or SO₂;

Y₀ is O, CH₂, NR₆ or S;

Z₀ is absent or (CH₂)ₙ;

each X₁ independently is CR_{b}, or N;

each Y₁ independently is CH, NRₐ, O or S;

each Z₁ independently is CH, NRₐ, O or S;

each Rₐ independently is H or C₁₋₄ alkyl;

each R_{b} independently is H, OH, C₁₋₄ alkyl, or C₁₋₄ alkoxyl;

X₂ is CH, CH₂ or N;

X₃ is CH or N;

X₄ is NH, O or S;

X₈ is NH, O or S;

Q is O, S or NH;

when Q is S or NH, then R^{Q} is —H or optionally substituted C₁₋₂ alkyl; or

when Q is O, then R^{Q} is —H or optionally substituted C₁₋₂ alkyl, —SOₓM, —PO₃M, —(CH₂—CH₂—O)ₙ₉CH₃, —(CH₂—CH₂O)ₙ₉—(CH₂)₂—R₄₀, —C(O)—(CH₂—CH₂—O)ₙ₉CH₃, —C(O)O—(CH₂—CH₂—O)ₙ₉CH₃, —C(O)NH—(CH₂—CH₂—O)ₙ₉CH₃, —(CH₂)ₙ—NH—C(O)—CH₂—O—CH₂—C(O)—NH—(CH₂—CH₂—O)ₙ₉CH₃, —(CH₂)ₙ—NH—C(O)— (CH₂)ₙ—(CH₂—CH₂—O)ₙ₉CH₃, a sugar moiety,

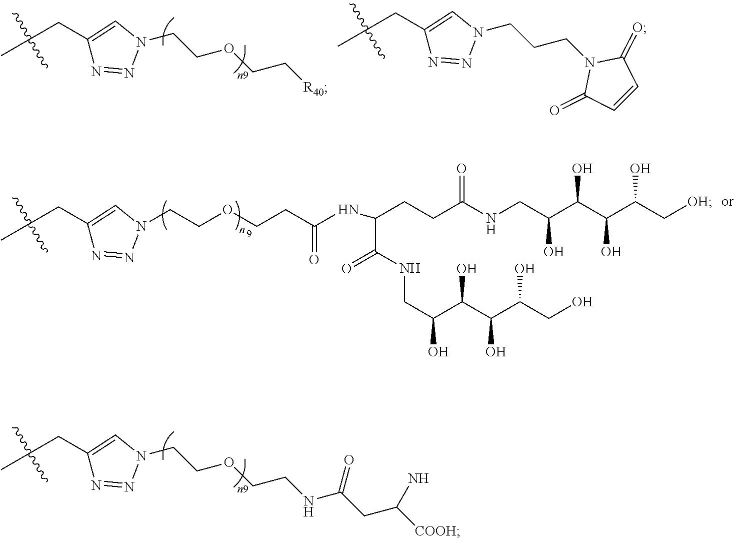

each M independently is H or a monovalent pharmaceutically acceptable cation;

n is 1, 2 or 3;

each r independently is an integer from 1 to 200;

s is 1, 2, 3, 4, 5 or 6;

s₁ is 0, 1, 2, 3, 4, 5 or 6;

n₉ is 1, 2, 3, 4, 5, 6, 8, 12 or 24;

t is 0, 1, or 2;

R₄₀ is —SO₃H, —COOH, —C(O)NH(CH₂)₂SO₃H, or —C(O)NH(CH₂)₂COOH; and

each x independently is 2 or 3.

In some embodiments, when D is

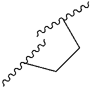 [SMILES: *CC*]

and s is 0, and T is —(CH₂)_{3 or 4}—, then E is not E3 wherein X₄ is N, Y₂ is O or S, Z₂ is CH, t is 0, 1, or 2, and R₅ is fluoro.

In some embodiments, when s is 1 and E is E3, then t is not 0, and R₈ is not C₁₋₄ alkyl, —C(O)—O—C₁₋₄ alkyl, 3- to 14-membered heterocycloalkyl, or —O—(CH₂)₁₋₄-(3- to 14-membered heterocycloalkyl).

In some embodiments, when s is 1 and E is E4 or E5 wherein X₄ is CH, Y₂ is O or S, and Z₂ is CH, then t is not 0, and R₈ is not C₁₋₄ alkyl, —C(O)—O—C₄ alkyl, 3- to 14-membered heterocycloalkyl, or —O—(CH₂)₁₋₄-(3- to 14-membered heterocycloalkyl).

In some embodiments, when s is 0, E is E1, and G is —NR₁₃R₁₄ wherein one of R₁ 3 and R₁₄ is H, then the other is not a 5- to 9-membered heteroaryl or phenyl.

The PBD drug moiety of Formula (IV) can have one or more of the following features when applicable:

In some embodiments, the PBD drug moiety of Formula (IV) is of Formula (IV-a),

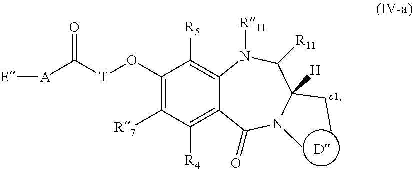

a tautomer thereof, a pharmaceutically acceptable salt or solvate thereof, or a pharmaceutically acceptable salt or solvate of the tautomer.

In some embodiments, the conjugates of Formulae (IV-a) include those where each of the moieties defined for one of E″, A, R₄, R₅, R″₇, R″₁₀, R₁₁, and D″ can be combined with any of the moieties defined for the others of E″, A, R₄, R₅, R″₇, R″₁₀, R₁₁, and D″.

In some embodiments, D′ is D1.

In some embodiments, D′ is D2.

In some embodiments, D′ is D3.

In some embodiments, D′ is D4.

In some embodiments, the PBD drug moiety of Formula (IV) is of any one of formulae (V-1), (V-2), and (V-3):

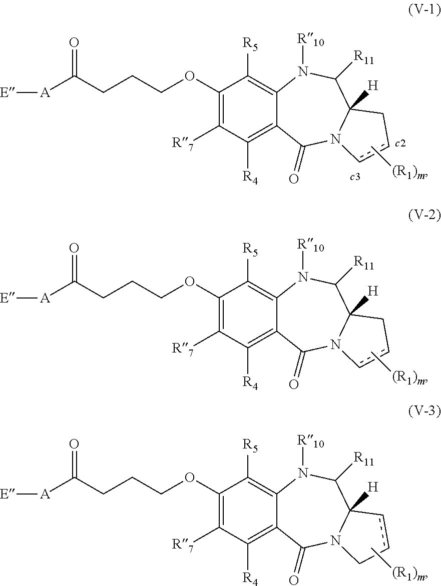

a tautomer thereof, a pharmaceutically acceptable salt or solvate thereof, or a pharmaceutically acceptable salt or solvate of the tautomer.

In some embodiments, the conjugates of any one of Formulae (V-1)-(V-3) include those where each of the moieties defined for one of E″, A, R₁, R₄, R₅, R″₇, R″₁₀, R₁₁, and m can be combined with any of the moieties defined for the others of E″, A, R₁, R₄, R₅, R″₇, R″₁₀, R₁₁, and m.

In some embodiments, the PBD drug moiety of Formula (IV) is of Formula (VI-1):

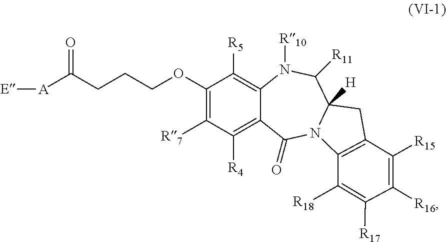

a tautomer thereof, a pharmaceutically acceptable salt or solvate thereof, or a pharmaceutically acceptable salt or solvate of the tautomer.

In some embodiments, the conjugates of Formula (VI-1) include those where each of the moieties defined for one of E″, A, R₄, R₅, R″₇, R″₁₀, R₁₁, R₁₅, R₁₆, R₁₇, and R₁₈ can be combined with any of the moieties defined for the others of E″, A, R₄, R₅, R″₇, R″₁₀, R₁₁, R₁₅, R₁₆, R₁₇, and R₁₈.

In some embodiments, the PBD drug moiety of Formula (IV) is of Formula (VII), (VII-1), (VII-2), or (VII-3):

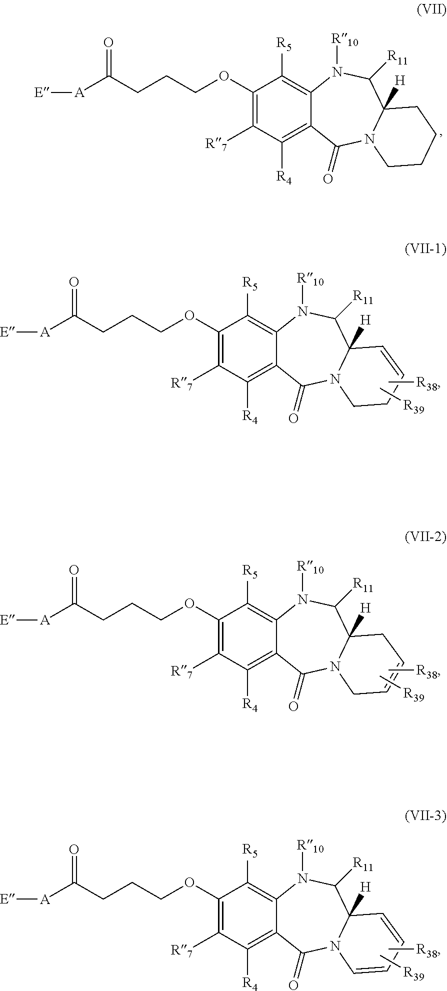

a tautomer thereof, a pharmaceutically acceptable salt or solvate thereof, or a pharmaceutically acceptable salt or solvate of the tautomer.

In some embodiments, the conjugates of any one of Formulae (VII), (VII-1), (VII-2), and (VII-3) include those where each of the moieties defined for one of E″, A, R₄, R₅, R″₇, R″₁₀, R₁₁, R₃₈, and R₃₉, where applicable, can be combined with any of the moieties defined for the others of E″, A, R₄, R₅, R″₇, R″₁₀, R₁₁, R₃₈, and R₃₉.

In some embodiments the PBD drug moiety of Formula IV is of Formula VII):

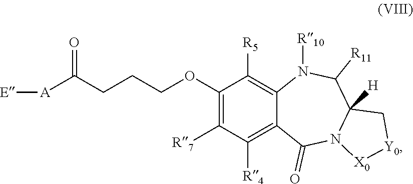

a tautomer thereof, a pharmaceutically acceptable salt or solvate thereof, or a pharmaceutically acceptable salt or solvate of the tautomer.

In some embodiments, the conjugates of Formula (VIII) include those where each of the moieties defined for one of E″, A, R₄, R₅, R″₇, R″₁₀, R₁₁, X₀, and Y₀ can be combined with any of the moieties defined for the others of E″, A, R₄, R₅, R″₇, R″₁₀, R₁₁, X₀, and Y₀.

In some embodiments, when D′ is D1, the dotted line between C₂ and C₃ is a double bond, and m is 1, then R₁ is C₆₋₁₀ aryl group, optionally substituted by one or more substituents selected from —OH, halo, —NO₂, —CN, —N₃, —OR₂, —COOH, —COOR₂, —COR₂, —OCONR₁₃R₁₄. C₁₋₁₀ alkyl, C₃₋₁₀ cycloalkyl, C₂₋₁₀ alkenyl, C₂₋₁₀ alkynyl, a polyethylene glycol unit —(OCH₂CH₂)ᵣ—ORₐ, 3- to 14-membered heterocycloalkyl, 5- to 12-membered heteroaryl, bis-oxy-C₁₋₃ alkylene, —NR₁₃R₁₄, —S(═O)₂R₁₂, —S(═O)₂NR₁₃R₁₄, —SR₂, —SOₓM, —OSOₓM, —NR₉COR₁₉, and —NH(C═NH)NH₂.

In some embodiments, when D′ is D1, the dotted line between C₂ and C₃ is a double bond, and m is 1, then R₁ is C₆₋₁₀ aryl group, optionally substituted by one or more substituents selected from —OH, halo, —NO₂, —CN, —N, —OR₂, —COOH, —COOR₂, —COR₂, —OCONR₁₃R₁₄, C₁₋₁₀ alkyl, C₃₋₁₀ cycloalkyl, C₂₋₁₀ alkenyl, C₂₋₁₀ alkynyl, a polyethylene glycol unit —(OCH₂CH₂)ᵣ—ORₐ, 3- to 14-membered heterocycloalkyl, 5- to 12-membered heteroaryl, —NR₁₃R₁₄, —S(═O)₂R₁₂, —S(═O)₂NR₁₃R₁₄, —SR₂, —NR₉COR₁₉, and —NH(C═NH)NH₂.

In some embodiments, when D′ is D1, the dotted line between C₂ and C₃ is a double bond, and m is 1, then R₁ is C₆₋₁₀ aryl group, optionally substituted by one or more substituents selected from —OH, halo, —OR₂, —COOH, —COOR₂, —COR₂, 3- to 14-membered heterocycloalkyl, and —NR₁₃R₁₄.

In some embodiments, when D′ is D1, the dotted line between C₂ and C₃ is a double bond, and m is 1, then R₁ is C₆₋₁₀ aryl group, substituted by one or more substituents selected from —OH, halo, —OR₂, —COOH, —COOR₂, —COR₂, 3- to 14-membered heterocycloalkyl, and —NR₁₃R₁₄.

In some embodiments, when D′ is D1, the dotted line between C₂ and C₃ is a double bond, and m is 1, then R₁ is C₆₋₁₀ aryl group, substituted by one substituent selected from —OH, halo, —OR₂, —COOH, —COOR₂, —COR₂, 3- to 14-membered heterocycloalkyl, and —NR₁₃R₁₄.

In some embodiments, when D′ is D1, the dotted line between C₂ and C₃ is a double bond, and m is 1, then R₁ is C₆₋₁₀ aryl group, substituted by one substituent selected from —OH, —OR₂, —COOH, —COOR₂, 3- to 14-membered heterocycloalkyl, and —NR₁₃R₁₄.

In some embodiments, when D′ is D1, the dotted line between C₂ and C₃ is a double bond, and m is 1, then R₁ is C₆₋₁₀ aryl group, substituted by one substituent selected from —OH, and —COOH.

In some embodiments, when D′ is D1, the dotted line between C₂ and C₃ is a double bond, and m is 1, then R₁ is C₆₋₁₀ aryl group, substituted by one substituent selected from —OR₂— and —COOR₂.

In some embodiments, when D′ is D1, the dotted line between C₂ and C₃ is a double bond, and m is 1, then R₁ is C₆₋₁₀ aryl group, substituted by one 3- to 14-membered heterocycloalkyl.

In some embodiments, when D′ is D1, the dotted line between C₂ and C₃ is a double bond, and m is 1, then R₁ is C₆₋₁₀ aryl group, substituted by one —NR₁₃R₁₄.

In some embodiments, when D′ is D1, the dotted line between C₂ and C₃ is a double bond, and m is 1, then R₁ is C₁₋₈ alkyl.

In some embodiments, when D′ is D1, the dotted line between C₂ and C₃ is a double bond, and m is 1, then R₁ is C₃₋₆ cycloalkyl.

In some embodiments, when D′ is D1, the dotted line between C₂ and C₃ is a double bond, and m is 1, then R₁ is cyclopropyl.

In some embodiments, when D′ is D1, the dotted line between C₂ and C₃ is a double bond, and m is 1, then R₁ is

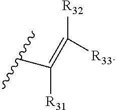

In some embodiments, when D′ is D1, the dotted line between C₂ and C₃ is a double bond, and m is 1, then R₁ is

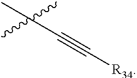

In some embodiments, when D′ is D1, the dotted line between C₂ and C₃ is a double

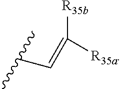

bond, and m is 1, then R₁ is

In some embodiments, when D′ is D1, the dotted line between C₂ and C₃ is a double bond, and m is 1, then R₁ is

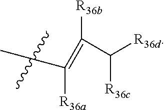

In some embodiments, when D′ is D1, the dotted line between C₂ and C₃ is a double bond, and m is 1, then R₁ is halo.

In some embodiments, when D′ is D1, the dotted line between C₂ and C₃ is a single bond, and m is 1, then R₁ is: —OH, ═O, ═CH₂, —CN, —R₂, —OR₂, halo, ═CH—R₆, ═C(R₆)₂, —O—SO₂R₂, —CO₂R₂, —COR₂, —CHO, or —COOH.

In some embodiments, when D′ is D1, the dotted line between C₂ and C₃ is a single bond, and m is 1, then R₁ is: ═CH₂, ═CH—R₆ or ═C(R₆)₂.

In some embodiments, when D′ is D1 and m is 2, then each R₁ independently is halo and either both R₁ are attached to the same carbon atom or one is attached to C₂ and the other is attached to C₃.

In some embodiments, when D′ is D4, the dotted line is a single bond, and R₃₈ and R₃₉ are each hydrogen.

In some embodiments, T is C₂₋₆ alkylene linker.

In some embodiments, T is C₂— alkylene linker.

In some embodiments, T is butylene.

In some embodiments, T is propylene.

In some embodiments, T is n-propylene.

In some embodiments, T is ethylene.

In some embodiments, each

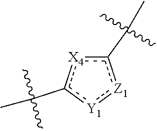

independently is

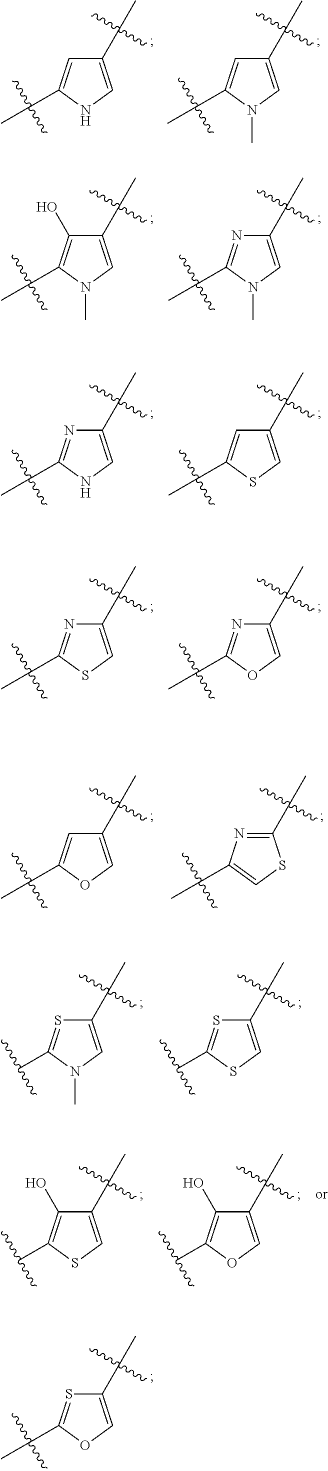

In some embodiments, each

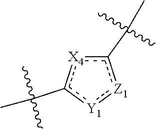

independently is

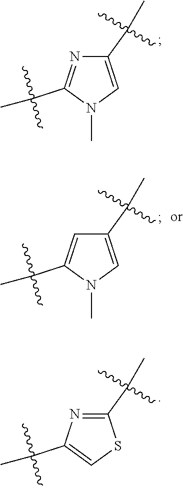

In some embodiments, s is 0, 1, 2 or 3.

In some embodiments, s is 0, 1 or 2.

In some embodiments, s is 1, 2 or 3.

In some embodiments, s is 0 or 1.

In some embodiments, s is 1 or 2.

In some embodiments, s is 2 or 3.

In some embodiments, s is 0.

In some embodiments, s is 0, and A is a single bond.

In some embodiments, s is 1.

In some embodiments, s is 2.

In some embodiments, A is

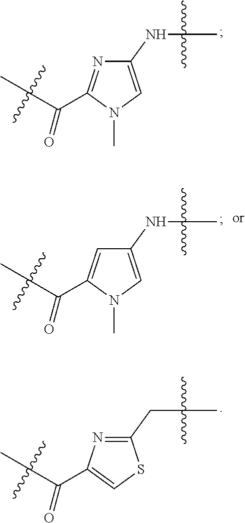 [SMILES: *Nc1cn(C)c(C(*)=O)n1 ; *Nc1cc(C(*)=O)n(C)c1 ; *Cc1nc(C(*)=O)cs1]

wherein each X₁ independently is CH or N.

In some embodiments, A is

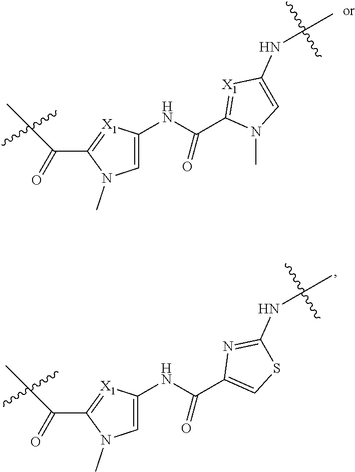

wherein each X₁ independently is CH or N.

In some embodiments, A is

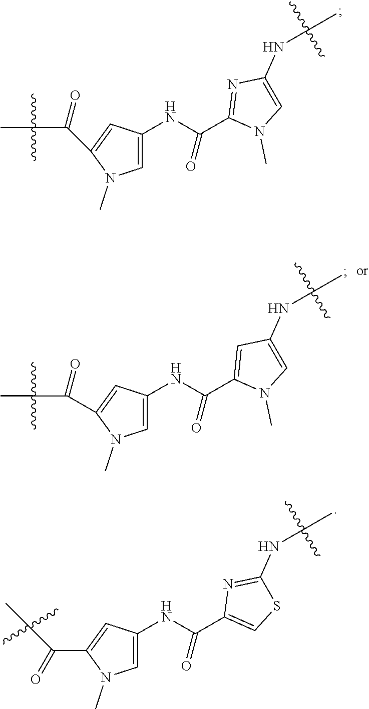 [SMILES: *Nc1cn(C)c(C(=O)Nc2cc(C(*)=O)n(C)c2)n1 ; *Nc1cc(C(=O)Nc2cc(C(*)=O)n(C)c2)n(C)c1 ; *Nc1nc(C(=O)Nc2cc(C(*)=O)n(C)c2)cs1]

In some embodiments, A is

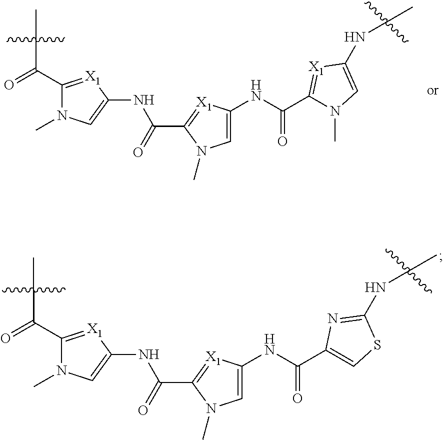

wherein each X₁ independently is CH or N.

In some embodiments, A is:

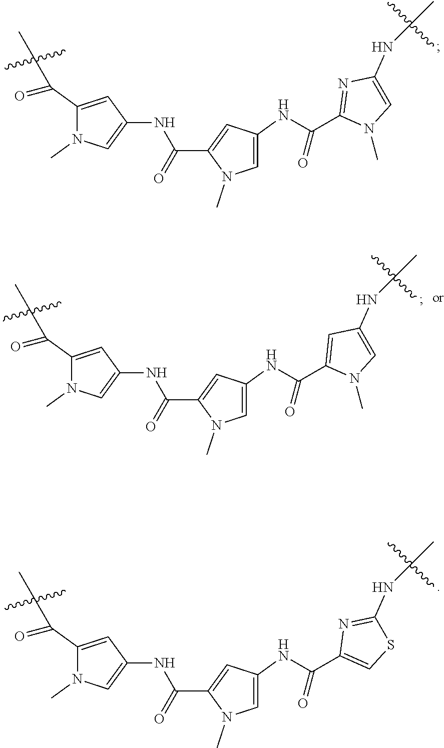 [SMILES: *Nc1cn(C)c(C(=O)Nc2cc(C(=O)Nc3cc(C(*)=O)n(C)c3)n(C)c2)n1 ; *Nc1cc(C(=O)Nc2cc(C(=O)Nc3cc(C(*)=O)n(C)c3)n(C)c2)n(C)c1 ; *Nc1nc(C(=O)Nc2cc(C(=O)Nc3cc(C(*)=O)n(C)c3)n(C)c2)cs1]

In some embodiments, A is:

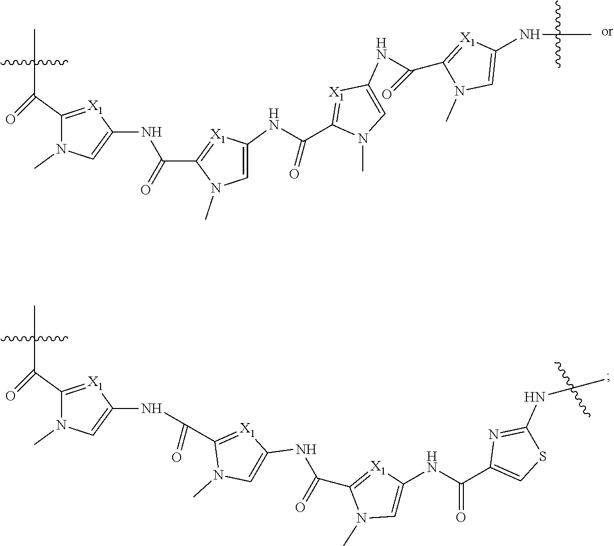

wherein each X₁ independently is CH or N.

In some embodiments, A is:

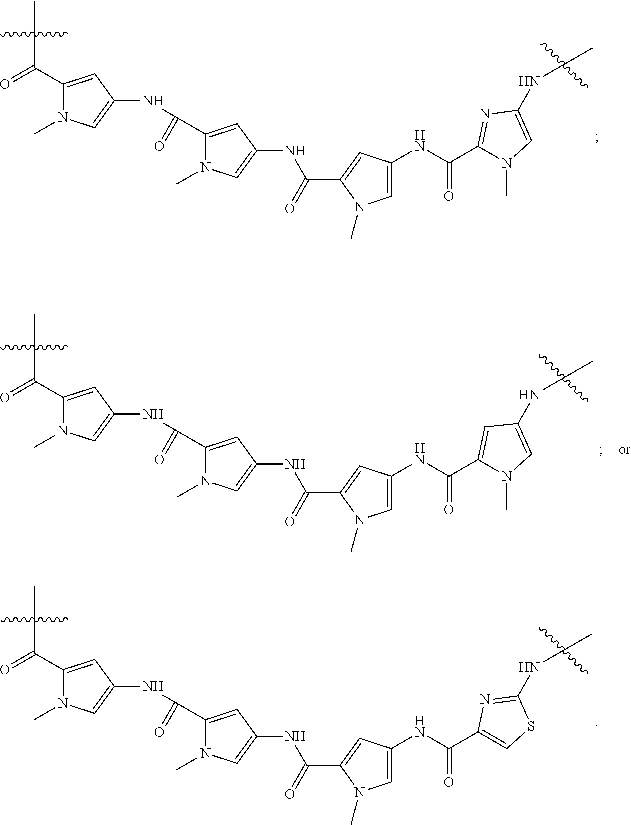 [SMILES: *Nc1cn(C)c(C(=O)Nc2cc(C(=O)Nc3cc(C(=O)Nc4cc(C(*)=O)n(C)c4)n(C)c3)n(C)c2)n1 ; *Nc1cc(C(=O)Nc2cc(C(=O)Nc3cc(C(=O)Nc4cc(C(*)=O)n(C)c4)n(C)c3)n(C)c2)n(C)c1 ; *Nc1nc(C(=O)Nc2cc(C(=O)Nc3cc(C(=O)Nc4cc(C(*)=O)n(C)c4)n(C)c3)n(C)c2)cs1]

In some embodiments, E is

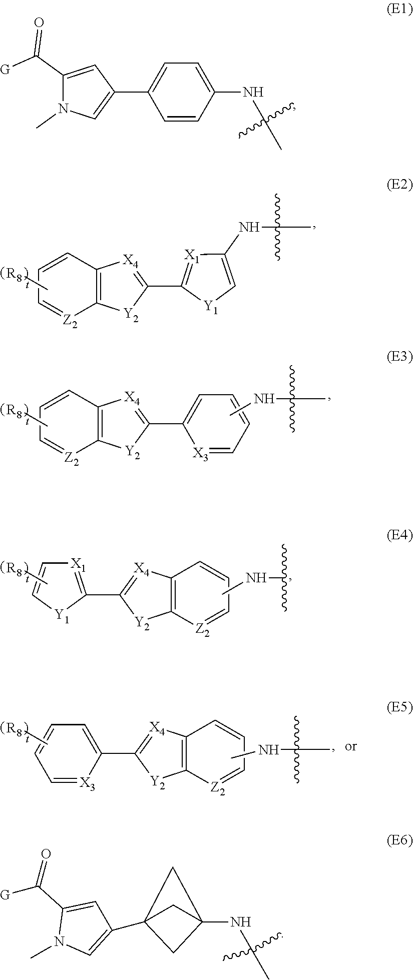

In some embodiments, t is 0.

In some embodiments, t is 1.

In some embodiments, t is 2.

In some embodiments, E is

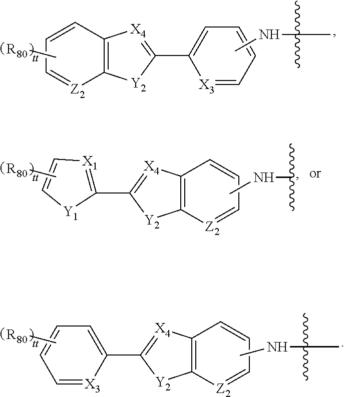

In some embodiments, tt is 1.

In some embodiments, tt is 2.

In some embodiments G is —OH.

In some embodiments, G is —NH—(C₁₋₆ alkylene)-OH, wherein C₁₋₆ alkylene is a linear or branched alkylene.

In some embodiments, G is —NH—(CH₂), —OH, in which u is 1, 2, 3, 4, 5, or 6.

In some embodiments, G is —NH—(CH₂)—OH, in which u is 2, 3, 4, 5, or 6.

In some embodiments, G is —NH—(CH₂)₃—OH.

In some embodiments, G is —NH—CH₂—CH(CH₃)—OH.

In some embodiments, G is —NR₁₃R₁₄, wherein each of R₁₃ and R₁₄ are each independently H, C₁₋₁₀ alkyl, 3- to 20-membered heterocycloalkyl, 5- to 20-membered heteroaryl, or C₆₋₂₀ aryl.

In some embodiments, G is —NR₁₃R₁₄, and one of R₁₃ and R₁₄ is H, then the other is H, C₁₋₁₀ alkyl, C₃₋₁₀ cycloalkyl, or 3- to 20-membered heterocycloalkyl.

In some embodiments, G is —NR₁₃R₁₄, wherein each of R₁₃ and R₁₄ independently is H or C₁₋₁₀ alkyl.

In some embodiments, G is —O—(CH₂)₃—NH₂.

In some embodiments, G is —O—CH(CH₃)—(CH₂)₂—NH₂.

In some embodiments, G is —NH—(CH₂)₃—O—C(═O)—CH(CH₃)—NH₂.

In some embodiments, G is —NHR₁₄

In some embodiments, G is —NH₂.

In some embodiments G is

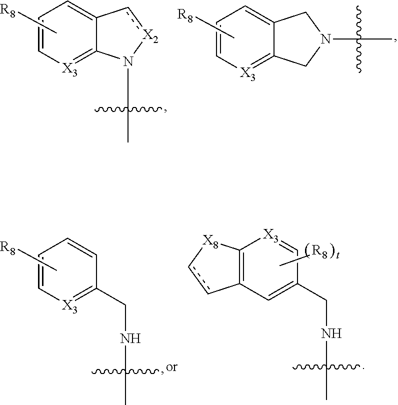

In some embodiments, G is

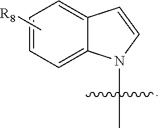

In some embodiments, G is

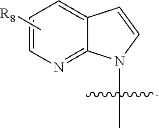

In some embodiments, G is

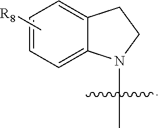

In some embodiments, G is

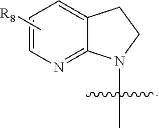

In some embodiments, G is

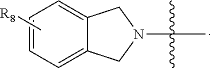

In some embodiments, G is

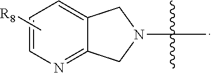

In some embodiments, G is

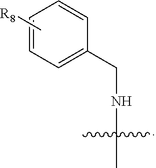

In some embodiments, G is

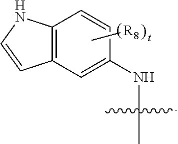

In some embodiments, G is

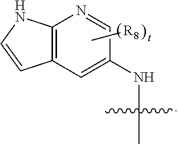

In some embodiments, E is

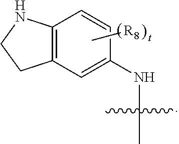

In some embodiments, E is

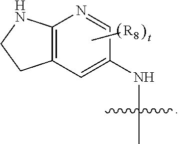

In some embodiments, E is

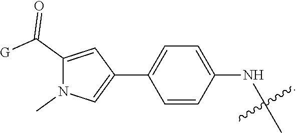

In some embodiments, E is

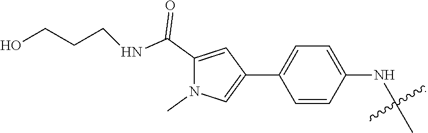 [SMILES: *Nc1ccc(-c2cc(C(=O)NCCCO)n(C)c2)cc1]

In some embodiments, E is

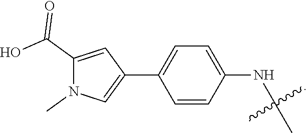 [SMILES: *Nc1ccc(-c2cc(C(=O)O)n(C)c2)cc1]

In some embodiments, E is

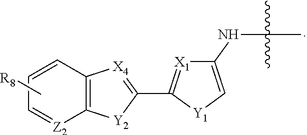

In some embodiments, E is

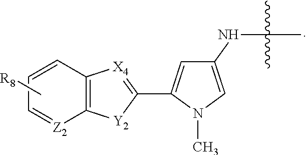

In some embodiments, E is

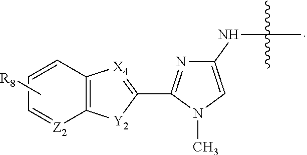

In some embodiments, E is

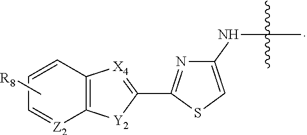

In some embodiments, E

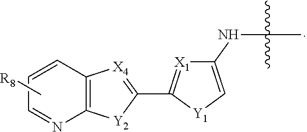

In some embodiments, E is

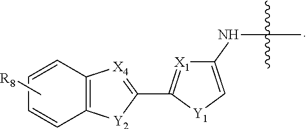

In some embodiments, E is

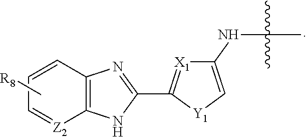

In some embodiments, E is

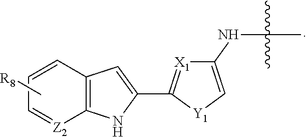

In some embodiments, E is

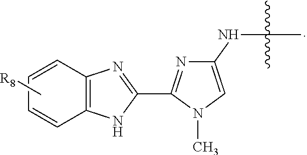

In some embodiments, E is

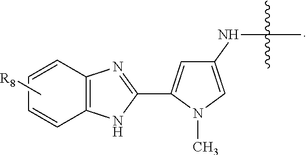

In some embodiments, E is

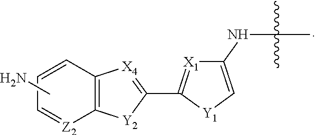

In some embodiments, E is

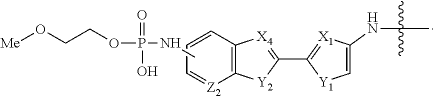

In some embodiments, E is

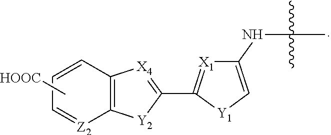

In some embodiments, E is

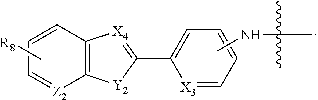

In some embodiments, E is

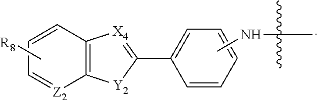

In some embodiments, E is

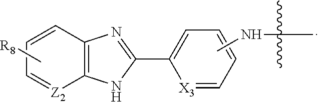

In some embodiments, E is

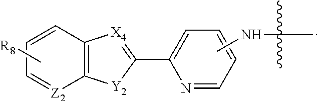

In some embodiments, E is

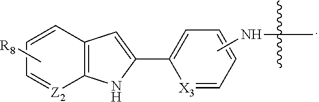

In some embodiments, E is

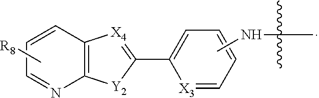

In some embodiments, E is

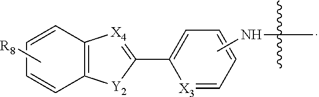

In some embodiments, E is

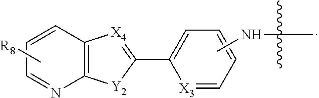

In some embodiments, E is

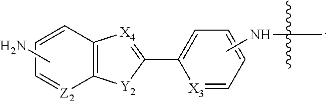

In some embodiments, E is

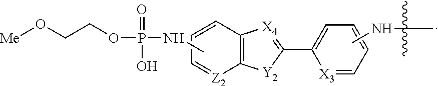

In some embodiments, E is

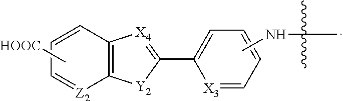

In some embodiments, E is

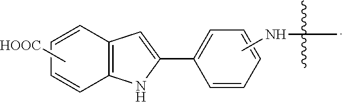

In some embodiments, E is

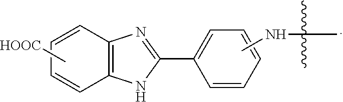

In some embodiments, E is

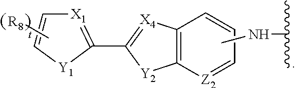

In some embodiments, E is

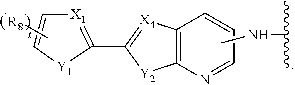

In some embodiments, E is

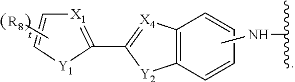

In some embodiments, E is

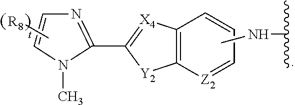

In some embodiments, E is

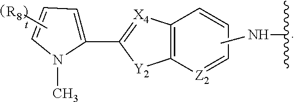

In some embodiments, E is

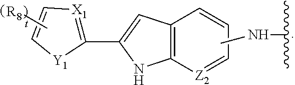

In some embodiments, E is

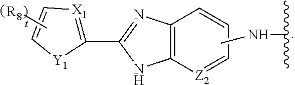

In some embodiments, E is

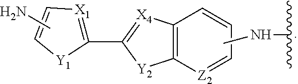

In some embodiments, E is

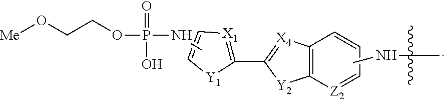

In some embodiments, E is

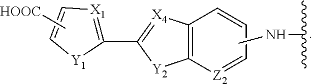

In some embodiments, E is

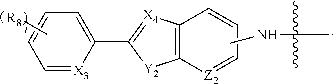

In some embodiments, E is

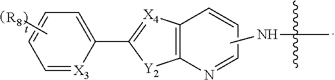

In some embodiments, E is

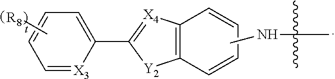

In some embodiments, E is

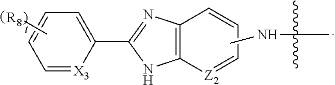

In some embodiments, E is

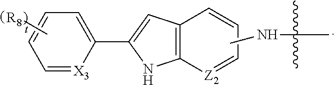

In some embodiments, E is

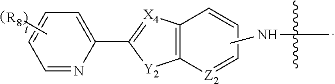

In some embodiments, E is

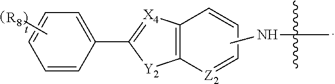

In some embodiments, E is

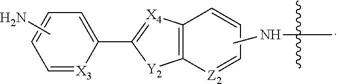

In some embodiments, E is

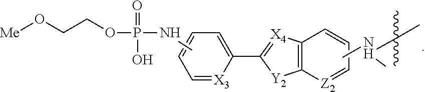

In some embodiments, E is

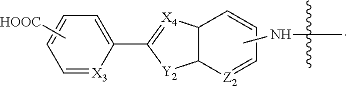

In some embodiments, E is

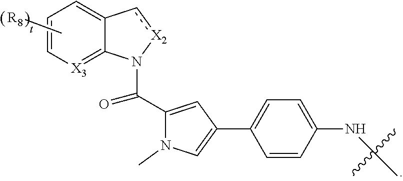

In some embodiments, E is

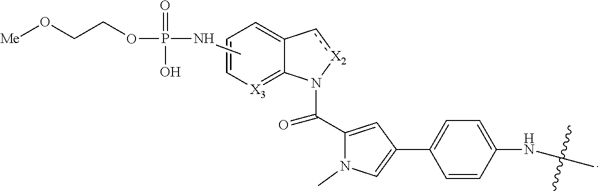

In some embodiments, E is

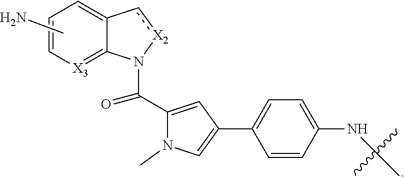

In some embodiments, E is

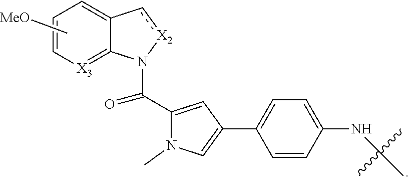

In some embodiments, E is

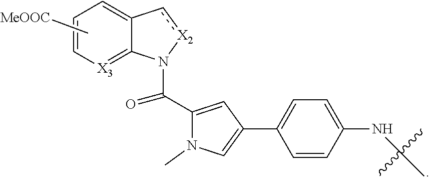

In some embodiments, E is

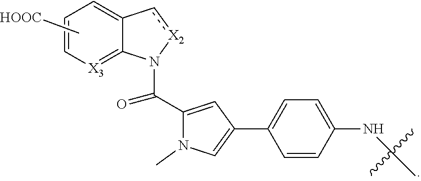

In some embodiments, E is

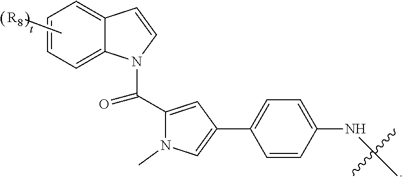

In some embodiments, E is

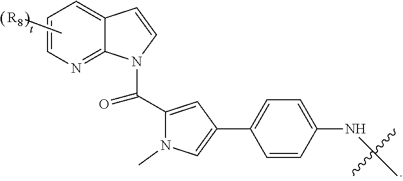

In some embodiments, E is

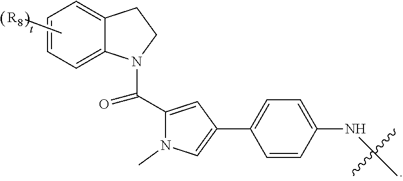

In some embodiments, E is

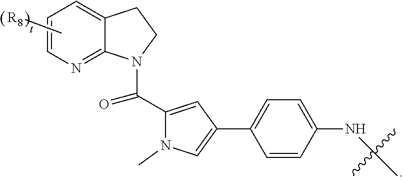

In some embodiments, E is

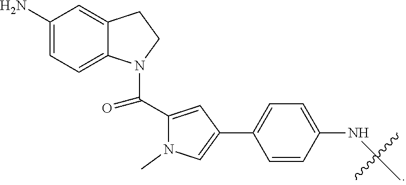 [SMILES: *Nc1ccc(-c2cc(C(=O)N3CCc4cc(N)ccc43)n(C)c2)cc1]

In some embodiments, E is

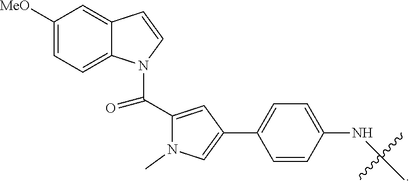 [SMILES: *Nc1ccc(-c2cc(C(=O)n3ccc4cc(OC)ccc43)n(C)c2)cc1]

In some embodiments, E is

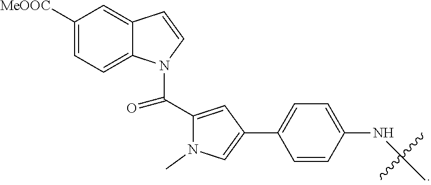 [SMILES: *Nc1ccc(-c2cc(C(=O)n3ccc4cc(C(=O)OC)ccc43)n(C)c2)cc1]

In some embodiments, E is

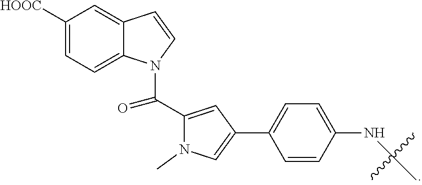 [SMILES: *Nc1ccc(-c2cc(C(=O)n3ccc4cc(C(=O)O)ccc43)n(C)c2)cc1]

In some embodiments, E is

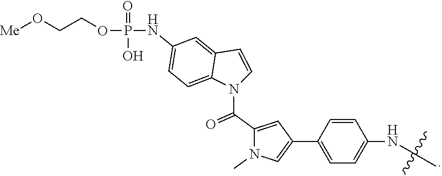 [SMILES: *Nc1ccc(-c2cc(C(=O)n3ccc4cc(NP(=O)(O)OCCOC)ccc43)n(C)c2)cc1]

In some embodiments, E is

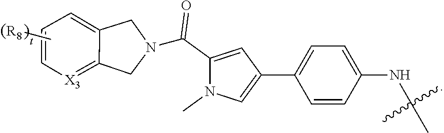

In some embodiments, E is

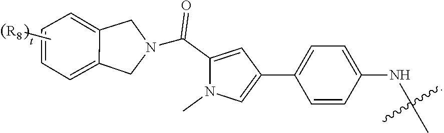

In some embodiments, E is

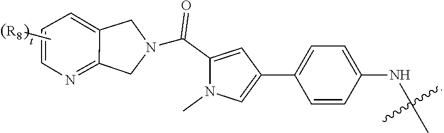

In some embodiments, E is

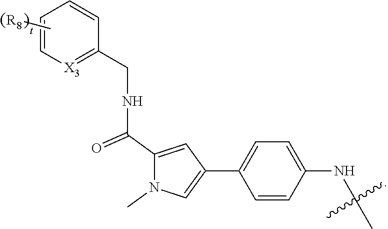

In some embodiments, E is

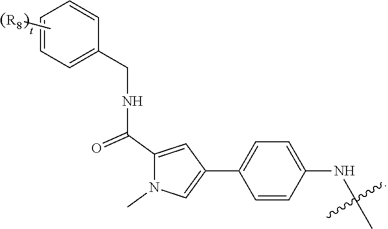

In some embodiments, E is

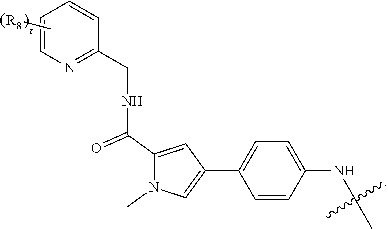

In some embodiments, E is

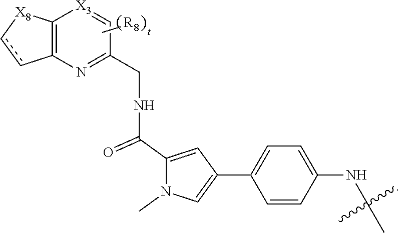

In some embodiments, E is

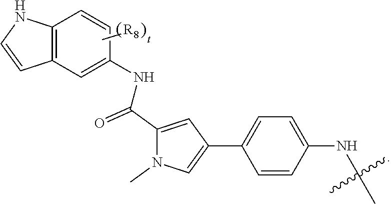

In some embodiments, E is

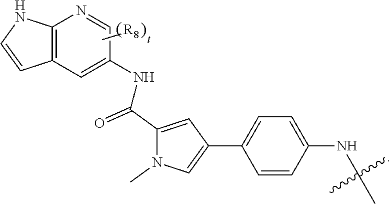

In some embodiments, E is

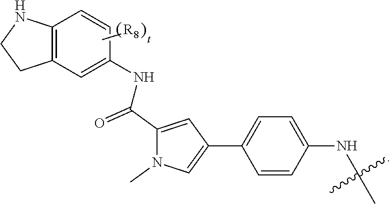

In some embodiments, E is

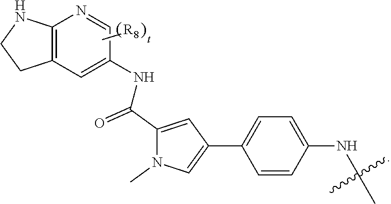

In some embodiments, E is

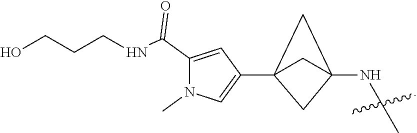 [SMILES: *NC12CC(c3cc(C(=O)NCCCO)n(C)c3)(C1)C2]

In some embodiments, E is

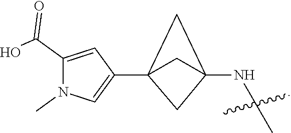 [SMILES: *NC12CC(c3cc(C(=O)O)n(C)c3)(C1)C2]

In some embodiments, E is

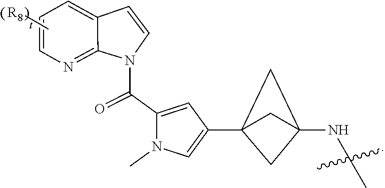

In some embodiments, E is

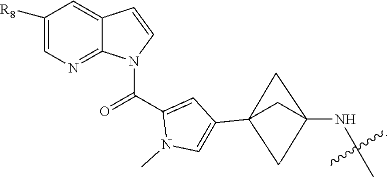

In some embodiments, E is

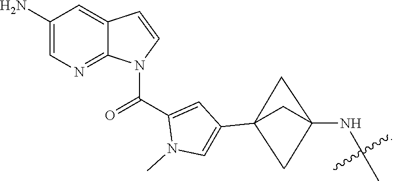 [SMILES: *NC12CCCC(c3cc(C(=O)n4ccc5cc(N)cnc54)n(C)c3)(C1)C2]

In some embodiments, E is

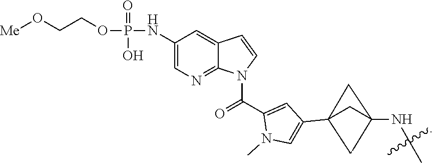 [SMILES: *NC12CCCC(c3cc(C(=O)n4ccc5cc(NP(=O)(O)OCCOC)cnc54)n(C)c3)(C1)C2]

In some embodiments, E is

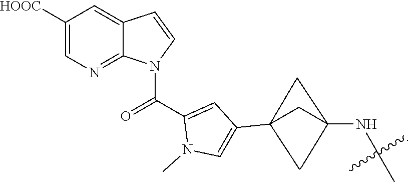 [SMILES: *NC12CCCC(c3cc(C(=O)n4ccc5cc(C(=O)O)cnc54)n(C)c3)(C1)C2]

In some embodiments, E is

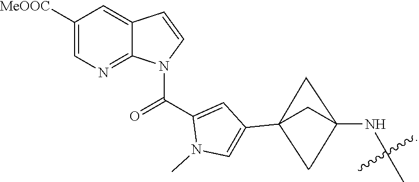 [SMILES: *NC12CCCC(c3cc(C(=O)n4ccc5cc(C(=O)OC)cnc54)n(C)c3)(C1)C2]

In some embodiments, E is

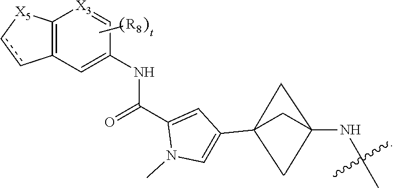

In some embodiments, E is

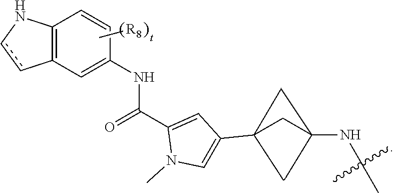

In some embodiments, E is

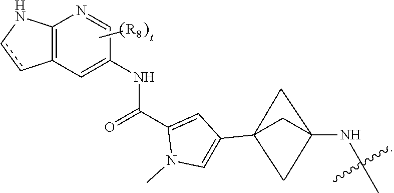

In some embodiments, in

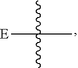

the functional group of E is G or a portion thereof.

In some embodiments, in

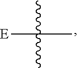

the

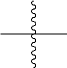

denotes direct or indirect linkage to the PBRM via G or a portion thereof.

In some embodiments, in

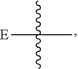

the

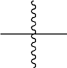

denotes direct or indirect linkage to L^{C} via G or a portion thereof.

In some embodiments, in

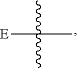

the

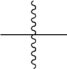

denotes direct or indirect linkage to L^{D} via G or a portion thereof.

In some embodiments, in

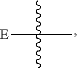

the functional group of E is R₈ or a portion thereof.

In some embodiments, in

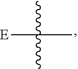

the

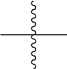

denotes direct or indirect linkage to the PBRM via R₈ or a portion thereof.

In some embodiments, in

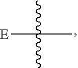

the

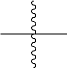

denotes direct or indirect linkage to L^{C} via R₈ or a portion thereof.

In some embodiments, in

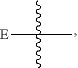

the

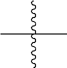

denotes direct or indirect linkage to L^{D} via R₈ or a portion thereof.

In some embodiments, each R₈ independently is —OH, halo, —NO₂, —CN, —N₃, —OR₂, —COOH, —COOR₂, —COR₂, —OCONR₁₃R₁₄, —CO—NH—(C₁₋₆ alkylene)-R₁₃ₐ, —OCO—NH—(C₁₋₆ alkylene)-R₁₃ₐ, C₁₋₁₀ alkyl, C₃₋₁₀ cycloalkyl, C₂₋₁₀ alkenyl, C₂₋₁₀ alkynyl, a polyethylene glycol unit —(OCH₂CH₂)ᵣ—ORₐ, 3- to 14-membered heterocycloalkyl, 5- to 12-membered heteroaryl, —S(═O)₂R₁₂, —S(═O)₂NR₁₃R₁₄, —SR₂, —SOₓM, —OSOₓM, —NR₉COR₁₉, —NH(C═NH)NH₂, —R₂₀-R₂₁—NR₁₃R₁₄, —R₂₀-R₂₁—NH—P(O)(OH)—(OCH₂CH₂)ₙ₉—OCH₃, or —O—P(O)(OH)—(OCH₂CH₂)ₙ₉—OCH₃.

In some embodiments, each R₈ independently is —CONR₁₃R₁₄.

In some embodiments, when E is

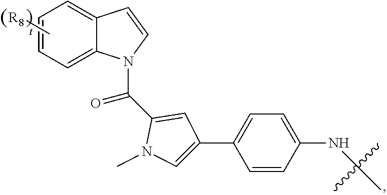

then at least one R₈ is —CONR₁₃R₁₄.

In some embodiments, when E is

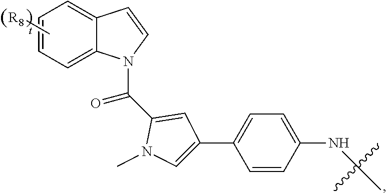

then at least one R₈ is —R₂₀-R₂₁—NH—P(OOH)—(OCH₂CH₂)ₙ₉—OCH₃.

In some embodiments, when E is

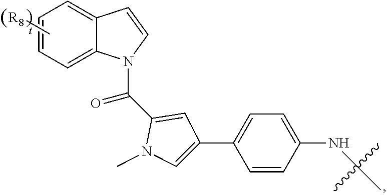

then at least one R₈ is —O—P(O)(OH)—(OCH₂CH₂)ₙ₉—OCH₃.

In some embodiments, each R₅ independently is —CO—NH—(C₁₋₆ alkylene)-R₁₃ₐ or —OCO—NH—(C₁₋₆ alkylene)-R₁₃ₐ.

In some embodiments, when E is

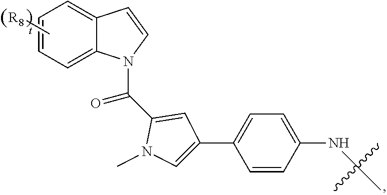

then at least one R₈ is —CO—NH—(C₁₋₆ alkylene)-R₁₃ₐ or —OCO—NH—(C₆ alkylene)-R₃ₐ.

In some embodiments, each R₅ independently is —OH, halo, —NO₂, —CN, —N₃, —OR₂, —COOH, —COOR₂, —COR₂, —OCONR₁₃R₁₄, C₁₋₁₀ alkyl, C₃₋₁₀ cycloalkyl, C₂₋₄ alkenyl, C₂₋₁₀ alkynyl, a polyethylene glycol unit —(OCH₂CH₂)ᵣ—ORₐ, 3- to 14-membered heterocycloalkyl, 5- to 12-membered heteroaryl, —S(═O)₂R₁₂, —S(═O)₂NR₁₃R₁₄, —SR₁₂, —SOₓM, —OSOₓM, —NR₉COR₁₀, —NH(C═NH)NH₂, —R₂₀-R₂₁—NR₁₃R₁₄, —R₂₀-R₂₁—NH—P(O)(OH)—(OCH₂CH₂)ₙ₉—OCH₃, or —O—P(O)(OH)—(OCH₂CH₂)ₙ₉—OCH₃.

In some embodiments, each R₅ independently is —OH, —OR₂, —COOH, —COOR₂, —COR₂, —OCONR₁₃R₁₄, —CONR₁₃R₁₄, —CO—NH—(C₁₋₆ alkylene)-R₁₃ₐ, a polyethylene glycol unit —(OCH₂CH₂)ᵣ—ORₐ, 3- to 7-membered heterocycloalkyl, 5- to 6-membered heteroaryl, —S(═O)₂R₂, —S(═O)₂NR₁₃R₁₄, —SR₁₂—R₂₀-R₂₁—NR₁₃R₁₄, —R₂₀-R₂₁—NH—P(O)(OH)—(OCH₂CH₂)ₙ—OCH₃, or —O—P(O)(OH)—(OCH₂CH₂)ₙ₉—OCH₃;

wherein R₁₃ and R₁₄ are each independently —H or C₁₋₁₀ alkyl;

each R₂₀ is phenylene; and

each R₂₁ independently is C₁₋₄ alkylene.

In some embodiments, each R₅ independently is —OH, —OR₂, —COO, —COOR₂, —COR₂, —S(═O)₂R₁₂, —SR₁₂, —R₂₀-R₂₁—NR₁₃R₁₄, —R₂₀-R₂₁—NH—P(O)(OH)—(OCH₂CH₂)ₙ₉—OCH₃, or —O—P(O)(OH)—(OCH₂CH₂)ₙ—OCH₃.

In some embodiments, each R₈ independently is —OH or —OR₂.

In some embodiments, each R₅ independently is —COH, —COOR₂, or —COR₂.

In some embodiments, each R₅ independently is —S(═O)₂R₂ or —SR₂.

In some embodiments, each R₈ independently is —CONR₁₃R₁₄ or —CO—NH—(C₁₋₆ alkylene)-R₁₃ₐ.

In some embodiments, each R₈ independently is —R₂₀-R₂₁—NR₁₃R₁₄.

In some embodiments, R₈ is —NH₂.

In some embodiments, R₈ is —CH₂NH₂.

In some embodiments, R₈ is —CH₂CH₂NH₂.

In some embodiments, R₈ is —CH₂CH₂CH₂NH₂.

In some embodiments, R₈ is —NH—P(O)(OH)—(OCH₂CH₂)ₙ₉—OCH₃.

In some embodiments, R₈ is —NH—P(O)(OH)—(OCH₂CH₂)—OCH₃.

In some embodiments, R₈₀ is —O—P(O)(OH)—(OCH₂CH₂)ₙ₉—OCH₃.

In some embodiments, R₈ is —O—P(O)(OH)—(OCH₂CH₂)—OCH₃.

In some embodiments, R₈₀ is —OH, halo, —NO₂, —CN, —N₃, —COOH, —COR₂, —OCONR₁₃R₁₄. C₃₋₁₀ cycloalkyl, C₂₋₁₀ alkenyl, C₂₋₁₀ alkynyl, a polyethylene glycol unit —(OCH₂CH₂)ᵣ—ORₐ, 5- to 12-membered heteroaryl, —S(═O)₂R₁₂, —S(═O)₂NR₁₃R₁₄, —SR₂, —SOₓM, —OSOₓM, —NR₉COR₁₉, —NH(C═NH)NH₂, —R₂₀-R₂₁—NR₁₃R₁₄, —R₂₀-R₂₁—NH—P(O)(OH)—(OCH₂CH₂)ₙ₉—OCH₃, or —O—P(O)(OH)—(OCH₂CH₂)ₙ₉—OCH₃.

In some embodiments, each R₈₀ is —OH, —COOH, —COR₂, —OCONR₁₃R₁₄, a polyethylene glycol unit —(OCH₂CH₂)—ORₐ, 5- to 12-membered heteroaryl, —S(═O)₂R₂, —S(═O)₂NR₁₃R₁₄, —SR₁₂, —R₂₀-R₂₁—NR₁₃R₁₄, —R₂₀-R₂₁—NH—P(O)(OH)—(OCH₂CH₂)ₙ₉—OCH₃, or —O—P(O)(OH)—(OCH₂CH₂)ₙ₉—OCH₃

wherein R₁₃ and R₁₄ are each independently —H or C₁₋₁₀ alkyl;

each R₂ is a bond or phenylene; and

each R₂₁ independently is a bond or C₁₋₄ alkylene.

In some embodiments, each R₈₀ independently is —OH, —COOH, —COR₂, —S(═O)₂R₁₂, —SR₁₂, —R₂₀-R₂₁—NR₁₃R₁₄, —R₂₀-R₂₁—NH—P(O)(OH)—(OCH₂CH₂)ₙ₉—OCH₃, or —O—P(O)(OH)—(OCH₂CH₂)ₙ₉—OCH₃.

In some embodiments, each R₈₀ independently is —OH.

In some embodiments, each R₈₀ independently is —COOH or —COR₂.

In some embodiments, each R₈₀ independently is —S(═O)₂R₁₂ or —SR₂.

In some embodiments, each R₈₀ independently is —R₂₀-R₂₁—NR₁₃R₁₄. In some embodiments, R₈₀ is —NH₂. In some embodiments, R₁₀ is —CH₂NH₂. In some embodiments, R₈₀ is —CH₂CH₂NH₂. In some embodiments, R₈₀ is —CH₂CH₂CH₂NH₂.

In some embodiments, R₈₀ is —NH—P(O)(OH)—(OCH₂CH₂)ₙ₉—OCH₃.

In some embodiments, R₈₀ is —NH—P(O)(OH)—(OCH₂CH₂)—OCH₃.

In some embodiments, R₈₀ is —O—P(O)(OH)—(OCH₂CH₂)ₙ₉—OCH₃.

In some embodiments, R₈₀ is —O—P(O)(OH)—(OCH₂CH₂)—OCH₃.

In some embodiments, each R₁₃ₐ independently is OH or NHR₃.

In some embodiments, each occurrence of R₁₃ is independently H or C₁₋₁₀ alkyl (e.g., C₁₋₆ alkyl).

In some embodiments, each occurrence of R₁₄ is independently H or C₁₋₁₀ alkyl (e.g., C₁₋₆ alkyl).

In some embodiments, each occurrence of R₃ is independently 3- to 20-membered (e.g., 4- to 14-membered) heterocycloalkyl or 5- to 20-membered (e.g., 5- to 10-membered) heteroaryl.

In some embodiments, each occurrence of R₁₄ is independently 3- to 20-membered (e.g., 4- to 14-membered) heterocycloalkyl or 5- to 20-membered (e.g., 5- to 10-membered) heteroaryl.

In some embodiments, R₄, R₅ and R₂ are each independently —H, —R₂, —OH, —OR₂, —SH, —SR₂, —NH₂, —NHR₂, —NR₂R₃, —NO₂, halo or a polyethylene glycol unit —(OCH₂CH₂)ᵣ—ORₐ.

In some embodiments, at least one of R₄, R₅ and R₂ is —OR₂.

In some embodiments, at least one of R₄, R₅ and R₂ is a polyethylene glycol unit —(OCH₂CH₂)ᵣ—ORₐ.

In some embodiments, at least two of R₄, R₅ and R₂ are —H.

In some embodiments, two of R₄, R₅ and R₇ are —H, and the other is —OR₂.

In some embodiments, two of R₄, R₅ and R₇ are —H, and the other is —OCH₃.

In some embodiments, R₄ and R₅ are each —H, and R₇ is —OCH₃.

In some embodiments, R₄ and R₅ are each —H, and R₇ is —(OCH₂CH)ᵣ—ORₐ.

In some embodiments, R₄ and R₇ together form bis-oxy-C₁₋₃ alkylene.

In some embodiments, each of R₂₀ and R₂₁ is a bond.

In some embodiments, one of R₂₀ and R₂₁ is a bond and the other is not a bond.

In some embodiments, R₂₀ is a bond and R₂₁ is not a bond.

In some embodiments, R₂₀ is a bond and R₂₁ is C₁₋₁₀ alkylene.

In some embodiments, R₂ is a bond and R₂₀ is not a bond.

In some embodiments, R₂ is a bond and R₂₀ is C₆₋₁₀ arylene, 3-14 membered heterocycloalkylene or 5- to 12-membered heteroarylene.

In some embodiments, R¹⁰ and R¹¹ taken together with the nitrogen atom and carbon atom to which they are respectively attached, form a N═C double bond.

In some embodiments, R¹⁰ is —H or a nitrogen protecting group, and R¹¹ is -QR^{Q}.

In some embodiments, R¹⁰ is —H and R¹¹ is -QR^{Q}.

In some embodiments, R¹⁰ is a nitrogen protecting group and R¹¹ is -QR^{Q}, wherein the nitrogen protecting group is allyloxycarbonyl (alloc), carbobenzyloxy (Cbz), p-methoxybenzyl carbonyl (Moz or MeOZ), acetyl (Ac), benzoyl (Bz), benzyl (Bn), trichloroethoxycarbonyl (Troc), t-butoxycarbonyl (BOC) or 9-fluorenylmethylenoxycarbonyl (Fmoc).

In some embodiments, R¹¹ is —OSOₓM.

In some embodiments, R¹¹ is —SOₓM.

In some embodiments, R¹¹ is —OH.

In some embodiments, R¹¹ is —OPO₃M.

In some embodiments, R¹¹ is —O(CH₂CH₂O)ₙ₉CH3.

In some embodiments, R¹¹ is —OC(O)O—(CH₂—CH₂—O)ₙ₉CH₃.

In some embodiments, R¹¹ is —OC(O)NH—(CH₂—CH₂—O)ₙ₉CH₃.

In some embodiments, R¹¹ is —(CH₂)ₙ—NH—C(O)—CH₂—O—CH₂—C(O)—NH—(CH₂—CH₂—O)ₙ₉CH₃.

In some embodiments, R¹¹ is —O-sugar moiety.

In some embodiments, R₁₅, R₁₆, R₁₇ and R₁₈ are each independently —H, —OH, halo, —NO₂, —CN, —N₃, —OR₂, —COOH, —COOR₂, —COR₂, —OCONR₁₃R₁₄, C₁₋₁₀ alkyl, C₃₋₁₀ cycloalkyl, C₂₋₁₀ alkenyl, C₂₋₁₀ alkynyl, a polyethylene glycol unit —(OCH₂CH)ᵣ—ORₐ, 3-14 membered heterocycloalkyl, 5- to 12-membered heteroaryl, —NR₁₃R₁₄, —S(═O)₂R₁₂, —S(═O)₂NR₁₃R₁₄, —SR₁₂ or —NH(C═NH)NH₂.

In some embodiments, at least one of R₁₅, R₁₆, R₁₇ and R₁₈ is —H.

In some embodiments, at least two of R₁₅, R₁₆, R₁₇ and R₁₈ is —H.

In some embodiments, at least three of R₁₅, R₁₆, R₁₇ and R₁₈ is —H.

In some embodiments, R₁₅, R₁₆, R₁₇ and R₁₈ are each —H or —NR₁₃R₁₄.

In some embodiments, at least one of R₁₅, R₁₆, R₁₇ and R₁₈ is —NR₁₃R₁₄.

In some embodiments, at least one of R₁₅, R₁, R₁₇ and R₁₈ is —NH₂.

In some embodiments, one of R₁₅, R₁₆, R₁₁ and R₁ is —NR₁₃R₁₄.

In some embodiments, one of R₁₅, R₁₆, R₁₇ and R₁₈ is —NH₂.

In some embodiments, R₁₆, R₁₇ and R₁₈ are each —H; and R₁₈ is —NH.

In some embodiments, R₁₅, R₁₇ and R₁₈ are each —H; and R₁₅ is —NH₂.

In some embodiments, R₁₅, R₁₆ and R₁₈ are each —H; and R₁₇ is —NH₂.

In some embodiments, R₁₅, R₁₆ and R₁ are each —H; and R₁₈ is —NH₂.

In some embodiments, X₀ is CH₂, NR₆, or C═O.

In some embodiments, Y₀ is O, CH₂, or NR₆.

In some embodiments, Z₀ is absent.

In some embodiments, Z₀ is (CH₂); and n is 1 or 2.

In some embodiments, when Q is S or NH, then R^{Q} is —H.

In some embodiments, when Q is S or NH, then R^{Q} is optionally substituted C₁₋₂ alkyl.

In some embodiments, when Q is O, then R^{Q} is —H.

In some embodiments, when Q is O, then R^{Q} is optionally substituted C₁₋₂ alkyl.

In some embodiments, when Q is O, then R^{Q} is —SOₓM.

In some embodiments, when Q is O, then R^{Q} is hydrogen.

In some embodiments, when Q is O, then R^{Q} is —PO₃M.

In some embodiments, when Q is O, then R^{Q} is —(CH₂—CH₂—O)ₙ₉CH₃, and n₉ is 6, 8, 12 or 24.

In some embodiments, when Q is O, then R is —(CH₂—CH₂O)ₙ₉—(CH₂)₂—R₄₀. and n₉ is 6, 8, 12 or 24.

In some embodiments, when Q is O, then R^{Q} is —C(O)—(CH₂—CH₂—O)ₙ₉CH₃ and n₉ is 6, 8, 12 or 24.

In some embodiments, when Q is O, then R^{Q} is —C(O)—(CH₂—CH₂-0)ₙ₉CH₃ and n₉ is 6, 8, 12 or 24.

In some embodiments, when Q is O, then R^{Q} is —C(O)NH—(CH₂—CH₂—O)ₙ₉CH₃ and n₉ is 6, 8, 12 or 24.

In some embodiments, when Q is O, then R^{Q} is —(CH₂)—NH—C(O)—CH₂—O—CH₂—C(O)—NH—(CH₂—CH₂—O)ₙ₉CH₃, and n is 2 and n₉ is 6, 8, 12 or 24.

In some embodiments, when Q is O, then R^{Q} is —(CH₂)ₙ—NH—C(O)—(CH₂)ₙ—(CH₂—CH₂—O)ₙ₉CH₃, and n is 2 and n₉ is 6, 8, 12 or 24.

In some embodiments, when Q is O, then R^{Q} is a sugar moiety.

In some embodiments, when Q is O, then R^{Q} is

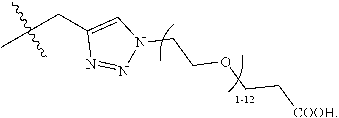

In some embodiments, when Q is O, then R^{Q}

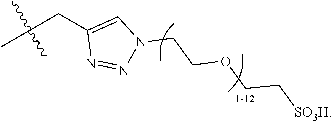

In some embodiments, when Q is O, then R^{Q} is

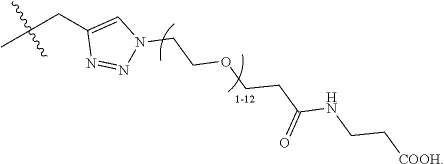

In some embodiments, when Q is O, then R^{Q} is

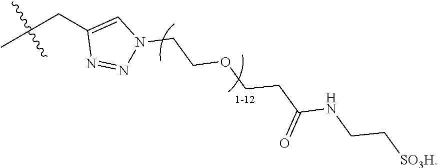

In some embodiments, when Q is O, then R^{Q} is

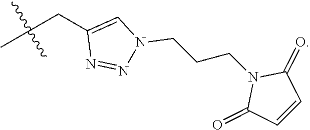 [SMILES: *Cc1cn(CCCN2C(=O)C=CC2=O)nn1]

In some embodiments, when Q is O, then R^{Q} is

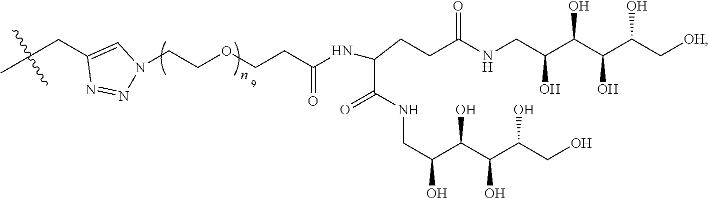

and n₉ is 6, 8, 12 or 24.

In some embodiments, when Q is O, then R^{Q} is

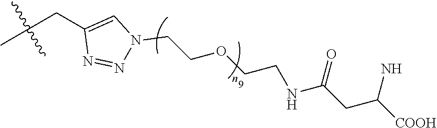

and n₉ is 6, 8, 12 or 24.

In some embodiments, the compound of Formula (I) contains at most one —SOₓM or —OSOₓM.

In some embodiments, R is —OSOₓM, —SOₓM, —OH, —OCH₃, O—(CH₂)₂—NH—C(O)—CH₂—O—CH₂—C(O)—NH—(CH₂—CH₂—O)₈CH₃.

In some embodiments,

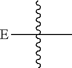

is

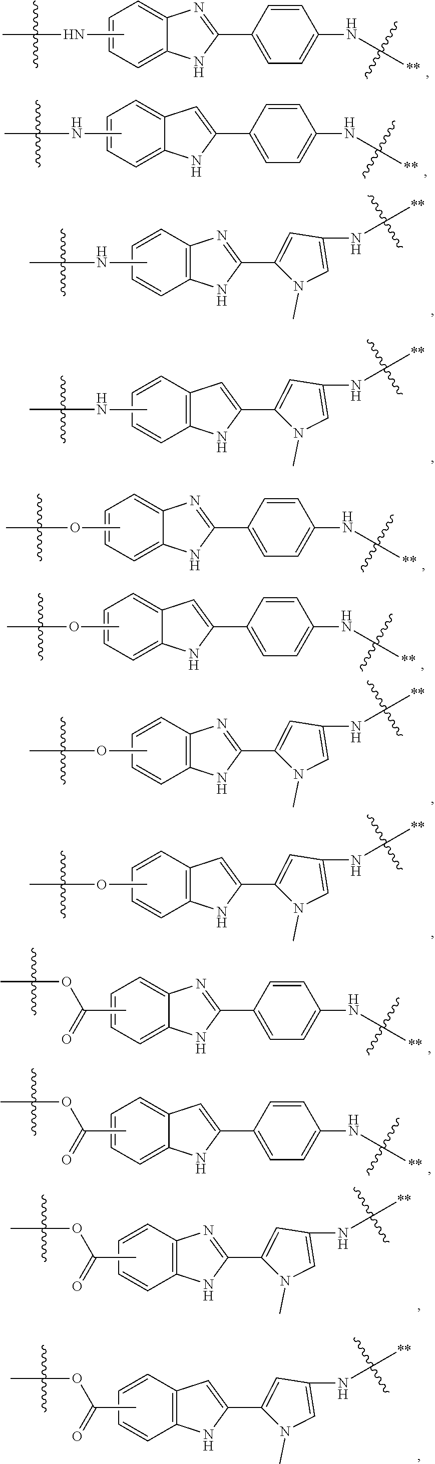

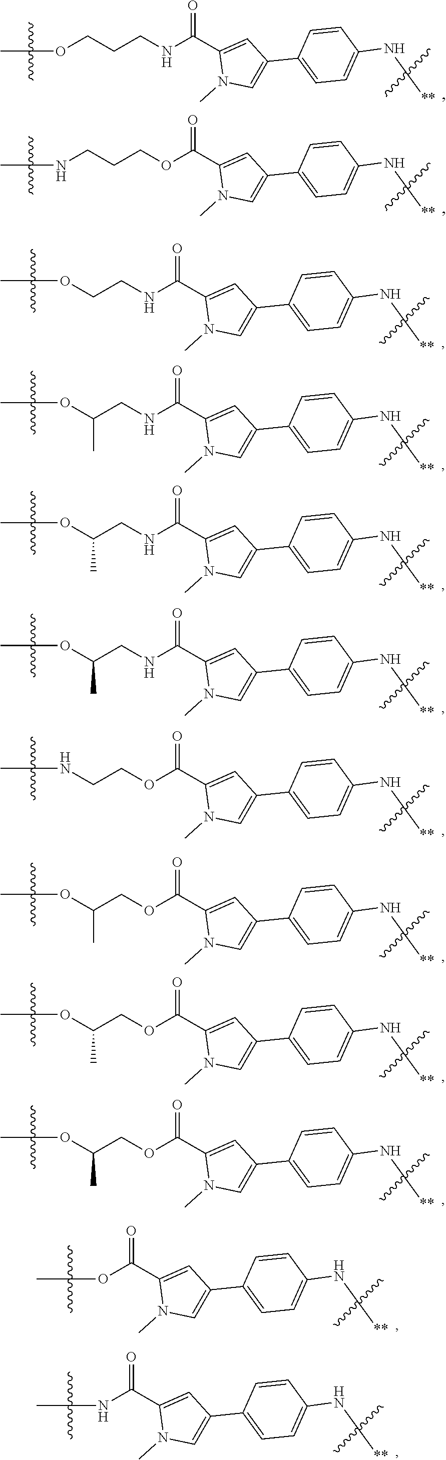

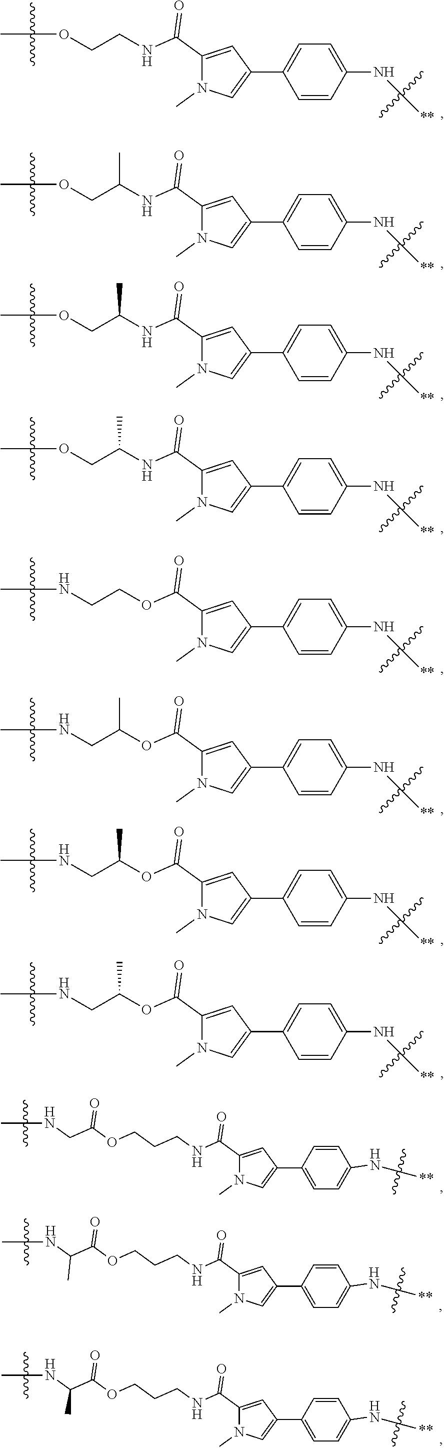

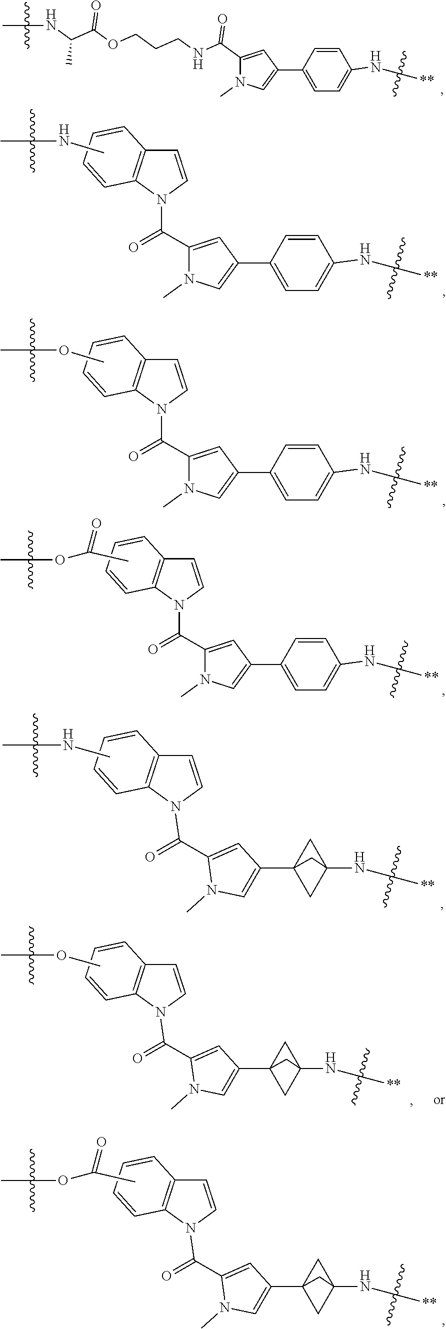

in which

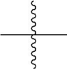

denotes a direct or indirect linkage to the PBRM, L^{C}, or L^{D}, and

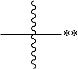

denotes a direct or indirect linkage to a remaining portion of D (e.g., a direct or indirect linkage to A).

In some embodiments,

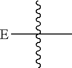

is

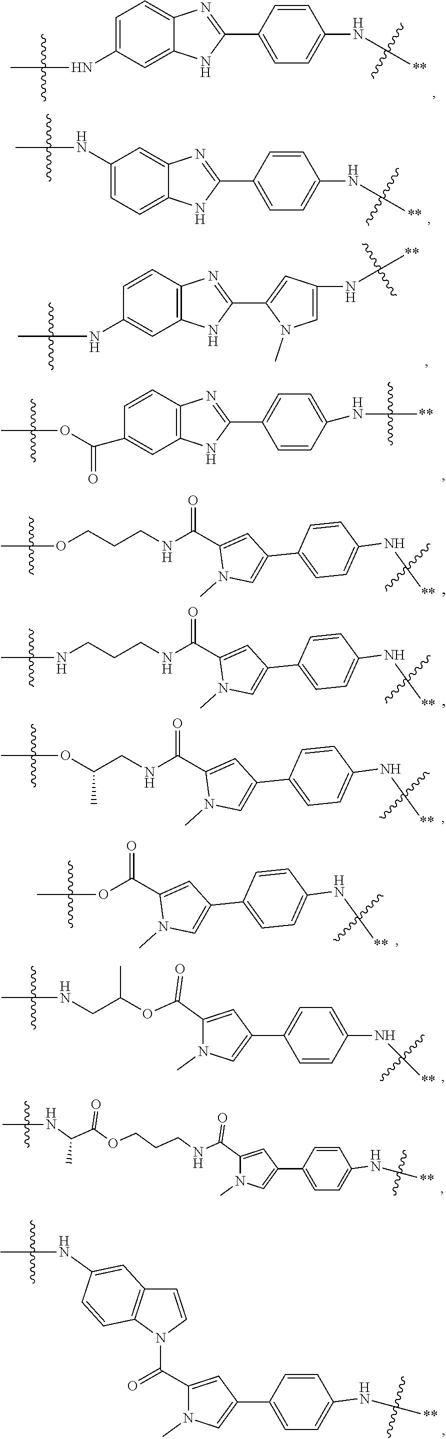

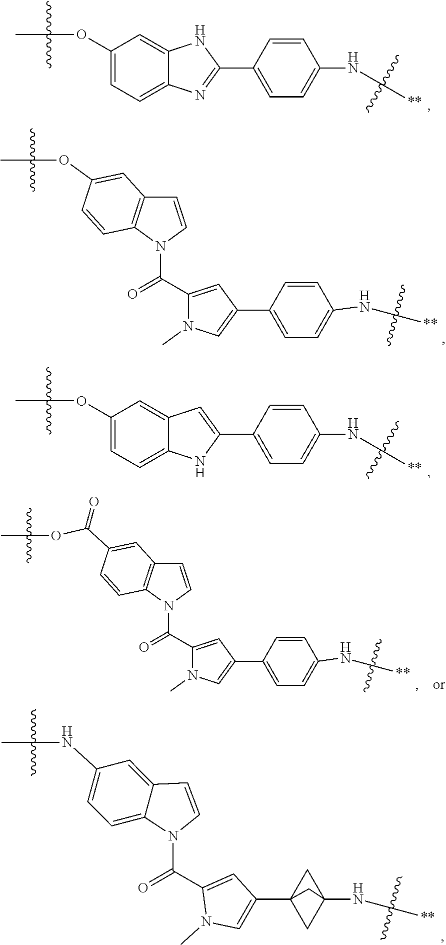

in which

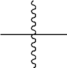

denotes a direct or indirect linkage to the PBRM, L^{C}, or L^{D}, and

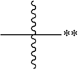

denotes a direct or indirect linkage to a remaining portion of D (e.g., a direct or a direct or indirect linkage to A).

In some embodiments,

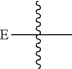

is

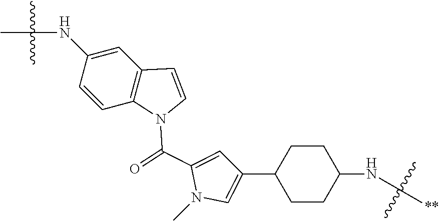

In some embodiments, E is

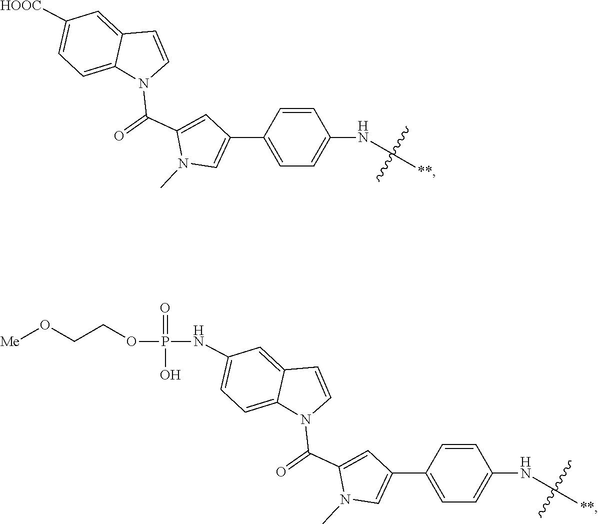

in which

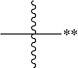

denotes a direct or indirect linkage to a remaining portion of D (e.g., a direct or indirect linkage to A)

In some embodiments, E is

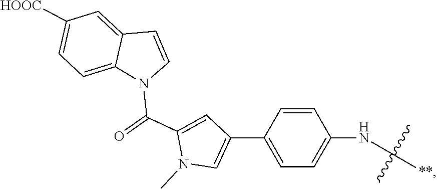

In some embodiments, E is

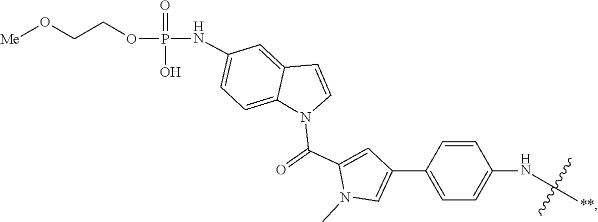

In some embodiments, the PBD drug moiety of Formula (IV) is of any one of Formulae (IX-a) to (IX-r):

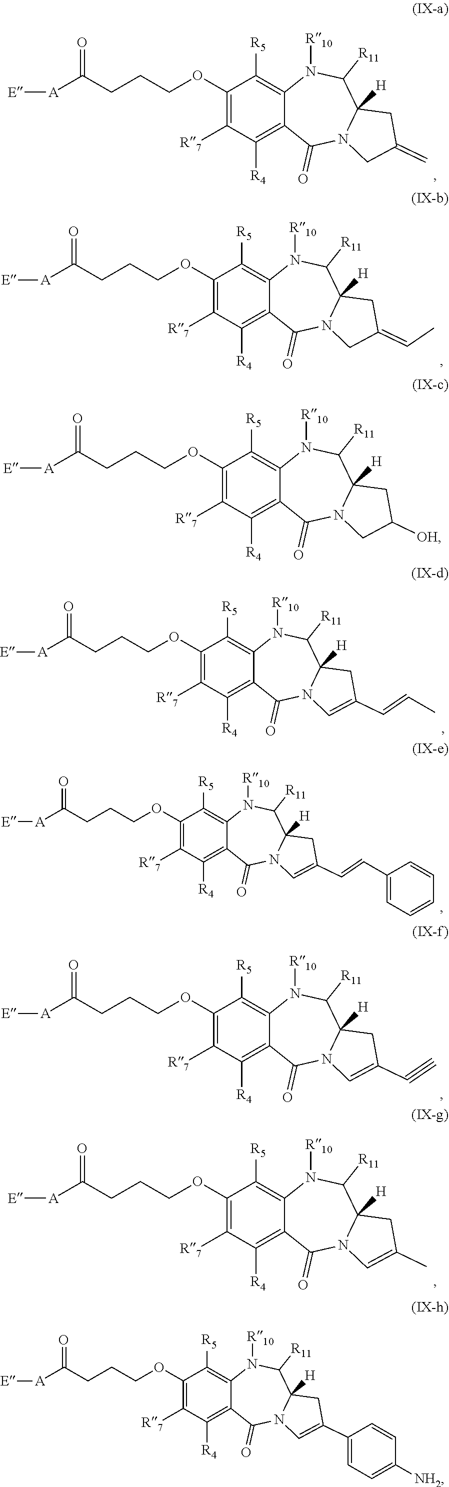

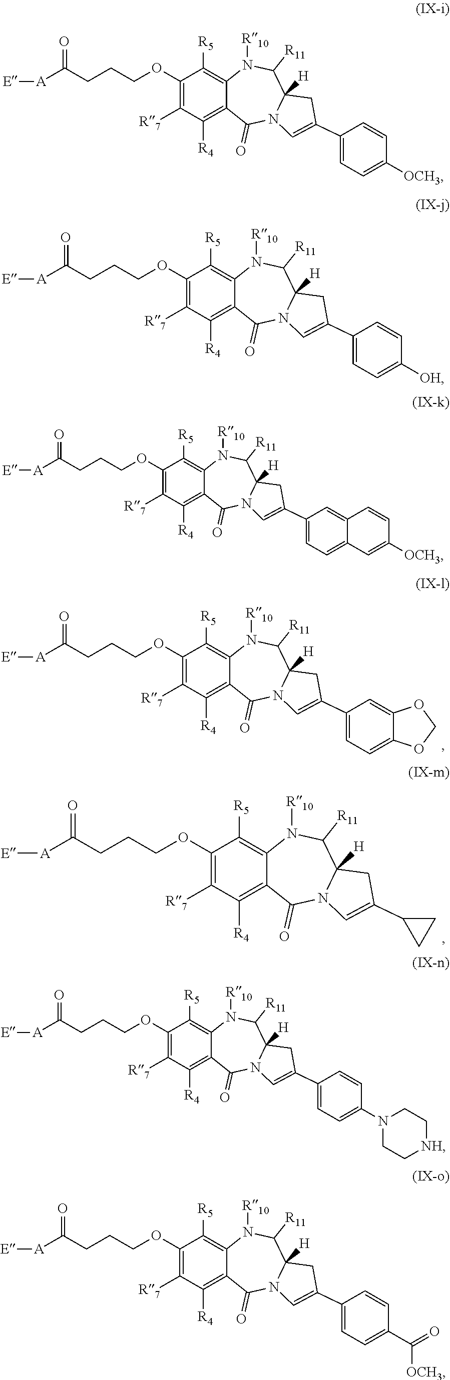

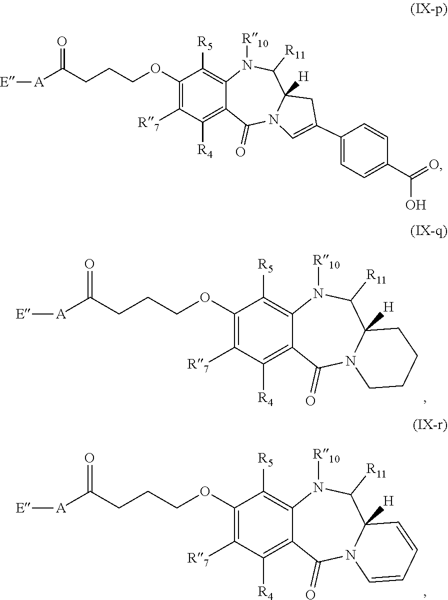

a tautomer thereof, a pharmaceutically acceptable salt or solvate thereof, or a pharmaceutically acceptable salt or solvate of the tautomer.

In some embodiments, the conjugates of any one of Formulae (IX-a)-(x-r) include those where each of the moieties defined for one of E″, A, R₄, R₅, R″₇, R″₁₀, and R₁₁ can be combined with any of the moieties defined for the others of E″, A, R₄, R₅, R″₇, R″₁₀, and R₁₁.

In some embodiments, the PBD drug moiety of Formula (IV) is of any one of Formulae (X-a) to (X-c):

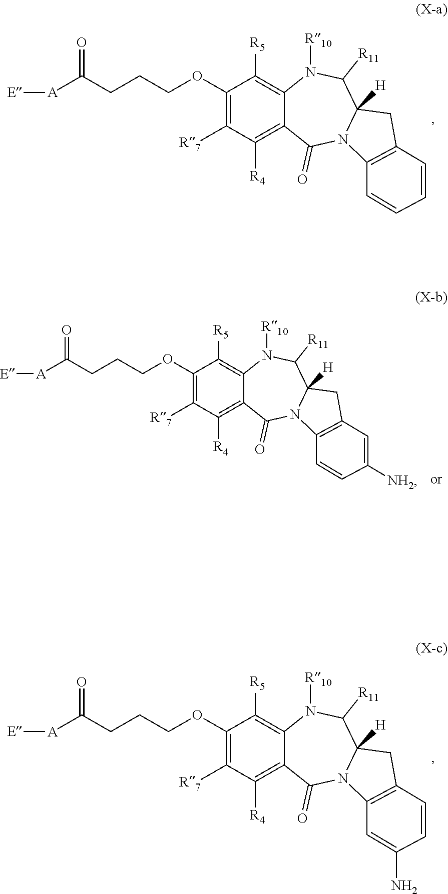

a tautomer thereof, a pharmaceutically acceptable salt or solvate thereof, or a pharmaceutically acceptable salt or solvate of the tautomer.

In some embodiments, the conjugates of any one of Formulae (X-a)-(X-c) include those where each of the moieties defined for one of E″, A, R₄, R₅, R″₇, R″₁₀, and R₁₁ can be combined with any of the moieties defined for the others of E″, A, R, R₁, R″₇, R″₁₀, and R₁₁.

In some embodiments, the PBD drug moiety of Formula (IV) is of any one of Formulae (XI-a) to (XI-c):

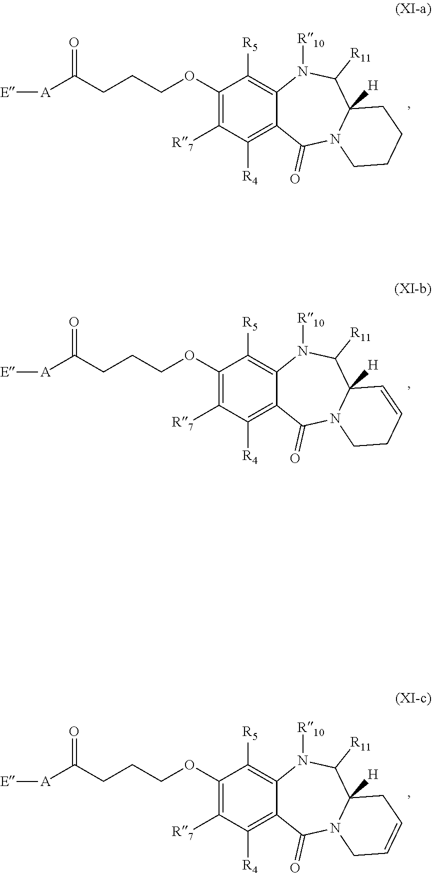

a tautomer thereof, a pharmaceutically acceptable salt or solvate thereof, or a pharmaceutically acceptable salt or solvate of the tautomer.

In some embodiments, the conjugates of any one of Formulae (XI-a)-(XI-c) include those where each of the moieties defined for one of E″, A, R, R₅, R″₇, R″₁₀, and R₁₁ can be combined with any of the moieties defined for the others of E″, A, R₄, R₅, R″₇, R″₁₀, and R₁₁.

In some embodiments, the PBD drug moiety of Formula (IV) is

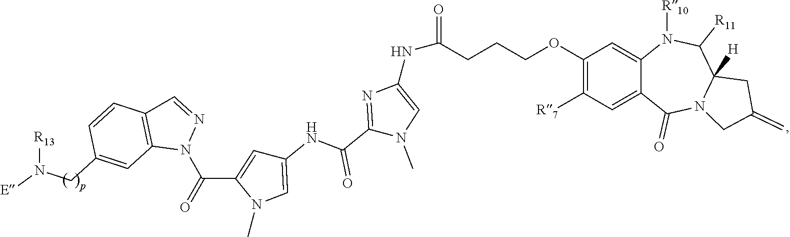

a tautomer thereof, a pharmaceutically acceptable salt or solvate thereof, or a pharmaceutically acceptable salt or solvate of the tautomer, wherein:

R₁₃ is H;

p is 1, 2, 3 or 4, and

E″, R″₇, R″₁₀ and R₁₁ are as defined herein.

In some embodiments, the PBD drug moiety of Formula (IV) is of Formula (XII):

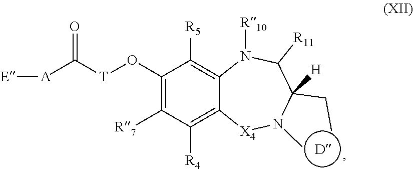

a tautomer thereof, a pharmaceutically acceptable salt or solvate thereof, or a pharmaceutically acceptable salt or solvate of the tautomer.

In some embodiments, the conjugates of Formula (XII) include those where each of the moieties defined for one of E″, A, T, R₄, R₅, R″₇, R″₁₀, R₁₁, X₄, and D″ can be combined with any of the moieties defined for the others of E″, A, T, R₄, R₅, R″₇, R″₁₀, R₁₁, X₄, and D″.

In the PBD drug moiety of Formula (XII) above, X₄ is C═S, CH₂, SO, SO₂ or BH; and E″, A, T, D″, R₄, R₅, R″₇R″₁₀ and R₁₁ are as defined herein.

In some embodiments, the PBD drug moiety of Formula (XII) is of any one of Formulae (XII-a) to (XII-e):

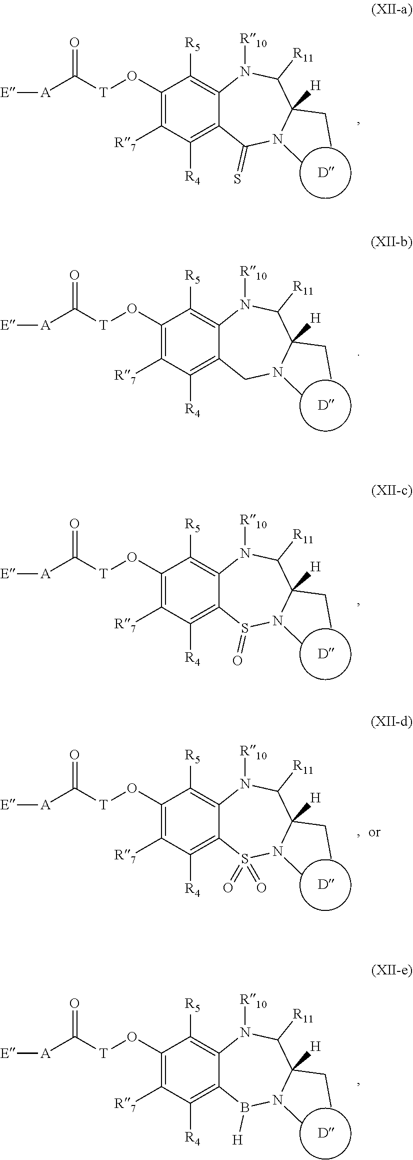

a tautomer thereof, a pharmaceutically acceptable salt or solvate thereof, or a pharmaceutically acceptable salt or solvate of the tautomer.

In some embodiments, the conjugates of any one of Formulae (XIIa)-(XIIe) include those where each of the moieties defined for one of E″, A, T, R₄, R₅, R″₇, R″₁₀, R₁₁, and D″ can be combined with any of the moieties defined for the others of E″, A, T, R₄, R₅, R″₇, R″₁₀, R₁₁, and D″.

In some embodiments, the PBD drug moiety (D), prior to being connected to another portion of the conjugate (e.g., the linker unit (L^{C})), corresponds to a compound selected from the compounds listed in Table 1, tautomers thereof, pharmaceutically acceptable salts or solvates thereof, or pharmaceutically acceptable salts or solvates of the tautomers.

TABLE 1
Structure

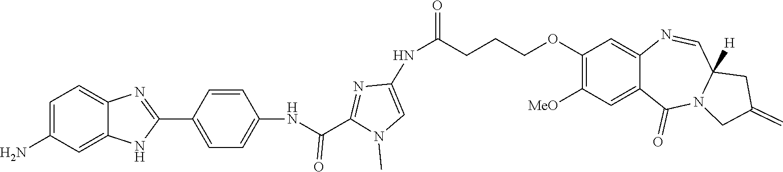 [SMILES: C=C1C[C@H]2C=Nc3cc(OCCCC(=O)Nc4cn(C)c(C(=O)Nc5ccc(-c6nc7ccc(N)cc7[nH]6)cc5)n4)c(OC)cc3C(=O)N2C1]

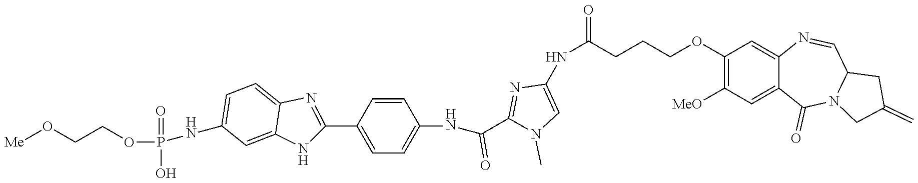 [SMILES: C=C1CC2C=Nc3cc(OCCCC(=O)Nc4cn(C)c(C(=O)Nc5ccc(-c6nc7ccc(NP(=O)(O)OCCOC)cc7[nH]6)cc5)n4)c(OC)cc3C(=O)N2C1]

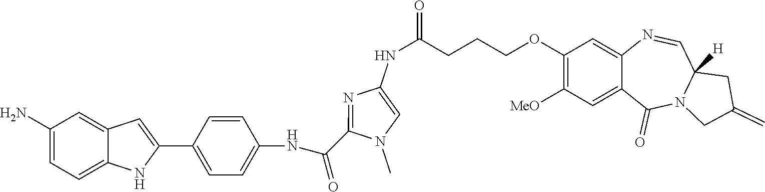 [SMILES: C=C1C[C@H]2C=Nc3cc(OCCCC(=O)Nc4cn(C)c(C(=O)Nc5ccc(-c6cc7cc(N)ccc7[nH]6)cc5)n4)c(OC)cc3C(=O)N2C1]

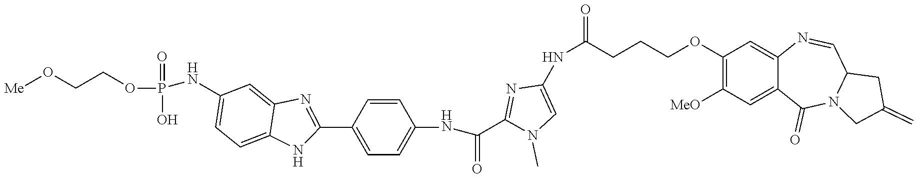 [SMILES: C=C1CC2C=Nc3cc(OCCCC(=O)Nc4cn(C)c(C(=O)Nc5ccc(-c6nc7cc(NP(=O)(O)OCCOC)ccc7[nH]6)cc5)n4)c(OC)cc3C(=O)N2C1]

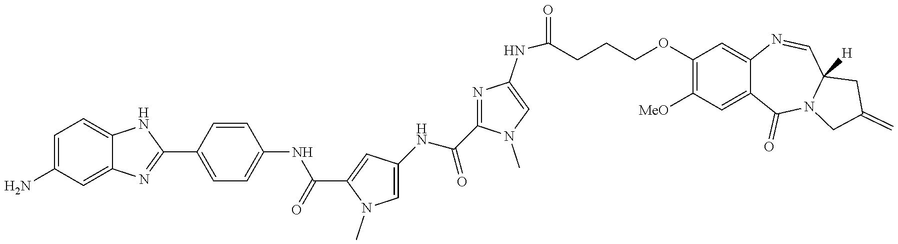 [SMILES: C=C1C[C@H]2C=Nc3cc(OCCCC(=O)Nc4cn(C)c(C(=O)Nc5cc(C(=O)Nc6ccc(-c7nc8cc(N)ccc8[nH]7)cc6)n(C)c5)n4)c(OC)cc3C(=O)N2C1]

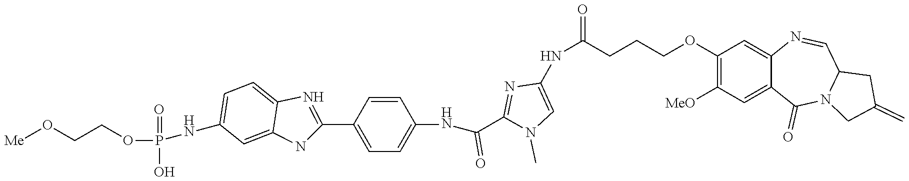 [SMILES: C=C1CC2C=Nc3cc(OCCCC(=O)Nc4cn(C)c(C(=O)Nc5ccc(-c6nc7ccc(NP(=O)(O)OCCOC)cc7[nH]6)cc5)n4)c(OC)cc3C(=O)N2C1]

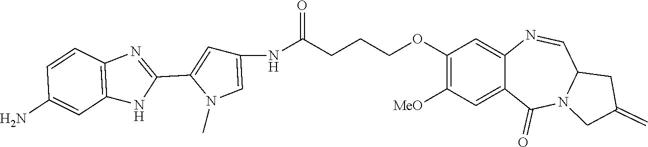 [SMILES: C=C1CC2C=Nc3cc(OCCCC(=O)Nc4cc(-c5nc6ccc(N)cc6[nH]5)n(C)c4)c(OC)cc3C(=O)N2C1]

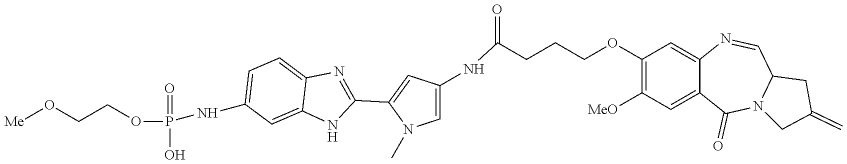 [SMILES: C=C1CC2C=Nc3cc(OCCCC(=O)Nc4cc(-c5nc6ccc(NP(=O)(O)OCCOC)cc6[nH]5)n(C)c4)c(OC)cc3C(=O)N2C1]

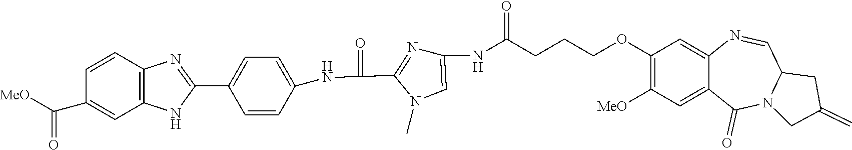 [SMILES: C=C1CC2C=Nc3cc(OCCCC(=O)Nc4cn(C)c(C(=O)Nc5ccc(-c6nc7ccc(C(=O)OC)cc7[nH]6)cc5)n4)c(OC)cc3C(=O)N2C1]

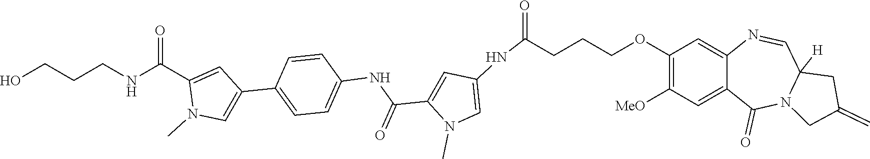 [SMILES: C=C1CC2C=Nc3cc(OCCCC(=O)Nc4cc(C(=O)Nc5ccc(-c6cc(C(=O)NCCCO)n(C)c6)cc5)n(C)c4)c(OC)cc3C(=O)N2C1]

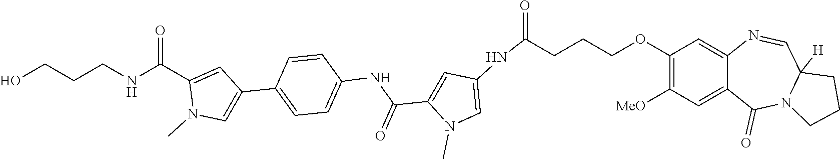 [SMILES: COc1cc2c(cc1OCCCC(=O)Nc1cc(C(=O)Nc3ccc(-c4cc(C(=O)NCCCO)n(C)c4)cc3)n(C)c1)N=CC1CCCN1C2=O]

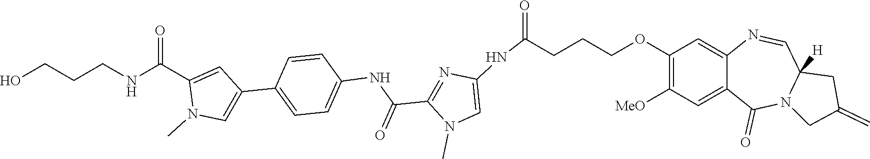 [SMILES: C=C1C[C@H]2C=Nc3cc(OCCCC(=O)Nc4cn(C)c(C(=O)Nc5ccc(-c6cc(C(=O)NCCCO)n(C)c6)cc5)n4)c(OC)cc3C(=O)N2C1]

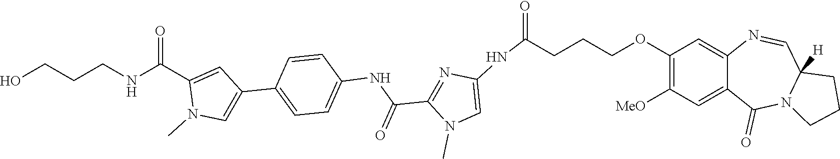 [SMILES: COc1cc2c(cc1OCCCC(=O)Nc1cn(C)c(C(=O)Nc3ccc(-c4cc(C(=O)NCCCO)n(C)c4)cc3)n1)N=C[C@@H]1CCCN1C2=O]

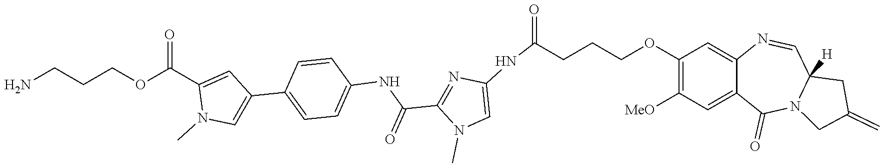 [SMILES: C=C1C[C@H]2C=Nc3cc(OCCCC(=O)Nc4cn(C)c(C(=O)Nc5ccc(-c6cc(C(=O)OCCCN)n(C)c6)cc5)n4)c(OC)cc3C(=O)N2C1]

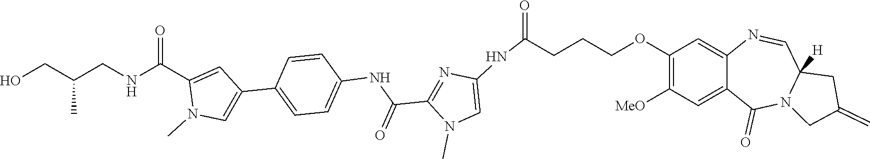 [SMILES: C=C1C[C@H]2C=Nc3cc(OCCCC(=O)Nc4cn(C)c(C(=O)Nc5ccc(-c6cc(C(=O)NC[C@H](C)CO)n(C)c6)cc5)n4)c(OC)cc3C(=O)N2C1]

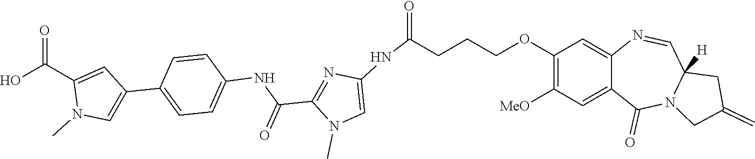 [SMILES: C=C1C[C@H]2C=Nc3cc(OCCCC(=O)Nc4cn(C)c(C(=O)Nc5ccc(-c6cc(C(=O)O)n(C)c6)cc5)n4)c(OC)cc3C(=O)N2C1]

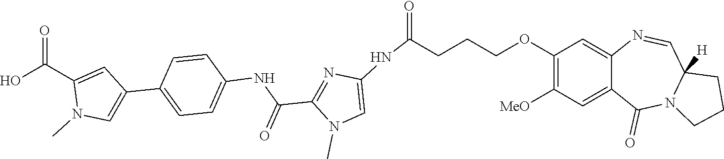 [SMILES: COc1cc2c(cc1OCCCC(=O)Nc1cn(C)c(C(=O)Nc3ccc(-c4cc(C(=O)O)n(C)c4)cc3)n1)N=C[C@@H]1CCCN1C2=O]

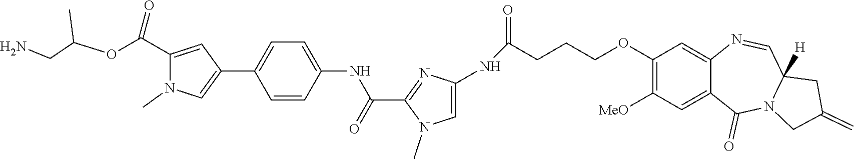 [SMILES: C=C1C[C@H]2C=Nc3cc(OCCCC(=O)Nc4cn(C)c(C(=O)Nc5ccc(-c6cc(C(=O)OC(C)CN)n(C)c6)cc5)n4)c(OC)cc3C(=O)N2C1]

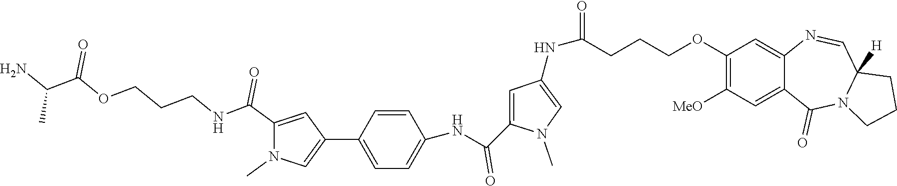 [SMILES: COc1cc2c(cc1OCCCC(=O)Nc1cc(C(=O)Nc3ccc(-c4cc(C(=O)NCCCOC(=O)[C@H](C)N)n(C)c4)cc3)n(C)c1)N=C[C@@H]1CCCN1C2=O]

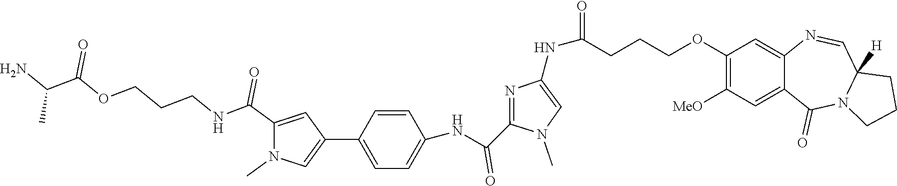 [SMILES: COc1cc2c(cc1OCCCC(=O)Nc1cn(C)c(C(=O)Nc3ccc(-c4cc(C(=O)NCCCOC(=O)[C@H](C)N)n(C)c4)cc3)n1)N=C[C@@H]1CCCN1C2=O]

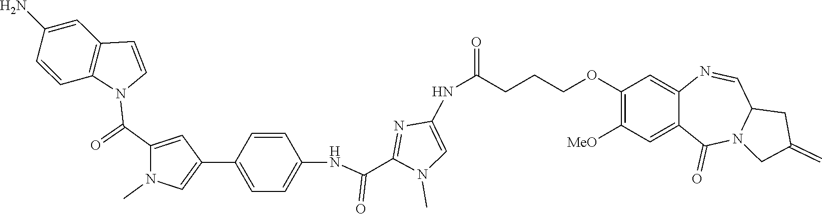 [SMILES: C=C1CC2C=Nc3cc(OCCCC(=O)Nc4cn(C)c(C(=O)Nc5ccc(-c6cc(C(=O)n7ccc8cc(N)ccc87)n(C)c6)cc5)n4)c(OC)cc3C(=O)N2C1]

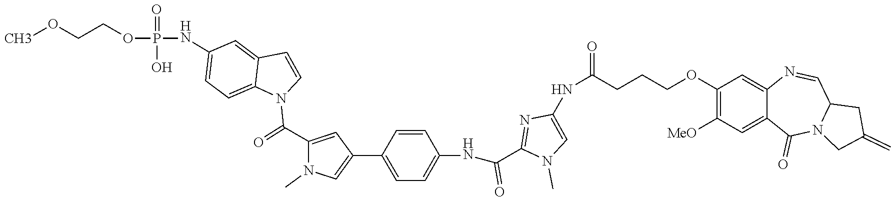

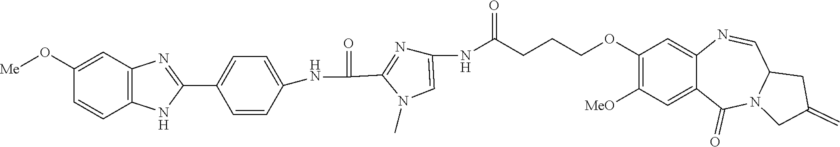 [SMILES: C=C1CC2C=Nc3cc(OCCCC(=O)Nc4cn(C)c(C(=O)Nc5ccc(-c6nc7cc(OC)ccc7[nH]6)cc5)n4)c(OC)cc3C(=O)N2C1]

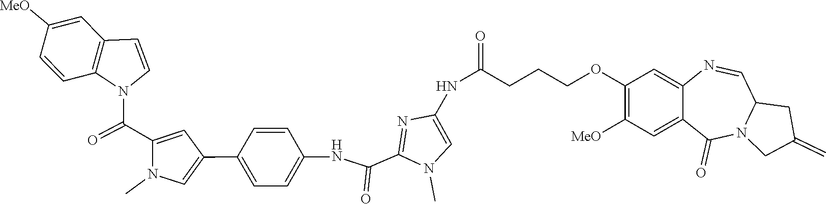 [SMILES: C=C1CC2C=Nc3cc(OCCCC(=O)Nc4cn(C)c(C(=O)Nc5ccc(-c6cc(C(=O)n7ccc8cc(OC)ccc87)n(C)c6)cc5)n4)c(OC)cc3C(=O)N2C1]

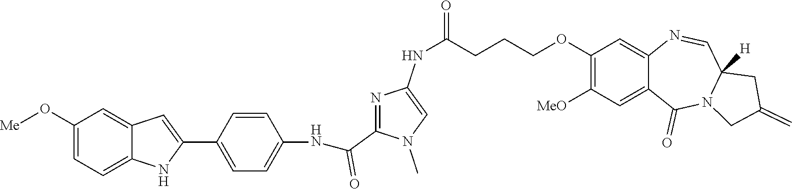 [SMILES: C=C1C[C@H]2C=Nc3cc(OCCCC(=O)Nc4cn(C)c(C(=O)Nc5ccc(-c6cc7cc(OC)ccc7[nH]6)cc5)n4)c(OC)cc3C(=O)N2C1]

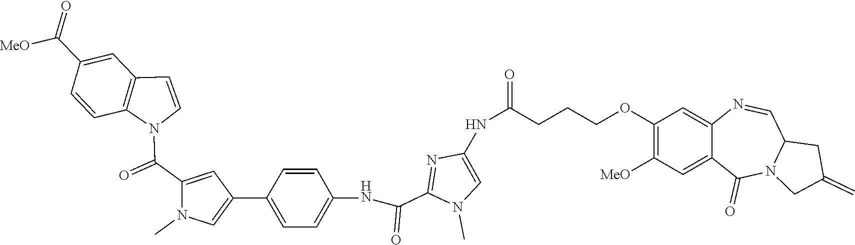 [SMILES: C=C1CC2C=Nc3cc(OCCCC(=O)Nc4cn(C)c(C(=O)Nc5ccc(-c6cc(C(=O)n7ccc8cc(C(=O)OC)ccc87)n(C)c6)cc5)n4)c(OC)cc3C(=O)N2C1]

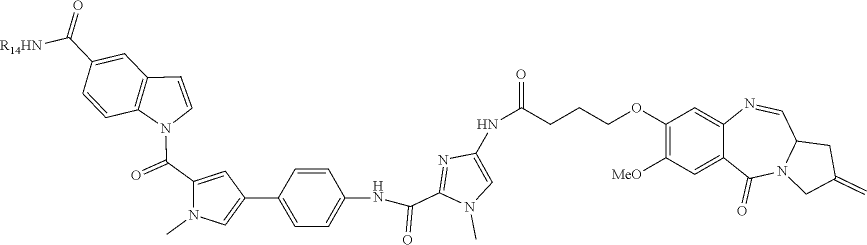

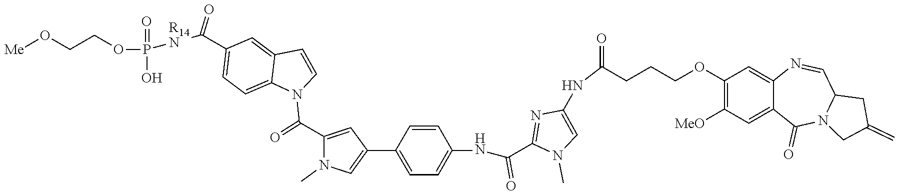

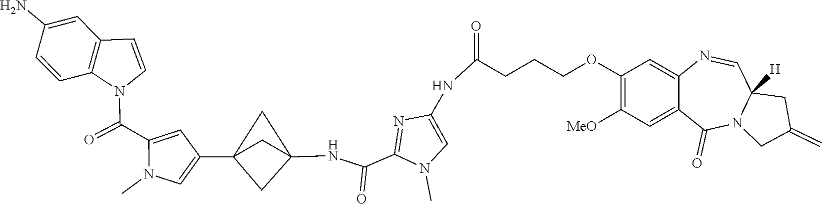 [SMILES: C=C1C[C@H]2C=Nc3cc(OCCCC(=O)Nc4cn(C)c(C(=O)NC56CC(c7cc(C(=O)n8ccc9cc(N)ccc98)n(C)c7)(C5)C6)n4)c(OC)cc3C(=O)N2C1]

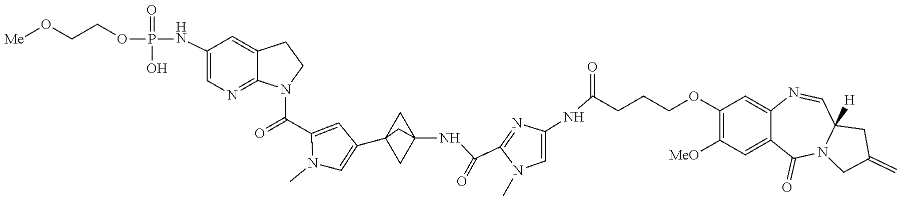 [SMILES: C=C1C[C@H]2C=Nc3cc(OCCCC(=O)Nc4cn(C)c(C(=O)NC56CC(c7cc(C(=O)N8CCc9cc(NP(=O)(O)OCCOC)cnc98)n(C)c7)(C5)C6)n4)c(OC)cc3C(=O)N2C1]

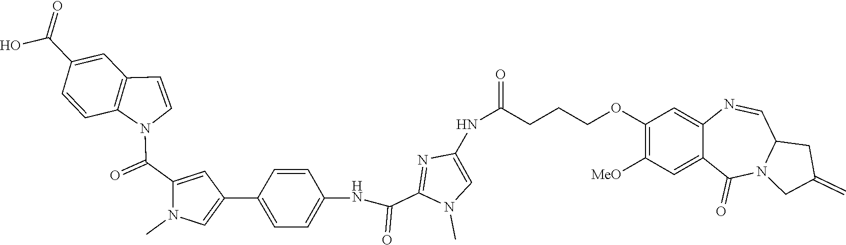 [SMILES: C=C1CC2C=Nc3cc(OCCCC(=O)Nc4cn(C)c(C(=O)Nc5ccc(-c6cc(C(=O)n7ccc8cc(C(=O)O)ccc87)n(C)c6)cc5)n4)c(OC)cc3C(=O)N2C1]

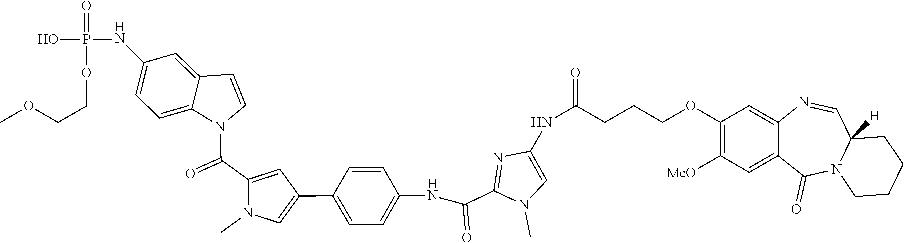 [SMILES: COCCOP(=O)(O)Nc1ccc2c(ccn2C(=O)c2cc(-c3ccc(NC(=O)c4nc(NC(=O)CCCOc5cc6c(cc5OC)C(=O)N5CCCC[C@H]5C=N6)cn4C)cc3)cn2C)c1]

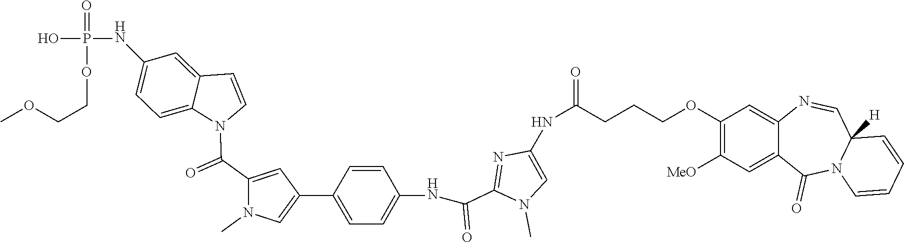 [SMILES: COCCOP(=O)(O)Nc1ccc2c(ccn2C(=O)c2cc(-c3ccc(NC(=O)c4nc(NC(=O)CCCOc5cc6c(cc5OC)C(=O)N5C=CC=C[C@H]5C=N6)cn4C)cc3)cn2C)c1]

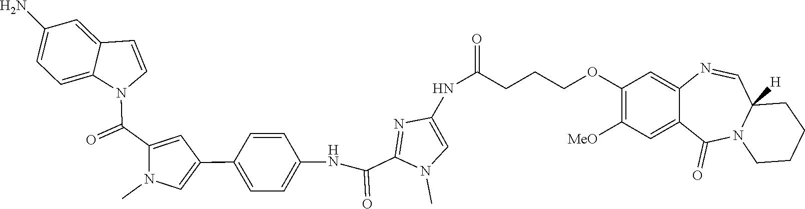 [SMILES: COc1cc2c(cc1OCCCC(=O)Nc1cn(C)c(C(=O)Nc3ccc(-c4cc(C(=O)n5ccc6cc(N)ccc65)n(C)c4)cc3)n1)N=C[C@@H]1CCCCN1C2=O]

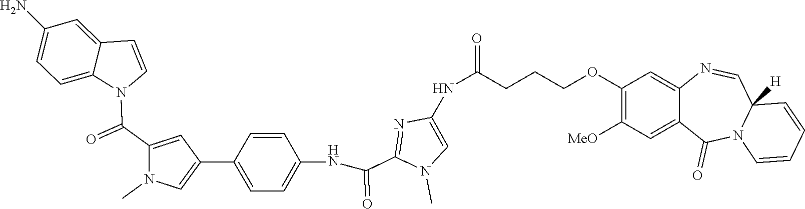 [SMILES: COc1cc2c(cc1OCCCC(=O)Nc1cn(C)c(C(=O)Nc3ccc(-c4cc(C(=O)n5ccc6cc(N)ccc65)n(C)c4)cc3)n1)N=C[C@@H]1C=CC=CN1C2=O]

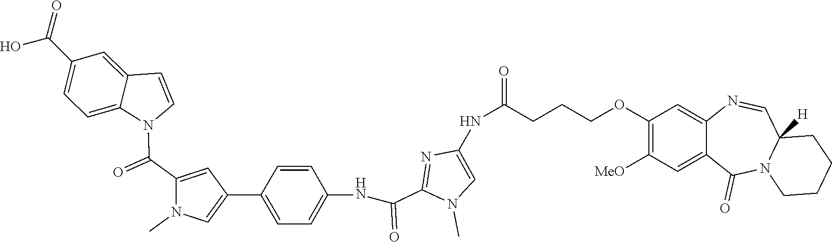 [SMILES: COc1cc2c(cc1OCCCC(=O)Nc1cn(C)c(C(=O)Nc3ccc(-c4cc(C(=O)n5ccc6cc(C(=O)O)ccc65)n(C)c4)cc3)n1)N=C[C@@H]1CCCCN1C2=O]

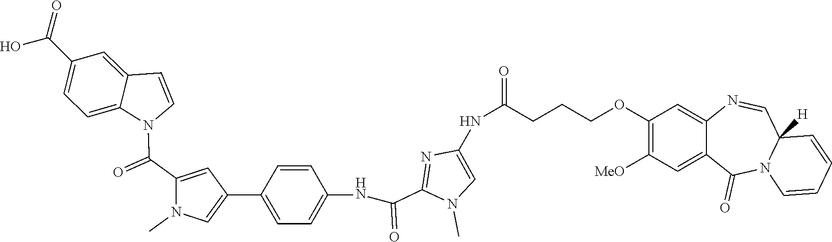 [SMILES: COc1cc2c(cc1OCCCC(=O)Nc1cn(C)c(C(=O)Nc3ccc(-c4cc(C(=O)n5ccc6cc(C(=O)O)ccc65)n(C)c4)cc3)n1)N=C[C@@H]1C=CC=CN1C2=O]

In some embodiments, the PBD drug moiety (D), prior to being connected to another portion of the conjugate (e.g., the linker unit (L^{C})), corresponds to a compound of any one of Formula (XIIIa) to (XIIIm):

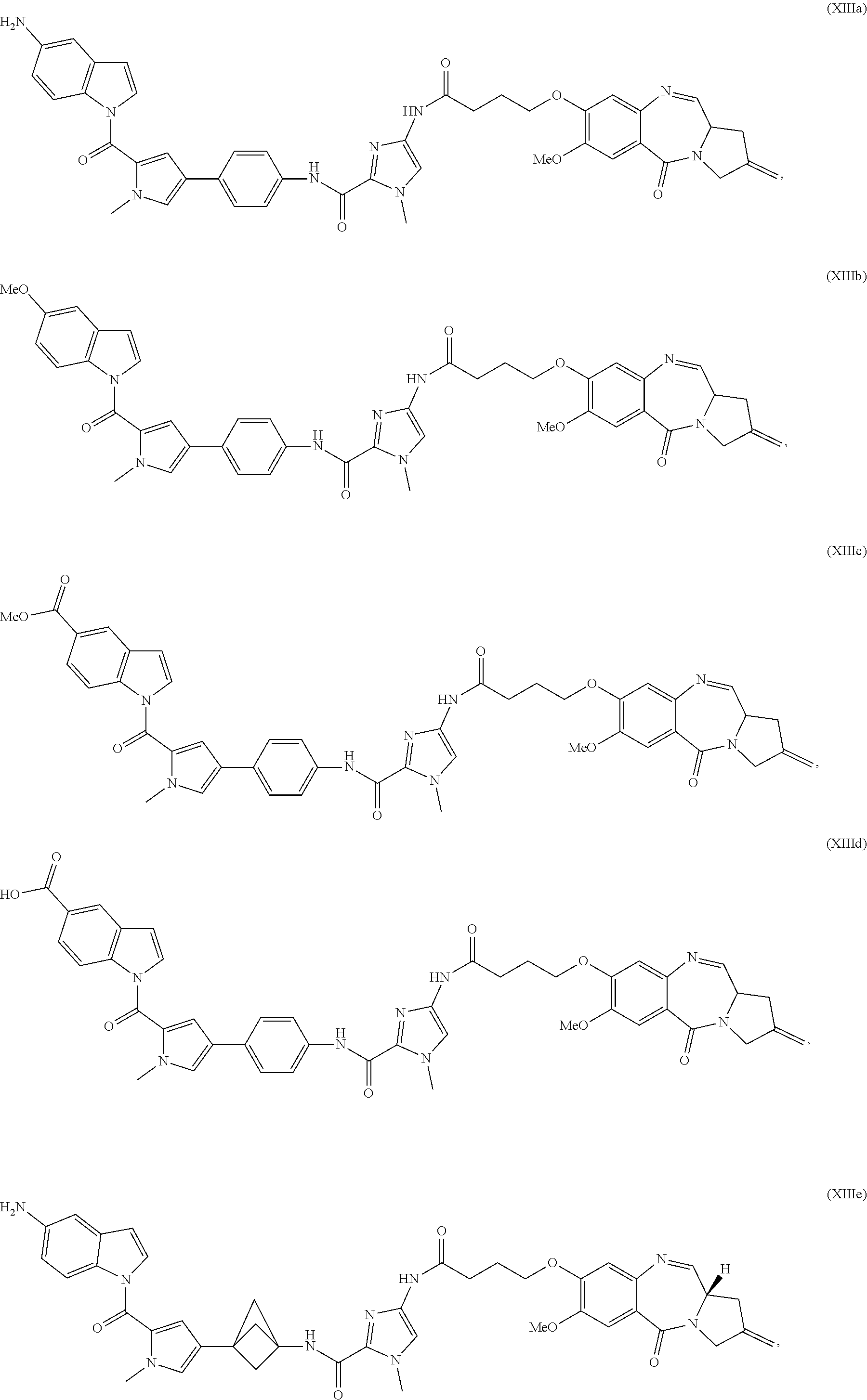

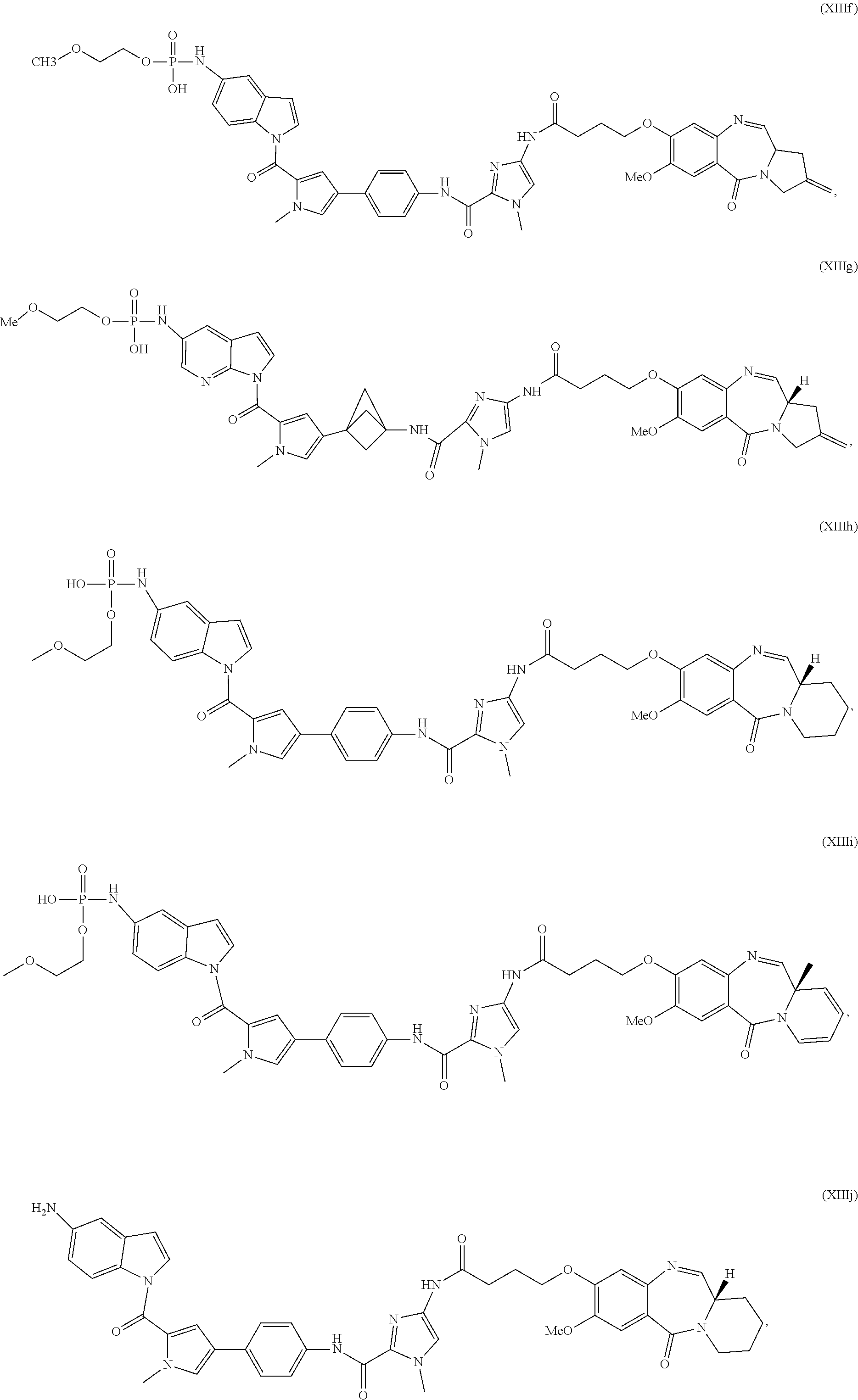

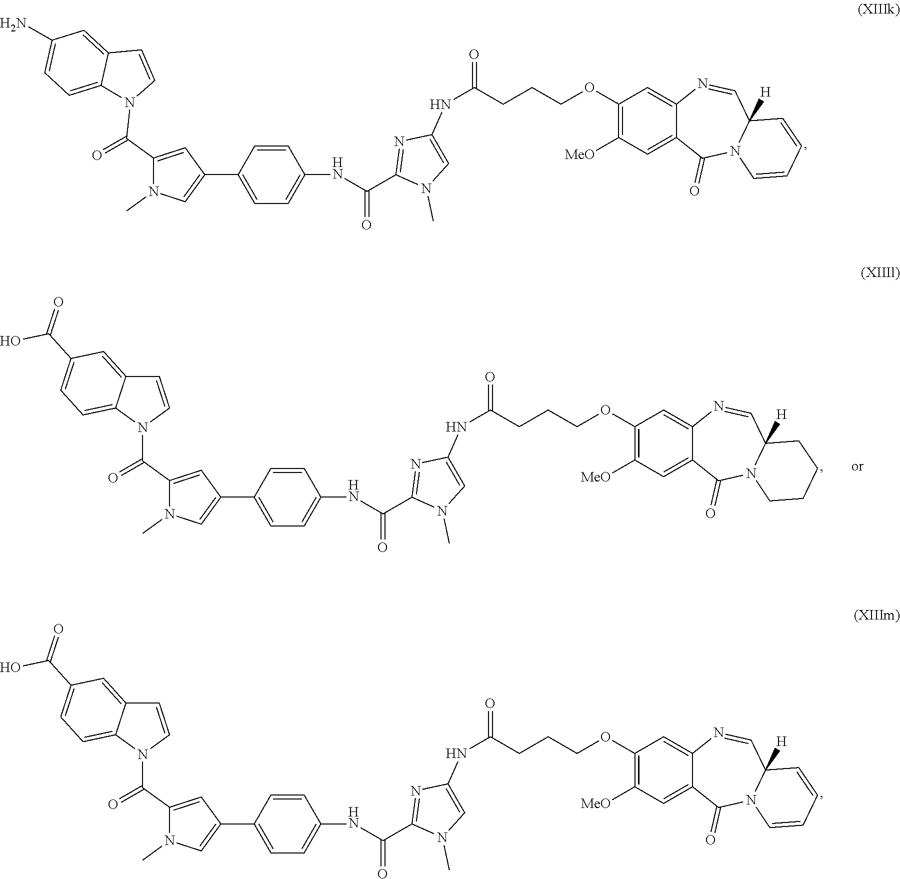 [SMILES: COc1cc2c(cc1OCCCC(=O)Nc1cn(C)c(C(=O)Nc3ccc(-c4cc(C(=O)n5ccc6cc(N)ccc65)n(C)c4)cc3)n1)N=C[C@@H]1C=CC=CN1C2=O ; COc1cc2c(cc1OCCCC(=O)Nc1cn(C)c(C(=O)Nc3ccc(-c4cc(C(=O)n5ccc6cc(C(=O)O)ccc65)n(C)c4)cc3)n1)N=C[C@@H]1CCCCN1C2=O ; COc1cc2c(cc1OCCCC(=O)Nc1cn(C)c(C(=O)Nc3ccc(-c4cc(C(=O)n5ccc6cc(C(=O)O)ccc65)n(C)c4)cc3)n1)N=C[C@@H]1C=CC=CN1C2=O]

a tautomer thereof, a pharmaceutically acceptable salt or solvate thereof, or a pharmaceutically acceptable salt or solvate of the tautomer.

In some embodiments, the PBD drug moiety (D), connected to another portion of the conjugate (e.g., the linker unit (L^{C})), corresponds to a conjugate selected from the conjugates listed in Table 1A, tautomers thereof, pharmaceutically acceptable salts or solvates thereof, or pharmaceutically acceptable salts or solvates of the tautomers, wherein

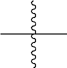

indicates the point of attachment to the linker unit.

TABLE 1A

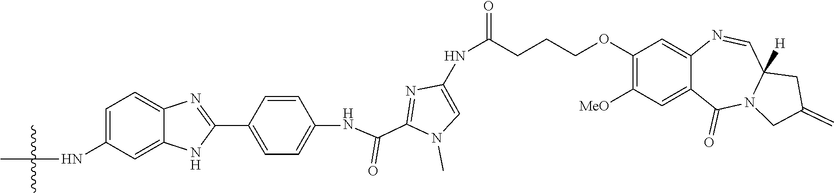

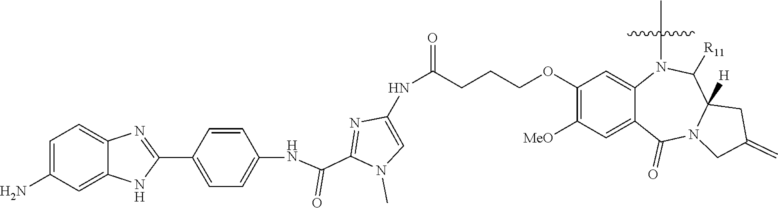

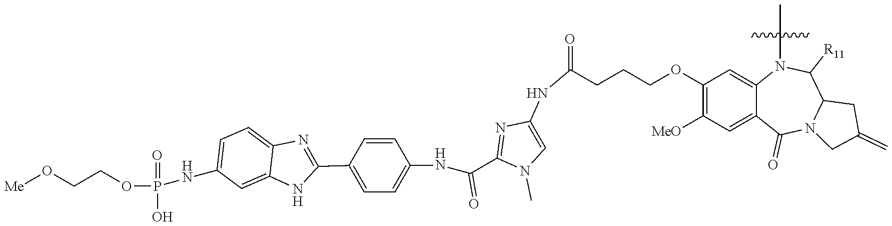

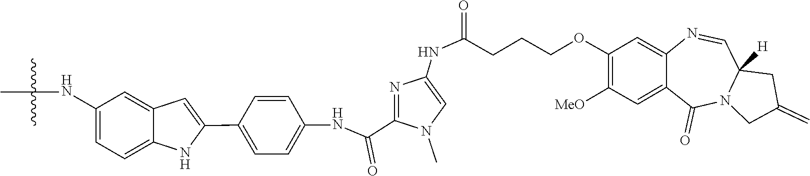

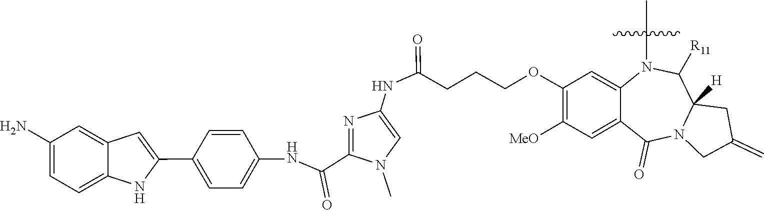

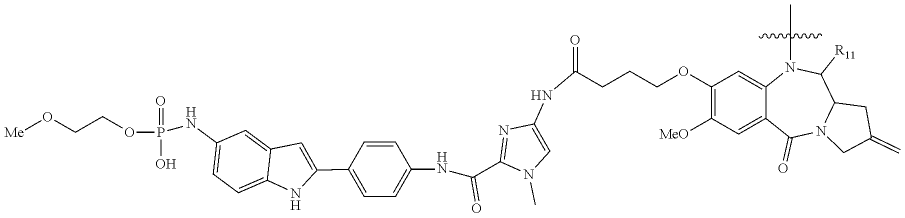

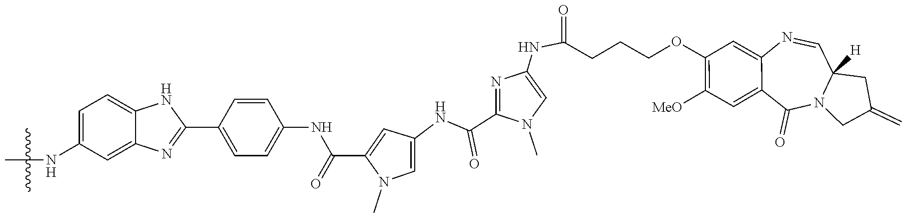

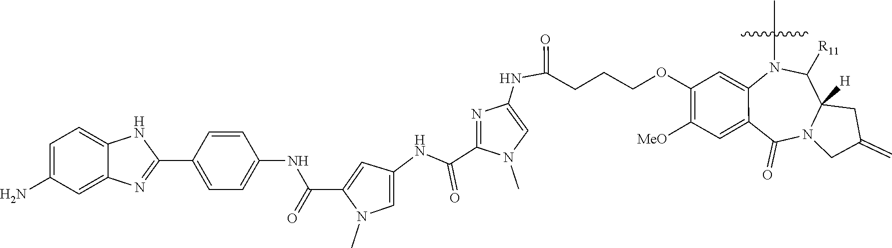

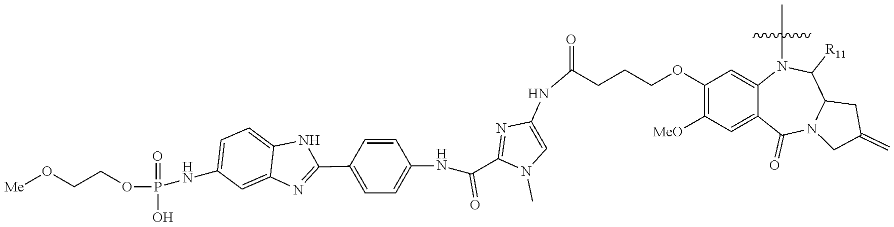

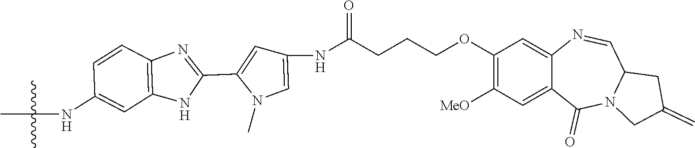

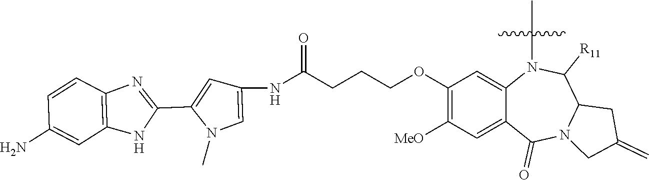

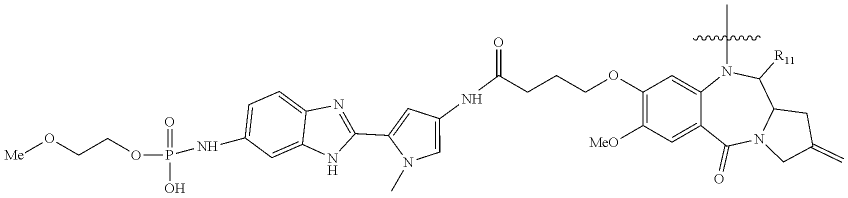

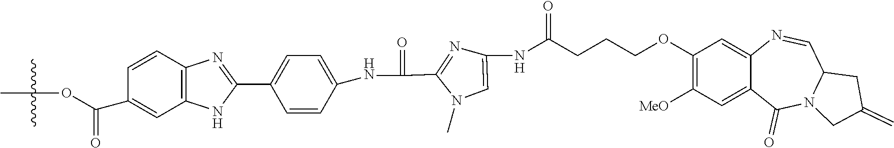

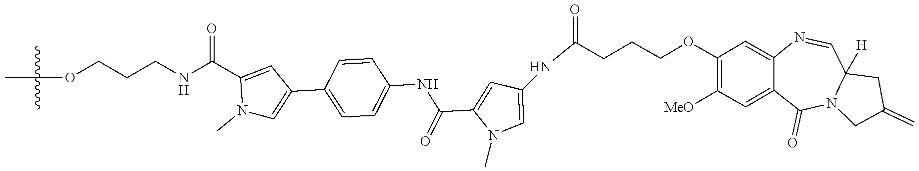

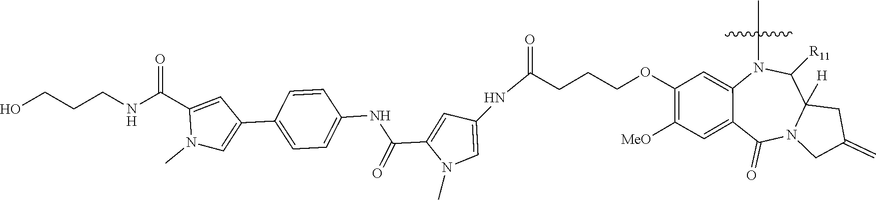

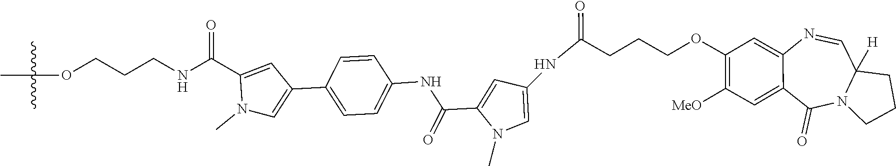

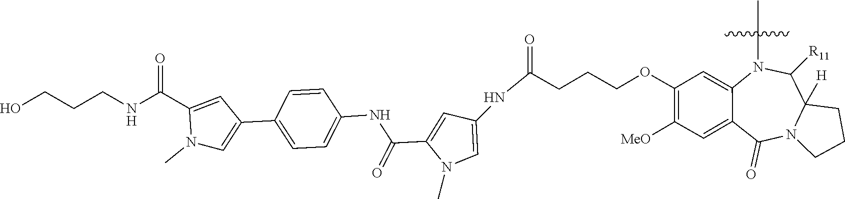

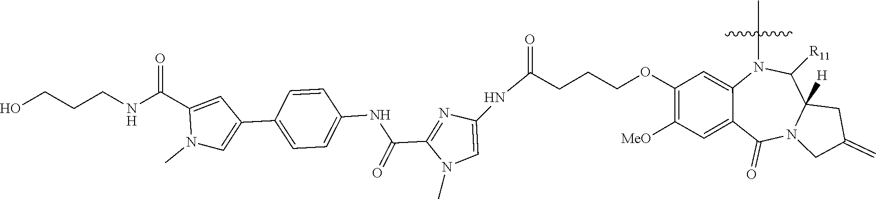

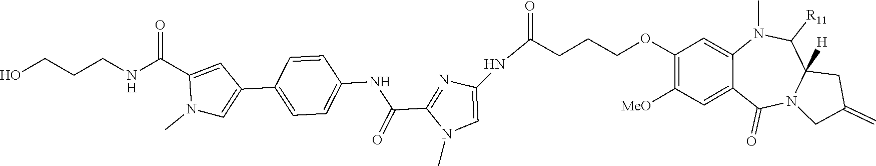

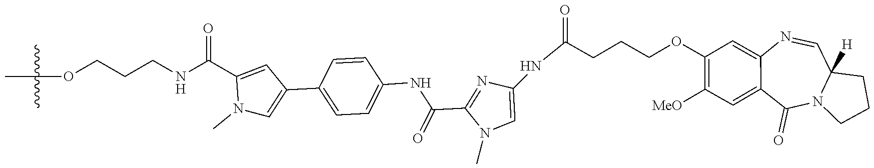

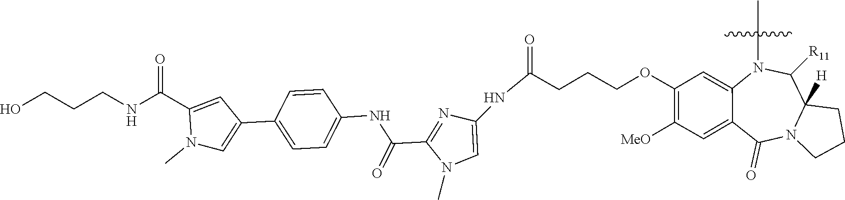

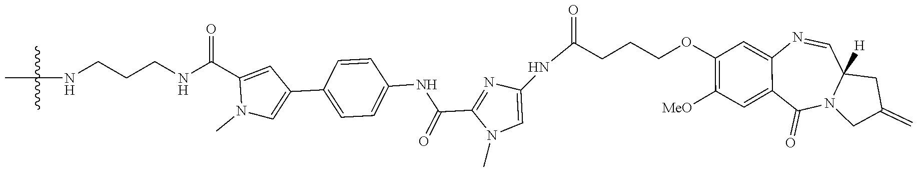

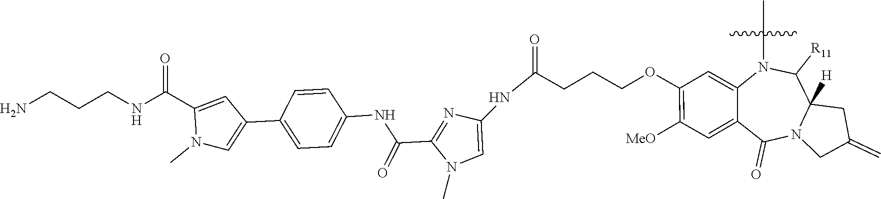

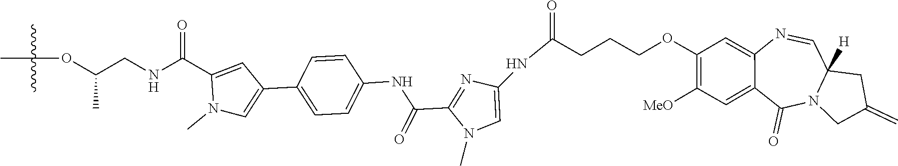

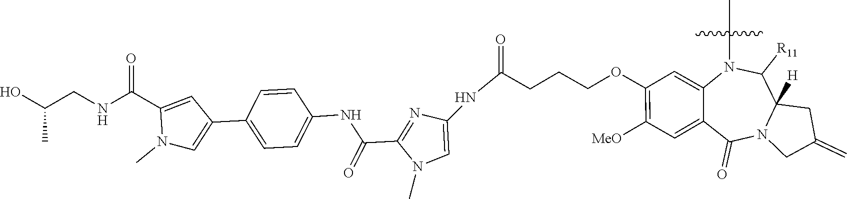

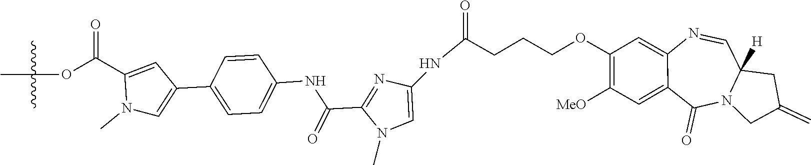

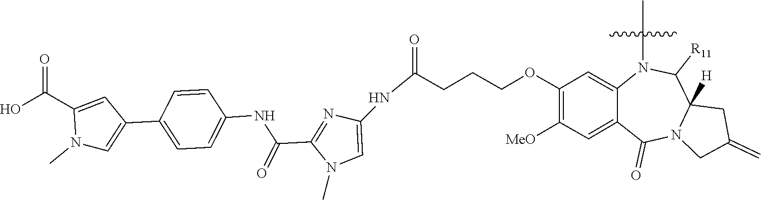

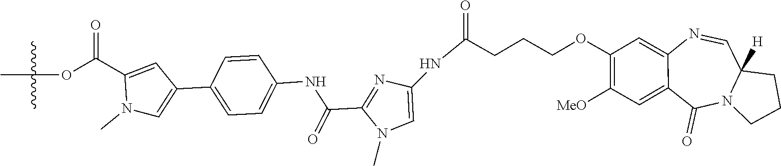

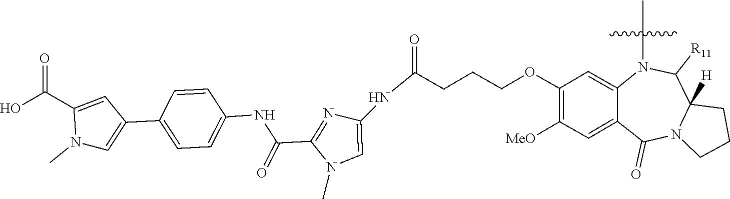

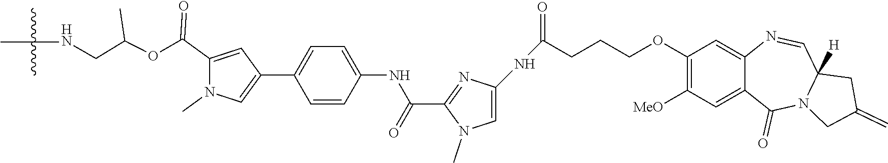

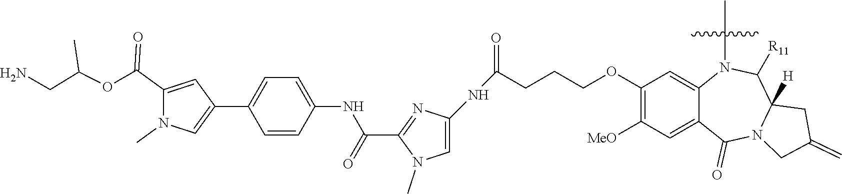

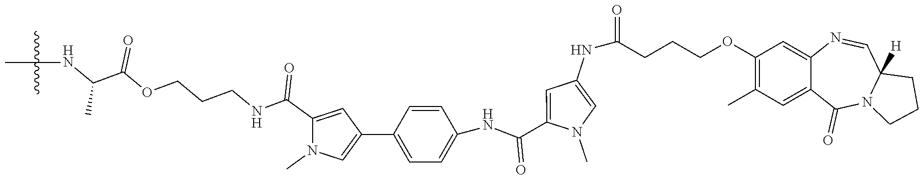

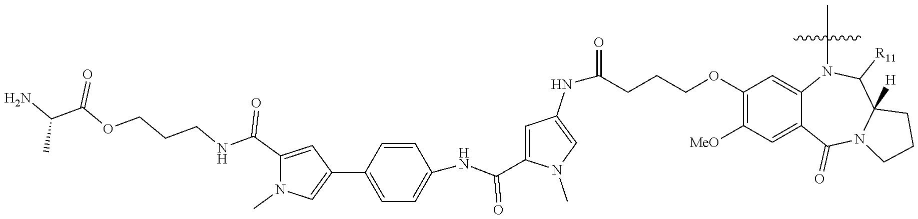

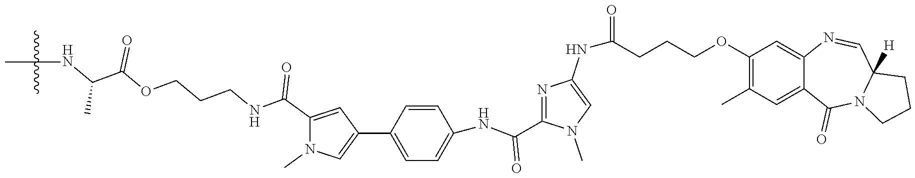

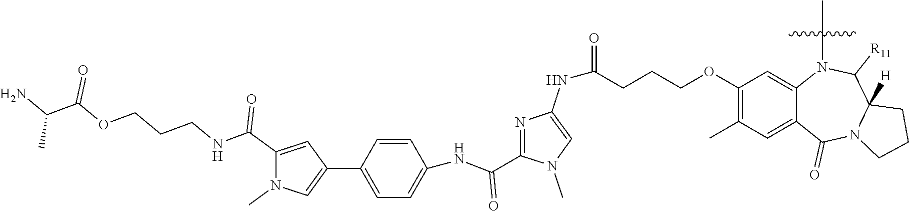

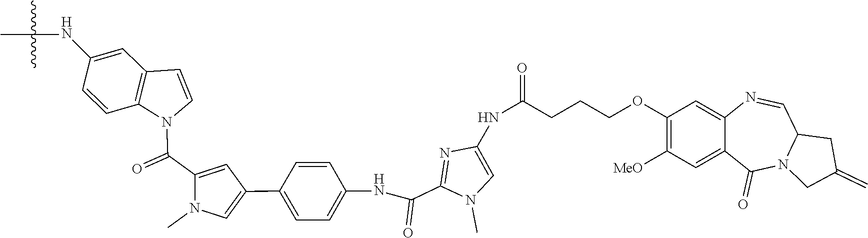

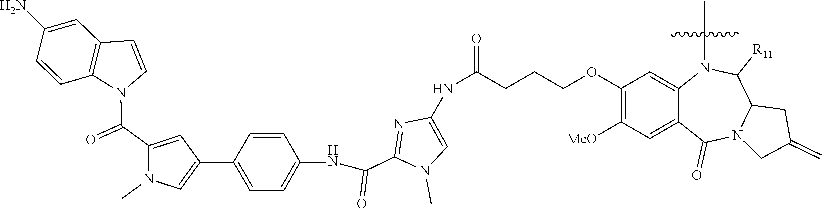

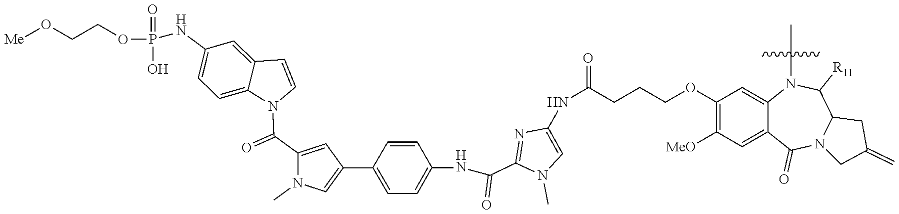

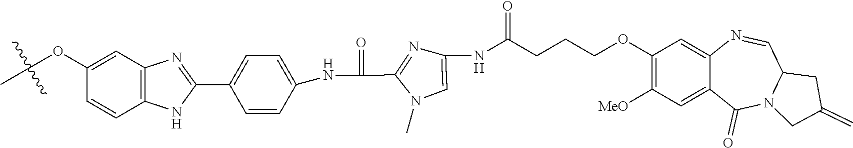

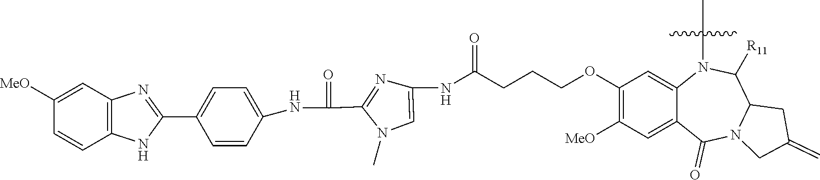

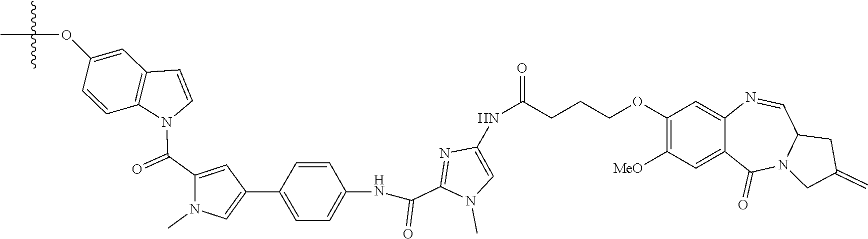

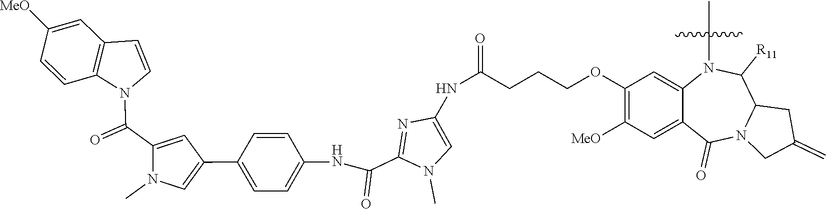

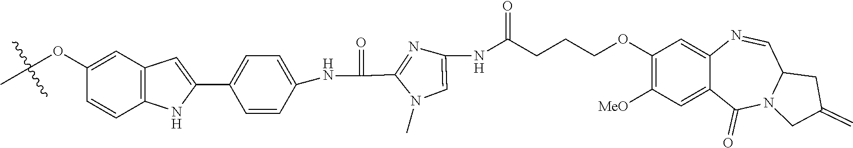

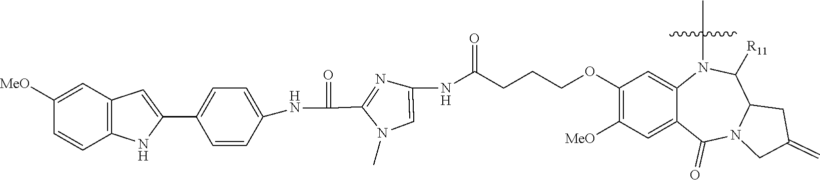

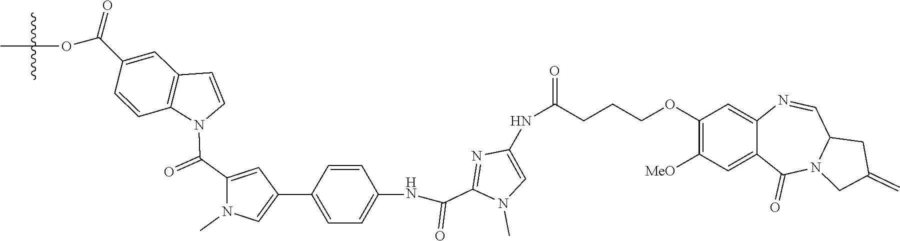

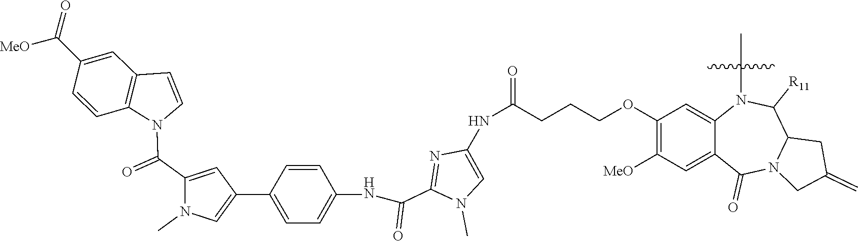

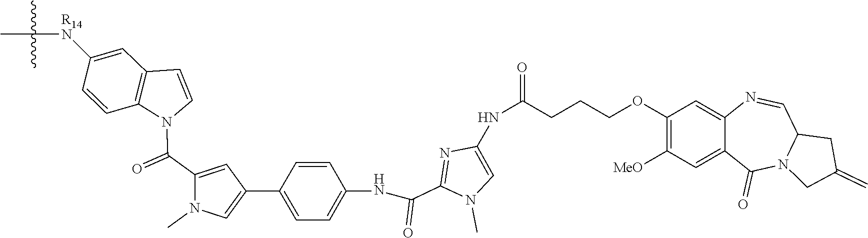

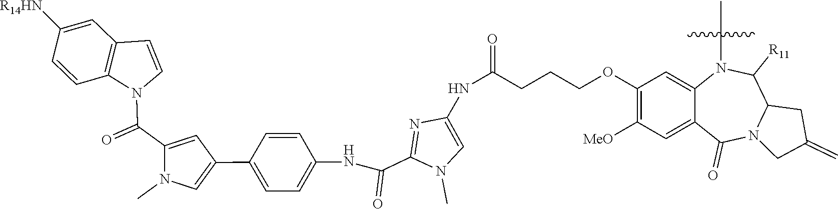

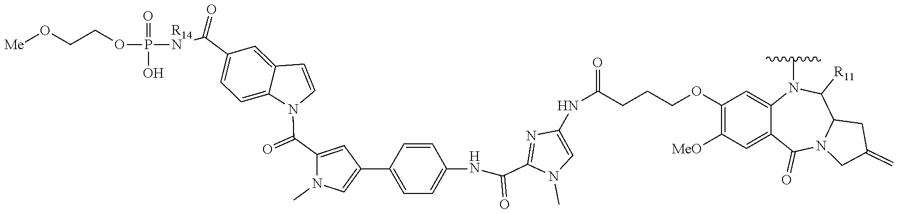

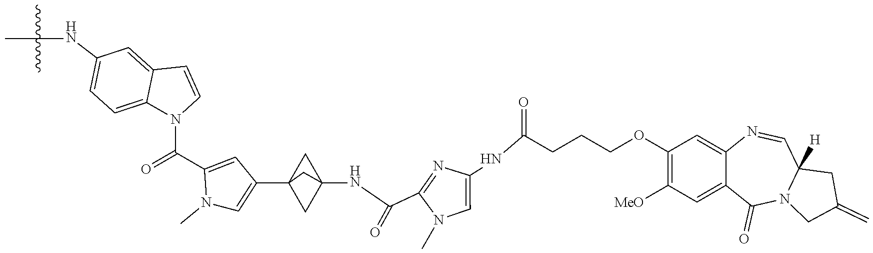

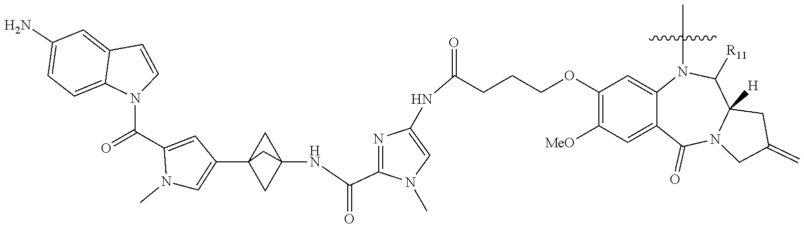

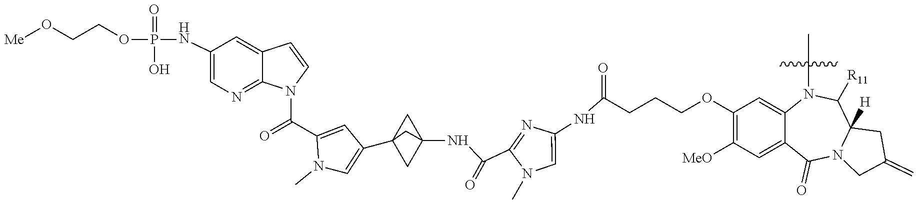

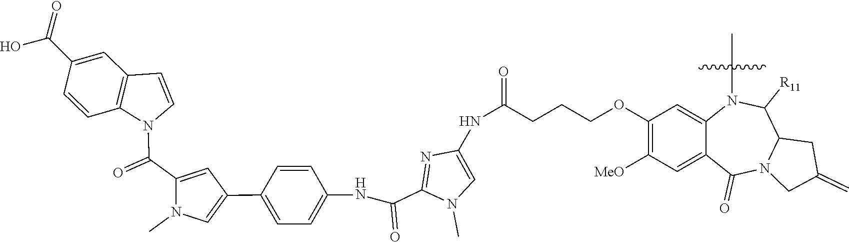

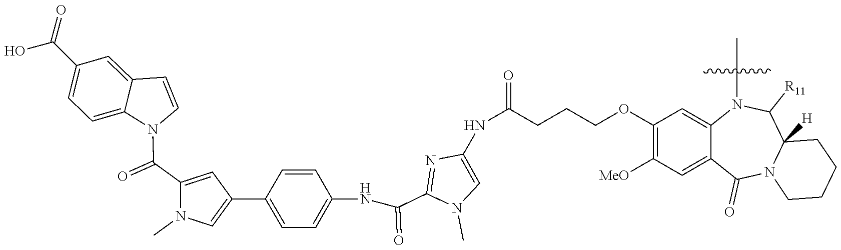

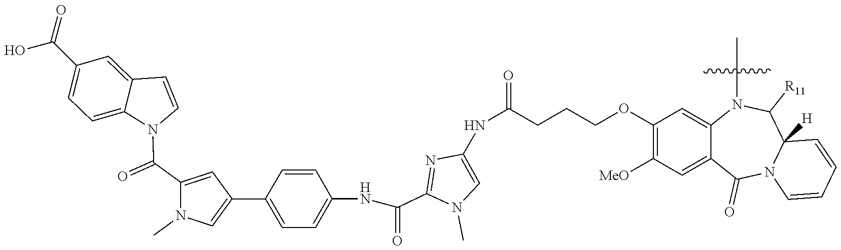

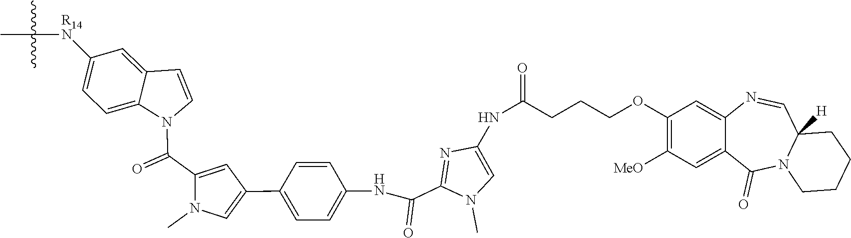

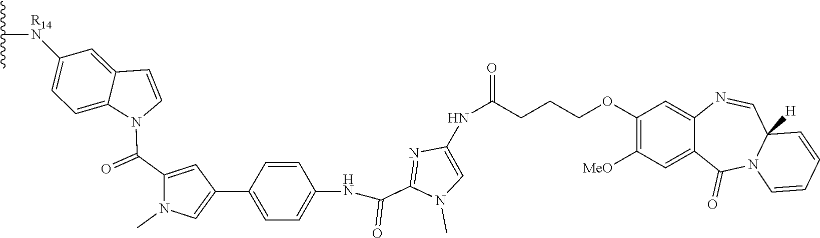

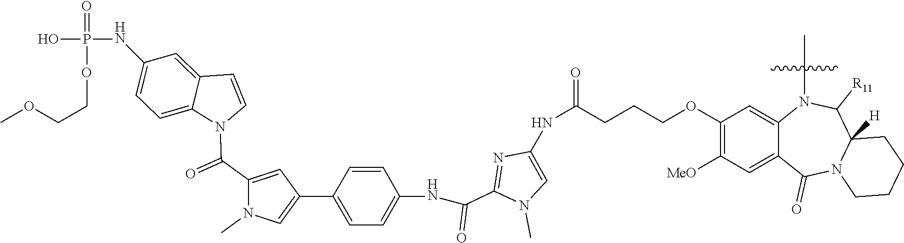

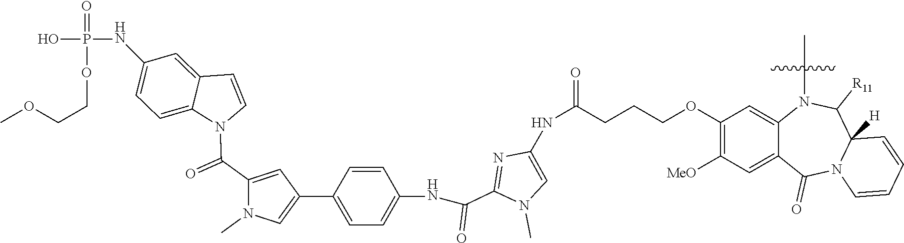

wherein R₁₁ and R₁₄ are as defined herein.

Representative examples of conjugates of Formula (I) include those listed in Table 2, a tautomer thereof, a pharmaceutically acceptable salt or solvate thereof, or a pharmaceutically acceptable salt or solvate of the tautomer. It is to be understood that d₁₃ is omitted in the conjugates listed in Table 2 and, unless specified otherwise in the corresponding Example, the value of d₁₃ is defined hereabove in the instant disclosure.

TABLE 2

| Conju- | |
|---|---|
| gate | |
| No. | Structure |
| Conju- gate No. 5 (Ex- ample 1) | 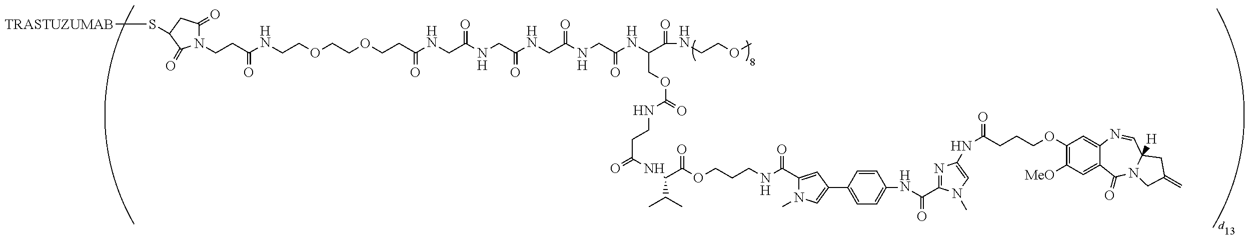 |
| Conju- gate No. 10 (Ex- ample 2) | 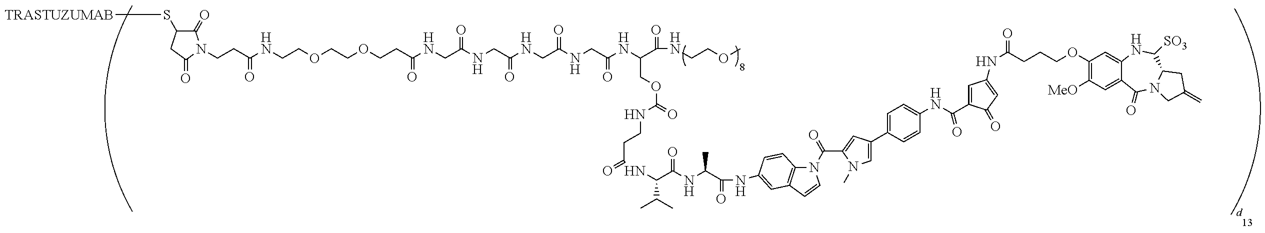 |
| Conju- gate No. 10A (Ex- ample 2A) | 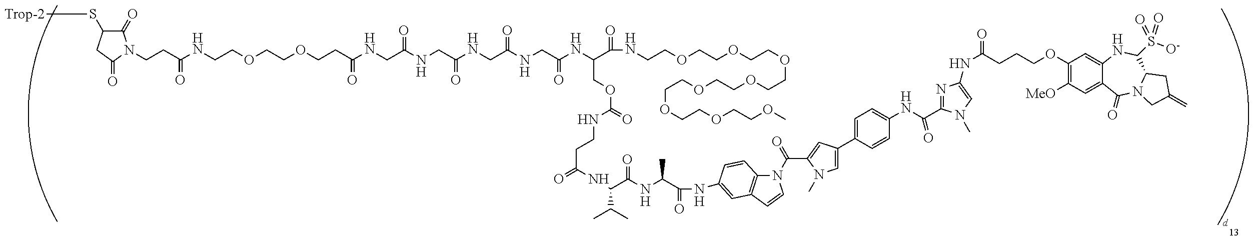 |
| Conju- gate No. 20 (Ex- ample 3) | 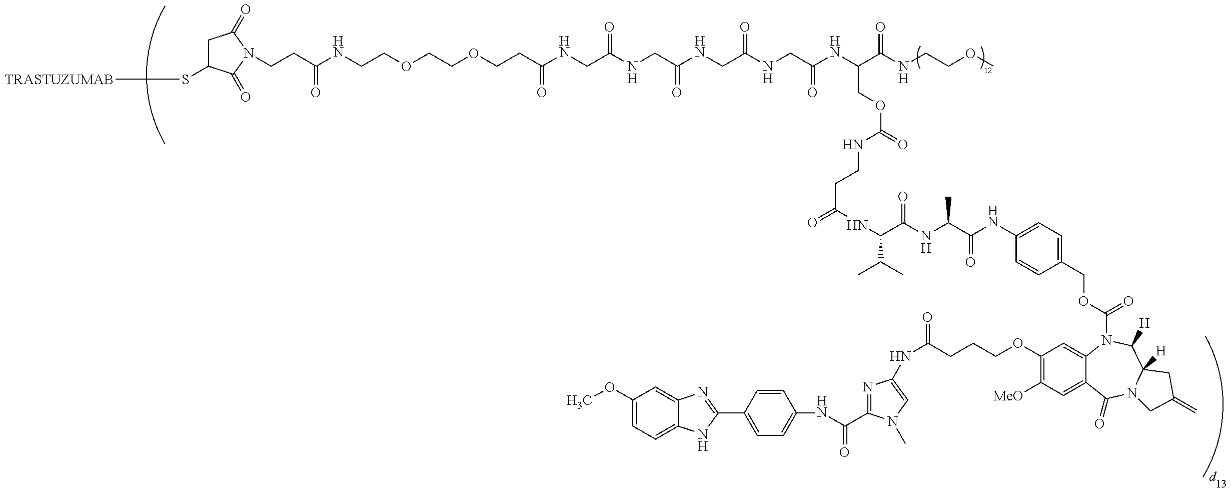 |
| Conju- gate No. 20A (Ex- ample 3A) | 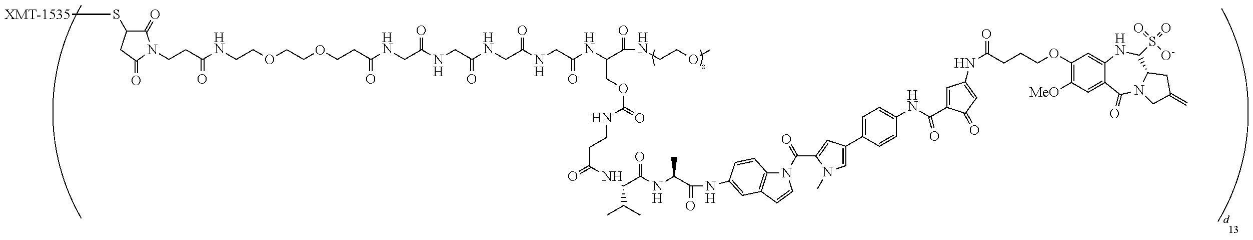 |
| Conju- gate No. 26 (Ex- ample 4) | 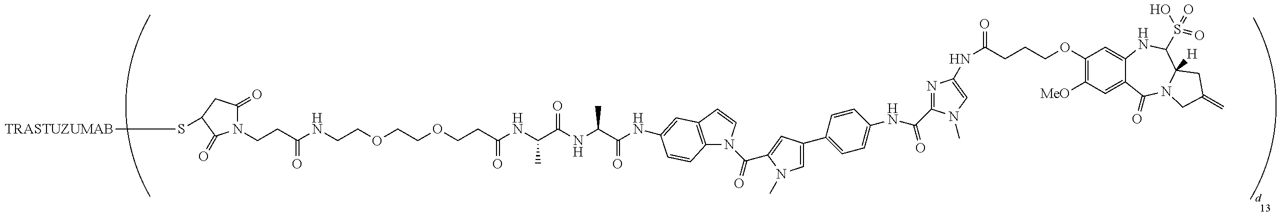 |
| Conju- gate No. 31 (Ex- ample 5) | 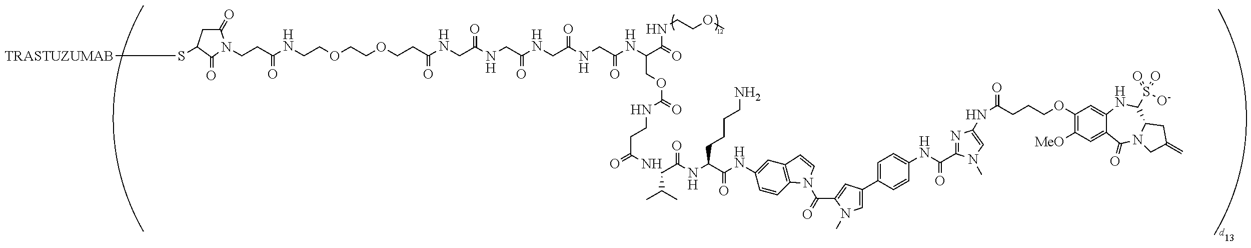 |
| Conju- gate No. 36 (Ex- ample 6) | 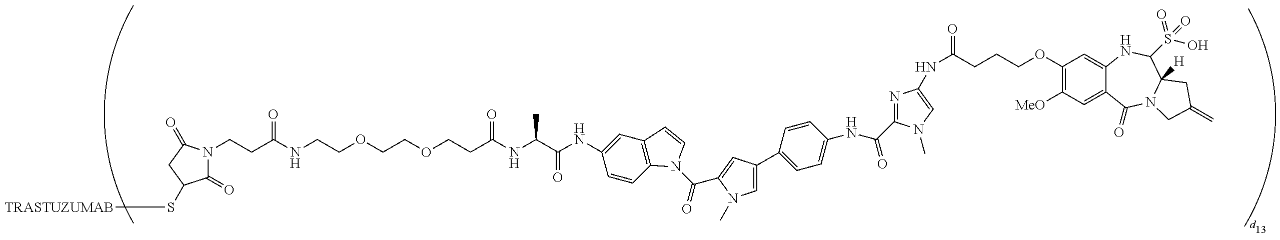 |
| Conju- gate No. 38 (Ex- ample 7) | 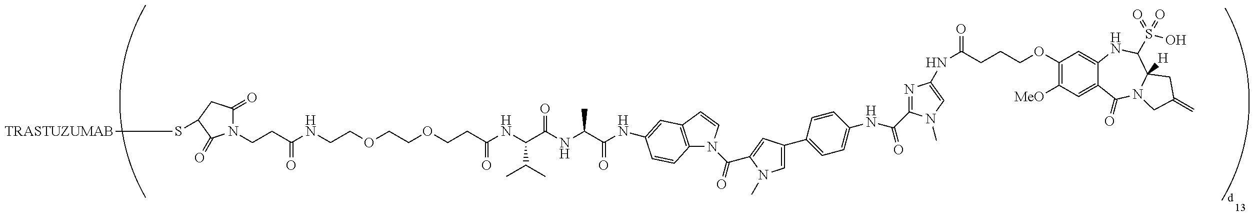 |
| Conju- gate No. 46 (Ex- ample 8) | 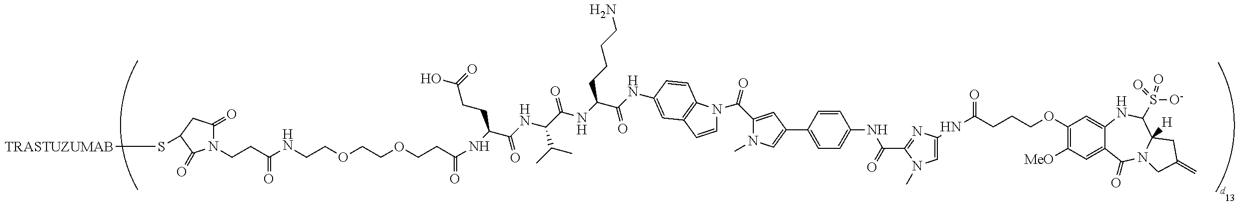 |
| Conju- gate No. 57 (Ex- ample 9) | 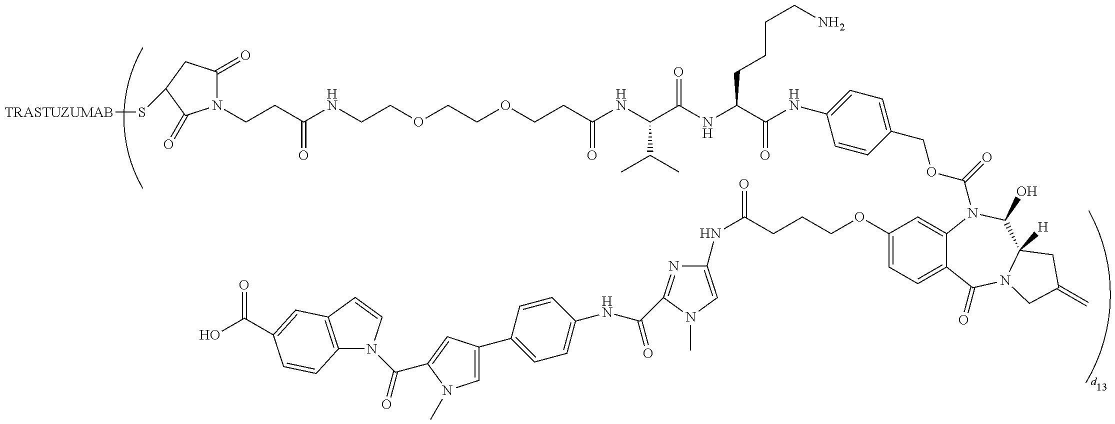 |
| Conju- gate No. 60 (Ex- ample 10) | 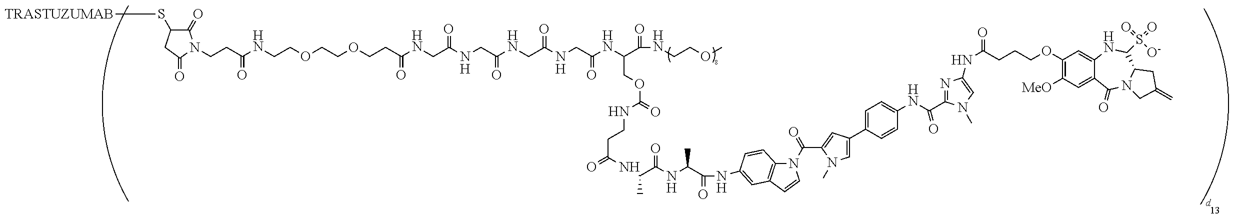 |
| Conju- gate No. 61 (Ex- ample 11) | 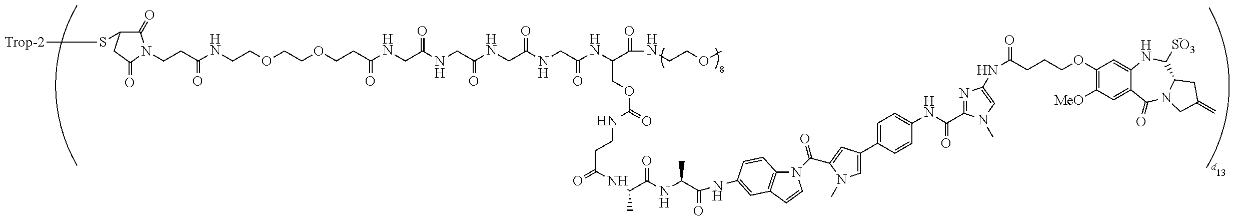 |
| Conju- gate No. 62 (Ex- ample 12) | 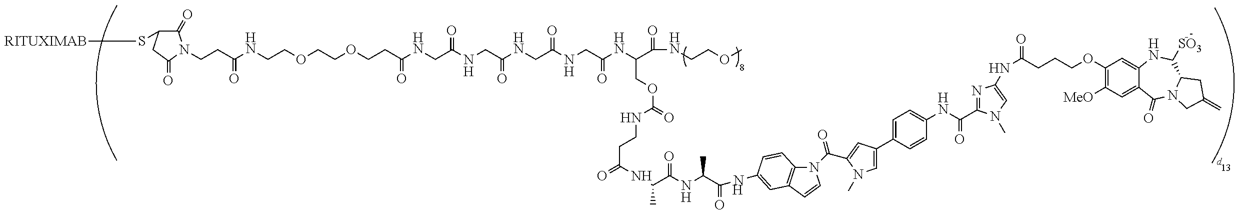 |
| Conju- gate No. 67 (Ex- ample 15) | 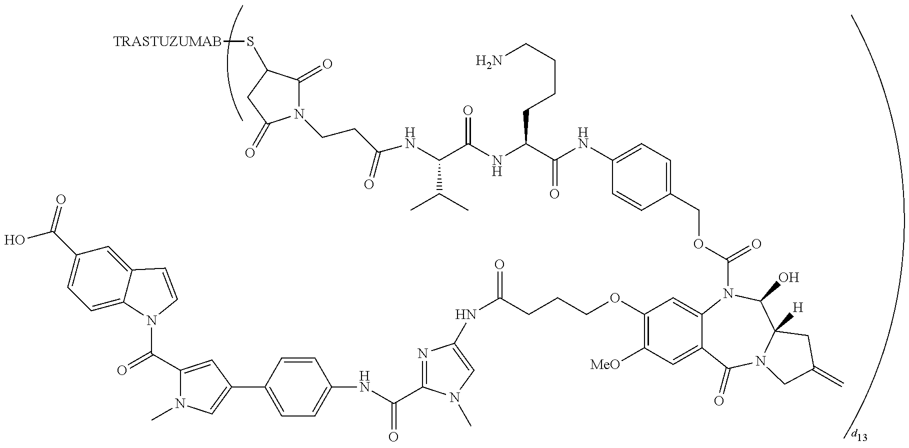 |
| Conju- gate No. 71 (Ex- ample 16) | 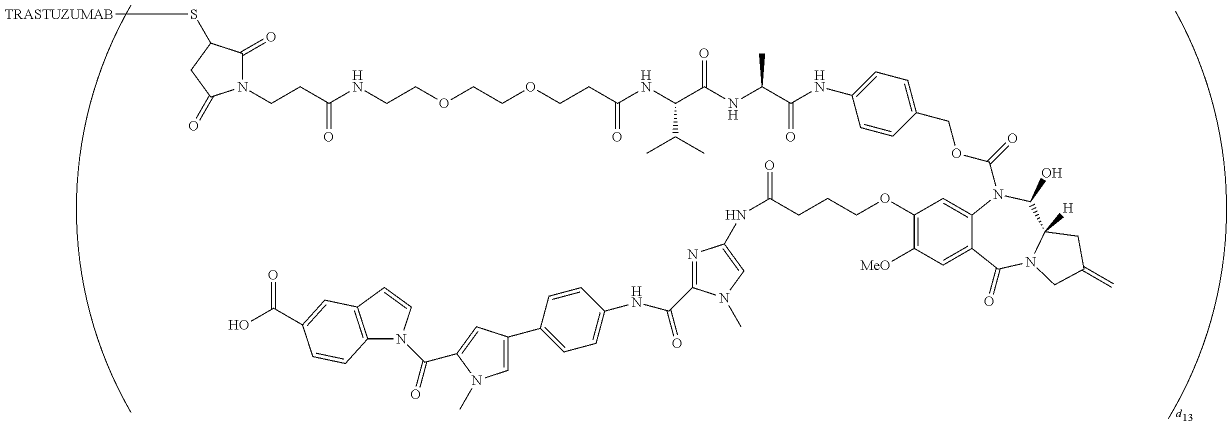 |
| Conju- gate No. 73 (Ex- ample 17) | 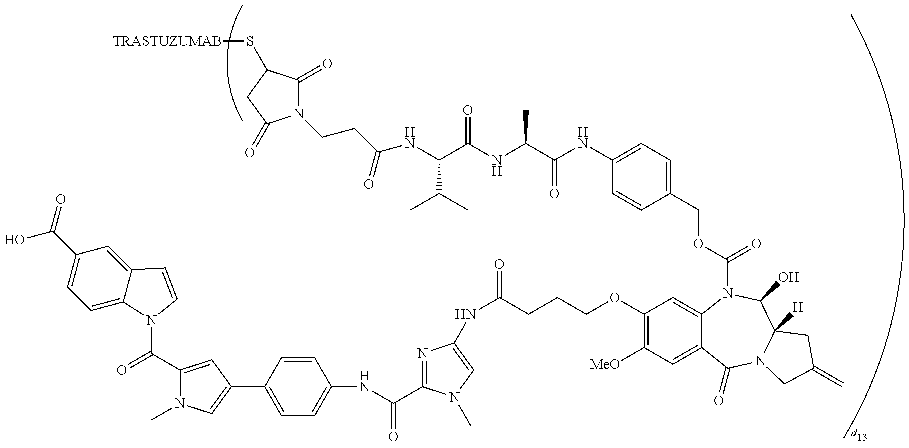 |
| Conju- gate No. 73A (Ex- ample 17A) | 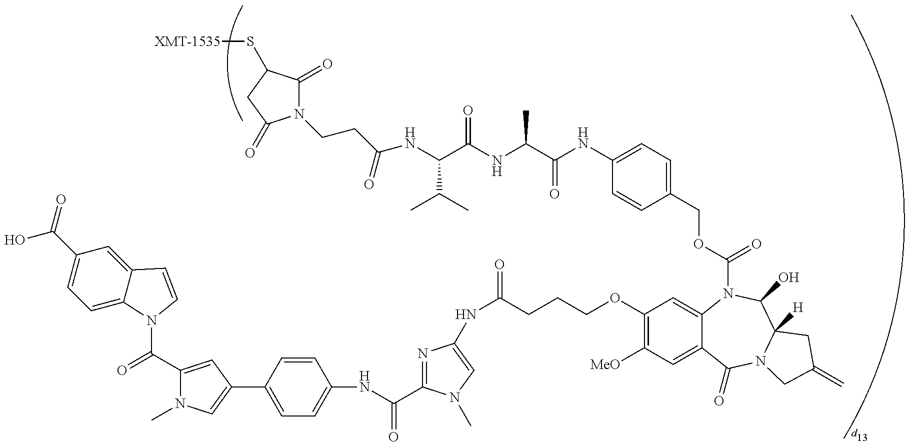 |
| Conju- gate No. 79 (Ex- ample 18) | 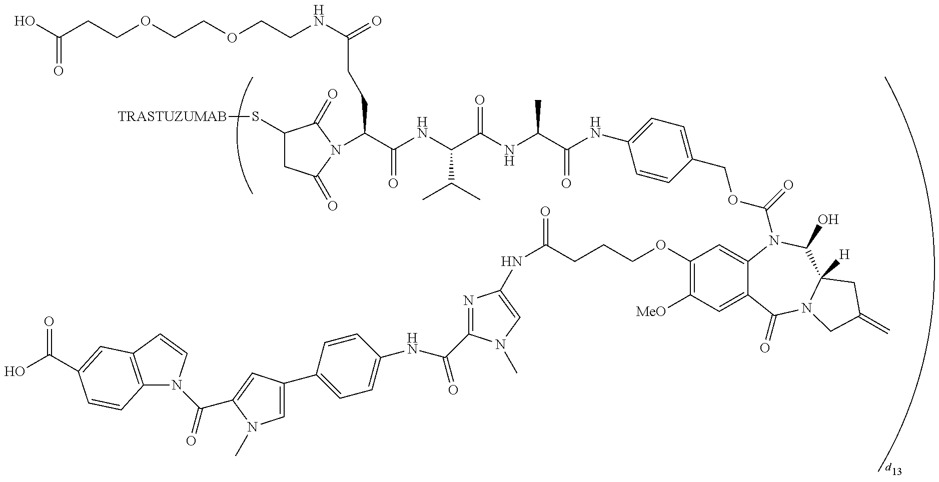 |
| Conju- gate No. 86 (Ex- ample 19) | 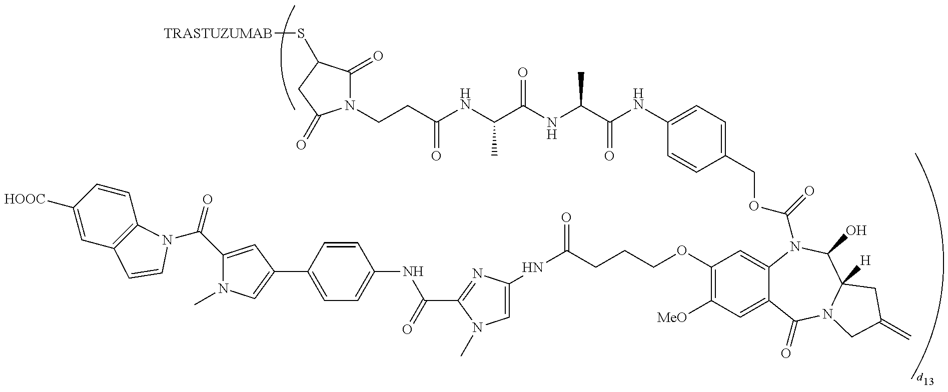 |
| Conju- gate No. 94 (Ex- ample 20) | 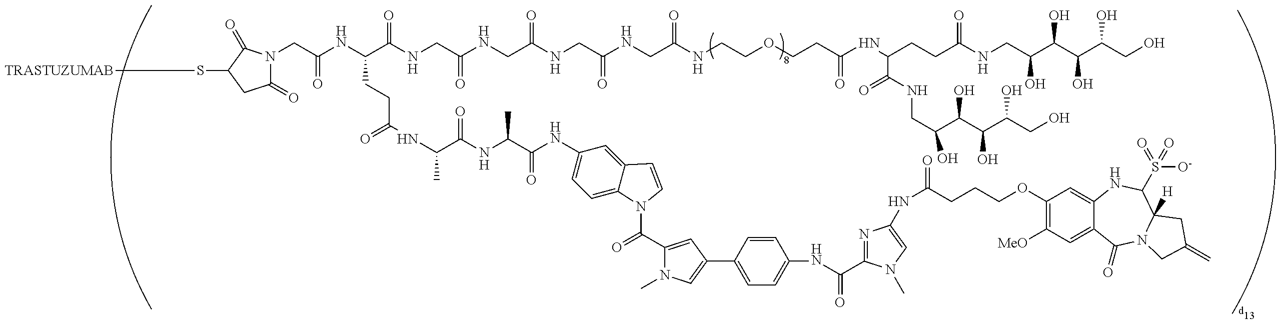 |
| Conju- gate No. 94A (Ex- ample 20A) | 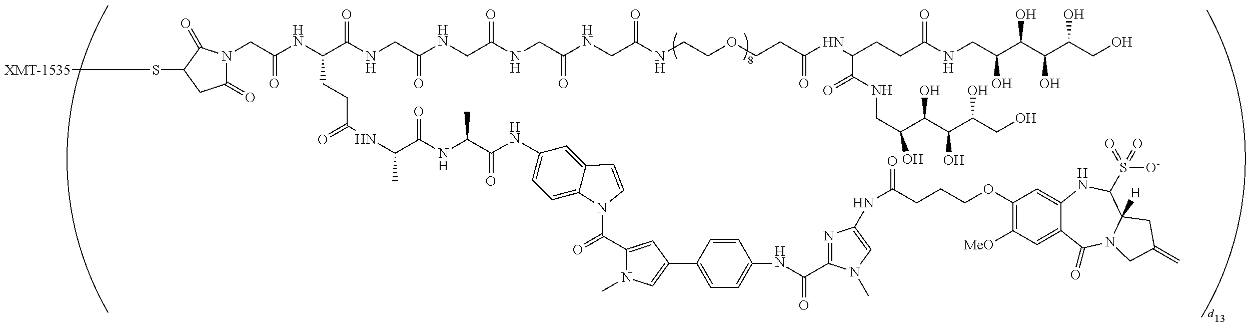 |
| Conju- gate No. 94B (Ex- ample 20B) | 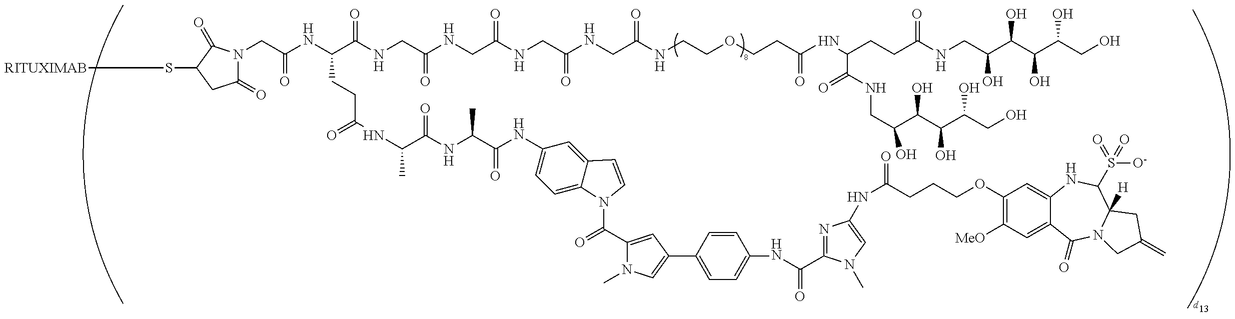 |
| Conju- gate No. 105 (Ex- ample 21) | 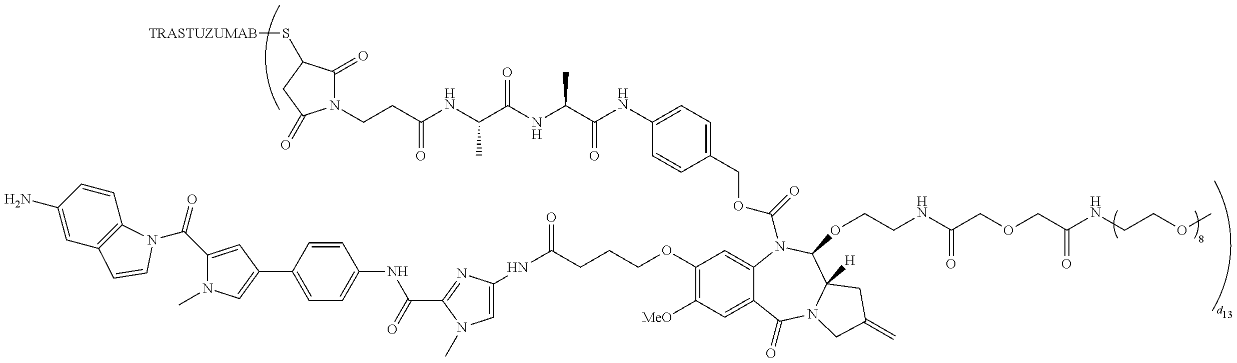 |
| Conju- gate No. 112 (Ex- ample 22) | 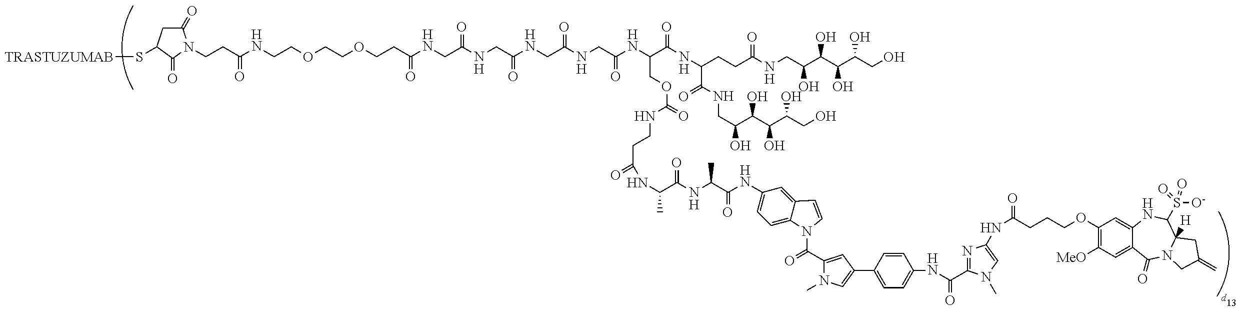 |
| Conju- gate No. 115 (Ex- ample 23) | 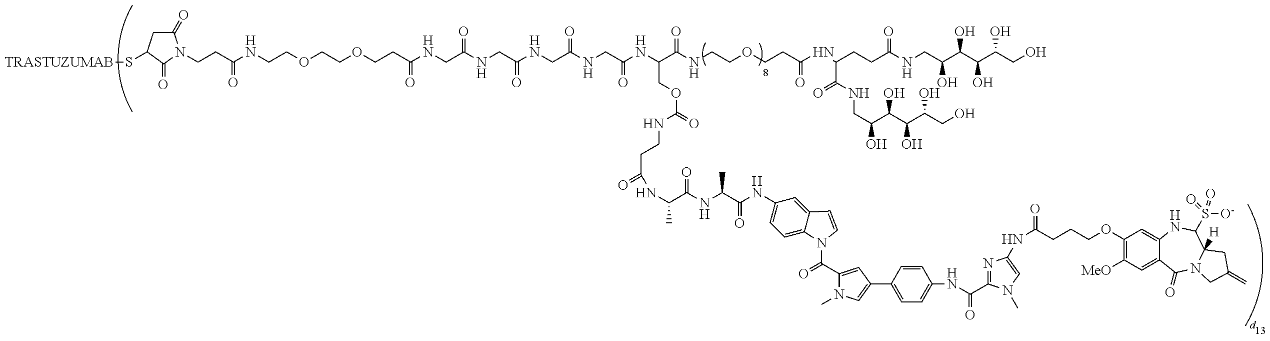 |
| Conju- gate No. 119 (Ex- ample 24) | 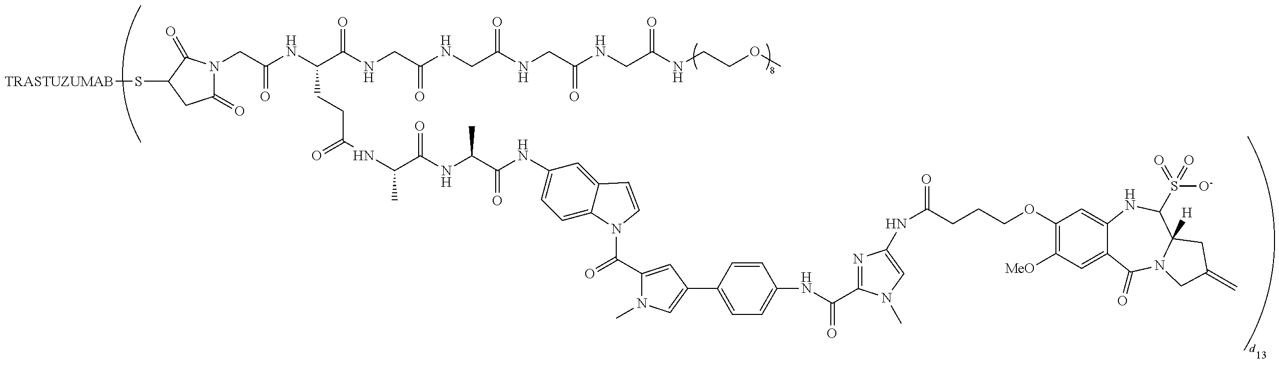 |
| Conju- gate No. 122 (Ex- ample 25) | 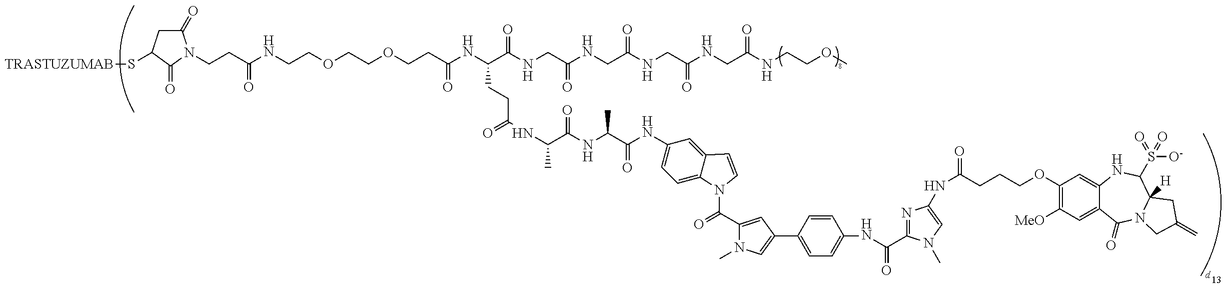 |
| Conju- gate No. 130 (Ex- ample 26) | 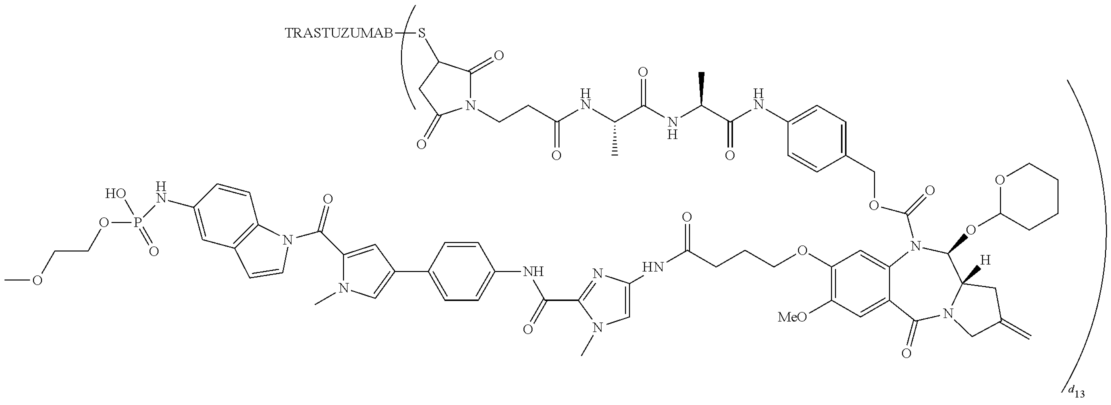 |
| Conju- gate No. 135 (Ex- ample 27) | 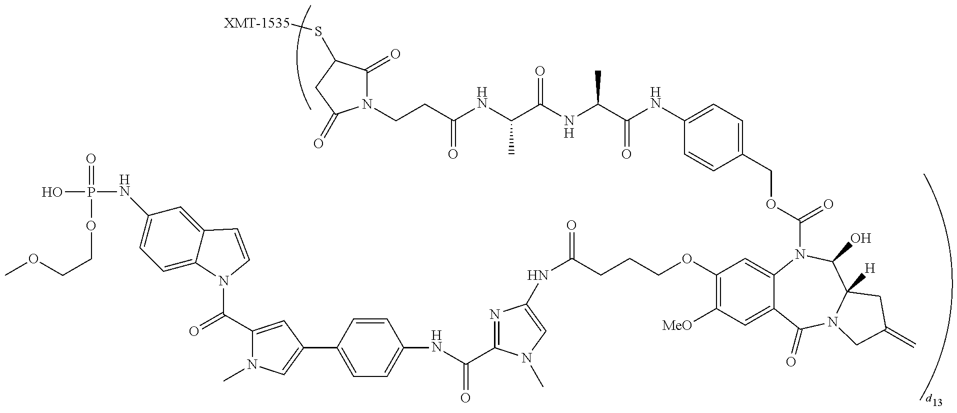 |
| Conju- gate No. 135A (Ex- ample 27A) | 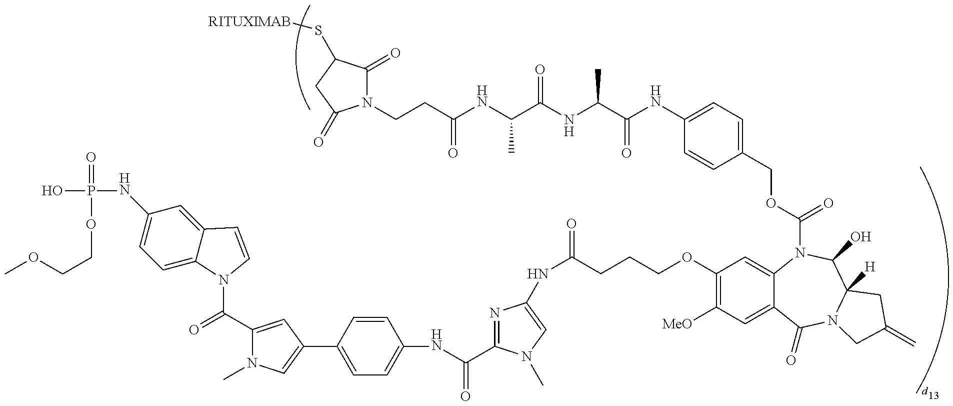 |
| Conju- gate No. 136 (Ex- ample 28) | 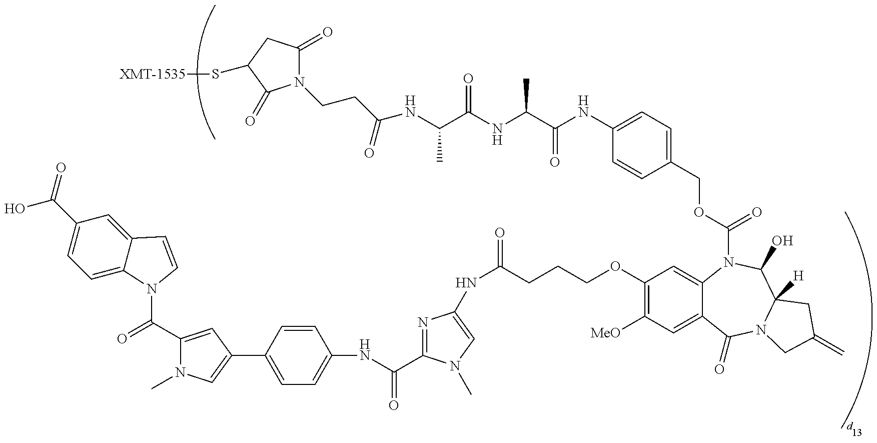 |
| Conju- gate No. 136A (Ex- ample 28A) | 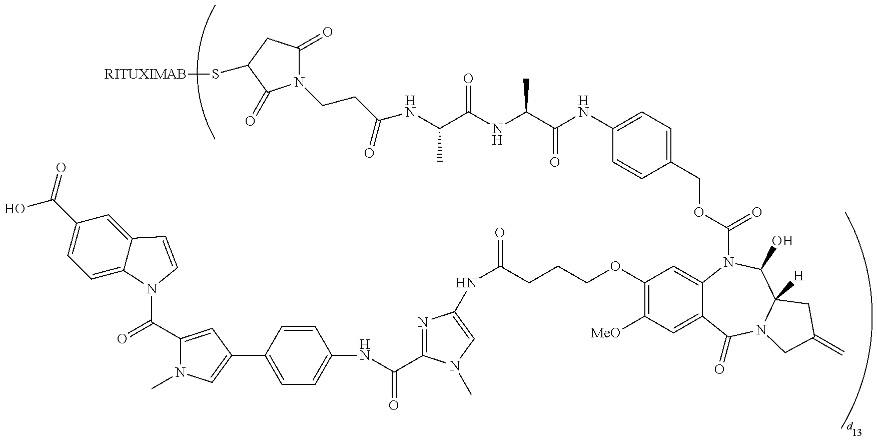 |
| | 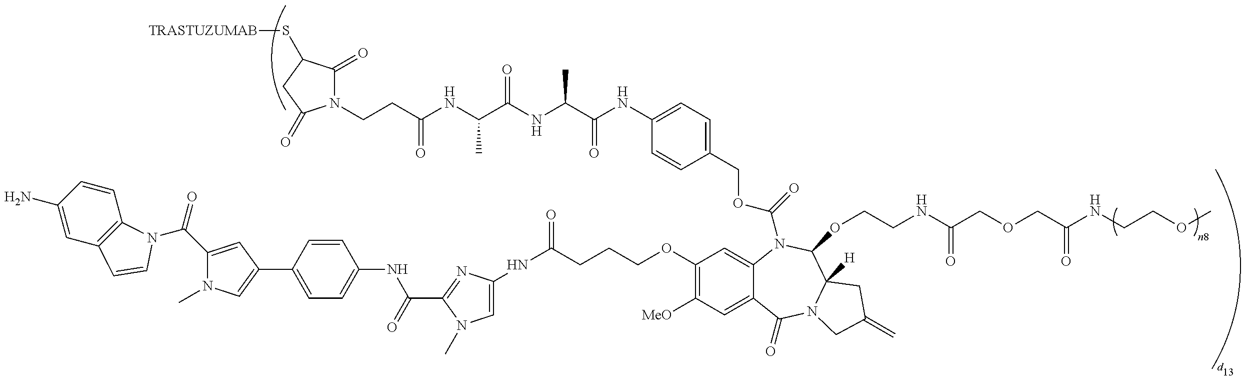 |
| | 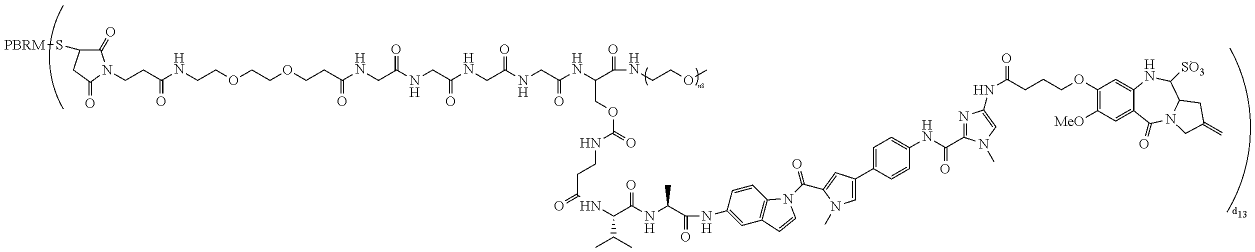 |
| | 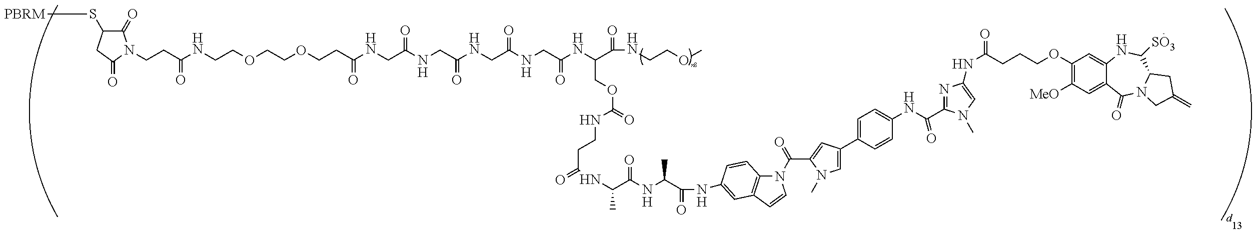 |
| | 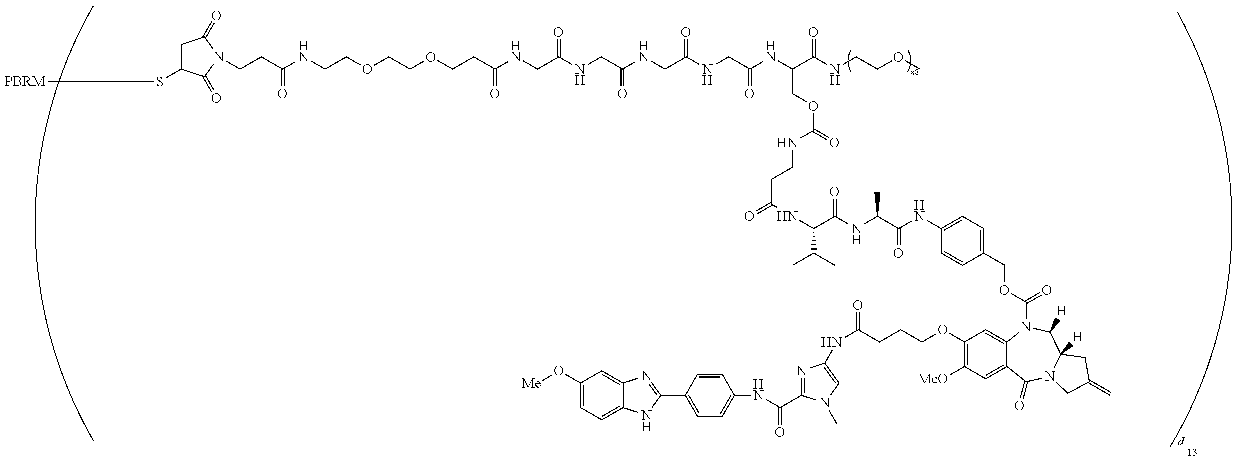 |
| | 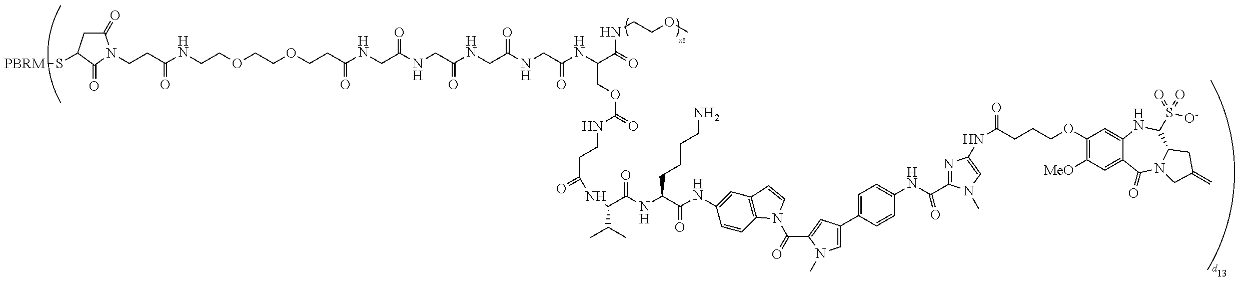 |
| | 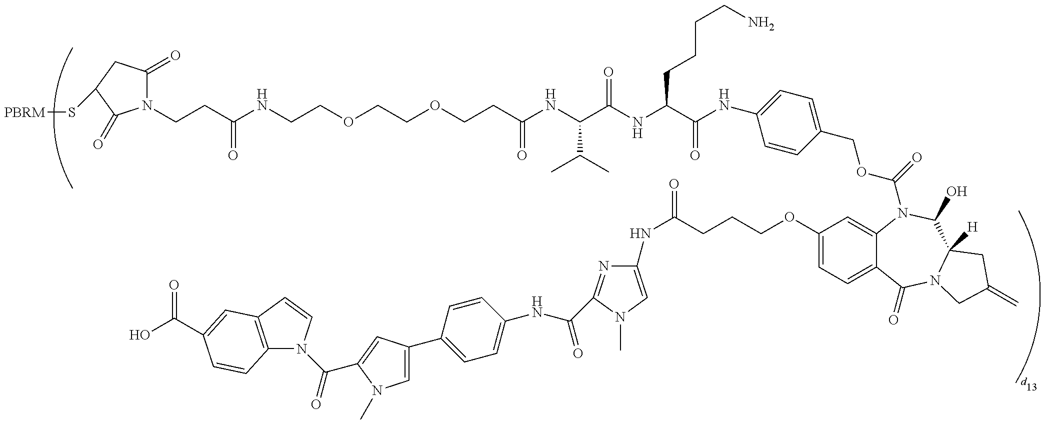 |
| | 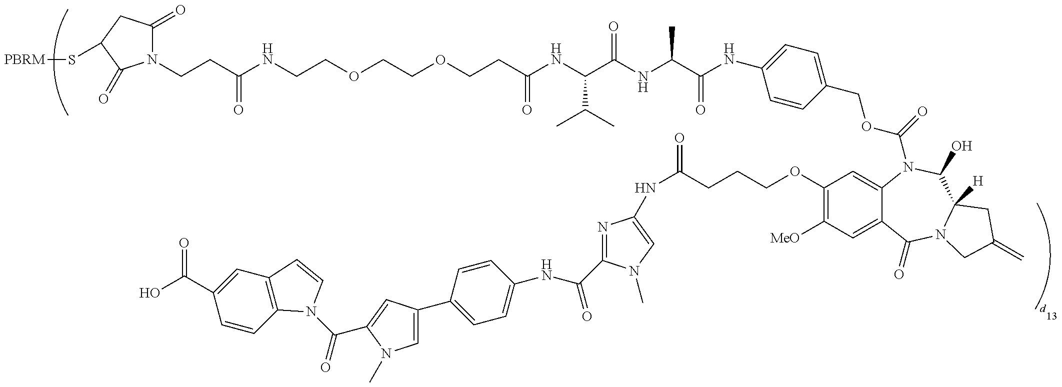 |
| | 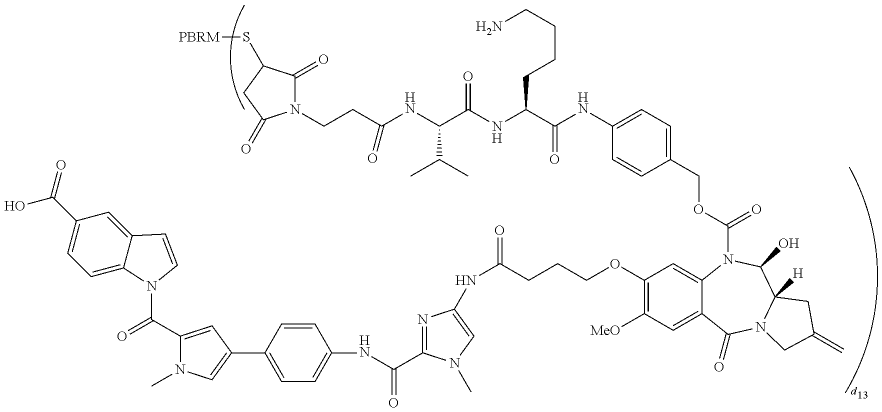 |
| | 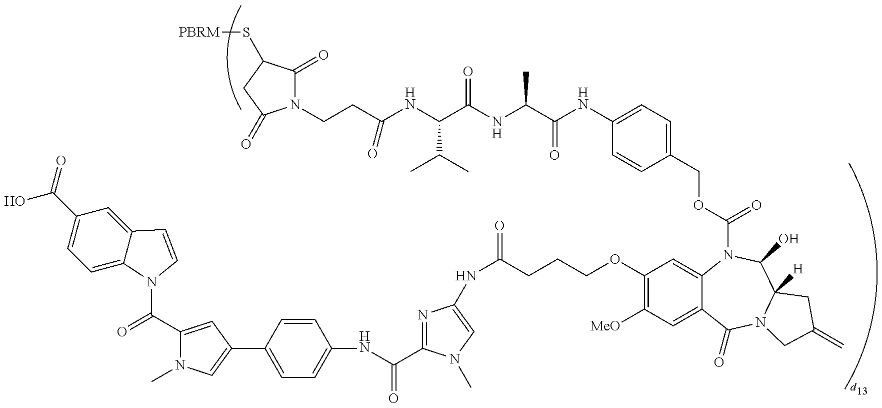 |
| | 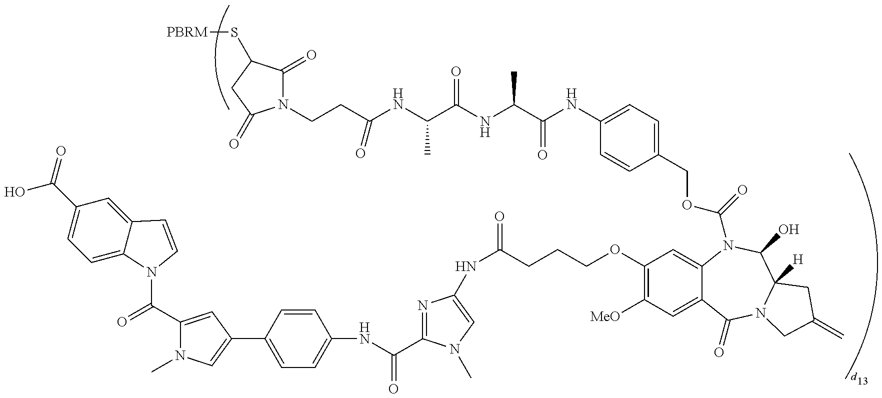 |
| | 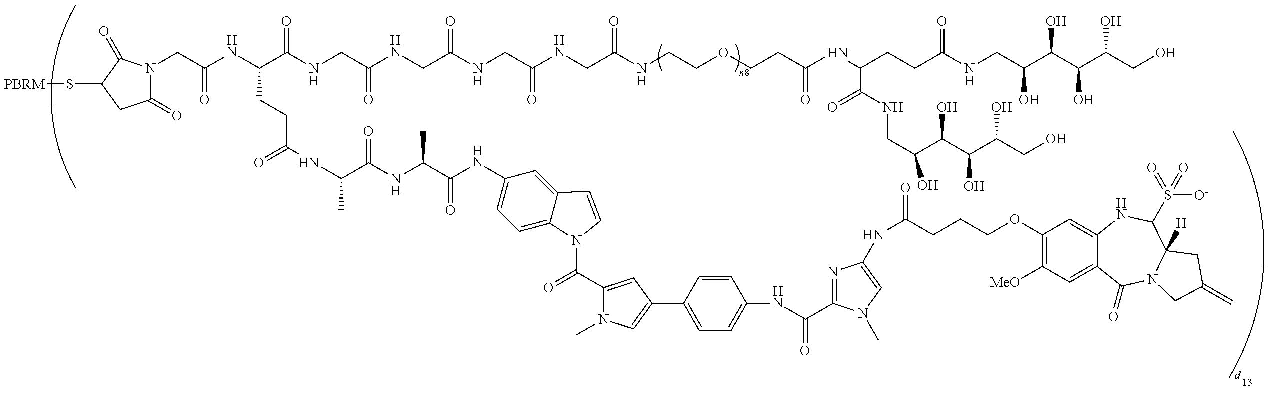 |
| | 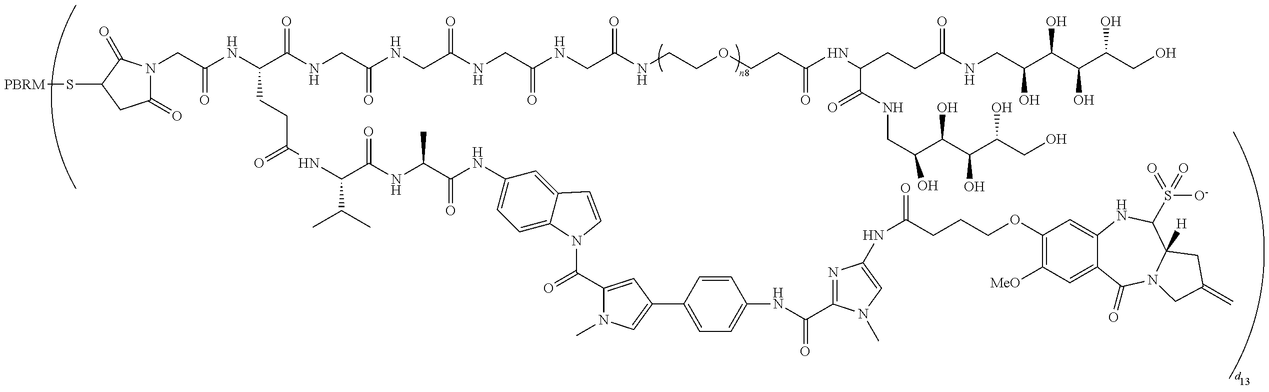 |
| | 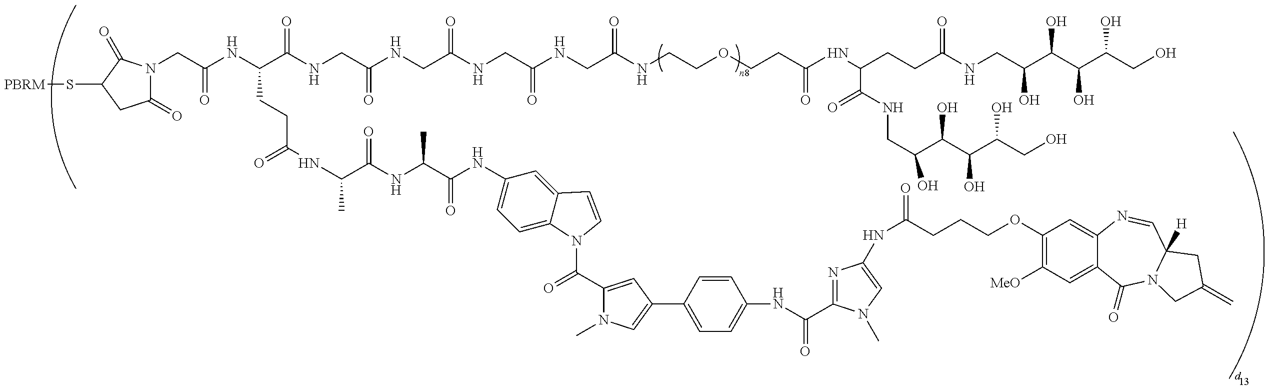 |
| | 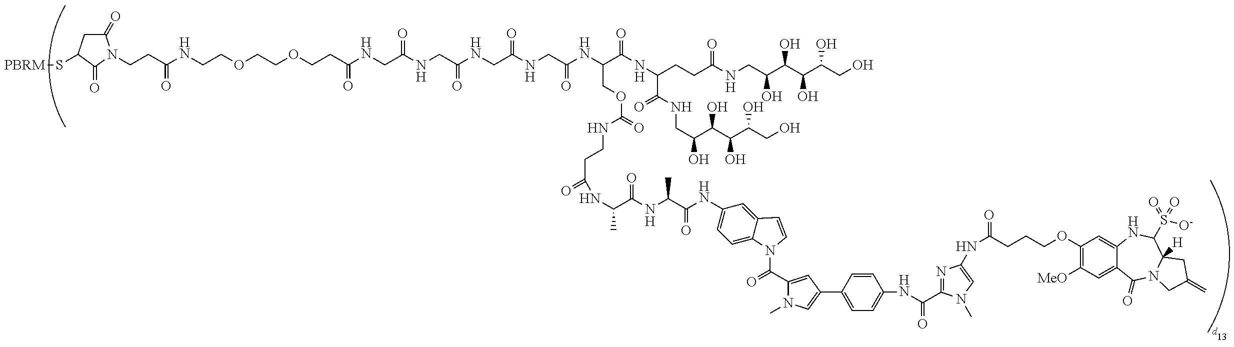 |
| | 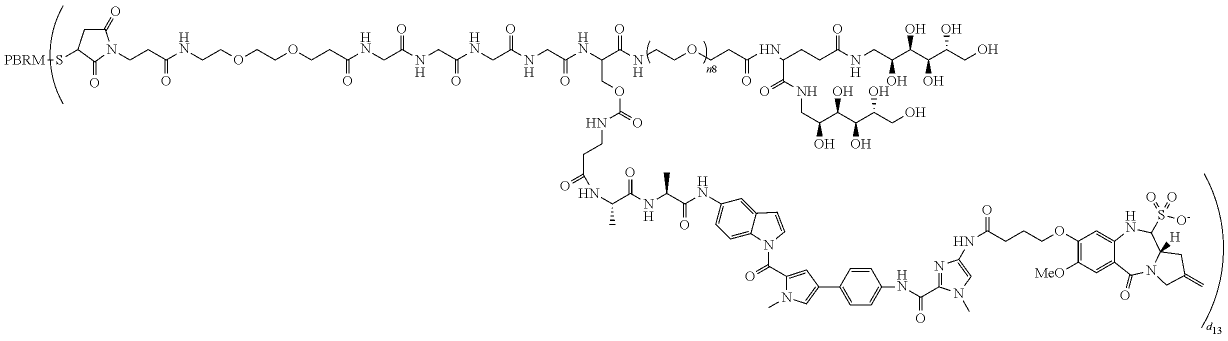 |
| | 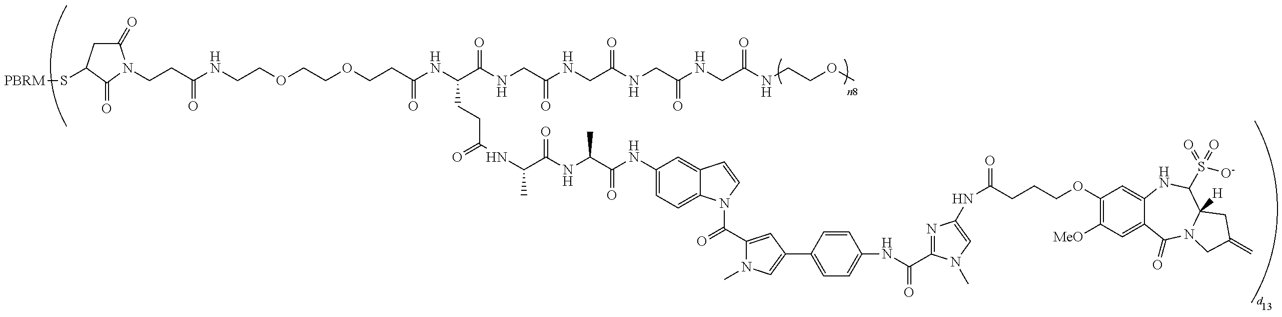 |
| | 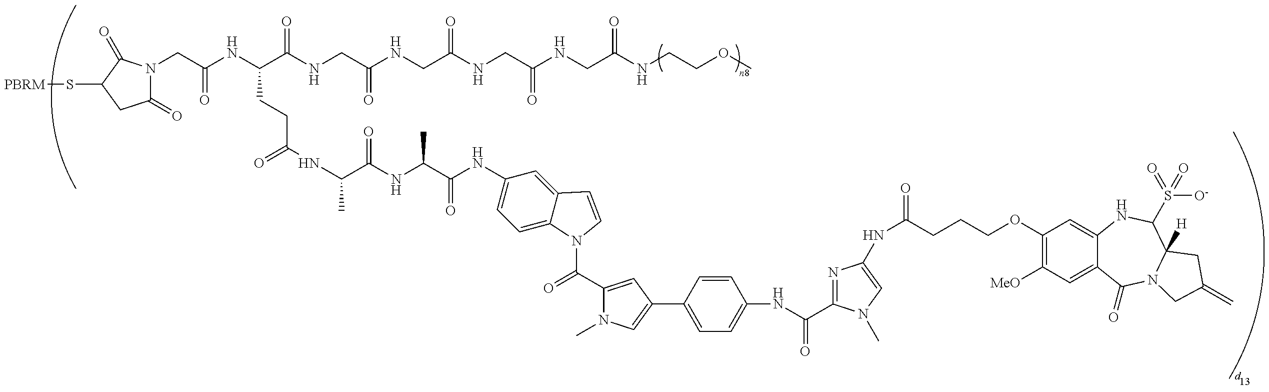 |
| | 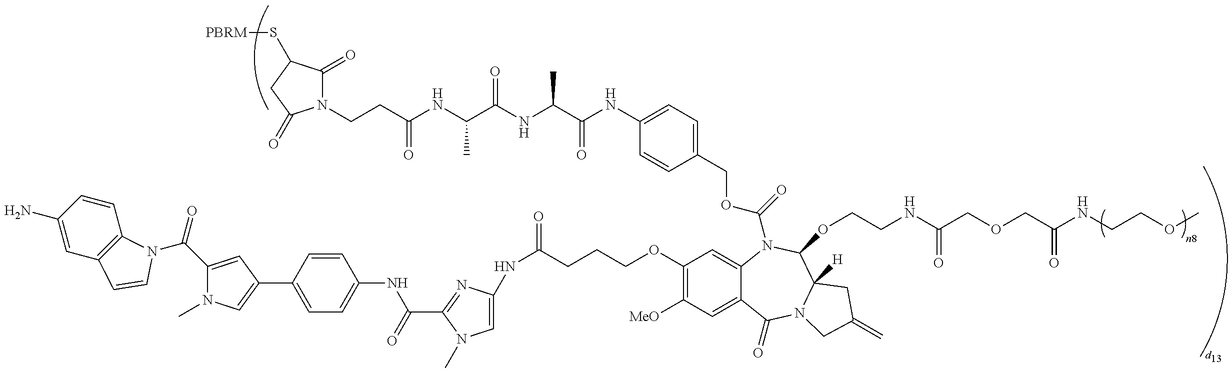 |
| | 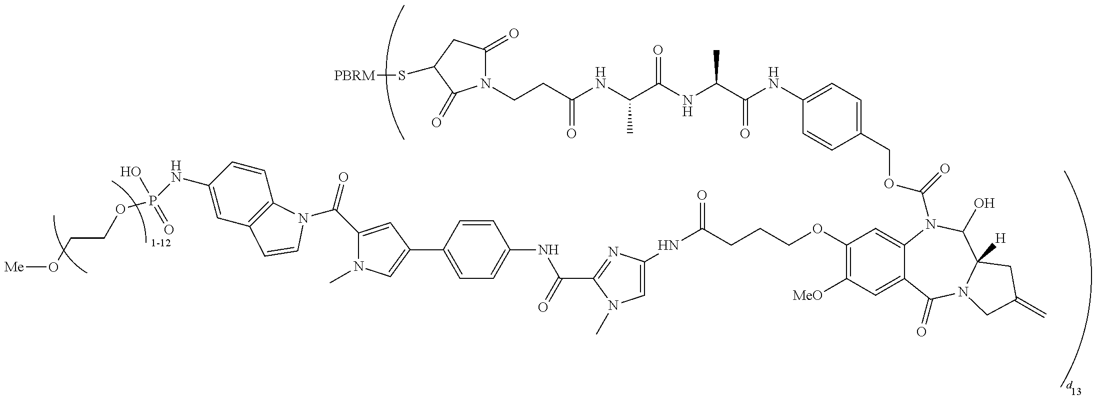 |
| | 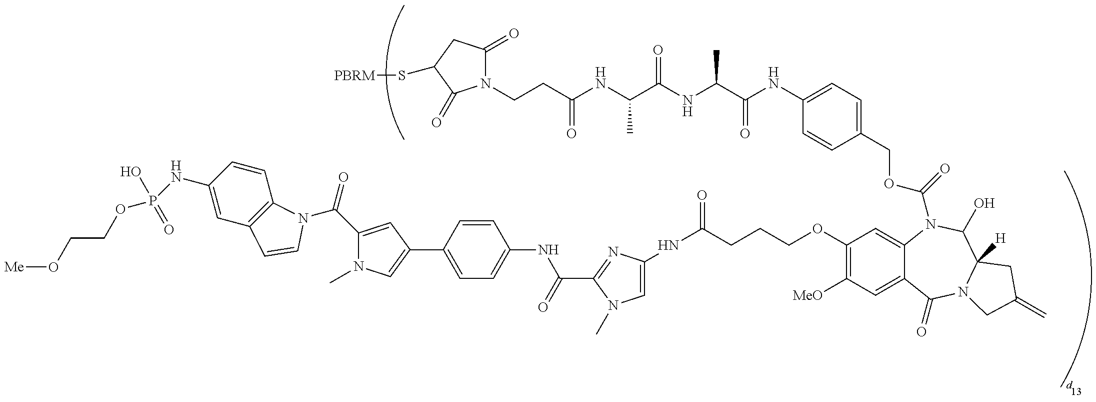 |
| | 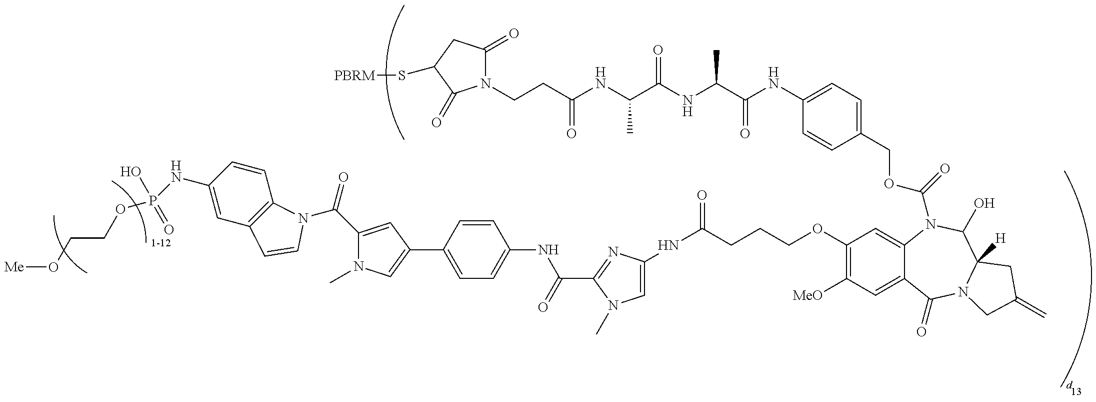 |
| | 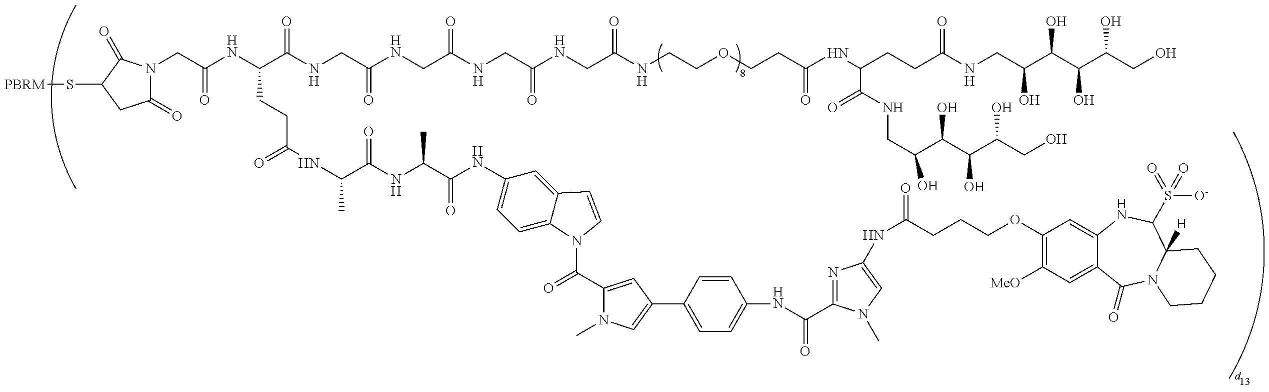 |
| | 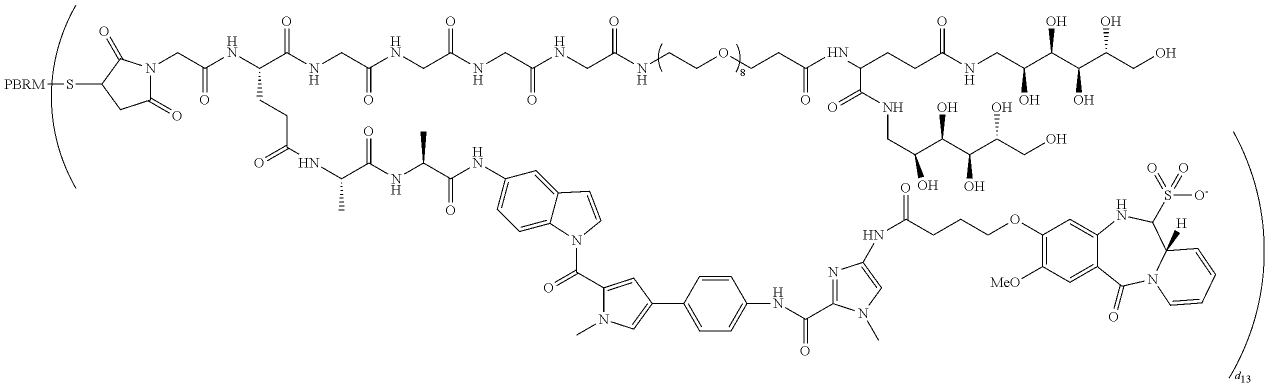 |
| | 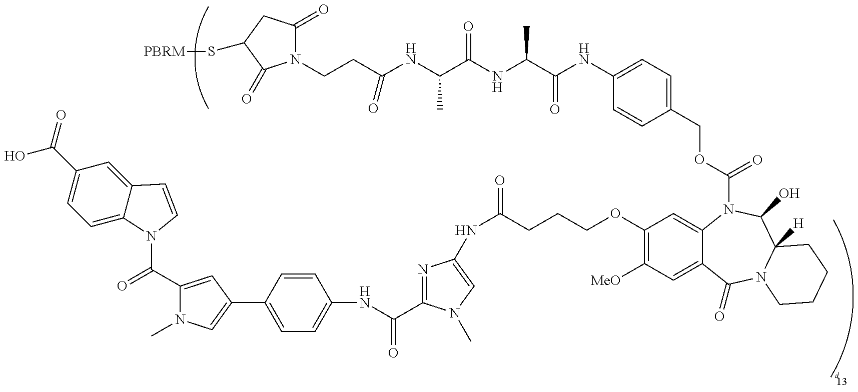 |
| | 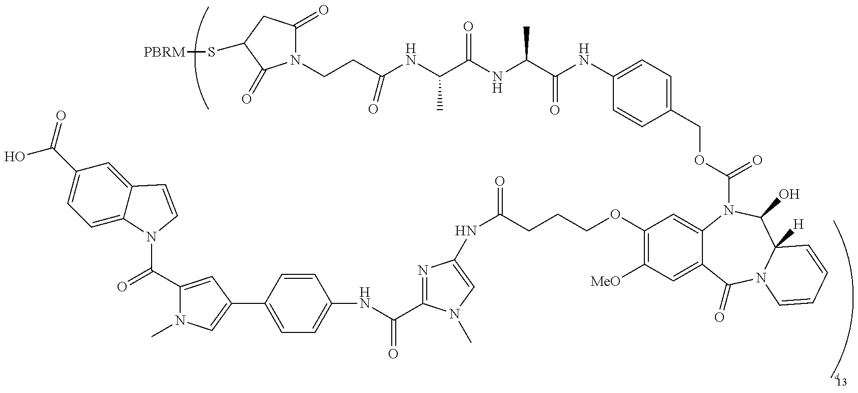 |
| | 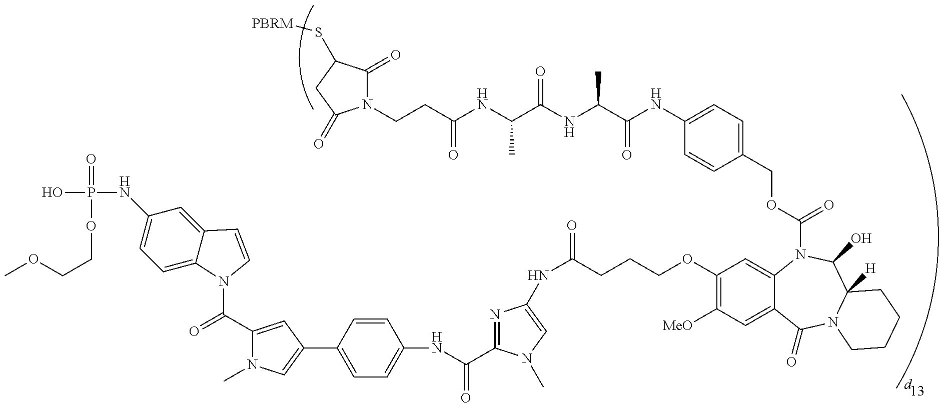 |
| | 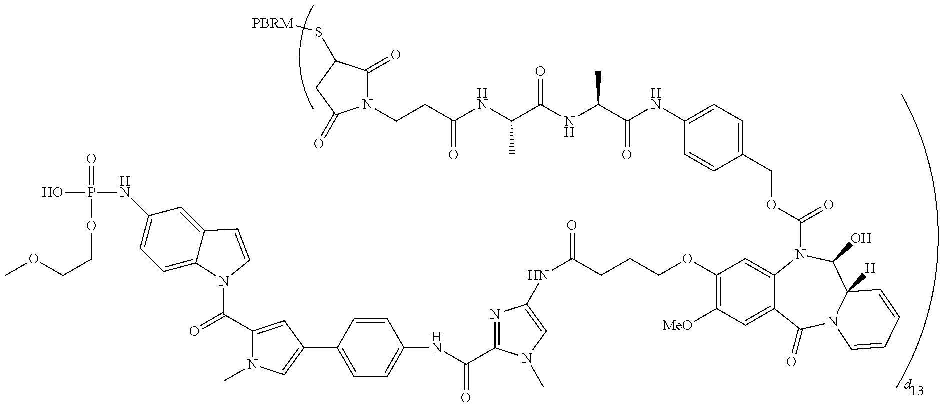 |
| | 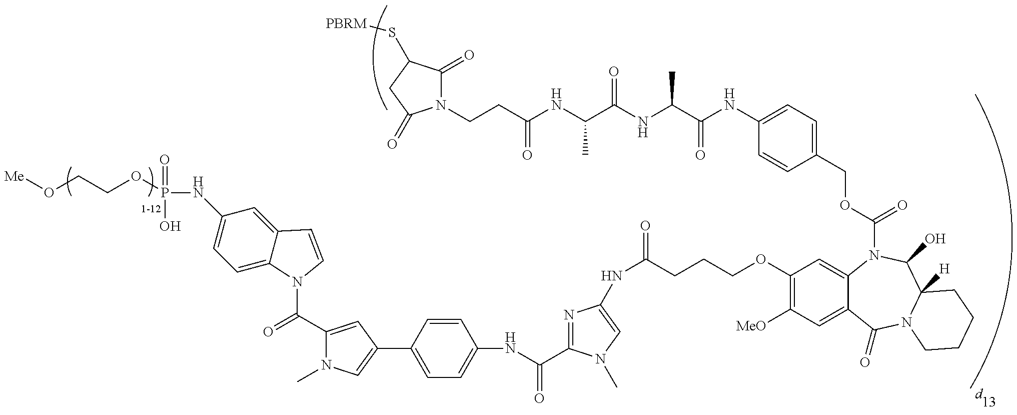 |
| | 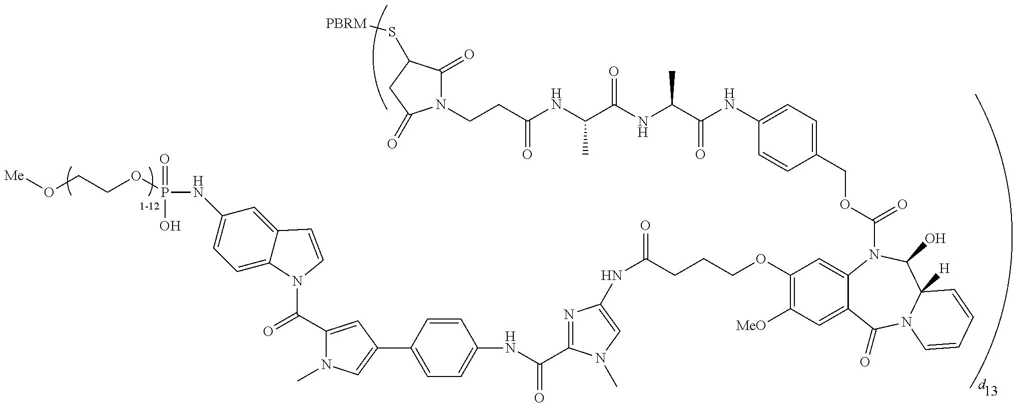 |
| | 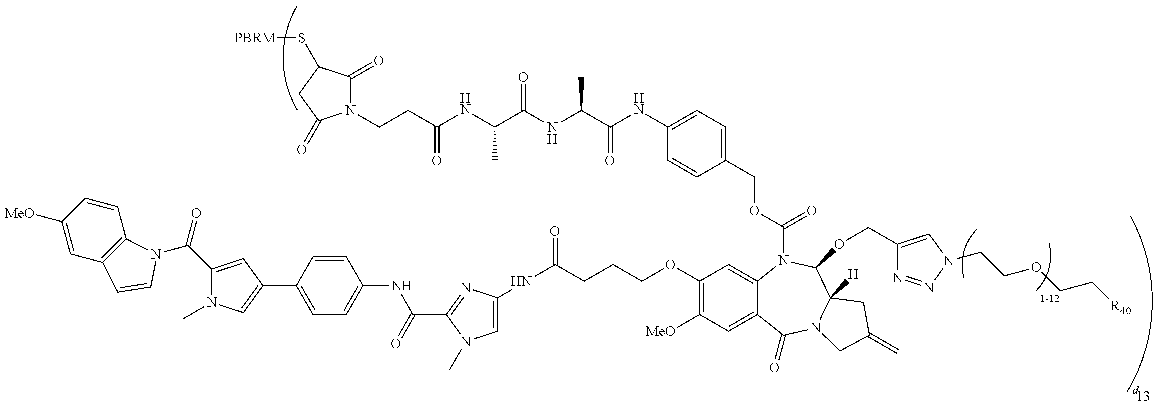 |
| | 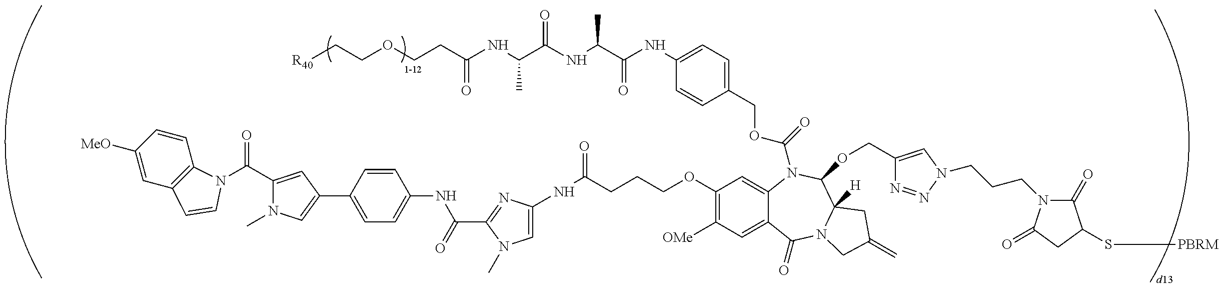 |
| | 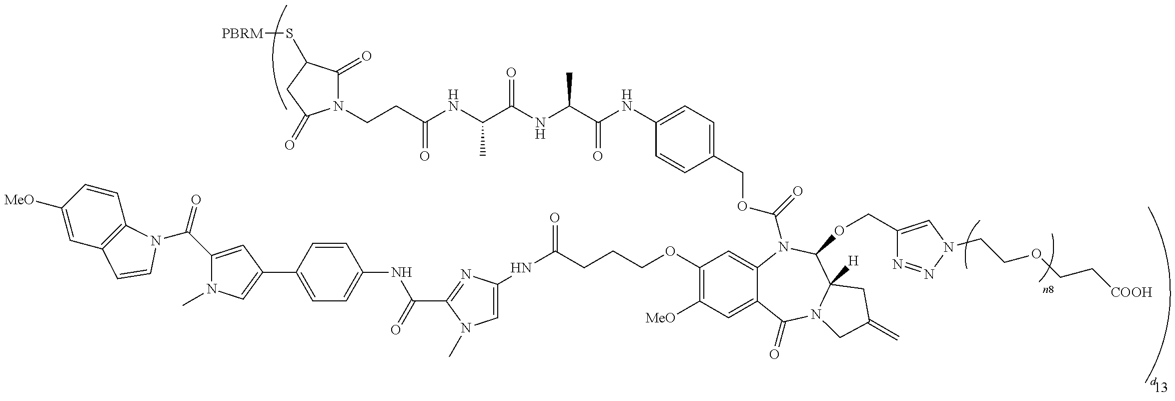 |
| | 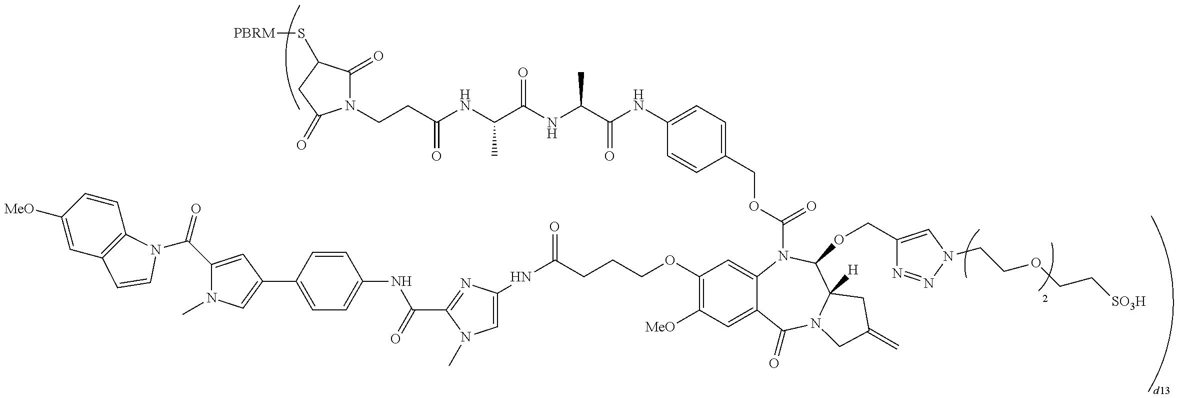 |
| | 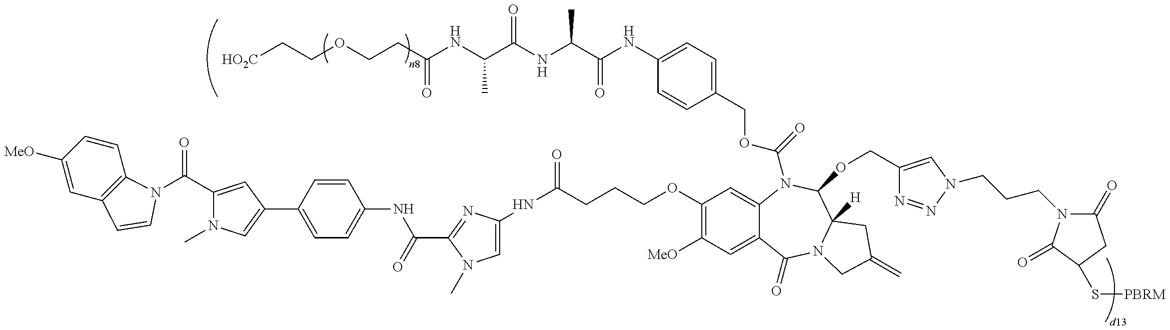 |
| | 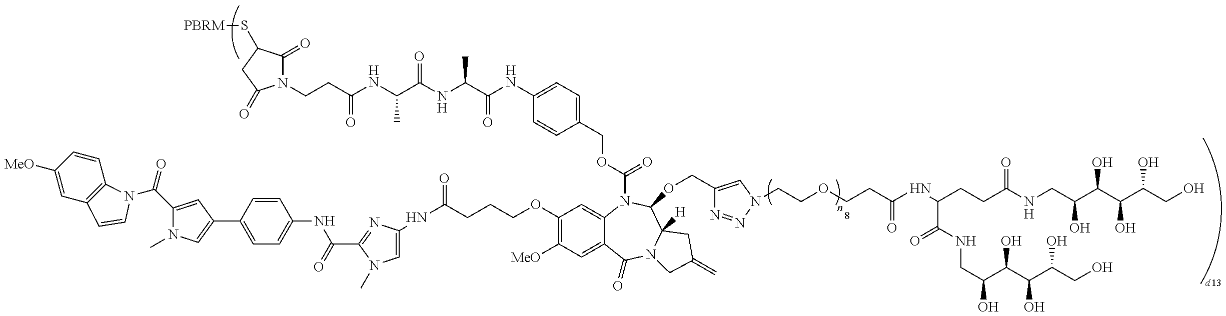 |
| | 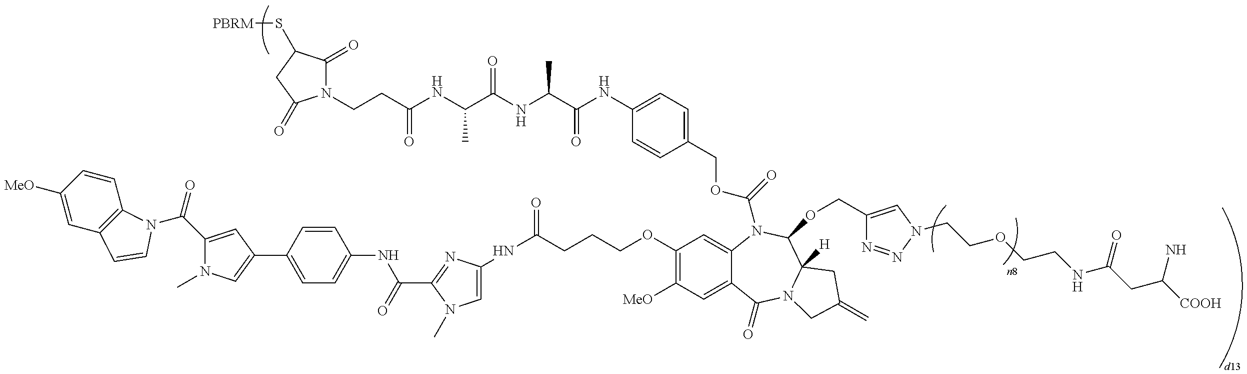 |
| | 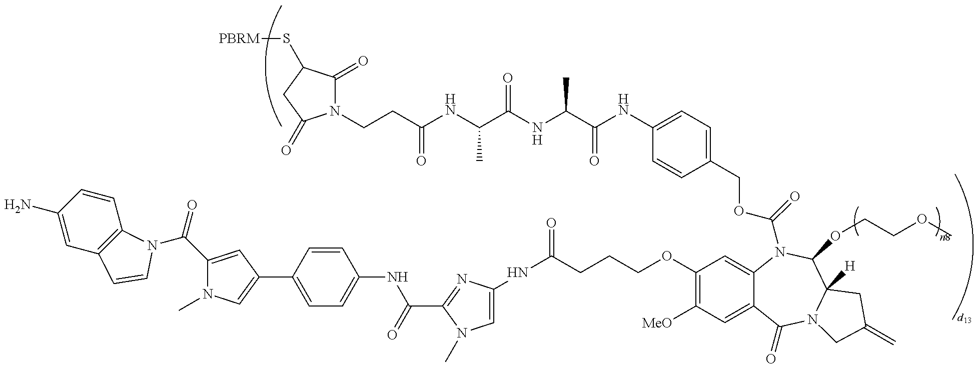 |
| | 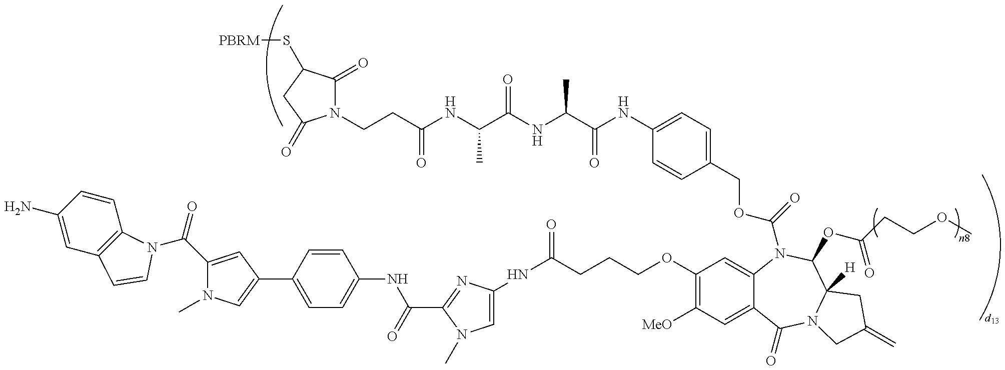 |
| | 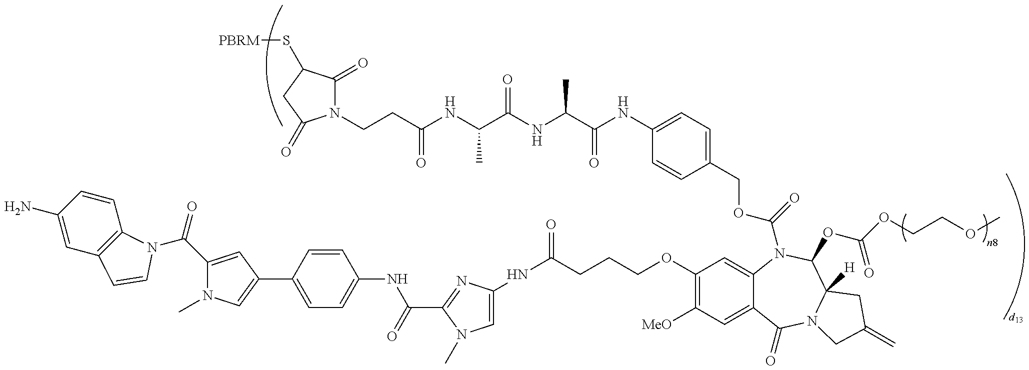 |
| | 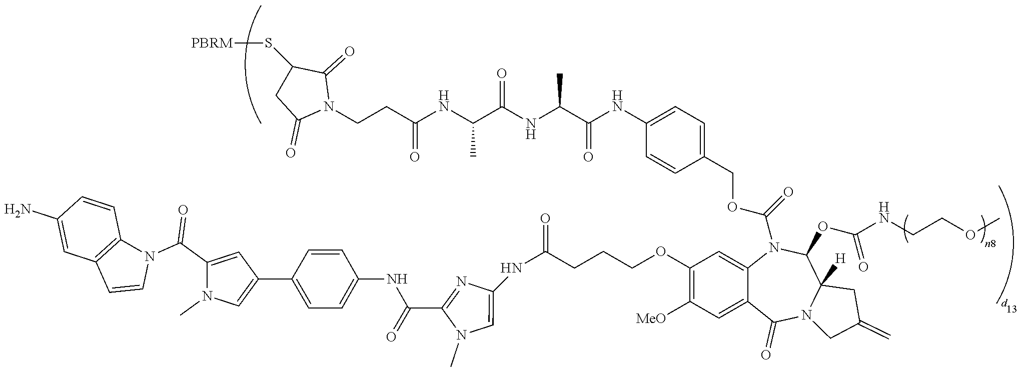 |
| | 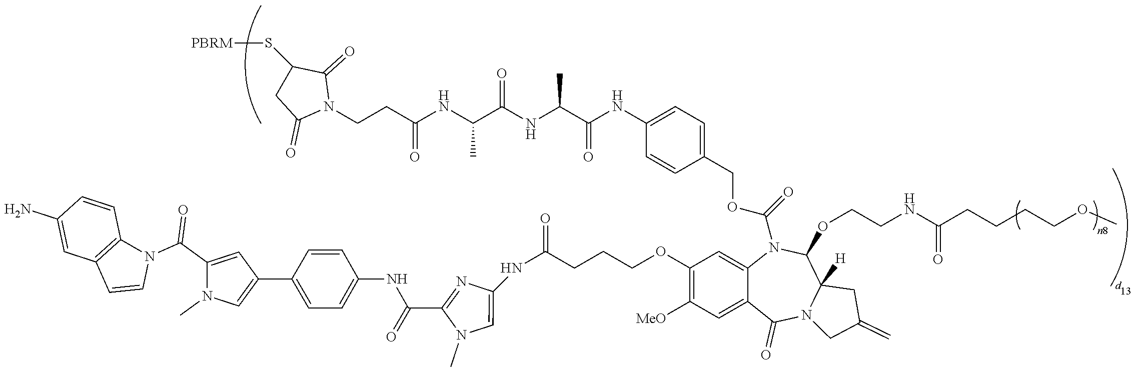 |

wherein

R₄₀ is —SO₃H, —COOH, —C(O)NH(CH₂)₂SO₃H or —C(O)NH(CH₂)₂COOH;

ng is 6, 8, or 12, and preferably, du is 3 to 5.

In some embodiments, the PBD conjugates is a conjugate of any one of Formulae (XIV-a) to (XIVx):

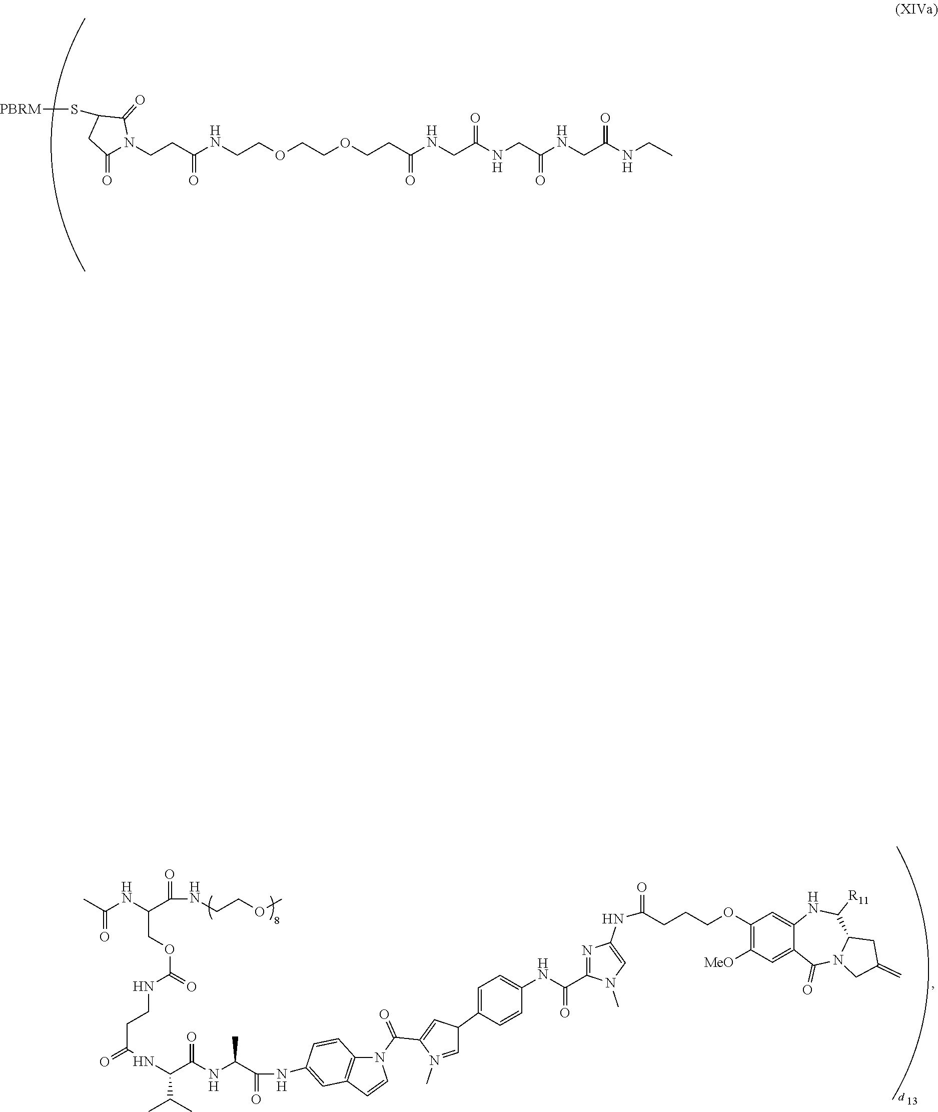

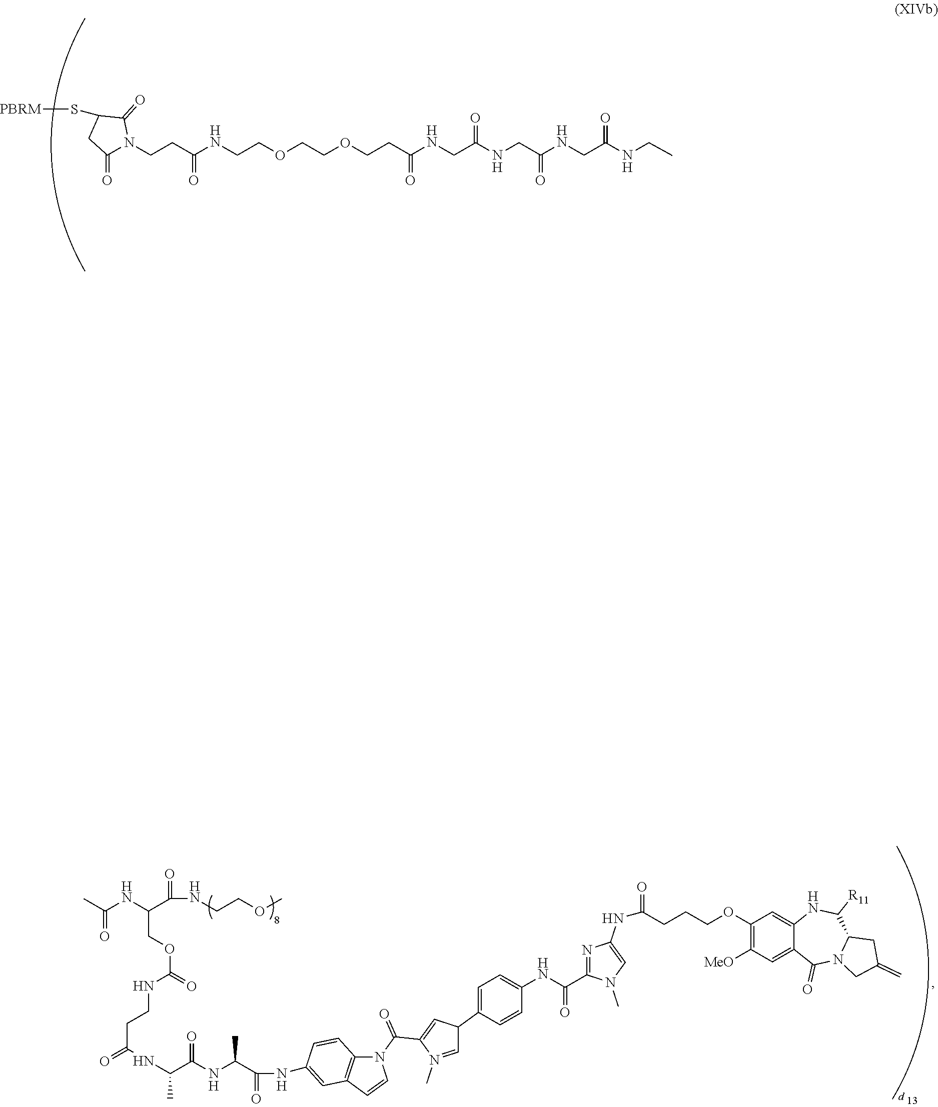

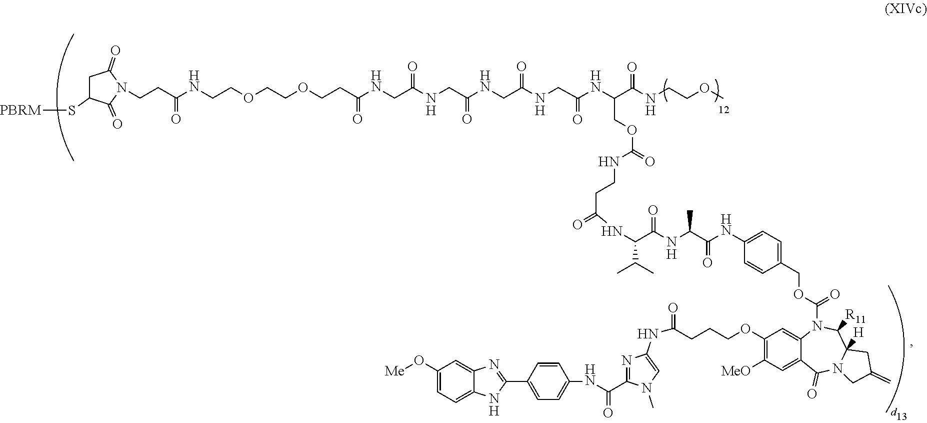

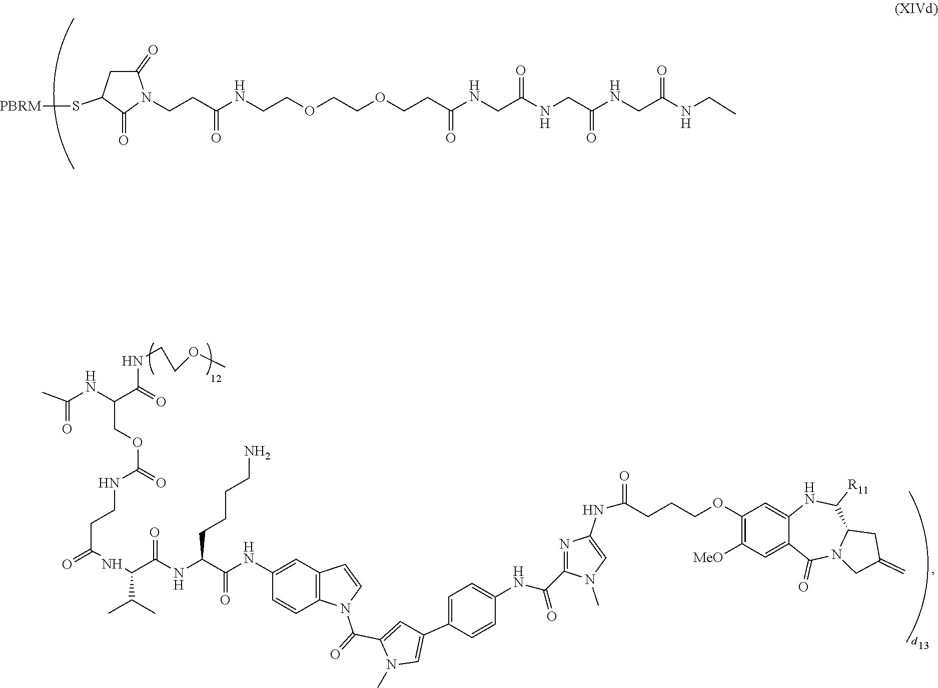

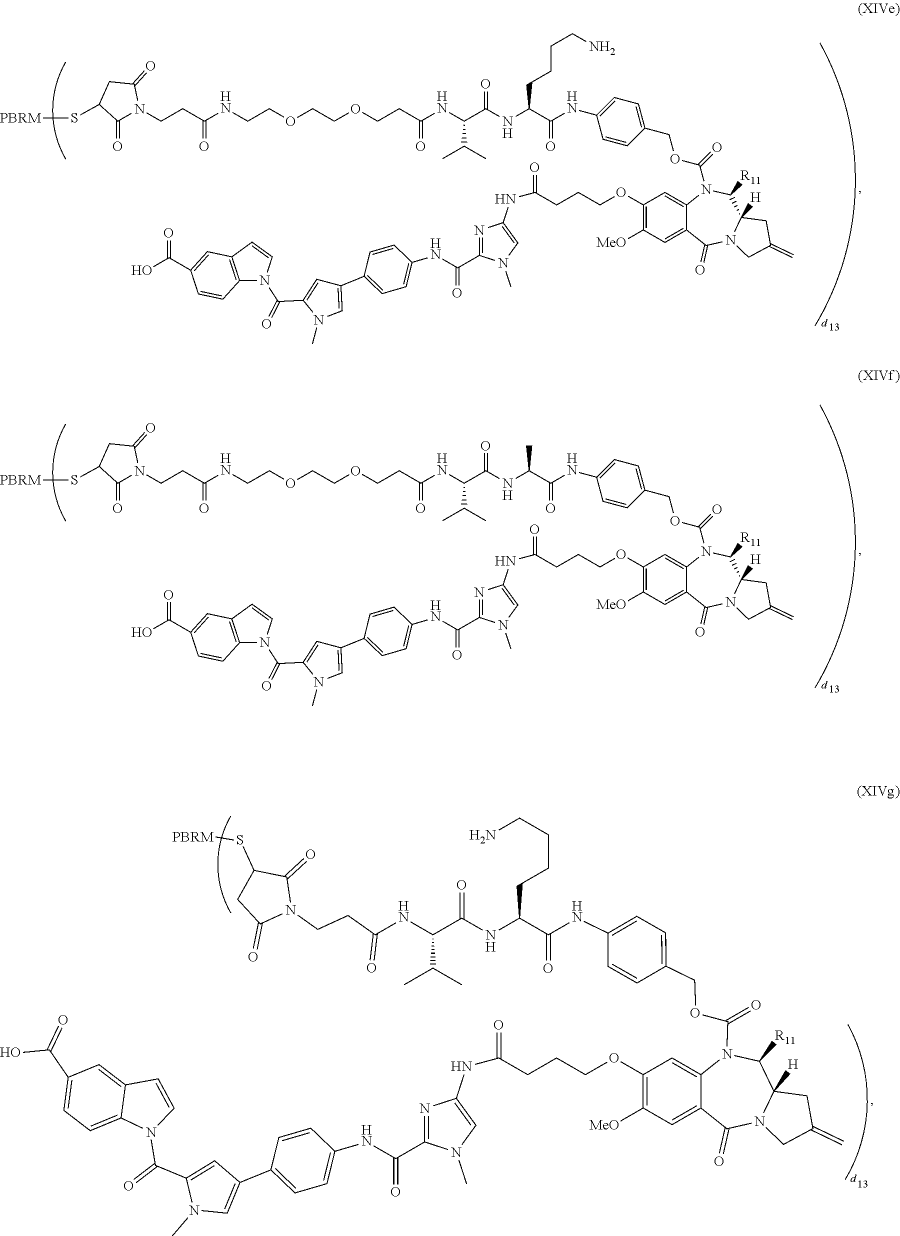

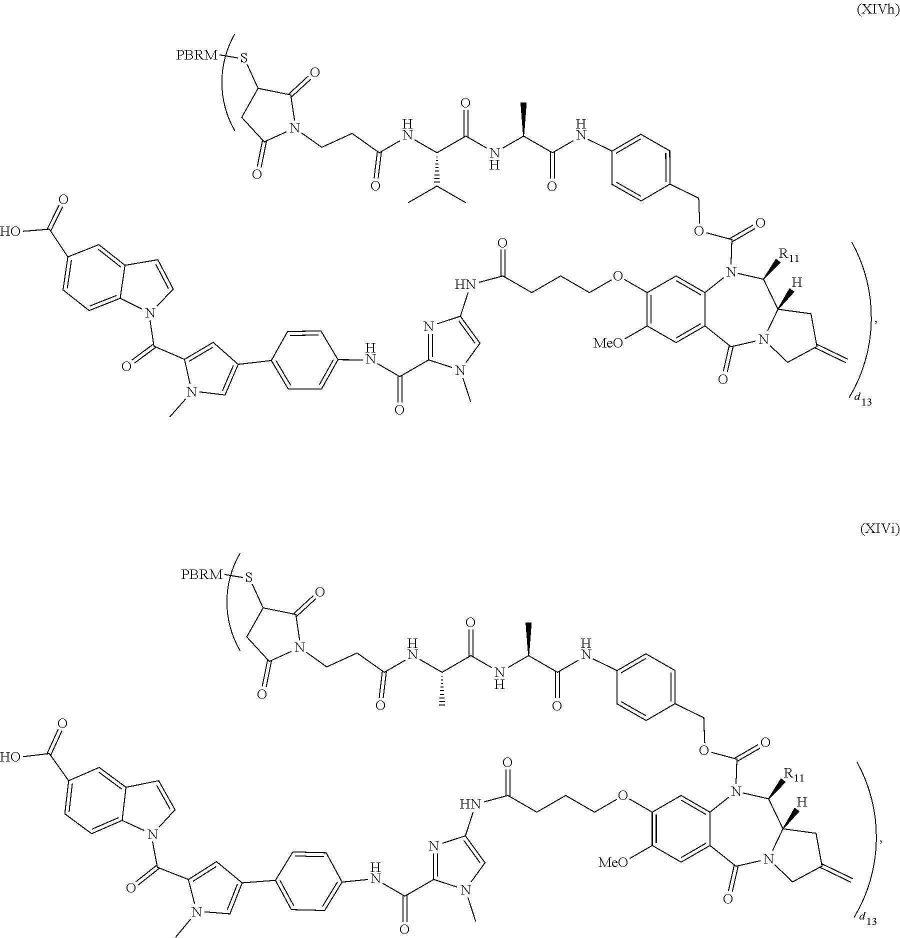

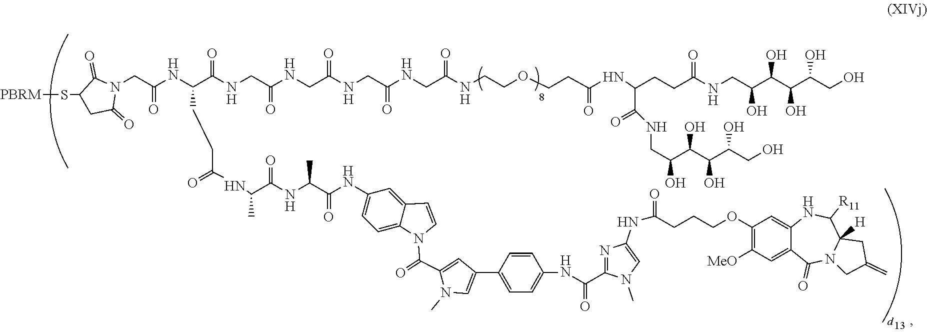

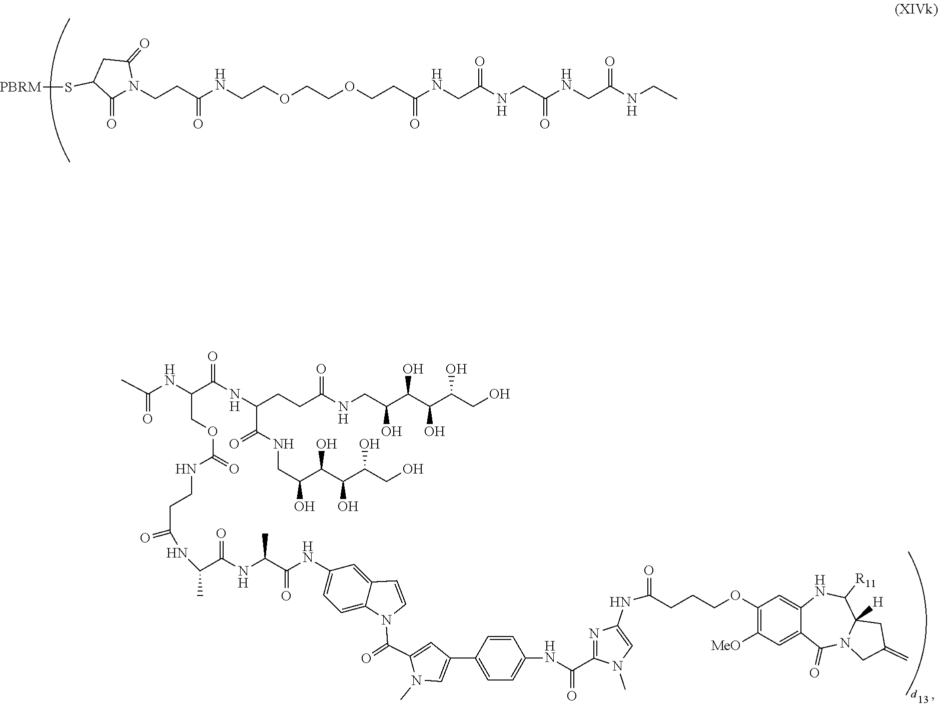

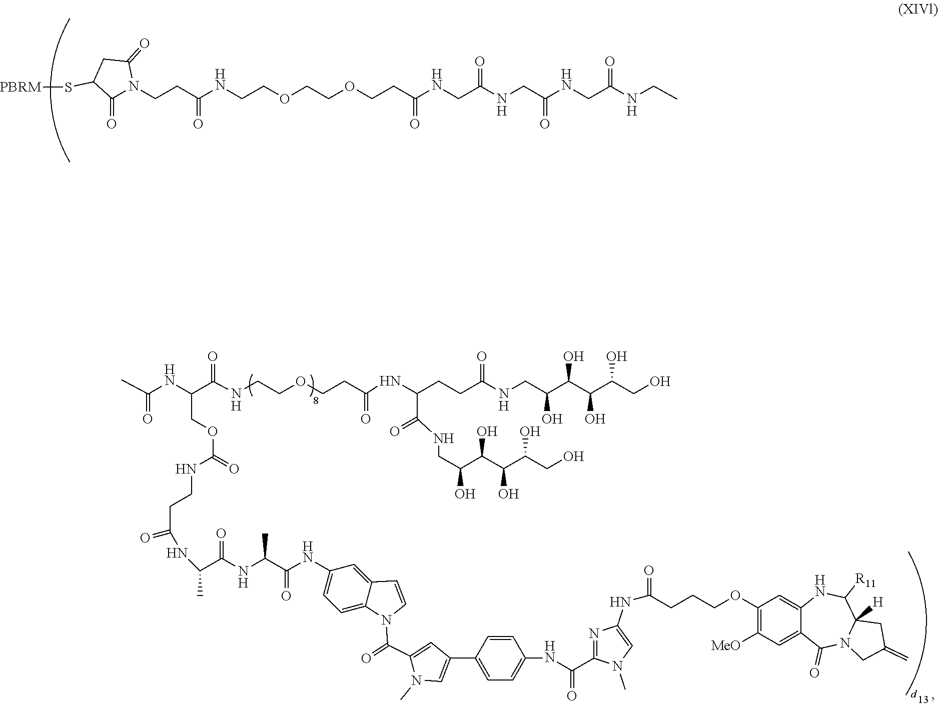

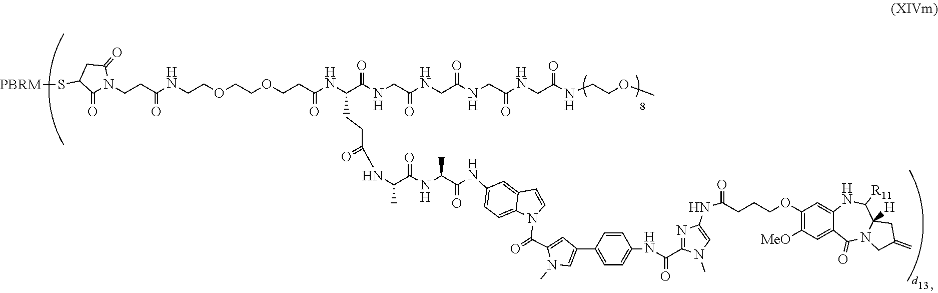

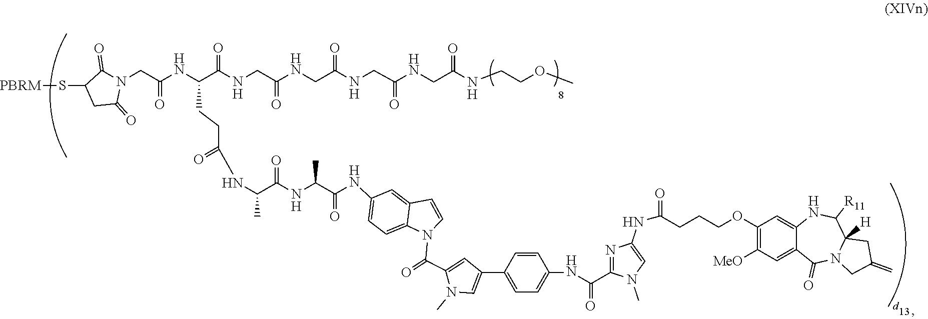

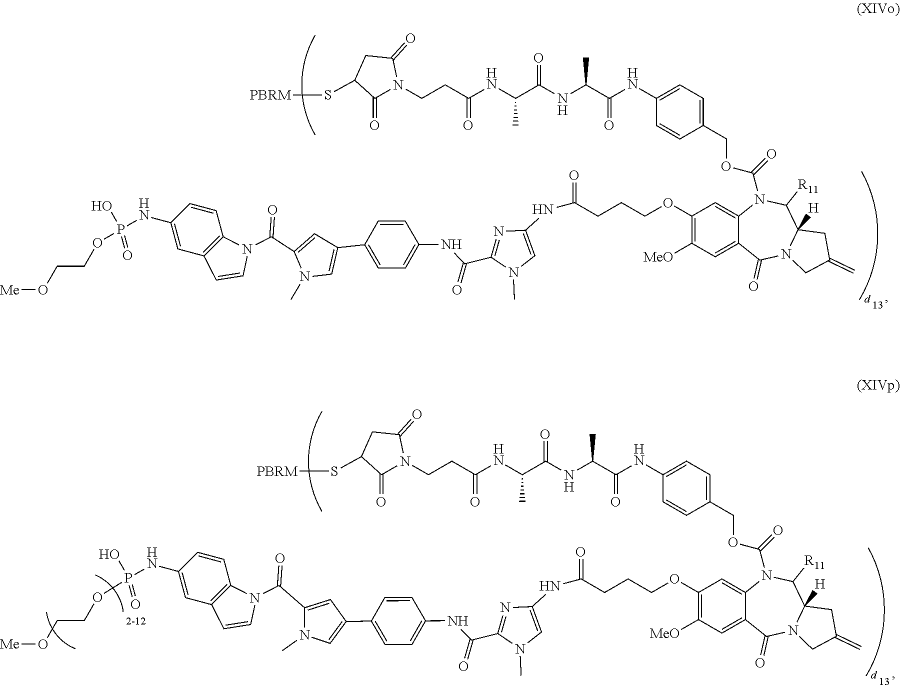

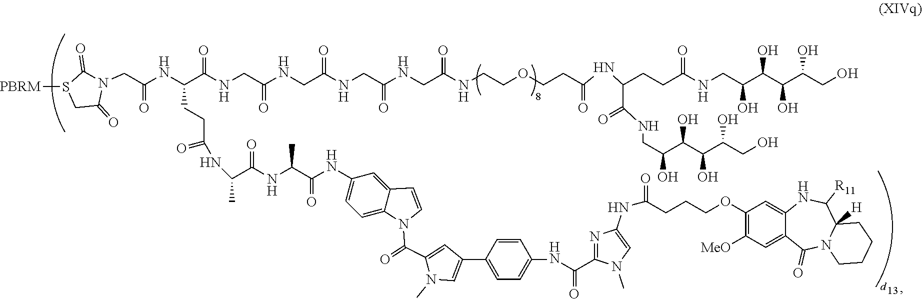

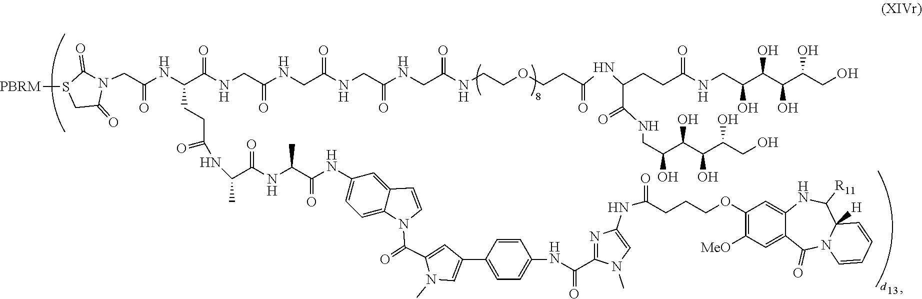

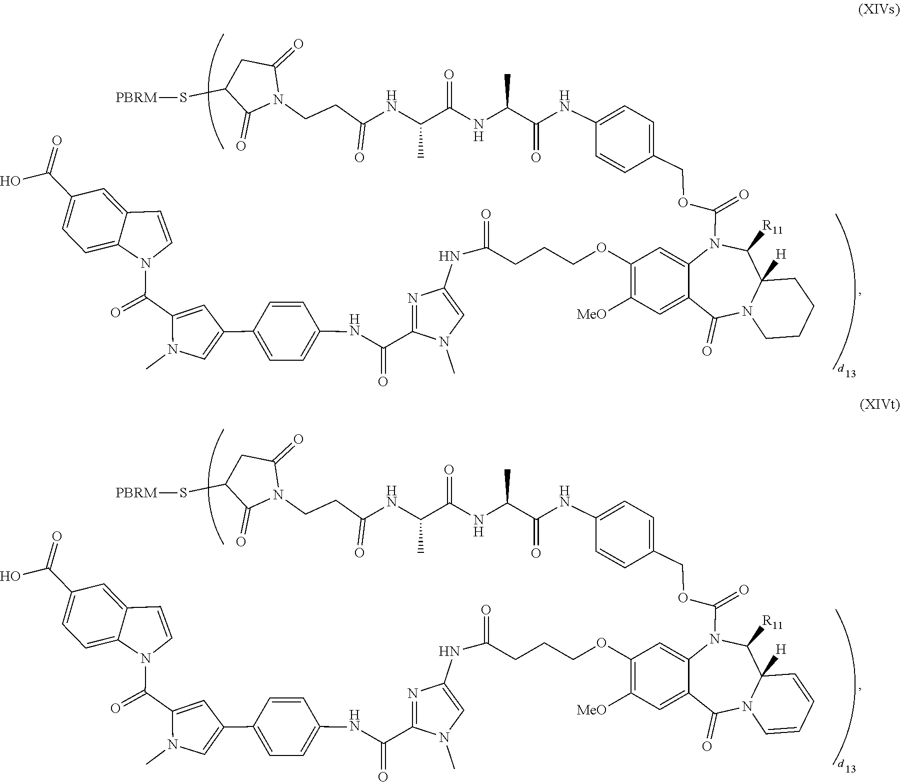

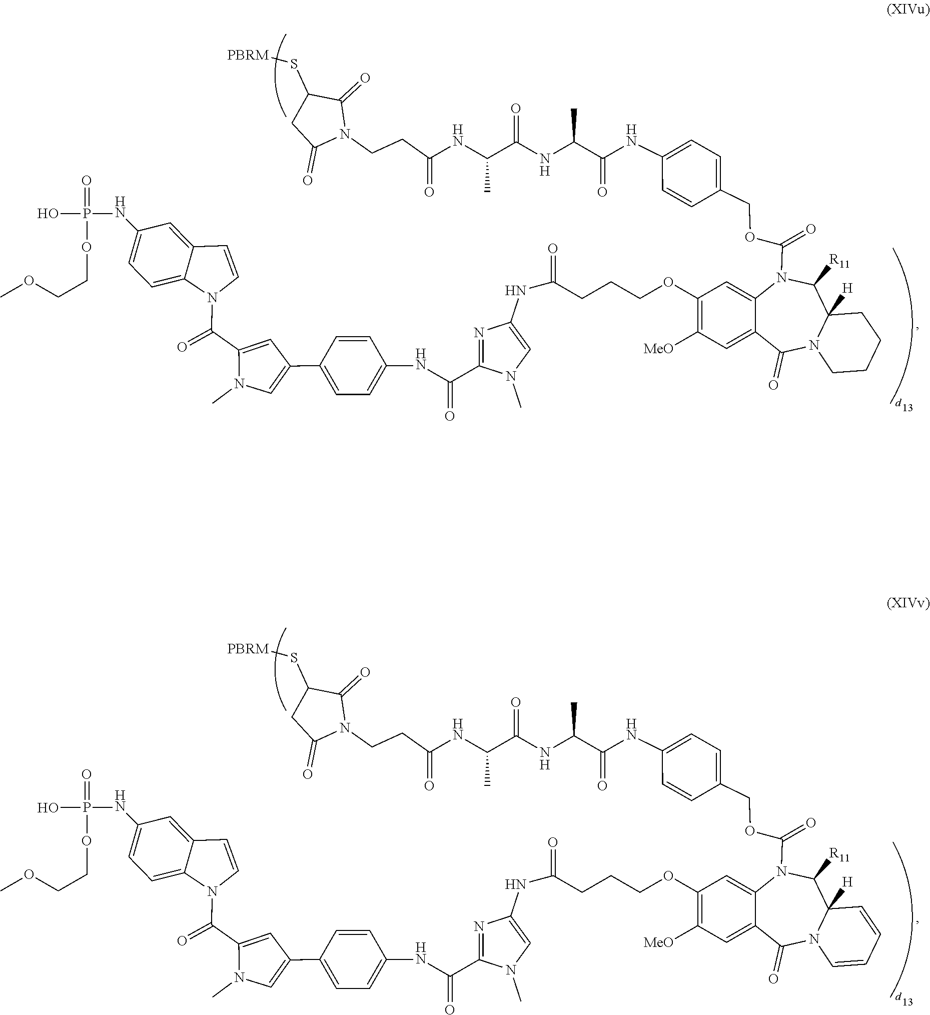

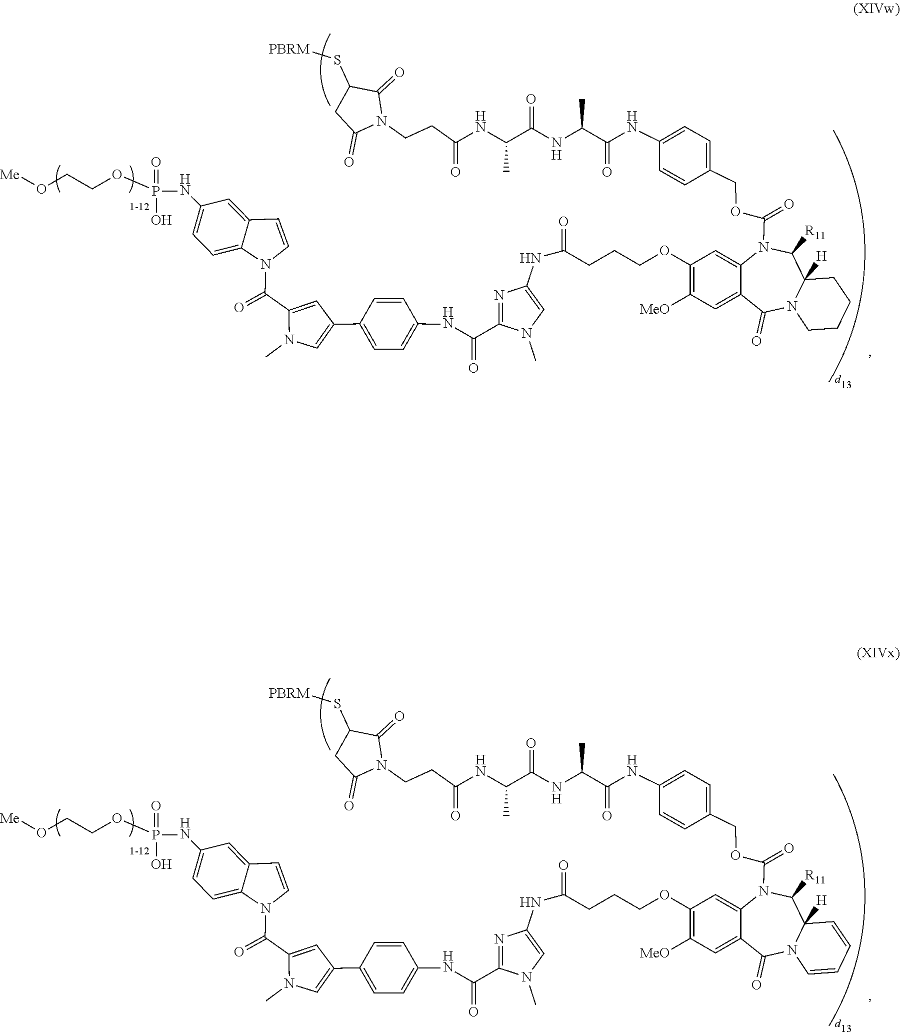

a tautomer thereof, a pharmaceutically acceptable salt or solvate thereof, or a pharmaceutically acceptable salt or solvate of the tautomer, and preferably, d₁₃ is 3 to 5.

In some embodiments, the PBD conjugate is of Formula (XIVa), a tautomer thereof, a pharmaceutically acceptable salt or solvate thereof, or a pharmaceutically acceptable salt or solvate of the tautomer.

In some embodiments, the PBD conjugate is of Formula (XIVa).

In some embodiments, the PBD conjugate is of Formula (XIVb), a tautomer thereof, a pharmaceutically acceptable salt or solvate thereof, or a pharmaceutically acceptable salt or solvate of the tautomer.

In some embodiments, the PBD conjugate is of Formula (XIVb).

In some embodiments, the PBD conjugate is of Formula (XIVc), a tautomer thereof, a pharmaceutically acceptable salt or solvate thereof, or a pharmaceutically acceptable salt or solvate of the tautomer.

In some embodiments, the PBD conjugate is of Formula (XIVc).

In some embodiments, the PBD conjugate is of Formula (XIVd), a tautomer thereof, a pharmaceutically acceptable salt or solvate thereof, or a pharmaceutically acceptable salt or solvate of the tautomer.

In some embodiments, the PBD conjugate is of Formula (XIVd).

In some embodiments, the PBD conjugate is of Formula (XIVe), a tautomer thereof, a pharmaceutically acceptable salt or solvate thereof, or a pharmaceutically acceptable salt or solvate of the tautomer.

In some embodiments, the PBD conjugate is of Formula (XIVe).

In some embodiments, the PBD conjugate is of Formula (XIVf), a tautomer thereof, a pharmaceutically acceptable salt or solvate thereof, or a pharmaceutically acceptable salt or solvate of the tautomer.

In some embodiments, the PBD conjugate is of Formula (XIVf).

In some embodiments, the PBD conjugate is of Formula (XIVg), a tautomer thereof, a pharmaceutically acceptable salt or solvate thereof, or a pharmaceutically acceptable salt or solvate of the tautomer.

In some embodiments, the PBD conjugate is of Formula (XIVg).

In some embodiments, the PBD conjugate is of Formula (XIVh), a tautomer thereof, a pharmaceutically acceptable salt or solvate thereof, or a pharmaceutically acceptable salt or solvate of the tautomer.

In some embodiments, the PBD conjugate is of Formula (XIVh).

In some embodiments, the PBD conjugate is of Formula (XIVi), a tautomer thereof, a pharmaceutically acceptable salt or solvate thereof, or a pharmaceutically acceptable salt or solvate of the tautomer.

In some embodiments, the PBD conjugate is of Formula (XIVi).

In some embodiments, the PBD conjugate is of Formula (XIVj), a tautomer thereof, a pharmaceutically acceptable salt or solvate thereof, or a pharmaceutically acceptable salt or solvate of the tautomer.

In some embodiments, the PBD conjugate is of Formula (XIVj).

In some embodiments, the PBD conjugate is of Formula (XIVk), a tautomer thereof, a pharmaceutically acceptable salt or solvate thereof, or a pharmaceutically acceptable salt or solvate of the tautomer.

In some embodiments, the PBD conjugate is of Formula (XIVk).

In some embodiments, the PBD conjugate is of Formula (XIVl), a tautomer thereof, a pharmaceutically acceptable salt or solvate thereof, or a pharmaceutically acceptable salt or solvate of the tautomer.

In some embodiments, the PBD conjugate is of Formula (XIVl).

In some embodiments, the PBD conjugate is of Formula (XIVm), a tautomer thereof, a pharmaceutically acceptable salt or solvate thereof, or a pharmaceutically acceptable salt or solvate of the tautomer.

In some embodiments, the PBD conjugate is of Formula (XIVm).

In some embodiments, the PBD conjugate is of Formula (XIVn), a tautomer thereof, a pharmaceutically acceptable salt or solvate thereof, or a pharmaceutically acceptable salt or solvate of the tautomer.

In some embodiments, the PBD conjugate is of Formula (XIVn).

In some embodiments, the PBD conjugate is of Formula (XIVo), a tautomer thereof, a pharmaceutically acceptable salt or solvate thereof, or a pharmaceutically acceptable salt or solvate of the tautomer.

In some embodiments, the PBD conjugate is of Formula (XIVo).

In some embodiments, the PBD conjugate is of Formula (XIVp), a tautomer thereof, a pharmaceutically acceptable salt or solvate thereof, or a pharmaceutically acceptable salt or solvate of the tautomer.

In some embodiments, the PBD conjugate is of Formula (XIVp).

In some embodiments, the PBD conjugate is of Formula (XIVq), a tautomer thereof, a pharmaceutically acceptable salt or solvate thereof, or a pharmaceutically acceptable salt or solvate of the tautomer.

In some embodiments, the PBD conjugate is of Formula (XIVq).

In some embodiments, the PBD conjugate is of Formula (XIVr), a tautomer thereof, a pharmaceutically acceptable salt or solvate thereof, or a pharmaceutically acceptable salt or solvate of the tautomer.

In some embodiments, the PBD conjugate is of Formula (XIVr).

In some embodiments, the PBD conjugate is of Formula (XIVs), a tautomer thereof, a pharmaceutically acceptable salt or solvate thereof, or a pharmaceutically acceptable salt or solvate of the tautomer.

In some embodiments, the PBD conjugate is of Formula (XIVs).

In some embodiments, the PBD conjugate is of Formula (XIVt), a tautomer thereof, a pharmaceutically acceptable salt or solvate thereof, or a pharmaceutically acceptable salt or solvate of the tautomer.

In some embodiments, the PBD conjugate is of Formula (XIVt).

In some embodiments, the PBD conjugate is of Formula (XIVu), a tautomer thereof, a pharmaceutically acceptable salt or solvate thereof, or a pharmaceutically acceptable salt or solvate of the tautomer.

In some embodiments, the PBD conjugate is of Formula (XIVu).

In some embodiments, the PBD conjugate is of Formula (XIVv), a tautomer thereof, a pharmaceutically acceptable salt or solvate thereof, or a pharmaceutically acceptable salt or solvate of the tautomer.

In some embodiments, the PBD conjugate is of Formula (XIVv).

In some embodiments, the PBD conjugate is of Formula (XIVw), a tautomer thereof, a pharmaceutically acceptable salt or solvate thereof, or a pharmaceutically acceptable salt or solvate of the tautomer.

In some embodiments, the PBD conjugate is of Formula (XIVw).

In some embodiments, the PBD conjugate is of Formula (XIVx), a tautomer thereof, a pharmaceutically acceptable salt or solvate thereof, or a pharmaceutically acceptable salt or solvate of the tautomer.

In some embodiments, the PBD conjugate is of Formula (XIVx).

In some embodiment the PBD drug moiety (D) of the PBD conjugate exhibits bystander killing effects. In these embodiments the PBD drug moiety is highly membrane-permeable whereas its hydrolysis products has a low level of permeability and is locked in the cell.

In some embodiment the PBD drug moiety (D) of the PBD conjugate is not a substract for P-gp efflux pumps.

Pharmaceutical Compositions

Also included are pharmaceutical compositions comprising one or more conjugates as disclosed herein in an acceptable carrier, such as a stabilizer, buffer, and the like. The conjugates can be administered and introduced into a subject by standard means, with or without stabilizers, buffers, and the like, to form a pharmaceutical composition. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal, oral or parenteral administration including intravenous, intraarterial, subcutaneous, intraperitoneal or intramuscular injection or infusion or intracranial, e.g., intrathecal or intraventricular, administration. The conjugates can be formulated and used as sterile solutions and/or suspensions for injectable administration; lyophilized powders for reconstitution prior to injection/infusion; topical compositions; as tablets, capsules, or elixirs for oral administration; or suppositories for rectal administration, and the other compositions known in the art.

The pharmaceutical compositions of the conjugates described herein can be included in a container, pack, or dispenser together with instructions for administration.

In some embodiments, the compositions can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

In some embodiments, the active compounds (e.g., conjugates or drugs of the disclosure) are administered in combination therapy, i.e., combined with other agents, e.g., therapeutic agents, that are useful for treating pathological conditions or disorders, such as various forms of cancer, autoimmune disorders and inflammatory diseases. The term “in combination” in this context means that the agents are given substantially contemporaneously, either simultaneously or sequentially. If given sequentially, at the onset of administration of the second compound, the first of the two compounds is preferably still detectable at effective concentrations at the site of treatment.

In some embodiments, the combination therapy can include one or more conjugates disclosed herein coformulated with, and/or coadministered with, one or more additional antibodies, which can be the same as the antibody used to form the conjugate or a different antibody.

In some embodiments, the combination therapy can include one or more therapeutic agent and/or adjuvant. In certain embodiments, the additional therapeutic agent is a small molecule inhibitor, another antibody-based therapy, a polypeptide or peptide-based therapy, a nucleic acid-based therapy and/or other biologic.

In certain embodiments, the additional therapeutic agent is a cytotoxic agent, a chemotherapeutic agent, a growth inhibitory agent, an angiogenesis inhibitor, a PARP (poly(ADP)-ribose polymerase) inhibitor, an alkylating agent, an anti-metabolite, an anti-microtubule agent, a topoisomerase inhibitor, a cytotoxic antibiotic, any other nucleic acid damaging agent or an immune checkpoint inhibitor. In one embodiment, the therapeutic agent used in the treatment of cancer, includes but is not limited to, a platinum compound (e.g., cisplatin or carboplatin); a taxane (e.g., paclitaxel or docetaxel); a topoisomerase inhibitor (e.g., irinotecan or topotecan); an anthracycline (e.g., doxorubicin (ADRIAMYCIN®) or liposomal doxorubicin (DOXIL®)); an anti-metabolite (e.g., gemcitabine, pemetrexed); cyclophosphamide; vinorelbine (NAVELBINE®); hexamethylmelamine; ifosfamide; etoposide; an angiogenesis inhibitor (e.g., Bevacizumab (Avastin®)), thalidomide, TNP-470, platelet factor 4, interferon or endostatin); a PARP inhibitor (e.g., Olaparib (Lynparza™)); an immune checkpoint inhibitor, such as for example, a monoclonal antibody that targets either PD-1 or PD-L ((Pembrolizumab (Keytruda®), atezolizumab (MPDL3280A) or Nivolumab (Opdivo®)) or CTA-4 (Ipilimumab (Yervoy®), a kinase inhibitor (e.g., sorafenib or erlotinib), a proteasome inhibitor (e.g., bortezomib or carfilzomib), an immune modulating agent (e.g., lenalidomide or IL-2), a radiation agent, an ALK inhibitor (e.g. crizotinib (Xalkori), ceritinib (Zykadia), alectinib (Alecensa), dalantercept (ACE-041), brigatinib (AP26113), entrectinib (NMS-E628), PF-06463922 TSR-011, CEP-37440 and X-396) and/or a biosimilar thereof and/or combinations thereof. Other suitable agents include an agent considered standard of care by those skilled in the art and/or a chemotherapeutic agent well known to those skilled in the art.

In some embodiments, the immune checkpoint inhibitor is an inhibitor of CTLA-4. In some embodiments, the immune checkpoint inhibitor is an antibody against CTLA-4. In some embodiments, the immune checkpoint inhibitor is a monoclonal antibody against CTLA-4. In other embodiments, the immune checkpoint inhibitor is a human or humanized antibody against CTLA-4. In one embodiment, the anti-CTLA-4 antibody blocks the binding of CTLA-4 to CD80 (B7-1) and/or CD86 (B7-2) expressed on antigen presenting cells. Exemplary antibodies against CTLA-4 include, but are not limited to, Bristol Meyers Squibb's anti-CTLA-4 antibody ipilimumab (also known as Yervoy®, MDX-010, BMS—734016 and MDX-101); anti-CTLA4 Antibody, clone 9H10 from Millipore; Pfizer's tremelimumab (CP-675,206, ticilimumab); and anti-CTLA4 antibody clone BNI3 from Abcam.

In some embodiments, the anti-CTLA-4 antibody is an anti-CTLA-4 antibody disclosed in any of the following patent publications (herein incorporated by reference): WO 2001014424; WO 2004035607; US2005/0201994; EP 1212422 B1; WO2003086459; WO2012120125; WO2000037504; WO2009100140; W0200609649; WO2005092380; WO2007123737; WO2006029219; WO20100979597; W0200612168; and WO1997020574. Additional CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014; and/or U.S. Pat. Nos. 5,977,318, 6,682,736, 7,109,003, and 7,132,281, incorporated herein by reference). In some embodiments, the anti-CTLA-4 antibody is for example, those disclosed in: WO 98/42752; U.S. Pat. Nos. 6,682,736 and 6,207,156; Hurwitz et al, Proc. Nat. Acad. Sci. USA, 95(17): 10067-10071 (1998); Camacho et al, J. Clin. Oncol., 22(145): Abstract No. 2505 (2004) (antibody CP-675206); Mokyr et al, Cancer Res., 58:5301-5304 (1998) (incorporated herein by reference).

In some embodiments, the CTLA-4 inhibitor is a CTLA-4 ligand as disclosed in WO1996040915.

In some embodiments, the CTLA-4 inhibitor is a nucleic acid inhibitor of CTLA-4 expression. In some embodiments, anti-CTLA4 RNAi molecules may take the form of the molecules described by Mello and Fire in PCT Publication Nos. WO 1999/032619 and WO 2001/029058; U.S. Publication Nos. 2003/0051263, 2003/0055020, 2003/0056235, 2004/265839, 2005/0100913, 2006/0024798, 2008/0050342, 2008/0081373, 2008/0248576, and 2008/055443; and/or U.S. Pat. Nos. 6,506,559, 7,282,564, 7,538,095, and 7,560,438 (incorporated herein by reference). In some instances, the anti-CTLA4 RNAi molecules take the form of double stranded RNAi molecules described by Tuschl in European Patent No. EP 1309726 (incorporated herein by reference). In some instances, the anti-CTLA4 RNAi molecules take the form of double stranded RNAi molecules described by Tuschl in U.S. Pat. Nos. 7,056,704 and 7,078,196 (incorporated herein by reference). In some embodiments, the CTLA4 inhibitor is an aptamer described in PCT Publication No. WO2004081021.

Additionally, the anti-CTLA4 RNAi molecules of the present disclosure may take the form be RNA molecules described by Crooke in U.S. Pat. Nos. 5,898,031, 6,107,094, 7,432,249, and 7,432,250, and European Application No. EP 0928290 (incorporated herein by reference).

In some embodiments, the immune checkpoint inhibitor is an inhibitor of PD-L1. In some embodiments, the immune checkpoint inhibitor is an antibody against PD-L1. In some embodiments, the immune checkpoint inhibitor is a monoclonal antibody against PD-L1. In other or additional embodiments, the immune checkpoint inhibitor is a human or humanized antibody against PD-L1. In one embodiment, the immune checkpoint inhibitor reduces the expression or activity of one or more immune checkpoint proteins, such as PD-L1. In another embodiment, the immune checkpoint inhibitor reduces the interaction between PD-1 and PD-L1. Exemplary immune checkpoint inhibitors include antibodies (e.g., an anti-PD-L1 antibody), RNAi molecules (e.g., anti-PD-L1 RNAi), antisense molecules (e.g., an anti-PD-L¹ antisense RNA), dominant negative proteins (e.g., a dominant negative PD-L1 protein), and small molecule inhibitors. Antibodies include monoclonal antibodies, humanized antibodies, deimmunized antibodies, and Ig fusion proteins. An exemplary anti-PD-L1 antibody includes clone EH12. Exemplary antibodies against PD-L1 include: Genentech's MPDL3280A (RG7446); Anti-mouse PD-L1 antibody Clone 10F.9G2 (Cat #BE0101) from BioXcell; anti-PD-L1 monoclonal antibody MDX-1105 (BMS—936559) and BMS—935559 from Bristol-Meyer's Squibb; MSB0010718C; mouse anti-PD-L1 Clone 29E.2A3; and AstraZeneca's MED14736. In some embodiments, the anti-PD-L1 antibody is an anti-PD-L1 antibody disclosed in any of the following patent publications (herein incorporated by reference): WO2013079174; CN101104640; WO2010036959; WO2013056716; WO2007005874; WO2010089411; WO2010077634; WO2004004771; WO2006133396; W0201309906; US 20140294898; WO2013181634 or WO2012145493.

In some embodiments, the PD-L1 inhibitor is a nucleic acid inhibitor of PD-L₁ expression. In some embodiments, the PD-L1 inhibitor is disclosed in one of the following patent publications (incorporated herein by reference): WO2011127180 or WO2011000841. In some embodiments, the PD-L1 inhibitor is rapamycin.

In some embodiments, the immune checkpoint inhibitor is an inhibitor of PD-L2. In some embodiments, the immune checkpoint inhibitor is an antibody against PD-L2. In some embodiments, the immune checkpoint inhibitor is a monoclonal antibody against PD-L2. In other or additional embodiments, the immune checkpoint inhibitor is a human or humanized antibody against PD-L2. In some embodiments, the immune checkpoint inhibitor reduces the expression or activity of one or more immune checkpoint proteins, such as PD-L2. In other embodiments, the immune checkpoint inhibitor reduces the interaction between PD-1 and PD-L2. Exemplary immune checkpoint inhibitors include antibodies (e.g., an anti-PD-L2 antibody), RNAi molecules (e.g., an anti-PD-L2 RNAi), antisense molecules (e.g., an anti-PD-L2 antisense RNA), dominant negative proteins (e.g., a dominant negative PD-L2 protein), and small molecule inhibitors. Antibodies include monoclonal antibodies, humanized antibodies, deimmunized antibodies, and Ig fusion proteins.

In some embodiments, the PD-L2 inhibitor is GlaxoSmithKline's AMP-224 (Amplimmune). In some embodiments, the PD-L2 inhibitor is rHIgM12B7.

In some embodiments, the immune checkpoint inhibitor is an inhibitor of PD-L1. In some embodiments, the immune checkpoint inhibitor is an antibody against PD-1. In some embodiments, the immune checkpoint inhibitor is a monoclonal antibody against PD-1. In other embodiments, the immune checkpoint inhibitor is a human or humanized antibody against PD-1. In some embodiments, the inhibitors of PD-1 biological activity (or its ligands) disclosed in U.S. Pat. Nos. 7,029,674; 6,808,710; or U.S. Patent Application Nos: 20050250106 and 20050159351 can be used in the combinations provided herein. Exemplary antibodies against PD-1 include: Anti-mouse PD-1 antibody Clone J43 (Cat #BE0033-2) from BioXcell; Anti-mouse PD-1 antibody Clone RMP1-14 (Cat #BE0146) from BioXcell; mouse anti-PD-1 antibody Clone EH12; Merck's MK-3475 anti-mouse PD-1 antibody (Keytruda®, pembrolizumab, lambrolizumab, h409A1 1); and AnaptysBio's anti-PD-1 antibody, known as ANBO1; antibody MDX-1 106 (ONO—4538); Bristol-Myers Squibb's human IgG4 monoclonal antibody nivolumab (Opdivo®, BMS—936558, MDX1106); AstraZeneca's AMP-514, and AMP-224; and Pidilizumab (CT-011 or hBAT-1), CureTech Ltd.

Additional exemplary anti-PD-1 antibodies are described by Goldberg et al, Blood 1 10(1): 186-192 (2007), Thompson et al, Clin. Cancer Res. 13(6): 1757-1761 (2007), and Korman et al, International Application No. PCT/JP2006/309606 (publication no. WO 2006/121168 A1), each of which are expressly incorporated by reference herein. In some embodiments, the anti-PD-antibody is an anti-PD-1 antibody disclosed in any of the following patent publications (herein incorporated by reference): W0014557; WO2011110604; WO2008156712; US2012023752; WO2011110621; WO2004072286; WO2004056875; WO20100036959; WO2010029434; W0201213548; WO2002078731; WO2012145493; WO2010089411; WO2001014557; WO2013022091; WO2013019906; WO2003011911; US20140294898; and WO2010001617.

In some embodiments, the PD-1 inhibitor is a PD-1 binding protein as disclosed in W0200914335 (herein incorporated by reference).

In some embodiments, the PD-1 inhibitor is a peptidomimetic inhibitor of PD-as disclosed in WO2013132317 (herein incorporated by reference).

In some embodiments, the PD-1 inhibitor is an anti-mouse PD-mAb: clone J43, BioXCell (West Lebanon, N.H.).

In some embodiments, the PD-inhibitor is a PD-L1 protein, a PD-L2 protein, or fragments, as well as antibody MDX-1 106 (ONO—4538) tested in clinical studies for the treatment of certain malignancies (Brahmer et al., J Clin Oncol. 2010 28(19): 3167-75, Epub 2010 Jun. 1). Other blocking antibodies may be readily identified and prepared by the skilled person based on the known domain of interaction between PD- and PD-L1/PD-L2, as discussed above. In some embodiments, a peptide corresponding to the IgV region of PD- or PD-L1/PD-L₂ (or to a portion of this region) could be used as an antigen to develop blocking antibodies using methods well known in the art.

In some embodiments, the immune checkpoint inhibitor is an inhibitor of IDO1. In some embodiments, the immune checkpoint inhibitor is a small molecule against IDO1. Exemplary small molecules against IDO1 include: Incyte's INCB024360, NSC1-721782 (also known as 1-methyl-D-tryptophan), and Bristol Meyers Squibb's F001287.

In some embodiments, the immune checkpoint inhibitor is an inhibitor of LAG3 (CD223). In some embodiments, the immune checkpoint inhibitor is an antibody against LAG3. In some embodiments, the immune checkpoint inhibitor is a monoclonal antibody against LAG3. In other or additional embodiments, the immune checkpoint inhibitor is a human or humanized antibody against LAG3. In additional embodiments, an antibody against LAG3 blocks the interaction of LAG3 with major histocompatibility complex (MHC) class H molecules. Exemplary antibodies against LAG3 include: anti-Lag-3 antibody clone eBioC9B7W (C9B7W) from eBioscience; anti-Lag3 antibody LS-B2237 from LifeSpan Biosciences; IMP 321 (ImmuFact) from Immutep; anti-Lag3 antibody BMS—986016; and the LAG-3 chimeric antibody A9H12. In some embodiments, the anti-LAG3 antibody is an anti-LAG3 antibody disclosed in any of the following patent publications (herein incorporated by reference): WO2010019570; WO2008132601; or WO2004078928.

In some embodiments, the immune checkpoint inhibitor is an antibody against TIM3 (also known as HAVCR2). In some embodiments, the immune checkpoint inhibitor is a monoclonal antibody against TIM3. In other or additional embodiments, the immune checkpoint inhibitor is a human or humanized antibody against TIM3. In additional embodiments, an antibody against TIM3 blocks the interaction of TIM3 with galectin-9 (Gal9). In some embodiments, the anti-TIM3 antibody is an anti-TIM3 antibody disclosed in any of the following patent publications (herein incorporated by reference): WO2013006490; W0201155607; WO2011159877; or W0200117057. In another embodiment, a TIM3 inhibitor is a TIM3 inhibitor disclosed in WO2009052623.

In some embodiments, the immune checkpoint inhibitor is an antibody against B7-H3. In one embodiment, the immune checkpoint inhibitor is MGA271.

In some embodiments, the immune checkpoint inhibitor is an antibody against MR. In one embodiment, the immune checkpoint inhibitor is Lirilumab (IPH2101). In some embodiments, an antibody against MR blocks the interaction of KIR with HLA.

In some embodiments, the immune checkpoint inhibitor is an antibody against CD137 (also known as 4-1BB or TNFRSF9). In one embodiment, the immune checkpoint inhibitor is urelumab (BMS—663513, Bristol-Myers Squibb), PF-05082566 (anti-4-1BB, PF-2566, Pfizer), or XmAb-5592 (Xencor). In one embodiment, an anti-CD137 antibody is an antibody disclosed in U.S. Published Application No. US 2005/0095244; an antibody disclosed in issued U.S. Pat. No. 7,288,638 (such as 20H4.9-IgG4 [1007 or BMS—663513] or 20H4.9-IgG1 [BMS—663031]); an antibody disclosed in issued U.S. Pat. No. 6,887,673 [4E9 or BMS—554271]; an antibody disclosed in issued U.S. Pat. No. 7,214,493; an antibody disclosed in issued U.S. Pat. No. 6,303,121; an antibody disclosed in issued U.S. Pat. No. 6,569,997; an antibody disclosed in issued U.S. Pat. No. 6,905,685; an antibody disclosed in issued U.S. Pat. No. 6,355,476; an antibody disclosed in issued U.S. Pat. No. 6,362,325 [1D8 or BMS—469492; 3H3 or BMS—469497; or 3E1]; an antibody disclosed in issued U.S. Pat. No. 6,974,863 (such as 53A2); or an antibody disclosed in issued U.S. Pat. No. 6,210,669 (such as 1D8, 3B8, or 3E1). In a further embodiment, the immune checkpoint inhibitor is one disclosed in WO 2014036412. In another embodiment, an antibody against CD137 blocks the interaction of CD137 with CD137L.

In some embodiments, the immune checkpoint inhibitor is an antibody against PS. In one embodiment, the immune checkpoint inhibitor is Bavituximab.

In some embodiments, the immune checkpoint inhibitor is an antibody against CD52. In one embodiment, the immune checkpoint inhibitor is alemtuzumab.

In some embodiments, the immune checkpoint inhibitor is an antibody against CD30. In one embodiment, the immune checkpoint inhibitor is brentuximab vedotin. In another embodiment, an antibody against CD30 blocks the interaction of CD30 with CD30L.

In some embodiments, the immune checkpoint inhibitor is an antibody against CD33. In one embodiment, the immune checkpoint inhibitor is gemtuzumab ozogamicin.

In some embodiments, the immune checkpoint inhibitor is an antibody against CD20. In one embodiment, the immune checkpoint inhibitor is ibritumomab tiuxetan. In another embodiment, the immune checkpoint inhibitor is ofatumumab. In another embodiment, the immune checkpoint inhibitor is rituximab. In another embodiment, the immune checkpoint inhibitor is tositumomab.

In some embodiments, the immune checkpoint inhibitor is an antibody against CD27 (also known as TNFRSF7). In one embodiment, the immune checkpoint inhibitor is CDX-1127 (Celldex Therapeutics). In another embodiment, an antibody against CD27 blocks the interaction of CD27 with CD70.

In some embodiments, the immune checkpoint inhibitor is an antibody against OX40 (also known as TNFRSF4 or CD134). In one embodiment, the immune checkpoint inhibitor is anti-OX40 mouse IgG. In another embodiment, an antibody against 0×40 blocks the interaction of OX40 with OX40L.

In some embodiments, the immune checkpoint inhibitor is an antibody against glucocorticoid-induced tumor necrosis factor receptor (GITR). In one embodiment, the immune checkpoint inhibitor is TRX518 (GITR, Inc.). In another embodiment, an antibody against GITR blocks the interaction of GITR with GITRL.

In some embodiments, the immune checkpoint inhibitor is an antibody against inducible T-cell COStimulator (ICOS, also known as CD278). In one embodiment, the immune checkpoint inhibitor is MEDI570 (MedImmune, LLC) or AMG557 (Amgen). In another embodiment, an antibody against ICOS blocks the interaction of ICOS with ICOSL and/or B7-H2.

In some embodiments, the immune checkpoint inhibitor is an inhibitor against BTLA (CD272), CD160, 2B4, LAIR1, TIGHT, LIGHT, DR3, CD226, CD2, or SLAM. As described elsewhere herein, an immune checkpoint inhibitor can be one or more binding proteins, antibodies (or fragments or variants thereof) that bind to immune checkpoint molecules, nucleic acids that downregulate expression of the immune checkpoint molecules, or any other molecules that bind to immune checkpoint molecules (i.e. small organic molecules, peptidomimetics, aptamers, etc.). In some instances, an inhibitor of BTLA (CD272) is HVEM. In some instances, an inhibitor of CD160 is HVEM. In some cases, an inhibitor of 2B4 is CD48. In some instances, an inhibitor of LAIR1 is collagen. In some instances, an inhibitor of TIGHT is CD112, CD13, or CD155. In some instances, an inhibitor of CD28 is CD80 or CD86. In some instances, an inhibitor of LIGHT is HVEM. In some instances, an inhibitor of DR3 is TL1A. In some instances, an inhibitor of CD226 is CD155 or CD112. In some cases, an inhibitor of CD2 is CD48 or CD58. In some cases, SLAM is self-inhibitory and an inhibitor of SLAM is SLAM.

In certain embodiments, the immune checkpoint inhibitor inhibits a checkpoint protein that include, but are not limited to CTLA4 (cytotoxic T-lymphocyte antigen 4, also known as CD152), PD-L1 (programmed cell death 1 ligand 1, also known as CD274), PDL2 programmed cell death protein 2), PD-1 (programmed cell death protein 1, also known as CD279), a B-7 family ligand (B7-H1, B7-H3, B7-H4) BTLA (B and T lymphocyte attenuator, also known as CD272), HVEM, TIM3 (T-cell membrane protein 3), GAL9, LAG-3 (lymphocyte activation gene-3; CD223), VISTA, KIR (killer immunoglobulin receptor), 2B4 (also known as CD244), CD160, CGEN-15049, CHK1 (Checkpoint kinase 1), CHK2 (Checkpoint kinase 2), A2aR (adenosine A2a receptor), CD2, CD27, CD28, CD30, CD40, CD70, CD80, CD86, CD137, CD226, CD276, DR3, GITR, HAVCR2, HVEM, IDO1 (indoleamine 2,3-dioxygenase 1), IDO2 (indoleamine 2,3-dioxygenase 2), ICOS (inducible T cell costimulator), LAIR1, LIGHT (also known as TNFSF14, a TNF family member), MARCO (macrophage receptor with collagenous structure), OX40 (also known as tumor necrosis factor receptor superfamily, member 4, TNFRSF4, and CD134) and its ligand OX40L (CD252), SLAM, TIGHT, VTCN1 or a combination thereof.

In certain embodiments, the immune checkpoint inhibitor interacts with a ligand of a checkpoint protein that comprises CTLA-4, PDL1, PDL2, PD1, BTLA, HVEM, TIM3, GAL9, LAG3, VISTA, KIR, 2B4, CD160, CGEN-15049, CHK1, CHK2, A2aR, a B-7 family ligand, CD2, CD27, CD28, CD30, CD40, CD70, CD80, CD86, CD137, CD226, CD276, DR3, GITR, HAVCR2, HVEM, IDO1, IDO2, ICOS (inducible T cell costimulator), LAIR1, LIGHT, MARCO (macrophage receptor with collagenous structure), OX-40, SLAM, TIGHT, VTCN1 or a combination thereof.

In certain embodiments, the immune checkpoint inhibitor inhibits a checkpoint protein that comprises CTLA-4, PDL1, PD1 or a combination thereof.

In certain embodiments, the immune checkpoint inhibitor inhibits a checkpoint protein that comprises CTLA-4 and PD1 or a combination thereof.

In certain embodiments, the immune checkpoint inhibitor comprises pembrolizumab (MK-3475), nivolumab (BMS—936558), pidilizumab (CT-011), AMP-224, MDX-1 105, durvalumab (MEDI4736), MPDL3280A, BMS—936559, IPH2101, TSR-042, TSR-022, ipilimumab, lirilumab, atezolizumab, avelumab, tremelimumab, or a combination thereof.

In certain embodiments, the immune checkpoint inhibitor is nivolumab (BMS—936558), ipilimumab, pembrolizumab, atezolizumab, tremelimumab, durvalumab, avelumab, or a combination thereof.

In certain embodiments, the immune checkpoint inhibitor is pembrolizumab.

A pharmacological composition or formulation refers to a composition or formulation in a form suitable for administration, e.g., systemic administration, into a cell or subject, including for example a human. Suitable forms, in part, depend upon the use or the route of entry, for example oral, inhaled, transdermal, or by injection/infusion. Such forms should not prevent the composition or formulation from reaching a target cell (i.e., a cell to which the drug is desirable for delivery). In some embodiments, pharmacological compositions injected into the blood stream should be soluble. Other factors are known in the art, and include considerations such as toxicity and forms that prevent the composition or formulation from exerting its effect.

By “systemic administration” is meant in vivo systemic absorption or accumulation of the conjugate in the blood stream followed by distribution throughout the entire body. Administration routes that lead to systemic absorption include, without limitation: intravenous, subcutaneous, intraperitoneal, inhalation, oral, intrapulmonary, and intramuscular. Each of these administration routes exposes the conjugates to an accessible diseased tissue. The rate of entry of an active agent into the circulation has been shown to be a function of molecular weight or size. The use of a conjugate of this disclosure can localize the drug delivery in certain cells, such as cancer cells via the specificity of PBRMs.

A “pharmaceutically acceptable formulation” means a composition or formulation that allows for the effective distribution of the conjugates in the physical location most suitable for their desired activity. In one embodiment, effective delivery occurs before clearance by the reticuloendothelial system or the production of off-target binding which can result in reduced efficacy or toxicity. Non-limiting examples of agents suitable for formulation with the conjugates include: P-glycoprotein inhibitors (such as Pluronic P85), which can enhance entry of active agents into the CNS; biodegradable polymers, such as poly (DL-lactide-coglycolide) microspheres for sustained release delivery after intracerebral implantation; and loaded nanoparticles, such as those made of polybutylcyanoacrylate, which can deliver active agents across the blood brain barrier and can alter neuronal uptake mechanisms.

Also included herein are pharmaceutical compositions prepared for storage or administration, which include a pharmaceutically effective amount of the desired conjugates in a pharmaceutically acceptable carrier or diluent. Acceptable carriers, diluents, and/or excipients for therapeutic use are well known in the pharmaceutical art. In some embodiments, buffers, preservatives, bulking agents, dispersants, stabilizers, dyes, can be provided. In addition, antioxidants and suspending agents can be used Examples of suitable carriers, diluents and/or excipients include, but are not limited to: (1) Dulbecco's phosphate buffered saline, pH about 6.5, which would contain about 1 mg/ml to 25 mg/ml human serum albumin, (2) 0.9% saline (0.9% w/v NaCl), and (3) 5% (w/v) dextrose.

The term “pharmaceutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Pharmaceutically effective amounts for a given situation can be determined by routine experimentation that is within the skill and judgment of the clinician. In a preferred aspect, the disease or condition to can be treated via gene silencing.

For any conjugate, the pharmaceutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

In some embodiments, a drug or its derivatives, drug-conjugates or PBRM-drug conjugates can be evaluated for their ability to inhibit tumor growth in several cell lines using Cell titer Glo. Dose response curves can be generated using SoftMax Pro software and IC₅₀ values can be determined from four-parameter curve fitting. Cell lines employed can include those which are the targets of the PBRM and a control cell line that is not the target of the PBRM contained in the test conjugates.

In one embodiment, the conjugates are formulated for parenteral administration by injection including using conventional catheterization techniques or infusion. Formulations for injection may be presented in unit dosage form, e.g., in ampules or in multi-dose containers, with an added preservative. The conjugates can be administered parenterally in a sterile medium. The conjugate, depending on the vehicle and concentration used, can either be suspended or dissolved in the vehicle. Advantageously, adjuvants such as local anesthetics, preservatives, and buffering agents can be dissolved in the vehicle. The term “parenteral” as used herein includes percutaneous, subcutaneous, intravascular (e.g., intravenous), intramuscular, or intrathecal injection or infusion techniques and the like. In addition, there is provided a pharmaceutical formulation comprising conjugates and a pharmaceutically acceptable carrier. One or more of the conjugates can be present in association with one or more non-toxic pharmaceutically acceptable carriers and/or diluents and/or adjuvants, and if desired other active ingredients.

The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose, a bland fixed oil can be employed including synthetic mono- or diglycerides. In addition, fatty acids such as oleic acid find use in the preparation of injectables.

The conjugates and compositions described herein may be administered in appropriate form, preferably parenterally, more preferably intravenously. For parenteral administration, the conjugates or compositions can be aqueous or nonaqueous sterile solutions, suspensions or emulsions. Propylene glycol, vegetable oils and injectable organic esters, such as ethyl oleate, can be used as the solvent or vehicle. The compositions can also contain adjuvants, emulsifiers or dispersants.

Dosage levels of the order of from between about 0.001 mg and about 140 mg per kilogram of body weight per day are useful in the treatment of the above-indicated conditions (between about 0.05 mg and about 7 g per subject per day). In some embodiments, the dosage administered to a patient is between about 0.001 mg/kg to about 100 mg/kg of the subject's body weight. In some embodiments, the dosage administered to a patient is between about 0.01 mg/kg to about 15 mg/kg of the subject's body weight. In some embodiments, the dosage administered to a patient is between about 0.1 mg/kg and about 15 mg/kg of the subject's body weight. In some embodiments, the dosage administered to a patient is between about 0.1 mg/kg and about 20 mg/kg of the subject's body weight. In some embodiments, the dosage administered is between about 0.1 mg/kg to about 5 mg/kg or about 0.1 mg/kg to about 10 mg/kg of the subject's body weight. In some embodiments, the dosage administered is between about 1 mg/kg to about 15 mg/kg of the subject's body weight. In some embodiments, the dosage administered is between about 1 mg/kg to about 10 mg/kg of the subject's body weight. The amount of conjugate that can be combined with the carrier materials to produce a single dosage form varies depending upon the host treated and the particular mode of administration. Dosage unit forms can generally contain from between about 0.001 mg and about 100 mg; between about 0.01 mg and about 75 mg; or between about 0.01 mg and about 50 mg; or between about 0.01 mg and about 25 mg; of a conjugate.

For intravenous administration, the dosage levels can comprise ranges described in the preceding paragraphs, or from about 0.01 to about 200 mg of a conjugate per kg of the animal's body weight. In one aspect, the composition can include from about 1 to about 100 mg of a conjugate per kg of the animal's body weight. In another aspect, the amount administered will be in the range from about 0.1 to about 25 mg/kg of body weight of a compound.

In some embodiments, the conjugates can be administered are as follows. The conjugates can be given daily for about 5 days either as an i.v., bolus each day for about 5 days, or as a continuous infusion for about 5 days.

Alternatively, the conjugates can be administered once a week for six weeks or longer. As another alternative, the conjugates can be administered once every two or three weeks. Bolus doses are given in about 50 to about 400 ml of normal saline to which about 5 to about 10 ml of human serum albumin can be added. Continuous infusions are given in about 250 to about 500 ml of normal saline, to which about 25 to about 50 ml of human serum albumin can be added, per 24 hour period.

In some embodiments, about one to about four weeks after treatment, the patient can receive a second course of treatment. Specific clinical protocols with regard to route of administration, excipients, diluents, dosages, and times can be determined by the skilled artisan as the clinical situation warrants.

In other embodiments, the therapeutically effective amount may be provided on another regular schedule, i.e., daily, weekly, monthly, or yearly basis or on an irregular schedule with varying administration days, weeks, months, etc. Alternatively, the therapeutically effective amount to be administered may vary. In one embodiment, the therapeutically effective amount for the first dose is higher than the therapeutically effective amount for one or more of the subsequent doses. In another embodiment, the therapeutically effective amount for the first dose is lower than the therapeutically effective amount for one or more of the subsequent doses. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the recommendations of the relevant regulatory bodies and judgment of a health-care practitioner. The therapeutically effective amounts described herein refer to total amounts administered for a given time period; that is, if more than one different conjugate described herein is administered, the therapeutically effective amounts correspond to the total amount administered. It is understood that the specific dose level for a particular subject depends upon a variety of factors including the activity of the specific conjugate, the age, body weight, general health, sex, diet, time of administration, route of administration, and rate of excretion, combination with other active agents, and the severity of the particular disease undergoing therapy.

In some embodiments, a therapeutically effective amount of a conjugate disclosed herein relates generally to the amount needed to achieve a therapeutic objective. As noted above, this may be a binding interaction between the antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. Common ranges for therapeutically effective dosing of conjugates disclosed herein may be, by way of nonlimiting example, from about 0.1 mg/kg body weight to about 50 mg/kg body weight, from about 0.1 mg/kg body weight to about 100 mg/kg body weight or from about 0.1 mg/kg body weight to about 150 mg/kg body weight. Common dosing frequencies may range, for example, from twice daily to once a month (e.g., once daily, once weekly; once every other week; once every 3 weeks or monthly). For example, conjugates disclosed herein can be administered (e.g., as a single dose weekly, every 2 weeks, every 3 weeks, or monthly) at about 0.1 mg/kg to about 20 mg/kg (e.g., 0.2 mg/kg, 0.5 mg/kg, 0.67 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg). For example, conjugates disclosed herein can be administered (e.g., as a single dose weekly, every 2 weeks, every 3 weeks, or monthly) at about 0.1 mg/kg to about 20 mg/kg (e.g., 0.2 mg/kg, 0.5 mg/kg, 0.67 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 19 mg/kg, or 20 mg/kg) for treating cancer.

For administration to non-human animals, the conjugates can also be added to the animal feed or drinking water. It can be convenient to formulate the animal feed and drinking water so that the animal takes in a therapeutically appropriate quantity of the conjugates along with its diet. It can also be convenient to present the conjugates as a premix for addition to the feed or drinking water.

The conjugates can also be administered to a subject in combination with other therapeutic compounds to increase the overall therapeutic effect. The use of multiple compounds to treat an indication can increase the beneficial effects while reducing the presence of side effects. In some embodiments, the conjugates are used in combination with chemotherapeutic agents, such as those disclosed in U.S. Pat. No. 7,303,749. In other embodiments the chemotherapeutic agents, include, but are not limited to letrozole, oxaliplatin, docetaxel, 5-FU, lapatinib, capecitabine, leucovorin, erlotinib, pertuzumab, bevacizumab, and gemcitabine.

The present disclosure also provides pharmaceutical kits comprising one or more containers filled with one or more of the conjugates and/or compositions of the present disclosure, including, one or more chemotherapeutic agents. Such kits can also include, for example, other compounds and/or compositions, a device(s) for administering the compounds and/or compositions, and written instructions in a form prescribed by a governmental agency regulating the manufacture, use or sale of pharmaceuticals or biological products. The compositions described herein can be packaged as a single dose or for continuous or periodic discontinuous administration. For continuous administration, a package or kit can include the conjugates in each dosage unit (e.g., solution or other unit described above or utilized in drug delivery), and optionally instructions for administering the doses daily, weekly, or monthly, for a predetermined length of time or as prescribed. If varying concentrations of a composition, of the components of the composition, or the relative ratios of the conjugates or agents within a composition over time is desired, a package or kit may contain a sequence of dosage units which provide the desired variability.

A number of packages or kits are known in the art for dispensing pharmaceutical agents for periodic oral use. In one embodiment, the package has indicators for each period. In another embodiment, the package is a labeled blister package, dial dispenser package, or bottle. The packaging means of a kit may itself be geared for administration, such as a syringe, pipette, eye dropper, or other such apparatus, from which the formulation may be applied to an affected area of the body, injected into a subject, or even applied to and mixed with the other components of the kit.

### Methods of Use

In some aspects, the present disclosure provides a method of treating a subject in need thereof (preferably mammals, most preferably humans and includes males, females, infants, children and adults) by administering a pharmaceutically effective amount of the conjugate (e.g., the antibody-drug conjugate (ADC)) of the present disclosure. In some embodiments, the conjugate (e.g., the antibody-drug conjugate (ADC)) of the present disclosure is administered in the form of soluble linear polymers, copolymers, conjugates, colloids, particles, gels, solid items, fibers, films, etc. Biodegradable biocompatible conjugates of the present disclosure can be used as drug carriers and drug carrier components, in systems of controlled drug release, preparations for low-invasive surgical procedures, etc. Pharmaceutical formulations can be injectable, implantable, etc.

In some aspects, the present disclosure provides a method of treating or preventing a disease or disorder in a subject in need thereof, comprising administering to the subject a pharmaceutically effective amount of a conjugate (e.g., an antibody-drug conjugate (ADC)) of the present disclosure; wherein said conjugate releases one or more PBD drug moieties upon biodegradation.

In some embodiments, the disease or disorder to be treated is a hyperproliferative disease, e.g., cancer.

In some embodiments, the conjugate (e.g., the antibody-drug conjugate (ADC)) of the present disclosure can be administered in vitro, in vivo and/or ex vivo to treat patients and/or to modulate the growth of selected cell populations including, for example, cancer.

In some aspects, the present disclosure provides a method of treating cancer, comprising administering to the subject a pharmaceutically effective amount of a conjugate (e.g., an antibody-drug conjugate (ADC)) of the present disclosure. In some embodiments, the particular types of cancers that can be treated with the conjugates of the present disclosure include, but are not limited to: (1) solid tumors, including but not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophogeal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma, multiforme astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma; (2) blood-borne cancers, including but not limited to acute lymphoblastic leukemia “ALL”, acute lymphoblastic B-cell leukemia, acute lymphoblastic T-cell leukemia, acute myeloblastic leukemia “AML”, acute promyelocytic leukemia “APL”, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia “CML”, chronic lymphocytic leukemia “CLL”, hairy cell leukemia, multiple myeloma, acute and chronic leukemias, e.g., lymphoblastic myelogenous and lymphocytic myelocytic leukemias; and (3) lymphomas such as Hodgkin's disease, non-Hodgkin's Lymphoma, Multiple myeloma, Waldenstrom's macroglobulinemia, Heavy chain disease, and Polycythemia vera.

In some embodiments, the conjugate (e.g., the antibody-drug conjugate (ADC)) of the present disclosure can be administered in vitro, in vivo and/or ex vivo to treat autoimmune diseases.

In some aspects, the present disclosure provides a method of treating an autoimmune disease, comprising administering to the subject a pharmaceutically effective amount of a conjugate (e.g., an antibody-drug conjugate (ADC)) of the present disclosure. In some embodiments, the autoimmune diseases that can be treated with the conjugates of the present disclosure include, but are not limited to, systemic lupus, rheumatoid arthritis, psoriasis, and multiple sclerosis; graft rejections, such as renal transplant rejection, liver transplant rejection, lung transplant rejection, cardiac transplant rejection, and bone marrow transplant rejection; graft versus host disease; viral infections, such as CMV infection, HIV infection, and AIDS; and parasite infections, such as giardiasis, amoebiasis, schistosomiasis, and the like.

In some aspects, the present disclosure provides a conjugate disclosed herein for use in the manufacture of a medicament useful for treating or lessening the severity of disorders, such as, characterized by abnormal growth of cells (e.g., cancer).

In some embodiments, the PBD drug moiety is locally delivered to a specific target cell, tissue, or organ.

In some aspects, the present disclosure provides a method of treating a disease or disorder in a subject, comprising preparing an aqueous formulation of at least one conjugate of the present disclosure and parenterally injecting said formulation in the subject.

In some aspects, the present disclosure provides a method of treating a disease or disorder in a subject, comprising preparing an implant comprising at least one conjugate of the present disclosure, and implanting said implant into the subject. In some embodiments, the implant is a biodegradable gel matrix.

In some aspects, the present disclosure provides a method for treating of a subject in need thereof, comprising administering a conjugate according to the methods described above.

In some aspects, the present disclosure provides a method for eliciting an immune response in a subject, comprising administering a conjugate as in the methods described above.

In some aspects, the present disclosure provides a method of diagnosing a disease in a subject, comprising steps of:

administering a conjugate of the present disclosure, wherein the conjugate further comprises a detectable molecule; and

detecting the detectable molecule.

In some embodiments, the step of detecting the detectable molecule is performed non-invasively. In some embodiments, the step of detecting the detectable molecule is performed using suitable imaging equipment.

In some embodiments, the present disclosure provides a method for treating an animal comprises administering to the animal a biodegradable biocompatible conjugate of the present disclosure as a packing for a surgical wound from which a tumor or growth has been removed. The biodegradable biocompatible conjugate packing will replace the tumor site during recovery and degrade and dissipate as the wound heals.

In some embodiments, soluble or colloidal conjugates of the present disclosure are administered intravenously. In some embodiments, soluble or colloidal conjugates of the present disclosure are administered via local (e.g., subcutaneous, intramuscular) injection. In some embodiments, solid conjugates of the present disclosure (e.g., particles, implants, drug delivery systems) are administered via implantation or injection.

In some embodiments, conjugates of the present disclosure comprising a detectable label are administered to study the patterns and dynamics of label distribution in animal body.

In some embodiments, the conjugate is associated with a diagnostic label for in vivo monitoring.

The conjugates described above can be used for therapeutic, preventative, and analytical (diagnostic) treatment of animals. The conjugates are intended, generally, for parenteral administration, but in some cases may be administered by other routes.

In some embodiments, soluble or colloidal conjugates are administered intravenously. In another embodiment, soluble or colloidal conjugates are administered via local (e.g., subcutaneous, intramuscular) injection. In another embodiment, solid conjugates (e.g., particles, implants, drug delivery systems) are administered via implantation or injection.

In another embodiment, conjugates comprising a detectable label are administered to study the patterns and dynamics of label distribution in animal body.

In some embodiments, any one or more of the conjugates disclosed herein may be used in practicing any of the methods described herein.

### Diagnostic and Prophylactic Formulations

The PBD antibody conjugates disclosed herein are used in diagnostic and prophylactic formulations. In one embodiment, a PBD antibody conjugate disclosed herein is administered to patients that are at risk of developing one or more of the aforementioned diseases, such as for example, without limitation, cancer. A patient's or organ's predisposition to one or more of the aforementioned indications can be determined using genotypic, serological or biochemical markers.

In another embodiment of the disclosure, a PBD antibody conjugate disclosed herein is administered to human individuals diagnosed with a clinical indication associated with one or more of the aforementioned diseases, such as for example, without limitation, cancer. Upon diagnosis, a PBD antibody conjugate disclosed herein is administered to mitigate or reverse the effects of the clinical indication associated with one or more of the aforementioned diseases. All methods described herein can be performed in any suitable order unless otherwise indicated herein or otherwise clearly contradicted by context. The use of any and all examples, or exemplary language (e.g., “such as”) provided herein, is intended merely to better illustrate the invention and is not to be construed as a limitation on the scope of the claims unless explicitly otherwise claimed. No language in the specification is to be construed as indicating that any non-claimed element is essential to what is claimed.

### Definitions

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. In some embodiments, C₁-C₆ alkyl is intended to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, s-pentyl or n-hexyl.

In certain embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “cycloalkyl” refers to a saturated or unsaturated nonaromatic hydrocarbon mono- or multi-ring (e.g., fused, bridged, or spiro rings) system having 3 to 30 carbon atoms (e.g., C₃-C₁₀). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. The term “heterocycloalkyl” refers to a saturated or unsaturated nonaromatic ring system having one or more heteroatoms (such as O, N, S, P, or Se) as ring atoms, such as a 3-8 membered monocyclic, 7-12 membered bicyclic (fused, bridged, or spiro rings), or 11-14 membered tricyclic ring system (fused, bridged, or spiro rings) having, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, and the like. In the case of multicyclic non-aromatic rings, only one of the rings needs to be non-aromatic (e.g., 1,2,3,4-tetrahydronaphthalenyl or 2,3-dihydroindole). The terms “cycloalkylene” and “heterocycloalkylene” refer to the corresponding divalent groups, respectively.

The term “optionally substituted alkyl” refers to unsubstituted alkyl or alkyl having designated substituents replacing one or more hydrogen atoms on one or more carbons of the hydrocarbon backbone. Such substituents can include, In some embodiments, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, “alkyl linker” or “alkylene linker” is intended to include C₁, C₂, C₃, C₄, C₅ or C₆ straight chain (linear or branched) saturated divalent aliphatic hydrocarbon groups and C₃, C₄, C₅ or C₆ branched saturated aliphatic hydrocarbon groups. In some embodiments, C₁-C₆ alkylene linker is intended to include C₁, C₂, C₃, C₄, C₅ and C₆ alkylene linker groups. Examples of alkylene linker include, moieties having from one to six carbon atoms, such as, but not limited to, methyl (—CH₂—), ethyl (—CH₂CH₂—), n-propyl (—CH₂CH₂CH₂—), i-propyl (—CHCH₃CH₂—), n-butyl (—CH₂CH₂CH₂CH₂—), s-butyl (—CHCH₃CH₂CH₂—), i-butyl (—C(CH₃)₂CH₂—), n-pentyl (—CH₂CH₂CH₂CH₂CH₂—), s-pentyl (—CHCH₃CH₂CH₂CH₂—) or n-hexyl (—CH₂CH₂CH₂CH₂CH₂CH₂—).

“Alkenyl” includes unsaturated aliphatic groups analogous in length and possible substitution to the alkyls described above, but that contain at least one double bond. In some embodiments, the term “alkenyl” includes straight chain alkenyl groups (e.g., ethenyl, propenyl, butenyl, pentenyl, hexenyl, heptenyl, octenyl, nonenyl, decenyl), and branched alkenyl groups.

In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

The term “optionally substituted alkenyl” refers to unsubstituted alkenyl or alkenyl having designated substituents replacing one or more hydrogen atoms on one or more hydrocarbon backbone carbon atoms. Such substituents can include, In some embodiments, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

“Alkynyl” includes unsaturated aliphatic groups analogous in length and possible substitution to the alkyls described above, but which contain at least one triple bond. In some embodiments, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms.

The term “optionally substituted alkynyl” refers to unsubstituted alkynyl or alkynyl having designated substituents replacing one or more hydrogen atoms on one or more hydrocarbon backbone carbon atoms. Such substituents can include, In some embodiments, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. In some embodiments, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

“Aryl” includes groups with aromaticity, including “conjugated,” or multicyclic systems with one or more aromatic rings and do not contain any heteroatom in the ring structure. Examples include phenyl, naphthalenyl, etc. The term “arylene” refers to the corresponding divalent groups, such as phenylene.

“Heteroaryl” groups are aryl groups, as defined above, except having from one to four heteroatoms in the ring structure, and may also be referred to as “aryl heterocycles” or “heteroaromatics.” As used herein, the term “heteroaryl” is intended to include a stable aromatic heterocyclic ring, such as a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidized (i.e., N→O and S(O)ₚ, where p=1 or 2). It is to be noted that total number of S and O atoms in the aromatic heterocycle is not more than 1.

Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like. The term “heteroarylene” refers to the corresponding divalent groups.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthyridine, indole, benzofuran, purine, benzofuran, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, In some embodiments, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, “carbocycle” or “carbocyclic ring” is intended to include any stable monocyclic, bicyclic or tricyclic ring having the specified number of carbons, any of which may be saturated, unsaturated, or aromatic. Carbocycle includes cycloalkyl and aryl. In some embodiments, a C₃-C₁₄ carbocycle is intended to include a monocyclic, bicyclic or tricyclic ring having 3, 4, 5, 6, 7, 8, 9, 10, 11, 12, 13 or 14 carbon atoms. Examples of carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, fluorenyl, phenyl, naphthyl, indanyl, adamantyl and tetrahydronaphthyl. Bridged rings are also included in the definition of carbocycle, including, In some embodiments, [3.3.0]bicyclooctane, [4.3.0]bicyclononane, and [4.4.0] bicyclodecane and

bicyclooctane. A bridged ring occurs when one or more carbon atoms link two non-adjacent carbon atoms. In some embodiments, bridge rings are one or two carbon atoms. It is noted that a bridge always converts a monocyclic ring into a tricyclic ring. When a ring is bridged, the substituents recited for the ring may also be present on the bridge. Fused (e.g., naphthyl, tetrahydronaphthyl) and spiro rings are also included.

As used herein, “heterocycle” or “heterocyclic group” includes any ring structure (saturated, unsaturated, or aromatic) which contains at least one ring heteroatom (e.g., 1-4 heteroatoms selected from N, O and S). Heterocycle includes heterocycloalkyl and heteroaryl. Examples of heterocycles include, but are not limited to, morpholine, pyrrolidine, tetrahydrothiophene, piperidine, piperazine, oxetane, pyran, tetrahydropyran, azetidine, and tetrahydrofuran.

Examples of heterocyclic groups include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H, 6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl (e.g., benzo[d][1,3]dioxole-5-yl), morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, 1,2,4-oxadiazol5(4H)-one, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl and xanthenyl.

The term “substituted,” as used herein, means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, In some embodiments, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

The term “hydroxy” or “hydroxyl” includes groups with an —OH or —O.

As used herein, “halo” or “halogen” refers to fluoro, chloro, bromo and iodo. The term “perhalogenated” generally refers to a moiety wherein all hydrogen atoms are replaced by halogen atoms. The term “haloalkyl” or “haloalkoxyl” refers to an alkyl or alkoxyl substituted with one or more halogen atoms.

As used herein, the term “bis-oxy-alkylene” refers —O-alkylene-O—, in which alkylene can be linear or branched, e.g., —CH₂—, —CH(CH₃)₂—, or —(CH₂)₂—.

The term “carbonyl” includes compounds and moieties which contain a carbon connected with a double bond to an oxygen atom. Examples of moieties containing a carbonyl include, but are not limited to, aldehydes, ketones, carboxylic acids, amides, esters, anhydrides, etc.

The term “carboxyl” refers to —COOH or its C₁-C₆ alkyl ester.

“Acyl” includes moieties that contain the acyl radical (R—C(O)—) or a carbonyl group. “Substituted acyl” includes acyl groups where one or more of the hydrogen atoms are replaced by, In some embodiments, alkyl groups, alkynyl groups, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Aroyl” includes moieties with an aryl or heteroaromatic moiety bound to a carbonyl group. Examples of aroyl groups include phenylcarboxy, naphthyl carboxy, etc.

“Alkoxyalkyl,” “alkylaminoalkyl,” and “thioalkoxyalkyl” include alkyl groups, as described above, wherein oxygen, nitrogen, or sulfur atoms replace one or more hydrocarbon backbone carbon atoms.

The term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

The term “ether” or “alkoxy” includes compounds or moieties which contain an oxygen bonded to two carbon atoms or heteroatoms. In some embodiments, the term includes “alkoxyalkyl,” which refers to an alkyl, alkenyl, or alkynyl group covalently bonded to an oxygen atom which is covalently bonded to an alkyl group.

The term “ester” includes compounds or moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc.

The term “thioalkyl” includes compounds or moieties which contain an alkyl group connected with a sulfur atom. The thioalkyl groups can be substituted with groups such as alkyl, alkenyl, alkynyl, halogen, hydroxyl, alkylcarbonyloxy, arylcarbonyloxy, alkoxycarbonyloxy, aryloxycarbonyloxy, carboxylate, carboxyacid, alkylcarbonyl, arylcarbonyl, alkoxycarbonyl, aminocarbonyl, alkylaminocarbonyl, dialkylaminocarbonyl, alkylthiocarbonyl, alkoxyl, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties.

The term “thiocarbonyl” or “thiocarboxy” includes compounds and moieties which contain a carbon connected with a double bond to a sulfur atom.

The term “thioether” includes moieties which contain a sulfur atom bonded to two carbon atoms or heteroatoms. Examples of thioethers include, but are not limited to alkthioalkyls, alkthioalkenyls, and alkthioalkynyls. The term “alkthioalkyls” include moieties with an alkyl, alkenyl, or alkynyl group bonded to a sulfur atom which is bonded to an alkyl group. Similarly, the term “alkthioalkenyls” refers to moieties wherein an alkyl, alkenyl or alkynyl group is bonded to a sulfur atom which is covalently bonded to an alkenyl group; and alkthioalkynyls” refers to moieties wherein an alkyl, alkenyl or alkynyl group is bonded to a sulfur atom which is covalently bonded to an alkynyl group.

As used herein, “amine” or “amino” refers to —NH₂. “Alkylamino” includes groups of compounds wherein the nitrogen of —NH₂ is bound to at least one alkyl group. Examples of alkylamino groups include benzylamino, methylamino, ethylamino, phenethylamino, etc. “Dialkylamino” includes groups wherein the nitrogen of —NH₂ is bound to two alkyl groups. Examples of dialkylamino groups include, but are not limited to, dimethylamino and diethylamino. “Arylamino” and “diarylamino” include groups wherein the nitrogen is bound to at least one or two aryl groups, respectively. “Aminoaryl” and “aminoaryloxy” refer to aryl and aryloxy substituted with amino. “Alkylarylamino,” “alkylaminoaryl” or “arylaminoalkyl” refers to an amino group which is bound to at least one alkyl group and at least one aryl group. “Alkaminoalkyl” refers to an alkyl, alkenyl, or alkynyl group bound to a nitrogen atom which is also bound to an alkyl group. “Acylamino” includes groups wherein nitrogen is bound to an acyl group. Examples of acylamino include, but are not limited to, alkylcarbonylamino, arylcarbonylamino, carbamoyl and ureido groups.

The term “amide” or “aminocarboxy” includes compounds or moieties that contain a nitrogen atom that is bound to the carbon of a carbonyl or a thiocarbonyl group. The term includes “alkaminocarboxy” groups that include alkyl, alkenyl or alkynyl groups bound to an amino group which is bound to the carbon of a carbonyl or thiocarbonyl group. It also includes “arylaminocarboxy” groups that include aryl or heteroaryl moieties bound to an amino group that is bound to the carbon of a carbonyl or thiocarbonyl group. The terms “alkylaminocarboxy”, “alkenylaminocarboxy”, “alkynylaminocarboxy” and “arylaminocarboxy” include moieties wherein alkyl, alkenyl, alkynyl and aryl moieties, respectively, are bound to a nitrogen atom which is in turn bound to the carbon of a carbonyl group. Amides can be substituted with substituents such as straight chain alkyl, branched alkyl, cycloalkyl, aryl, heteroaryl or heterocycle. Substituents on amide groups may be further substituted.

Compounds of the present disclosure that contain nitrogens can be converted to N-oxides by treatment with an oxidizing agent (e.g., 3-choroperoxybenzoic acid (m-CPBA) and/or hydrogen peroxides) to afford other compounds of the present disclosure. Thus, all shown and claimed nitrogen-containing compounds are considered, when allowed by valency and structure, to include both the compound as shown and its N-oxide derivative (which can be designated as N→O or N⁺—O⁻). Furthermore, in other instances, the nitrogens in the compounds of the present disclosure can be converted to N-hydroxy or N-alkoxy compounds. In some embodiments, N-hydroxy compounds can be prepared by oxidation of the parent amine by an oxidizing agent such as m-CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

In the present specification, the structural formula of the compound represents a certain isomer for convenience in some cases, but the present disclosure includes all isomers, such as geometrical isomers, optical isomers based on an asymmetrical carbon, stereoisomers, tautomers, and the like, it being understood that not all isomers may have the same level of activity. In addition, a crystal polymorphism may be present for the compounds represented by the formula. It is noted that any crystal form, crystal form mixture, or anhydride or hydrate thereof is included in the scope of the present disclosure.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

A carbon atom bonded to four nonidentical substituents is termed a “chiral center.”

“Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds or a cycloalkyl linker (e.g., 1,3-cylcobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It should also be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

Furthermore, the structures and other compounds discussed in this disclosure include all atropic isomers thereof, it being understood that not all atropic isomers may have the same level of activity. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

The compounds of any Formula described herein include the compounds themselves, as well as their salts, and their solvates, if applicable. A salt, In some embodiments, can be formed between an anion and a positively charged group (e.g., amino) on a compound of the disclosure. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). The term “pharmaceutically acceptable anion” refers to an anion suitable for forming a pharmaceutically acceptable salt. Likewise, a salt can also be formed between a cation and a negatively charged group (e.g., carboxylate) on a compound of the disclosure. Suitable cations include sodium ion, potassium ion, magnesium ion, calcium ion, and an ammonium cation such as tetramethylammonium ion. Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. The compounds of the disclosure also include those salts containing quaternary nitrogen atoms.

Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Additionally, the compounds of the present disclosure, In some embodiments, the salts of the compounds, can exist in either hydrated or unhydrated (the anhydrous) form or as solvates with other solvent molecules. Non-limiting examples of hydrates include monohydrates, dihydrates, etc. Non-limiting examples of solvates include ethanol solvates, acetone solvates, etc.

“Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O. A hydrate refers to, In some embodiments, a mono-hydrate, a di-hydrate, a tri-hydrate, etc.

It may be convenient or desirable to prepare, purify, and/or handle a corresponding solvate of an active compound. Compounds of the disclosure include compounds where a nucleophilic solvent (H₂O, R^{A}OH, R^{A}NH₂, R^{A}SH) adds across the imine bond of the PBD moiety, which is illustrated below where the solvent is water or an alcohol (R^{A}OH, where R^{A} is an ether substituent as described above):

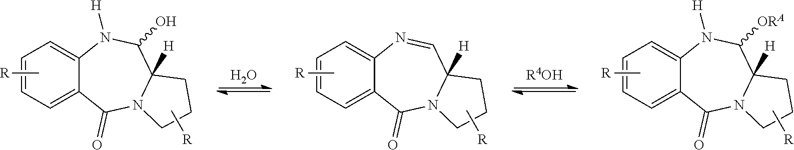

These forms can be called the carbinolamine and carbinolamine ether forms of the PBD. The balance of these equilibria depend on the conditions in which the compounds are found, as well as the nature of the moiety itself.

These compounds may be isolated in solid form, In some embodiments, by lyophilisation.

As defined herein, the term “derivative” refers to compounds that have a common core structure, and are substituted with various groups as described herein. In some embodiments, all of the compounds represented by Formula (I) are pyrrolo[2,1-c][1, 4]benzodiazepines compounds (PBDs), and have Formula (I) as a common core.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonimides, tetrazoles, sulfonates and phosphonates. See, e.g., Patani and LaVoie, Chem. Rev. 96, 3147-3176, 1996.

The present disclosure is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

The present disclosure provides methods for the synthesis of the compounds of any of the Formulae and conjugates thereof described herein. The present disclosure also provides detailed methods for the synthesis of various disclosed conjugates of the present disclosure according to the following schemes as shown in the Examples.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

The synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

Compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Afechanisms, and Structure, 5^{th} edition, John Wiley & Sons: New York, 2001; Greene, T. W., Wuts, P. G. M., Protective Groups in Organic Synthesis, 4^{th} Edition, Wiley-Interscience, 2007; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art. The following descriptions of synthetic methods are designed to illustrate, but not to limit, general procedures for the preparation of compounds of the present disclosure.

“Protein based recognition-molecule” or “PBRM” refers to a molecule that recognizes and binds to a cell surface marker or receptor such as, a transmembrane protein, surface immobilized protein, or proteoglycan. Examples of PBRMs include but are not limited to, antibodies (e.g., Trastuzumab, Cetuximab, Rituximab, Bevacizumab, Epratuzumab, Veltuzumab, Labetuzumab, B7-H₄, B7-H₃, CA125, CD33, CXCR2, EGFR, FGFR1, FGFR2, FGFR3, FGFR4, HER2, NaPi2b, c-Met, NOTCH1, NOTCH2, NOTCH3, NOTCH4, PD-L1, c-Kit, MUC1, MUC13 and anti-5T4) or peptides (LHRH receptor targeting peptides, EC-1 peptide), lipocalins, such as, In some embodiments, anticalins, proteins such as, In some embodiments, interferons, lymphokines, growth factors, colony stimulating factors, and the like, peptides or peptide mimics, and the like. The protein based recognition molecule, in addition to targeting the conjugate to a specific cell, tissue or location, may also have certain therapeutic effect such as antiproliferative (cytostatic and/or cytotoxic) activity against a target cell or pathway. The protein based recognition molecule comprises or may be engineered to comprise at least one chemically reactive group such as, —COOH, primary amine, secondary amine —NHR, —SH, or a chemically reactive amino acid moiety or side chains such as, In some embodiments, tyrosine, histidine, cysteine, or lysine. In some embodiments, a PBRM may be a ligand (LG) or targeting moiety which specifically binds or complexes with a cell surface molecule, such as a cell surface receptor or antigen, for a given target cell population. Following specific binding or complexing of the ligand with its receptor, the cell is permissive for uptake of the ligand or ligand-drug-conjugate, which is then internalized into the cell. As used herein, a ligand that “specifically binds or complexes with” or “targets” a cell surface molecule preferentially associates with a cell surface molecule via intermolecular forces. In some embodiments, the ligand can preferentially associate with the cell surface molecule with a K_{d} of less than about 50 nM, less than about 5 nM, or less than 500 pM. Techniques for measuring binding affinity of a ligand to a cell surface molecule are well-known; In some embodiments, one suitable technique, is termed surface plasmon resonance (SPR). In some embodiments, the ligand is used for targeting and has no detectable therapeutic effect as separate from the drug which it delivers. In another embodiment, the ligand functions both as a targeting moiety and as a therapeutic or immunomodulatory agent (e.g., to enhance the activity of the active drug or prodrug).

### Synthetic Methods

The conjugates of this disclosure having any of the Formulae described herein may be prepared according to the procedures illustrated in Scheme 1 and the Examples, from commercially available starting materials or starting materials which can be prepared using literature procedures.

Any available techniques can be used to make the conjugates or compositions thereof, and intermediates and components (e.g., scaffolds) useful for making them. For example, semi-synthetic and fully synthetic methods may be used.

The general methods of producing the conjugates or scaffolds disclosed herein are illustrated in Scheme 1 below. More specific methods of syntheses of the conjugates are described in the Examples and for the scaffolds in co-pending application U.S. 62/572,010 filed Oct. 13, 2017. The variables (e.g., M^{P}, M^{A}, W^{D}, L^{D}, and L^{P′}, etc.) in these schemes have the same definitions as described herein unless otherwise specified.

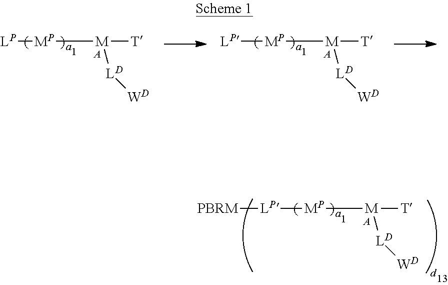

The synthetic processes of the disclosure can tolerate a wide variety of functional groups; therefore various substituted starting materials can be used. The processes generally provide the desired final compound at or near the end of the overall process, although it may be desirable in certain instances to further convert the compound to a pharmaceutically acceptable salt, ester or prodrug thereof.

PBD compounds used for the conjugates of the present disclosure can be prepared in a variety of ways using commercially available starting materials, compounds known in the literature, as described in co-pending application U.S. Ser. No. 15/597,453 filed May 17, 2017, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; and Greene, T. W., Wuts, P. G. M., Protective Groups in Organic Synthesis, 3^{rd} edition, John Wiley & Sons: New York, 1999, incorporated by reference herein, are useful and recognized reference textbooks of organic synthesis known to those in the art. The following descriptions of synthetic methods are designed to illustrate, but not to limit, general procedures for the preparation of compounds of the present disclosure.

Conjugates of the present disclosure can be conveniently prepared by a variety of methods familiar to those skilled in the art. The conjugates of the disclosure with each of the formulae described herein may be prepared according to the following procedures from commercially available starting materials or starting materials which can be prepared using literature procedures. These procedures show the preparation of representative conjugates of this disclosure.

Conjugates designed, selected and/or optimized by methods described above, once produced, can be characterized using a variety of assays known to those skilled in the art to determine whether the conjugates have biological activity. In some embodiments, the conjugates can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the conjugate molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below. Conjugates of the present disclosure can also be prepared in a variety of ways using commercially available starting materials, compounds, antibodies, and antibody fragments each of which are known in the literature, or from readily prepared intermediates, by employing standard synthetic methods and procedures either known to those skilled in the art, or which will be apparent to the skilled artisan in light of the teachings herein. In some embodiments, for the synthesis of conjugates of compounds of Formula (IV), where the antibody or antibody fragment is directly or indirectly linked to the compound at position E″ or D″, methods and linkers disclosed in W02011/13063, WO2011/130616, WO2015/159076, WO2015/052535, WO2015/052534, WO2015/052321, WO2014/130879, WO2014/096365, WO2014/057122, WO2014/057073, WO2013/164593, WO2013/055993, WO2013/055990, WO2013/053873, WO2013/053871, WO2013/041606, WO2011/130616, and WO2011/130613 may be used. Each of these publications is incorporated herein by reference in its entirety.

As another example, for the synthesis of conjugates of compounds of Formula (IV), where the antibody or antibody fragment is directly or indirectly linked to the compound at position R″₇, methods and linkers disclosed in WO2014140174(A¹) and WO2016/037644 may be used. Each of these publications is incorporated herein by reference in its entirety.

As another example, for the synthesis of conjugates of compounds of Formula (IV), where the antibody or antibody fragment is directly or indirectly linked to the compound at position R″₁₀, methods and linkers disclosed in WO 2013/055987, WO 2016/044560, WO 2016/044396, WO2015/159076, WO2015/095227, WO2015/095124, WO2015/052535, WO2015/052534, WO2015/052322, WO2014/174111, WO2014/096368, WO2014/057122, WO2014/057074, WO2014/022679, WO2014/011519, WO2014/011518, WO2013/177481, WO2013/055987, WO2011/130598, and WO2011/128650 may be used. Each of these publications is incorporated herein by reference in its entirety.

Also included are pharmaceutical compositions comprising one or more conjugates as disclosed herein in an acceptable carrier, such as a stabilizer, buffer, and the like. The conjugates can be administered and introduced into a subject by standard means, with or without stabilizers, buffers, and the like, to form a pharmaceutical composition. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal, oral or parenteral administration including intravenous, intraarterial, subcutaneous, intraperitoneal or intramuscular injection or infusion or intracranial, e.g., intrathecal or intraventricular, administration. The conjugates can be formulated and used as sterile solutions and/or suspensions for injectable administration; lyophilized powders for reconstitution prior to injection/infusion; topical compositions; as tablets, capsules, or elixirs for oral administration; or suppositories for rectal administration, and the other compositions known in the art.

A pharmacological composition or formulation refers to a composition or formulation in a form suitable for administration, e.g., systemic administration, into a cell or subject, including for example a human. Suitable forms, in part, depend upon the use or the route of entry, for example oral, inhaled, transdermal, or by injection/infusion. Such forms should not prevent the composition or formulation from reaching a target cell (i.e., a cell to which the drug is desirable for delivery). In some embodiments, pharmacological compositions injected into the blood stream should be soluble. Other factors are known in the art, and include considerations such as toxicity and forms that prevent the composition or formulation from exerting its effect.

By “systemic administration” is meant in vivo systemic absorption or accumulation of the modified polymer in the blood stream followed by distribution throughout the entire body. Administration routes that lead to systemic absorption include, without limitation: intravenous, subcutaneous, intraperitoneal, inhalation, oral, intrapulmonary, and intramuscular. Each of these administration routes exposes the compound or conjugate to an accessible diseased tissue. The rate of entry of an active agent into the circulation has been shown to be a function of molecular weight or size. The use of a conjugate (e.g., an antibody-drug conjugate (ADC)) of this disclosure can localize the drug delivery in certain cells, such as cancer cells via the specificity of antibodies.

A “pharmaceutically acceptable formulation” means a composition or formulation that allows for the effective distribution of the conjugates in the physical location most suitable for their desired activity. In some embodiments, effective delivery occurs before clearance by the reticuloendothelial system or the production of off-target binding which can result in reduced efficacy or toxicity. Non-limiting examples of agents suitable for formulation with the conjugates include: P-glycoprotein inhibitors (such as Pluronic P85), which can enhance entry of active agents into the CNS; biodegradable polymers, such as poly (DL-lactide-coglycolide) microspheres for sustained release delivery after intracerebral implantation; and loaded nanoparticles, such as those made of polybutylcyanoacrylate, which can deliver active agents across the blood brain barrier and can alter neuronal uptake mechanisms.

Also included herein are pharmaceutical compositions prepared for storage or administration, which include an effective amount of the desired conjugates in a pharmaceutically acceptable carrier or diluent. Acceptable carriers, diluents, and/or excipients for therapeutic use are well known in the pharmaceutical art. In some embodiments, buffers, preservatives, bulking agents, dispersants, stabilizers, dyes, can be provided. In addition, antioxidants and suspending agents can be used. Examples of suitable carriers, diluents and/or excipients include, but are not limited to: (1) Dulbecco's phosphate buffered saline, pH about 6.5, which would contain about 1 mg/ml to 25 mg/ml human serum albumin, (2) 0.9% saline (0.9% w/v NaCl), and (3) 5% (w/v) dextrose.

The term “effective amount”, as used herein, refers to an amount of a pharmaceutical agent to treat, ameliorate, or prevent an identified disease or condition, or to exhibit a detectable therapeutic or inhibitory effect. The effect can be detected by any assay or method known in the art. The precise effective amount for a subject will depend upon the subject's body weight, size, and health; the nature and extent of the condition; and the therapeutic or combination of therapeutics selected for administration. Effective amounts for a given situation can be determined by routine experimentation that is within the skill and judgment of the clinician. In a preferred aspect, the disease or condition to can be treated via gene silencing.

For any conjugate, the effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, L^{D} m/ED₅₀. Pharmaceutical compositions that exhibit large therapeutic indices are preferred. The dosage may vary within this range depending upon the dosage form employed, sensitivity of the patient, and the route of administration.

In some embodiments, a drug or its derivatives, drug-polymer conjugates or ADCs (including antibody-drug-polymer conjugates and antibody-drug conjugates) can be evaluated for their ability to inhibit tumor growth in several cell lines using CellTiter Glo®. Dose response curves can be generated using SoftMax Pro software and IC₅₀ values can be determined from four-parameter curve fitting. Cell lines employed can include those which are the targets of the antibody and a control cell line that is not the target of the antibody contained in the test conjugates.

In some embodiments, the PBD conjugates of the disclosure are formulated for parenteral administration by injection including using conventional catheterization techniques or infusion. Formulations for injection may be presented in unit dosage form, e.g., in ampules or in multi-dose containers, with an added preservative. The conjugates can be administered parenterally in a sterile medium. The conjugate, depending on the vehicle and concentration used, can either be suspended or dissolved in the vehicle. Advantageously, adjuvants such as local anesthetics, preservatives, and buffering agents can be dissolved in the vehicle. The term “parenteral” as used herein includes percutaneous, subcutaneous, intravascular (e.g., intravenous), intramuscular, or intrathecal injection or infusion techniques and the like. One or more of the conjugates can be present in association with one or more non-toxic pharmaceutically acceptable carriers and/or diluents and/or adjuvants, and if desired other active ingredients.

The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose, a bland fixed oil can be employed including synthetic mono- or diglycerides. In addition, fatty acids such as oleic acid find use in the preparation of injectables.

The PBD conjugates and compositions described herein may be administered in appropriate form, preferably parenterally, more preferably intravenously. For parenteral administration, the compounds, conjugates or compositions can be aqueous or nonaqueous sterile solutions, suspensions or emulsions. Propylene glycol, vegetable oils and injectable organic esters, such as ethyl oleate, can be used as the solvent or vehicle. The compositions can also contain adjuvants, emulsifiers or dispersants.

For PBD conjugates disclosed herein, the appropriate dosage levels will depend on several factors, such as, In some embodiments, the type of disease to be treated, the severity and course of the disease, whether the compound is administered for preventing or therapeutic purposes, previous therapy, the patient's clinical history. Depending on the type and severity of the disease, about 100 ng to about 25 mg (e.g., about 1 pg/kg to 15 mg/kg, about 0.1-20 mg/kg) of the compound is an initial candidate dosage for administration to the patient, whether, In some embodiments, by one or more separate administrations, or by continuous infusion. A typical daily dosage might range from about 1 pg/kg to 100 mg/kg or more, depending on the factors mentioned above. An exemplary dosage of compound to be administered to a patient is in the range of about 0.1 to about 10 mg/kg of patient weight. For repeated administrations over several days or longer, depending on the condition, the treatment is sustained until a desired suppression of disease symptoms occurs. An exemplary dosing regimen comprises a course of administering an initial loading dose of about 4 mg/kg, followed by additional doses every week, two weeks, or three weeks of a compound. Other dosage regimens may be useful. The progress of this therapy is easily monitored by conventional techniques and assays. Ranges disclosed herein are expressed as amount administered based on the subject's weight, and one skilled in the art can easily express it as amount administered per body surface area of the subject. In some embodiments, 1 mg/kg body weight for a human adult is equivalent to about 37 mg/m² and 1 mg/kg body weight for a human child is equivalent to about 25 mg/m².

For PBD conjugates disclosed herein, dosage levels of the order of from between about 0.01 mg and about 200 mg per kilogram of body weight per day are useful in the treatment of the target conditions (between about 0.05 mg and about 7 g per subject per day). In some embodiments, the dosage administered to a patient is between about 0.01 mg/kg to about 100 mg/kg of the subject's body weight. In some embodiments, the dosage administered to a patient is between about 0.01 mg/kg to about 15 mg/kg of the subject's body weight. In some embodiments, the dosage administered to a patient is between about 0.1 mg/kg and about 15 mg/kg of the subject's body weight. In some embodiments, the dosage administered to a patient is between about 0.1 mg/kg and about 20 mg/kg of the subject's body weight. In some embodiments, the dosage administered is between about 0.1 mg/kg to about 5 mg/kg or about 0.1 mg/kg to about 10 mg/kg of the subject's body weight. In some embodiments, the dosage administered is between about 1 mg/kg to about 15 mg/kg of the subject's body weight. In some embodiments, the dosage administered is between about 1 mg/kg to about 10 mg/kg of the subject's body weight.

The amount of conjugate that can be combined with the carrier materials to produce a single dosage form varies depending upon the host treated and the particular mode of administration. Dosage unit forms can generally contain from between about 0.01 mg and about 200 mg; between 0.01 mg and about 150 mg; between 0.01 mg and about 100 mg; between about 0.01 mg and about 75 mg; or between about 0.01 mg and about 50 mg; or between about 0.01 mg and about 25 mg; of a conjugate. In some embodiments, the PBD compound or conjugate of the disclosure can be administered to a subject in need thereof (e.g., a human patient) at a dose of about 100 mg, 3 times daily, or about 150 mg, 2 times daily, or about 200 mg, 2 times daily, or about 50-70 mg, 3-4 times daily, or about 100-125 mg, 2 times daily.

In some embodiments, the conjugates can be administered are as follows. The conjugates can be given daily for about 5 days either as an i.v., bolus each day for about 5 days, or as a continuous infusion for about 5 days.

Alternatively, the conjugates can be administered once a week for six weeks or longer. As another alternative, the conjugates can be administered once every two or three weeks. Bolus doses are given in about 50 to about 400 ml of normal saline to which about 5 to about 10 ml of human serum albumin can be added. Continuous infusions are given in about 250 to about 500 ml of normal saline, to which about 25 to about 50 ml of human serum albumin can be added, per 24 hour period.

In some embodiments about one to about four weeks after treatment, the patient can receive a second course of treatment. Specific clinical protocols with regard to route of administration, excipients, diluents, dosages, and times can be determined by the skilled artisan as the clinical situation warrants.

It is understood that the specific dose level for a particular subject depends upon a variety of factors including the activity of the specific compound or conjugate, the age, body weight, general health, sex, diet, time of administration, route of administration, and rate of excretion, combination with other active agents, and the severity of the particular disease undergoing therapy.

For administration to non-human animals, the conjugates can also be added to the animal feed or drinking water. It can be convenient to formulate the animal feed and drinking water so that the animal takes in a therapeutically appropriate quantity of the conjugates along with its diet. It can also be convenient to present the conjugates as a premix for addition to the feed or drinking water.

The PBD conjugates disclosed herein can also be administered to a subject in combination with other therapeutic compounds to increase the overall therapeutic effect. The use of multiple compounds to treat an indication can increase the beneficial effects while reducing the presence of side effects. In some embodiments, the conjugates are used in combination with chemotherapeutic agents, such as those disclosed in U.S. Pat. No. 7,303,749, U.S. 2016/0031887 and U.S. 2015/0133435, each of which is herein incorporated by reference by its entirety. In other embodiments, the chemotherapeutic agents, include, but are not limited to letrozole, oxaliplatin, docetaxel, 5-FU, lapatinib, capecitabine, leucovorin, erlotinib, pertuzumab, bevacizumab, and gemcitabine.

The present disclosure also provides pharmaceutical kits comprising one or more containers filled with one or more of the compounds, conjugates and/or compositions of the present disclosure, including, one or more chemotherapeutic agents. Such kits can also include, In some embodiments, other compositions, a device(s) for administering the compositions, and written instructions in a form prescribed by a governmental agency regulating the manufacture, use or sale of pharmaceuticals or biological products.

In another aspect, the PBD conjugates of the disclosure are used in methods of treating animals (preferably mammals, most preferably humans and includes males, females, infants, children and adults).

The conjugates of the disclosure may be used to provide a PBD conjugate at a target location.

The target location is preferably a proliferative cell population. The antibody is an antibody for an antigen present in a proliferative cell population.

In some embodiments, the antigen is absent or present at a reduced level in a non-proliferative cell population compared to the amount of antigen present in the proliferative cell population, for example a tumor cell population.

The target location may be in vitro, in vivo or ex vivo.

The antibody-drug conjugate (ADC) of the disclosure include those with utility for anticancer activity. In particular, the ADC includes an antibody conjugated, i.e. covalently attached by a linker, to a PBD moiety.

At the target location the linker may not be cleaved. The ADC of the disclosure may have a cytotoxic effect without the cleavage of the linker to release a PBD drug moiety. The ADC of the disclosure selectively deliver cytotoxic agent to tumor tissue whereby greater selectivity, i.e., a lower efficacious dose, may be achieved.

In a further aspect, a conjugate as described herein is for use in the treatment of a proliferative disease. A second aspect of the present disclosure provides the use of a conjugate compound in the manufacture of a medicament for treating a proliferative disease.

One of ordinary skill in the art is readily able to determine whether or not a candidate conjugate treats a proliferative condition for any particular cell type. In some embodiments, assays which may conveniently be used to assess the activity offered by a particular compound are described in the examples below.

The term “proliferative disease” pertains to an unwanted or uncontrolled cellular proliferation of excessive or abnormal cells which is undesired, such as, neoplastic or hyperplastic growth, whether in vitro or in vivo.

Examples of proliferative conditions include, but are not limited to, benign, pre-malignant, and malignant cellular proliferation, including but not limited to, neoplasms and tumors (e.g. histiocytoma, glioma, astrocytoma, osteoma), cancers (e.g. lung cancer, small cell lung cancer, gastrointestinal cancer, bowel cancer, colon cancer, breast carcinoma, ovarian carcinoma, prostate cancer, testicular cancer, liver cancer, kidney cancer, bladder cancer, pancreas cancer, brain cancer, sarcoma, osteosarcoma, Kaposi's sarcoma, melanoma), leukemias, psoriasis, bone diseases, fibroproliferative disorders (e.g. of connective tissues), and atherosclerosis. Cancers of particular interest include, but are not limited to, leukemias and ovarian cancers.

Any type of cell may be treated, including but not limited to, lung, gastrointestinal (including, e.g. bowel, colon), breast (mammary), ovarian, prostate, liver (hepatic), kidney (renal), bladder, pancreas, brain, and skin.

In some embodiments, the treatment is of a pancreatic cancer.

In some embodiments, the treatment is of a tumor having αᵥβ₃ integrin on the surface of the cell.

It is contemplated that the ADC of the present disclosure may be used to treat various diseases or disorders, e.g. characterized by the overexpression of a tumor antigen. Exemplary conditions or hyperproliferative disorders include benign or malignant tumors; leukemia, hematological, and lymphoid malignancies. Others include neuronal, glial, astrocytal, hypothalamic, glandular, macrophagal, epithelial, stromal, blastocoelic, inflammatory, angiogenic and immunologic, including autoimmune, disorders.

Generally, the disease or disorder to be treated is a hyperproliferative disease such as cancer. Examples of cancer to be treated herein include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulvar cancer, thyroid cancer, hepatic carcinoma, anal carcinoma, penile carcinoma, as well as head and neck cancer.

Autoimmune diseases for which the ADC compounds may be used in treatment include rheumatologic disorders (such as, In some embodiments, rheumatoid arthritis, Sjögren's syndrome, scleroderma, lupus such as SLE and lupus nephritis, polymyositis/dermatomyositis, cryoglobulinemia, anti-phospholipid antibody syndrome, and psoriatic arthritis), osteoarthritis, autoimmune gastrointestinal and liver disorders (such as, In some embodiments, inflammatory bowel diseases (e.g. ulcerative colitis and Crohn's disease), autoimmune gastritis and pernicious anemia, autoimmune hepatitis, primary biliary cirrhosis, primary sclerosing cholangitis, and celiac disease), vasculitis (such as, In some embodiments, ANCA-associated vasculitis, including Churg-Strauss vasculitis, Wegener's granulomatosis, and polyarteriitis), autoimmune neurological disorders (such as, In some embodiments, multiple sclerosis, opsoclonus myoclonus syndrome, myasthenia gravis, neuromyelitis optica, Parkinson's disease, Alzheimer's disease, and autoimmune polyneuropathies), renal disorders (such as, In some embodiments, glomerulonephritis, Goodpasture syndrome, and Berger's disease), autoimmune dermatologic disorders (such as, In some embodiments, psoriasis, urticaria, hives, pemphigus vulgaris, bullous pemphigoid, and cutaneous lupus erythematosus), hematologic disorders (such as, In some embodiments, thrombocytopenic purpura, thrombotic thrombocytopenic purpura, post-transfusion purpura, and autoimmune hemolytic anemia), atherosclerosis, uveitis, autoimmune hearing diseases (such as, In some embodiments, inner ear disease and hearing loss), Behcet's disease, Raynaud's syndrome, organ transplant, and autoimmune endocrine disorders (such as, In some embodiments, diabetic-related autoimmune diseases such as insulin-dependent diabetes mellitus (IDDM), Addison's disease, and autoimmune thyroid disease (e.g. Graves' disease and thyroiditis)). More preferred such diseases include, In some embodiments, rheumatoid arthritis, ulcerative colitis, ANCA-associated vasculitis, lupus, multiple sclerosis, Sjögren's syndrome, Graves' disease, IDDM, pernicious anemia, thyroiditis, and glomerulonephritis.

The term “treatment,” as used herein in the context of treating a condition, pertains generally to treatment and therapy, whether of a human or an animal (e.g., in veterinary applications), in which some desired therapeutic effect is achieved, In some embodiments, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The subject/patient in need thereof may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutan, gibbon), or a human.

Furthermore, the subject/patient may be any of its forms of development, In some embodiments, a fetus. In one preferred embodiment, the subject/patient is a human.

In some embodiments, the patient is a population where each patient has a tumor having αᵥβ₆ integrin on the surface of the cell.

In certain embodiments, in practicing the method of the present disclosure, the conjugate further comprises or is associated with a diagnostic label. In certain exemplary embodiments, the diagnostic label is selected from the group consisting of: radiopharmaceutical or radioactive isotopes for gamma scintigraphy and PET, contrast agent for Magnetic Resonance Imaging (MRI), contrast agent for computed tomography, contrast agent for X-ray imaging method, agent for ultrasound diagnostic method, agent for neutron activation, moiety which can reflect, scatter or affect X-rays, ultrasounds, radiowaves and microwaves and fluorophores. In certain exemplary embodiments, the conjugate is further monitored in vivo.

Examples of diagnostic labels include, but are not limited to, diagnostic radiopharmaceutical or radioactive isotopes for gamma scintigraphy and PET, contrast agent for Magnetic Resonance Imaging (MRI) (for example paramagnetic atoms and superparamagnetic nanocrystals), contrast agent for computed tomography, contrast agent for X-ray imaging method, agent for ultrasound diagnostic method, agent for neutron activation, and moiety which can reflect, scatter or affect X-rays, ultrasounds, radiowaves and microwaves, fluorophores in various optical procedures, etc. Diagnostic radiopharmaceuticals include γ-emitting radionuclides, e.g., indium-111, technetium-99m and iodine-131, etc. Contrast agents for MRI (Magnetic Resonance Imaging) include magnetic compounds, e.g., paramagnetic ions, iron, manganese, gadolinium, lanthanides, organic paramagnetic moieties and superparamagnetic, ferromagnetic and antiferromagnetic compounds, e.g., iron oxide colloids, ferrite colloids, etc. Contrast agents for computed tomography and other X-ray based imaging methods include compounds absorbing X-rays, e.g., iodine, barium, etc. Contrast agents for ultrasound based methods include compounds which can absorb, reflect and scatter ultrasound waves, e.g., emulsions, crystals, gas bubbles, etc. Still other examples include substances useful for neutron activation, such as boron and gadolinium. Further, labels can be employed which can reflect, refract, scatter, or otherwise affect X-rays, ultrasound, radiowaves, microwaves and other rays useful in diagnostic procedures. Fluorescent labels can be used for photoimaging. In certain embodiments a modifier comprises a paramagnetic ion or group.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

The following working examples are illustrative of the linkers, drug molecules and antibodies or antibody fragments, and methods for preparing same. These are not intended to be limiting and it will be readily understood by one of skill in the art that other reagents or methods may be utilized.

### Abbreviations

The following abbreviations are used in the reaction schemes and synthetic examples, which follow. This list is not meant to be an all-inclusive list of abbreviations used in the application as additional standard abbreviations, which are readily understood by those skilled in the art of organic synthesis, can also be used in the synthetic schemes and examples
ACN Acetonitrile
Alloc Allyloxycarbonyl
AcOH Acetic acid
BAIB Diacetoxyiodo)benzene
DABCO 1,4-Diazabicyclo[2.2.2]octane
DBU 1,8-Diazabicyclo[5.4.0]undec-7-ene
DCE 1,2-Dichloroethene
DCHA 2-Methylindol-1-ylacetic acid
DCM Dichloromethane
DIEA N,N-Diisopropylethylamine
DHP Dihydropyran
DMA N, N-Dimethylacetamide
DMF Dimethylformamide
DMAP 4-Dimethylaminopyridine
EEDQ 2-Ethoxy-1-ethoxycarbonyl-1,2-dihydroquinoline
EDCI N1-((ethylimino)methylene)-N3,N3-dimethylpropane-1,3-diamine hydrochloride
EDTA Ethylenediaminetetraacetic acid
EDC 1-Ethyl-3-[3-dimethylaminopropyl]carbodiimide hydrochloride
HATU 1-[Bis(dimethylamino)methylene]-1H-1,2,3-triazolo[4,5-b]pyridinium 3-oxid hexafluorophosphate
HOAt 1-Hydroxy-7-azabenzotriazole
HOBt Hydroxybenzotriazole
MPLC Medium Pressure Liquid Chromatography
TEA Triethylamine
TEAA Triethylammonium acetate
TEMPO (2,2,6,6-Tetramethylpiperidin-1-yl)oxyl or (2,2,6,6-tetramethylpiperidin-1-yl)oxidanyl
TCEP Tris[2-carboxyethyl] phosphine
THF Tetrahydrofuran
pTSA para-Toluenesulfonic acid
MI Maleimide or maleimido
MTBE Methyl t-butyl ether
MTT 4-Methyltrityl
NHS 1-Hydroxypyrrolidine-2,5-dione (i.e., N-hydroxy-succinimide
NMPN-Methyl-2-pyrrolidone
RP-HPLC Reverse-phase high performance liquid chromatography
SEC Size exclusion chromatography
WCX Weak cation exchange chromatography

### General Information

Tumor growth inhibition (% TGI) was defined as the percent difference in median tumor volumes (MTVs) between treated and control groups.

Treatment efficacy was determined from the incidence and magnitude of regression responses of the tumor size observed during the study. Treatment may cause partial regression (PR) or complete regression (CR) of the tumor in an animal. In a PR response, the tumor volume was 50% or less of its Day 1 volume for three consecutive measurements during the course of the study, and equal to or greater than 13.5 mm³ for one or more of these three measurements. In a CR response, the tumor volume was less than 13.5 mm3 for three consecutive measurements during the course of the study. An animal with a CR response at the termination of a study was additionally classified as a tumor-free survivor (TFS). Animals were monitored for regression responses.

XMT-1535 is disclosed in co-pending application U.S. Ser. No. 15/457,574 filed Mar. 13, 2017.

HPLC purification was performed on a Phenomenex Gemini 5 μm 110 Å, 250×10 mm, 5 micron, semi-preparation column.

Whenever possible the drug content of the conjugates was determined quantitatively by chromatography.

The protein content of the protein-drug conjugates was determined spectrophotometrically at 280 nm or by ELISA.

Antibody-drug conjugates, can be purified (i.e., removal of residual unreacted drug, antibody, or starting materials) by extensive diafiltration. If necessary, additional purification by size exclusion chromatography can be conducted to remove any aggregated antibody-drug conjugates. In general, the antibody-drug conjugates as purified typically contain <5% (e.g., <2% w/w) aggregated antibody-drug conjugates as determined by SEC; <0.5% (w/w) (e.g., <0.1% w/w) free (unconjugated) drug as determined by RP-HPLC or LC-MS/MS; <1% (w/w) of free drug conjugate as determined by SEC and/or RP-HPLC and <2% (w/w) (e.g., <1% w/w) unconjugated antibody or antibody fragment as determined by HIC-HPLC and/or WCX HPLC. Reduced or partially reduced antibodies were prepared using procedures described in the literature, see, for example, Francisco et al., Blood 102 (4): 1458-1465 (2003). The total drug (conjugated and unconjugated) concentration was determined by RP-HPLC or back-calculation from DAR measured by CE-SDS.

RP-HPLC, or CE-SDS were used to characterize the specificity and distribution of the cysteine bioconjugation sites in the PBRM-drug conjugates. The results gave the positional distribution of the drug-conjugates on the heavy (H) and light (L) chains of the PBRM.

To determine the concentration of the free drug in a biological sample, an acidified sample was treated with acetonitrile. The free drug was extracted and the acetonitrile supernatant was analyzed. To determine the concentration of conjugated AF-HPA, the sample was subjected to exhaustive basic hydrolysis followed by immunocapture using anti-IgG1 antibody magnetic beads. The acetonitrile supernatant containing the released AF-HPA and AF was analyzed RP-HPLC. The total antibody was measured using the unique peptide after digestion. Analysis of free AF and AF-HPA was conducted by RP-HPLC using a C-4 column, an acetonitrile gradient and UV detection. Peak areas are integrated and compared to AF and AF-HPA standards. The method is quantitative for AF-HPA and AF in plasma and tissue homogenates and linear over the concentration ranges of 0.1 to 150 ng/mL. The total drug (AF-HPA) released after hydrolysis with NaOH was measured under the same condition with the dynamic range from 1 ng/mL to 5000 ng/mL. The total antibody standards range from 0.1 pg/mL to 100 pg/mL.

### General Procedure A: Partial Selective Reduction of Protein (Antibody)

The partial selective reduction of the inter-chain disulfide groups or unpaired disulfide in the relevant antibody prior to conjugation with the polymer-drug conjugate is achieved by using a reducing agent, such as, In some embodiments, TCEP, DTT or β-mercaptoethanol. When the reduction is performed with an excess of the reducing agent, the reducing agent is removed prior to conjugation by SEC. The degree of conversion of the antibody disulfide groups into reactive sulfhydryl groups depends on the stoichiometry of antibody, reducing agent, pH, temperature and/or duration of the reaction. When some but not all of the disulfide groups in the antibody are reduced, the reduced antibody is a partially reduced antibody.

General Procedure B: Conjugation of Partially Reduced Antibody with Drug Conjugate

The conjugation of the partially reduced antibody to the drug conjugate is conducted under neutral or slightly basic conditions (pH 6.5-8.5) at antibody concentrations of 1-10 mg/mL and drug conjugate concentrations of 0.5-10 mg/mL. The drug conjugate is typically used in 1-5.0 fold excess relative to the desired protein-drug conjugate stoichiometry. When the antibody is conjugated to the maleimido group of the drug conjugate, the conjugation is optionally terminated by the addition of a water-soluble maleimido blocking compound, such as, In some embodiments, N-acetyl cysteine, cysteine methyl ester, N-methyl cysteine, 2-mercaptoethanol, 3-mercaptopropanoic acid, 2-mercaptoacetic acid, mercaptomethanol (i.e., HOCH₂SH), benzyl thiol, and the like.

The resulting antibody-drug conjugate is typically purified by diafiltration to remove any unconjugated polymer-drug conjugate, unconjugated drug and small molecule impurities. Alternatively or additionally, appropriate chromatographic separation procedures such as, In some embodiments, size-exclusion chromatography, hydrophobic interaction chromatography, ion chromatography such as, In some embodiments, WCX chromatography; reversed phase chromatography, hydroxyl apatite chromatography, affinity chromatography or combinations thereof may be used to purify the antibody-drug conjugate. The resulting purified polymer-drug conjugate is typically formulated in a buffer at pH 5.0-6.5.

Other antibody-drug conjugates are synthesized with methods similar to the procedure described herein, involving other antibodies and/or antibody fragments. Also antibody-drug conjugates with varying ratios of drug to antibody are obtained by varying the number of antibody sulfhydryl groups and drug load.

### Example 1: Synthesis of Trastuzumab Conjugate 5

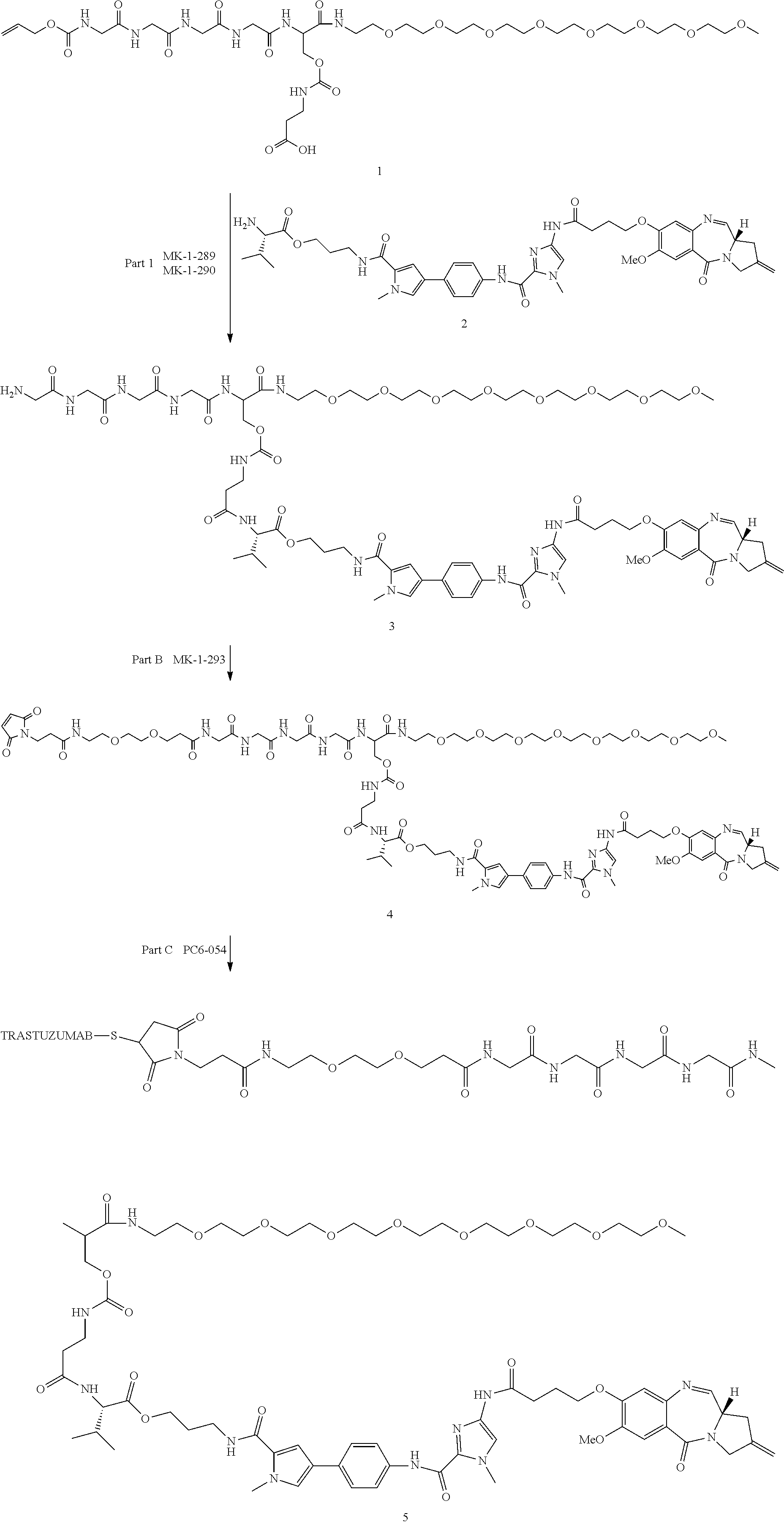

### Part A:

To a solution of compound 1 (7.00 mg, 7.80 μmol, prepared as described in U.S. Ser. No. 15/819,650) in water (300 μL) was added HOAt) (1.59 mg, 0.012 mmol) in NMP (50 μL), then EDC (3.74 mg, 0.019 mmol) at 0° C. was added. The pH of the resulting mixture was adjusted to pH 6-7. To this mixture was added compound 2 (8.12 mg, 9.35 μmol, prepared as described in U.S. Ser. No. 15/630,068) in NMP(200 μL) at 0° C. and the reaction mixture was allowed to warm up to room temperature. After 1.5 h, The reaction mixture was monitored b additional 1 equivalent of HOAt in NMP(50 μL) and EDC in water (100 μL) were added. The reaction mixture was allowed to warm up to room temperature and then stirred overnight. The crude product was purified by RF C18 column CombiFlash (10-70% acetonitrile/water containing 0.1% HOAc) to afford the desired Alloc-protected intermediate alloc-protected intermediate (7 mg, 53%). ESI MS calc for C₇₉H₁₁₃N₁₆O₂₆(M+H) 1701.8; found 1701.7.

To a solution of the Alloc-protected intermediate (7 mg, 4.11 μmol) in degassed CHCl₃/DMF (1:1, 400 μL) was added pyrrolidine (0.68 μL) in CHCl₃ (10 μL). followed by the addition of Pd(PPh₃)₄ (0.2 equivalents) in chloroform (40 μL). the reaction mixture was stirred at room temperature. The crude material was purified by C18 RP HPLC (C-18, 10-70% acetonitrile/water containing 0.1% HOAc) to afford compound 3 (3.7 mg, 55% yield). ESI MS calc for C₇₅H₁₀₈N₁₆O₂₄ [M+H]⁺ 1617.8, found 1617.7.

### Part B:

To a solution of compound 3 (12 mg, 7.42 μmol) in a mixture of NMP((5:2 ratio, 50 μL) and TEA (2.068 μL, 0.015 mmol) was added 2,5-dioxopyrrolidin-1-yl 3-(2-(2-(3-(2,5-dioxo-2,5-dihydro-1H-pyrrol-1-yl)propanamido)ethoxy)ethoxy)propanoate (6.31 mg, 0.015 mmol)) in NMP(50 μL) at 0° C., and the resulting mixture was stirred at room temperature. After 4 hours additional 2,5-dioxopyrrolidin-1-yl 3-(2-(2-(3-(2,5-dioxo-2,5-dihydro-1H-pyrrol-1-yl)propanamido)ethoxy)ethoxy)propanoate (0.7 equivalents) was added and the mixture was stirred overnight. The reaction mixture was neutralized with acetic acid, diluted with water and purified by HPLC (RP C18 column containing 0.1% HOAc (10-70% B over 35 min) to afford compound 4 (3 mg, 21% yield). ESI MS calc for C₈₉H₁₂₆N₁₈O₃₀ (M+2H)964.9, found 964.9.

### Part C:

Conjugate 5 was prepared from Trastuzumab and compound 4 as described in U.S. Ser. No. 15/630,068 The purified conjugate had a PBD to trastuzumab ratio of 5.5 as determined by UV-Vis using molar extinction_{310 nm}=37,500 cm⁻¹ M⁻¹ and ε_{280 nm}=25,394 cm⁻¹ M⁻¹ for compound 2 and ε_{280 nm}=226,107 cm⁻¹ M⁻¹ for trastuzumab).

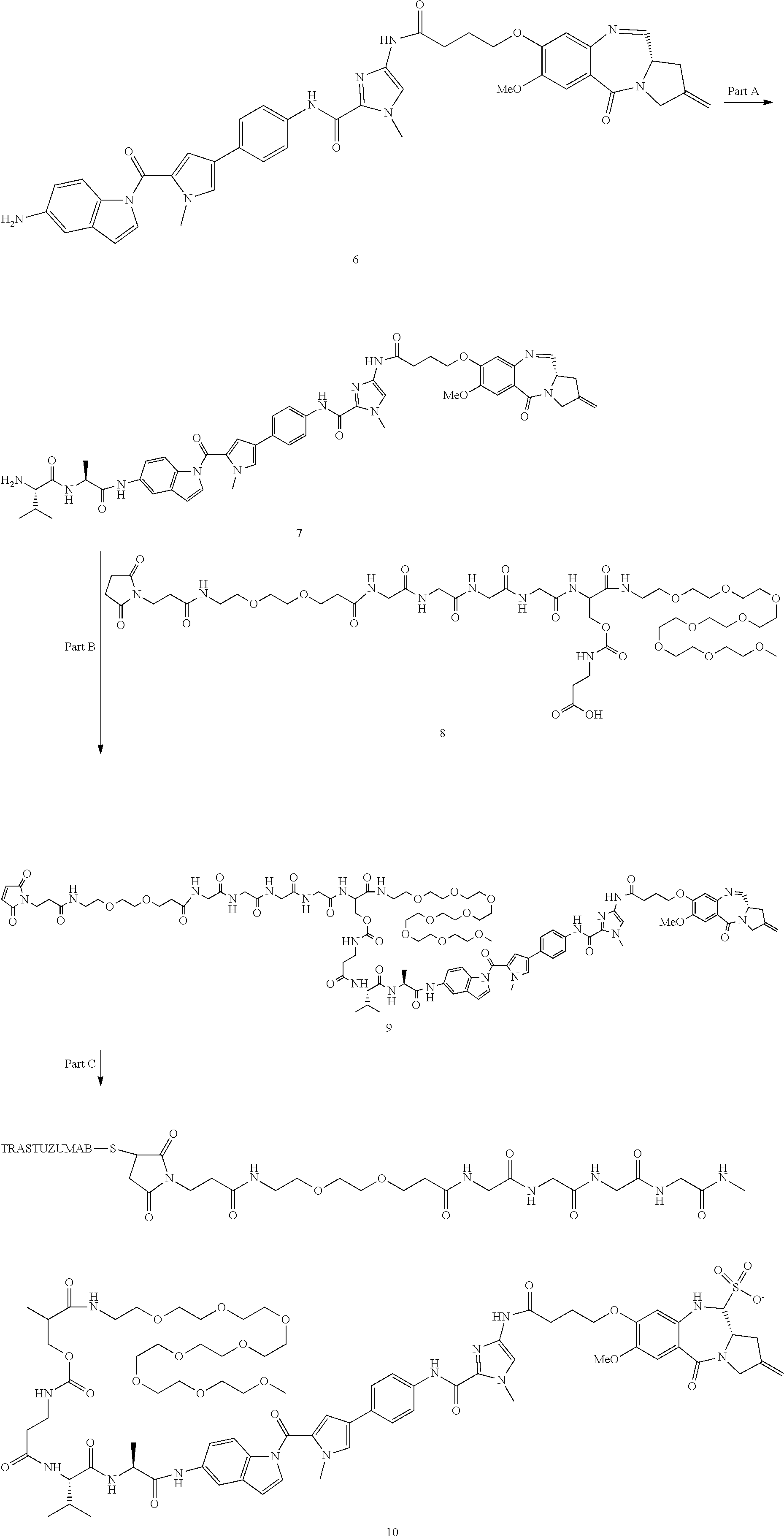

### Part A:

To a solution of Alloc-Val-Ala-OH (25 mg, 0.032 mmol) in THF (1.0 ml) and DMA (0.2 ml) was added compound 6 (10.48 mg, 0.038 mmol, prepared as described in U.S. Ser. No. 15/630,068) and EEDQ. (11.89 mg, 0.048 mmol). The mixture was stirred overnight at room temperature and the crude product was then purified on silica gel (0-15% MeOH/DCM) to afford the desired Alloc-protected intermediate (8 mg, 24.13% yield). ESI MS calc for C₅₅H₆₀N₁₁O₁₀ (M+H) 1034.5; found 1034.5.

To a solution of the Alloc-protected intermediate (8 mg, 7.7 mop in DCM (3 ml) under argon was added triphenylphosphine (0.507 mg, 1.934 μmol) and pyrrolidine (0.800 μl, 9.67 μmol) and the reaction mixture was stirred at room temperature for 10 min, then Pd(PPh₃)₄ (0.447 mg, 0.387 μmol) was added. The resulting solution was stirred at room temperature for 2 h. The crude product was purified on silica gel (0-20% MeOH/DCM) to afford compound 7 (3.6 mg, 49.0% yield), ESI MS calc for C₅₁H₅₆N₁₁O₈ (M+H) 950.4; found 950.4).

### Part B:

To a solution of compound 8 (11.36 mg, 10.10 nmol, prepared as described in U.S. Ser. No. 15/819,650) and compound 7 (6 mg, 6.32 μmol) in NMP (0.7 mL) was added a solution of NHS (1.1 mg, 9.5 μmol) in NMP (46 μL), EDC HCl (1.8 mg, 9.5 μmol) and DIEA (1.2 mg, 9.5 μmol) in NMP (40 μL). The resulting mixture was stirred overnight at room temperature. The crude reaction mixture was purified by RP HPLC (10-90% gradient acetonitrile/water buffered with 0.1% HCO2H) to afford compound 8 as a fluffy solid (6.8 mg, 52% yield).

### Part C:

To Trastuzumab (15 mg, 0.103 μmol) in TEAA buffer (0.757 mL, 50 mM TEAA buffer containing 1 mM EDTA, pH 7.0) was added TCEP (59 μL, 1.0 mg/mL in TEAA buffer) while stirring. The mixture was incubate for ˜90 min at 37° C. with shaking, then cooled to room temperature and diluted with TEAA buffer (1.5 mL). A solution of compound 9 (2.121 mg, 1.032 μmol) in propylene glycol was added. After 1 h at room temperature the reaction was quenched with NALSO₃ (19 TEAA buffer at 27.3 mg/mL). The resulting conjugate was purified by WCX chromatography (Mobile phase A: 20 mM MES, 0.25 mM NaHSO₃, pH 5.8; Mobile phase B: 20 mM MES, 0.25 mM NaHSO₃, 300 mM NaCl, pH 5.8; eluant 20-50%13). The purified conjugate had a PBD to trastuzumab ratio of 4.8 as determined by UV-Vis using molar extinction ε_{330 nm}=38,858.5 cm⁻¹M⁻¹ and ε_{280 nm}=29,820.413 cm⁻¹M⁻¹ for compound 9 and ϵ_{280 nm}=226,107 cm⁻¹M⁻¹ for trastuzumab).

### Example 2A: Synthesis of Trop-2 Conjugate 10A

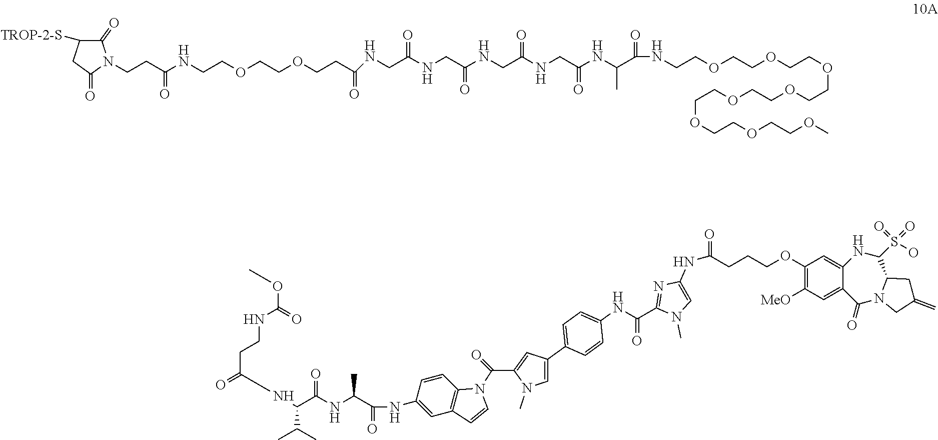

Conjugate NA was prepared as described in Example 2 except that anti-Trop2 antibody was used instead of Trastuzumab. The purified conjugate 10A had a PBD to anti-Trop2 antibody ratio of 5.4 as determined by UV-Vis using molar extinction ε330 nm=38858.5 cm⁻¹ M⁻¹ and ε280 nm 29820.413 cm⁻¹M⁻¹ for compound 9 and ε280 nm 226,372.2 cm⁻¹M⁻¹ for Anti-Trop2 antibody.

### Example 3: Synthesis of Trastuzumab Conjugate 20

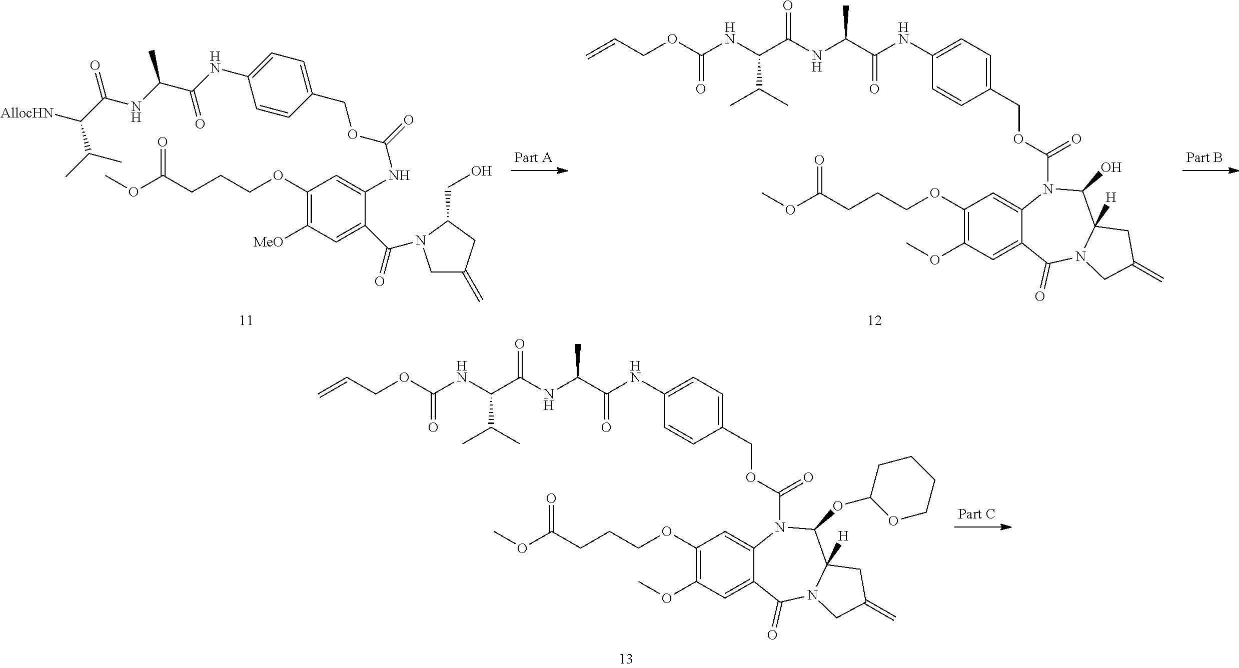

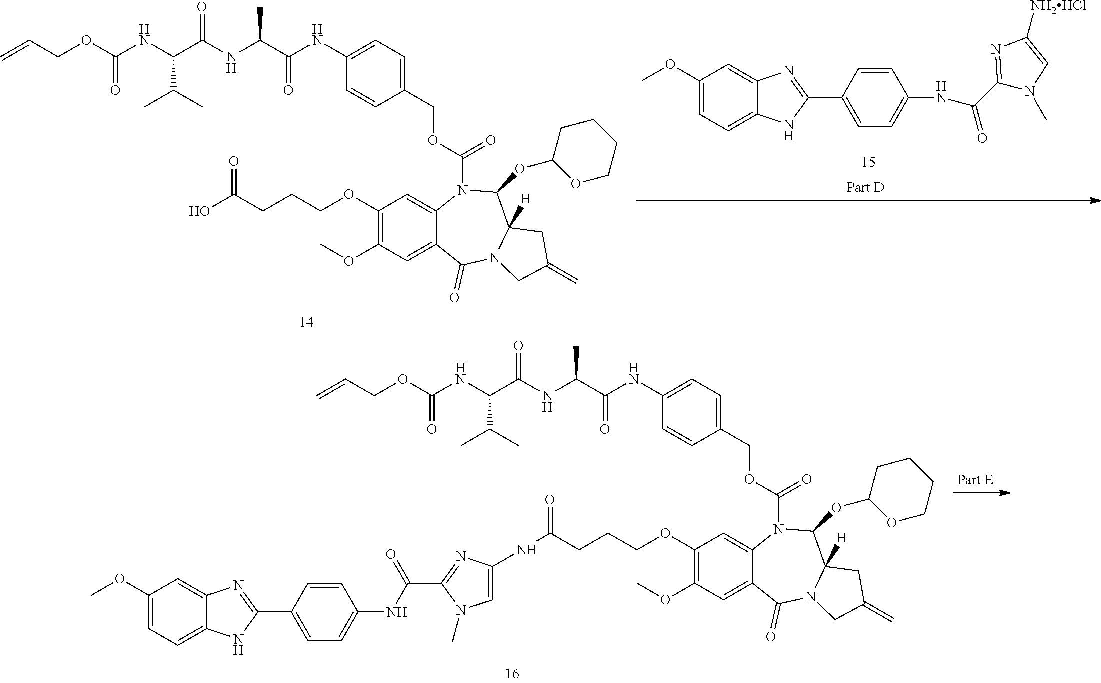

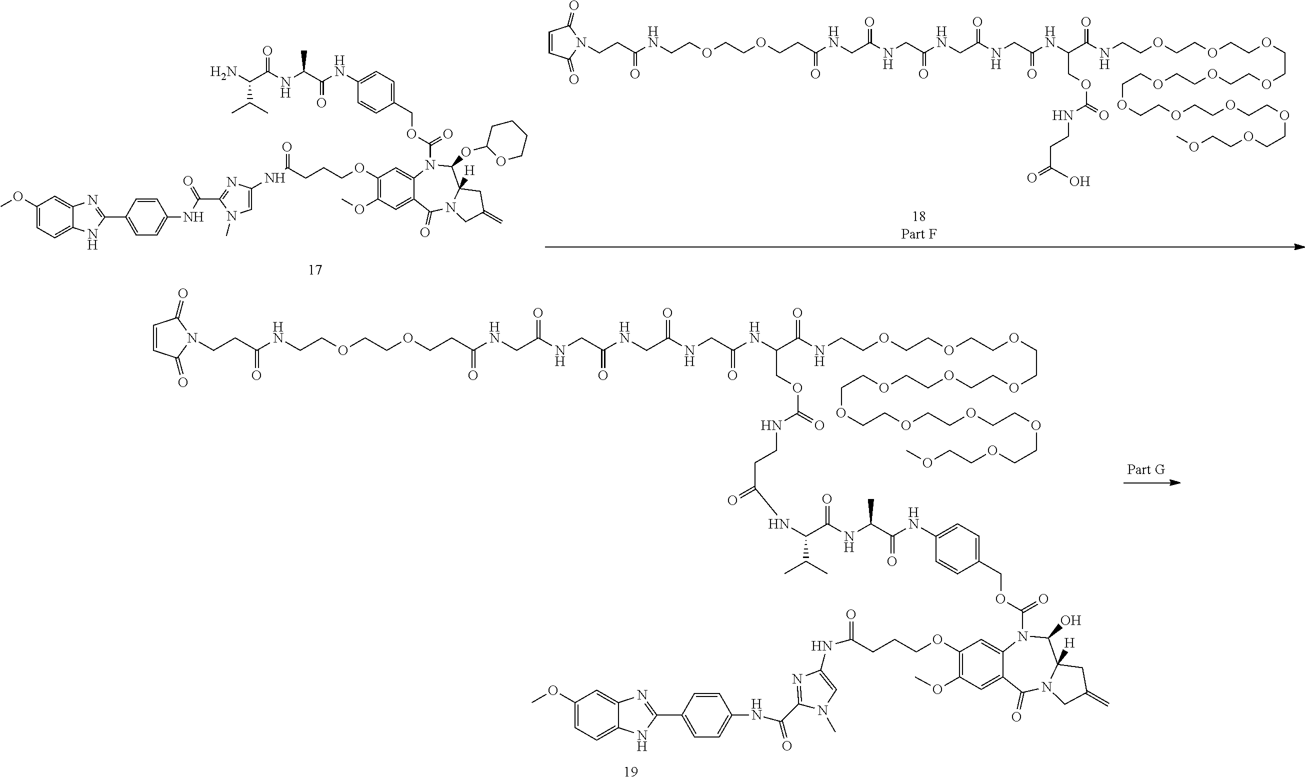

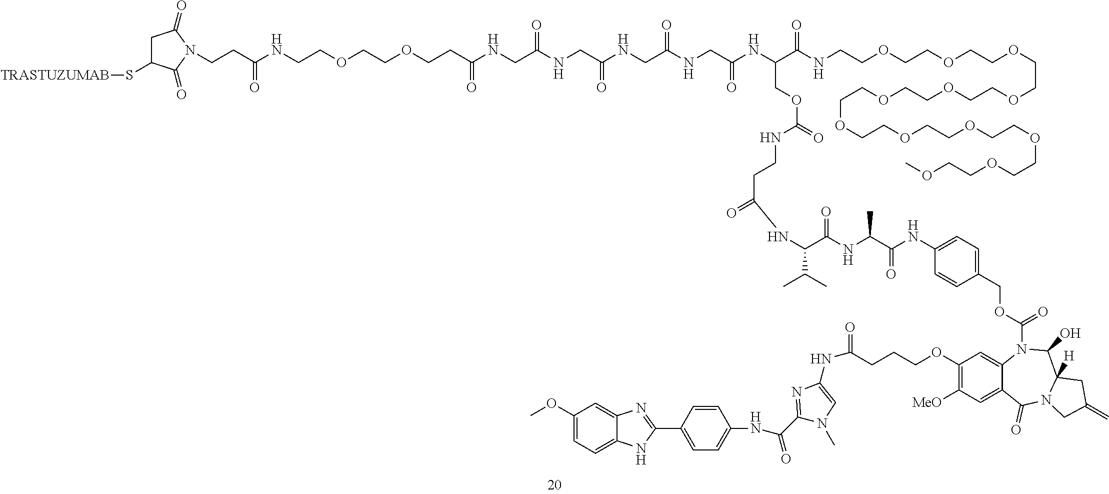

### Part A:

To compound 11 (551 mg, 0.705 mmol, prepared as described in U.S. Ser. No. 15/630,068) under argon was added dichloromethane (7.1 mL) and the solution was stirred at room temperature for 30 min, then BAIB (350 mg, 1.09 mmol) and TEMPO (11 mg, 71 μmol) were added. After 16 h, the crude mixture was purified by chromatography (ISCO, 12 g column, 100% EtOAc eluent) to give compound 12 as a white foam (431 mg, 78% yield). ESI MS calc for C₃₉H₅₀N₅O₁₂ (M+H) 780.4; found 779.9.

### Part B:

Compound 12 (539 mg, 691 μmol), THF (22 mL), pTSA.H₂O (50 mg, 291 μmol) and DHP (2.2 mL, 24.1 mmol) were stirred at room temperature for 2 h. The reaction mixture was evaporated, then the residue dissolved in EtOAc was washed with saturated NaHCO₃ solution and brine. The organic phase was dried over Na₂SO₄, filtered and evaporated to yield a brown oil. This crude product was purified by chromatography (ISCO, 0-20% MeOH/EtOAc eluent) to give compound 13 as a brown foam (514 mg, 86% yield). ESI MS calc for C₄₄H₅₈N₀₁n (M+H) 864.4; found 864.0.

### Part C:

To a solution of compound 13 (512 mg, 593 μmol) in THF (103 mL) was added an aqueous solution of LiOH (0.05 M, 103 mL). The solution was stirred at room temperature for 1 h and then concentrated to remove THF, followed by adjustment of the pH to 4 using HCl (10% aqueous). The aqueous was washed with EtOAc (2×) and the combined orgs washed with brine. The orgs were then dried (Na₂SO₄), filtered and evaporated to yield a brown foam. This crude was then was purified by chromatography (ISCO, 12 g column, 0-10% MeOH/DCM eluent), affording compound 14 as a tan foam (308 mg, 362 μmol, 61% yield). ESI MS calc for C₄₃H₅₆N₅O₁₃ (M+H) 850.4; found 849.9.

### Part D:

Compound 14 (40 mg, 47 μmol), EDCI.HCl (18 mg, 94 μmol), DMAP (17 mg, 141 μmol), DIEA (49 μL, 282 μmol) and DCM (1 mL) were stirred at room temperature for 15 min. Then compound 15 (19 mg, 47 μmol, prepared as in U.S. Ser. No. 15/630,068) was added and the reaction stirred at room temperature for 11 h. The reaction mixture was diluted with DCM, washed with water (2×) and saturated NaHCO₃ solution (2×), dried over Na₂SO₄, concentrated to yield a yellow oil that was purified by chromatography (ISCO, 4 g column, 0-10% MeOH/DCM eluent), to afford compound 16 as a tan solid (32 mg, 57% yield). ESI MS calc for C₆₂H₇₂N₁₁O₁₄ (M+H) 1194.5; found 1194.0.

### Part E:

A solution of compound 16 (32 mg, 27 μmol), DABCO (15 mg, 135 μmol), Pd(PPh₃)₄ (3 mg, 3 μmol) and DCM (1 mL) was stirred at room temperature for 30 min. The reaction mixture was then purified by chromatography (ISCO, 4 g column, 0-10% MeOH/DCM eluent) to afford compound 17 as a yellow powder (15 mg, 50% yield). ESI MS calc for C₅₈H₆₈N₁₁O₁₂ (M+H) 1110.5; found 1109.9.

### Part F:

A solution of compound 18 (23 mg, 14 μmol, prepared as described in U.S. Ser. No. 15/819,650), EDCI.HCl (4 mg, 20 μmol), NHS (2 mg, 20 μmol), DIEA (3.5 μL, 20 μmol) and DMF (0.8 mL) was stirred at room temperature for 15 min followed by the addition of compound 17 (15 mg, 14 μmol). The resulting mixture was stirred at room temperature for 18 h and then evaporated under high vacuum to give the crude product as a yellow gum that was treated a mixture of acetonitrile (54 μL), water (544 μL) and acetic acid (86 μL) and followed by TFA (43 μL) and purified by HPLC (10-100% acetonitrile/water containing 0.1% HCOOH) to afford compound 19 as a fluffy solid (6.3 mg, 2.7 μmol, 20% yield). ESI MS calc for C₁₀₇H₁₅₂N₂₀O₃₇ (M+2H) 1154.5; found 1154.9.

### Part G:

Conjugate 20 was prepared from Trastuzumab and compound 19 as described in Example 2, except the reaction was quenched with cysteine instead of NaHSO₃. The purified conjugate 20 had a PBD to trastuzumab ratio of 3.4 as determined by UV-Vis using molar extinction ϵ_{338 nm}=24,443.8 cm⁻¹ M⁻¹ and ε_{280 nm}=10,584 cm⁻¹ M⁻¹ for compound 19 and ε_{280 nm}=226,107 cm⁻¹ M⁻¹ for trastuzumab).

### Example 3A: Synthesis of XMT-1535 Conjugate 20A

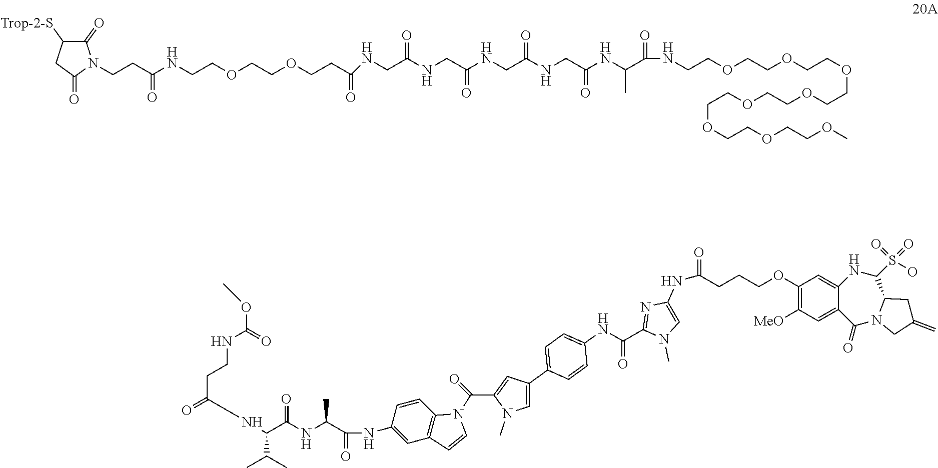

Conjugate 20A was prepared as described in Example 3 except that XMT-1535 antibody was used instead of Trastuzumab and the PEG8 derivative of compound 18 was used instead of compound 18. The purified conjugate 20A had a PBD to XMT-1535 ratio of 3.3.

### Example 4: Synthesis of Trastuzumab Conjugate 26

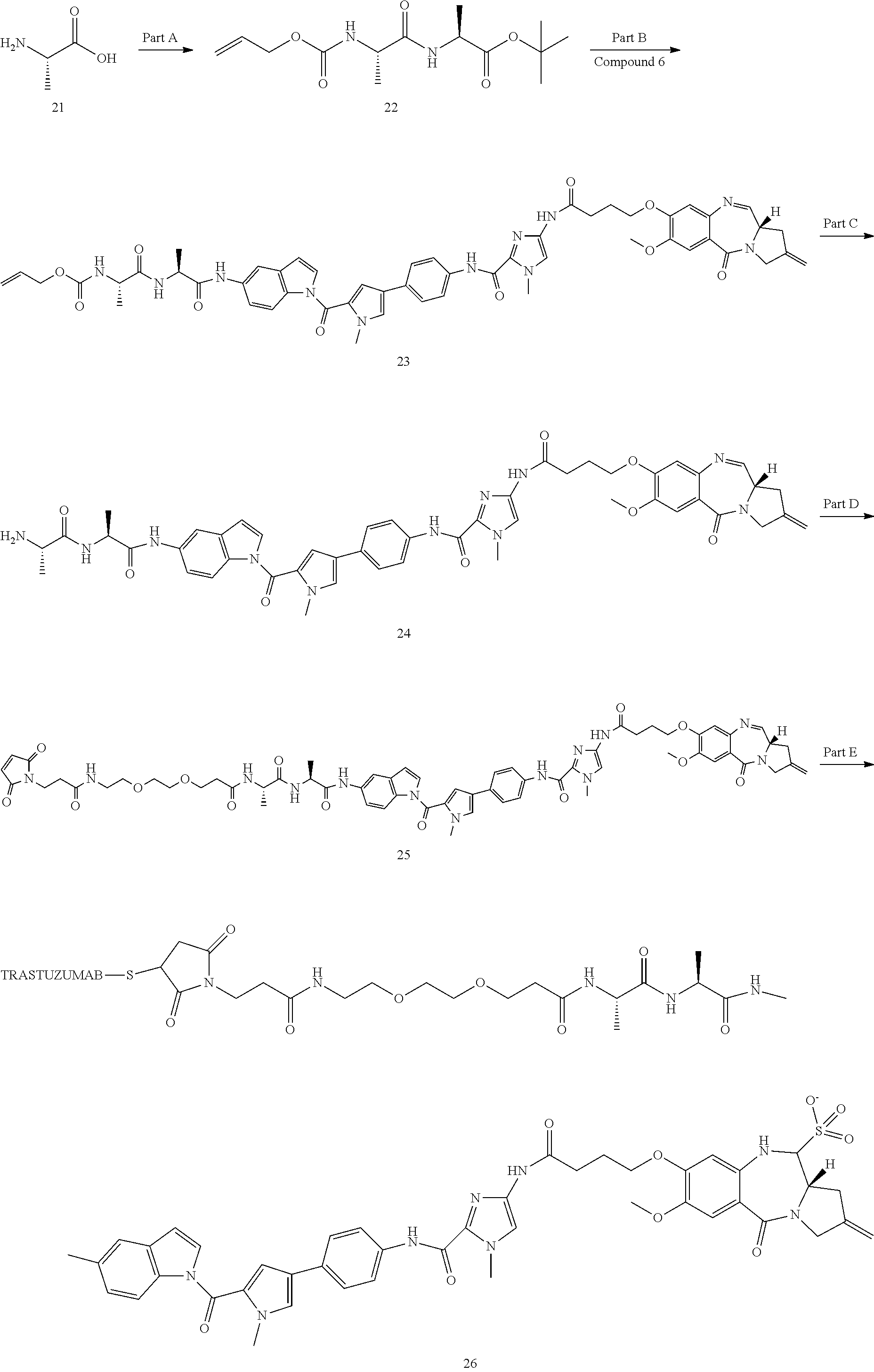

### Part A:

To a solution of the L-alanine (1 g, 11.2 mmol) and K₂CO₃ (3.1 g) in water (15 mL) at 0° C. was added a solution of Alloc-OSu (1.1 eqs, 2.21 g) in THF (15 mL). The resulting mixture was allowed to warm slowly to room temperature and stir overnight. The reaction mixture was concentrated, washed with ether (2×), the pH then adjusted from 11 to ˜3, washed with EtOAc (3×) and the combined organic layers were dried over Na₂SO₄, evaporated to afford Alloc-alanine-OH as a clear oil (2.14 g, 100% yield).

To a mixture of Alloc-alanine-OH (100 mg, 578 μmol), Alanine Me ester.HCl (1 eq, 105 mg), HOAt (1 eq, 79 mg) in DMF (5 mL) was added TEA (4.5 eqs, 363 μL) and the resulting solution was stirred 5 minutes, followed by the addition of HATU (1.3 eqs, 286 mg). After stirring overnight at room temperature, DMF was removed under vacuum. The residue in EtOAc was washed with water (3×), brine, dried over Na₂SO₄ and concentrated to yield an off-white solid that was triturated in EtOAc to afford compound 22 as a brown oil, (138 mg, 80% yield). 1H NMR (CDCl₃): δ 6.45 (1H, d, J=6.7 Hz), δ 5.98-5.85 (1H, m), δ 5.36-5.26 (2H, m), δ 5.26-5.18 (1H, m), δ 4.57 (2H, d, 1=5.9 Hz), δ 4.48-4.38 (1H, m), δ 4.28-4.16 (1H, m), δ 1.47 (9H, s), 1.43-1.35 (6H, m).

### Part B:

Compound 22 (138 mg, 459 μmol) was treated with a mixture of DCM (1.4 mL) and TFA (1.4 mL) overnight at room temperature. The reaction mixture was concentrated under vacuum to afford a yellow gum. Residual TFA was removed to afford the desired Alloc-Ala-Ala free acid intermediate in quantitative yield. 1H NMR (CDCl₃): δ 6.93 (1H, brs), δ 5.99-5.81 (1H, m), δ 5.58 (1H, brs), δ 5.36-5.26 (1H, m), δ 5.26-5.18 (1H, m), δ 4.62-4.52 (3H, m), δ 4.40-4.23 (1H, m), δ 1.47 (3H, d, J=7.1 Hz), δ 1.40 (3H, d, J=6.8 Hz).

The Alloc-Ala-Ala-OH (120 mg, 154 μmol) was dissolved in a solution of THF (3.2 mL) and DMF (648 μL) followed by the addition of compound 6 (46 mg, 185 μmol) and EEDQ (65 mg, 262 μmol). The reaction mixture was stirred at room temperature for 23 h, then concentrated to afford crude compound 23 as a yellow oil that was used in the next step (Part C) without further purification. ESI MS calc for C₅₃H₅₆N₁₁O₁₀ (M+H) 1006.4; found 1006.4.

### Part C:

To a solution of crude compound 23 (154 μmol, 200 mg) in DCM (10 mL) was added DABCO (86 mg, 770 μmol), Pd(PPh₃)₄ (18 mg, 15 μmol) and the resulting mixture was stirred at room temperature for 35 min. The reaction mixture was concentrated under vacuum and the residue was purified on silica gel (ISCO, 12 g column, O—10% MeOH/DCM eluent), to yield compound 24 as a yellow powder (34 mg, 24% yield). ESI MS calc for C₄₉H₅₂N₁₁O₈ (M+H) 922.4; found 922.4.

### Part D:

To a mixture of compound 24 (34 mg, 37 μmol) in DMF (1 mL) was added 2,5-dioxopyrrolidin-1-yl 3-(2-(2-(3-(2,5-dioxo-2,5-dihydro-1H-pyrrol-1-yl)propanamido)ethoxy)ethoxy)propanoate (17 mg, 41 μmol) and TEA (6 μL, 41 μmol) and the resulting mixture was stirred under argon for 1 h. The reaction mixture was concentrated and purified by HPLC (10-100% acetonitrile/water containing 0.1% HCOOH eluent), to afford compound 25 as an off-white, fluffy solid (18 mg, 15 μmol, 40% yield). ESI MS calc for C₆₃H₇₀N₁₃O₁₄ (M+H) 1232.5; found 1232.5.

### Part E:

Conjugate 26 was prepared from Trastuzumab and compound 25 as described in Example 2. The purified conjugate 26 had a PBD to trastuzumab ratio of 4.8 as determined by UV-Vis using molar extinction ε_{330 nm}==38,858.5 cm⁻¹ M⁻¹ and ε_{280 nm}=29,820.413 cm⁻¹ M⁻¹ for compound 9 and ε_{280 nm}=226,107 cm⁻¹ M⁻¹ for trastuzumab).

### Example 5: Synthesis of Trastuzumab Conjugate 31

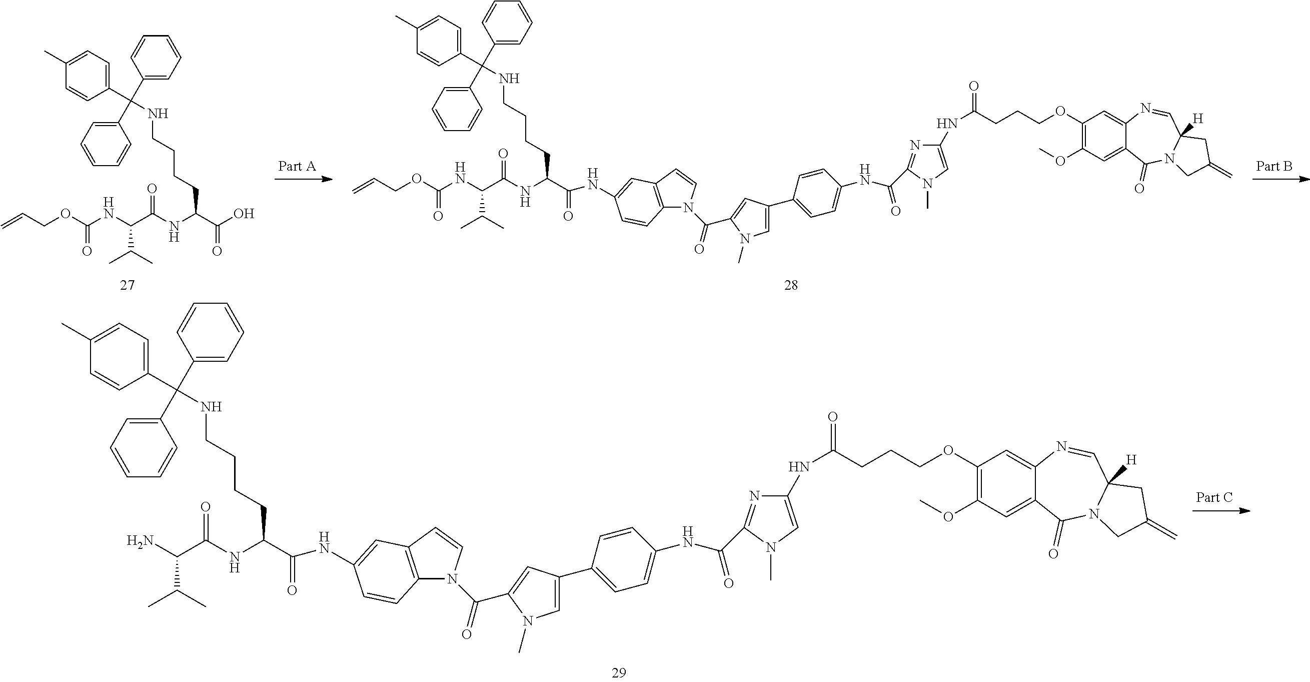

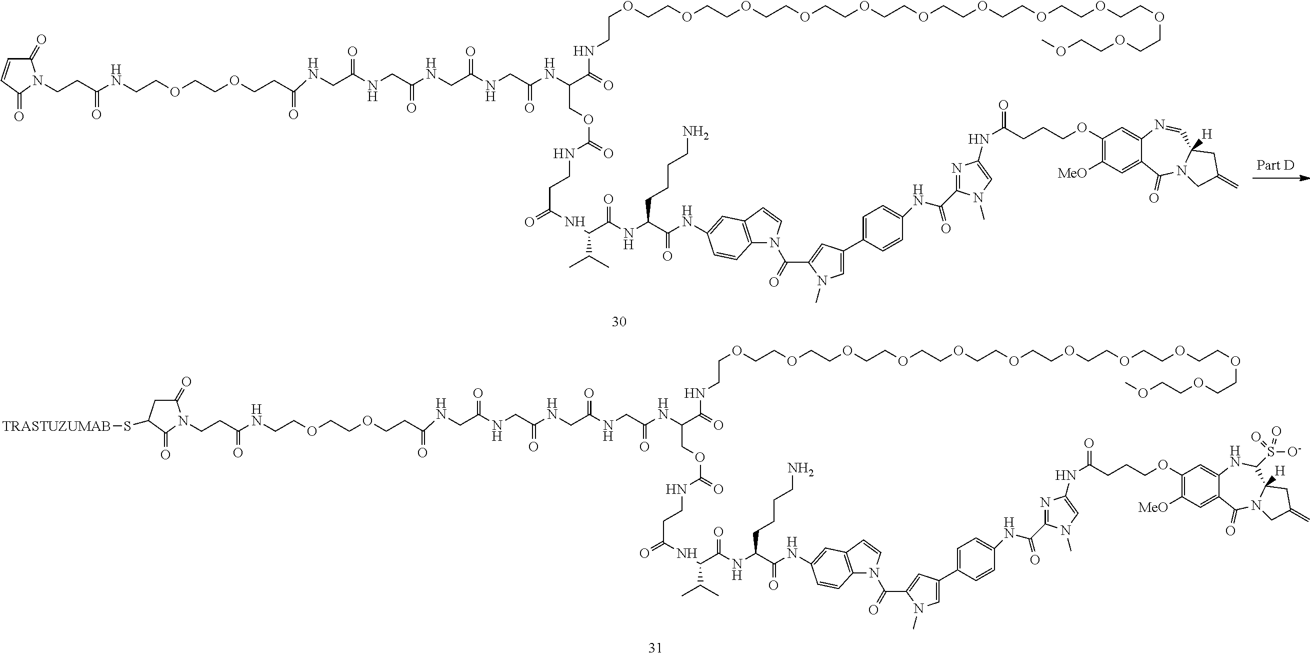

### Part A:

To a solution of compound 6 (60 mg, 77 μmol), THF (1.6 mL) and DMF (324 μL) was added compound 27 (54 mg, 92 μmol) and EEDQ (32 mg, 131 μmol). The reaction mixture was stirred at room temperature for 24 h, then concentrated under vacuum to afford crude compound 28. This material was used in the next step (Part B) without purification. ESI MS calc for C₇₈H₈₃N₁₂O₁₀ (M+H) 1347.6; found 1347.6.

### Part B:

To a solution of crude 28 (77 μmol) in DCM (10 mL) was added DABCO (43 mg, 385 μmol) and Pd(PPh₃)₄ (9 mg, 8 μmol). The resulting mixture was stirred at room temperature for 30 minutes, concentrated and purified on silica gel (ISCO, 4 g column, 0-20° % MeOH/EtOAc eluent), to afford compound 29 as a yellow powder (25 mg, 26% yield). ESI MS calc for C₇₄H₇₉N₂O₈ (M+H) 1263.6; found 1264.4.

### Part C:

Compound 30 was prepared as described above in Example 2 except that compound 18 was used instead of compound 8 to afford compound 30 as a pale yellow solid (5.3 mg, 41.8% yield). ESI MS calc for C₁₀₈H₁₅₅N₂₁O₃₄ (M+2H) 1145.1; found 1145.4.

### Part D:

Conjugate 31 was prepared Trastuzumab and compound 30 as described in Example 2. The purified conjugate 31 had a PBD to trastuzumab ratio of 4.1 as determined by UV-Vis using molar extinction ε_{330 nm}=38,858.5 cm⁻¹ M⁻¹ and ε_{820 nm}=29,820.413 cm⁻¹ M⁻¹ for compound 9 and Σ_{280 nm}=226,107 cm⁻¹ M⁻¹ for trastuzumab).

### Example 6: Synthesis of Trastuzumab Conjugate 36

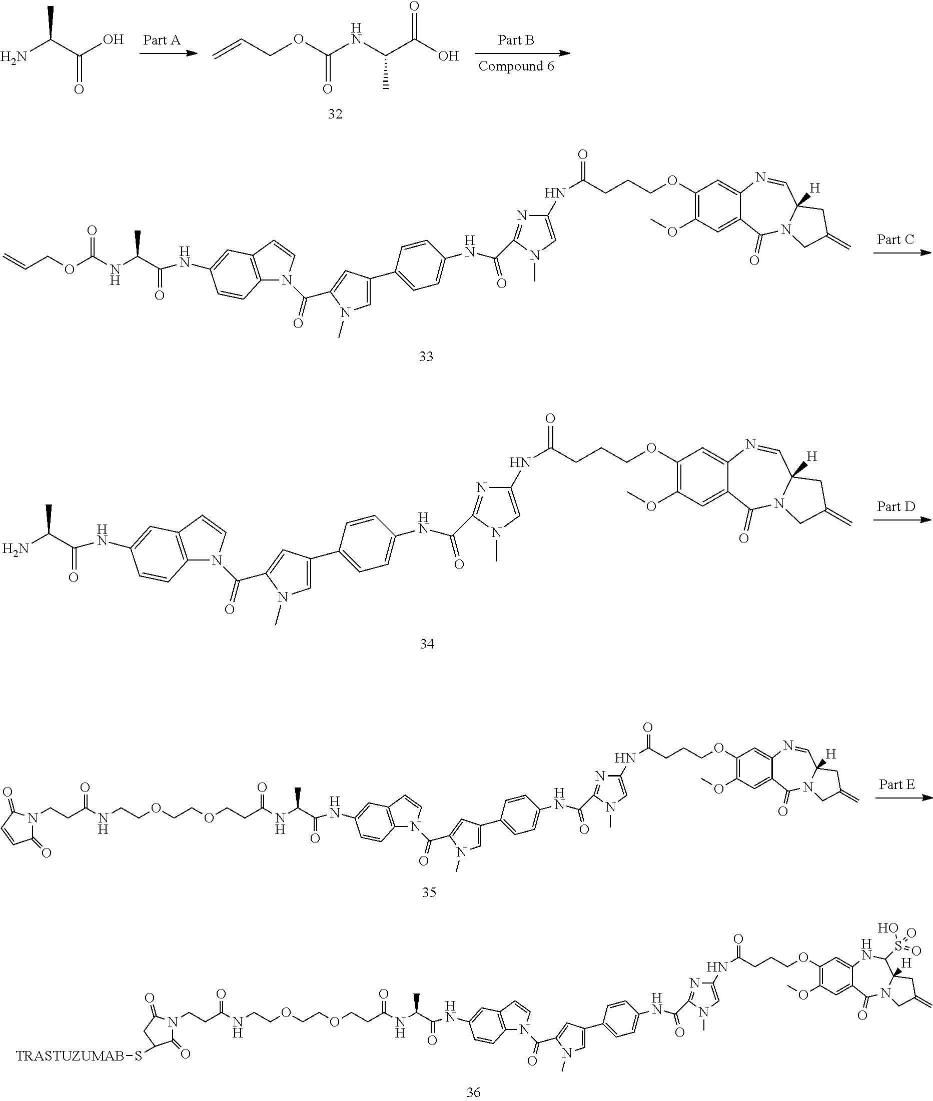

### Part A:

To a solution of the L-alanine (1 g, 11.2 mmol) and K₂CO₃ (3.1 g) in water (15 mL) at 0° C. was added a solution of Alloc-OSu (1.1 eqs, 2.21 g) in THF (15 mL). The resulting mixture was allowed to warm slowly to room temperature and stir overnight. The reaction mixture was concentrated, washed with ether (2×), the pH then adjusted from 11 to ˜3, washed with EtOAc (3×) and the combined organic layers were dried over Na₂SO₄, evaporated to afford compound 32 as a clear oil (2.14 g, 100% yield).

### Part B:

To a solution of the compound 32 (50 mg, 64 μmol) in THF (1.3 mL) and DMF 270 μL) was added compound 6 (1.2 eqs, 13 mg) and EEDQ (1.7 eqs, 27 mg). The reaction was stirred at room temperature for 2 days, then concentrated to afford compound 33 as a yellow oil that was used in the next step without further purification. ESI MS calc for C₅₀H₅₁N₁₀O₉ (M+H) 935.4; found 935.3.

### Part C:

To a solution of compound 33 (64 μmol, 75 mg) in DCM (3.8 mL), was added DABCO (5 eqs, 36 mg) and Pd(PPh₃)₄ (0.1 eqs, 7 mg) and the resulting mixture was stirred at room temperature for 25 min, concentrated and purified by chromatography (ISCO, 4 g column, 0-10% MeOH/DCM eluent) to afford compound 34 as a yellow powder (16 mg, 29% yield). ESI MS calc for C₄₆H₄₇N₁₀O₇ (M+H) 851.4; found 851.1.

### Part D:

To a solution of compound 34 (16 mg, 19 μmol) in DMF (1 mL) was added 2,5-dioxopyrrolidin-1-yl 3-(2-(2-(3-(2,5-dioxo-2,5-dihydro-1H-pyrrol-1-yl)propanamido)ethoxy)ethoxy)propanoate (1.1 eqs, 9 mg) and TEA (1.1 eqs, 3 μL) and the resulting mixture was stirred under argon under for 1.25 h. The reaction mixture was concentrated under vacuum and the residue purified by HPLC to afford compound 35 as a fluffy white solid (10 mg, 46% yield). ESI MS calc for C₆₀H₆₅N₂O₁₃ (M+H) 1161.5; found 1161.4.

### Part E:

Conjugate 36 was prepared from Trastuzumab and compound 35 as described in Example 2. The purified conjugate 36 had a PBD to trastuzumab ratio of 4.5 as determined by UV-Vis using molar extinction ε_{330 nm}=38,858.5 cm⁻¹ M⁻¹ and ε_{280 nm}=29,820.413 cm⁻¹M⁻¹ 1 for compound 9 and ε_{280 nm}=226,107 cm⁻¹M⁻¹ 1 for trastuzumab).

### Example 7: Synthesis of Trastuzumab Conjugate 38

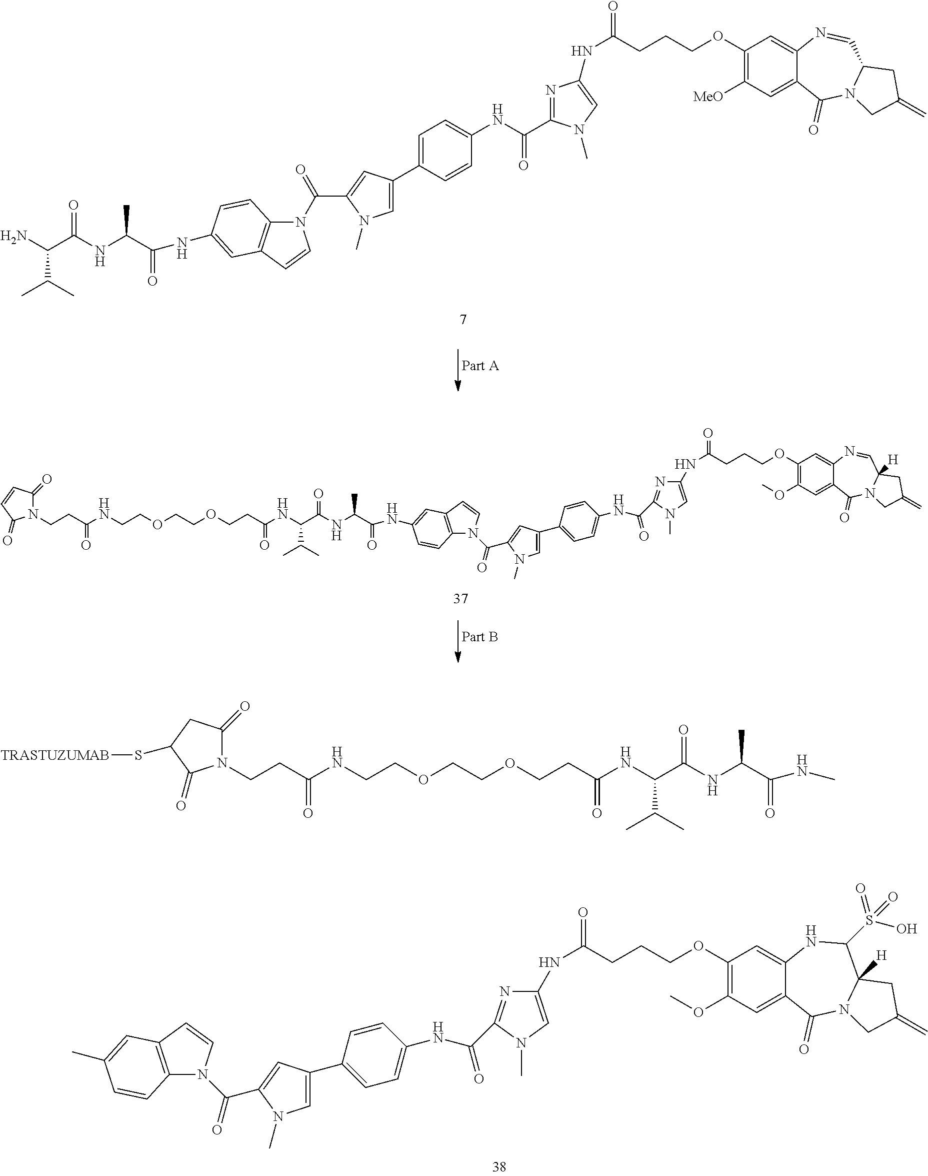

### Part A:

To a solution of compound 7 (18 mg, 14 μmol) in DMF (1 mL) was added 2,5-dioxopyrrolidin-1-yl 3-(2-(2-(3-(2,5-dioxo-2,5-dihydro-1H-pyrrol-1-yl)propanamido)ethoxy)ethoxy)propanoate (7 mg, 15 μmol) and TEA (2 μL, 15 μmol) and the reaction mixture was stirred under argon for 1.5 h. The mixture was then concentrated under vacuum and purified by chromatography (ISCO RP-HPLC, 5.5 g column, 10-100% ACN/water w/0.1% HCOOH eluent) to afford compound 37 as a tan, fluffy solid (17 mg, 13 μmol, 71% yield). ESI MS calc for COH74NisO4 (M+H) 1260.6; found 1261.4.

### Part B:

Conjugate 38 was prepared from Trastuzumab and compound 37 as described in Example 2. The purified conjugate 38 had a PBD to trastuzumab ratio of 3.8 as determined by UV-Vis using molar extinction ε_{330 nm}=38,858.5 cm⁻¹M⁻¹ 1 and ε_{210 nm}=29,820.413 cm⁻¹ M⁻¹ for compound 9 and ε_{280 nm}=226,107 cm⁻¹ M⁻¹ for trastuzumab).

### Example 8: Synthesis of Trastuzumab Conjugate 46

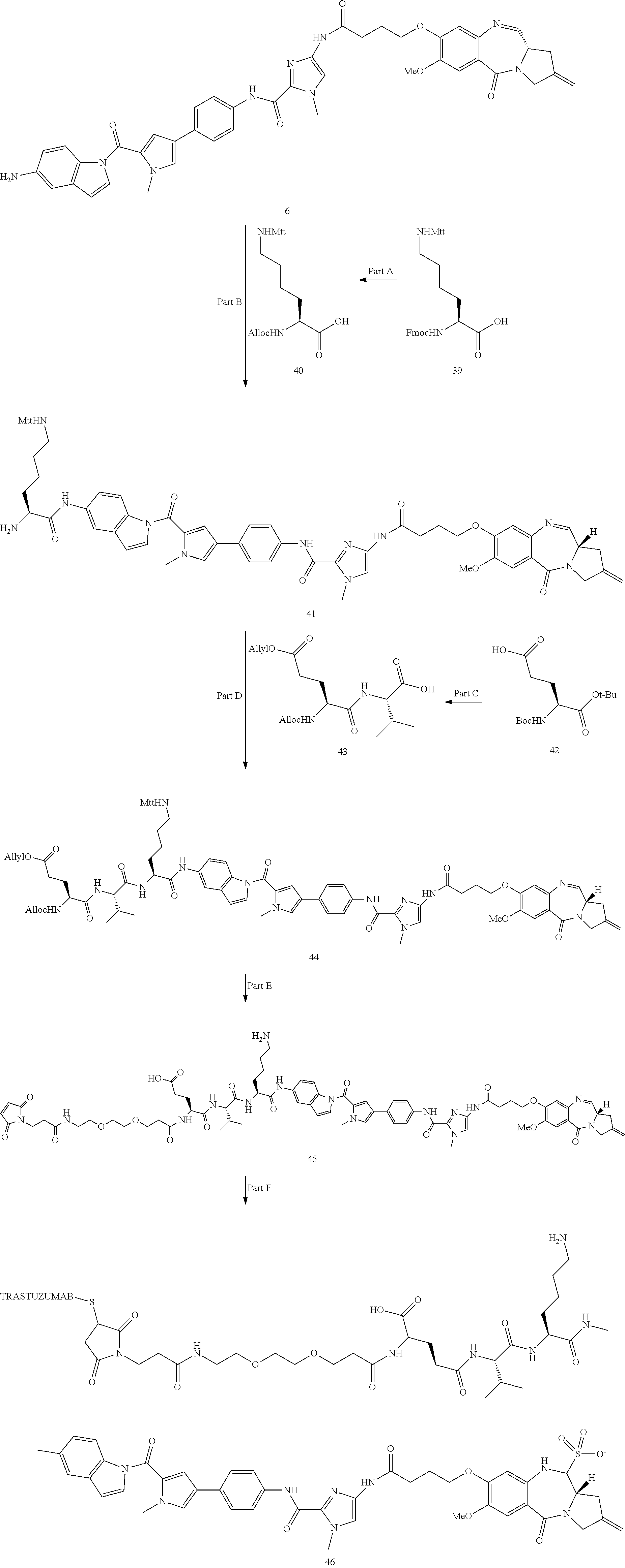

### Part A:

To compound 39 (2.0 g, 3.20 mmol) dissolved in DCM (32 mL) was added piperidine (1.265 mL, 1280 mmol) and stirred for 12 hours at room temperature. The crude reaction mixture was concentrated, then NaH—CO₃ (1.613 g, 19.20 mmol), acetone (80 mL) and water (80 mL) were added followed by the addition of allyl pyrrolidin-1-yl carbonate (2.74 g, 16.00 mmol) and the resulting mixture was stirred for 12 hours at room temperature. The crude reaction mixture was concentrated, then diluted with 100 mL of H₂O (100 mL) and EtOAc (100 mL) followed by glacial HOAc to acidify the mixture to pH 5. The aqueous layer was extracted with EtOAc (3×), the combined organic layers were dried over Na₂SO₄, concentrated and purified on silica gel (0-20% MeOH in DCM) to provide compound 40 (1.15 g, 73.9% yield). ESI-MS: calc. for C₃₀H₃₃N₂O₄⁻ (M−H) 485.2; found 485.2.

### Part B:

To compound 40 (0.1345 g, 0.276 mmol) was added EEDQ (0.092 g, 0.372 mmol), THF (7.29 mL) and DMF (1.458 mL). The resulting solution was added to compound 6 (0.1705 g, 0.219 mmol. The reaction mixture was stirred at room temperature for 72 hours. concentrated, and purified on silica gel (0-15% MeOH in DCM) to provide Alloc protected compound 41 (0.242 g, 89% yield). ESI-MS: calc. for C₇₃H₇₆NiOio (M+H₂O+H) 1266.6; found 1266.6.

To Alloc protected compound 41(0.242 g, 0.194 mmol) was added triphenylphosphine (0.013 g, 0.048 mmol), pyrrolidine (0.020 mL, 0.242 mmol) and DCM (9.69 mL) followed by the addition of tetrakis(triphenylphosphine)palladium(O) (0.011 g, 9.69 μmol). After 30 minutes at room temperature the crude reaction mixture was purified on silica gel (0-20% MeOH in DCM) to provide compound 41 (0.1249 g, 0.107 mmol, 55.3% yield). ESI-MS: calc. for C₆₉H₇₂N₁₁O₈⁺ (M+H₂O+H) 1182.6; found 1182.6.

### Part C:

Compound 42 (0.95 g, 3.13 mmol), K₂C₀₃ (0.801 g, 5.79 mmol), ACN (25 mL) and 3-bromoprop-1-ene (0.501 mL, 5.79 mmol) were stirred for 12 hours at room temperature. The crude reaction mixture was filtered through a Celite plug, washed with DCM and the filtrate was concentrated and purified on silica gel (0-100% EtOAc in Hexanes) to provide Boc-Glu(Y-OAllyl)-Ot-Bu (1.075 g, 94% yield). ESI-MS: calc. for C₁₇H₂₉NNaO₆⁺ (M+Na) 366.2; found 366.2.

To the intermediate Boc-Glu(γ-OAllyl)-Ot-Bu (1.075 g, 3.13 mmol) dissolved in DCM (15.65 mL), was added TFA (15.65 mL) and the reaction stirred for 12 hours at room temperature. The crude reaction mixture was concentrated to provide H-Glu(—OAllyl)-OH (0.586 g, 3.13 mmol, 100% yield). ESI-MS: calc. for C₈H₁₄NO₄⁺ (M+H) 188.1; found 188.1.

The intermediate H-Glu(—OAllyl)-OH (0.586 g, 3.13 mmol) was dissolved in water (15.65 mL) and acetone (15.65 mL), then NaHCO₃(0.789 g, 9.39 mmol) and allyl (2,5-dioxopyrrolidin-1-yl) carbonate (0.623 g, 3.13 mmol) were added and the reaction mixture was stirred for 12 hours at room temperature. The crude reaction mixture was concentrated, then acidified to pH 3 using 1N HCl, extracted with EtOAc, (3×) and the combined organic layers were washed with brine, dried over Na₂SO₄ and concentration to provided Alloc-Glu(γ-OAllyl)-OH (0.849 g, 3.13 mmol, 100% yield). ESI-MS: C₁₂H₁₇NNaO₆⁺ (M+Na) 294.1; found 294.1.

To the Alloc-Glu(γ—OAllyl)-OH intermediate (0.7 g, 2.58 mmol) was added H-Val-Ot-Bu (HCl salt) (0.541 g, 2.58 mmol), HOAt (0.369 g, 2.71 mmol), DMF (12.90 mL) and triethylamine (1.079 mL, 7.74 mmol). The resulting solution was stirred at 0° C. for 10 min, and then HATU (1.276 g, 3.35 mmol) was added and the reaction mixture was allowed to warm to room temperature and stirred for 12 hours. The crude reaction mixture was partitioned between DCM (100 mL) DCM and half-saturated NH₄Cl (100 mL). The aqueous layer was extracted with DCM, and the combined organic layers were washed with brine, dried over Na₂SO₄, and concentrated. The crude product was purified on silica gel (0-100% EtOAc in hexanes) to afford the intermediate compound 42-O-Bu (0.4942 g, 44.9% yield). ESI-MS: calc. for C₂₁H₃₅N₂O₇⁺ (M+H) 427.2; found 427.1.

To the intermediate compound 42-Ot-Bu (0.028 g, 0.065 mmol) was added DCM (1.3 mL) and TFA (0.247 mL, 3.25 mmol), and the reaction was stirred at room temperature for 3 hours. The reaction mixture was then concentrated to provide compound 43 (0.024 g, 100% yield). ESI-MS calc for C₇H₂₇N₂O₇⁺ (M+H) 371.2; found 371.2.

### Part D:

To compound 41 (0.0708 g, 0.061 mmol) was added HOAt (9.73 mg, 0.072 mmol), triethylamine (0.032 mL, 0.228 mmol), and a solution of compound 43 (0.024 g, 0.065 mmol) in DMF (1.300 mL). After stirring at room temperature for 5 minutes, HATU (0.030 g, 0.078 mmol) was added. The reaction was stirred for 12 hours at room temperature. The reaction mixture was diluted with deionized water (5 mL) and DCM (5 mL), the aqueous layer was extracted with DCM (2×). The combined organic layers were dried over Na₂SO₄ and concentrated. The crude product was purified on silica gel (0-15% MeOH in DCM) to provide compound 44 (0.074 g, 75% yield). ESI-MS calc for C₆H₉₄N₁₃O₁₃⁺ (M+H) 1516.7; found 1516.7.

### Part E:

To compound 44 (0.0739 g, 0.049 mmol) was added pyrrolidine (0.012 mL, 0.146 mmol), triphenylphosphine (3.19 mg, 0.012 mmol) and DCM (4.87 mL). To the stirred solution was added Pd(PPh₃)₄ (5.63 mg, 4.87 μmol), and the reaction was stirred for 1 hour at room temperature. The crude reaction mixture was concentrated and then suspended in DMF:H₂O (1:1, 3 mL). The suspension was centrifuged at 12 G for 14 minutes. The supernatant was filtered and then purified by RP-HPLC (10-90% ACN in H₂O with 0.1% v/v HOAc) to provide the deprotected intermediate (5.5 mg, 8.11% yield). ESI-MS calc. for C₇₉H₈₆N₁₃O₁₁⁺ (M+H) 1392.7; found 1392.7.

To the deprotected intermediate (5.5 mg, 3.95 μmol) were added DMF (0.7 mL), 2,5-dioxopyrrolidin-1-yl 3-(2-(2-(3-(2,5-dioxo-2,5-dihydro-1H-pyrrol-1-yl)propanamido)ethoxy)ethoxy)propanoate (5.04 mg, 0.012 mmol) and triethylamine (1.651 μL, 0.012 mmol). The reaction mixture was stirred for 1 hour at room temperature and then concentrated to provide Mtt protected compound 45 (6.73 mg, 100% yield). ESI-MS calc. for C₉₃H₁₀₄N₁₅O₁₇⁺ (M+H) 1702.8; found 1702.8.

To Mtt protected compound 44 (6.73 mg, 3.95 μmol) was added DCM (0.7 mL), 2,2,2-trifluoroethan-1-ol (200 μL, 2745 μmol) and HOAc (100 μL, 1748 μmol). The reaction mixture was stirred at room temperature for 2 hours, and then concentrated. The crude product was purified by RP-PLC (10-90% ACN in H₂O with 0.1% HCO2H) to provide compound 45 (2.5 mg, 43.8% yield). ESI-MS calc. for C₇₃H₈₈N₁₅O₁₇⁺ (M+H) 1446.7; found 1446.7.

### Part F:

Conjugate 46 was prepared from Trastuzumab and compound 45 as described in Example 2. The purified conjugate 46 had a PBD to trastuzumab ratio of 2.3 as determined by UV-Vis using molar extinction ε_{330 nm}==38,858.5 cm⁻¹ M⁻¹ and ϵ_{280 nm}=29,820.413 cm⁻¹ M⁻¹ for compound 9 and ε_{280 nm}=226,107 cm⁻¹ M⁻¹ for trastuzumab).

### Example 9: Synthesis of Trastuzumab Conjugate 57

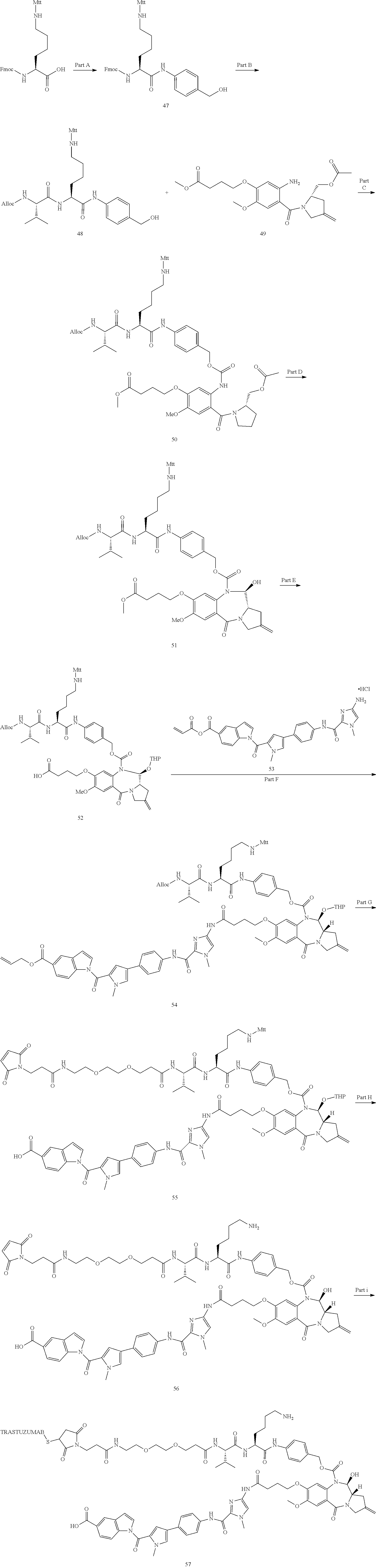

### Part A:

To a solution of Fmoc-Lys(Mtt)-OH (5 g, 8.00 mmol) in THF (25 mL) was added EEDQ (2.97 g, 12 mmol) followed by (4-aminophenyl)methanol (0.986 g, 8 mmol) and the mixture was stirred overnight, then concentrated and the residue was diluted with EtOAc (150 mL). The organic phase was washed with sat NaHCO₃. brine. dried over MgSO4 and concentrated. The crude product was purified on silica gel (0-70% EtOAc in hexane) to afford compound 47 (5.84 g, 100% yield) as a colorless foam. ESI MS calc for C₄₈H₄₈N₃O₄ (M+H) 730.4; found 730.3.

To a solution of compound 7 (4.8 g, 6.58 mmol) in ACN (30 mL) was added piperidine (3.25 ml, 32.9 mmol) After 45 min the mixture was diluted with ACN and filtered. The filtrate was concentrated, purified on a silica gel (0-10% MeOH in DCM) to afford the Fmoc-deprotected intermediate (1.05 g, 31.5% yield) as a colorless solid. ESI MS calc for C₃₃H₃₈N₃O₂ (M+H) 508.3; found 508.3.

To a solution of the Fmoc-deprotected intermediate (1.46 g, 2.88 mmol) in DMF (10 mL) was added Alloc-Val-OH (prepared as described in Example 6 except that L-Alanine was used instead of L-Valine, 0.579 g, 2.88 mmol) in DMF (˜1 mL) followed by EDC HCl (0.662 g, 3.45 mmol) and HOAt (0.470 g, 3.45 mmol). The mixture was stirred overnight at room temperature, concentrated, extracted with EtOAc (150 mL), washed with water (50 mL), sat NaHCO₃(50 mL), and brine (50 mL). The organic extracts were dried over MgSO₄, concentrated and purified on silica gel to afford compound 48 (1.38 g, 69.5% yield) as a colorless solid. ESI MS calc for C₄₂H₅₁N₄O₅ (M+H) 691.4; found 691.4.

### Part C:

To an ice-cold solution of compound 49 (1.14 g, 2.71 mmol, prepared as described in U.S. Ser. No. 15/630,068) in DCE (8 mL) was added saturated aqueous NaHCO₃(8 mL) under vigorous stirring. To this biphasic mixture was added a solution of triphosgene (0.483 g, 1.626 mmol) in DCE (˜2 mL). The mixture was stirred at room temperature for 1 h, then the aqueous layer was extracted with DCE (˜8 mL). The organic extracts were dried over MgSO₄ concentrated to ˜10 mL. The crude isocyanate solution was then slowly added over ˜15 min to a solution of compound 48 (1.38 g, 1.997 mmol), DMAP (0.272 g, 2.22 mmol), and TEA (0.378 mL) in DCE (˜10 mL) at ˜60° C. The mixture was stirred at 70° C., for 2 h, concentrated and purified on silica gel to afford compound 50 as a colorless foam (1.83 g, 59.4% yield).

### Part D:

To a mixture of compound 50 (1.741 g, 1.53 mmol) in MeOH (20 mL) and water (1 mL) was added K₂CO₃ (211 mg, 1.53 mmol). The mixture was stirred at room temperature for 45 min, concentrated, diluted with EtOAc and washed with water, brine, then dried over Na₂SO₄ and concentrated to yield a crude oil. This crude product was purified by chromatography (ISCO, 40 g column, 0-10% MeOH/DCM eluent) to afford the desired intermediate alcohol as a white foam (1.177 g, 70% yield). ESI MS calc for C₆₂H₇₅N₆O₁₂ (M+H) 1095.5; found 1095.5.

To a mixture of the intermediate alcohol (1.18 g, 1077 μmol) in DCM (12 mL) was added TEMPO (17 mg, 108 μmol) and BAIB (381 mg, 1.185 mmol). The mixture was stirred at room temperature under argon for 16 h, then additional TEMPO (9 mg, 54 μmol) and BAIB (190 mg, 592 μmol) were added. After 2 days the reaction mixture was concentrated then purified by chromatography (ISCO, 24 g column, 0-5% MeOH/DCM eluent), to yield compound 51 as a yellow foam (844 mg, 72% yield). ESI MS calc for C₆₂H₇₃N₆O₁₂ (M+H) 1093.5; found 1093.5.

### Part E:

To a solution of compound 51 (50 mg, 46 μmol) in THF (2 mL) was added pTsOH.H2O (2 mg) and DHP (200 μL). The mixture was stirred at room temperature for 5 h then additional pTsOH.H2O (14 mg) was added. After 7.5 h, the reaction mixture was diluted with EtOAc, then washed with saturated NaHCO₃ solution and brine. The organic extracts were dried over Na₂SO₄, concentrated to yield a light green oil. This crude material was then was purified (ISCO, 4 g column, 0-5% MeOH/DCM eluent), to afford the desired THP protected alcohol intermediate as a white powder (35 mg, 65% yield). ESI MS calc for C₆₇H₈₁N₆O₁₃ (M+H) 1177.6; found 1177.5.

To a solution of the THP protected alcohol intermediate (827 mg, 703 μmol) in dioxane (5.5 mL), water (1.7 mL) was added 1N NaOH (840 μL, 840 μmol) and then stirred at room temperature for 1 h. The reaction mixture was then diluted with water (80 mL) and the pH adjusted to 3 using 5% Citric acid solution with vigorous stirring. The aqueous layer was washed with EtOAc (2×) and the combined organic extracts were washed with brine (pH 3), dried over Na₂SO₄, and concentrated. The crude product was purified on silica gel (ISCO, 40 g column, O—10% MeOH/DCM eluent) to afford compound 52 as a white foam (540 mg, 66% yield). ESI MS calc for C₆₆H₇₉N₆O₁₃ (M+H) 1163.6; found 1163.5.

### Part F:

To a mixture of compound 52 (76 mg, 146 μmol) and compound 53 (174 mg, 146 μmol, prepared as described in US 15/639=0.968) in DMF (8.5 mL) was added HOAt (20 mg, 146 μmol) and TEA (41 μL, 730 μmol). The resulting mixture was stirred for 5 minutes, the HATU (72 mg, 190 μmol) was added under argon. After 17 h the reaction mixture was then concentrated, diluted with EtOAc and the organic phase was washed with water (3×) and brine. dried over Na₂SO₄, and concentrated to yield compound 54 as a sticky yellow foam (355 mg, 100% yield). ESI MS calc for C₉₅H₁₀₃N₁₂O₁₆ (M+H) 1667.8; found 1667.7.

### Part G:

To a solution of compound 54 (163 μmol) in DCM (18 mL) was added DABCO (55 mg, 489 μmol) and Pd(PPh₃)₄ (14 mg, 98 μmol) and the resulting mixture was stirred at room temperature for 0.5 h, then concentrated and purified by chromatography on silica gel (ISCO, 12 g column, 0-5% MeOH/DCM eluent) to afford the desired Alloc/allyl ester deprotected intermediate as a yellow amorphous solid (113 mg, 48% yield). ESI MS calc for C₈₈H₉₅N₁₂O₄ (M+H) 1543.7; found 1543.7.

To a solution of the Alloc/allyl ester deprotected intermediate (40 mg, 26 μmol) in DMF (1 mL) was added 2,5-dioxopyrrolidin-1-yl 3-(2-(2-(3-(2,5-dioxo-2,5-dihydro-1H-pyrrol-1-yl)propanamido)ethoxy)ethoxy)propanoate (12 mg, 29 μmol) and TEA (4.4 μL, 29 μmol) and the mixture was stirred under argon for 2 h. The reaction mixture was concentrated to afford crude compound 55 as a yellow oil (60 mg, 100% yield). ESI MS calc for C₁₀₂H₁₁₃N₁₄O₂₀ (M+H) 1853.8; found 1853.7.

### Part H:

Crude compound 55 (60 mg, 26 μmol) was dissolved in a mixture of HCOOH (800 μL), THF (100 μL) and H₂O (100 μL) and the mixture was stirred at room temperature for 7 h, concentrated and then purified by RP-HPLC (ISCO, 10-100% ACN/H₂O w/0.1% HCOOH eluent) to afford compound 56 as a white, puffy solid (5 mg, 3.3 μmol, 10% yield). ESI MS calc for C₇₇H₈₉N₁₄O₁₉ (M+H) 1513.6; found 1513.5.

### Part I:

Conjugate 57 was prepared from Trastuzumab and compound 56 as described in Example 3. The purified conjugate 57 had a PBD to trastuzumab ratio of 3.7 as determined by UV-Vis using molar extinction 330==31,180.8 cm⁻¹ M⁻¹ and ε_{280 nm}=24,632.8 cm⁻¹ M⁻¹ for compound 56 and ε_{280 nm}=226,107 cm⁻¹M⁻¹ for trastuzumab).

### Example 10: Synthesis of Trastuzumab Conjugate 60

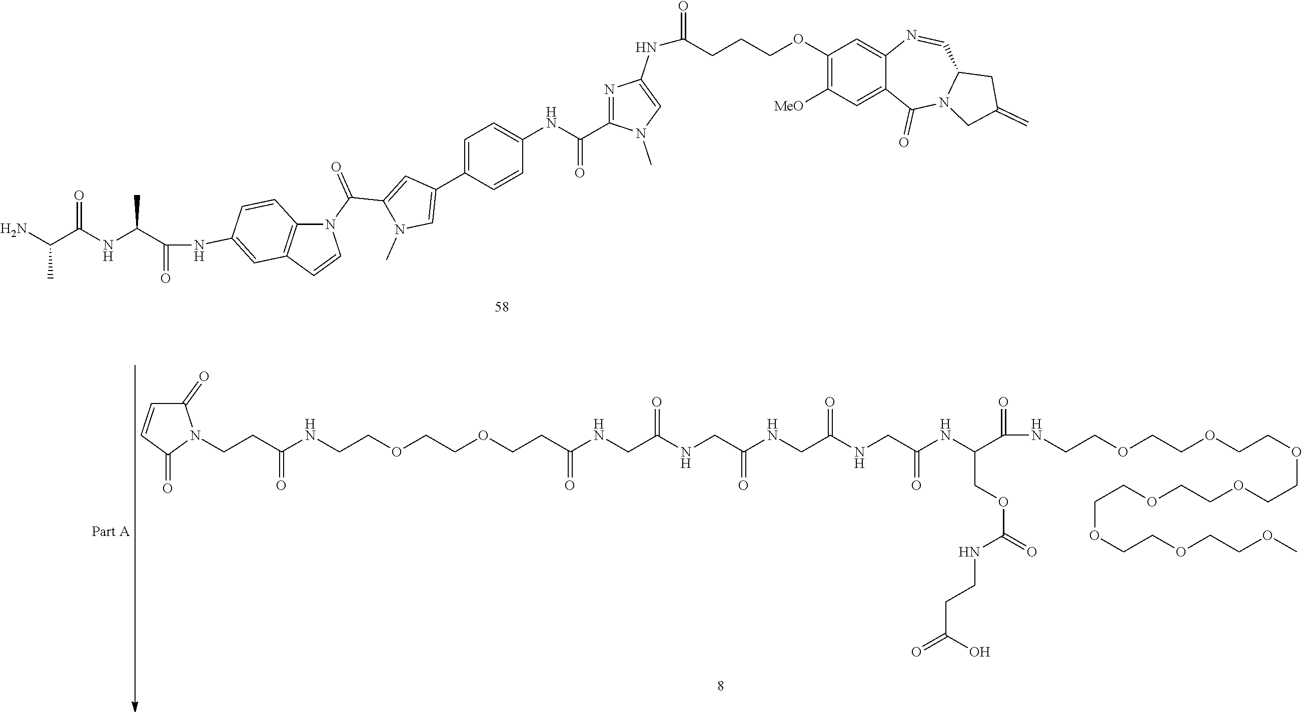 [SMILES: C=C1C[C@H]2C=Nc3cc(OCCCC(=O)Nc4cn(C)c(C(=O)Nc5ccc(-c6cc(C(=O)n7ccc8cc(NC(=O)[C@H](C)NC(=O)[C@H](C)N)ccc87)n(C)c6)cc5)n4)c(OC)cc3C(=O)N2C1 ; COCCOCCOCCOCCOCCOCCOCCOCCNC(=O)C(COC(=O)NCCC(=O)O)NC(=O)CNC(=O)CNC(=O)CNC(=O)CNC(=O)CCOCCOCCNC(=O)CCN1C(=O)C=CC1=O]

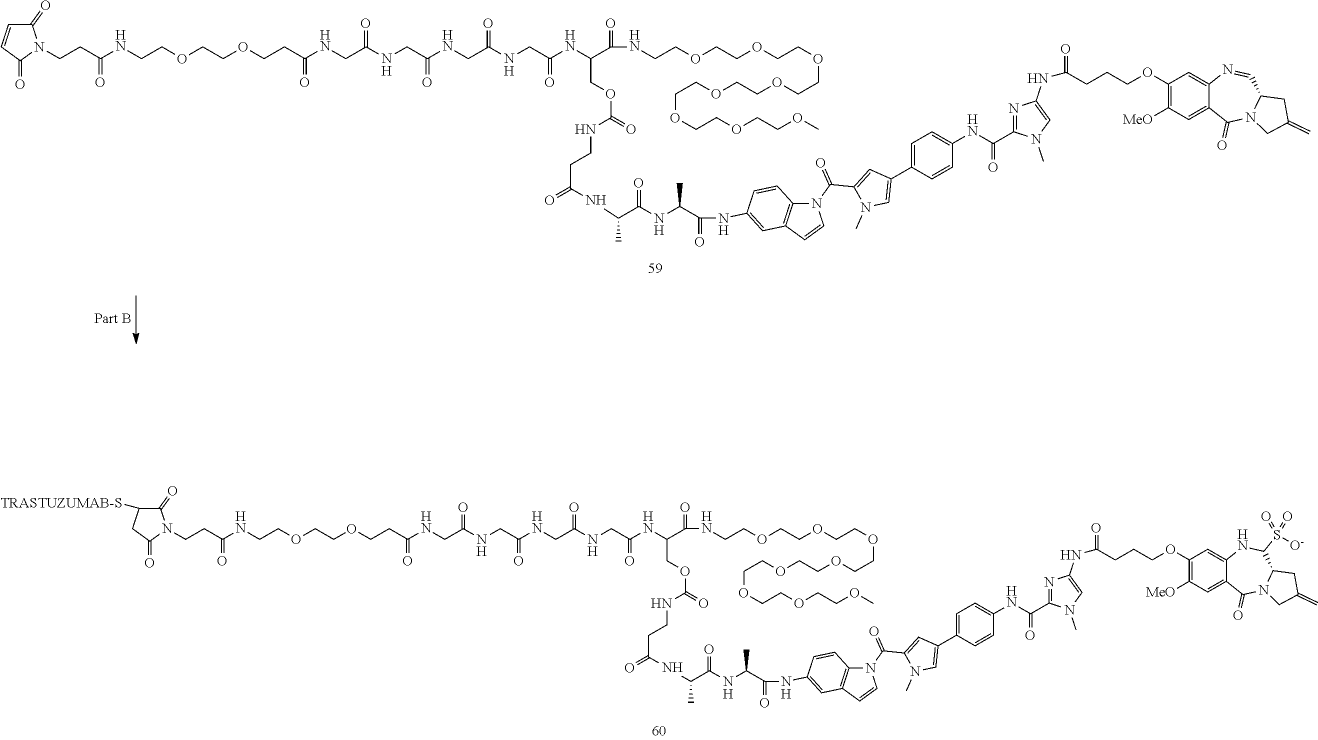

### Part A:

Compound 59 was prepared as described in Example 2 except that compound 58 was used instead of compound 7 to afford compound 59 as a pale yellow solid (47 mg, 50%). ESI-MS calc for C₉₅H₁₂₇N₂₀O₃₀ (M+2H) 1014.46; found 1014.37.

### Part B:

Conjugate 60 was prepared from Trastuzumab and compound 59 as described in Example 2 except that the crude conjugate was washed with a solution containing 20 mM MES, 0.25 mM NaHSO₃ and 0.1%/v/v Tween 80 (pH 5.8) prior to ion-exchange column purification. The purified conjugate 60 had a PBD to trastuzumab ratio of 4.3 as determined by UV-Vis using molar extinction ε_{330 nm}=38858.5 cm⁻¹ M⁻¹ and ε_{280 nm}=29820.413 cm⁻¹ M⁻¹ for compound 9 and ε_{280 nm}=226,107 cm⁻¹ M⁻¹ for trastuzumab).

### Example 11: Synthesis of Trop-2 Conjugate 61

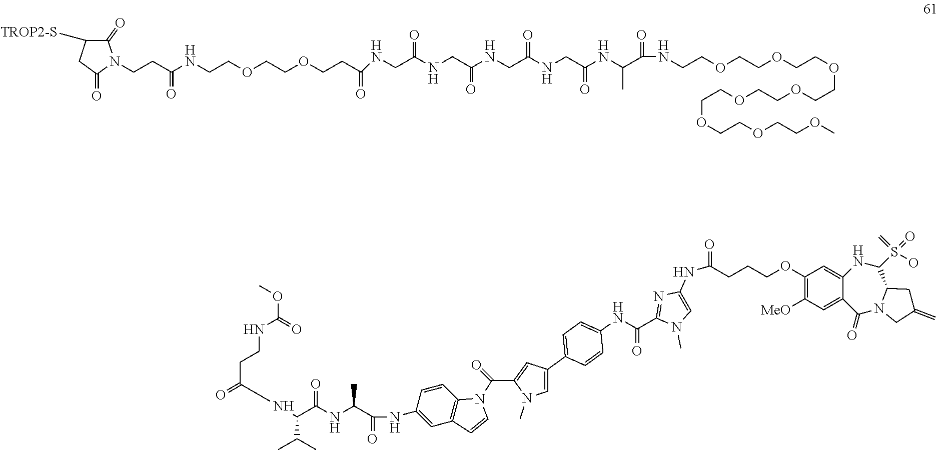

Conjugate 61 was prepared as described in Example 10 except that anti-Trop2 antibody was used instead of Trastuzumab. The purified conjugate 61 had a PBD to anti-Trop2 antibody ratio of 5.4 as determined by UV-Vis using molar extinction ε_{330 nm}=38858.5 cm⁻¹M⁻¹ and ε_{280 nm}=29820.413 cm⁻¹ M⁻¹ for compound 9 and ε_{280 nm}=226,372.2 cm⁻¹M⁻¹ for Anti-Trop2 antibody.

### Example 12: Synthesis of Rituximab Conjugate

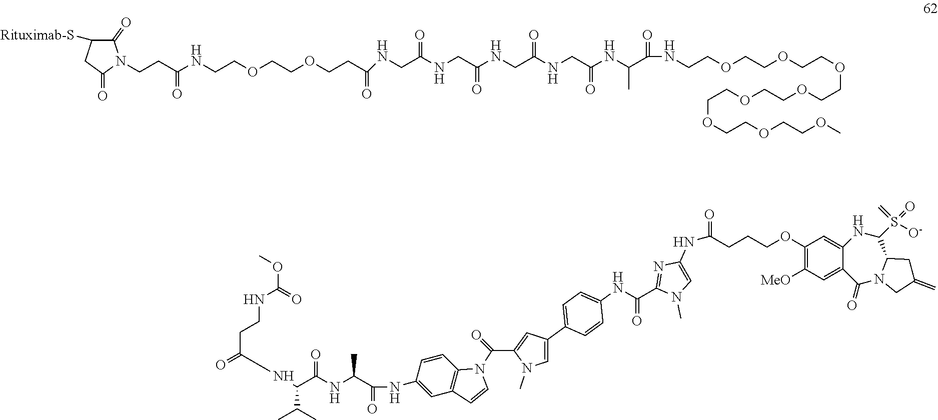

Conjugate 62 was prepared as described above in Example 10 except that Rituximab was used instead of Trastuzumab. The purified conjugate 62 had a PBD to Rituximab ratio of 5.5 as determined by UV-Vis using molar extinction ε_{330 nm}=38858.5 cm⁻¹ M⁻¹ and ε_{280 nm}=29820.413 cm⁻¹ M⁻¹ for compound 9 and ε_{280 nm}=228,263 cm⁻¹ M⁻¹ for Rituximab).

### Example 13: Synthesis of Trop-2 Conjugate 63

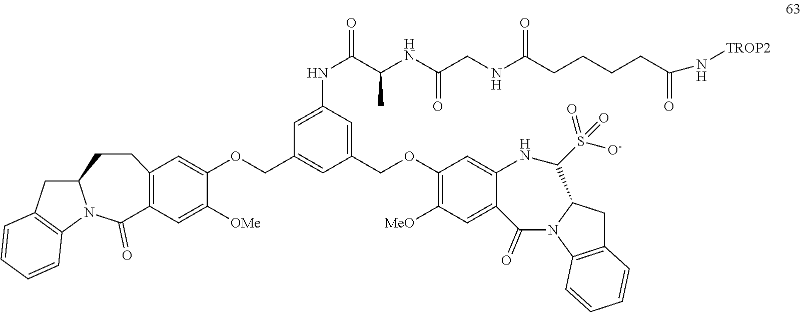

Conjugate 63 was prepared as described in US 2016/0082114A1 except that anti-Trop2 antibody. The purified conjugate 63 had a IGN to anti-Trop2 antibody ratio of 3.5 as determined by UV-Vis using molar extinction ε_{330 nm}=15,280 cm⁻¹ M⁻¹ and ε_{280 nm}=30,115 cm⁻¹ M⁻¹ for IGN (according to US2016/0082114A1) and E_{280 nm}=226,372.2 cm⁻¹ M⁻¹ for anti-Trop2 antibody).

### Example 13A: Synthesis of XMT-1535 Conjugate 63

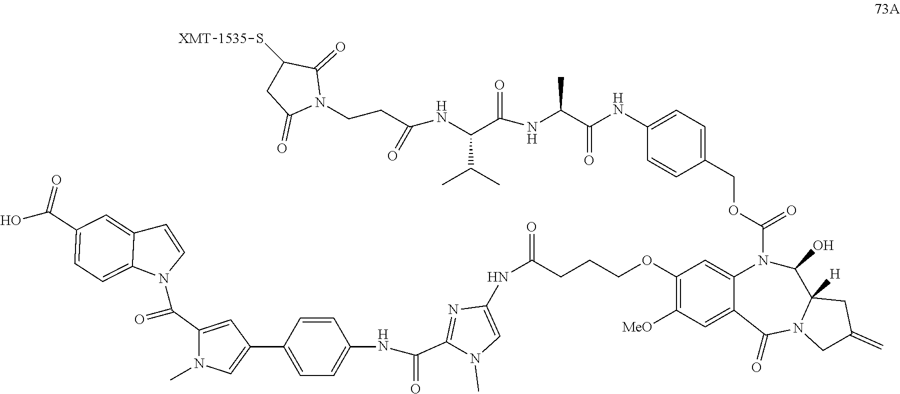

Conjugate 63A was prepared as described in Example 13 except that XMT-1535 antibody was used instead of Trastuzumab. The purified conjugate 63A had a PBD to XMT-1535 ratio of 2.5.

### Example 14: Synthesis of Rituximab Conjugate 64

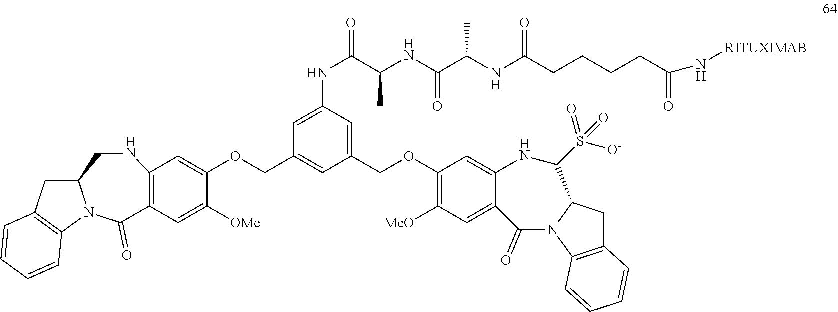

Conjugate 64 was prepared as described in US2016/0082114A1 except that Rituximab was used. The purified conjugate 64 had a IGN to Rituximab ratio of 1.7 as determined by UV-Vis using molar extinction ε_{330 nm}=15,280 cm⁻¹M⁻¹ and ε_{280 nm}=30,115 cm⁻¹M⁻¹ for IGN (according to patent US2016/0082114A1) and ε_{280 nm}=228,263 cm⁻¹ M⁻¹ for Rituximab).

### Example 14A: Synthesis of Rituximab Conjugate 64A

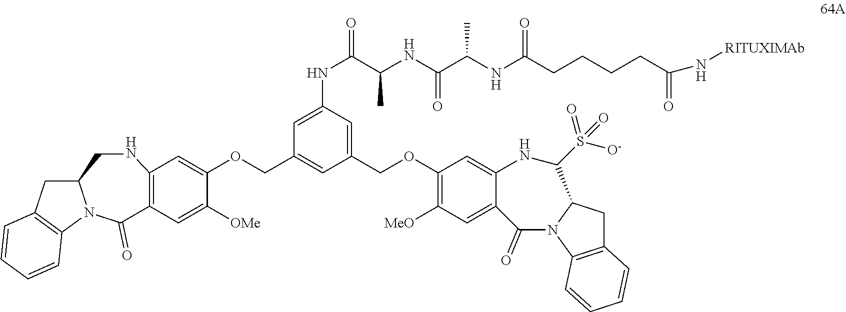

Conjugate 64 was prepared as described in Example 14. The purified conjugate 64A had a IGN to Rituximab ratio of 2.2 as determined by UV-Vis using molar extinction ε330 nm=15,280 cm⁻¹M⁻¹ and ε_{280 nm}=30,115 cm⁻¹ M⁻¹ for IGN (according to patent US2016/0082114A1) and ε_{280 nm}=228,263 cm⁻¹ M⁻¹ for Rituximab).

### Example 15: Synthesis of Trastuzumab Conjugate 67

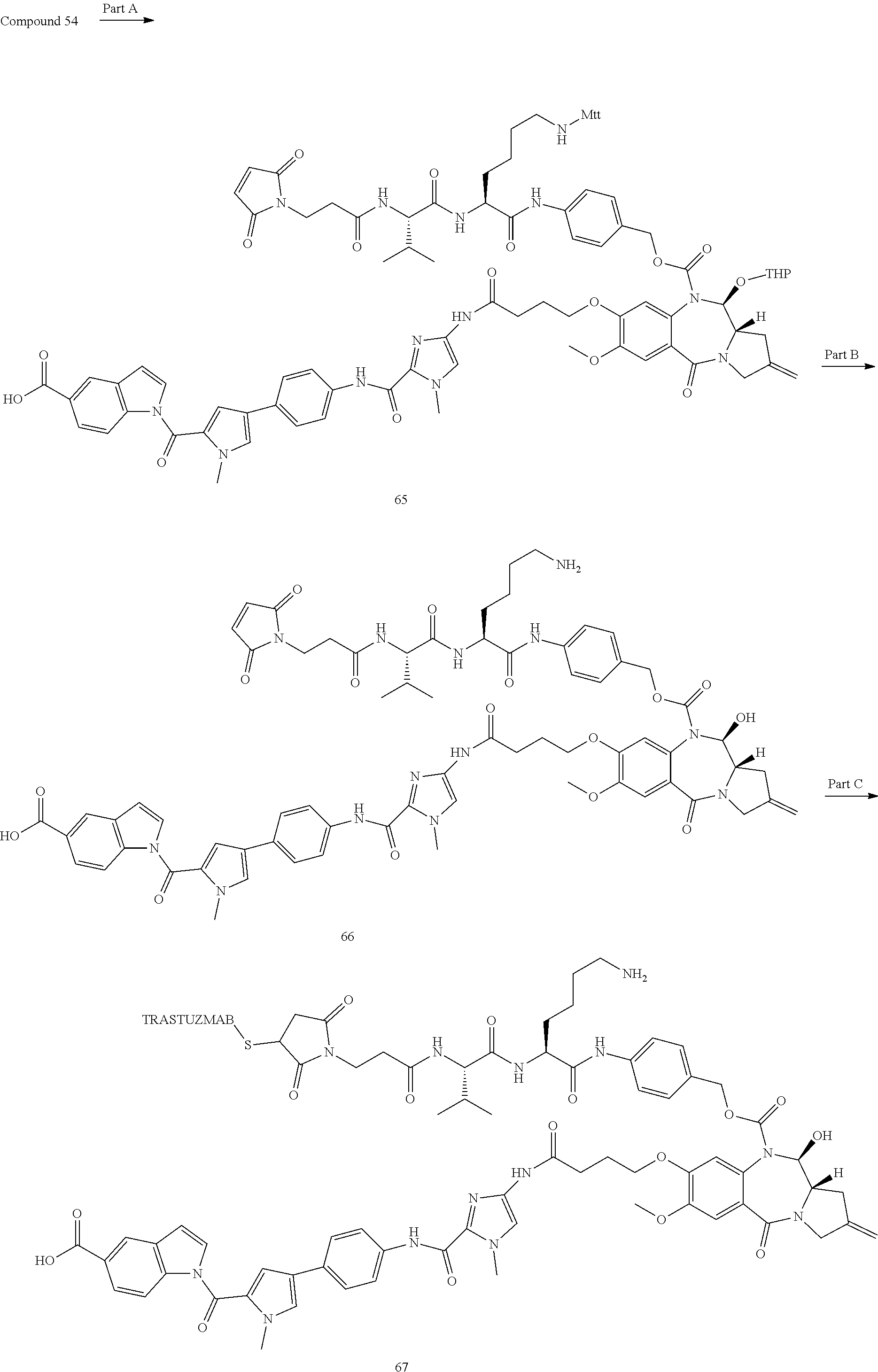

### Part A:

Compound 65 was prepared as described above in Example 9 except that 3-maleimidopropionic acid NHS ester was used instead of 2,5-dioxopyrrolidin-1-yl 3-(2-(2-(3-(2,5-dioxo-2,5-dihydro-1H-pyrrol-1-yl)propanamido)ethoxy)ethoxy)propanoate to afford crude compound 65 as a yellow oil. This material was used in the next step (Part B) without purification. ESI MS calc for C₉₅H₁₀₀N₃O₁₇ (M+H) 1694.7; found 1694.7.

### Part B:

Crude compound 65 (50 mg, 19 μmol) was treated with a mixture of HCOOH (800 μL), THF (100 μL) and H₂O (100 pL). After 3.5 h, the reaction mixture was concentrated, then purified by RP-HPLC (ISCO, 10-100% ACN/H2O w/0.1% HCOOH eluent) to afford compound 66 as an off-white, puffy solid (4 mg, 10% yield). ESI MS calc for C₇₀H₇₆N₁₃O₁₆ (M+H) 1354.6; found 1354.5.

### Part C:

Conjugate 67 was prepared from Trastuzumab and compound 66 as described in Example 3. The purified conjugate 67 had a PBD to trastuzumab ratio of 4.1 as determined by UV-Vis using molar extinction_{330 nm}=31,180.8 cm⁻¹ M⁻¹ and E_{280 nm}=24,632.8 cm⁻¹ M⁻¹ for compound 56 and E_{280 nm}=226,107 cm⁻¹ M⁻¹ for trastuzumab).

### Example 16: Synthesis of Trastuzumab Conjugate 71

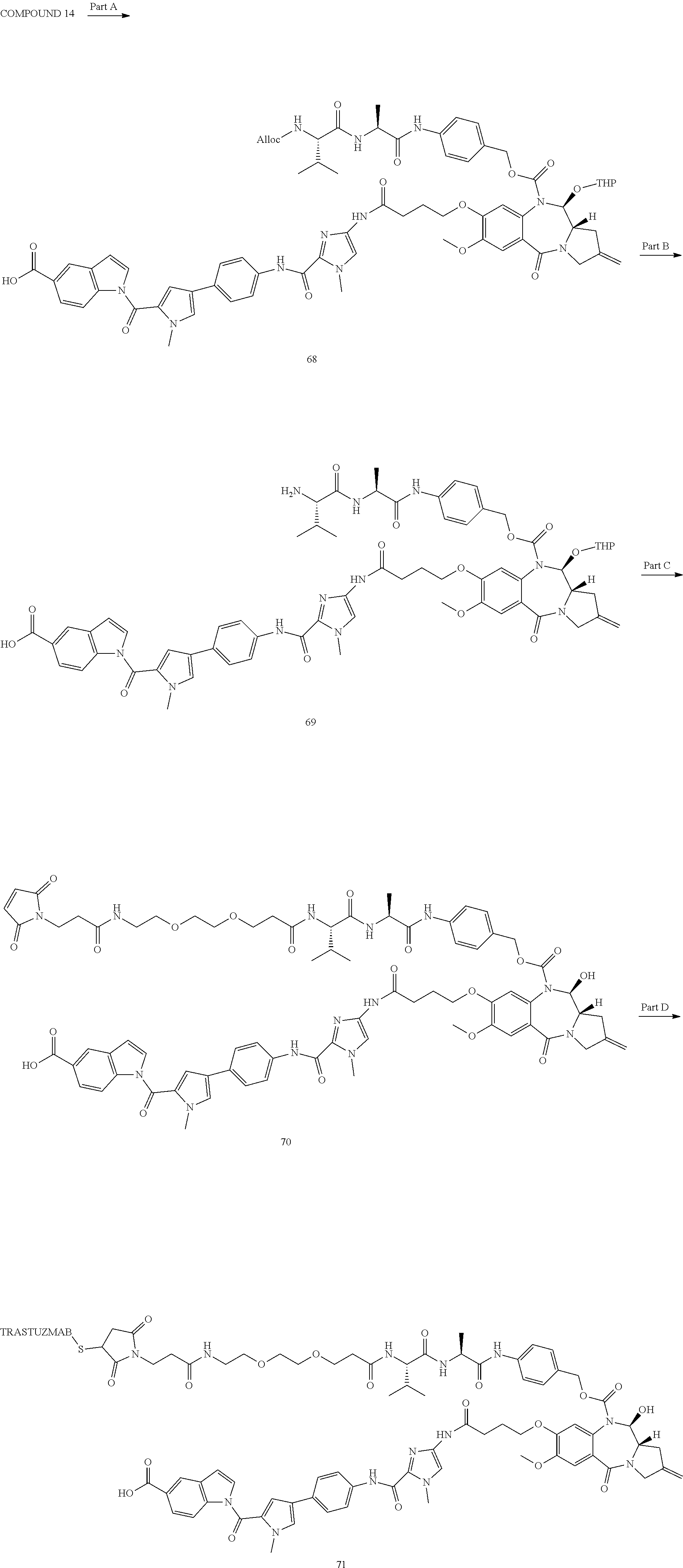

### Part A:

Compound 68 was prepared as described above in Example 9 except that compound 14 was used instead of compound 52 to afford crude compound 68 as a sticky yellow foam (186 mg, quant). The material was used in the next step (Part B) without purification. ESI MS calc for C₇₂H₈₀N₁₁O₁₆ (M+H) 1354.6; found 1354.5.

### Part B:

To a solution of crude compound 68 (186 mg) in DCM (2 mL) was added DABCO (4 eqs, 72 mg) and Pd(PPh₃)₄ (0.1 eqs, 18 mg). The mixture was stirred at room temperature for 30 mins, concentrated and purified by chromatography on silica gel (ISCO, 12 g column, 0-10% MeOH/DCM eluent) to afford compound 69 as a yellow amorphous solid (106 mg, 54% yield). ESI MS calc for C₆₁H₇₂N₁₁O₁₄ (M+H) 1230.5; found 1230.4.

### Part C:

To a solution of compound 69 (30 mg, 24 μmol) in DMF (1 mL) was added 2,5-dioxopyrrolidin-1-yl 3-(2-(2-(3-(2,5-dioxo-2,5-dihydro-1H-pyrrol-1-yl)propanamido)ethoxy)ethoxy)propanoate (12 mg, 26 μmol) and TEA (4 μL, 26 μmol). The mixture was stirred under argon for 2 h, concentrated to afford the crude maleimide intermediate as a yellow oil (40 mg, quant.). ESI MS calc for C₇₉H₉₀N₃O₂₀ (M+H) 1540.6; found 1540.5. This material was then dissolved in a mixture of HCOOH (960 μL), THF (160 μL) and water (160 μL) and stirred at room temperature for 1 h, concentrated and then purified by RP-HPLC (ISCO, 10-100% ACN/water w/0.1% HCOOH eluent) to afford compound 70 as a white, fluffy solid, (17 mg, 51% yield). ESI MS calc for C₇₄H₈₂N₁₃O₁₉ (M+H) 1456.6; found 1456.5.

### Part D:

Conjugate 71 was prepared from Trastuzumab and compound 70 as described in Example 3. The purified conjugate 71 had a PBD to trastuzumab ratio of 4.7 as determined by UV-Vis using molar extinction ε_{330 nm}=31,180.8 cm⁻¹ M⁻¹ and ε_{280 nm}=24,632.8 cm⁻¹ M⁻¹ or compound 56 and ε_{280 nm}=226,107 cm⁻¹M⁻¹ for trastuzumab).

### Example 17: Synthesis of Trastuzumab Conjugate 73

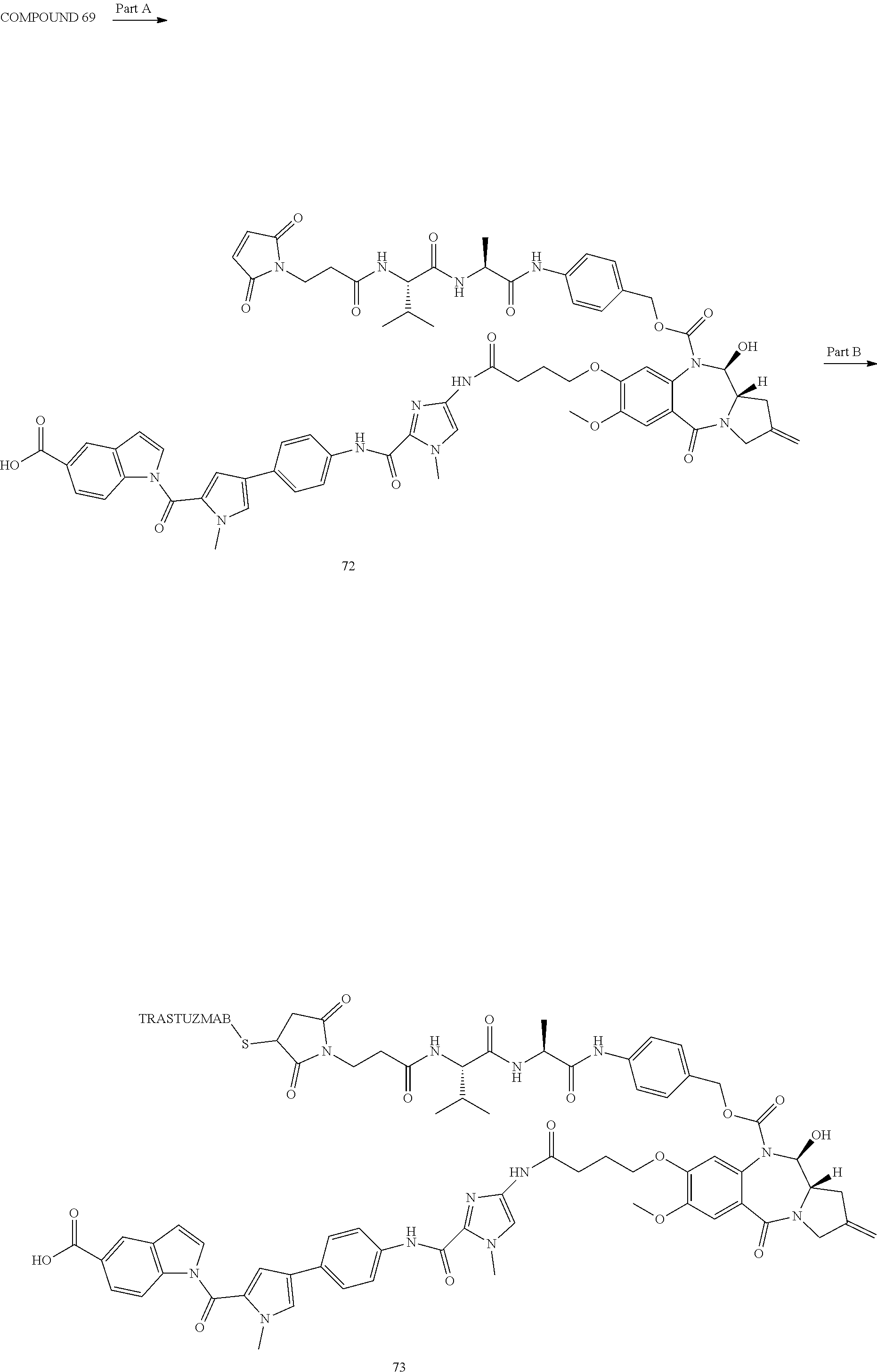

### Part A:

To a solution of compound 69 (40 mg, 33 μmol) in DMF (1 mL) was added 3-maleimido-propionic acid-NHS ester (1.1 eqs.10 mg) and TEA (1.1 eqs.5 pL). The mixture was stirred under argon for 2 h, concentrated to afford the crude maleimide intermediate as a yellow oil (50 mg, 100% yield). ESI MS calc for C₇₂H₇₇N₁₂O₁₇ (M+H) 1381.6; found 1381.5. This crude material (40 mg, 32 μmol) was dissolved in HCOOH (800 μL), THF (100 μL) and water (100 μL) was stirred at room temperature for 1.5 h, concentrated and then purified by RP-HPLC (ISCO, 4 g column, 10-100% ACN/water w/0.1% HCOOH eluent) to yield compound 72 as a yellow, fluffy solid (18 mg, 43% yield). ESI MS calc for C₆₇H₆₉N₂O₁₆ (M+H) 1297.5; found 1297.4.

### Part B:

Conjugate 73 was prepared from Trastuzumab and compound 72 as described in Example 3. The purified conjugate 73 had a PBD to trastuzumab ratio of 4.9 as determined by UV-Vis using molar extinction 6330 m=31,180.8 cm⁻¹ M⁻¹ and 6280 m=24,632.8 cm⁻¹M⁻¹ for compound 56 and ε_{280 nm}=226,107 cm⁻¹ M for trastuzumab).

### Example 17A: Synthesis of XMT-1535 Conjugate 73A

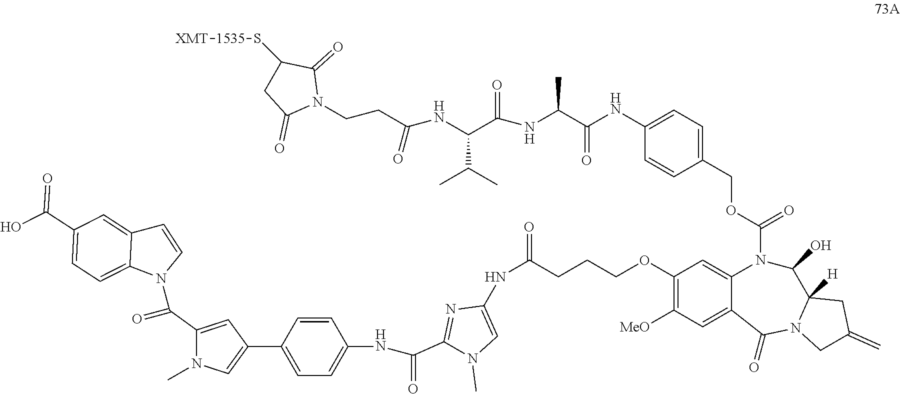

Conjugate 73A was prepared as described in Example 17 except that XMT-1535 antibody was used instead of Trastuzumab. The purified conjugate 73A had a PBD to XMT-1535 ratio of 3.5.

### Example 18: Synthesis of Trastuzumab Conjugate 79

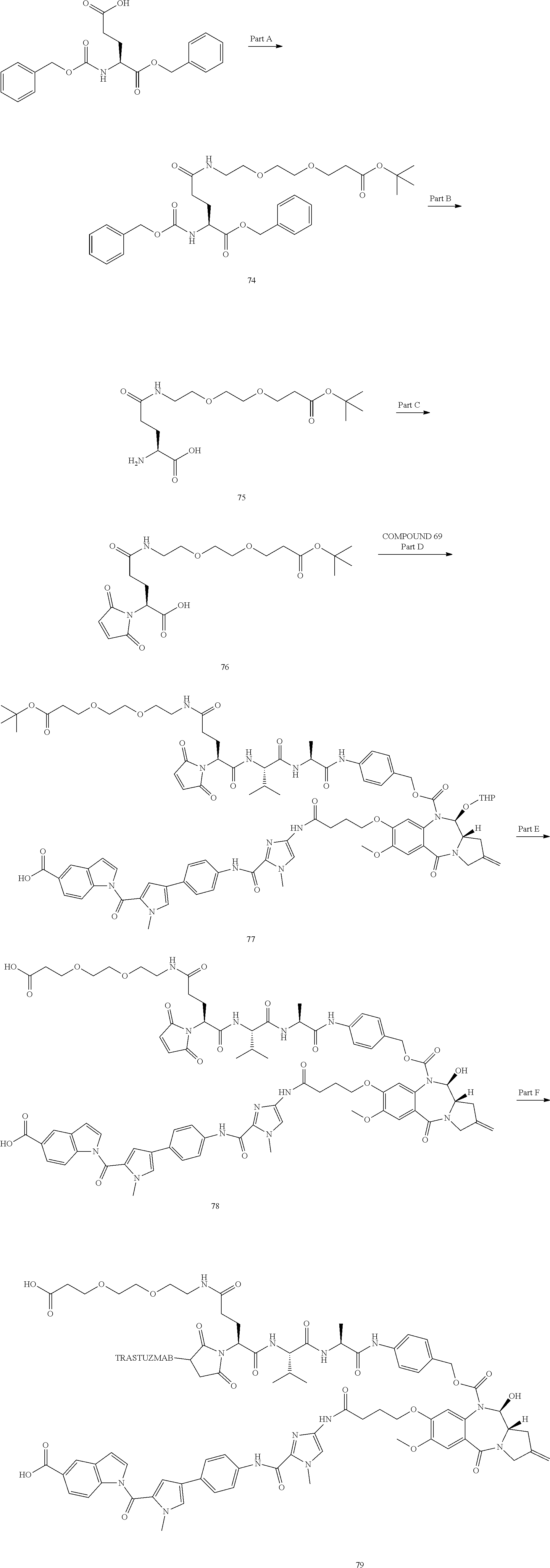

### Part A:

To a solution of Z-GIu-OBz (0.5 g, 1.356 mmol) in CH₂Cl₂ (3 mL) was added amino-DPEG2 t-butl ester (314 mg, 1.35 mmol), HATU (614 mg, 1.62 mmol), HOAt (220 mg, 1.62 mmol) and TEA (0.563 ml, 4.04 mmol). The reaction mixture was stirred overnight at room temperature, diluted with EtOAc, and washed with brine (3×). The organic phase was dried over Na₂SO₄, and concentrated. The crude product was purified on silica gel (ISCO, 40 g, 0-10% MeOH/DCM eluent) to afford compound 74 (390 mg, 49.4% yield). ESI MS: C₃H₄₂N₂O₉ (M+H) 587.3; found 587.3.

### Part B:

To a solution of compound 74 (385 mg., 0.656 mmol) in ethanol (5 ml) under nitrogen was added Pd—C(14 mg, 0.131 mmol). The reaction mixture was stirred under hydrogen overnight, filtered, washed with MeOH (3×) and concentrated to afford compound 75 as an oil (210 mg, 0.579 mmol, 88% yield). ESI MS: C₁₆H₃₀N₂O₇ (M+H) 363.2; found 363.2.

### Part C:

Compound 75(210 mg0.579 mmol) and maleic anhydride (56.8 mg0.579 mmol) in AcOH (3 ml) was stirred at room temperature overnight. The solution was concentrated, then diluted with toluene (7 mL), DMA (0.8 mL) and TEA (0.242 mL, 1.738 mmol), stirred for 2 days. The pH was adjusted to pH=1, the solution was concentrated and purified on silica gel (12 g, 0-20% MeOH/DCM eluent) to afford compound 76 (71 mg, 27.7% yield). ESI MS: C₂₀H₃₀N₂O₉ (M+H) 443.2; found 443.1.

### Part D:

To a solution of compound 69 (30 mg, 24 μmol) in DMF (1 mL) was added compound 76 (1.1 eqs, 11 mg), HOAt (1 eqs, 3.3 mg), and TEA (3.0 eqs, 10 μL). The mixture was stirred for 5 minutes before HATU (1.3 eqs, 12 mg) was added and the reaction was stirred at room temperature for 21 h, concentrated to afford crude compound 77 as a yellow, amorphous solid (40 mg, 100% yield). This material was used in the next step without further purification. ESI MS calc for (M+H) 1655.8; found 1655.6.

### Part E:

To a solution of crude compound 77 (42 mg, 23 μmol) in DCM (850 μL) was added TFA (150 μL) and stirred at room temperature for 1.5 h. The reaction mixture was concentrated and then purified by RP-HPLC (ISCO, 4 g column, 10-100% ACN/water w/0.1% HCOOH eluent), to afford compound 78 as a white, fluffy solid (2 mg, 6% yield). ESI MS calc for C₇₆H₁N₁₃₀₂₁ (M+H) 1514.6; found 1514.5.

### Part F:

Conjugate 79 was prepared from Trastuzumab and compound 78 as described in Example 3. The purified conjugate 79 had a PBD to trastuzumab ratio of 2.5 as determined by UV-Vis using molar extinction ε_{330 nm}=31,180.8 cm⁻¹ M⁻¹ and ε_{280 nm}=24,632.8 cm⁻¹ M⁻¹ for compound 56 and ε_{280 nm}=226,107 cm⁻¹ M⁻¹ for trastuzumab).

### Example 19: Synthesis of Trastuzumab Conjugate 86

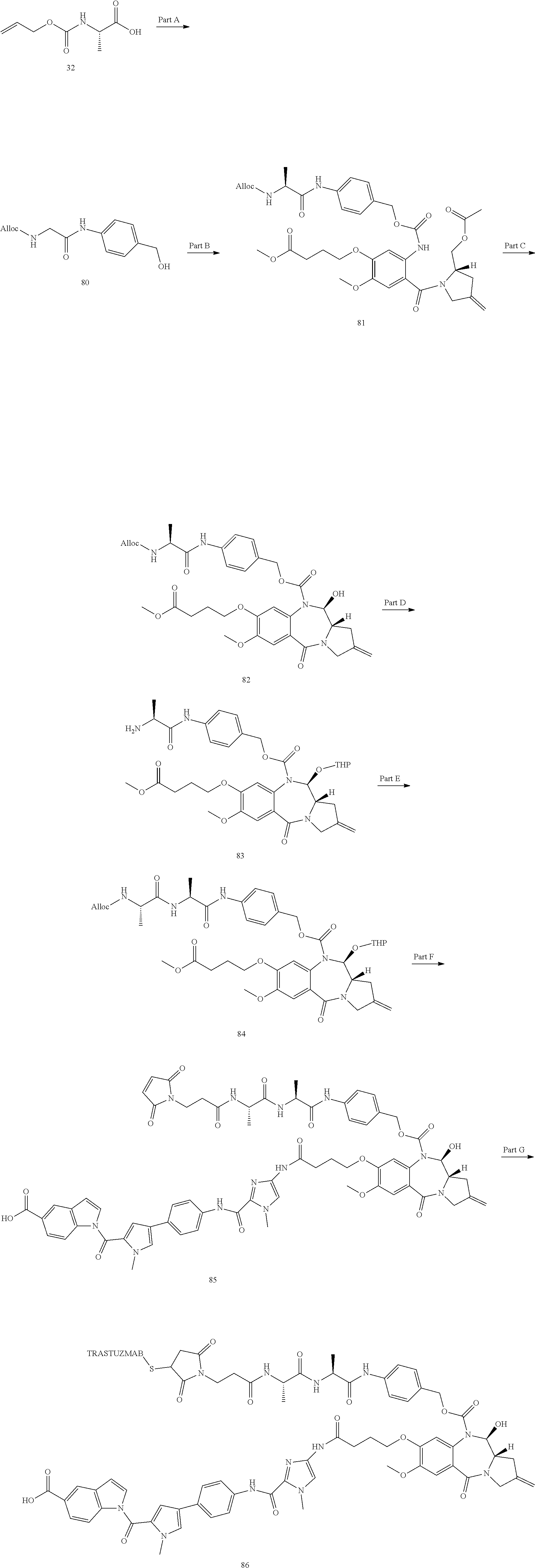

### Part A:

Compound 80 was prepared as described in Example 9 except that compound 32 was used instead of Fmoc-Lys(Mtt)-OH to afford compound 80 (93% yield.). ESI-MS calc for C₁₄H₁₉N₂O₄ (M+H) 279.1; found 279.1.

### Part B:

Compound 81 was prepared as described in Example 9 for the synthesis of compound 49 except compound 80 was used instead of compound 47 to afford compound 81 (62% yield.). ESI-MS calc for C₃₆H₄₅N₄O₁₂ (M+H) 725.3; found 725.3.

### Part C:

Compound 82 was prepared as described in Example 9 for the synthesis of compound 50 except compound 81 was used instead of compound 49 to afford compound 82 (76% yield, for 2 steps). ESI-MS calc for C₃₄H₄₁N₄O₁₁ (M+H) 681.3; found 681.3.

### Part D:

The THP ether of compound 82 was prepared as described in Example 3 for the synthesis of 13 except that compound 82 was used instead of compound 12 to afford the THP protected intermediate. ESI-MS calc for C₃₉H₄₉N₄O₁₂ (M+H) 765.3; found 765.3. To the THP protected intermediate (0.886 g, 1.158 mmol) was added pyrrolidine (0.285 ml, 3.47 mmol), triphenylphosphine (0.076 g, 0.290 mmol), and DCM (11.58 ml), followed by the addition of Pd(PPh₃)₄ (0.067 g, 0.058 mmol), and the reaction mixture was stirred at room temperature for 30 minutes, then purified on silica gel (0-25% MeOH in DCM) to afford compound 83 (0.782 g, 99% yield). ESI-MS calc for C₃₅H₄₅N₄O₁₀ (M+H) 681.3; found 681.2.

### Part E:

To compound 83 (0.782 g, 1.149 mmol) was added HOAt (0.156 g, 1.149 mmol), compound 31 (0.219 g, 1.264 mmol), DMF (11.49 ml) and DIEA (0.700 ml, 4.02 mmol). To this solution was added HATU (0.524 g, 1.378 mmol). The reaction mixture was stirred at room temperature for 12 hours, concentrated, and purified on silica gel (0-10% MeOH in DCM) to afford compound 84 (0.731 g, 0.874 mmol, 76% yield). ESI-MS calc for C₄₂H₅₄N₅O₁₃ (M+H) 836.4; found 836.3.

### Part F:

Compound 84 was reacted as described in Example 9 except that 3-maleimidopropionic acid-NHS ester was used instead of 2,5-dioxopyrrolidin-1-yl 3-(2-(2-(3-(2,5-dioxo-2,5-dihydro-1H-pyrrol-1-yl)propanamido)ethoxy)ethoxy)propanoate to afford compound 85. ESI-MS calc for C₆₅H₆₅N₁₂₀₁₆ (M+H) 1269.5; found 1269.4.

### Part G:

Conjugate 86 was prepared from Trastuzumab and compound 85 as described in Example 3. The purified conjugate 86 had a PBD to trastuzumab ratio of 4.6 as determined by UV-Vis using molar extinction ε_{330 nm}==31,180.8 cm⁻¹ M⁻¹ and ε_{280 nm}=24,632.8 cm⁻¹ M⁻¹ for compound 56 and ε_{280 nm}=226,107 cm⁻¹ M⁻¹ for trastuzumab).

### Example 20: Synthesis of Trastuzumab Conjugate 94

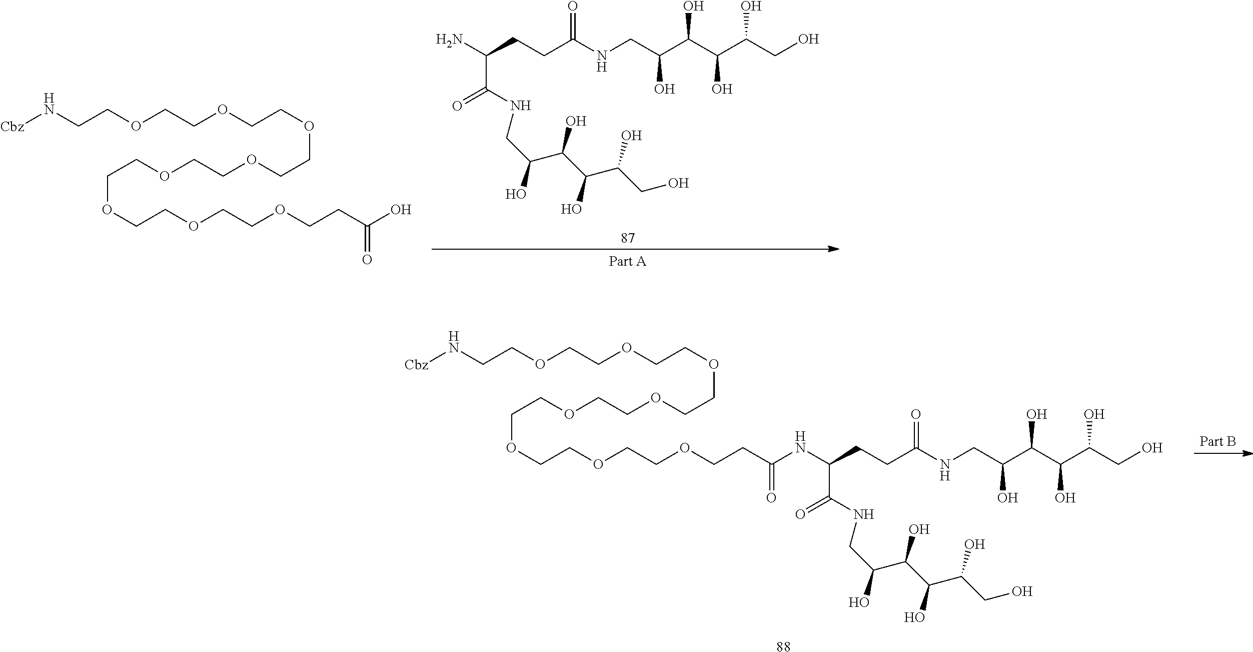 [SMILES: O=C(O)CCOCCOCCOCCOCCOCCOCCOCCOCCNC(=O)OCc1ccccc1 ; N[C@@H](CCC(=O)NC[C@H](O)[C@@H](O)[C@H](O)[C@H](O)CO)C(=O)NC[C@H](O)[C@@H](O)[C@H](O)[C@H](O)CO ; "87" ; O=C(CC[C@H](NC(=O)CCOCCOCCOCCOCCOCCOCCOCCOCCNC(=O)OCc1ccccc1)C(=O)NC[C@H](O)[C@@H](O)[C@H](O)[C@H](O)CO)NC[C@H](O)[C@@H](O)[C@H](O)[C@H](O)CO ; "88"]

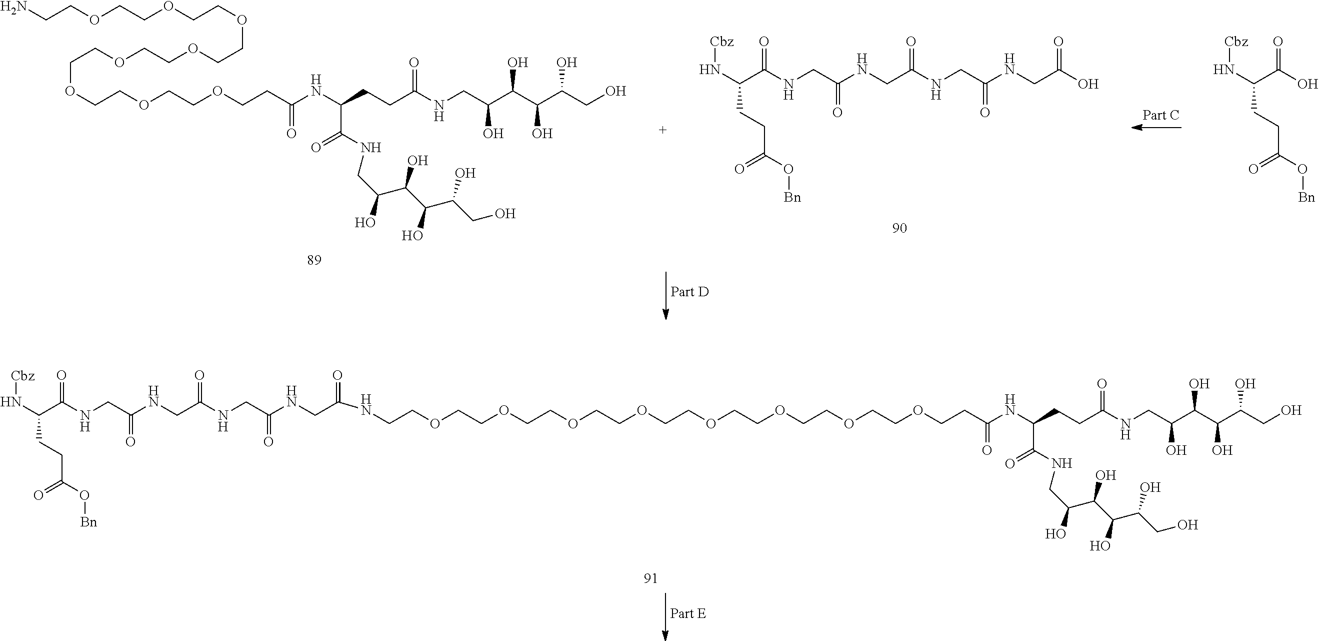

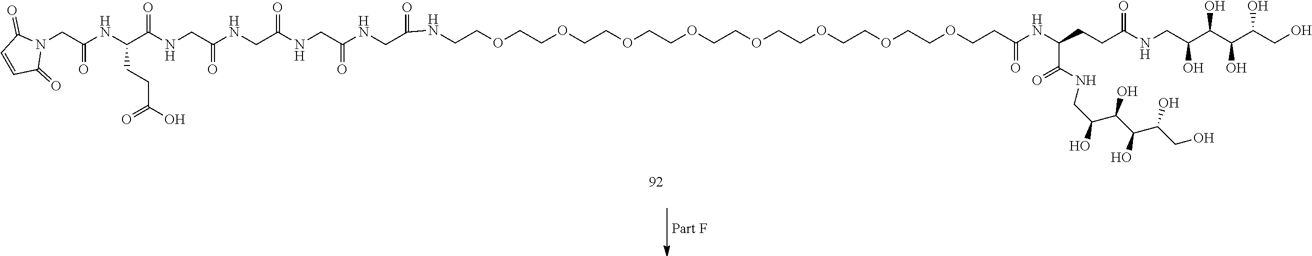 [SMILES: O=C(O)CC[C@H](NC(=O)CN1C(=O)C=CC1=O)C(=O)NCC(=O)NCC(=O)NCC(=O)NCC(=O)NCCOCCOCCOCCOCCOCCOCCOCCOCCC(=O)N[C@@H](CCC(=O)NC[C@H](O)[C@@H](O)[C@H](O)[C@H](O)CO)C(=O)NC[C@H](O)[C@@H](O)[C@H](O)[C@H](O)CO ; "92"]

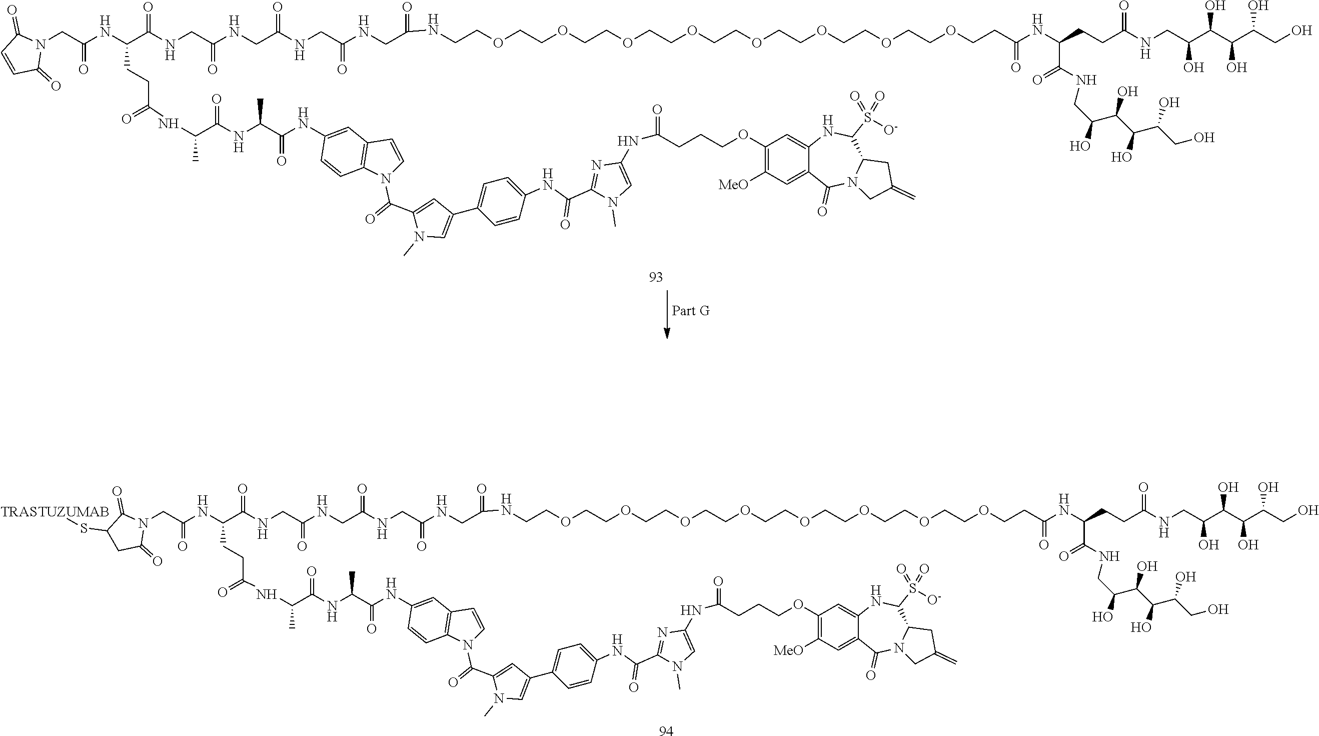

Part A:

Cbz-PEG8-CO₂H (900 mg, 1.56 mmol) and compound 87 (814 mg, 1.72 mmol) were dissolved in DMF (16 mL). To this mixture was added HOBt (47.9 mg, 0.31 mmol) and EDCI (330 mg, 1.72 mmol) in one portion and the mixture was stirred overnight at room temperature. The reaction mixture was concentrated and purified by RP-HPLC (ISCO, 275 g column, 0-40% ACN/water w/0.1% HCOOH eluent) to afford compound 88 (850 mg, 53% yield) as an off-white solid. ESI MS calc for C₄₄H₇₉N₄O₂₃ (M+H) 1031.5; found 1031.5.

### Part B:

To compound 88 (700 mg, 0.68 mmol) in ethanol/water (10:1, 68 mL) was added 10% palladium on carbon (181 mg, 0.17 mmol). The mixture was stirred under hydrogen at 30 psi in a Parr bomb. After 6 h, the reaction was filtered through a Celite pad, washed with EtOH/water (3:1, 3×), concentrated to afford compound 89 as a colorless oil which was used in the next step without further purification. ESI MS calc for C₃₆H₇₃N₄O₂₁ (M+H) 897.5; found 897.4.

### Part C:

(S)-5-(benzyloxy)-2-(((benzyloxy)carbonyl)amino)-5-oxopentanoic acid (2.0 g, 5.39 mmol) and 1-hydroxypyrrolidine-2,5-dione (0.74 g, 6.46 mmol) in DCM (50 mL) and DMF (5 mL) was cooled in an ice/water bath. Then DMAP (0.789 g, 6.46 mmol) and N,N′-diisopropylcarbodiimide (1.00 ml, 6.46 mmol) were added sequentially and the mixture was allowed to warm to room temperature. After 1 h, the DCM was removed via rotary evaporation. To the resulting DMF solution, a solution of tetraglycine (0.53 g, 2.14 mmol) in acetonitrile (20 mL) and water (20 mL) was added followed by sodium bicarbonate (0.18 g, 2.14 mmol). The reaction was stirred at room temperature for 18 h, concentrated, filtered and the filtrate was purified via RP-HPLC (ISCO, 150 g column, 0-50% ACN/water w/0.1% HCOOH eluent) to afford compound 90 (680 mg, 21% yield) of as a white fluffy solid. ESI MS calc for C₂₈H₃₄N₅O₁₀ (M+H) 600.2; found 600.2.

### Part D:

To compound 89 (598 mg, 0.68 mmol) in DMF (11 mL) was added compound 90 (400 mg, 0.67 mmol), followed by HOBt (20 mg, 0.13 mmol) and EDC (141 mg, 0.73 mmol). The reaction was stirred at room temperature for 18 h, concentrated and purified by RP-HPLC (ISCO, 100 g column, 0-50% ACN/water w/0.1% HCOOH eluent) to afford compound 91 (230 mg, 23% yield) of as a white fluffy solid. ESI MS calc for C₆₄H₁₀₄N₉O₃₀ (M+H) 1478.7; found 1478.6.

### Part E:

To compound 91 (230 mg, 0.15 mmol) in ethanol/water (10:1, 15 mL) was added 10% palladium on carbon (41 mg, 0.04 mmol). The mixture was stirred under hydrogen at 30 psi in a Parr bomb. After 18 h, the reaction was filtered through a Celite pad, washed with EtOH/water (3:1, 3×), concentrated, then dissolved in DMF (4 mL) and cooled in an ice/water bath. Triethylamine (0.021 ml, 0.15 mmol) and 2,5-dioxopyrrolidin-1-yl 2-(2,5-dioxo-2,5-dihydro-1H-pyrrol-1-yl)acetate (38.2 mg, 0.15 mmol) were added sequentially and the reaction mixture was allowed to warm to room temperature. After 30 minutes, an aliquot (25% by volume) was used directly in the next step. The remaining portion was purified by RP-HPLC (ISCO, 100 g column, O—40% ACN/water w/0.1% HCOOH eluent) to afford compound 92 (120 mg, 58% yield, 2 steps) as a white solid. ESI MS calc for C₂₈H₃₄N₅O₁₀ (M+H) 1391.6; found 1391.5.

### Part F:

To crude compound 92 (36 mg, 0.026 mmol) in DMF (1 mL) was added HATU (10 mg, 0.026 mmol), HOAt (4 mg, 0.026 mmol) and DIEA (5.68 μl, 0.033 mmol). The reaction mixture was stirred for 15 min at room temperature then stirring for an additional 5 minutes in an ice/water bath. Compound 57 (20 mg, 0.022 mmol) was added and the reaction was allowed to warm to room temperature. The reaction mixture was diluted with an equal amount of HOAc (0.1% in water), then purified by RP-HPLC (ISCO, 100 g column, 0-60% ACN/water w/0.1% HCOOH eluent) to afford compound 93 (5 mg, 10% yield) as a white solid. ESI MS calc for C₁₀₄H₁₄₄N₂₁O₃₈ (M+H) 2295.0; found 2295.8.

### Part G:

Conjugate 94 was prepared from Trastuzumab and compound 93 as described in Example 10. The purified conjugate 80 had a PBD to trastuzumab ratio of 4.4 as determined by UV-Vis using molar extinction ε_{330 nm}=38858.5 cm⁻¹ M⁻¹ and ε_{280 nm}=29820.413 cm⁻¹ M⁻¹ for compound 9 and ε_{280 nm}=226,107 cm⁻¹ M⁻¹ for trastuzumab).

### Example 20A: Synthesis of XMT-1535 Conjugate 94A

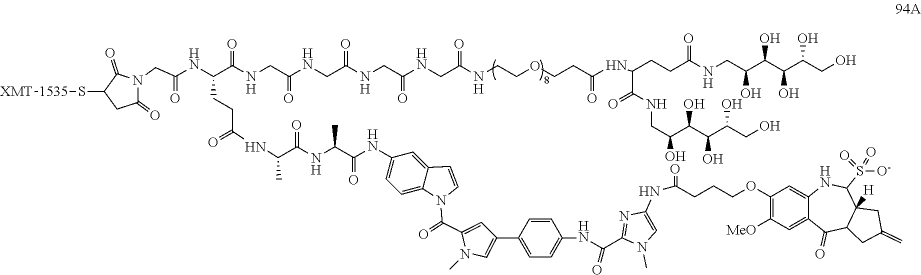

Conjugate 94A was prepared as described in Example 20 except that XMT-1535 antibody was used instead of Trastuzumab. The purified conjugate 94A had a PBD to XMT-1535 ratio of 4.1.

### Example 20B: Synthesis of Rituximab Conjugate 94B

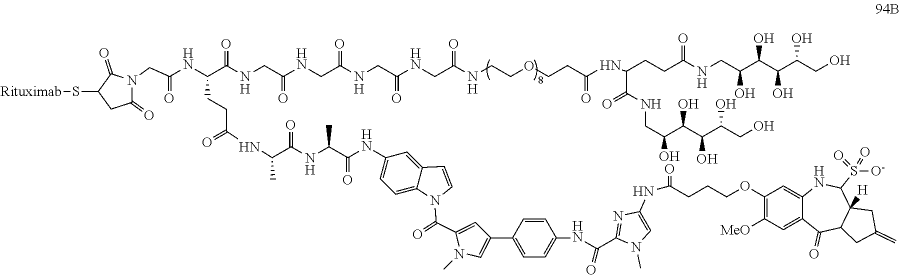

Conjugate 94B was prepared as described above in Example 20 except that Rituximab was used instead of Trastuzumab. The purified conjugate 94B had a PBD to Rituximab ratio of 4.6 as determined by UV-Vis using molar extinction ε_{330 nm}=38858.5 cm⁻¹ M⁻¹ and ε_{280 nm}=29820.413 cm⁻¹M⁻¹ for compound 9 and ε_{280 nm}=228,263 cm⁻¹ M⁻¹ for Rituximab).

### Example 21: Synthesis of Trastuzumab Conjugate 105

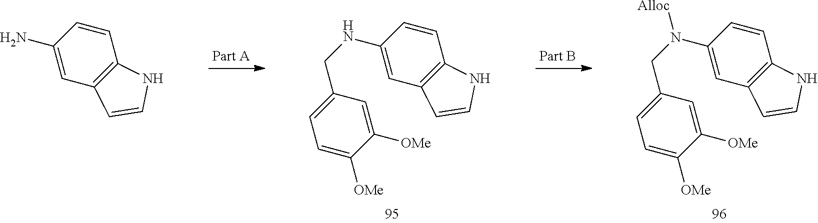

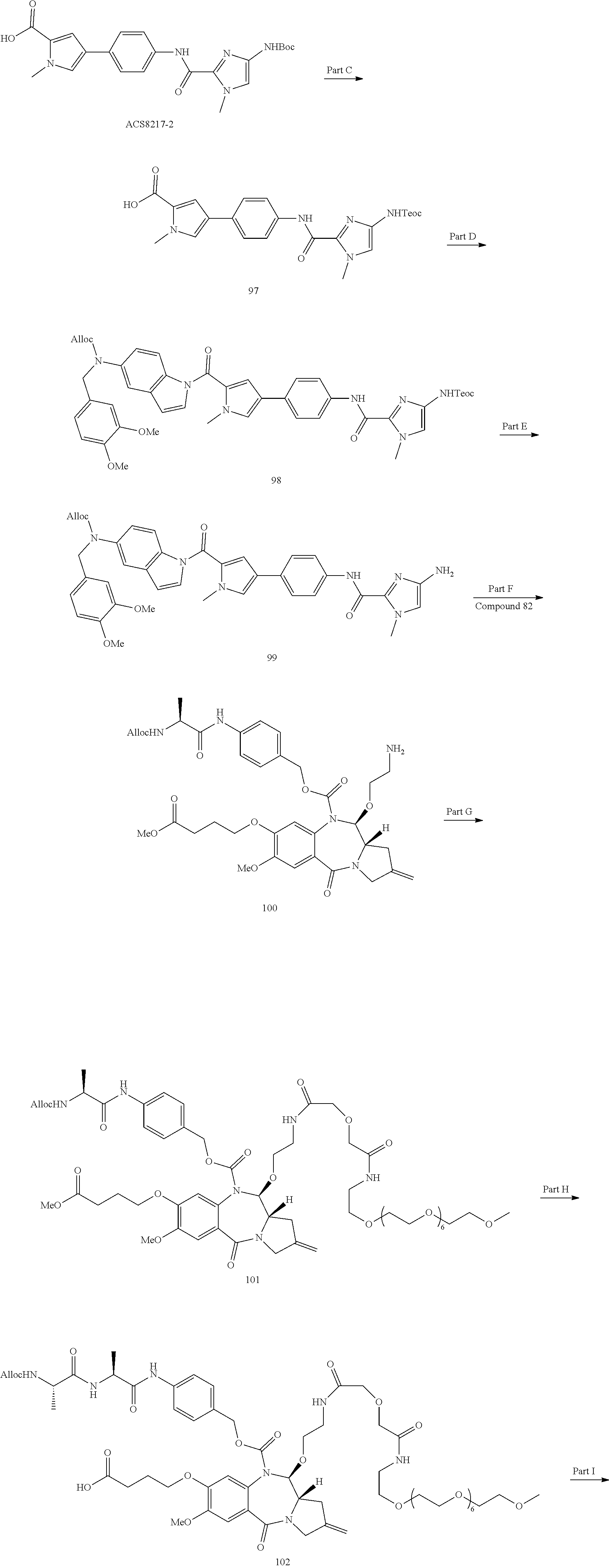

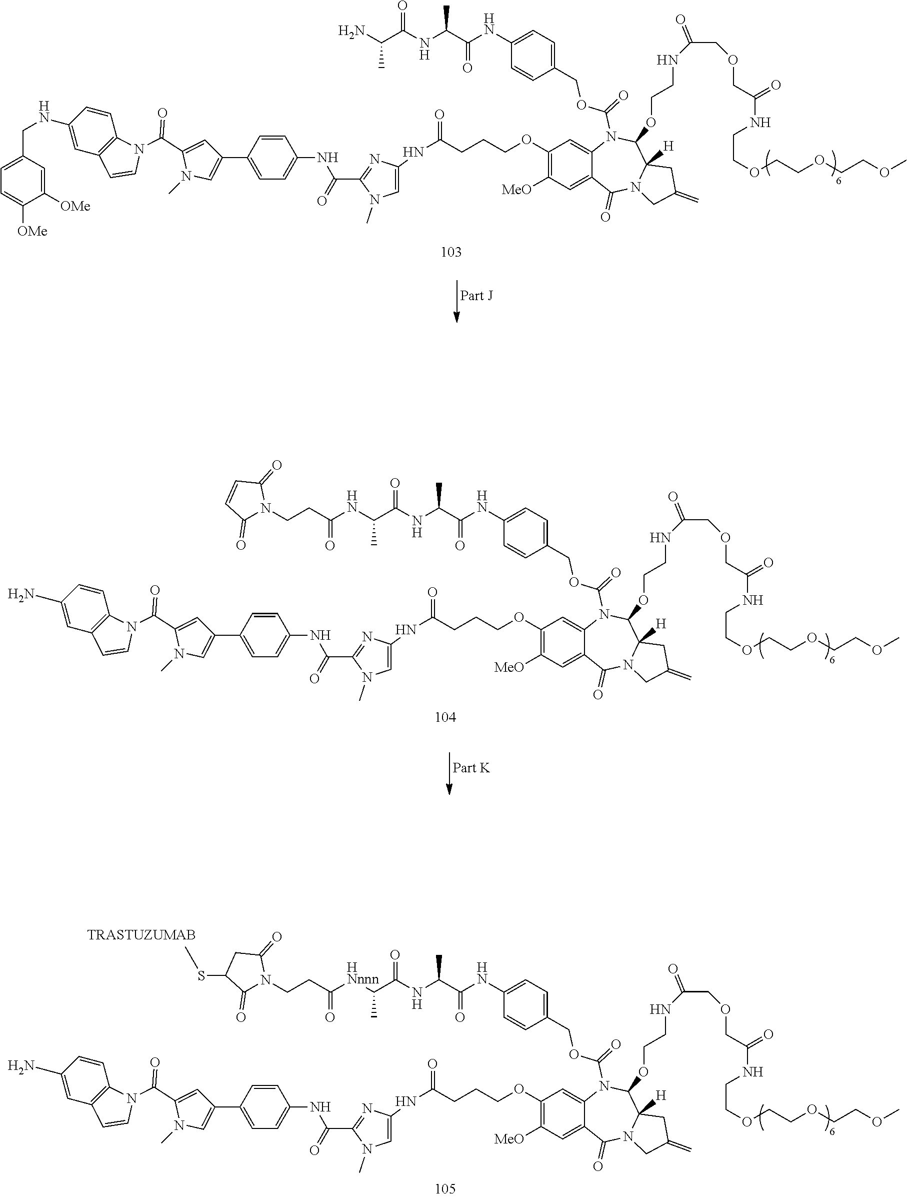

### Part A:

To 3,4-dimethoxybenzaldehyde (2 g, 12.04 mmol) was added DCM (120 mL), 1H-indol-5-amine (1.75 g, 13.24 mmol), NaBH(OAc)₃ (3.57 g, 16.85 mmol) and HOAc (0.78 mL, 13.24 mmol). The reaction mixture was stirred for 72 hours at room temperature, then quenched with saturated aqueous NaHCO₃(100 mL). The aqueous layer extracted with MTBE (3×50 mL). The combined organic layers were dried over Na₂SO₄, then concentrated. The crude product was purified on silica gel (0-10% MeOH in DCM) to afford compound 95 (2.47 g, 72.6% yield) as a pale-yellow solid. ESI-MS calc for C₁₇H₁₉N₂O₂⁺ (M+H) 283.1; found 283.1.

### Part B:

To compound 95 (1.183 g, 4.19 mmol) was added acetone (6.98 mL), H₂O (6.98 mL), NaHCO₃(0.352 g, 4.19 mmol) and allyl (2,5-dioxopyrrolidin-1-yl) carbonate (0.834 g, 4.19 mmol). The reaction mixture was stirred for 12 hours at room temperature, then diluted with H₂O (50 mL) and DCM (50 mL). The aqueous layer was extracted with DCM (3×20 mL). The combined organic layers were dried over Na₂SO₄, then concentrated. The crude product was purified on silica gel (0-10% MeOH in DCM) to provide compound 96 (1.37 g, 89% yield) as a red-brown oil. ESI-MS calc for C₂₁H₂₃N₂O₄ (M+H) 367.2. found 367.1.

### Part C:

To 4-(4-(4-(tert-butoxycarbonylamino)-1-methyl-H-imidazole-2-carboxamido)phenyl)-1-methyl-H-pyrrole-2-carboxylic acid (1 g, 2.275 mmol, was added HCl (4.0 M in dioxane, (17.07 mL, 68.3 mmol), and the reaction mixture was stirred for 4 days at room temperature, then additional HCl (4.0 M in dioxane, 24 mL, 96 mmol) was added. After 3 more days the reaction mixture was concentrated under reduced pressure to provide 4-(4-(4-amino-1-methyl-1H-imidazole-2-carboxamido)phenyl)-1-methyl-1H-pyrrole-2-carboxylic acid (0.772 g, 100% yield). ESI-MS calc for CHisN5O₃ (M+H) 340.1; found 340.1.

To 4-(4-(4-amino-1-methyl-1H-imidazole-2-carboxamido)phenyl)-1-methyl-1H-pyrrole-2-carboxylic acid (0.772 g, 2.275 mmol) was added DCM (22.75 mL) and DIEA (0.396 mL, 2.275 mmol). The mixture was stirred at room temperature for 5 minutes the 2,5-dioxopyrrolidin-1-yl (2-(trimethylsilyl)ethyl) carbonate (0.590 g, 2.275 mmol), was added. After 24 hours, additional of 2,5-dioxopyrrolidin-1-yl (2-(trimethylsilyl)ethyl) carbonate (295 mg, 1.14 mmol) and DIEA (1.14 mmol, 200 μL) were added. After 24 hours, the reaction mixture was concentrated under reduced pressure. The crude product was purified on silica gel (0-45% MeOH in DCM) and then by reverse phase MPLC (10-100% MeCN in H₂O with 0.1% HOAc) to provide compound 97 (0.648 g, 58.9% yield). ESI-MS calc for C₂₃H₃₀N₅O₅Si⁺ (M+H) 484.2; found 484.1.

### Part D:

To compound 97 (0.648 g, 1.340 mmol) was added DMF (14.3 mL) and di(H-imidazol-1-yl)methanone (0.261 g, 1.608 mmol), and the reaction mixture stirred at room temperature for 3 hours at which time LC/MS indicated formation of the intermediate 2-(trimethylsilyl)ethyl (2-((4-(5-(1H-imidazole-1-carbonyl)-1-methyl-1H-pyrrol-3-yl)phenyl)carbamoyl)-1-methyl-1H-imidazol-4-yl)carbamate (0.695 g, 100% yield). ESI-MS calc for C₂₆H₃₂N₇O₄Si⁺ (M+H) 534.2; found 534.2.

To the intermediate was added DBU (0.100 mL, 0.670 mmol) and compound 96 (0.491 g, 1.340 mmol) in DMF (3 mL), and the reaction mixture was stirred at room temperature for 5 hours. Additional DBU (0.100 mL, 0.670 mmol) and compound 96 (0.491 g, 1.340 mmol) in 3 mL DMF (3 mL) was added, and the reaction mixture was stirred for 2 days. Then, a third portion of DBU (0.100 mL, 0.670 mmol) and compound 96 (0.491 g, 1.340 mmol) in DMF (3 mL) was added, and the stirring was continued for 3 more days. The reaction mixture was concentrated and the crude product was purified on silica gel (0-10% MeOH in DCM) to provide compound 98 (880 mg, 79% yield). ESI-MS calc for C₄₄HoN-0sSi (M+H) 832.3; found 832.2.

### Part E:

To compound 98 (0.7103 g, 0.854 mmol) was added THF (8.54 mL) and tetrabutylammonium fluoride (1.0 M in THF, 1.024 mL, 1.024 mmol) and the reaction mixture was stirred at room temperature. After 2 hours, additional tetrabutylammonium fluoride (0.7 mmol, 700 μL) solution was added. After 2 hours the reaction mixture was concentrated, and the crude product was purified on silica gel (0-10% MeOH in DCM) to provide compound 99 (285 mg, 48.5% yield). ESI-MS calc for C₃₈H₃₈N₇O₆⁺ (M+H) 688.3; found 688.2.

### Part F (BDJ4-016 and BDJ4-018)

To compound 82 (0.4 g, 0.588 mmol) was added (9H-fluoren-9-yl)methyl (2-hydroxyethyl)carbamate (5.83 g, 20.57 mmol), THF (11.75 mL), and chlorotrimethylsilane (0.746 mL, 5.88 mmol). The reaction mixture was heated to 50° C. and stirred for 4 hours, and then concentrated. The crude product was filtered through silica gel (5-25% MeOH in DCM) to provide impure Fmoc-protected compound 100 (0.556 g, 100% yield). ESI-MS calc for C₅IHs6NsO3 (M+H) 946.4; found 946.3.

To the Fmoc-protected compound 100 (0.556 g, 0.588 mmol) was added DCM (94 mL) and piperidine (23.51 mL). The reaction mixture was stirred at room temperature for 1 hour and then concentrated. The residue was purified on silica gel (0-25% MeOH in DCM) to provide compound 100 (0.425 g, 0.588 mmol, 100% yield). ESI-MS calc for C₃₆H₄₆NsOi (M+H) 724.3; found 724.3.

### Part G:

To compound 100 (0.084 g, 0.116 mmol) in DMF (1.16 mL) was added 1,4-dioxane-2,6-dione (0.013 g, 0.116 mmol) and the reaction mixture was stirred at room temperature under argon for 12 hours to provide a solution of the carboxylic acid intermediate (0.097 g, 100% yield) in DMF. ESI-MS calc for C₄₀H₅₀N₅O₁₅⁺ (M+H) 840.3; found 840.3.

To the solution of the carboxylic acid intermediate (0.097 g, 0.116 mmol) in DMF (1.16 mL) was added 2,5,8,11,14,17,20,23-octaoxapentacosan-25-amine (0.044 g, 0.116 mmol), HOAt (0.017 g, 0.128 mmol), DIEA (0.051 mL, 0.290 mmol), and HATU (0.053 g, 0.139 mmol). The reaction mixture was stirred at room temperature for 72 hours. Additional HATU (0.1 g, 0.263 mmol) and DIEA (100 μL, 0.575 mmol) were added and the reaction mixture was stirred for 2 hours at room temperature, and then concentrated. The crude product was purified on silica gel (0-25% MeOH in DCM) to provide compound 101 (0.067 g, 47.9% yield). ESI-MS calc for C₇H₈₅N₆O₂₂ (M+H) 1205.6; found 1205.5.

### Part H:

To compound 101 (0.089 g, 0.074 mmol) was added pyrrolidine (0.018 mL, 0.222 mmol), triphenylphosphine (4.84 mg, 0.018 mmol), DCM (1.477 mL) and tetrakis(triphenylphosphine)palladium(O) (8.53 mg, 7.38 μmol). The reaction mixture was stirred for 1 hour at room temperature, and the crude product was purified on silica gel (0-50% MeOH in DCM) to provide the amine intermediate (0.0356 g, 43.0% yield). ESI-MS calc for C₅₃H₈₁N₆O₂₀⁺ (M+H) 1121.6; found 1121.4.

To the amine intermediate (0.0356 g, 0.032 mmol) was added HOAt (4.32 mg, 0.032 mmol), Alloc-Ala-OH (6.05 mg, 0.035 mmol, prepared as described above) DMF (1.588 mL), DIEA (0.019 mL, 0.111 mmol) and HATU (0.014 g, 0.038 mmol). The reaction mixture was stirred for 12 hours at room temperature, and then concentrated. The crude product was purified on silica gel (0-25% MeOH in DCM) to provide the methyl ester of compound 102 (0.0326 g, 80% yield). ESI-MS calc for C₆₀H₉₀N₇O₂₃⁺ (M+H) 1276.6; found 1276.5.

To the methyl ester of compound 102 (0.0326 g, 0.026 mmol) was added KOH (7.16 mg, 0.128 mmol) in MeOH (2.128 mL) and H₂O (0.426 mL), the resulting mixture was stirred for 18 hours at room temperature before being acidified to pH˜4-5 by the dropwise addition of glacial HOAc, and then concentrated. The crude product was purified by reverse phase MPLC (10-100% MeCN in H₂O with 0.1% HOAc) to provide compound 102 (0.022.7 g, 70.4% yield). ESI-MS calc for C₅₉H₈₈N₇O₂₃⁺ (M+H) 1262.6; found 1262.5.

### Part I:

To compound 102 (0.0227 g, 0.018 mmol) was added HOAt (2.448 mg, 0.018 mmol), compound 99 (0.012 g, 0.018 mmol), DMF (3.60 mL), DIEA (9.40 μl, 0.054 mmol) and HATU (8.20 mg, 0.022 mmol). The reaction mixture was stirred for 3 hours at room temperature, and then additional compound 99 (0.005 g, 0.007 mmol) and DIEA (10 μL, 0.057 mmol) were added, and the reaction mixture stirred for 12 hours at room temperature. Then, compound 99 (0.005 g, 0.007 mmol), HOAt (0.001 g, 7.4 μmol), HATU (0.003 g, 7.9 μmol) and DIEA (10 μL, 0.057 mmol) were added and the reaction mixture was stirred an additional 3 hours at room temperature before being concentrated. The crude product was purified on silica gel (0-30% MeOH in DCM) to provide bis-alloc-protected compound 103 (0.0337 g, 97% yield). ESI-MS calc for C₉₇H₁₂₃N₁₄O₂₈⁺ (M+H) 1931.9; found 1931.7.

To bis-alloc-protected compound 103 (0.0337 g, 0.017 mmol) was added triphenylphosphine (1.144 mg, 4.36 μmol), pyrrolidine (5.01 μl, 0.061 mmol), DCM (1.744 mL) and tetrakis(triphenylphosphine)palladium(O) (2.016 mg, 1.744 μmol). The reaction mixture was stirred at room temperature for 45 minutes then additional tetrakis(triphenylphosphine)palladium(O) (2.016 mg, 1.744 μmol) was added, and the stirring continued for 3 hours at room temperature when, a third portion of tetrakis(triphenylphosphine)palladium(O) (2.016 mg, 1.744 μmol) was added and the reaction was stirred for 1 more hour at room temperature before concentration. The crude product was purified by reverse phase MPLC (10-100% MeCN in H₂O with 0.1% HOAc) to provide compound 103 (0.016 g, 52.0% yield). ESI-MS calc for C₈₉H₅N₁₄₀₂₄* (M+H) 1763.8; found 1763.8.

### Part J:

To compound 103 (0.016 g, 9.07 μmol) was added 2,5-dioxopyrrolidin-1-yl 3-(2,5-dioxo-2,5-dihydro-1H-pyrrol-1-yl)propanoate (2.415 mg, 9.07 μmol) and DIEA (4.74 μl, 0.027 mmol) in DMF (1.296 mL), and the reaction mixture was stirred at room temperature for 1 hour. The crude product was purified by reverse phase MPLC (10-100% MeCN in H₂O with 0.1% HOAc) to provide N-DMB-protected compound 104 (0.0048 g, 2.506 μmol, 27.6% yield). ESI-MS calc for C₉₆H₁₂₀N₁₅O₂₇⁺ (M+H) 1914.8: found 1914.8.

To N-DMB-protected compound 104 (0.0048 g, 2.506 μmol) was added DCM (4.75 mL) and H₂O (0.264 mL). Then DDQ (1 mg/mL) in DCM (5×100 μL) at a rate of 1 portion per hour (a total of 0.5 mg, 2.20 μmol, 0.87 eq of DDQ) was added. The reaction mixture was concentrated and the crude product was purified by reverse phase MPLC (10-100% MeCN in H₂O with 0.1% HOAc) to provide compound 104 (0.002 g, 45.2% yield). ESI-MS calc for C₈₇H₁₁₀N₁₅O₂₅⁺ (M+H) 1764.8; found 1764.7.

### Part K:

Conjugate 105 was prepared from Trastuzumab and compound 104 as described in Example 3. The purified conjugate 105 had a PBD to trastuzumab ratio of 4.1 as determined by UV-Vis using molar extinction ε_{330 nm}==37,456.3 cm⁻¹ M⁻¹ and C_{280 nm}=27,081 cm⁻¹ M⁻¹ for the corresponding compound without C11 modification (prepared in a similar fashion to compound 105) and ε_{280 nm}=226,107 cm⁻¹ M⁻¹ for trastuzumab).

### Example 22. Synthesis of Trastuzumab Conjugate 112

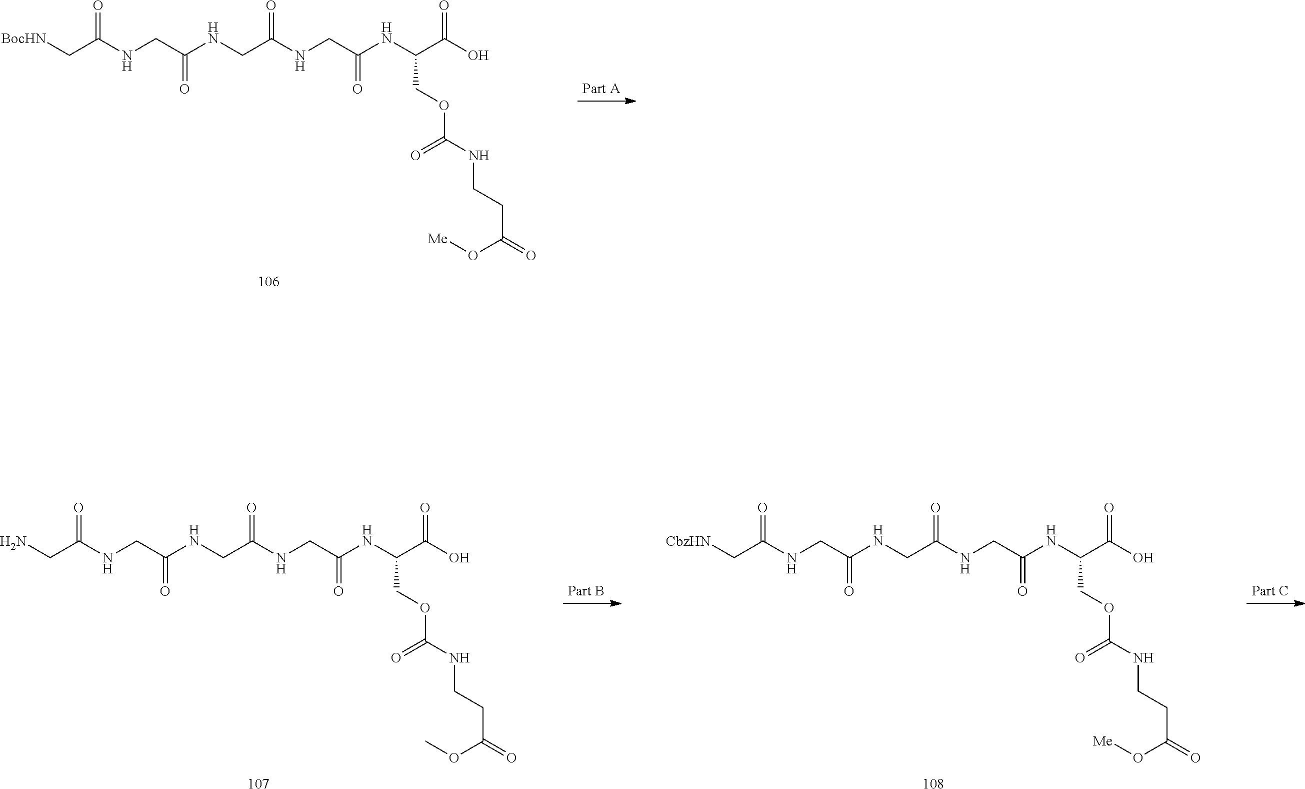 [SMILES: COC(=O)CCNC(=O)OC[C@H](NC(=O)CNC(=O)CNC(=O)CNC(=O)CNC(=O)OC(C)(C)C)C(=O)O ; "106" ; COC(=O)CCNC(=O)OC[C@H](NC(=O)CNC(=O)CNC(=O)CNC(=O)CN)C(=O)O ; COC(=O)CCNC(=O)OC[C@H](NC(=O)CNC(=O)CNC(=O)CNC(=O)CNC(=O)OCc1ccccc1)C(=O)O ; "107" ; "108"]

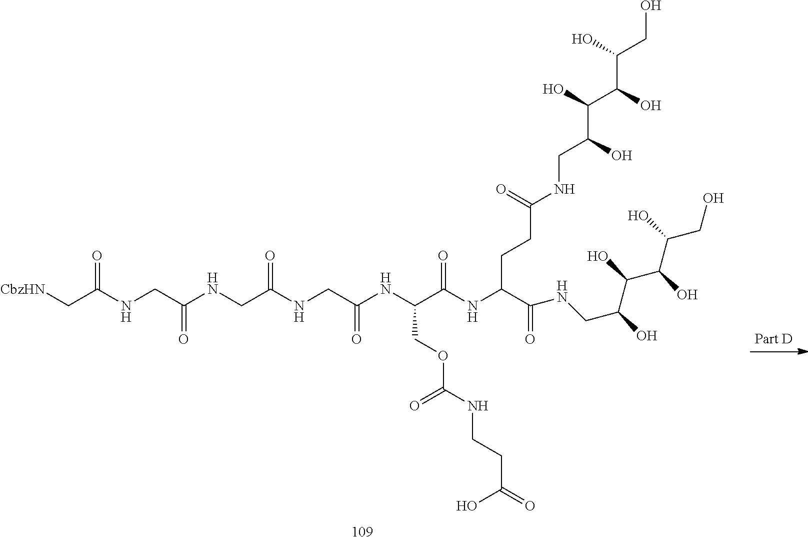 [SMILES: O=C(O)CCNC(=O)OC[C@H](NC(=O)CNC(=O)CNC(=O)CNC(=O)CNC(=O)OCc1ccccc1)C(=O)NC(CCC(=O)NC[C@H](O)[C@@H](O)[C@H](O)[C@H](O)CO)C(=O)NC[C@H](O)[C@@H](O)[C@H](O)[C@H](O)CO ; "109"]

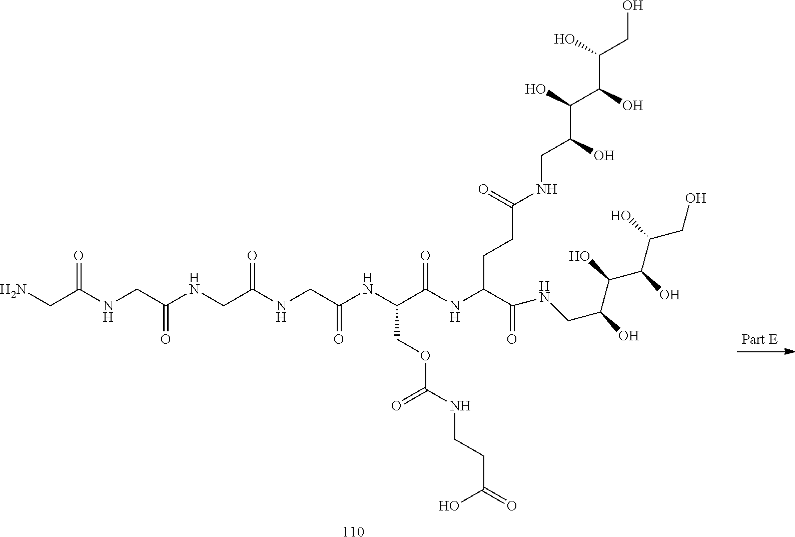 [SMILES: NCC(=O)NCC(=O)NCC(=O)NCC(=O)N[C@@H](COC(=O)NCCC(=O)O)C(=O)NC(CCC(=O)NC[C@H](O)[C@@H](O)[C@H](O)[C@H](O)CO)C(=O)NC[C@H](O)[C@@H](O)[C@H](O)[C@H](O)CO ; "110"]

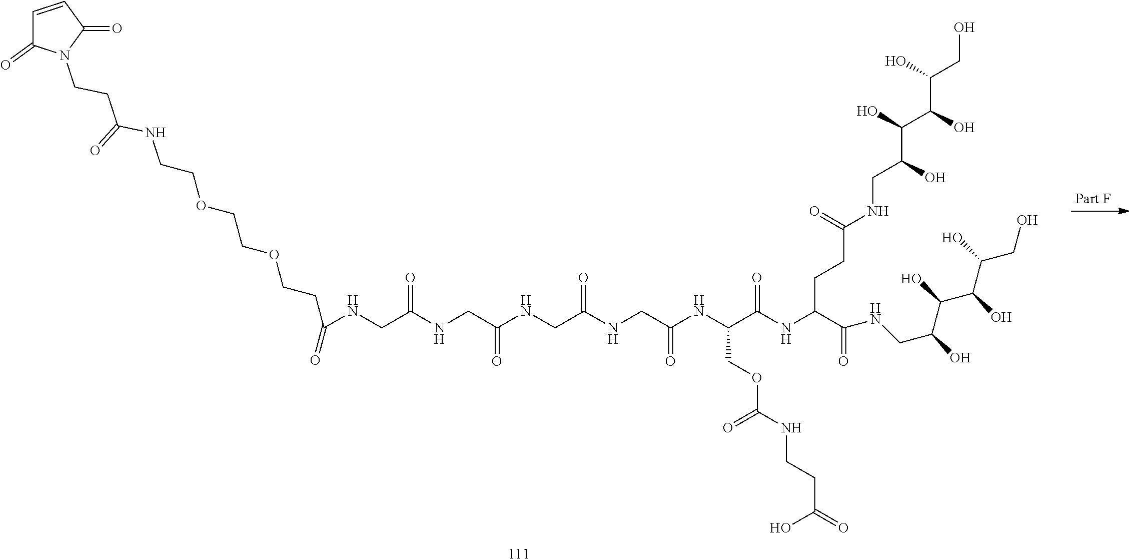 [SMILES: O=C(O)CCNC(=O)OC[C@H](NC(=O)CNC(=O)CNC(=O)CNC(=O)CNC(=O)CCOCCOCCNC(=O)CCN1C(=O)C=CC1=O)C(=O)NC(CCC(=O)NC[C@H](O)[C@@H](O)[C@H](O)[C@H](O)CO)C(=O)NC[C@H](O)[C@@H](O)[C@H](O)[C@H](O)CO ; "111"]

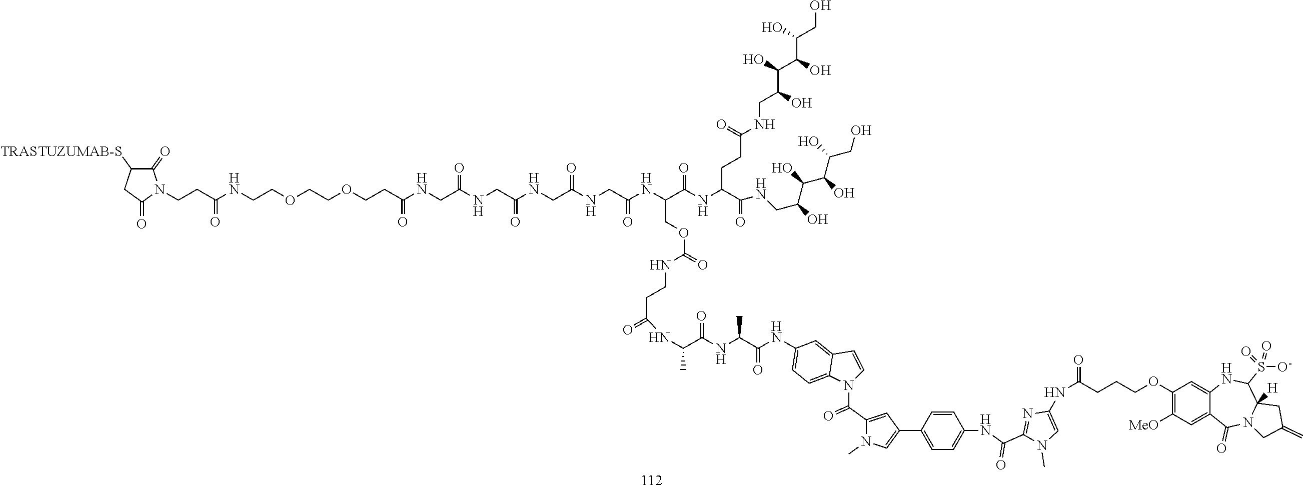

### Part A:

A solution of compound 106 (4.4 g, 7.82 mmol, prepared as described in U.S. patent Ser. No. 15/819,650) in DCM (20 ml) was added TFA (5 ml, 64.9 mmol), the reaction mixture stirred at room temperature for 1 hour, then concentrated to afford compound 107 (5.7 g, 126% yield) and used directly in the next step. ESI MS calc for C₁₆H₂₇N₆O₁₀ (M+H) 463.18; found 463.2.

### Part B:

To compound 107 (3.63 g, 7.86 mmol) in DMF was slowly added TEA (1.64 mL, 11.79 mmol) followed by Cbz-OSu (Z-succinimide) (2.155 g, 8.65 mmol) in DMF (5 mL). After 4 hours at room temperature the solution was concentrated to ˜10 mL volume then ethyl ether (35 mL) was added to give compound 108 (3.6 g, 6.03 mmol, 77% yield) as a solid. ESI MS calc for C₂₄H₃₄N₆₀₁₂ (M+H) 597.2; found 597.2.

### Part C:

To compound 108 (500 mg, 0.838 mmol) and compound 87 (476 mg, 1.0 mmol) in DMF (18 ml) at 0° C., HOBt (25.7 mg, 0.168 mmol) and 3-(((ethylimino)methylene)amino)-N,N-dimethylpropan-1-amine hydrochloride (177 mg, 0.922 mmol) was added in one portion and stirred at 0° C. for 5 min, that at room temperature for 1 h. The crude product was purified by RP-HPLC (0-80% acetonile in water) to afford the methyl ester of compound 109 (350 mg, 39.7% yield) as a white solid. ESI MS calc for C₄₁H₆₇N₉₀₂₃ (M+H) 1052.4; found 1052.3.

To a solution of the methyl ester of compound 109 (833 mg, 0.792 mmol) in DMF was added a solution of 35% HCl (2 mL) in water (9 mL) and the reaction mixture was stirred overnight. Additional 35% HCl (4 mL) was added and the reaction mixture was stirred at room temperature for 3 hours, concentrated, adjusted to pH 4-5 using saturated NaHCO₃ and the crude product was purified by RP-HPLC (0-80% acetonile in water) afford compound 109 as a colorless solid (125 mg, 15% yield).

### Part D:

To a solution of compound 109 (210 mg, 0.202 mmol) in a mixture of water and ethanol (1:1, 10 mL) was added 2 drop of 10% HCl. To the resulting mixture was added Pd—C (10%, 15 mg). The reaction mixture was stirred overnight at room temperature under hydrogen. The mixture was filtered and concentrated to afford compound 110 (200 mg, 109% yield) as a yellow solid. ESI-MS calc for C₃₂H₅₈N₉O₂₁ 904.37; found 904.34.

### Part E:

To a solution of compound 110 (100 mg, 0.111 mmol) in DMF (2 ml) were added 2,5-dioxopyrrolidin-1-yl 3-(2-(2-(3-(2,5-dioxo-2,5-dihydro-1H-pyrrol-1-yl)propanamido)ethoxy)ethoxy)propanoate (47.1 mg, 0.111 mmol) followed by TEA (0.046 ml, 0.332 mmol). After 2 hours additional 2,5-dioxopyrrolidin-1-yl 3-(2-(2-(3-(2,5-dioxo-2,5-dihydro-1H-pyrrol-1-yl)propanamido)ethoxy)ethoxy)propanoate 47.1 mg, 0.111 mmol) and TEA (0.046 ml, 0.332 mmol) was added and the mixture was stirred 40 min. The crude product was purified by RP-HPLC (0.1% HOAc buffer acetonitrile/water) to afford compound 111 (70 mg, 52% yield). ESI-MS calc for C₄₆H₇₆N₁₁O₂₇ 1214.49; found 1214.45.

### Part H:

Conjugate 112 was prepared as described in Example 10 except that compound 111 was used instead of compound 8. The purified conjugate 112 had a PBD to trastuzumab ratio of 4.2 as determined by UV-Vis using molar extinction E_{330 nm}=38858.5 cm⁻¹M⁻¹ 1 and ε_{280 nm}=29820.413 cm⁻¹M⁻¹ for compound 9 and ε_{280 nm}=226,107 cm⁻¹M⁻¹ for trastuzumab).

### Example 23: Synthesis of Trastuzumab Conjugate 115

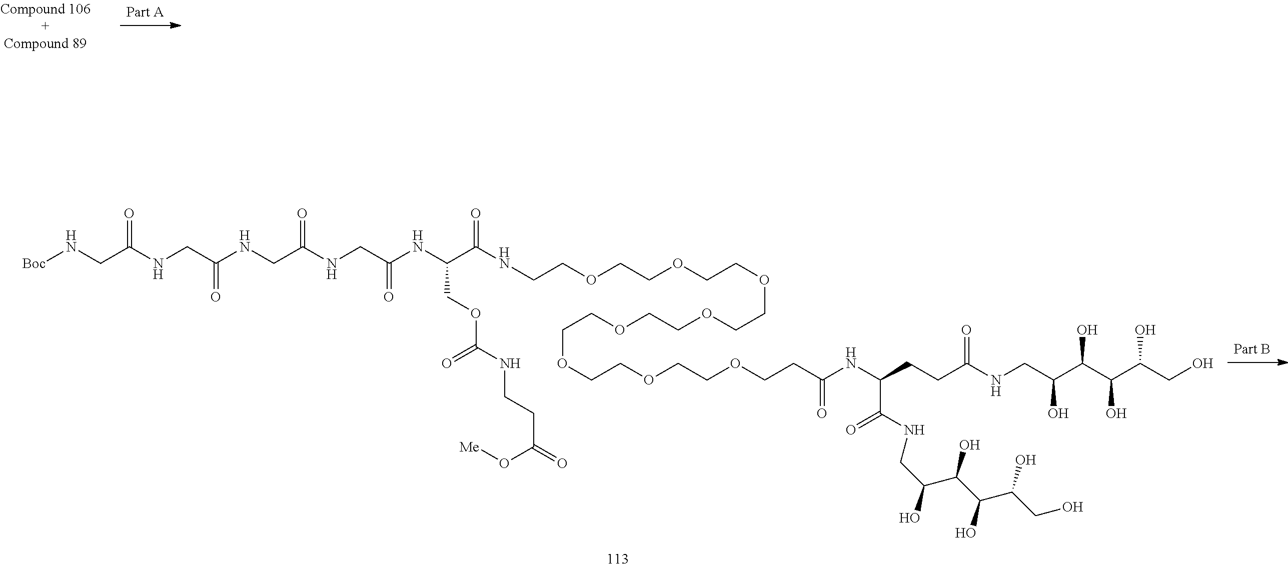

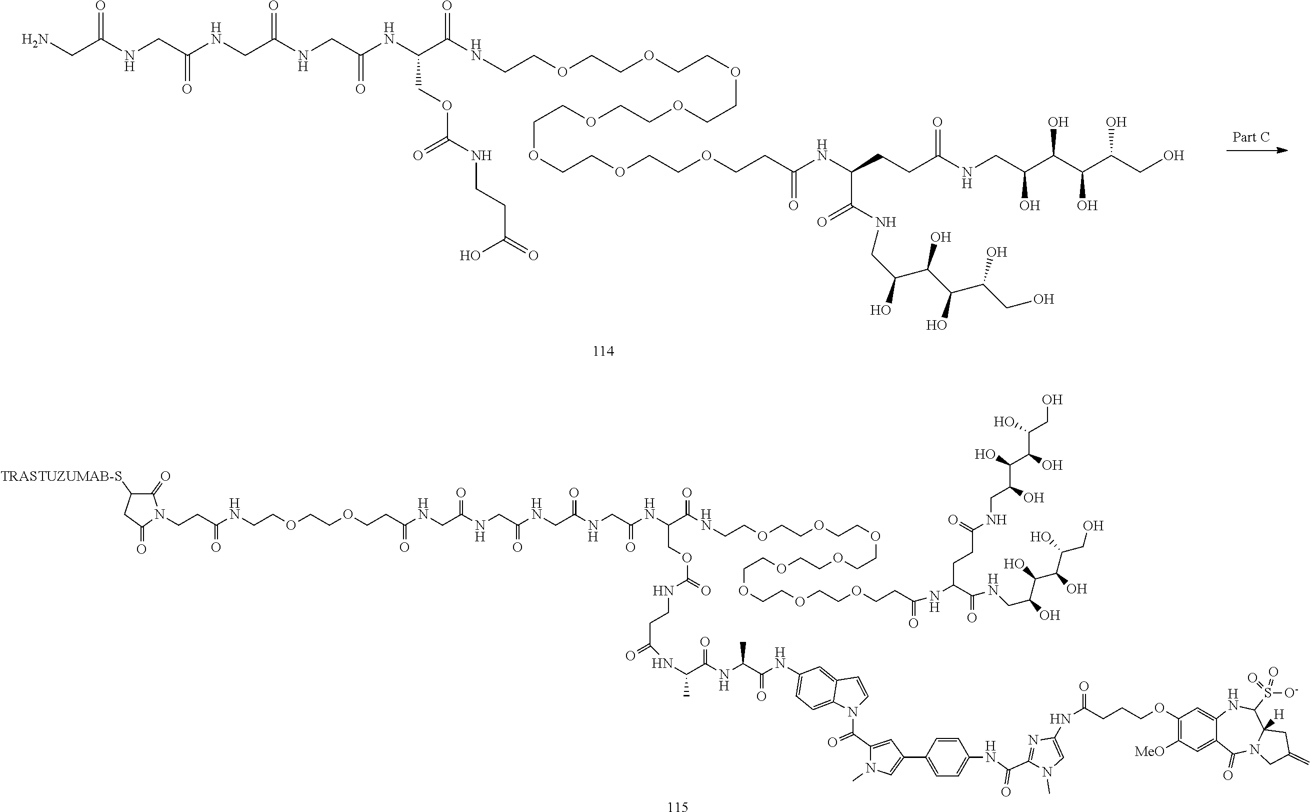

### Part A:

Compound 113 was prepared according to the procedure described in Example 22 for the synthesis of compound 109 except that compound 89 was used instead of compound 87 to afford compound 113 (240 mg, 3.9% yield) as a colorless solid. ESI-MS calc for C₅₇H₁₀₅N₁₀O₃₂ 1441.69: found 1441.61.

### Part B:

Compound 113 (240 mg, 0.166 mmol) was dissolved in 8% HCl (2 ml) and stirred at overnight at room temperature. The crude product was purified by RP-HPLC to afford compound 114 (76 mg, 35% yield). ESI-MS calc for C₅₁H₉₅N₁₀O₃₀ 1327.62; found 1327.56.

### Part C:

Conjugate 115 was prepared as described in Example 22, Part E and Part F, except that compound 114 was used instead of compound 110. The purified conjugate 115 had a PBD to trastuzumab ratio of 3.9 as determined by UV-Vis using molar extinction ε300 nm=38858.5 cm⁻¹ M⁻¹ and c_{280 nm}=29820.413 cm⁻¹ M⁻¹ for compound 9 and C_{280 nm}=226,107 cm⁻¹M⁻¹ for trastuzumab).

### Example 24: Synthesis of Trastuzumab Conjugate 119

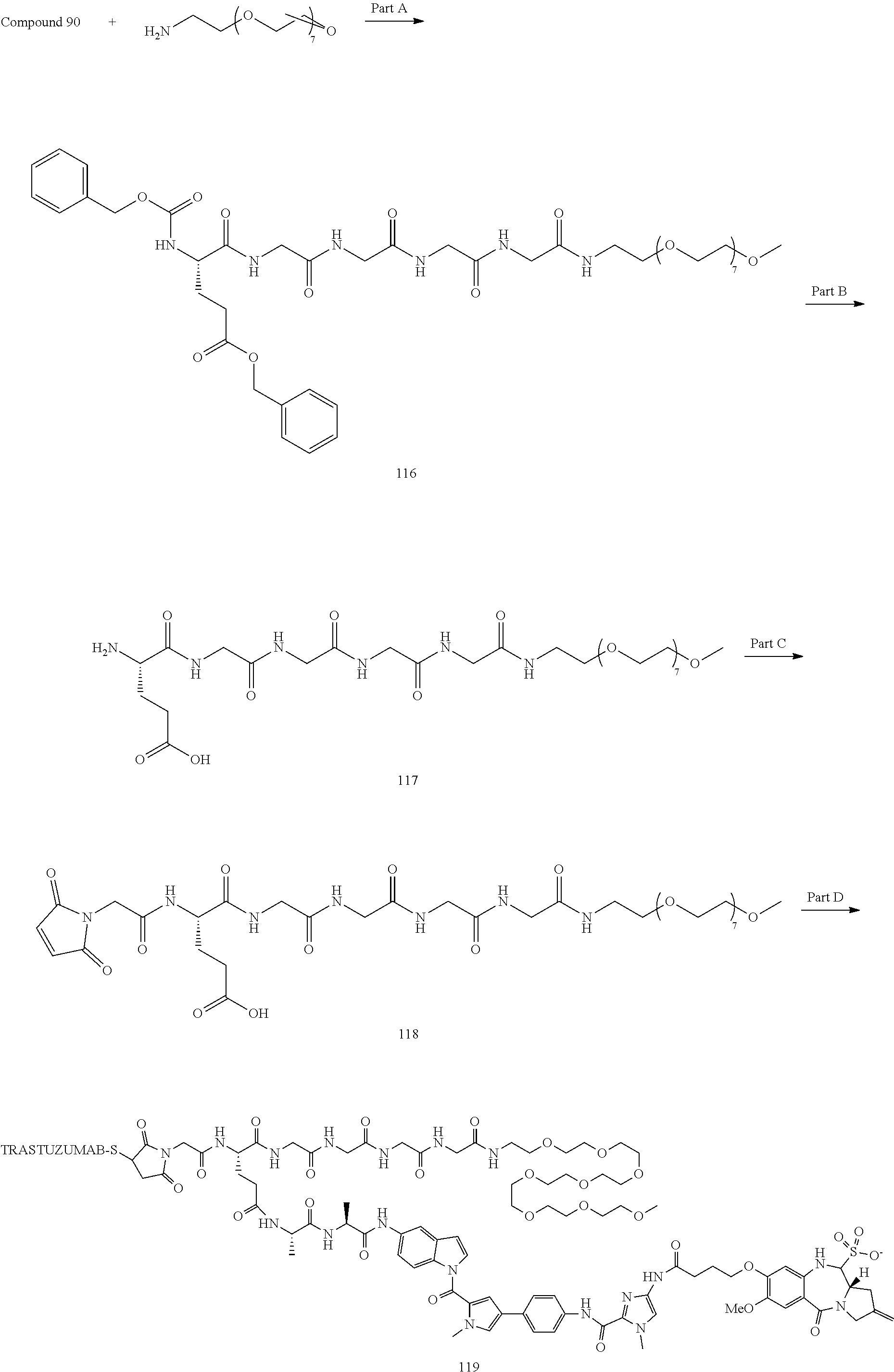

### Part A:

To a suspension of compound 90 (328 mg, 0.547 mmol) in DMF (7.5 mL) was added 2,5,8,11,14,17,20,23-octaoxapentacosan-25-amine (252 mg, 0.656 mmol) followed by HATU (250 mg, 0.656 mmol) and DIEA (0.287 ml, 1.641 mmol), the reaction mixture was stirred overnight. The crude product was purified by RP-HPLC (acetonitrile/water buffered with 0.1% TFA) to obtain compound 116 as a white amorphous solid (480 mg, 91% yield). ESI MS calc for C₄₅H₆₉N₆₀₁₇ (M+H) 965.47; found 965.43.

### Part B:

To compound 116 (480 mg, 0.497 mmol) in ethanol (50 ml) and water (5.00 ml) under argon was added Pd/C (132 mg, 0.124 mmol) and the mixture was hydrogenation at 30 psi H₂. After 16 hours the reaction mixture was filtered through celite, concentrated. to afford compound 117 as a colorless oil (336 mg, 91% yield). ESI MS calc for C₃₀H₅₇N₆O₁₅ (M+H) 741.39; found 741.37.

### Part C:

Compound 117 (150 mg, 0.202 mmol), 2,5-dioxopyrrolidin-1-yl 2-(2,5-dioxo-2,5-dihydro-1H-pyrrol-1-yl)acetate (51.1 mg, 0.202 mmol) and triethylamine (0.028 ml, 0.202 mmol) in DCM (10 ml) were stirred at 0° C. After 1 hour DMF (1 ml) was added and the pH adjusted to pH 8-9 with triethylamine. After 4 hours acetic acid (0.464 ml, 8.10 mmol) was added, the reaction mixture was concentrated and purified by RP-HPLC (acetonitrile/water buffered with 0.1% AcOH) to afford compound 118 as a white amorphous solid (56 mg, 32% yield). ESI MS calc for C₃₆H₆₀N₇O₁₈ (M+H) 878.40; found 878.37.

### Part D:

Conjugate 119 was prepared as described in Example 10 except that compound 118 was used instead of compound 8. The purified conjugate 119 had a PBD to trastuzumab ratio of 3.2 as determined by UV-Vis using molar extinction ε_{330 nm}=38858.5 cm⁻¹ M⁻¹ and ε_{280 nm}=29820.413 cm⁻¹ M⁻¹ for compound 9 and ε_{280 nm}=226,107 cm⁻¹ M⁻¹ for trastuzumab).

### Example 25: Synthesis of Trastuzumab Conjugate 122

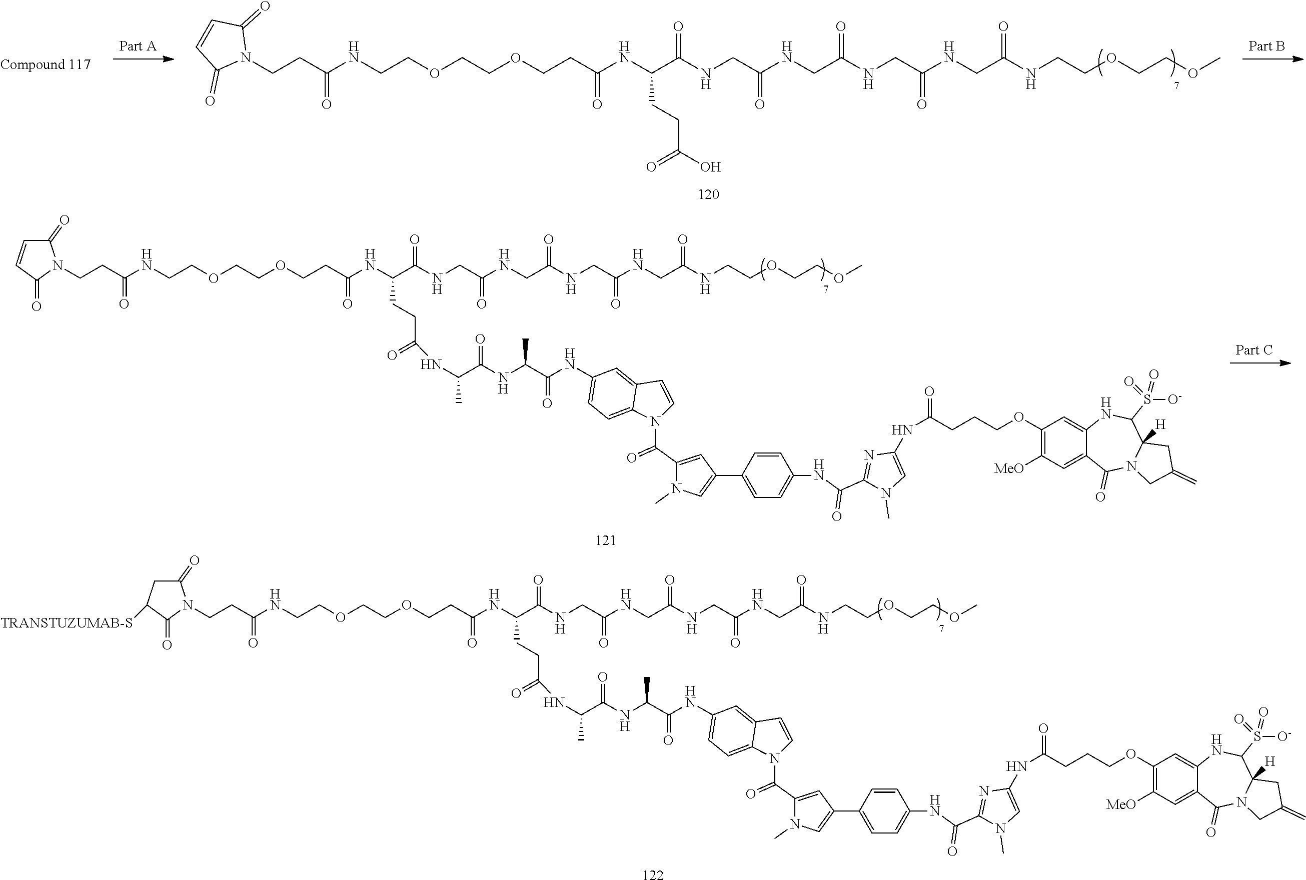

### Part A:

A solution of compound 117 (163 mg, 220 μmol), 2,5-dioxopyrrolidin-1-vl 3-(2-(2-(3-(2,5-dioxo-2,5-dihydro-1H-pyrrol-1-yl)propanamido)ethoxy)ethoxy)propanoate (103 mg, 242 μmol), TEA (34 μL, 242 μmol) and DMF (2 mL) was stirred at room temperature for 4.5 hours. The reaction mixture was concentrated to give the crude compound 120 (250 mg) as an off-white foam that was used in the next step without further purification. ESI MS calc for C₄₄H₇₄N₈O₂₁ (M+H) 1051.5. found 1051.4.

### Part B:

A solution of compound 120 (250 mg, 30 μmol), compound 58 (38 mg, 36 μmol), NMP(1 mL), NHS (5 mg, 45 μmol), EDCl.HCl (9 mg, 45 μmol), DIEA (8 μL, 45 μmol) was stirred at room temperature for 19 hours. The reaction mixture was concentrated and purified by preperative HPLC (10-100% acetonitrile/water containing 0.1% formic acid), to afford compound 121 (11 mg, 19% yield) as a white, fluffy solid. ESI MS calc for C₉₃H₁₂₃N₁₉O₂₈ (M+H) 1956.1; found 1955.8.

### Part C:

Conjugate 122 was prepared as described in Example 10 except that compound 121 was used instead of compound 59. The purified conjugate 122 had a PBD to trastuzumab ratio of 3.5 as determined by UV-Vis using molar extinction ε_{330 nm}=38858.5 cm⁻¹ M⁻¹ and ε_{280 nm}=29820.413 cm⁻¹M⁻¹ 1 for compound 9 and ε_{280 nm}=226,107 cm⁻¹ M⁻¹ for trastuzumab).

### Example 26: Synthesis of Trastuzumab Conjugate 130

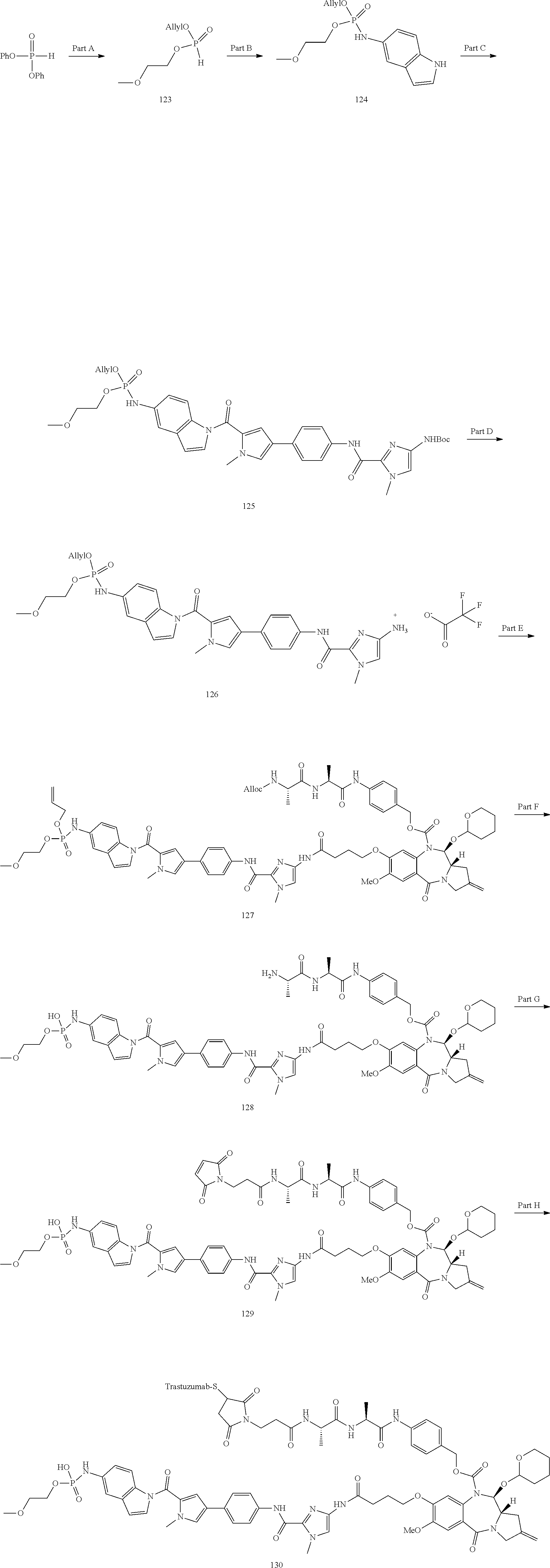

PartA

To diphenylphosphite (40.2 mL, 210 mmol) was added pyridine (13.6 mL) and 2-methoxyethan-1-ol (13.25 mL, 168 mmol). The reaction mixture was stirred at room temperature for 2 hours then pyridine (13.6 mL) and prop-2-en-1-ol (11.43 mL, 168 mmol), was added and stirring continued for 12 hours at room temperature. The crude product was purified on silica gel (0-100% EtOAc in hexanes) to provide compound 123 (15.927 g, 52.6% yield) as a clear liquid. ESI-MS calc for C₆H₁₄O₄P′ (M+H) 181.1; found 181.1.

### Part B:

To 1H-indol-5-amine (1.13 g, 8.55 mmol) was added DIEA (1.489 mL, 8.55 mmol) and 16 mL of CC14 (16 mL). The mixture was cooled to 0° C. and then a solution of compound 123 (1.540 g, 8.55 mmol) in CC14 (5 mL) was added. The reaction mixture was stirred at 0° C. for 30 min, then allowed to warm to room temperature and stir for 1 hour. The crude product was purified on silica gel (0-30% MeOH in DCM) to afford compound 124 (1.573 g, 59.3% yield). ESI-MS calc for C₁₄H₂₀N₂O₄P⁺ (M+H) 311.1; found 311.1.

### Part C:

To tert-butyl 2-(4-(5-(1H-imidazole-1-carbonyl)-1-methyl-1H-pyrrol-3-yl)phenylcarbamoyl)-1-methyl-1H-imidazol-4-ylcarbamate (0.4 g, 0.910 mmol,) was added DMF (3.03 mL) and di(1H-imidazol-1-yl)methanone (0.221 g, 1.365 mmol) and the reaction mixture was stirred for 12 hours at room temperature to form the imidazole adduct intermediate (0.446 g, 0.910 mmol, 100% yield). ESI-MS calc for C₂₅H₂₈N₇O₄⁺ 490.2; found 490.2.

To the solution of the imidazole adduct (0.446 g, 0.910 mmol) in DMF (3.03 mL) was added a solution of compound 124 (0.282 g, 0.910 mmol) and DBU (0.068 mL, 0.455 mmol) in DMF (1.6 mL) and the reaction mixture was stirred at room temperature for 12 hours. The concentreated crude product was purified on silica gel (0-10% MeOH in DCM) to provide compound 125 (0.25 g, 37.5% yield). ESI-MS calc for C₃₆H₄₃N₇O₈P⁺ (M+H) 732.3; found 732.2.

### Part D:

To a solution of compound 125 (200 mg, 0.273 mmol) in dichloromethane (2.278 ml was added TFA (456 μl and the mixture stirred at room temperature for 1 hour, concentrated, diluted with ethyl acetate and concentrated again. The resulting residue ws dissolved residue in ACN and lyophillized to afford compound 126 (˜200 mg) as a brown solid. ESI-MS calc for C₃H₃₅N₇₀₆P+(M+H) 632.24; found 632.19.

### Part E:

To a solution of compound 84 (630 mg, 0.754 mmol) in MeOH (6 mL) was added a solution of potassium carbonate (104 mg, 0.754 mmol) in water (200 μL). The mixture was stirred at room temperature for 2 days, then neutralized with 10% HCl, extracted with DCM, the organic extracts were dried over Na₂SO₄, concentrated and purified on silica gel (0-20% methanol in DCM) to afford the desired carboxylic acid intermediate (210 mg, 33.9% yield) as a colorless solid.

To a mixture of the carboxylic acid intermediate (200 mg, 0.244 mmol) was added compound 126 (200 mg, 0.317 mmol), HATU (102 mg, 0.268 mmol), HOAt (36.5 mg, 0.268 mmol) and TEA (0.068 ml, 0.487 mmol). The reaction mixture was stirred overnight, concentrated and purified on silica gel to afford compound 127 (35 mg, 10% yield). ESI-MS calc for C₇₂H₈₄N₁₂O₁₈P (M+H) 1435.57; found 1435.48.

### Part F:

To a solution of compound 127 (35 mg, 0.024 mmol) in DCM (2 mL) and pyrrolidine (6.01 μl, 0.073 mmol) was added tetrakistriphenylphosphine palladium (2.82 mg, 2.438 μmol) under argon. The reaction mixture was stirred at room temperature for about hour. The crude product was purified by RP-HPLC to afford compound 128 (20 mg, 63%). ESI-MS calc for C₆H, 6N₁₂₀₁₆P (M+H) 1311.52; found 1311.44.

### Part G:

To a solution of compound 128 (20 mg, 0.015 mmol) in DMF (1 mL) was added 2,5-dioxopyrrolidin-1-yl 3-(2,5-dioxo-2,5-dihydro-1H-pyrrol-1-yl)propanoate (6.09 mg, 0.023 mmol) and TEA (5.31 μl, 0.038 mmol). After 40 minutes, the pH was adjusted to pH 3-4 with HOAc. The crude product was purified by RP-HPLC to afford compound 129 (13.5 mg, 61% yield). ESI-MS calc for C₇₂H₈₁N₃O₁₉P (M+H) 1462.51; found 1462.49.

### Part H:

Conjugate 130 was prepared as described in Example 3 except that compound 129 was used instead of compound 1. The purified conjugate 130 had a PBD to trastuzumab ratio of 4.5 as determined by UV-Vis using molar extinction ε_{330 nm}=26,410 cm⁻¹ M⁻¹ and ε_{280 nm}=18,910 cm⁻¹ M⁻¹ for 129 and ε_{280 nm}=226,107 cm⁻¹ M⁻¹ for trastuzumab).

### Example 27: Synthesis of XMT-1535 Conjugate 135

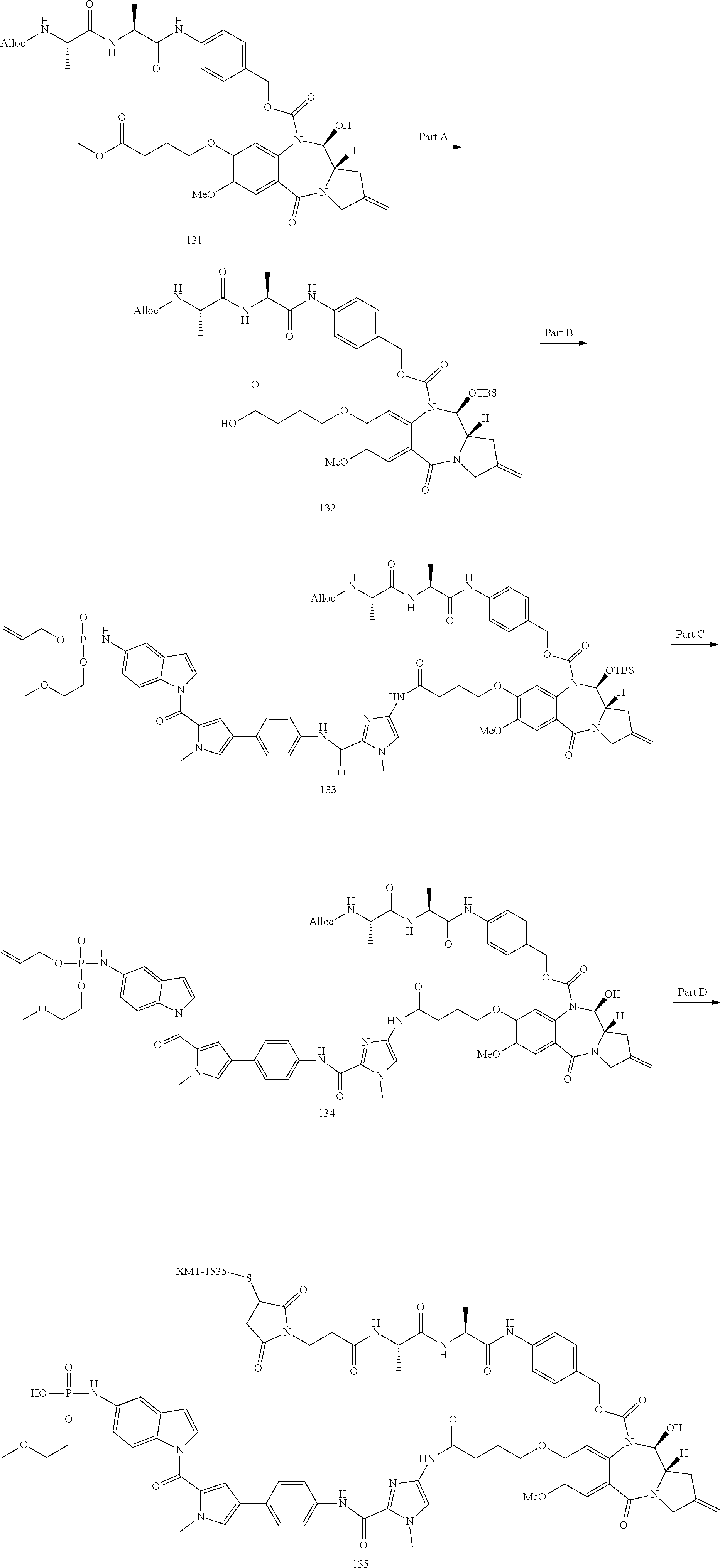

### Part A:

To a solution of compound 131 (280 mg, 0.372 mmol) in dichloromethane (10 mL) at 0C under Ar was slowly added t-butyldimethylsilyltrifluoromethanesulfonate (0.257 mL, 1.117 mmol) followed by 2,6-lutidine (0.130 mL, 1.117 mmol). The reaction mixture was stirred at room temperature for 1 hour. The crude product was purified on silica gel (0-10% MeOH in DCM) to provide an intermediate product (180 mg, 55.8% yield) as a yellow solid. ESI-MS calc for C₄₃HoNsO₂Si (M+H) 866.4; found 866.4.

To a solution of the intermediate product (1.59 g, 1.84 mmol) in MeOH (68 mL) was added NaOH (0.2N, 36.6 mL, 7.36 mmol). The reaction mixture stirred at room temperature for 1 hour. LCMS indicated that the reaction was complete. pH of the reaction mixture was adjusted to 3 with 1N HCl and the organic phase was washed with DCM (3×20 mL). The combined organic phases were dried over Na₂SO₄ before concentrated in vacuum. The residue was purified on silica gel (ISCO, 40 g column, 0-10% MeOH in DCM) to provide compound 132 as a yellow foam (861 mg, 1.01 mmol, 55% yield). ESI-MS calc for C₄₂HSNO2Si (M+H) 852.4; found 852.4.

### Part B:

A mixture of compound 132 (350 mg, 0.411 mmol), HOAt (84 mg, 0.616 mmol) and HATU (234 mg, 0.616 mmol) in DCM (20 mL) was stirred for 10 min at 0° C. The reaction mixture was then added to compound 126 (288 mg, 0.411 mmol) followed by the addition of DIEA (0.143 mL, 0.822 mmol). The reaction mixture was stirred overnight at room temperature and then washed with brine. The organic phase was dried over Na₂SO₄ concentrated in vacuum, and the residue was purified on silica gel (ISCO, 80 g column, 0-10% MeOH in DCM) to provide compound 133 as a yellow foam (400 mg, 0.273 mmol, 66%). ESI-MS calc for C₇₃H₉₀N₁₂O₁₇PSi (M+H) 1465.6; found 1465.7.

### Part C:

A solution of compound 133 (100 mg, 0.068 mmol) in THF (2 mL) was added a mixture solution of tetra-n-butylammonium fluoride (0.955 mL, 0.955 mmol) and acetic acid (0.062 mL, 1.092 mmol). The reaction mixture was stirred at room temperature overnight. The resulting solution was concentrated in vacuum and purified on silica gel (0-10% MeOH in DCM) to provide compound 134 (75 mg, 0.055 mmol, 81% yield). ESI-MS calc for C₆₇H₇₆N₁₂₀₁₇P (M+H) 1351.5; found 1351.6.

### Part D:

Conjugate 135 was prepared as described in Example 26 except that compound 134 was used instead of compound 127 and XMT-1535 was used instead of trastuzumab. The purified conjugate 135 had a PBD to XMT-1535 ratio of 4.0 as determined by UV-Vis using molar extinction ε_{330 nm}=26,410 cm⁻¹M⁻¹ and ε280 nm=18,910 cm⁻¹M⁻¹ for 127 and ε280 nm=207,405.77 cm⁻¹ M⁻¹ for XMT-1535).

### Example 27A: Synthesis of Rituximab Conjugate 135A

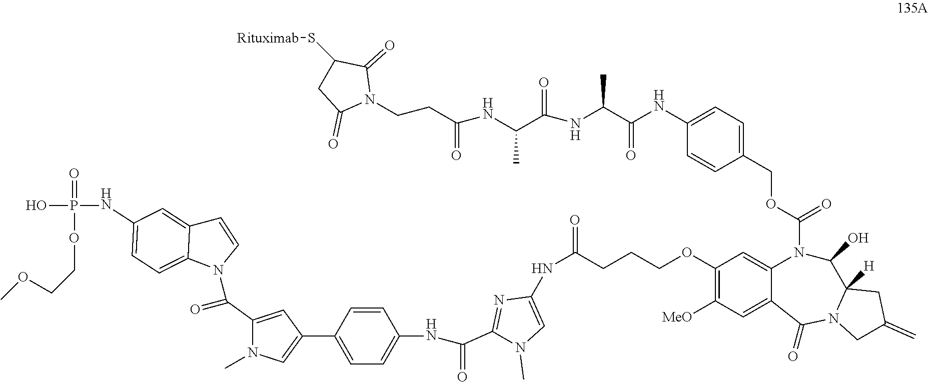

Conjugate 135A was prepared as described above in Example 27 except that Rituximab was used instead of XMT-1535. The purified conjugate 135A had a PBD to Rituximab ratio of 5.2 as determined by UV-Vis using molar extinction ε_{330 nm}=26,410 cm-1M⁻¹ and E_{280 nm}=18,910 cm⁻¹M⁻¹ for 127 and ε_{280 nm}=228,263 cm⁻¹M⁻¹ for Rituximab).

### Example 28: Synthesis of XMT-1535 Conjugate 136

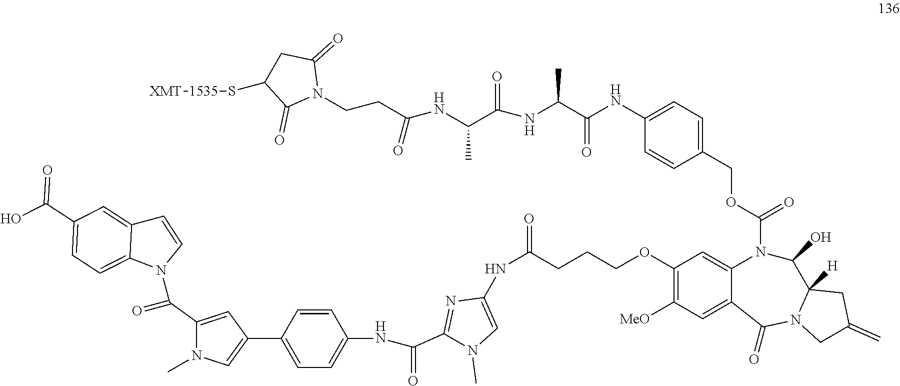

Conjugate 136 was prepared as described in Example 9 except that compound 132 was used instead of compound 52 and XMT-1535 was used as the PBRM. The purified conjugate 136 had a PBD to XMT-1535 ratio of 3.5 as determined by UV-Vis using molar extinction ε_{330 nm}=31,180.8 cm⁻¹M⁻¹ and ε_{280 nm}=24,632.8 cm⁻¹ M⁻¹ for compound 56 and 6280 nm=207,405.77 cm⁻¹ M⁻¹ for XMT-1535).

### Example 28A: Synthesis of Rimuximah Conjugate 136A

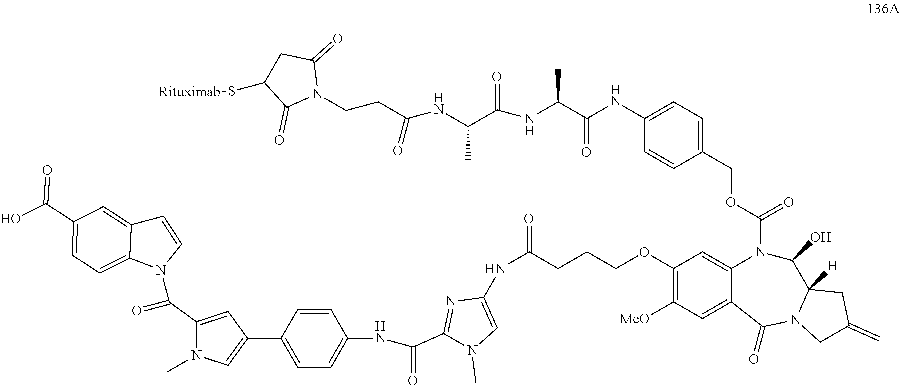

Conjugate 136A was prepared as described above in Example 28 except that Rituximab was used instead of XMT-1535. The purified conjugate 136A had a PBD to Rituximab ratio of 3.6 as determined by UV-Vis using molar extinction E_{330 nm}=26,410 cm-1M⁻¹ and E_{280 nm}=18,910 cm⁻¹M⁻¹ for 127 and ε_{280 nm}=228,263 cm⁻¹ M⁻¹ for Rituximab).

### Example 29: Cell Viability Assay for Conjugates

PBD conjugates were evaluated for their antiproliferation properties in tumor cell lines in vitro using CellTiter-Glo® (Promega Corp). Cells were plated in black walled 96-well plate and allowed to adhere overnight at 37° C. in a humidified atmosphere of 5% CO₂. BT474, SKBR3, NCI-N₈₇ cells (HER2 expressing cells), JIMT1 cells (HER2 medium expression level cells), MCF7 cells (HER2 low expressing levels cells), Calu3 cells (non-small-cell lung cancer cell line), DLD1 (colorectal adenocarcinoma cell line), HT29 (colon adenocarcinoma cell line) and were plated at a density of 5,000 cells per well and OVCAR3 (ovarian adenocarcinoma cell line, not amplified, ATCC, Cat. #HTB-161) was cultured in RPMI medium with 20% FBS. The next day the medium was replaced with 50 μL fresh medium and 50 μL of 2× stocks of antibody-PBD conjugate were added to appropriate wells, mixed and incubated for 72 h. CellTiter-Glo® reagent was added to the wells at room temperature and the luminescent signal was measured after 10 min using a SpectraMax M5 plate reader (Molecular Devices). Dose response curves were generated using SoftMax Pro software. IC₅₀ values were determined from four-parameter curve fitting.

Table I and Table II give illustrative results for the antiproliferation properties of the PBD conjugates respectively.

TABLE I

| | BT474 | SKBR3 | N87 | JIMT1 | MCF7 | |
|---|---|---|---|---|---|---|
| | IC₅₀ | IC₅₀ | IC₅₀ | IC₅₀ | IC₅₀ | Calu3 IC₅₀ |
| Conjugate No | (nmol/L) | (nmol/L) | (nmol/L) | (nmol/L) | (nmol/L) | (nmol/L) |
| 5 | 300.00 | 0.04 | 1.09 | 34.00 | 300.00 | ND |
| 10 | 0.31 | 0.03 | 0.09 | 0.25 | 10.10 | ND |
| 20 | 0.11 | 0.02 | 0.06 | 0.26 | 300.00 | ND |
| 26 | 0.13 | 0.03 | 0.09 | 0.20 | 2.15 | 0.07 |
| 31 | 0.33 | 0.12 | 0.29 | 1.35 | 2.73 | 0.26 |
| 36 | 0.49 | 0.09 | 0.29 | 1.50 | 2.55 | 0.40 |
| 38 | 0.03 | 0.02 | 0.03 | 0.11 | 1.45 | 0.04 |
| 41 | 2.07 | 0.28 | 1.06 | 4.20 | 5.30 | 0.80 |
| 56 | 0.11 | 0.02 | 0.10 | 300.00 | 300.00 | 0.03 |
| 60 | ND | ND | ND | 0.11 | 0.02 | 0.01 |
| 63A | ND | ND | ND | >100 | ND | ND |
| 67 | 0.23 | 0.02 | 0.15 | 300.00 | 300.00 | 0.02 |
| 71 | 0.17 | 0.02 | 0.12 | 10.74 | 300.00 | 0.03 |
| 73 | 0.16 | 0.02 | 0.13 | 30.60 | 24.18 | 0.02 |
| 79 | 0.30 | 0.03 | 0.31 | 300.00 | 300.00 | 0.04 |
| 86 | ND | ND | ND | 29.01 | 64.66 | 0.02 |
| 94 | ND | ND | ND | 0.07 | 1.35 | 0.02 |
| 94A | ND | ND | ND | 20.5 | ND | ND |
| 94A | ND | ND | ND | 3.19 | ND | ND |
| 105 | ND | ND | ND | 0.04 | 0.91 | 0.02 |
| 112 | ND | ND | ND | 0.13 | 0.68 | 0.02 |
| 115 | ND | ND | ND | 0.18 | 0.89 | 0.03 |
| 119 | ND | ND | ND | 0.49 | 6.36 | 0.06 |
| 122 | ND | ND | ND | 0.14 | 1.37 | 0.02 |
| 130 | ND | ND | ND | 0.07 | 300.00 | 0.02 |
| 135 | ND | ND | ND | >100 | ND | ND |
| 136 | ND | ND | ND | >100 | ND | ND |

TABLE II

| | DLD1 | HT29 | OVCAR3 |
|---|---|---|---|
| Conjugate | IC₅₀ | IC₅₀ | IC₅₀ |
| No | (nmol/L) | (nmol/L) | (nmol/L) |
| 61 | 0.21 | 0.02 | ND |
| 62 | 1.72 | 1.37 | ND |
| 63 | 0.71 | 0.02 | ND |
| 63A | ND | ND | 0.04 |
| 64 | 16.81 | 4.02 | ND |
| 73A | ND | ND | 0.05 |
| 94A | ND | ND | 0.08 |
| 94A | ND | ND | 0.03 |
| 135 | ND | ND | 0.02 |
| 136 | ND | ND | 0.16 |

ND=not determined

As shown in Tables I and II, the antibody-drug conjugates show efficacy in the tested cell lines.

### Example 30: Tumor Growth Response to Administration of Antibody-Polymer-Drug Conjugates

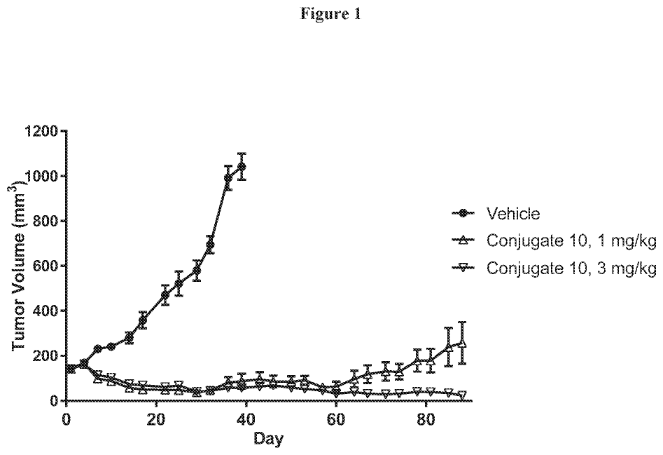

Female CB-17 SCID mice were inoculated subcutaneously with Calu3 cells, DLFD1 cells, NCI-N87 cells, OVCAR-3 tumor fragments or HT-29 tumor fragments (n=10 for each group). Test compound or vehicle were dosed IV as a single dose on day 1. Tumor size was measured at the times indicated in FIGS. 1 to 5 using digital calipers. Tumor volume was calculated and was used to determine the delay in tumor growth. Mice were sacrificed when tumors reached a size of 800 mm³. Tumor volumes are reported as the mean SEM for each group.

FIG. 1 provides the results for the tumor response in mice inoculated subcutaneously with Calu3 cells (n=10 for each group) after IV administration as a single dose on day 1 of vehicle and Conjugate 10 at 1 mg/kg or at 3 mg/kg. The results show that on day 90 Conjugate 10 resulted in 10 partial responses at 3 mg/kg and 9 partial responses at 1 mg/kg.

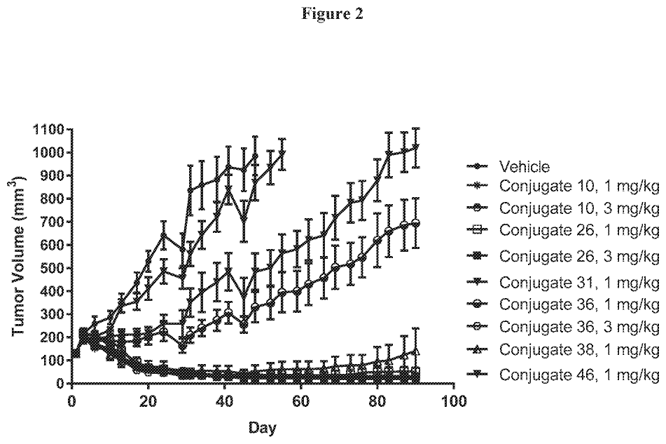

FIG. 2 provides the results for the tumor response in mice inoculated subcutaneously with Calu2 cells (n=10 for each group) after IV administration as a single dose on day 1 of vehicle, Conjugate 10, Conjugate 26 and Conjugate 36 each at 1 mg/kg and at 3 mg/kg, and Conjugate 31, Conjugate 38 and Conjugate 46 at each 1 mg/kg. The results show that on day 90 at 1 mg/kg Conjugate 10 resulted in 7 partial responses, 2 complete responses and 2 tumor free survivors, Conjugate 26 resulted in 8 partial responses and 1 complete response. Conjugate 36 in 9 partial responses and Conjugate 38 in 9 partial responses; and at 3 mg/kg Conjugate 10 resulted in 9 partial responses and 1 complete responses, Conjugate 26 resulted in 9 partial responses, 1 complete response and 1 tumor free survivor and Conjugate 36 in 10 partial responses.

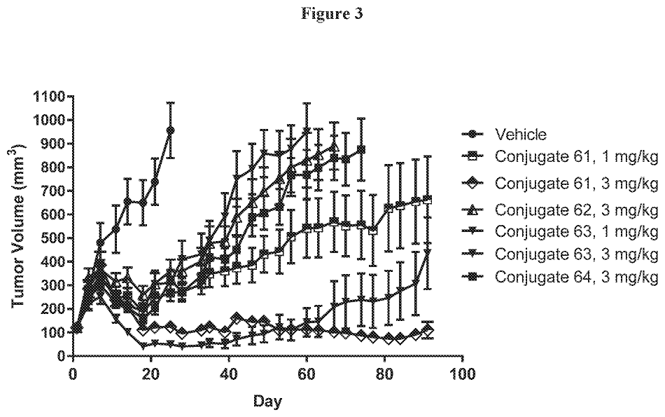

FIG. 3 provides the results for the tumor response in mice inoculated subcutaneously with DLD1 (n=10 for each group) after IV administration as a single dose on day 1 of vehicle, Conjugate 61 and Conjugate 63 each at 1 mg/kg or at 3 mg/kg, and Conjugate 62 and Conjugate 64 each at 3 mg/kg. The results show that on day 90 at 1 mg/kg Conjugate 61 resulted in 2 partial responses, 1 complete response and 1 tumor free survivor; and at 3 mg/kg Conjugate 61 resulted in 5 partial responses, Conjugate 63 resulted in 4 partial responses, 4 complete response and 3 tumor free survivors and Conjugate 64 in 1 complete response and 1 tumor free survivor.

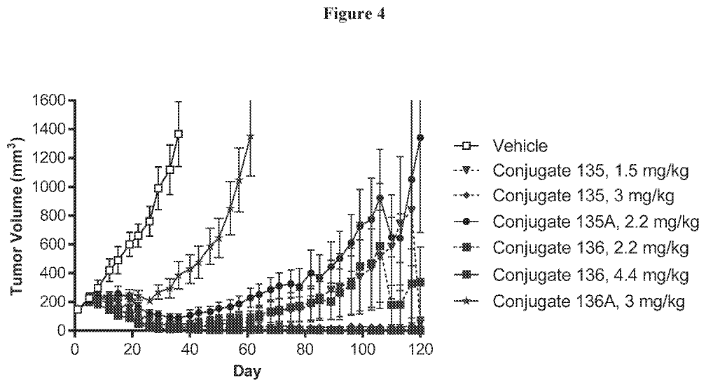

FIG. 4 provides the results for the tumor response in mice subcutaneously implanted with OVCAR-3 tumor fragments (n=10 for each group) after IV administration as a single dose on day 1 of vehicle, Conjugate 135 at 1 mg/kg and at 3 mg/kg, Conjugate 135A at 2.2 mg/kg, Conjugate 136 at 2.2 mg/kg and 4.4 mg/kg, and Conjugate 136A at 3 mg/kg.

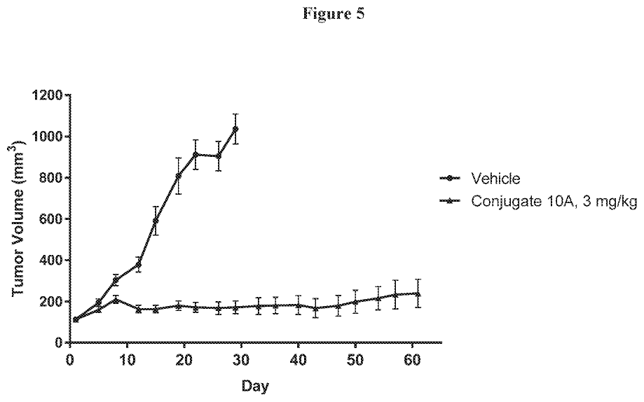

FIG. 5 provides the results for the tumor response in mice subcutaneously implanted with HT-29 tumor fragments (n=10 for each group) after IV administration as a single dose on day 1 of vehicle, Conjugate 10A at 3 mg/kg.

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. An antibody-drug conjugate (ADC) of Formula (I):
PBRM-[L^{C}-D]_{d15} (I)
or a pharmaceutically acceptable salt or solvate thereof, wherein:
PBRM denotes a protein based recognition-molecule;
L^{C} is a linker unit connecting the PBRM to D;
D is a PBD drug moiety; and
d₁₅ is an integer from about 1 to about 20.

2. The conjugate of claim 1, being of Formula (II): 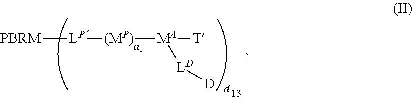
or a pharmaceutically acceptable salt or solvate thereof, wherein:
PBRM denotes a protein based recognition-molecule;
each occurrence of D is independently a PBD drug moiety;
L^{P′} is a divalent linker moiety connecting the PBRM to M^{P}; of which the corresponding monovalent moiety L^{P} contains a functional group W^{P} that is capable of forming a covalent bond with a functional group of the PBRM;
M^{P} is a Stretcher unit;
a₁ is an integer from 0 to 1;
M^{A} comprises a peptide moiety that contains at least two amino acids;
T′ is a hydrophilic group and the 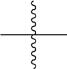
between T′ and M^{A} denotes direct or indirect attachment of T′ and M^{A};
each occurrence of L^{D} is independently a divalent linker moiety connecting D to M^{A} and comprises at least one cleavable bond such that when the bond is broken, D is released in an active form for its intended therapeutic effect; and
d₁₃ is an integer from 1 to 14.

3. The conjugate of any one of the preceding claims, wherein d₁₃ is an integer from 2 to 14, from 2 to 12, from 2 to 10, from 2 to 8, from 2 to 6, from 2 to 4, from 4 to 10, from 4 to 8, from 4 to 6, from 6 to 14, from 6 to 12, from 6 to 10, from 6 to 8, from 8 to 14, from 8 to 12, or from 8 to 10.

4. The conjugate of any one of the preceding claims, wherein d₁₃ is 3 to 5.

5. The conjugate of any one of the preceding claims, wherein d₁₃ is 4 or 5.

6. The conjugate of any one of the preceding claims, wherein L^{P}, when not connected to PBRM, comprises a terminal group W^{P}, in which each W^{P} independently is: 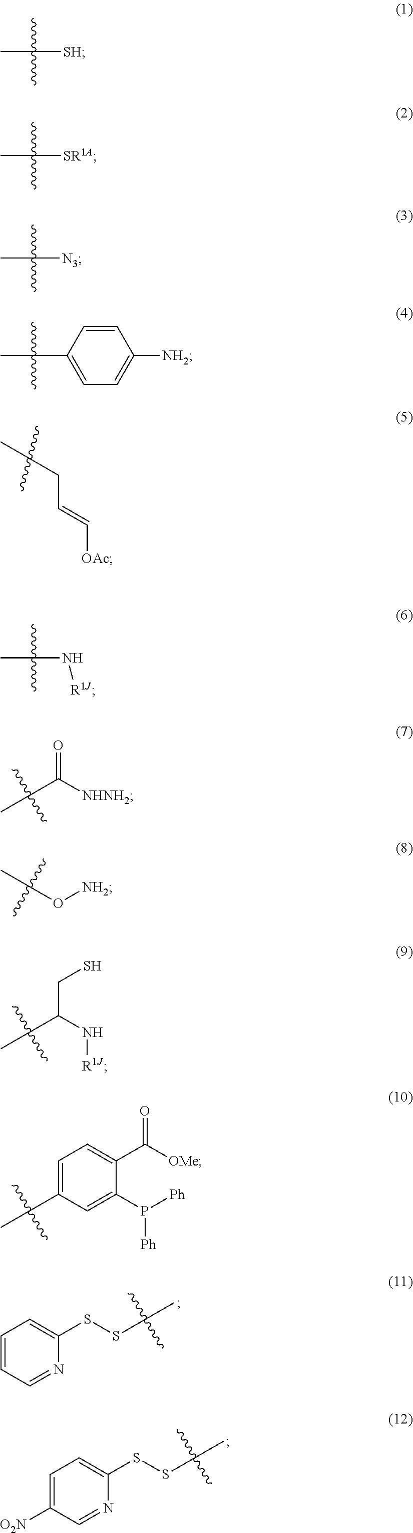 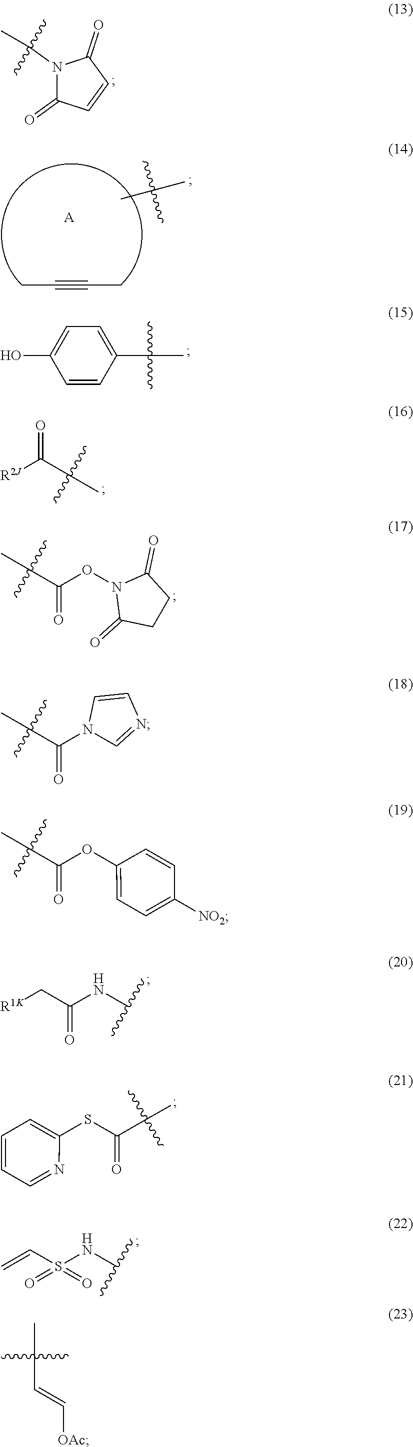 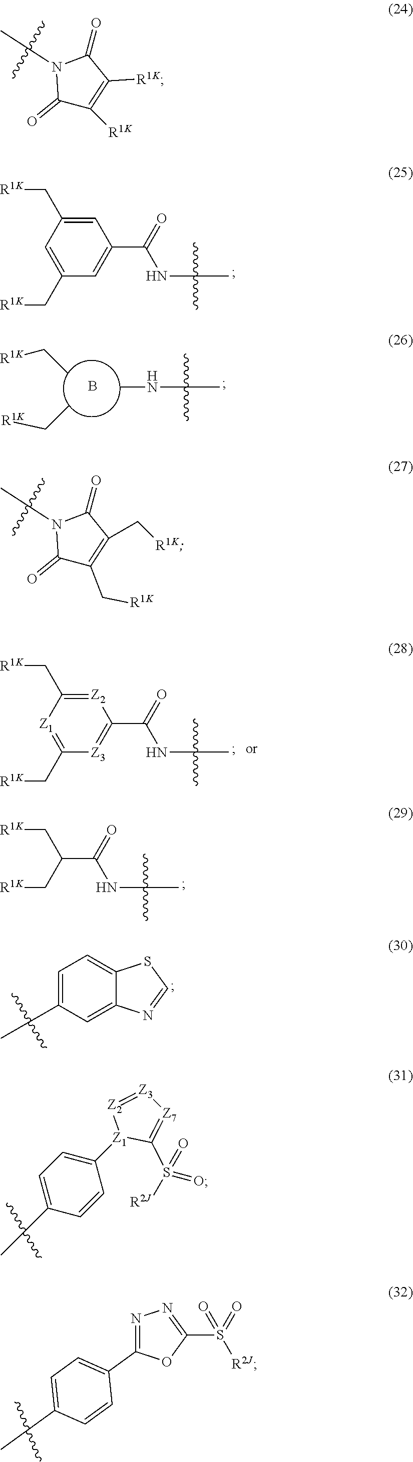 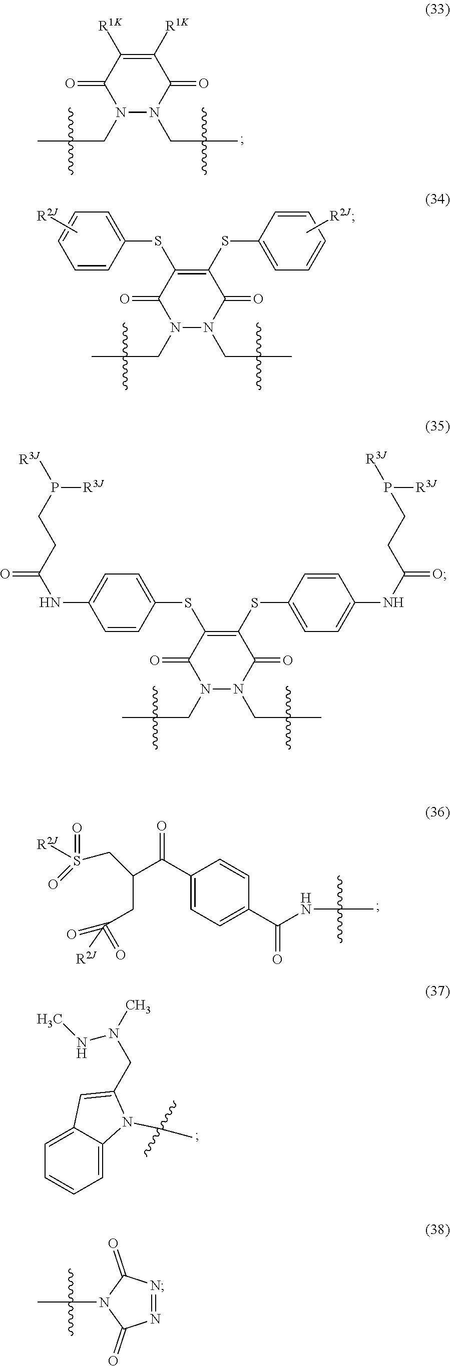 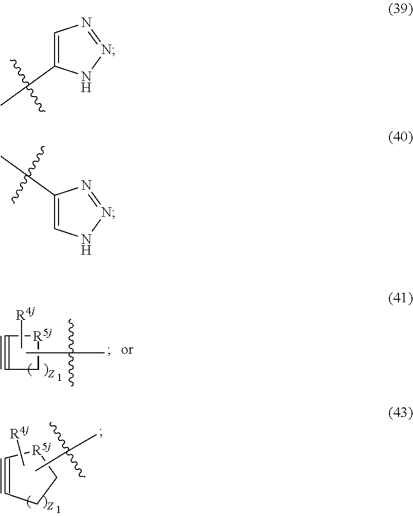
wherein
R^{1K} is a leaving group;
R^{1A} is a sulfur protecting group;
ring A is cycloalkyl or heterocycloalkyl;
ring B is cycloalkyl, heterocycloalkyl, aryl, or heteroaryl;
R^{1J} is hydrogen or an aliphatic, heteroaliphatic, carbocyclic, or heterocycloalkyl moiety;
R^{2J} is hydrogen or an aliphatic, aryl, heteroaliphatic, or carbocyclic moiety;
R^{3J} is C₁₋₆ alkyl;
Z₁, Z₂, Z₃ and Z₇ are each independently a carbon or nitrogen atom;
R^{4j} is hydrogen, halogen, OR, —NO₂, —CN, —S(O)₂R, C₁₋₂₄ alkyl (e.g., C₁₋₆ alkyl), or 6-24 membered aryl or heteroaryl, wherein the C₁₋₂₄ alkyl (e.g., C₁₋₆ alkyl), or 6-24 membered aryl or heteroaryl, is optionally substituted with one or more aryl or heteroaryl; or two R^{4j} together form an annelated cycloalkyl, heterocycloalkyl, aryl, or heteroaryl; R is hydrogen, alkyl, heteroalkyl, cycloalkyl, or heterocycloalkyl
R is hydrogen or an aliphatic, heteroaliphatic, carbocyclic, or heterocycloalkyl moiety;
R^{5j} is C(R^{4j})₂, O, S or NR; and
z₁ is an integer 1, 2, 3, 4, 5, 6, 7, 8, 9 or 10.

7. The conjugate of any one of the preceding claims, wherein each R^{1K} is halo or RC(O)O— in which R is hydrogen or an aliphatic, heteroaliphatic, carbocyclic, or heterocycloalkyl moiety.

8. The conjugate of any one of the preceding claims, wherein each R^{1A} independently is 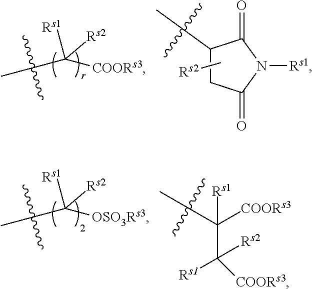
in which r is 1 or 2 and each of R^{s1}, R^{s2}, and R^{s3} is hydrogen or an aliphatic, heteroaliphatic, carbocyclic, or heterocycloalkyl moiety.

9. The conjugate of any one of the preceding claims, wherein L^{P}, when not connected to PBRM is

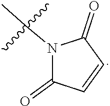 [SMILES: *N1C(=O)C=CC1=O]

10. The conjugate of any one of the preceding claims, wherein M^{P}, when present, is —(Z₄)—[(Z₅)—(Z₆)]_{z}—, with Z₄ connected to L^{P′} or L^{P} and Z₆ connected to L^{M}; in which
z is 1, 2, or 3;
Z₄ is: 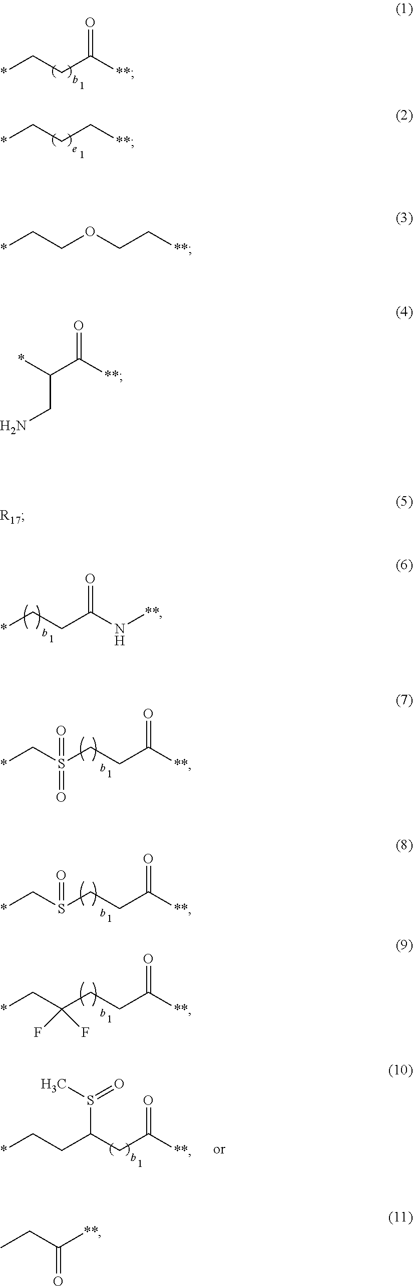
wherein * denotes attachment to L^{P′} or L^{P} and ** denotes attachment to Z₅ or Z₆ when present or to M^{A} when Z₅ and Z₆ are both absent;
b₁ is an integer from 0 to 6;
e₁ is an integer from 0 to 8,
R₁₇ is C₁₋₁₀ alkylene, C₁₋₁₀ heteroalkylene, C₃₋₈ cycloalkylene, O—(C₁₋₈ alkylene, arylene, —C₁₋₁₀ alkylene-arylene-, -arylene-C₁₋₁₀ alkylene-, —C₁₋₁₀ alkylene-(C₃₋₈ cycloalkylene)-, —(C₃₋₈ cycloalkylene —C₁₋₁₀ alkylene-, 4 to 14-membered heterocycloalkylene, —C₁₋₁₀ alkylene-(4 to 14-membered heterocycloalkylene)-, -(4 to 14-membered heterocycloalkylene)-C₁₋₁₀ alkylene-, —C₁₋₁₀ alkylene-C(═O)—, —C₁₋₁₀ heteroalkylene-C(═O)—, —C₃₋₈ cycloalkylene-C(═O)—, —O—(C₁₋₈ alkyl)-C(═O)—, -arylene-C(═O)—, —C₁₋₁₀ alkylene-arylene-C(═O)—, -arylene —C₁₋₁₀ alkylene-C(═O)—, —C₁₋₁₀ alkylene-(C₃₋₈ cycloalkylene)-C(═O)—, —(C₃₋₈ cycloalkylene)-C₁₋₁₀ alkylene-C(═O)—, -4 to 14-membered heterocycloalkylene-C(═O)—, —C₁₋₁₀ alkylene-(4 to 14-membered heterocycloalkylene)-C(═O)—, -(4 to 14-membered heterocycloalkylene)-C₁₋₁₀ alkylene-C(═O)—, —C₁₋₁₀ alkylene-NH—, —C₁₋₁₀ heteroalkylene-NH—, —C₃₋₈ cycloalkylene-NH—, —O—(C₁₋₈ alkyl)-NH—, -arylene-NH—, —C₁₋₁₀ alkylene-arylene-NH—, -arylene-C₁₋₁₀ alkylene-NH—, —C₁₋₁₀ alkylene-(C₃-s cycloalkylene)-NH—, —(C₃₋₈ cycloalkylene)-C₁₋₁₀ alkylene-NH—, -4 to 14-membered heterocycloalkylene-NH—, —C₁₋₁₀ alkylene-(4 to 14-membered heterocycloalkylene)-NH—, -(4 to 14-membered heterocycloalkylene)-C₁₋₁₀ alkylene-NH—, —C₁₋₁₀ alkylene-S—, —C₁₋₁₀ heteroalkylene-S—, —C₃₋₈ cycloalkylene-S—, —O—C₁₋₈ alkyl)-S—, -arylene-S—, —C₁₋₁₀ alkylene-arylene-S—, -arylene-C₁₋₁₀ alkylene-S—, —C₁₋₁₀ alkylene-(C₃₋₈ cycloalkylene)-S—, —(C₃-s cycloalkylene)-C₁₋₁₀ alkylene-S—, -4 to 14-membered heterocycloalkylene-S—, —C₁₋₁₀ alkylene-(4 to 14-membered heterocycloalkylene)-S—, or -(4 to 14-membered heterocycloalkylene)-C₁-C₁₀ alkylene-S—;
each Z₅ independently is absent, R₅₇-R₁₇ or a polyether unit,
each R₅₇ independently is a bond, NR₂₃, S or O;
each R₂₃ independently is hydrogen, C₁₋₆ alkyl, C₆₋₁₀ aryl, C₃₋₈ cycloalkyl, —COOH, or —COO—C₁₋₆ alkyl; and
each Z₆ independently is absent, —C₁₋₁₀ alkyl-R₃—, —C₁₋₁₀ alkyl-NR₅—, —C₁₋₁₀ alkyl-C(O)—, —C₁₋₁₀ alkyl-O—, —C₁₋₁₀ alkyl-S— or —(C₁₋₁₀ alkyl-R₃)_{g1}—C₁₋₁₀ alkyl-C(O)—;
each R₃ independently is —C(O)—NR₅— or —NR₅—C(O)—;
each R₅ independently is hydrogen, C₁₋₆ alkyl, C₆₋₁₀ aryl, C₃₋₈ cycloalkyl, COOH, or COO—C₁₋₆ alkyl; and
g₁ is an integer from 1 to 4.

11. The conjugate of any one of the preceding claims, wherein Z₄ is 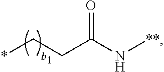
in which b₁ is 1 or 4.

12. The conjugate of any one of the preceding claims, wherein Z₄ is 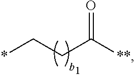
in which b₁ is 1 or 4.

13. The conjugate of any one of the preceding claims, wherein Z₄ is

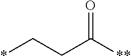

14. The conjugate of any one of the preceding claims, wherein Z₄ is

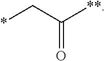

15. The conjugate of any one of the preceding claims, wherein each Z₅ independently is a polyalkylene glycol (PAO).

16. The conjugate of any one of the preceding claims, wherein M^{P}, when present, is 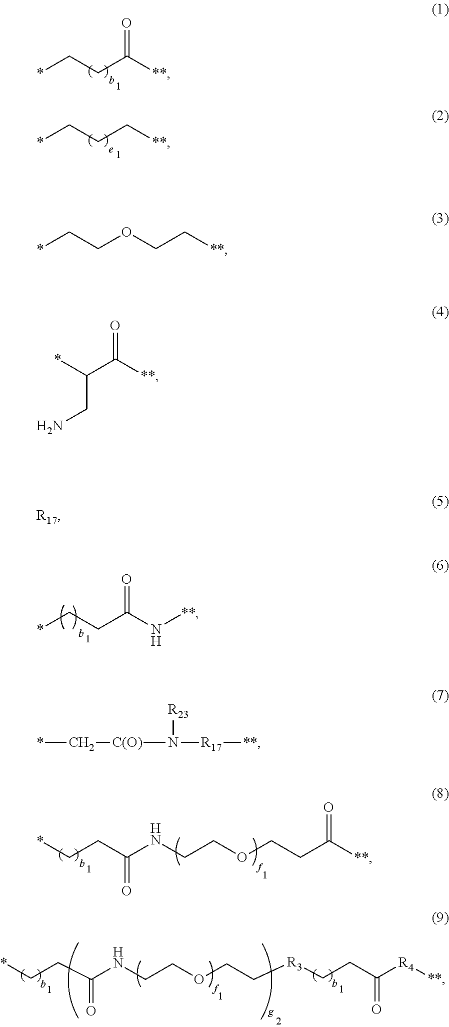 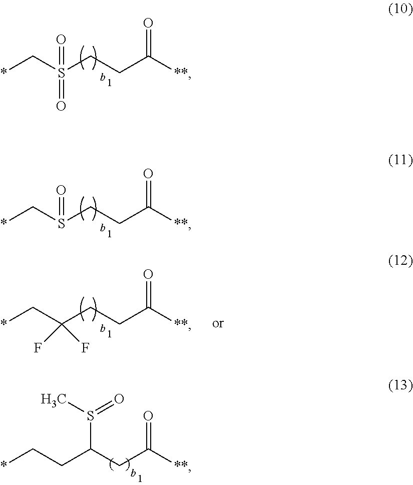
wherein * denotes attachment to L^{P′} or L^{P} and ** denotes attachment to L^{M};
R₃, R₅, R₁₇, and R₂₃ are as defined herein;
R₄ is a bond or —NR₅—(CR₂₀R₂₁)—C(O)—:
each R₂₀ and R₂₁ independently is hydrogen, C₁₋₆ alkyl, C₆₋₁₀ aryl, hydroxylated C₆₋₁₀ aryl, polyhydroxylated C₆₋₁₀ aryl, 5 to 12-membered heterocycle, C₃₋₈ cycloalkyl, hydroxylated C₃₋₈ cycloalkyl, polyhydroxylated C₃₋₈ cycloalkyl or a side chain of a natural or unnatural amino acid;
each b₁ independently is an integer from 0 to 6;
e₁ is an integer from 0 to 8,
each f₁ independently is an integer from 1 to 6; and
g₂ is an integer from 1 to 4.

17. The conjugate of any one of the preceding claims, wherein M^{P}, when present, is: 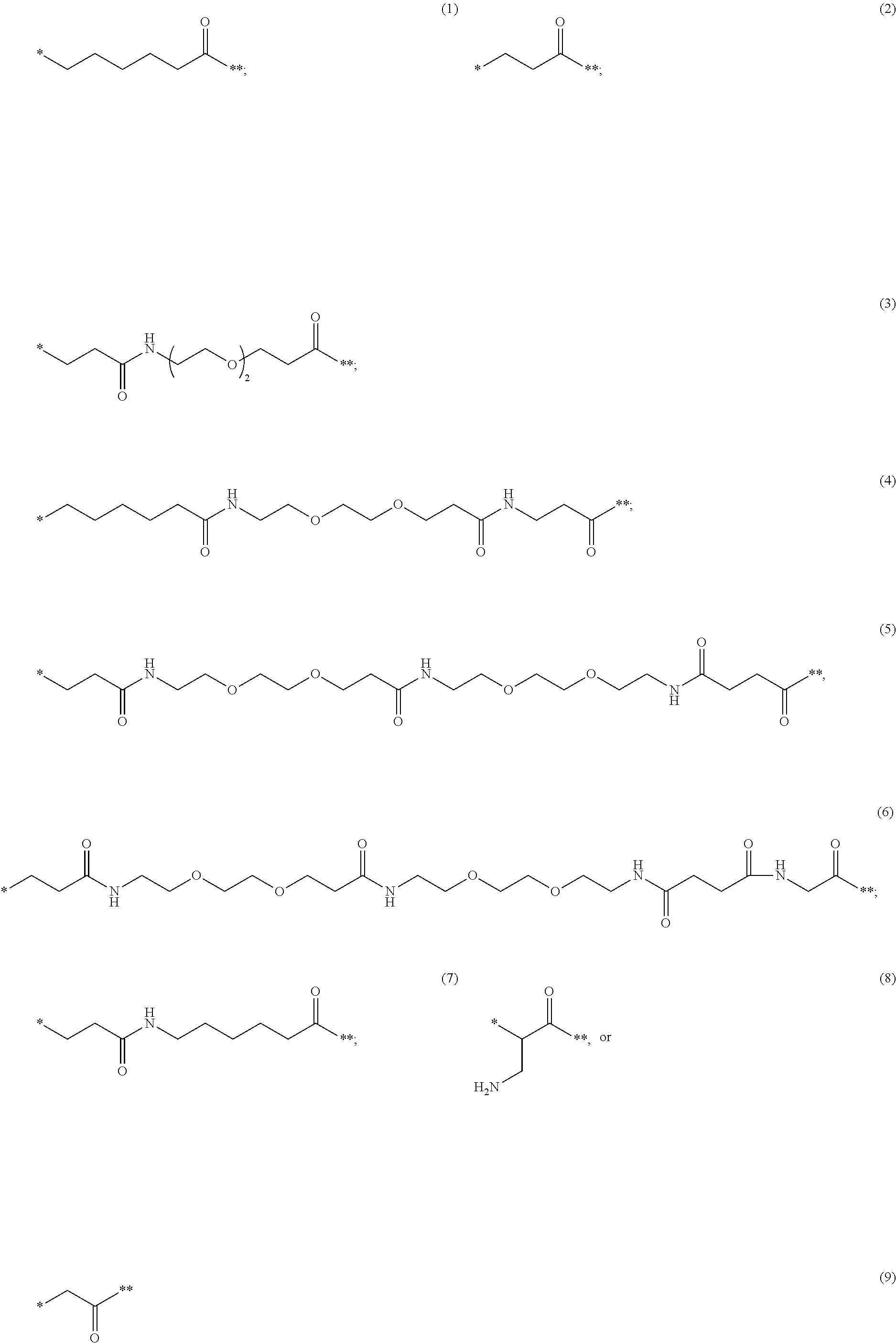
wherein * denotes attachment to L^{P′} or L^{P} and ** denotes attachment to L^{M}.

18. The conjugate of any one of the preceding claims, wherein M^{P}, when present, is: 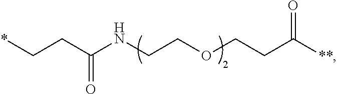
wherein * denotes attachment to L^{P′} or L^{P} and ** denotes attachment to M^{A}.

19. The conjugate of any one of the preceding claims, wherein M^{A} comprises a peptide moiety of at least two amino acid (AA) units.

20. The conjugate or scaffold of any one of the preceding claims, wherein M^{A} comprises a peptide moiety that contains at least about five amino acids.

21. The conjugate or scaffold of any one of the preceding claims, wherein M^{A} comprises a peptide moiety that contains at most about ten amino acids.

22. The conjugate or scaffold of any one of the preceding claims, wherein M^{A} comprises a peptide moiety that contains from three to about ten amino acids selected from glycine, serine, glutamic acid, aspartic acid, lysine, cysteine and a combination thereof.

23. The conjugate or scaffold of any one of the preceding claims, wherein M^{A} comprises a peptide moiety that contains at least four glycines and at least one serine.

24. The conjugate or scaffold of any one of the preceding claims, wherein M^{A} comprises a peptide moiety that contains at least four glycines and at least one glutamic acid.

25. The conjugate or scaffold of any one of the preceding claims, wherein M^{A} comprises a peptide moiety that contains at least four glycines, at least one serine and at least one glutamic acid.

26. The conjugate of any one of the preceding claims, wherein L^{D} comprises a peptide of 1 to 12 amino acids, wherein each amino acid is independently selected from alanine, β-alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, selenocysteine, ornithine, penicillamine, aminoalkanoic acid, aminoalkynoic acid, aminoalkanedioic acid, aminobenzoic acid, amino-heterocyclo-alkanoic acid, heterocyclo-carboxylic acid, citrulline, statine, diaminoalkanoic acid, and derivatives thereof.

27. The conjugate of any one of the preceding claims, wherein L^{D} comprises β-alanine.

28. The conjugate of any one of the preceding claims, wherein L^{D} comprises (β-alanine)-(alanine)-(alanine) or (β-alanine)-(valine)-(alanine).

29. The conjugate of any one of the preceding claims, wherein the hydrophilic group comprises a polyalcohol or a derivative thereof, a polyether or a derivative thereof, or a combination thereof.

30. The conjugate of any one of the preceding claims, wherein the hydrophilic group comprises an amino polyalcohol.

31. The conjugate of any one of the preceding claims, wherein T′ comprises one or more of the following fragments of the formula: 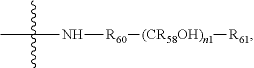
in which
n₁ is an integer from 0 to about 6;
each R₅₈ is independently hydrogen or C₁₋₈ alkyl;
R₆₀ is a bond, a C₁₋₆ alkyl linker, or —CHR₅₉— in which R₅₉ is H, alkyl, cycloalkyl, or arylalkyl;
R₆₁ is CH₂OR₆₂, COOR₆₂, —(CH₂)ₙ₂COOR₆₂, or a heterocycloalkyl substituted with one or more hydroxyl;
R₆₂ is H or C₁₋₈ alkyl; and
n₂ is an integer from 1 to about 5.

32. The conjugate of any one of the preceding claims, wherein T′ comprises glucamine.

33. The conjugate of any one of the preceding claims, wherein T′ comprises:

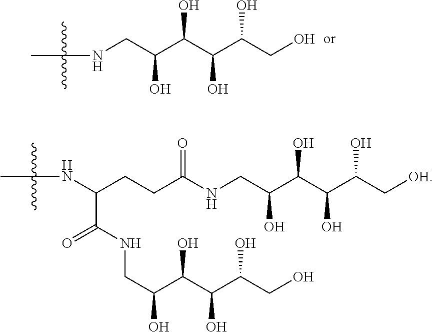 [SMILES: *NC[C@H](O)[C@@H](O)[C@H](O)[C@H](O)CO ; *NC(CCC(=O)NC[C@H](O)[C@@H](O)[C@H](O)[C@H](O)CO)C(=O)NC[C@H](O)[C@@H](O)[C@H](O)[C@H](O)CO]

34. The conjugate of any one of the preceding claims, wherein T′ comprises 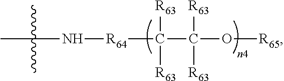
in which
n₄ is an integer from 1 to about 25;
each R₆₃ is independently hydrogen or C₁₋₈ alkyl;
R₆₄ is a bond or a C₁₋₈ alkyl linker;
R₆₅ is H, C₁₋₈ alkyl, —(CH₂)ₙ₂COOR₆₂ or —(CH₂)ₙ₂COR₆₆;
R₆₂ is H or C₁₋₈ alkyl;
R₆₆ is 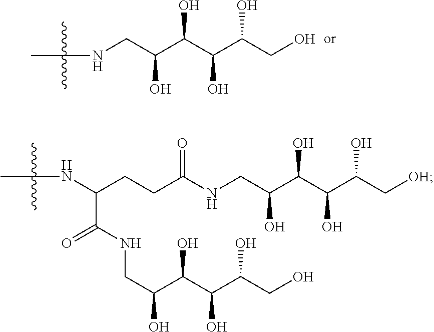 [SMILES: *NC[C@H](O)[C@@H](O)[C@H](O)[C@H](O)CO ; *NC(CCC(=O)NC[C@H](O)[C@@H](O)[C@H](O)[C@H](O)CO)C(=O)NC[C@H](O)[C@@H](O)[C@H](O)[C@H](O)CO]
and
n₂ is an integer from 1 to about 5.

35. The conjugate of any one of the preceding claims, wherein T′ comprises: 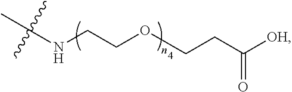
in which n₄ is an integer from about 2 to about 20, from about 4 to about 16, from about 6 to about 12, from about 8 to about 12.

36. The conjugate of any one of the preceding claims, wherein T′ comprises: 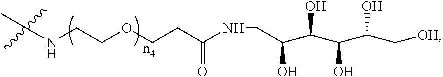
in which n₄ is an integer from about 2 to about 20, from about 4 to about 16, from about 6 to about 12, from about 8 to about 12.

37. The conjugate of any one of the preceding claims, wherein T′ comprises: 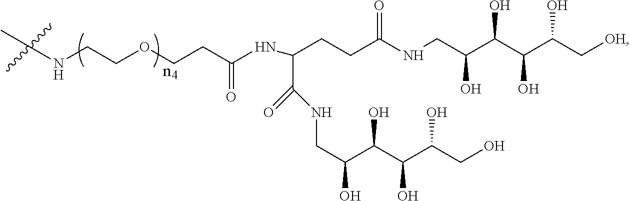
in which n₄ is an integer from about 2 to about 20, from about 4 to about 16, from about 6 to about 12, from about 8 to about 12.

38. The conjugate of any one of the preceding claims, wherein n₄ is 6, 7, 8, 9, 10, 11, or 12.

39. The conjugate of any one of the preceding claims, wherein n₄ is 8 or 12.

40. The conjugate of claim 1, being of Formula (III):
PBRM-(A¹ₐ₆-L¹ₛ₂-L²_{y1}-D)_{d13} (III)
or pharmaceutically acceptable salt or solvate thereof, wherein:
PBRM denotes a protein based recognition-molecule;
each occurrence of D is independently a PBD drug moiety;
A¹ is a stretcher unit;
a₆ is an integer 1 or 2;
L¹ is a specificity unit;
s₂ is an integer from about 0 to about 12;
L² is a spacer unit;
y1 is an integer from 0 to 2; and
d₁₃ is an integer from about 1 to about 14.

41. The conjugate of any one of the preceding claims, being of any one of Formulae (IIIa) to (IIIf): 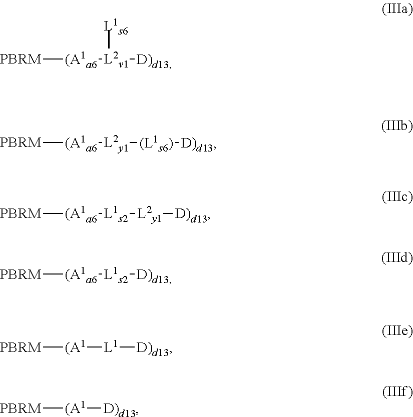
or a pharmaceutically acceptable salt or solvate thereof, wherein:
PBRM denotes a protein based recognition-molecule;
each occurrence of D is independently a PBD drug moiety;
A¹ is a stretcher unit linked to the spacer unit L²;
a₆ is an integer 1 or 2;
L¹ is a specificity unit linked to the spacer unit L²;
s₂ is an integer from about 0 to about 12;
s₆ is an integer from about 0 to about 12;
L² is a spacer unit;
y₁ is an integer 0, 1 or 2; and
d₁₃ is an integer from about 1 to about 14.

42. The conjugate of any one of the preceding claims, wherein the PBD drug moiety (I)) is of Formula (IV), 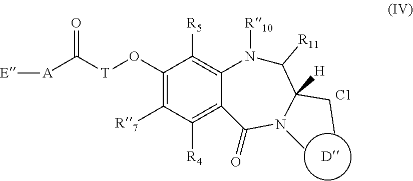
a tautomer thereof, a pharmaceutically acceptable salt or solvate thereof, or a pharmaceutically acceptable salt or solvate of the tautomer, wherein:
E″ is a direct or indirect linkage to the PBRM (e.g., antibody or antibody fragment), E, or 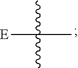
in which 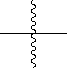
denotes direct or indirect linkage to the PBRM via a functional group of E;
D″ is D′ or 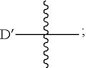
in which 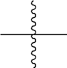
denotes direct or indirect linkage to the PBRM via a functional group of D′;
R″₇ is a direct or indirect linkage to the PBRM (e.g., antibody or antibody fragment), R₇, or 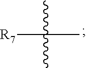
in which 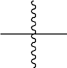
denotes direct or indirect linkage to the PBRM via a functional group of R₇;
R″₁₀ is a direct or indirect linkage to the PBRM, R₁₀, or 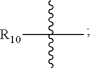
in which 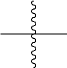
denotes direct or indirect linkage the PBRM via a functional group of R₁₀; and
wherein the PBD drug moiety (D) is directly or indirectly linked to the PBRM antibody or antibody fragment) via a functional group of one of E″, D″, R″₇, and R″₁₀.

43. The conjugate of any one of the preceding claims, wherein E″ is a direct or indirect linkage to L^{C}, E, or 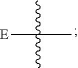
in which 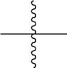
denotes direct or indirect linkage to L^{C} via a functional group of E.

44. The conjugate of any one of the preceding claims, wherein E″ is a direct or indirect linkage to L^{D}, E, or 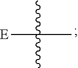
in which 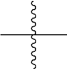
denotes direct or indirect linkage to L^{D} via a functional group of E.

45. The conjugate of any one of the preceding claims, wherein D″ is D′ or 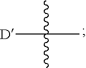
in which 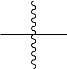
denotes direct or indirect linkage to L^{C} via a functional group of D′.

46. The conjugate of any one of the preceding claims, wherein D″ is D′ or 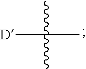
in which 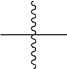
denotes direct or indirect linkage to L^{D} via a functional group of D′.

47. The conjugate of any one of the preceding claims, wherein R″₇ is a direct or indirect linkage to L^{C}, R₇ or 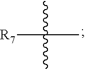
in which 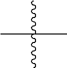
denotes direct or indirect linkage to L^{C} via a functional group of R₇.

48. The conjugate of any one of the preceding claims, wherein R″₇ is a direct or indirect linkage to L^{D}, R₇ or 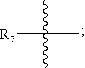
in denotes 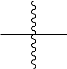
direct or indirect linkage to L^{D} via a functional group of R₇.

49. The conjugate of any one of the preceding claims, wherein R″₁₀ is a direct or indirect linkage to L^{C}, R₁₀, or 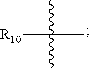
in which 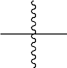
denotes direct or indirect linkage L^{C} via a functional group of R₁₀.

50. The conjugate of any one of the preceding claims, wherein R″₁₀ is a direct or indirect linkage to L^{D}, R₁₀, or 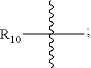
in which 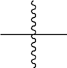
denotes direct or indirect linkage L^{C} via a functional group of R₁₀.

51. The conjugate of any one of the preceding claims, wherein E is a direct or indirect linkage to the PBRM; D″ is D′; R″₇₇ is R₇ and R″₁₀ is R₁₀.

52. The conjugate of any one of the preceding claims, wherein E″ is a direct or indirect linkage to L^{C}; D″ is D′; R″₇ is R₇ and R″₁₀ is R₁₀.

53. The conjugate of any one of the preceding claims, wherein E″ is a direct or indirect linkage to L^{D}; D″ is D′; R″₇ is R₇ and R″₁₀ is R₁₀.

54. The conjugate of any one of the preceding claims, wherein E″ is 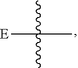
in which 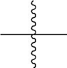
denotes direct or indirect linkage to the PBRM via a functional group of E; D″ is D′; R″₇ is R₇; and R″₁₀ is R₁₀.

55. The conjugate of any one of the preceding claims, wherein E″ is 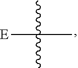
in which 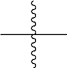
denotes direct or indirect linkage to L^{C} via a functional group of E; D″ is D′; R″₇ is R₇; and R″₁₀ is R₁₀.

56. The conjugate of any one of the preceding claims, wherein E″ is 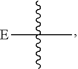
in which 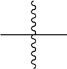
denotes direct or indirect linkage to L^{D} via a functional group of E; D″ is D′; R″₇ is R₇; and R″₁₀ is R₁₀.

57. The conjugate of any one of the preceding claims, wherein D″ is 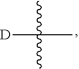 [SMILES: *[2H]]
in which 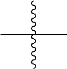
denotes direct or indirect linkage to the PBRM via a functional group of D; E″ is E, R″₇ is R₇; and R″₁₀ is R₁₀.

58. The conjugate of any one of the preceding claims, wherein D″ is 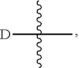 [SMILES: *[2H]]
in which 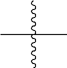
denotes direct or indirect linkage to L^{C} via a functional group of D; E″ is E; R″₇ is R₇; and R″₁₀ is R₁₀.

59. The conjugate of any one of the preceding claims, wherein D″ is 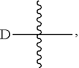 [SMILES: *[2H]]
in which 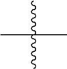
denotes direct or indirect linkage to L^{D} via a functional group of D; E″ is E; R″₇ is R₇; and R″₁₀ is R₁₀.

60. The conjugate of any one of the preceding claims, wherein R″₇ is a direct or indirect linkage to the PBRM; E″ is E; D″ is D′; and R″₁₀ is R₁₀.

61. The conjugate of any one of the preceding claims, wherein R″₇ is a direct or indirect linkage to L^{C}; E″ is E; D″ is D′; and R″₁₀ is R₁₀.

62. The conjugate of any one of the preceding claims, wherein R″₇ is a direct or indirect linkage to L^{D}; E″ is E; D″ is D′; and R″₁₀ is R₁₀.

63. The conjugate of any one of the preceding claims, wherein R″₇ is 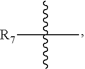
in which 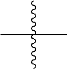
denotes direct or indirect linkage to the PBRM via a functional group of R₇; E″ is E; D″ is D′; and R″₁₀ is R₁₀.

64. The conjugate of any one of the preceding claims, wherein R″₇ is 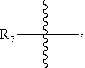
in which 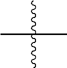
denotes direct or indirect linkage to L^{C} via a functional group of R₇; E″ is E; D″ is D′; and R″₁₀ is R₁₀.

65. The conjugate of any one of the preceding claims, wherein R″₇ is 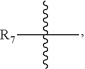
in which 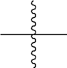
denotes direct or indirect linkage to L^{D} via a functional group of R₇; E″ is E; D″ is D′; and R″₁₀ is R₁₀.

66. The conjugate of any one of the preceding claims, wherein R″₁₀ is a direct or indirect linkage to the PBRM; E″ is E; D″ is D′; and R″₇ is R₇.

67. The conjugate of any one of the preceding claims, wherein R″m is a direct or indirect linkage to L^{C}; E″ is E; D″ is D′; and R″₇ is R₇.

68. The conjugate of any one of the preceding claims, wherein R″m is a direct or indirect linkage to L^{D}; E″ is E; D″ is D′; and R″₇ is R₇. The conjugate of any one of the preceding claims, wherein R″₁₀ is 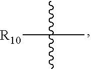
in which 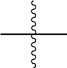
denotes direct or indirect linkage to the PBRM via a functional group of R₁₀; E″ is E; D″ is D′; and R″₇ is R₇.

69. The conjugate of any one of the preceding claims, wherein R″₁₀ is 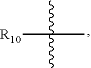
in which 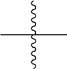
denotes direct or indirect linkage to L^{C} via a functional group of R₁₀; E″ is E; D″ is D′; and R″₇ is R₇.

70. The conjugate of any one of the preceding claims, wherein R″₁₀ is 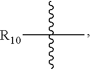
in which 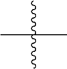
denotes direct or indirect linkage to L^{D} via a functional group of R₁₀; E″ is E; D″ is D′; and R″₇ is R₇.

71. The conjugate of any one of the preceding claims, wherein:
D′ is D1, D2, D3, or D4: 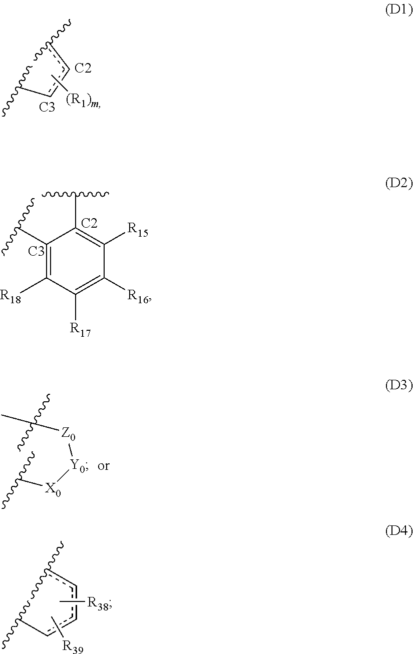
wherein the dotted line between C2 and C3 or between C2 and C1 in D1 or the dotted line in D4 indicates the presence of a single or double bond; and
m is 0, 1 or 2;
when D′ is D1, the dotted line between C2 and C3 is a double bond, and m is 1, then R₁ is:
(i) C₆₋₁₀ aryl group, optionally substituted by one or more substituents selected from —OH, halo, —NO₂, —CN, —N₃, —OR₂, —COON, —COOR₂, —COR₂, —OCONRF₁₃R₁₄, C₁₋₁₀ alkyl, C₃₋₁₀ cycloalkyl, C₂₋₁₀ alkenyl, C₂₋₁₀ alkenyl, a polyethylene glycol unit —(OCH₂CH₂)ᵣ—OR^{a}, 3- to 14-membered heterocycloalkyl, 5- to 12-membered heteroaryl, bis-oxy-C₁₋₃ alkylene, —NR₁₃R₁₄, —S(═O)₂R₁₂, —S(═O)₂NR₁₃R₁₄, —SOₓM, —OSOₓM, —NR₉COR₁₉, —NH(C═NH)NH₂;
(ii) C₁₋₅ alkyl;
(iii) C₃₋₆ cycloalkyl;
(iv) 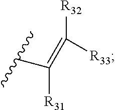
(vi) 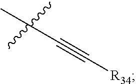
(vii) 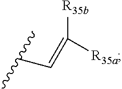
(viii) 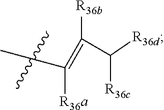
or
(viii) halo;
when D′ is D1, the dotted line between C2 and C3 is a single bond, and m is 1, then R₁ is:
(i) —OH, ═O, ═CH₂, —CN, —R₂, —OR₂, halo, ═CH—R₆, ═C(R₆)₂, —O—SO₂R₂,—CO₂R₂, —COR₂, —CHO, or —COON; or
(ii) 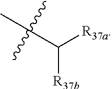
when D′ is D1 and m is 2, then each R₁ independently is halo and either both R₁ are attached to the same carbon atom or one is attached to C2 and the other is attached to C3;
T is C₁₋₁₀ alkylene linker;
A is 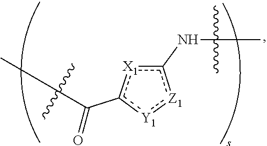
wherein the —NH group of A is connected to the —C(O)-T- moiety of Formula (IV) and the C═O moiety of A is connected to E; and each 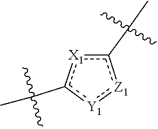
independently is 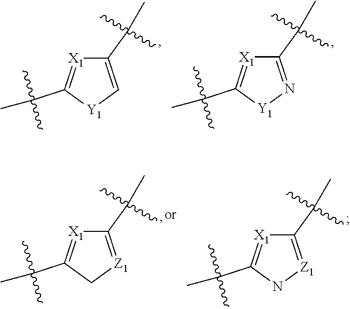
E is E1, E2, E3, E4, E5 or E6: 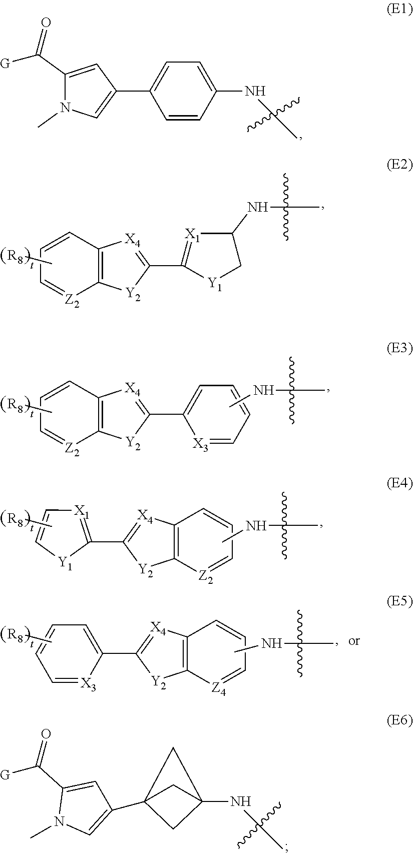
G is G1, G2, G3, G4, OH, —NH—(C₁₋₆ alkylene)-R₁₃ₐ, —NR₁₃R₁₄, O—(CH₂)₃—NH₂, —O—CH(CH₃)—(CH₂)₂—NH₂ or —NH—(CH₂)₃—O—C(═O)—CH(CH₃)—NH₂: 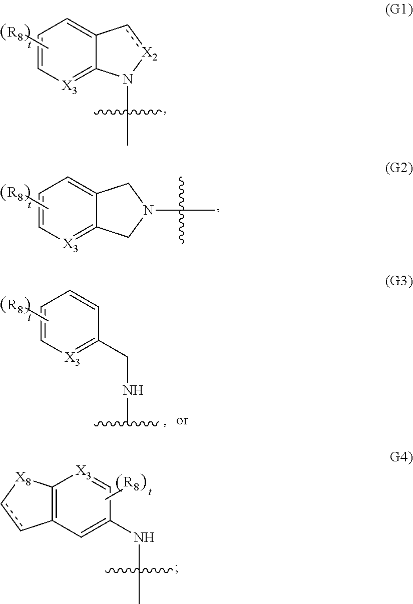
wherein the dotted line in G1 or G4 indicates the presence of a single or double bond;
each occurrence of R₂ and R₃ independently is an optionally substituted CA-s alkyl, optionally substituted C₂₋₈ alkenyl, optionally substituted C₂₋₈ alkynyl, optionally substituted C₃₋₈ cycloalkyl, optionally substituted 3- to 20-membered heterocycloalkyl, optionally substituted C₆₋₂₀ aryl or optionally substituted 5- to 20-membered heteroaryl, and, optionally in relation to the group NR₂R₃, R₂ and R₃ together with the nitrogen atom to which they are attached form an optionally substituted 4-, 5-, 6- or 7-membered heterocycloalkyl or an optionally substituted 5- or 6-membered heteroaryl;
R₄, R₅ and R₇ are each independently —H, —R₂, —OH, —OR₂, —SH, —SR₂, —NH₂, —NHR₂, —NR₂R₃, —NO₂, —SnMe₃, halo or a polyethylene glycol unit —(OCH₂CH₂)ᵣ—OR^{a}; or R₄ and R₇ together form bis-oxy-C₁₋₃ alkylene;
each R₆ independently is —H, —R₂, —CO₂R₂, —COR₂, —CHO, —CO₂H, or halo;
each R₈ independently is —OH, halo, —NO₂, —CN, —N₃, —OR₂, —COOH, —COOR₂, —COR₂, —OCONR₁₃R₁₄, —CONR₁₃R₁₄, —CO—NH—(C₁₋₆ alkylene)-R₁₃ₐ, C₁₋₁₀ alkyl, C₃₋₁₀ cycloalkyl, C₂₋₁₀ alkenyl, C₂₋₁₀ alkynyl, a polyethylene glycol unit —(OCH₂CH₂)ᵣ—OR^{a}, 3- to 14-membered heterocycloalkyl, 5- to 12-membered heteroaryl, —S(═O)₂R₁₂, —S(═O)₂NR₁₃R₁₄, —SOₓM, —OSOₓM, —NR₉COR₁₉, —NH(C═NH)NH₂, —R₂₀-R₂₁—NR₁₃R₁₄, —R₂₀-R₂₁—NH—P(O)(OH)—(OCH₂CH₂)ₙ₉—OCH₃, or —O—P(O)(OH)—(OCH₂CH₂)ₙ₉—OCH₃;
each R₉ independently is C₁₋₁₀ alkyl, C₃₋₁₀ cycloalkyl, C₂₋₁₀ alkenyl or C₂₋₁₀ alkynyl;
R¹⁰ is —H or a nitrogen protecting group;
R₁₁ is —OR^{Q} or —SOₓM;
or R¹⁰ and R¹¹ taken together with the nitrogen atom and carbon atom to which they are respectively attached, form a N═C double bond;
each R₁₂ independently is C₁₋₇ alkyl, 3- to 20-membered heterocycloalkyl, 5- to 20-membered heteroaryl, or C₆₋₂₀ aryl;
each occurrence of R₁₃ and R₁₄ are each independently H, C₁₋₁₀ alkyl, 3- to 20-membered heterocycloalkyl, 5- to 20-membered heteroaryl, or C₆₋₂₀ aryl;
each R₁₃ₐ independently is —OH or —NR₁₃R₁₄;
R₁₅, R₁₆, R₁₇ and R₁₈ are each independently —H, —OH, halo, —NO₂, —CN, —N₃, —OR₂, —COOH, —COOR₂, —COR₂, —OCONR₁₃R₁₄, C₁₋₁₀ alkyl, C₃₋₁₀ cycloalkyl, C₂₋₁₀ alkenyl, C₂₋₁₀ alkynyl, a polyethylene glycol unit —(OCH₂CH₂)ᵣ—ORₐ, 3-14 membered heterocycloalkyl, 5- to 12-membered heteroaryl, —NR₁₃R₁₄, —S(═O)₂R₁₂, —S(═O)₂NR₁₃R₁₄, —SR₁₂, —SOₓM, —OSOₓM, —NR₉COR₁₉ or —NH(C═NH)NH₂;
each R₁₉ independently is C₁₋₁₀ alkyl, C₃₋₁₀ cycloalkyl, C₂₋₁₀ alkenyl or C₂₋₁₀ alkynyl;
each R₂₀ independently is a bond, C₆₋₁₀ arylene, 3-14 membered heterocycloalkylene or 5- to 12-membered heteroarylene;
each R₂₁ independently is a bond or C₁₋₁₀ alkylene;
R₃₁, R₃₂ and R₃₃ are each independently —H, C₁₋₃ alkyl, C₂₋₃ alkenyl, C₂₋₃ alkynyl or cyclopropyl, wherein the total number of carbon atoms in the R₁ group is no more than 5;
R₃₄ is —H, C₁₋₃ alkyl, C₂₋₃ alkenyl, C₂₋₃ alkynyl, cyclopropyl, or phenyl wherein the phenyl is optionally substituted by one or more of halo, methyl, methoxy, pyridyl or thiophenyl;
one of R₃₅ₐ and R_{35b} is —H and the other is a phenyl group optionally substituted with one or more of halo, methyl, methoxy, pyridyl or thiophenyl;
R₃₆ₐ, R_{36b}, R_{36c} are each independently —H or C₁₋₂ alkyl;
R_{36d} is —OH, —SH, —COOH, —C(O)H, —N═C═O, —CONHNH₂, 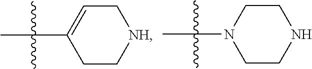 [SMILES: *C1=CCNCC1 ; *N1CCNCC1]
or, wherein NHR^{N}, is or C₁₋₄ alkyl;
R₃₇ₐ and R_{37b} are each independently is —H, —F, C₁₋₄ alkyl, C₂₋₃ alkenyl, wherein the alkyl and alkenyl groups are optionally substituted by C₁₋₄ alkyl amido or C₁₋₄ alkyl ester; or when one of R₃₇ₐ and R_{37b} is —H, the other is —CN or a C₁₋₄ alkyl ester;
R₃₈ and R₃₉ are each independently H, R₁₃, ═CH₂, =CH—(CH₂)ₛ₁—CH₃, ═O, (CH₂)ₛ₁—OR₁₃, (CH₂)ₛ₁—CO₂R₁₃, (CH₂)ₛ₁—NR₁₃R₁₄, O—(CH₂)₂—NR₁₃R₁₄, NH—C(O)—R₁₃, O—(CH₂)s-NH—C(O)—R₁₃, O—(CH₂)s-C(O)NHR₁₃, (CH₂)ₛ₁0S(═O)₂R₁₃, O—SO₂R₁₃, (CH₂)ₛ₁—C(O)R₁₃ and (CH₂)ₛ₁—C(O)NR₁₃R₁₄;
X₀ is CH₂, NR₆, C═O, BH, SO or SO₂;
Y₀ is O, CH₂, NR₆ or S;
Z₀ is absent or (CH₂)ₙ;
each X₁ independently is CR_{b}, or N;
each Y₁ independently is CH, NRₐ, O or S;
each Z₁ independently is CH, NRₐ, O or S;
each Rₐ independently is H or C₁₋₄ alkyl;
each R_{b} independently is H, OH, C₁₋₄ alkyl, or C₁₋₄ alkoxyl;
X₂ is CH, CH₂ or N;
X₃ is CH or N;
X₄ is NH, O or S;
X₈ is NH, O or S,
Q is O, S or NH;
when Q is S or NH, then R^{Q} is —H or optionally substituted C₁₋₂ alkyl; or
when Q is O, then R^{Q} is —H or optionally substituted C₁₋₂ alkyl, —SOₓM, —PO₃M, —(CH₂—CH₂—)ₙ₉CH₃, —(CH₂—CH₂O)ₙ₉—(CH₂)₂—R₄₀, —C(O)—(CH₂—CH₂—O)ₙ₉CH₃, —C(O)O—(CH²—CH₂—O)ₙ₉CH₃, —C(O)NH—)—(CH₂—CH₂—O)ₙ₉CH₃, —(CH₂)ₙ—NH—C(O)—CH₂—O—CH₂—C(O)—NH—(CH₂—CH₂ —O)ₙ₉CH₃, —(CH₂)ₙ—NH—C(O)—(CH₂)ₙ—(CH₂—CH₂—O)ₙ₉CH₃, a sugar moiety, 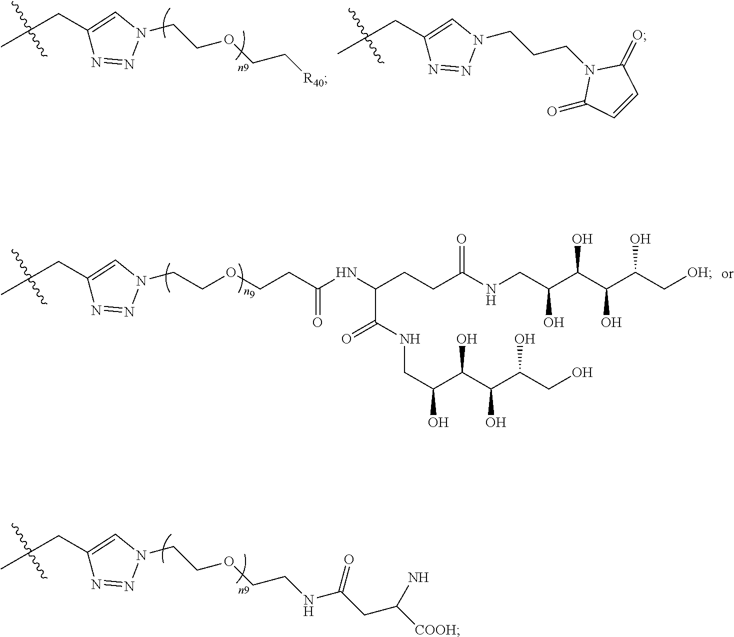
each M independently is H or a monovalent pharmaceutically acceptable cation;
n is 1, 2 or 3;
n₉ is 1, 2, 3, 4, 5, 6, 8, 12 or 24;
each r independently is an integer from 1 to 200;
s is 1, 2, 3, 4, 5 or 6;
s₁ is 0, 1, 2, 3, 4, 5 or 6;
t is 0, 1, or 2;
R₄₀ is —SO₃H, —COON, —C(O)NH(CH₂)₂SO₃H, or —C(O)NH(CH₂)₂COOH; and
each x independently is 2 or 3.

72. The conjugate of any one of the preceding claims, wherein the PBD drug moiety (D) is of Formula (IV-a), 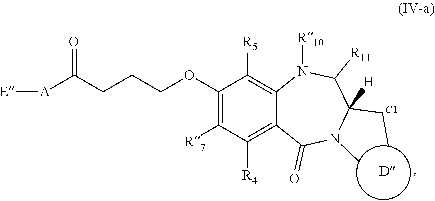
a tautomer thereof, a pharmaceutically acceptable salt or solvate thereof, or a pharmaceutically acceptable salt or solvate of the tautomer.

73. The conjugate of any one of the preceding claims, wherein D′ is D1.

74. The conjugate of any one of the preceding claims, wherein the PBD drug moiety (D) is of any one of formulae (V-1), (V-2), and (V-3): 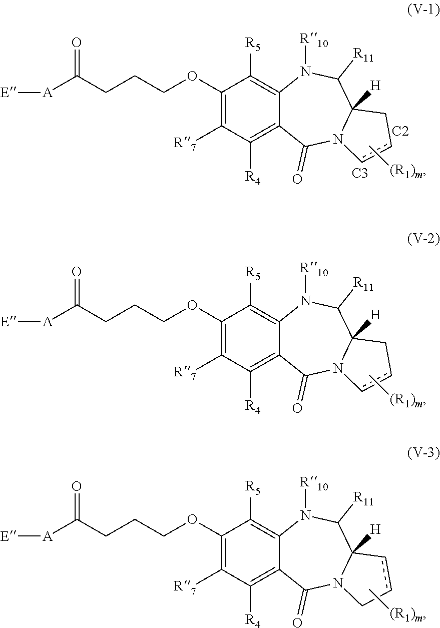
a tautomer thereof, a pharmaceutically acceptable salt or solvate thereof, or a pharmaceutically acceptable salt or solvate of the tautomer.

75. The conjugate of any one of the preceding claims, wherein the PBD drug moiety (D) is of Formula (VI-1): 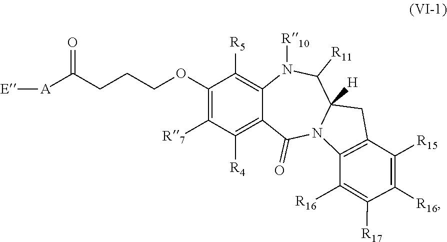
a tautomer thereof, a pharmaceutically acceptable salt or solvate thereof, or a pharmaceutically acceptable salt or solvate of the tautomer.

76. The conjugate of any one of the preceding claims, whereinthe PBD drug moiety (D) is of Formula (VII), (VII-1), (VII-2) or (VII-3): 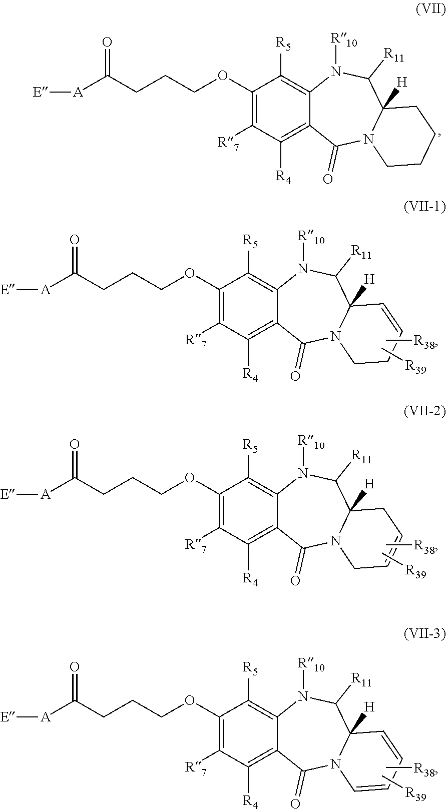
a tautomer thereof, a pharmaceutically acceptable salt or solvate thereof, or a pharmaceutically acceptable salt or solvate of the tautomer.

77. The conjugate of any one of the preceding claims, wherein the PBD drug moiety (D) is of Formula (VIII): 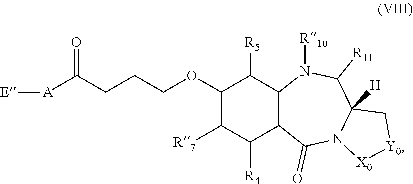
a tautomer thereof, a pharmaceutically acceptable salt or solvate thereof, or a pharmaceutically acceptable salt or solvate of the tautomer.

78. The conjugate of any one of the preceding claims, wherein T is C₂₋₄ alkylene linker.

79. The conjugate of any one of the preceding claims, wherein A is

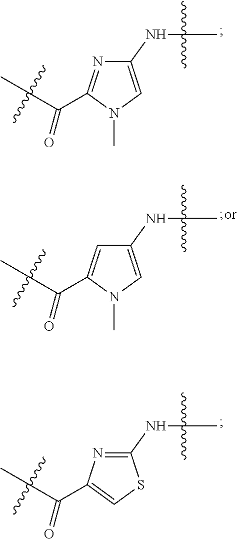 [SMILES: *Nc1cn(C)c(C(*)=O)n1 ; *Nc1cc(C(*)=O)n(C)c1 ; *Nc1nc(C(*)=O)cs1]

80. The conjugate of any one of the preceding claims, wherein A is 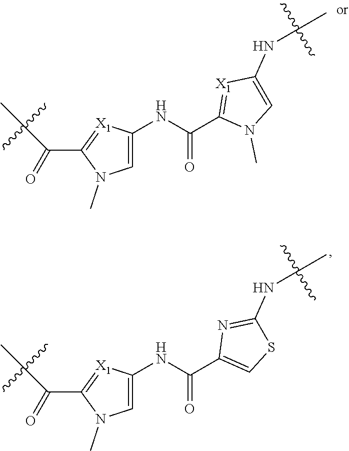
wherein each X₁ independently is CH or N.

81. The conjugate of any one of the preceding claims, wherein A is 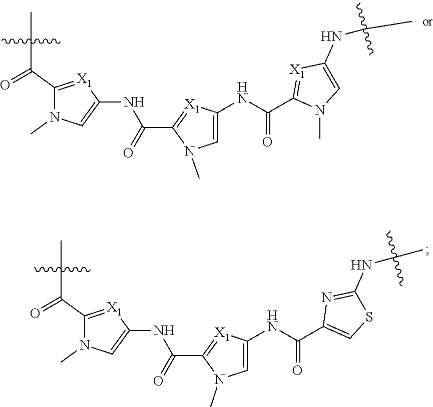
wherein each X₁ independently is CH or N.

82. The conjugate of any one of the preceding claims, wherein A is: 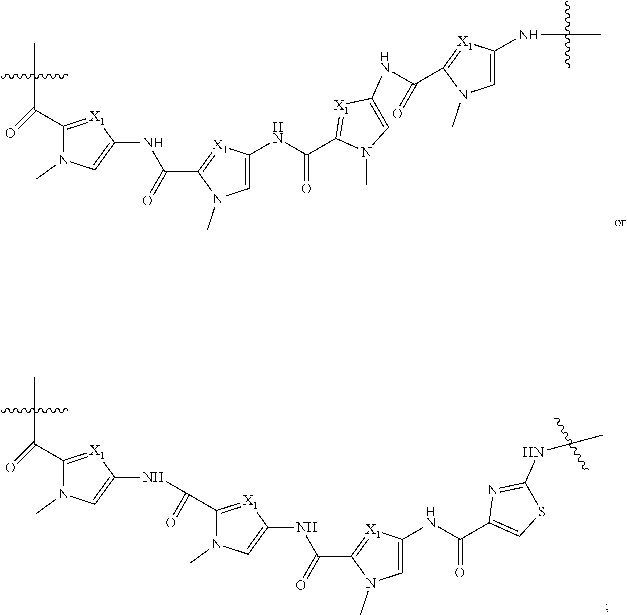
wherein each X₁ independently is CH or N.

83. The conjugate of any one of the preceding claims, wherein E is

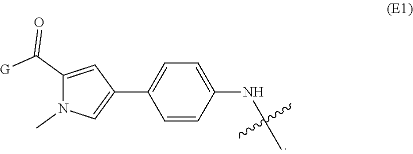

84. The conjugate of any one of the preceding claims, wherein G is 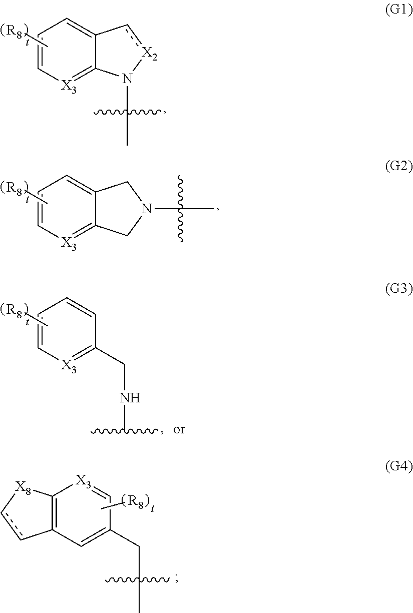
wherein the dotted line in G1 or G4 indicates the presence of a single or double bond.

85. The conjugate of any one of the preceding claims, wherein G is

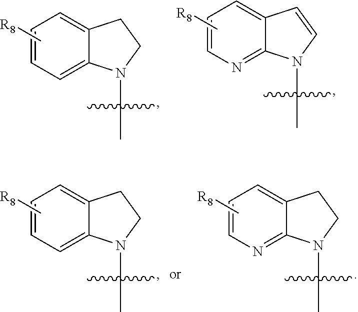

86. The conjugate of any one of the preceding claims, wherein in 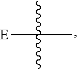
the functional group of E is G or a portion thereof.

87. The conjugate of any one of the preceding claims, wherein in 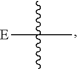
the 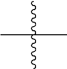
denotes direct or indirect linkage to the PBRM via G or a portion thereof.

88. The conjugate of any one of the preceding claims, wherein in 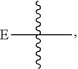
the 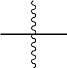
denotes direct or indirect linkage to L^{C} via G or a portion thereof.

89. The conjugate of any one of the preceding claims, wherein in 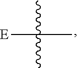
the 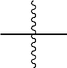
denotes direct or indirect linkage to L^{D} via G or a portion thereof.

90. The conjugate of any one of the preceding claims, wherein in 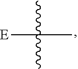
the functional group of E is R₈ or a portion thereof.

91. The conjugate of any one of the preceding claims, wherein in 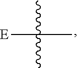
the 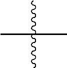
denotes direct or indirect linkage to the PBRM via R₈ or a portion thereof.

92. The conjugate of any one of the preceding claims, wherein in 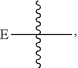
the 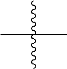
denotes direct or indirect linkage to L^{C} via R₈ or a portion thereof.

93. The conjugate of any one of the preceding claims, wherein in 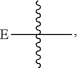
the 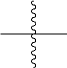
denotes direct or indirect linkage to L^{D} via R₈ or a portion thereof.
The conjugate of any one of the preceding claims, wherein 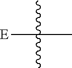
is 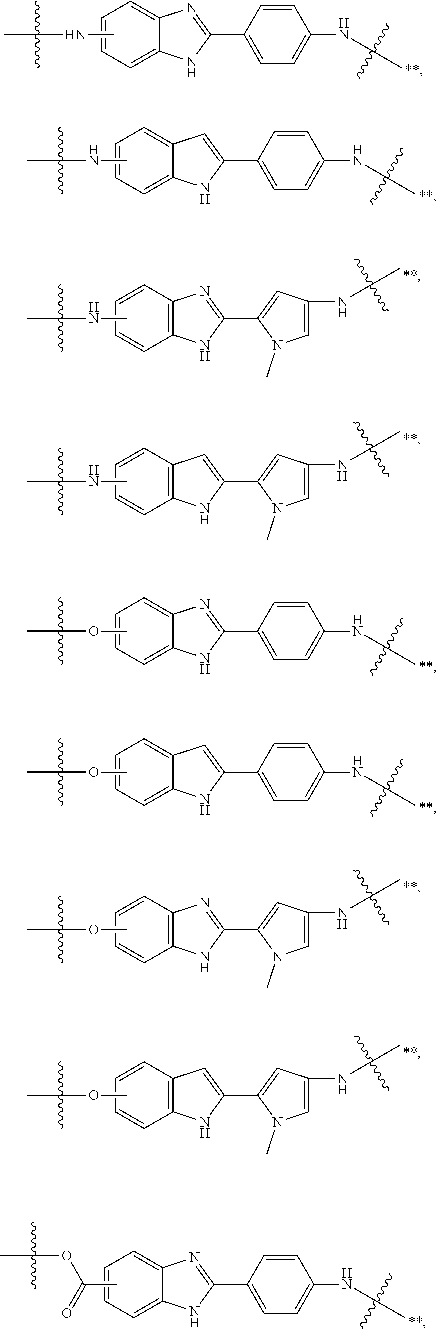 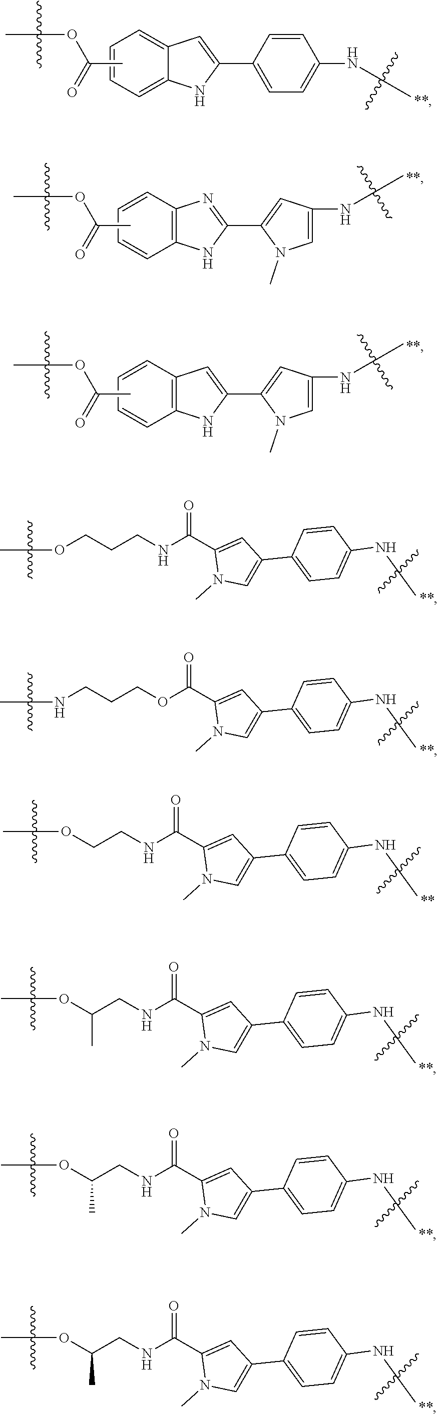 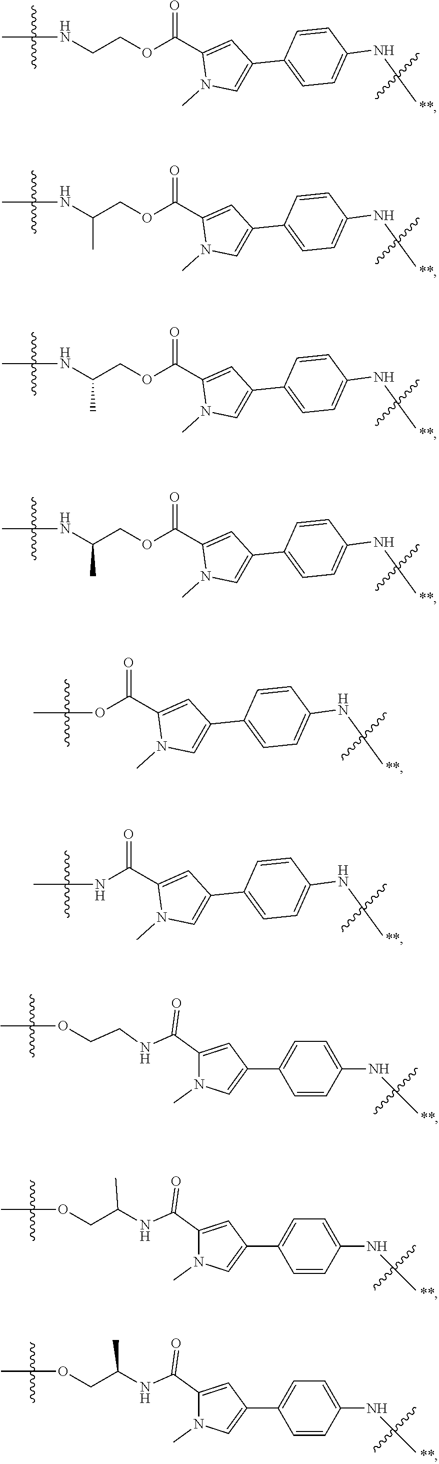 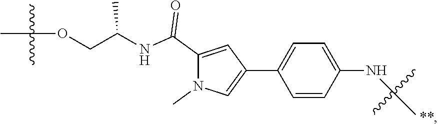 [SMILES: *Nc1ccc(-c2cc(C(=O)N[C@@H](C)CO*)n(C)c2)cc1] 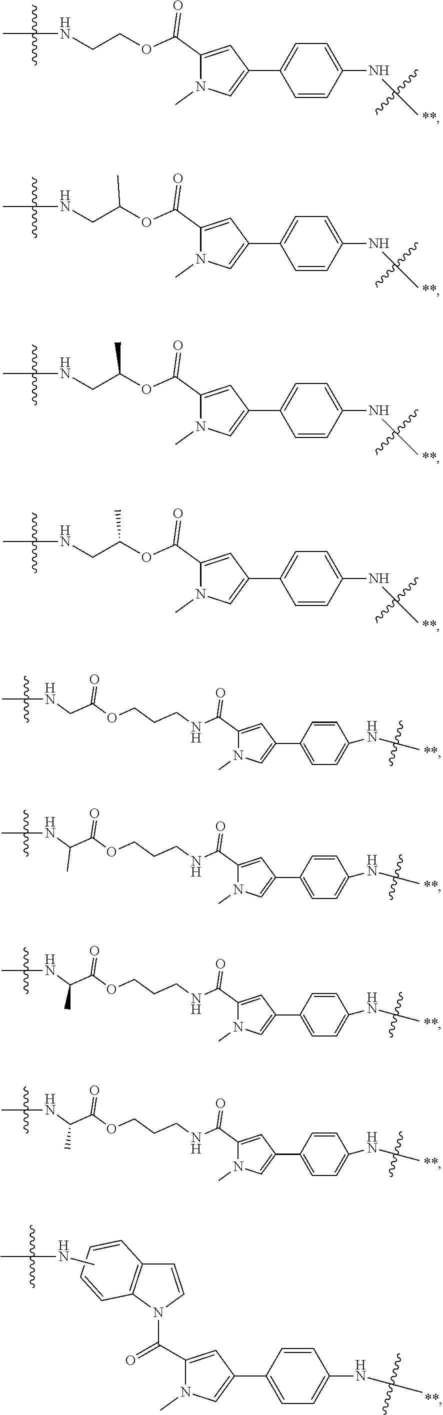 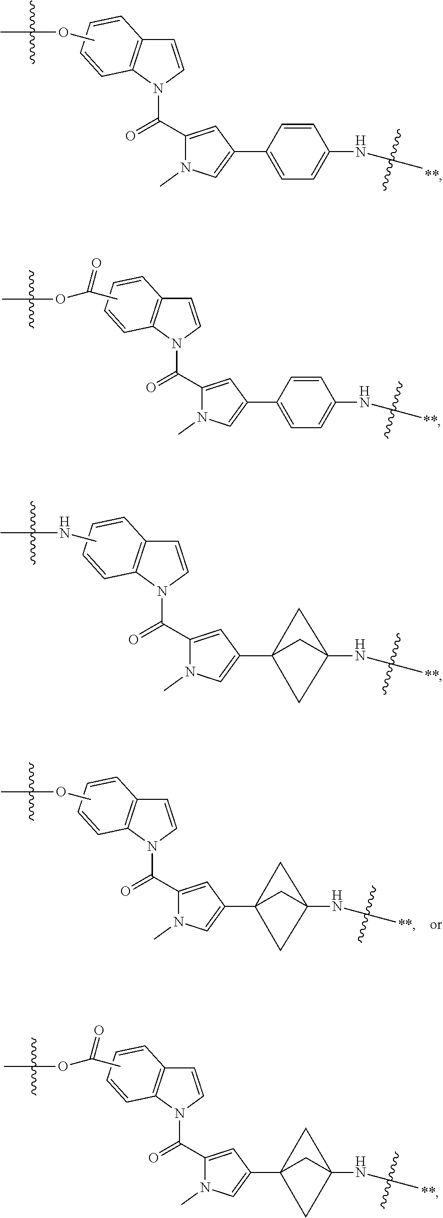
in which 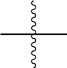
denotes a direct or indirect linkage to the PBRM, L^{C}, or L^{D}, and 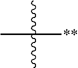
denotes a direct or indirect linkage to a remaining portion of D (e.g., a direct or indirect linkage to A).

94. The conjugate of any one of the preceding claims, wherein 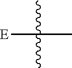
is 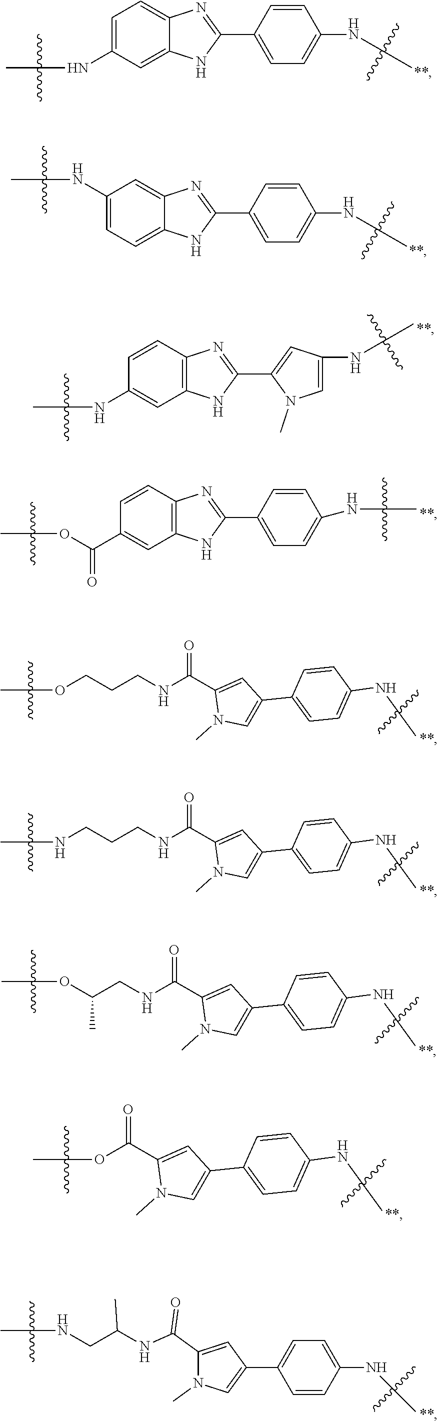 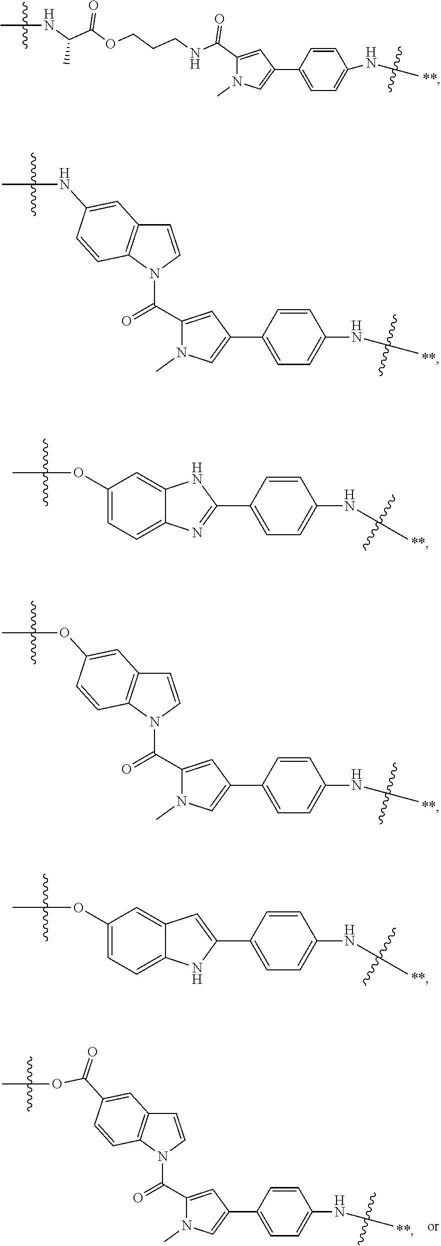 [SMILES: *Nc1ccc(-c2cc(C(=O)NCCCOC(=O)[C@H](C)N*)n(C)c2)cc1 ; *Nc1ccc(-c2cc(C(=O)n3ccc4cc(N*)ccc43)n(C)c2)cc1 ; *Nc1ccc(-c2nc3ccc(O*)cc3[nH]2)cc1 ; *Nc1ccc(-c2cc(C(=O)n3ccc4cc(O*)ccc43)n(C)c2)cc1 ; *Nc1ccc(-c2cc3cc(O*)ccc3[nH]2)cc1 ; *Nc1ccc(-c2cc(C(=O)n3ccc4cc(C(=O)O*)ccc43)n(C)c2)cc1] 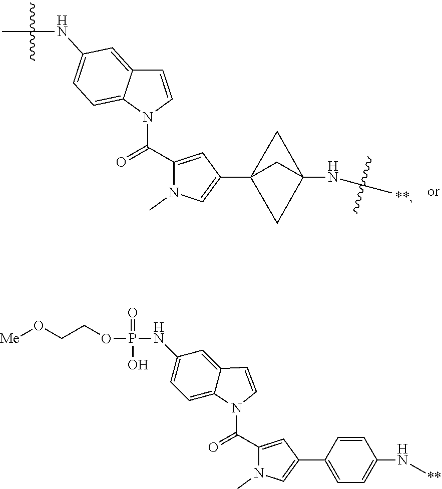 [SMILES: *Nc1ccc2c(ccn2C(=O)c2cc(C34CC(N*)(C3)C4)cn2C)c1 ; CNc1ccc(-c2cc(C(=O)n3ccc4cc(NP(=O)(O)OCCOC)ccc43)n(C)c2)cc1]
in which 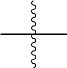
denotes a direct or indirect linkage to the PBRM, L^{C}, or L^{u}, and 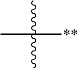
denotes a direct or indirect linkage to a remaining portion of D (e.g., a direct or indirect linkage to A).

95. The conjugate of any one of the preceding claims, wherein 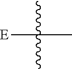
is 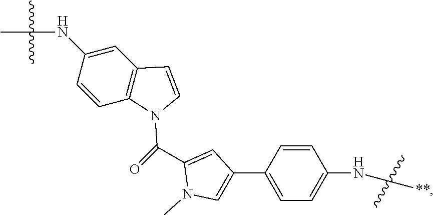
in which 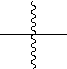
denotes a direct or indirect linkage to the PBRM, L^{C}, or L^{D}, and 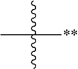
denotes a direct or indirect linkage to a remaining portion of D (e.g., a direct or indirect linkage to A).

96. The conjugate of any one of the preceding claims, wherein E is 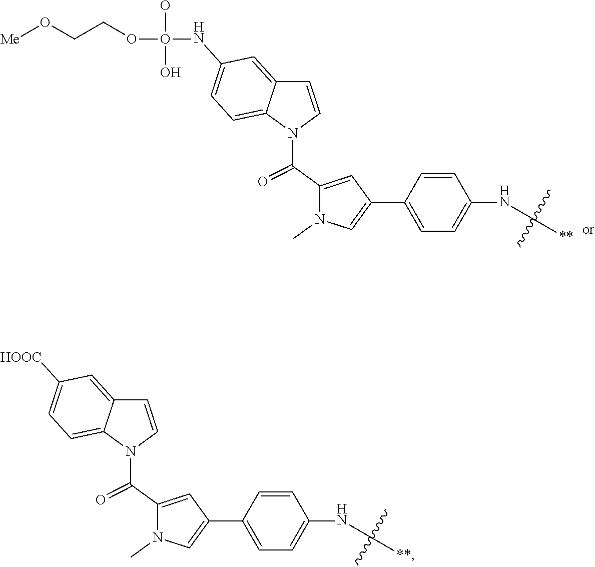
in which 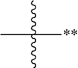
denotes a direct or indirect linkage to a remaining portion of D (e.g., a direct or indirect linkage to A).

97. The conjugate of any one of the preceding claims, wherein E is 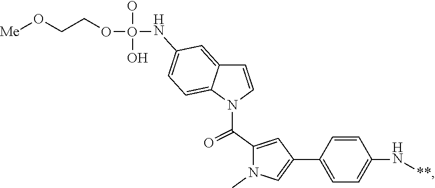
in which 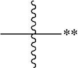
denotes a direct or indirect linkage to a remaining portion of D (e.g., a direct or indirect linkage to A).

98. The conjugate of any one of the preceding claims, wherein E is 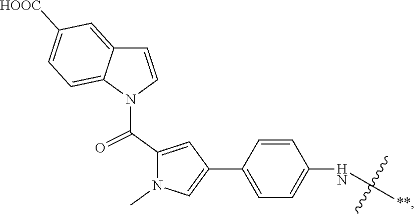
in which 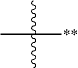
denotes a direct or indirect linkage to a remaining portion of D (e.g., a direct or indirect linkage to A).

99. The conjugate of any one of the preceding claims, wherein the PBD drug moiety (D) is of any one of Formulae (IX-a) to (IX-r): 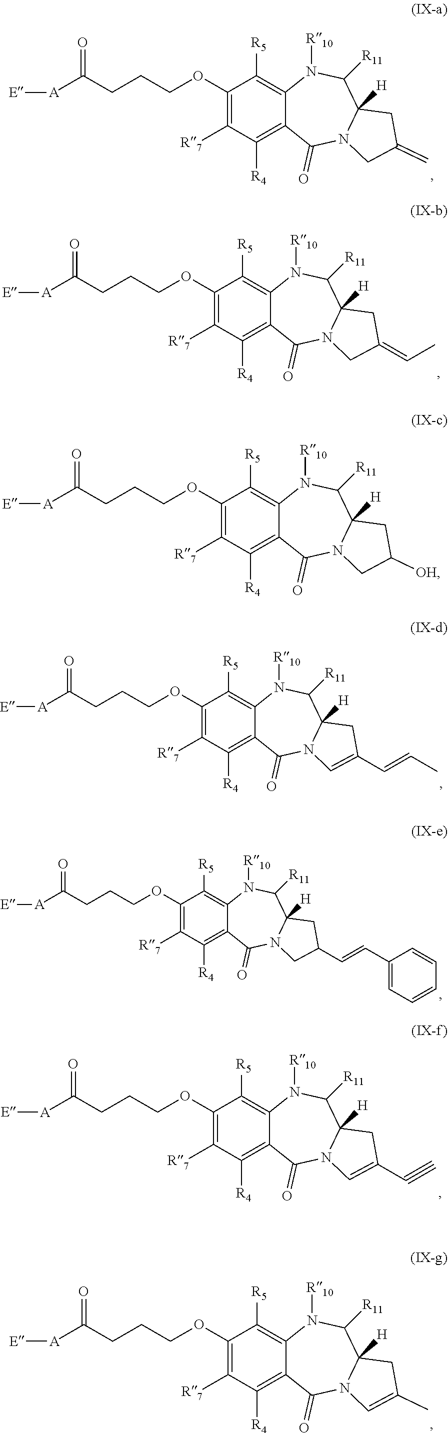 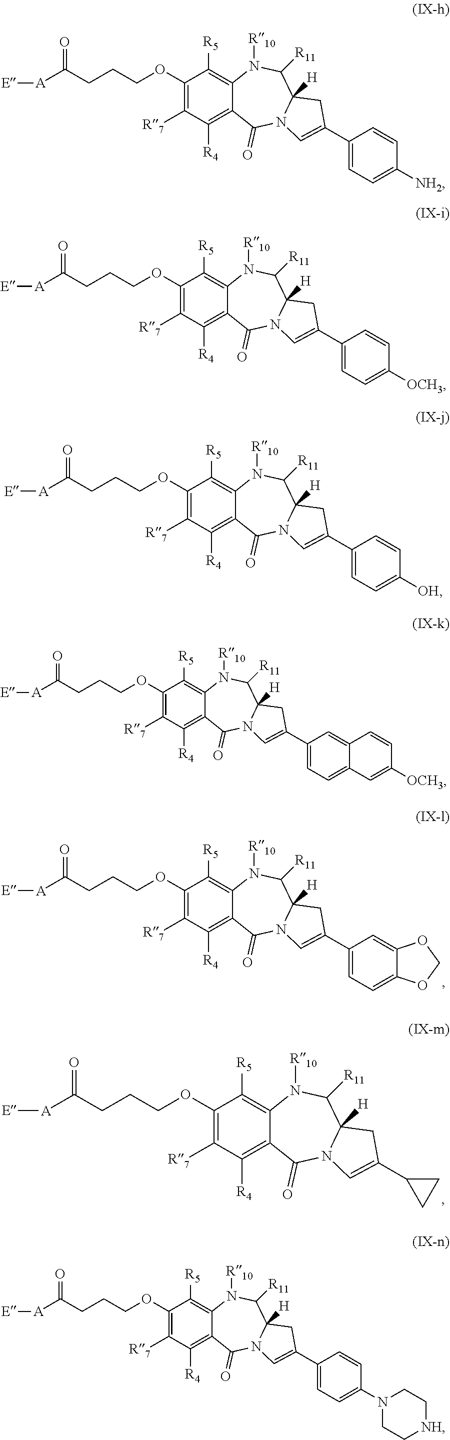 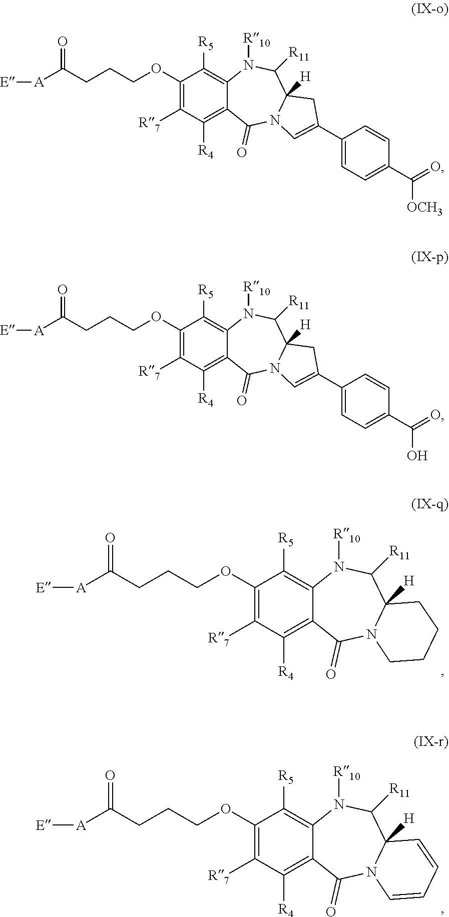
a tautomer thereof, a pharmaceutically acceptable salt or solvate thereof, or a pharmaceutically acceptable salt or solvate of the tautomer.

100. The conjugate of any one of the preceding claims, wherein the PBD drug moiety (D), prior to being connected to another portion of the conjugate, corresponds to a compound selected from the compounds listed in Table 1, tautomers thereof, pharmaceutically acceptable salts or solvates thereof, or pharmaceutically acceptable salts or solvates of the tautomers.

101. The conjugate of any one of the preceding claims, wherein the PBD drug moiety (D), prior to being connected to another portion of the conjugate, corresponds to a compound of any one of Formula (XIIIa) to (XIIIm): 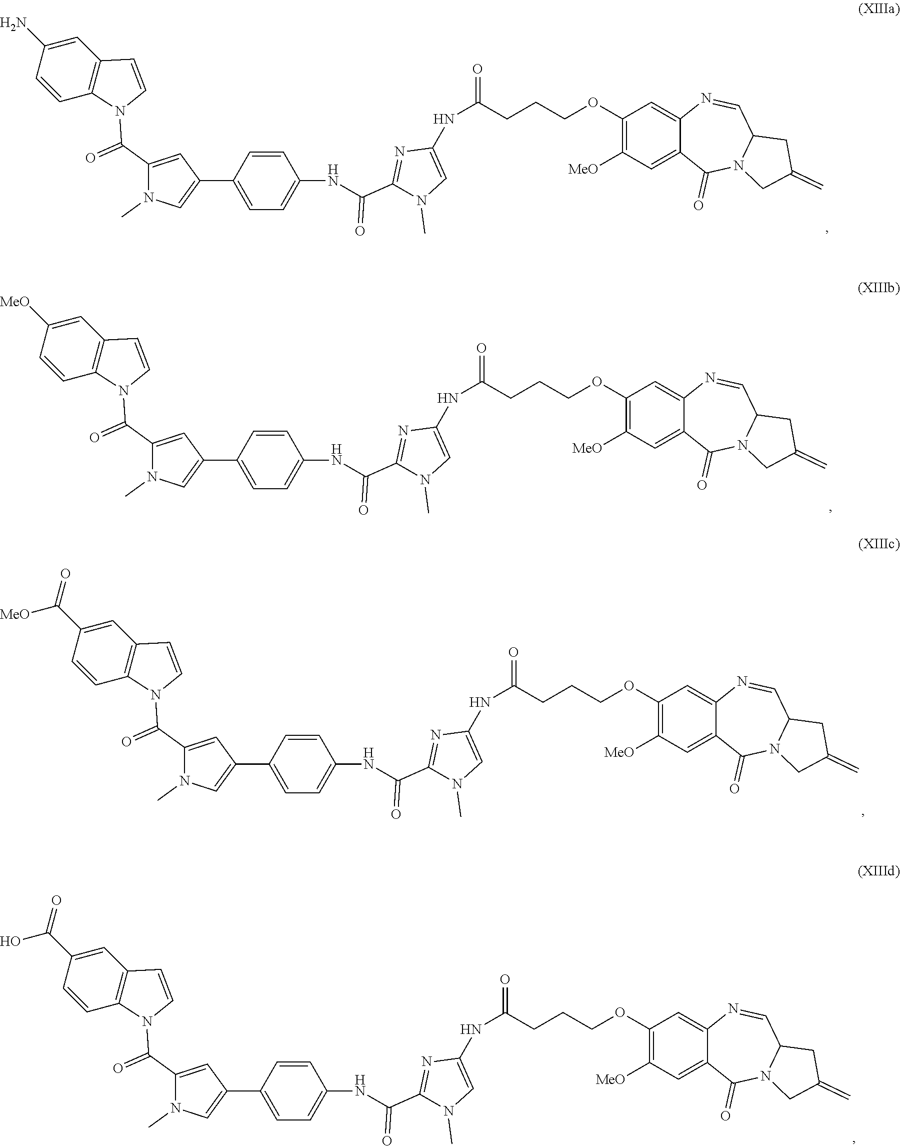 [SMILES: C=C1CC2C=Nc3cc(OCCCC(=O)Nc4cn(C)c(C(=O)Nc5ccc(-c6cc(C(=O)n7ccc8cc(N)ccc87)n(C)c6)cc5)n4)c(OC)cc3C(=O)N2C1 ; C=C1CC2C=Nc3cc(OCCCC(=O)Nc4cn(C)c(C(=O)Nc5ccc(-c6cc(C(=O)n7ccc8cc(OC)ccc87)n(C)c6)cc5)n4)c(OC)cc3C(=O)N2C1 ; C=C1CC2C=Nc3cc(OCCCC(=O)Nc4cn(C)c(C(=O)Nc5ccc(-c6cc(C(=O)n7ccc8cc(C(=O)OC)ccc87)n(C)c6)cc5)n4)c(OC)cc3C(=O)N2C1 ; C=C1CC2C=Nc3cc(OCCCC(=O)Nc4cn(C)c(C(=O)Nc5ccc(-c6cc(C(=O)n7ccc8cc(C(=O)O)ccc87)n(C)c6)cc5)n4)c(OC)cc3C(=O)N2C1] 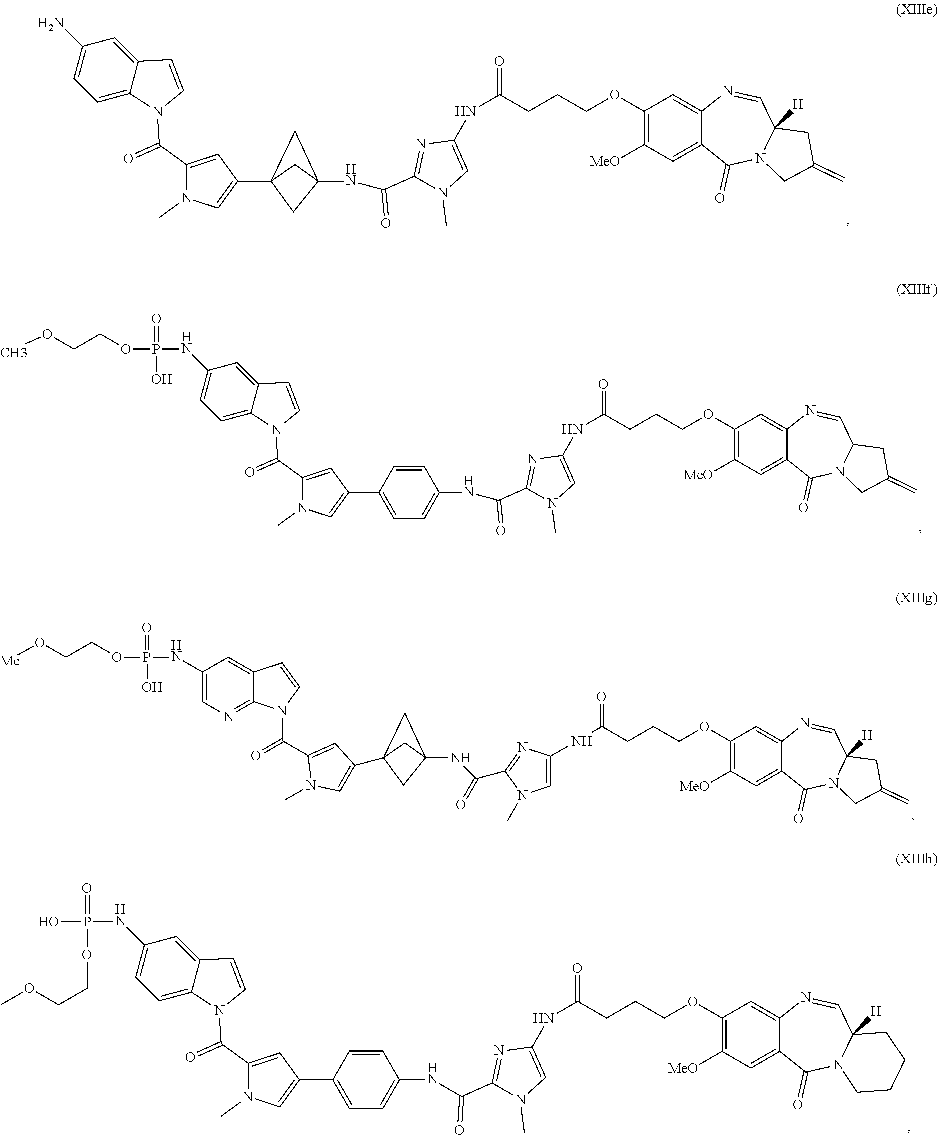 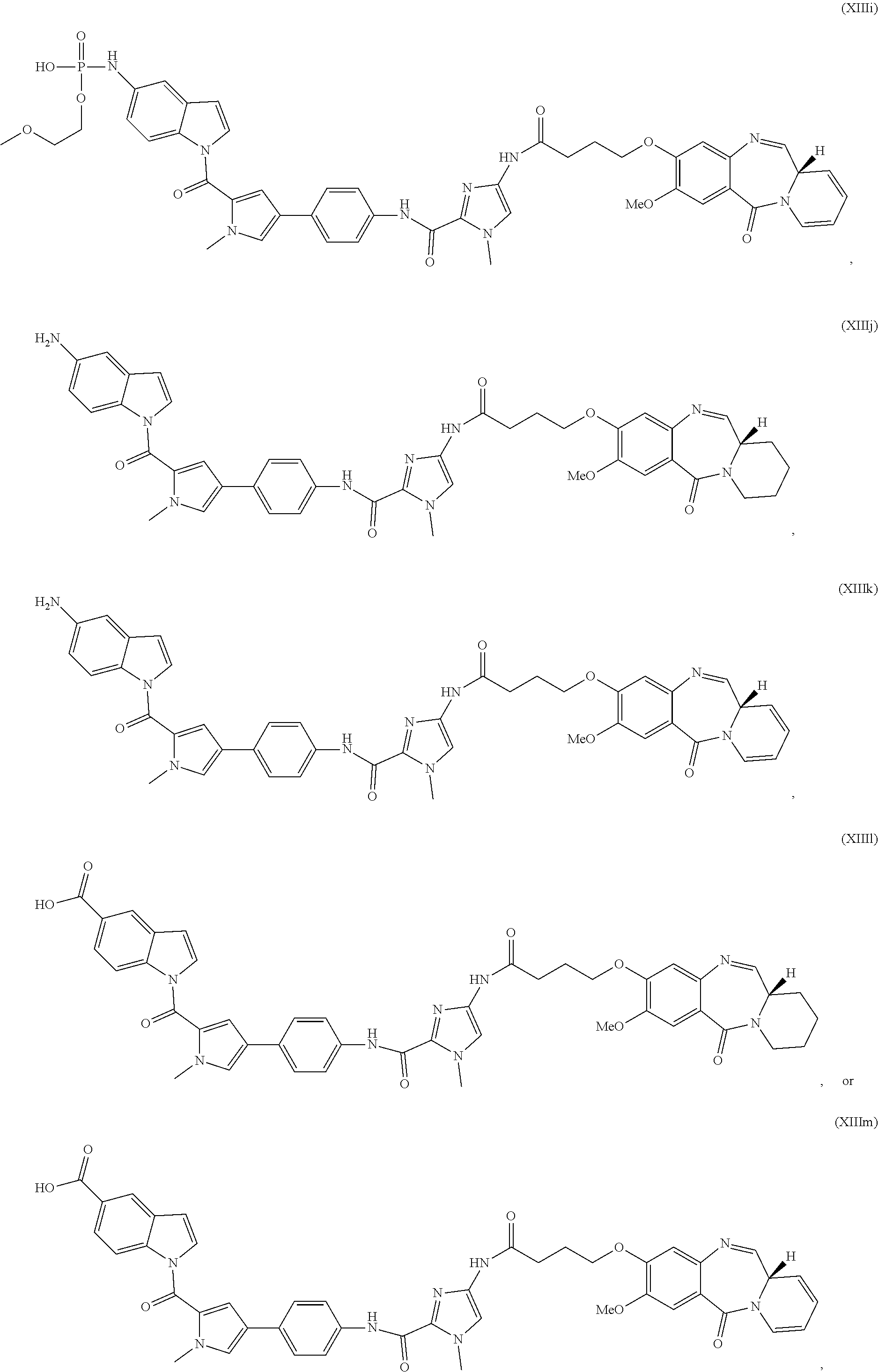
a tautomer thereof, a pharmaceutically acceptable salt or solvate thereof, or a pharmaceutically acceptable salt or solvate of the tautomer.

102. The conjugate of any one of the preceding claims, wherein the PBD drug moiety (D) is selected from the conjugates listed in Table 1A, tautomers thereof, pharmaceutically acceptable salts or solvates thereof, and pharmaceutically acceptable salts or solvates of the tautomers.

103. The conjugate of any one of the preceding claims, being selected from the conjugates Formula (XIVa) to (XIVx): 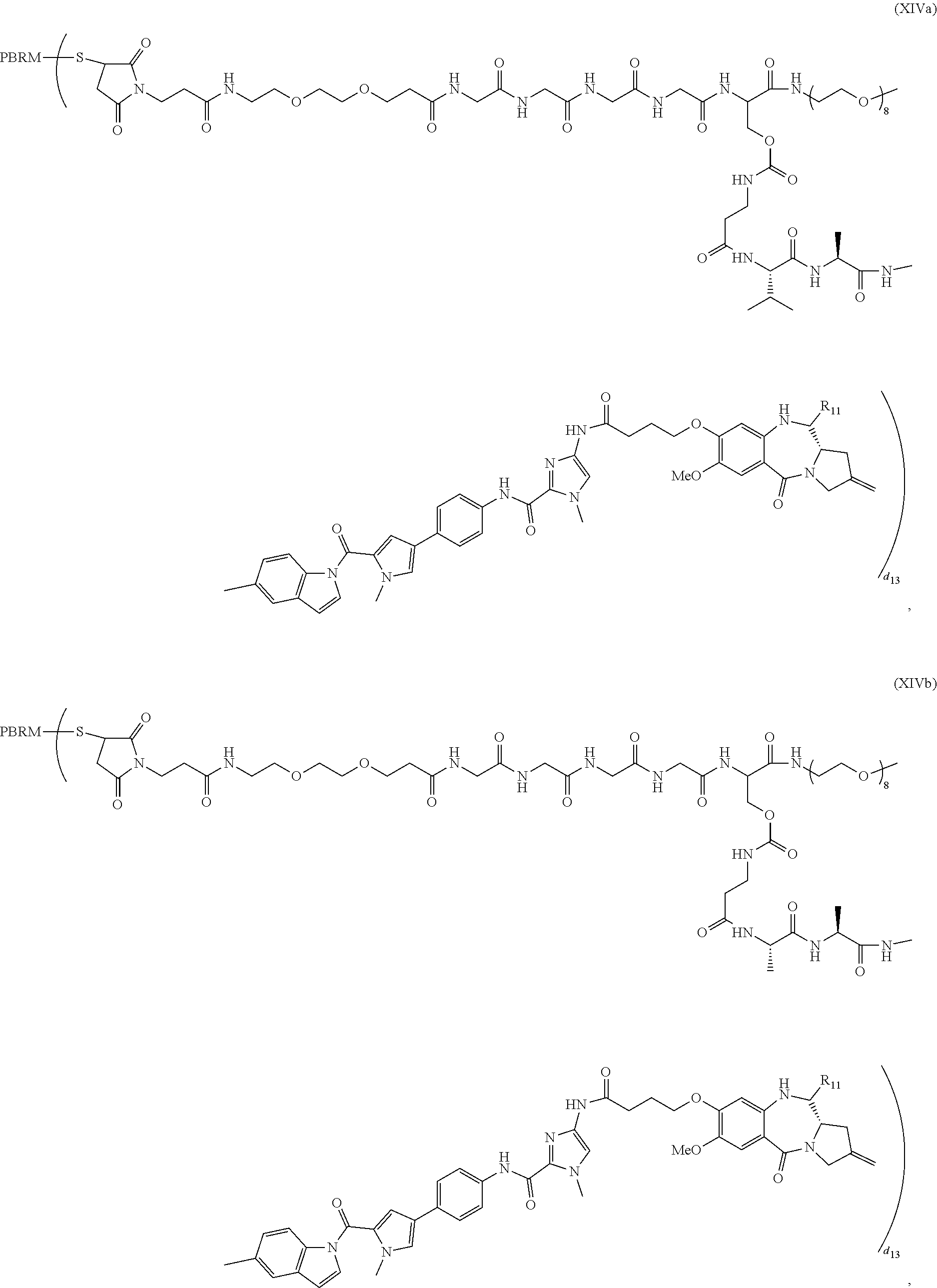 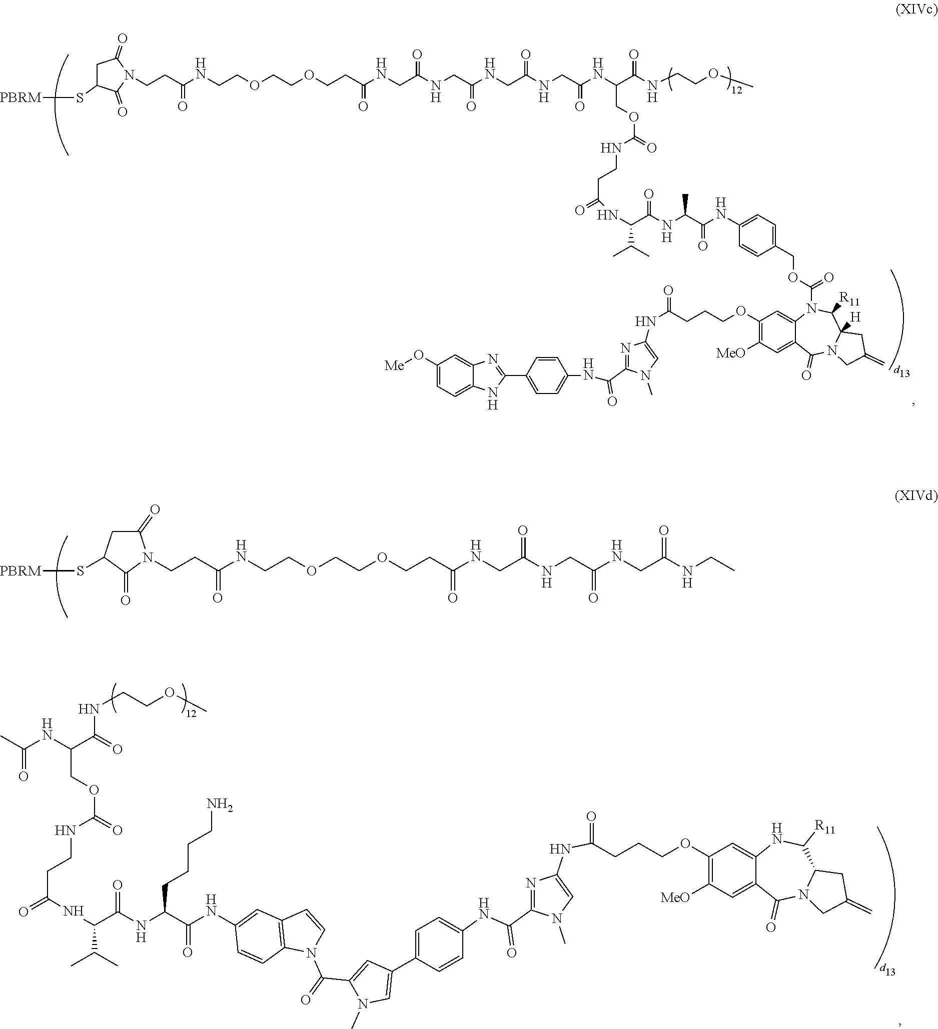 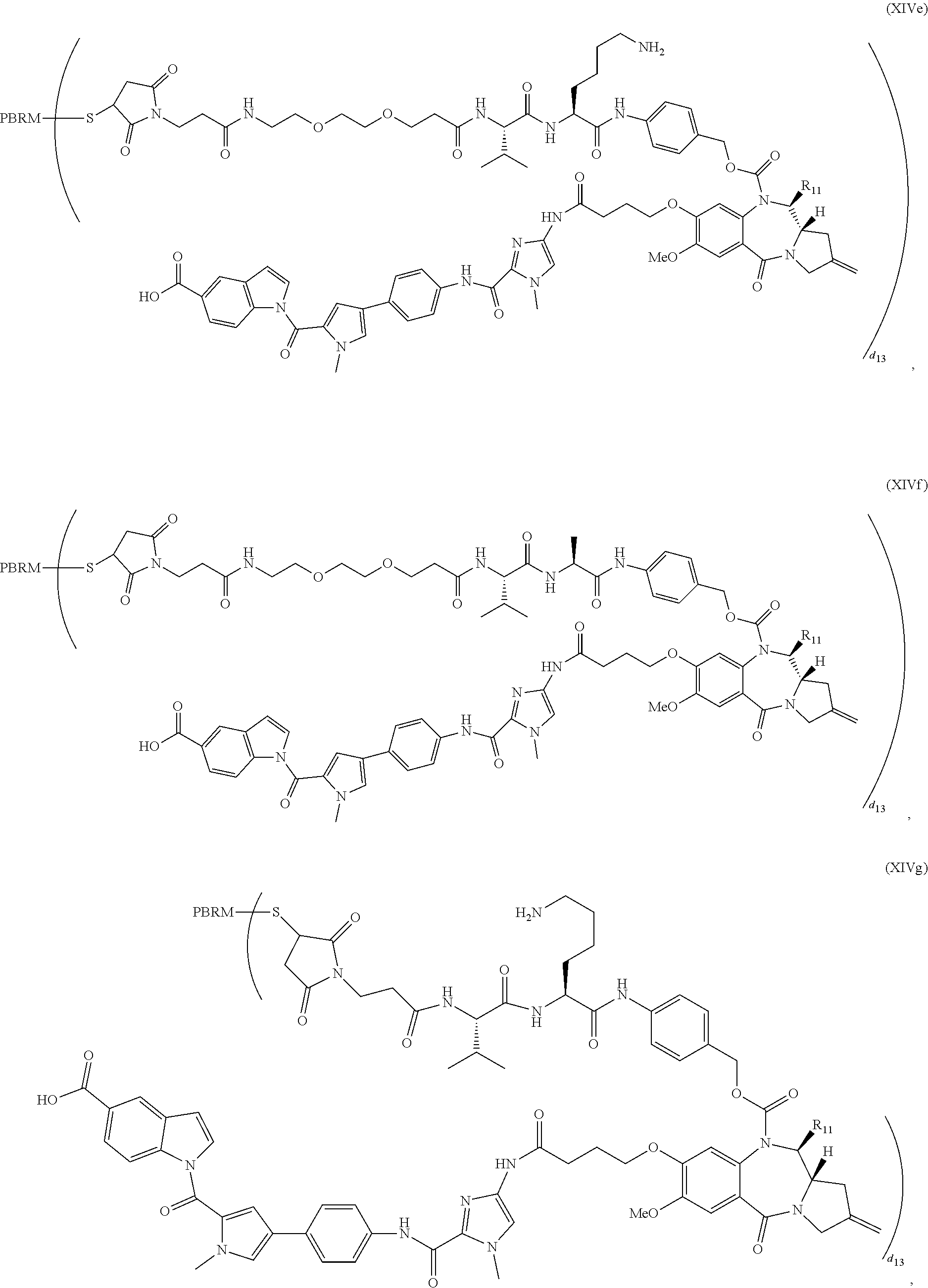 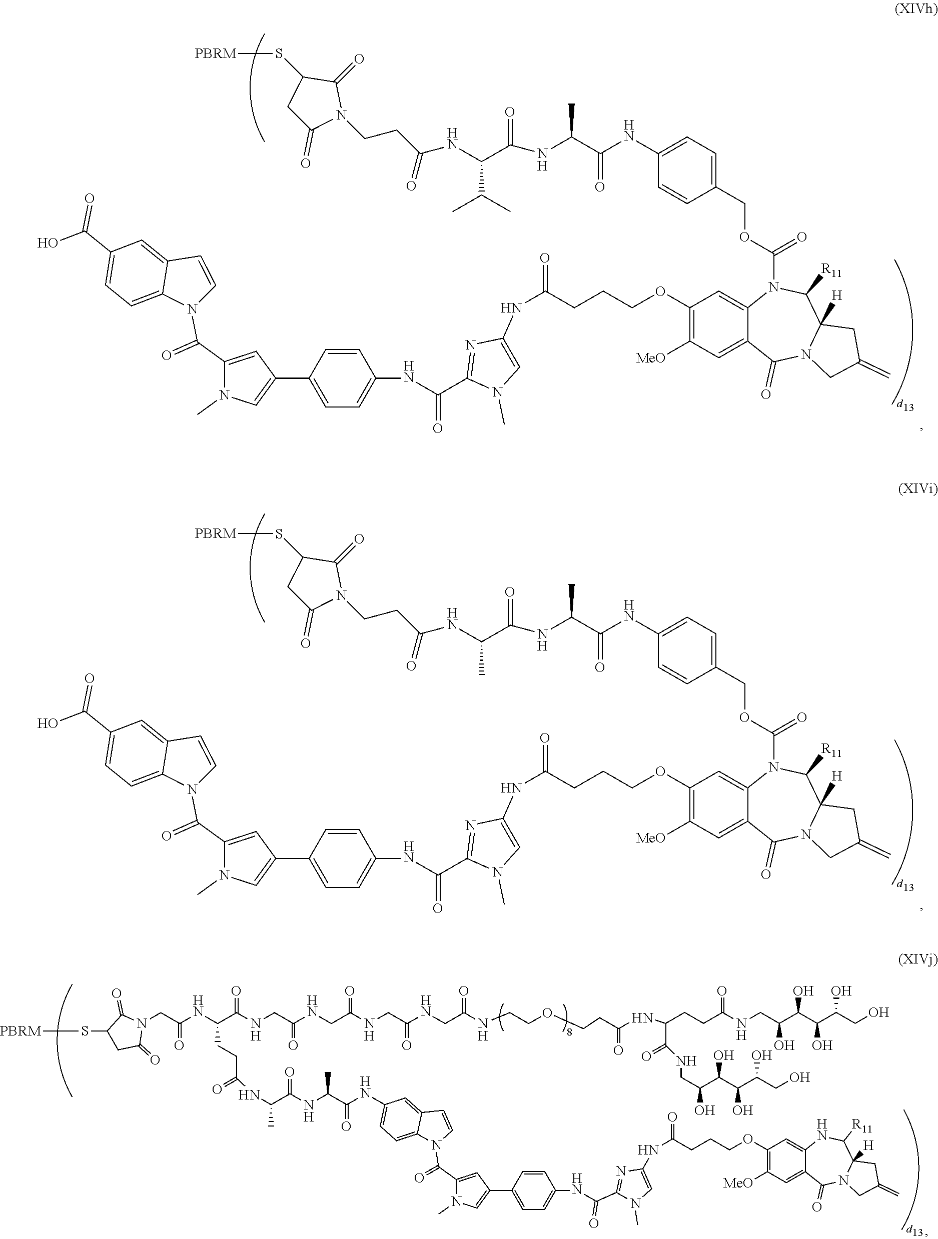 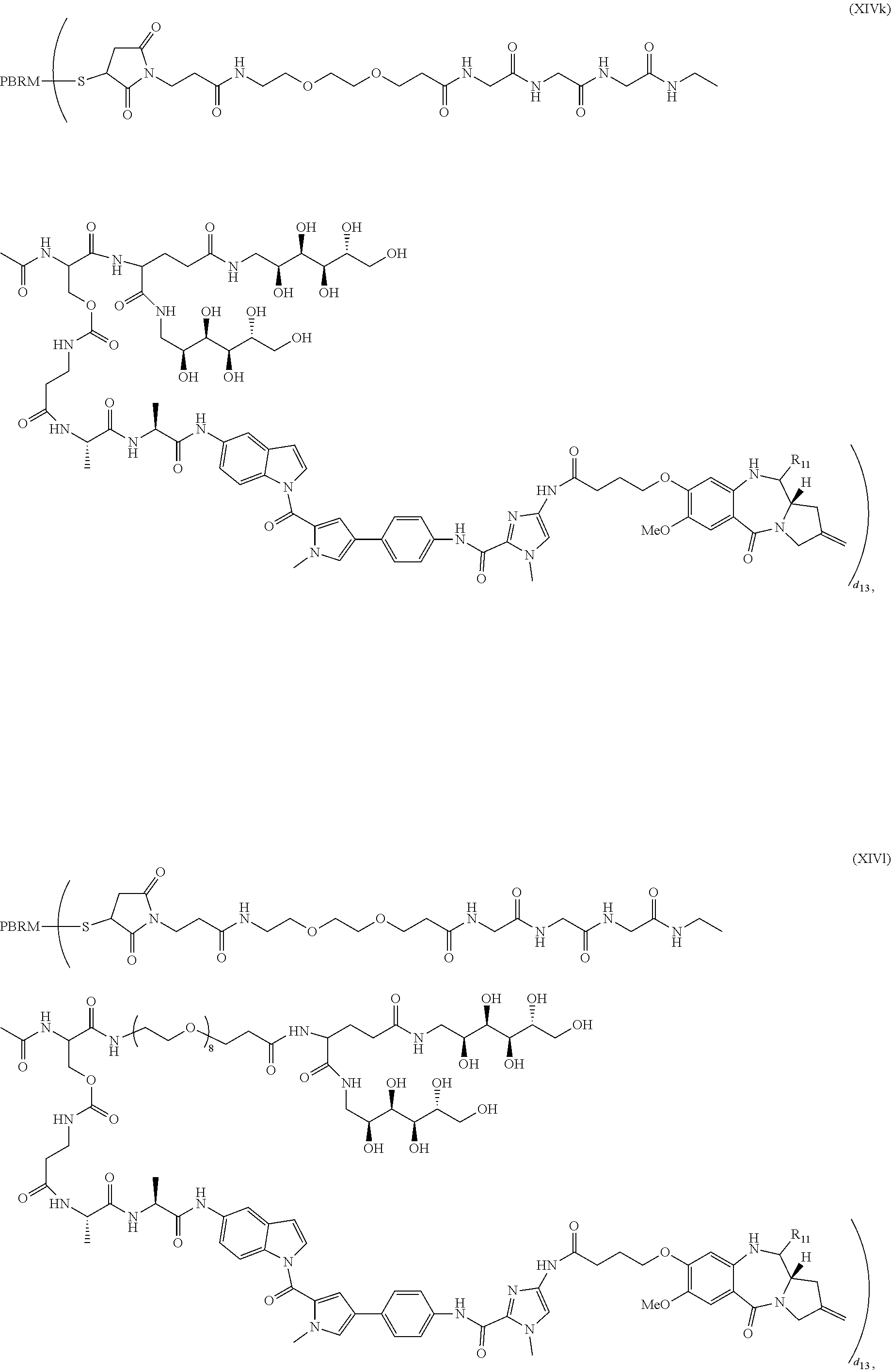 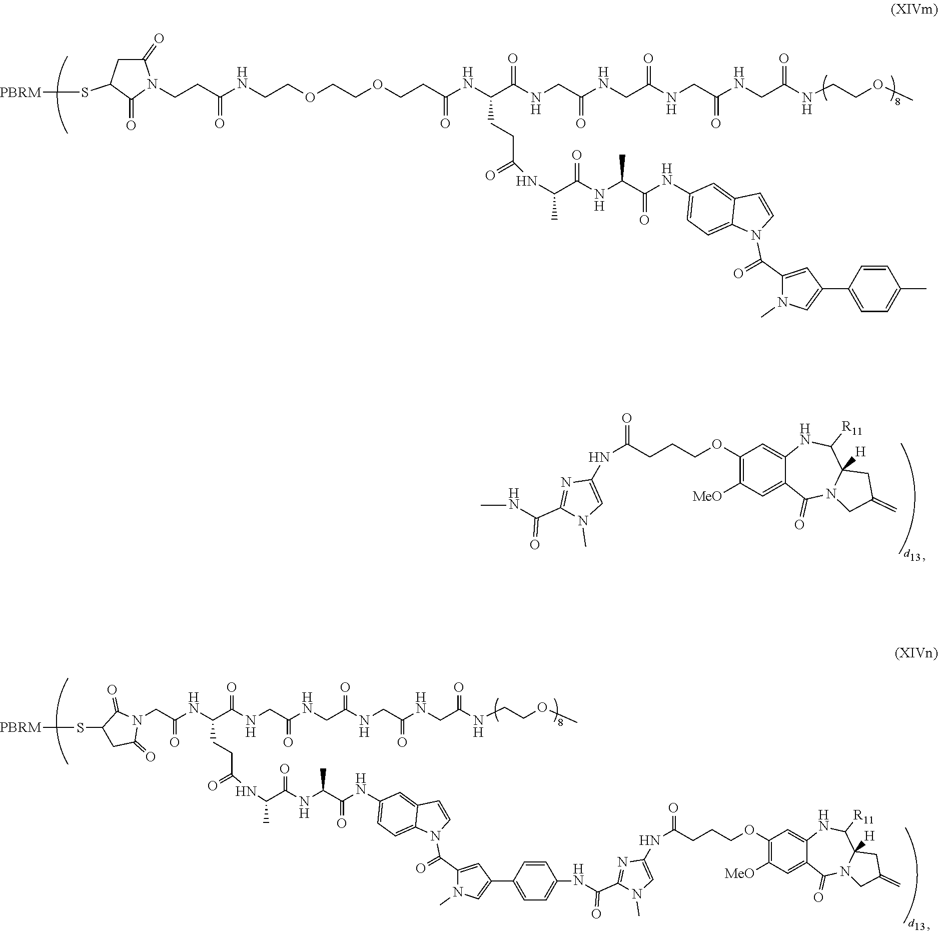 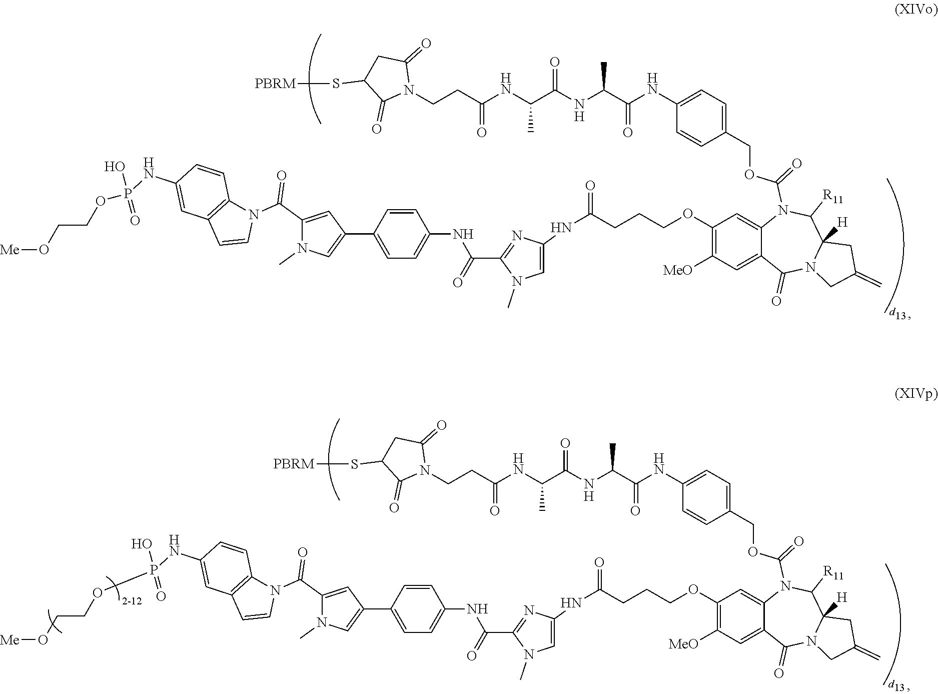 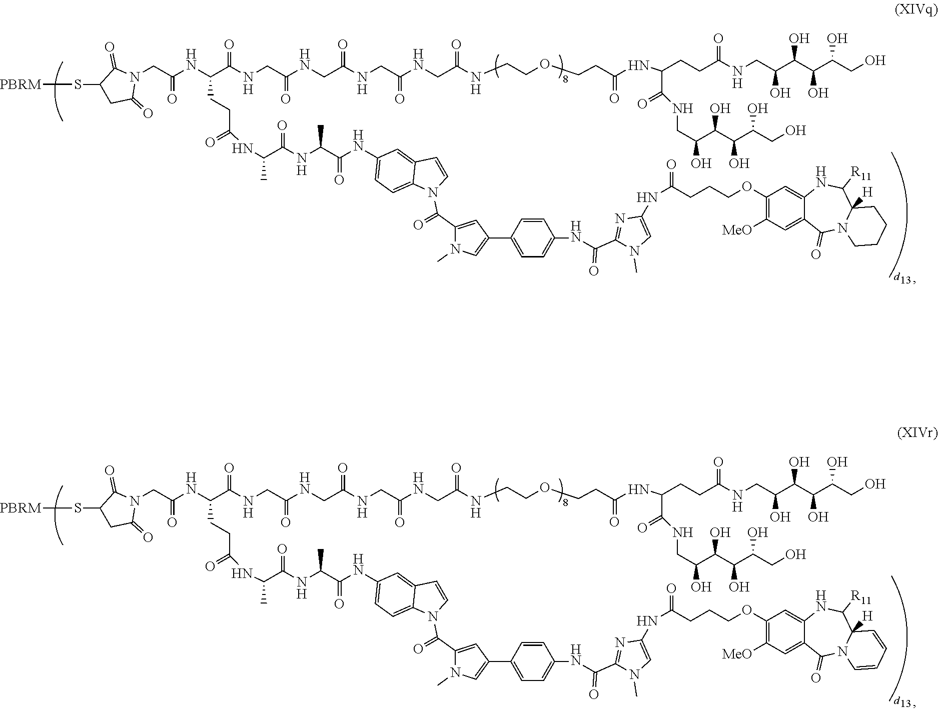 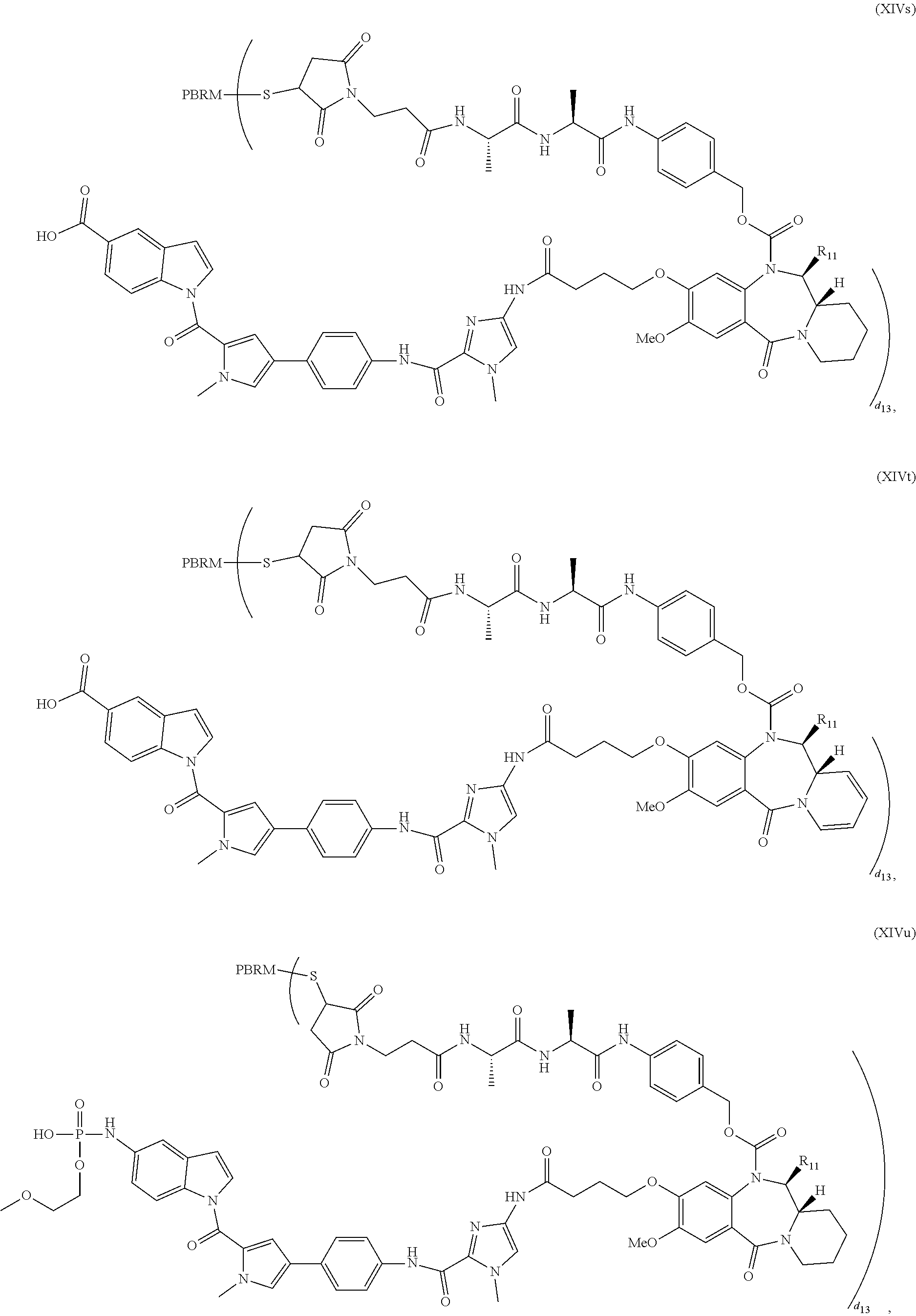 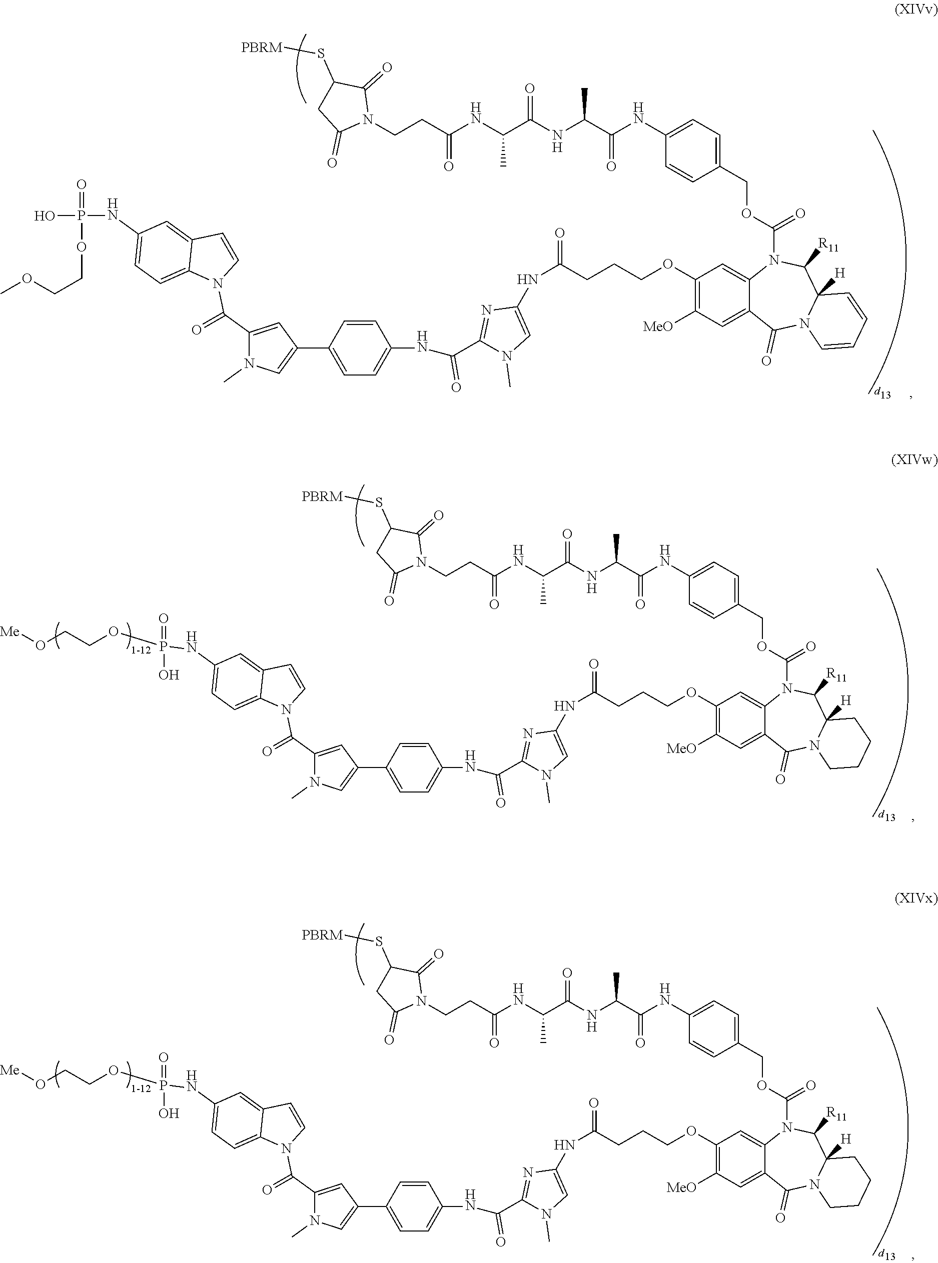
tautomers thereof, pharmaceutically acceptable salts or solvates thereof, and pharmaceutically acceptable salts or solvates of the tautomers, and
wherein d₁₃ is 3 to 5.

104. The conjugate of any one of the preceding claims, being selected from the conjugates Formula (XIVi), (XIVj) and (XIVo): 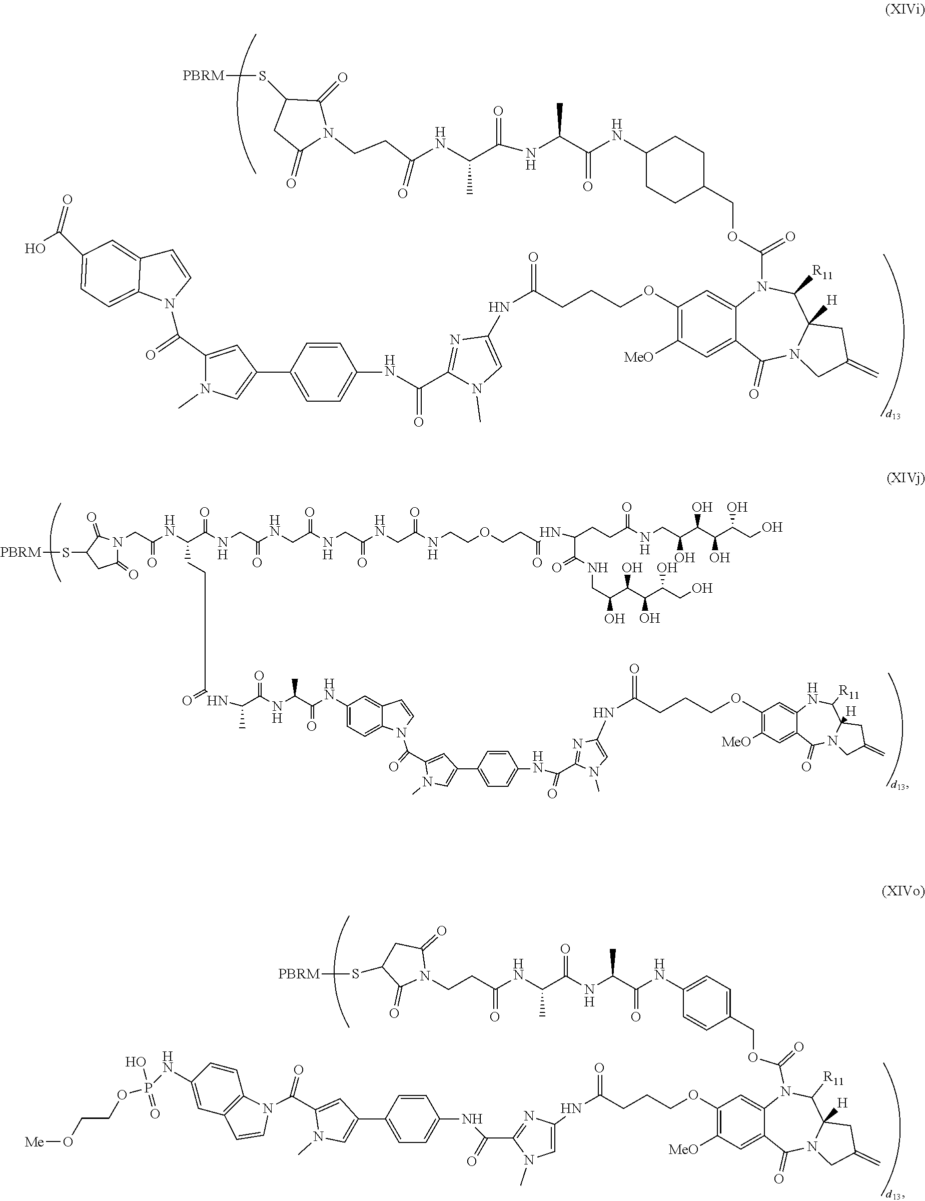
tautomers thereof, pharmaceutically acceptable salts or solvat of, and pharmaceutically acceptable salts or solvates of the tautomers.

105. The conjugate of Formula (XIVo): 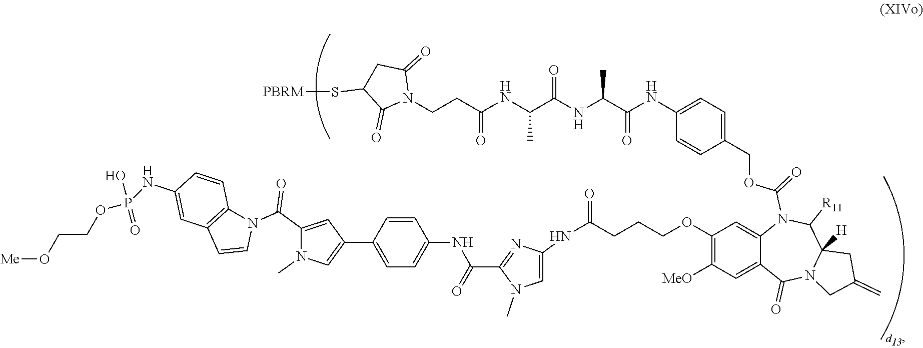
tautomers thereof, pharmaceutically acceptable salts or solvates thereof, and pharmaceutically acceptable salts or solvates of the tautomers.

106. The conjugate of any one of the preceding claims, being selected from the conjugates listed in Table 2, tautomers thereof, pharmaceutically acceptable salts or solvates thereof, and pharmaceutically acceptable salts or solvates of the tautomers.

107. A pharmaceutical composition comprising the conjugate of any one of the preceding claims and a pharmaceutically acceptable carrier.

108. A method of treating or preventing a disease or disorder, comprising administering to a subject in need thereof a pharmaceutically effective amount of the conjugate of any one of the preceding claims.

109. The method of any one of the preceding claims, wherein the disease or disorder is cancer.

110. The conjugate of any one of the preceding claims for use in treating or preventing a disease or disorder.

111. Use of the conjugate of any one of the preceding claims in treating or preventing a disease or disorder.

112. Use of the conjugate of any one of the preceding claims in the manufacture of a medicament for treating or preventing a disease or disorder.
